(19) United States
(12) Patent Application Publication
(10) Pub. No.: US 2003/0068616 A1
(43) Pub. Date: Apr. 10, 2003

(54) Drug discovery assays based on microcompetition for a limiting GABP complex

(76) Inventor: Hanan Polansky, Rochester, NY (US)

Correspondence Address: Hanan Polansky, 3159 S. Winton Rd., Rochester, NY, 14623, US

(21) Appl. No.: 10/223,050

(22) Filed: Aug. 14, 2002

Related U.S. Application Data
(63) Continuation-in-part of application No. 09/732,360, filed on Dec. 7, 2000.

(51) Int. Cl. C12Q 1/70; G01N 33/567; C12N 15/86; C12N 5/08

(52) U.S. Cl. 435/5; 435/7.21; 435/456; 435/320.1; 435/325; 435/366

(57) ABSTRACT

A recent discovery showed that microcompetition for GABP between a foreign polynucleotide and a cellular GABP regulated gene is a risk factor for some of the major chronic diseases, such as obesity, cancer, atherosclerosis, stroke, osteoarthritis, diabetes, asthma, and other autoimmune diseases. The invention uses this novel discovery to present assays for screening compounds based on their effectiveness in modulating such microcompetition, or the effects of such microcompetition on the cell. The selected compounds can be used in treatment of these chronic diseases. The invention also presents assays for screening compounds that can be used in treatment of chronic diseases resulting from other foreign polynucleotide-type disruptions.

### BACKGROUND OF THE INVENTION

The cause of many cases of the major chronic diseases is unknown. Therefore, treatment is focused on clinical symptoms associated with the disease rather than the cause. As a result, in many cases, the treatment shows limited efficacy and serious negative side effects.

Recently, the Natlonal Cancer Institute (NIH Guide 2000¹) announced a program aimed to “reorganize the “front-end,” or gateway, to drug discovery in cancer. The new approach promotes a three stage discovery process; first, discovery of the molecular mechanisms underlying neoplastic transformations, cancer growth and metastasis; second, selection of a novel molecular target within the discovered biochemical pathway associated with the disease state; finally, design of a new drug that modifies the selected target. The program encourages moving away from screening based on a clinical effects, such as tumor cell shrinkage, either in vivo or in vitro, to screening, or drug design, based on molecular effects. According to the NCI, screening by a desired clinical effect identified drugs that traditionally demonstrated clear limitations in patients, while screening by a desired molecular effect should produce more efficacious and specific drugs.

The best drugs reverse the molecular events that cause a disease. Following the discovery of microcompetition between foreign polynucleotides and cellular genes as the cause of many chronic disease cases, the present invention presents methods for treating chronic diseases, methods for evaluating the effectiveness of a compound for use in modulating the progression of chronic diseases, and methods for determining whether a subject has a chronic disease, or has an increased risk of developing clinical symptoms associated with such disease.

### BRIEF SUMMARY OF THE INVENTION

In one aspect, the invention presents methods for treating chronic diseases. In a preferred embodiment, the methods feature administration to a subject a therapeutically effective amount of a pharmaceutical or nutraceutical composition that attenuates microcompetition between a foreign polynucleotide and a cellular polynucleotide, attenuates an effect of such microcompetition, or attenuates an effect of another foreign polynucleotide-type disruption. A pharmaceutical or nutraceutical composition may include, but not limited to, small molecule (organic or inorganic), polynucleotide, polypeptide or antibody.

For example, to ameliorate a disease symptom resulting from microcompetition between a foreign polynucleotide and a cellular polynucleotide, a pharmaceutical composition can be administered to the subject that reduces the cellular copy number of the foreign polynucleotide, reduces complex formation between the foreign polynucleotide and a cellular transcription factor, increases complex formation between the microcompeted cellular transcription factor and the cellular polynucleotide, or reverses an effect of microcompetition on the expression or activity of a polypeptide with expression regulated by the cellular polynucleotide. For example, in the case of a p300/cbp virus and the cellular Rb gene, a pharmaceutical composition can be administered to the subject that reduces the copy number of the p300/cbp virus by, for instance, reducing viral replication, reduces binding of a p300/cbp transcription factor, such as GABP, to the p300/cbp virus, increases expression of the p300/cbp transcription factor, increases binding of the p300/cbp transcription factor to the Rb promoter by, for instance, stimulating phosphorylation of the p300/cbp transcription factor, or increases expression of Rb, through, for instance, transfection of an exogenous Rb gene, reduced degradation of the Rb protein, or administration of exogenous Rb protein (see more examples below).

In the case of another foreign polynucleotide-type disruption, for example, the composition may reverse the effects of such disruption. For instance, microcompetition with a p300/cbp virus reduces expression of Rb. A mutation can also reduce the expression of Rb. Therefore, such mutation is a foreign polynucleotide-type disruption. Microcompetition with a p300/cbp virus can result in cancer, and, therefore, a mutation in the Rb promoter that reduces Rb expression can also result in cancer. To ameliorate the symptoms of cancer resulting from such mutation in the Rb gene, a pharmaceutical composition can be administered to the subject that stimulates complex formation between a p300/cbp transcription factor and Rb.

In second aspect, the invention provides assays for screening test compounds to find compounds that modulate microcompetition between a foreign polynucleotide and a cellular polynucleotide, an effect of such microcompetition, or an effect of another foreign polynucleotide-type disruption.

A further aspect of the invention provides methods for determining the risk of developing the molecular, cellular and clinical symptoms associated with a chronic disease. The method may include detecting in a biological sample obtained from a subject at least one of the following: (i) a foreign polynucleotide, specifically, a p300/cbp virus (ii) modified expression or bioactivity of a gene susceptible to microcompetition with a foreign polynucleotide, specifically, a p300/cbp regulated gene (iii) presence of a genetic lesion in a gene susceptible to microcompetition with a foreign polynucleotide, specifically, a gene encoding a p300/cbp factor, a p300/cbp regulated gene, p300/cbp factor kinase or p300/cbp phosphatase, or p300/cbp agent (iv) presence of a genetic lesion in a DNA binding box of a p300/cbp transcription factor.

### BRIEF DESCRIPTION OF THE FIGURES

FIG. 1 shows the relation between molar ratio of pSV2Neo/hMT-II_{A}-CAT and relative CAT activity.

FIG. 2 shows the relation between molar ratio of bgal/CAT and relative CAT activity.

FIG. 3 shows the amount of COL1A2 RNA measured in cells grown at temperature permissive (T) or non-permissive (N) for transformation.

FIG. 4 shows the effect of infection with HIV-1, heat inactivated HIV-1 and mock-infection on CD18 expression over time.

FIG. 5 shows a schematic illustration of the extracellular signaling cascade and its effect on GABP.

FIG. 6 shows a schematic illustration of the activation of MAPK by MEK-1, and deactivation of MAPK by PP2A, PTP1B, or MKP-1.

FIG. 7 shows a schematic illustration of the relationship between ERK signaling and microcompetition for available GABP.

FIG. 8 shows a schematic illustration of how phosphorylated GABP stimulates the transcription of the sensitized receptor and how the new receptors increase the sensitivity of the pathway to changes in concentration of GABP kinase agent.

FIG. 9 shows a schematic illustration of feedback inhibition involving GABP.

FIG. 10 shows a schematic illustration of the effect of a downstream control relative to a sensitized receptor.

FIG. 11 shows the effect of HSV-1 and LPS exposure on TF procoagulant activity (PCA) of human umbilical vein endothelial cells.

FIG. 12 shows a schematic illustration of the effects of LPS, RSVL and RA on NF-κB and ETS sites of the TF gene.

FIG. 13 shows a schematic illustration of the P450 mediated oxidation of arachidonic acid.

FIG. 14 shows the relation between MAPK activity and arachidonic acid metabolites.

FIG. 15 shows the number of viable cells following transfection with pBARB and the “empty vector” pSV-neo.

FIG. 16 shows accumulation of triglyceride, assayed by oil red staining in untreated F442A cells, or following transfection with the WT, and the “empty vector” pZIPNeo.

FIG. 17 shows the percent reverse transmigration of peripheral blood mononuclear cells as a function of time.

FIG. 18 shows the effect of LPS or Cu+2 exposure on TF mRNA levels.

FIG. 19 shows the GSH content in human promyelocytic leukemia cells U937 following treatment with 7-ketocholesterol.

FIG. 20 is a photomicrograph of atheroma (type IV lesion) in proximal left anterior descending coronary artery from a 23-year old man who died of a homicide.

FIG. 21 is a photomicrograph of thick part of atheroma (type IV lesion) in proximal left anterior descending coronary artery from a 19-year-old man who committed suicide.

FIG. 22 shows TF activity over time following treatment with herpes simplex virus-1 (HSV-1), LPS or platelet-derived growth factor (PDGF).

FIG. 23 shows a graphic illustration of the change in TF activity over time for a control cell and a cell harboring a GABP viral genome.

FIG. 24 shows a graphic illustration of the microcompetition effect on the relation between catecholamines and lipolysis.

FIGS. 25-28 show the measure effects norepinephrine, isoprenaline, forskolin, and dibutyryl cyclic AMP on glycerol release in adipocytes from subjects with a family trait of obesity and controls.

FIG. 29 shows the measured relationship between epinephrine infusion and glycerol release in obesity versus lean.

FIGS. 30-31 shows the measured percent change and total glycerol release as a function of plasma epinephrine concentration in obese and lean women.

FIG. 32 shows the percent Rb-null preadipocytes in S phase following five different treatments

FIG. 33 shows a graphic illustration of how microcompetition reduces Rb transcription.

FIG. 34 shows some of the molecules on the surface of DC and T cells participating in their binding.

FIG. 35 shows a graphic illustration of how an increase in either [B7] or [Ag], increases the probability of Th1 vs. Th2 differentiation.

FIG. 36 shows a graphic illustration of the relation between time and TF expression for cells migrating through regions of low, moderate, and high antigen concentrations.

FIG. 37 shows a graphic illustration of the relation between trigger apoptosis, T-cell induced apoptosis and tissue cell damage.

FIG. 38 shows a graphic illustration of the two-peak dynamics.

FIG. 39 shows a graphic illustration of the effect of an excessively slow DC on the two-peaks.

FIG. 40 shows the percent change in β cell apoptosis and percent of islet area following five low-dose streptozotocin injections.

FIG. 41 shows the effect on β cell apoptosis of a single injection of cyclophosphamide to 3 and 12 week old NOD mice and an injection of nicotinamide and cyclophosphamide to 12-week-old mice.

FIG. 42 shows a graphic illustration of the effect of thioredoxin (TRX) over expression on the two-peaks.

FIG. 43 shows a graphic illustration of the effect of DC maturation on the number of cells expressing certain concentrations of tissue factor, antigens and costimulation on their surface.

FIG. 44 shows a graphic illustration of the Barratt-Boyes 2000 experimental configuration.

FIG. 45 shows the effect of treatment on the microcompetition equilibrium.

FIG. 46 shows a schematic illustration of how aberrant GABP expression can be restored.

FIG. 47 shows the effect of sodium butyrate treatment on MT mRNA.

FIG. 48 shows the effect of acarbose treatment on change in body weight over time.

FIG. 49 shows the effect of vanadate treatment on PFK-2 mRNA over time.

FIG. 50 shows the change in HIV-1 DNA and RNA load relative to baseline in 42 antiretroviral naive HIV-1 infected persons treated with either AZT monotherapy, a combination of AZT+ddC or a combination of AZT+ddI over a period of 80 weeks.

FIG. 51 graphically shows the result of a regression analysis with viral DNA level as dependent variable and number of years since seroconversion as independent variable.

### DETAILED DESCRIPTION OF THE INVENTION

A. Introduction of invention

1. Detailed description of New Elements

The following sections present descriptions of elements used in the present invention. Following each definition, one or more exemplary assays are provided to illustrate to one skilled in the art how to use the element. Each assay may include, as its own elements, standard methods in molecular biology, microbiology, cell biology, cell culture, transgenic biology, recombinant DNA, immunology, pharmacology, and toxicology, well known in the art. Details of the standard methods are available further below.

a) Microcompetition Related Elements

(1) Microcompetition

Definition

Assume the DNA sequences DNA₁ and DNA₂ bind the transcription complexes C₁ and C₂, respectively. If C₁ and C₂ include the same transcription factor, DNA₁ and DNA₂ are called “microcompetitors.” A special case of microcompetition is two DNA sequences that bind the same transcription complex.

Notes:

1. Transcription factors include transcription coactivators.

2. Sharing the same environment, such as cell, or chemical mix, is not required to be regarded microcompetitors. For instance, two genes that were shown once to bind the same transcription factor are regarded microcompetitors independent of their actual physical environment. To emphasize such independence, the terminology “susceptible to microcompetition” may be used.

Exemplary Assays

1. If DNA₁ and DNA₂ are endogenous in the cell of interest, assay the transcription factors bound to the DNA sequences (see in “Detailed description of standard protocols” below, the section entitled “Identifying a polypeptide bound to DNA or protein complex”) and compare the two sets of polypeptides. If the two sets include a common transcription factor, DNA₁ and DNA₂ are microcompetitors.

2. In assay 1, if DNA₁ and/or DNA₂ are not endogenous, introduce DNA₁ and/or DNA₂ to the cell by, for instance, transfecting the cell with plasmids carrying DNA₁ and/or DNA₂, infecting the cell with a virus that includes DNA₁ and/or DNA₂, and mutating endogenous DNA to produce a sequence identical to DNA₁ and/or DNA₂.

Notes:

1. Introduction of exogenous DNA₁ and/or DNA₂ is a special case of modifying the cellular copy number of a DNA sequence. Such introduction increases the copy number from zero to a positive number. Generally, copy number may be modified by means such as the ones mentioned above, for instance, transfecting the cell with plasmids carrying a DNA sequence of interest, infecting the cell with a virus that includes the DNA sequence of interest, and mutating endogenous DNA to produce a sequence identical to the DNA sequence of interest.

2. Assume DNA₁ and DNA₂ microcompete for the transcription factor F. Assaying the copy number of at least one of the two sequences, that is, DNA₁ and/or DNA₂, is regarded as assaying microcompetition for F, and observing a change in the copy number of at least one of the two sequences is regarded as identification of modified microcompetition for F.

3. Assume the transcription factor F binds the DNA box DNA_{F}. Consider a specific DNA sequence, DNA₁ that includes a DNA_{F} box, then:

[F·DNA₁]=f([DNA_{F}], [F], F-affinity, F-avidity)

The concentration of F bound to DNA₁ is a function of the DNA_{F} copy number, the concentration of F in the cell, F affinity and avidity to its box. Using f, a change in microcompetition can be defined as a change in [DNA_{F}], and a change in [F·DNA₁] as an effect of such change.

4. Note that under certain conditions (fixed [F], fixed F-affinity, fixed F-avidity, and limiting transcription factor (see below)), there is a “one to one” relation between [F·DNA₁] and [DNA_{F}]. Under such conditions, assaying [F·DNA₁] is regarded assaying microcompetition.

### EXAMPLES

See studies in the section below entitled “Microcompetition with a limiting transcription complex.”

(2) Microavailable

Definition

Let L₁ and L₂ be two molecules. Assume L₁ can take s=(1 . . . n) shapes. Let L_{1,s} denote L₁ in shape s, and let [L_{1,s}] denote concentration of L_{1,s}. If L_{1,s} can bind L₂, an increase (or decrease) in [L_{1,s}] in the environment of L₂ is called “increase (or decrease) in microavailability of L_{1,s} to L₂.” Microavailability of L_{1,s} is denoted ₘₐL_{1,s}. A shape that does not bind L₂ is called “microunavailable to L₂.”

Let s=(1 . . . m) denote the set of all L_{1,s} that can bind L₂. Any increase (or decrease) in the sum of [L_{1,s}] over all s=(1 . . . m) is called “increase (or decrease) in microavailability of L₁ to L₂.” Microavailability of L₁ to L₂ is denoted ₘₐL₁.

Notes:

1. A molecule in a complex is regarded in a different shape relative to the same molecule uncomplexed, or free.

2. Consider an example of an antibody against L_{1,j}, a specific shape of L₁. Assume the antibody binds L_{1,j} in the region contacting L₂. Assume the antibody binds a single region of L_{1,j}, and that antibody binding prevents formation of the L₁·L₂ complex. By binding L_{1,j}, the antibody changes the shape of L₁ from L_{1,j} to L_{1,k}, or from exposed to hidden contact region. Since L_{1,k} does not bind L₂, the decrease in [L_{1,j}] decreases ₘₐL₁, or the microavailability of L₁ to L₂. If, on the other hand, the antibody converts L_{1,j} to L_{1,p}, a shape that also forms the L₁·L₂ complex with the same probability, ₘₐL₁ is fixed. The decrease in [L_{1,j}] is equal to the increase in [L_{1,p}], resulting in a fixed sum of [L_{1,s}] computed over all s that bind L₂.

Exemplary Assays

The following assays identify a change in ₘₐL₁ following treatment.

1. Assay in a biological system (e.g., cell, cell lysate, chemical mixture) the concentrations of all L_{1,s}, where s is a shape that can bind L₂. Apply a treatment to the system which may change L_{1,s}. Following that treatment assay again the concentrations of all L_{1,s}, where s is a shape that can bind L₂. Calculate the sum of [L_{1,s}] over all s, before and after treatment. An increase (or decrease) in this sum indicates an increase (or decrease) in ₘₐL₁.

### EXAMPLES

Antibodies specific for L_{1,s} may be used in immunoprecipitation, Western blot or immunoaffinity to quantify the levels of L_{1,s} before and after treatment.

See also examples below.

(3) Limiting Transcription Factor

Definition

Assume the transcription factor F binds DNA₁. F is called “limiting in respect to DNA₁,” if a decrease in microavailability of F to DNA₁ decreases the concentration of F bound to DNA₁ (“bound F”).

Notes:

1. The definition characterizes “limiting” by the relationship between the concentration of microavailable F and the concentration of F actually bound to DNA₁. According to the definition, “limiting” means a direct relationship between a decrease in microavailable F and a decrease in bound F, and “not limiting” means no such relationship between the two variables. For instance, according to this definition, a decrease in microavailable F with no corresponding change in bound F, means, “not limiting.”

2. Let G₁ denote a DNA sequence of a certain gene. Such DNA sequence may include coding and non-coding regions of a gene, such as exons, introns, promoters, enhancers, or other segments positioned 5′ or 3′ to the coding region. Assume the transcription factor F binds G₁. An assay can measure changes in G₁ mRNA expression instead of changes in the concentration of bound F. Assume F transactivates G₁. Since F is necessary for transcription, a decrease in ₘₐF decreases F·G₁, which, in turn, decreases G₁ transcription. However, an increase in concentration of F bound to G₁ does not necessarily increase transcription if binding of F is necessary but not sufficient for transactivation of G₁.

Exemplary Assays

1. Identify a treatment that reduces ₘₐF by trying different treatments, assaying ₘₐF following each treatment, and choosing a treatment that reduces ₘₐF. Assay the concentration of F bound to DNA₁ (see “Basic protocols”) in a biological system (e.g. cell of interest). Use the identified treatment to reduce ₘₐF. Following treatment assay again the concentration of bound F. A decrease in the concentration of F bound to DNA₁ indicates that F is limiting in respect to DNA₁.

2. Transfect a recombinant expression vector carrying the gene expressing F. Expression of this exogenous F will increase the intracellular concentration of F. Following transfection:

(a) Assay the concentration of F bound to DNA₁. An increase in concentration of bound F indicates that F is limiting in respect to DNA₁.

(b) If DNA₁ is the gene G₁, assay G₁ transcription. An increase in G₁ transcription indicates that F is limiting in respect to G₁ (such an increase in transcription is expected if binding of F to G₁ is sufficient for transactivation).

3. Contact a cell with antibodies that reduce ₘₐF. Following treatment:

(a) Assay the concentration of F bound to DNA. A decrease in concentration of bound F with any antibody concentration indicates that F is limiting in respect to DNA₁.

(b) If DNA₁ is the gene G₁, assay G₁ transcription. A decrease in G₁ transcription with any antibody concentration indicates that F is limiting in respect to G₁.

See Kamei 1996² which used anti-CBP immunoglubulin G (IgG). (Instead of antibodies, some studies used E1A, which, by binding to p300/cbp, also converts the shape from microavailable to microunavailable).

4. Modify the copy number of DNA₂, another DNA sequence, or G₂, another gene, which also bind F (by, for instance, transfecting the cell with DNA₂ or G₂, see above).

(a) Assay the concentration of F bound to DNA₁. A decrease in concentration of F bound to DNA₁ indicates that F is limiting in respect to DNA₁.

(b) If DNA₁ is the gene G₁, assay G₁ transcription. A decrease in G₁ transcription indicates that F is limiting in respect to G₁.

If DNA₁ is the gene G₁, competition with DNA₂ or G₂, which also bind F, reduces the concentration of F bound to G₁ and, therefore, the resulting transactivation of G₁ in any concentration of DNA₂ or G₂. In respect to G₁, binding of F to DNA₂ or G₂ reduces microavailability of F to G₁, since F bound to DNA₂ or G₂ is microunavailable for binding with G₁.

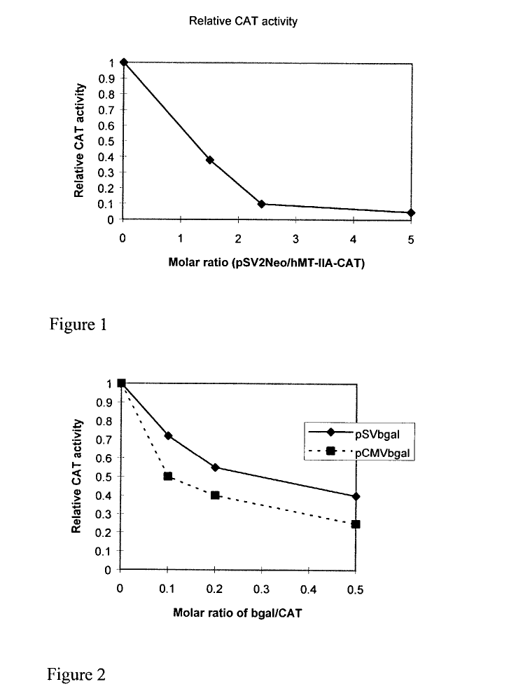

This assay is exemplified in a study reported by Kamei, et al., (1996, ibid). The study used TPA to stimulate transcription from a promoter containing an AP-1 site. AP-1 interacts with CBP. CBP also interacts with a liganded retinoic acid receptor (RAR) and liganded glucocorticoid receptor (GR) (Kamei 1996, ibid, FIG. 1). Both RAR and GR exhibited ligand-dependent repression of TPA stimulated transcription. Induction by TPA was about 80% repressed by treatment with retinoic acid or dexamethasone. In this study, G is the gene controlled by the AP-1 promoter. In respect to this gene, the CBP·liganded-RAR complex is the microunavailable form. An increase in [CBP·liganded-RAR] decreases the concentration of microavailable CBP.

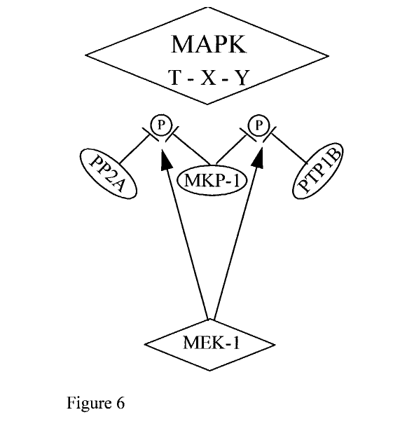

In another exemplary study by Hottiger 1998³, the two genes are HIV-CAT, which binds NF-κB, and GAL4-CAT, which binds the fusion protein GAL4-Stat2(TA). NF-κB binds p300/cbp. The GAL4-Stat2(TA) fusion protein includes the Stat2 transactivation domain that also binds p300/cbp. The study showed a close dependent inhibition of gene activation by the transactivation domain of Stat2 following transfection of a RelA expression vector (Hottiger 1998, ibid, FIG. 6A).

5. Transfect F and modify the copy number of DNA₂, another DNA sequence, or G₂, another gene, which also bind F (by, for instance, transfecting the cell with DNA₂ or G₂, see also above). Following transfection:

(a) Assay concentration of F bound to DNA. Attenuated decrease in concentration of F bound to DNA₁ indicates that F is limiting in respect to DNA₁.

(b) If DNA₁ is the gene G₁, assay G₁ transcription. Attenuated decrease in G₁ transactivation caused by DNA₂ or G₂, indicates that F is limiting in respect to G₁ (see Hottiger 1998, ibid, FIG. 6D).

6. Call the box that binds F the “F-box.” Transfect a cell with DNA₂, another DNA sequence, or G₂ another gene carrying a wild type F-box. Transfect another cell with DNA₂ or G₂ after mutating the F-box in the transfected DNA₂ or G₂.

(a) Assay the concentration of F bound to DNA₁. Attenuated decrease in the concentration of F bound to DNA₁ with the wild type but not the mutated F-box indicates that F is limiting in respect to DNA₁.

(b) If DNA₁ is the gene G₁, assay G₁ transcription. Attenuated decrease in G₁ transactivation with the wild type but not the mutated F-box indicates that F is limiting in respect to G₁.

If DNA₁ is the gene G₁, a mutation in the F-box results in diminished binding of F to DNA₂ or G₂, and an attenuated inhibitory effect on G₁ transactivation. In Kamei 1996 (ibid), mutations in the RAR AF2 domain that inhibit binding of CBP, and other coactivator proteins, abolished AP-1 repression by nuclear receptors.

7. Let t₁ and t₂ be two transcription factors that bind F. Let G₁ and G₂ be two genes transactivated by the t₁·F and t₂·F complexes, respectively.

(a) Transfect a cell of interest with t₁ and assay G₂ transcription. If the increase in [t₁]reduces transcription of G₂, F is limiting in respect to G. Call t₂·F the microavailable shape of F in respect to G₂. The increase in [t₁]increases [t₁·F], which, in turn, reduces [t₂.F]. The decrease in the shape of F microavailable to G₂ reduces transactivation of G₂. In Hottiger 1998 (ibid), t₁ is RelA, t₂ is GAL4-Stat2(TA) and G₂ is GAL4-CAT. See results of the increase in t₁ on G₂ transactivation shown in Hottiger (1998, ibid) FIG. 6A.

(b) Transfect F and assay the concatenation of F bound to G, or transactivation of G. If the increase in F decreases the inhibitory effect of t₁, F is limiting in respect to G (see Hottiger 1998 (ibid), FIG. 6C showing the effect of p300/cbp transfection).

(c) Assay the concentration of t₁, t₂ and F. If t₁ and t₂ have high molar excess compared to F, F is limiting in respect to G (see Hottiger 1998, ibid).

(4) Microcompetition for a Limiting Factor

Definition

Assume DNA₁ and DNA₂ microcompete for the transcription factor F. If F is limiting in respect to DNA₁ and DNA₂, DNA₁ and DNA₂ are called “microcompetitiors for a limiting factor.”

Exemplary Assays

1. The assays 4-7 in the section entitled “Limiting transcription factor” above, can be used to identify microcompetition for a limiting factor.

2. Modify the copy number of DNA₁ and DNA₂ (by, for instance, co-transfecting recombinant vector carrying DNA₁ and DNA₂, see also above).

(a) Assay DNA₁ protection against enzymatic digestion (“DNase footprint assay”). A change in protection indicates microcompetition for a limiting factor.

(b) Assay DNA₁ electrophoretic gel mobility (“electrophoretic mobility shift assay”). A change in mobility indicates microcompetition for a limiting factor.

3. If DNA₁ is a segment of a promoter or enhancer, or can function as a promoter or enhancer, independently, or in combination of other DNA sequences, fuse DNA₁ to a reporter gene such as CAT or LUC. Co-transfect the fused DNA₁ and DNA₂. Assay for expression of the reporter gene. Specifically, assay transactivation of reporter gene following an increase in DNA₂ copy number. A change in transactivation of the reporter gene indicates microcompetition for a limiting factor.

4. A special case is when DNA₁ is the entire cellular genome responsible for normal cell morphology and function. Transfect DNA₂, and assay cell morphology and/or function (such as, binding of extracellular protein, cell replication, cellular oxidative stress, gene transcription, etc). A change in cell morphology and/or function indicates microcompetition for a limiting factor.

Notes:

1. Preferably, following co-transfection of DNA₁ and DNA₂, verify that the polynucleotides do not produce mRNA. If the sequences transcribe mRNA, block translation of proteins with, for instance, an antisense oligonucleotide specific for the exogenous mRNA. Alternatively, verify that the proteins are not involved in binding of F to either sequence. Also, verify that co-transfection does not mutate the F-boxes in DNA₁ and DNA₂, and that the sequences do not change the methylation patterns of their F-boxes. Finally, check that DNA₁ and DNA₂ do not contact each other in the F-box region.

### EXAMPLES

See studies in the section below entitled “Microcompetition with a limiting transcription complex.”

(5) Foreign to

Definition 1

Consider an organism R with standard genome O. Consider Oₛ a segment of O. If a polynucleotide Pn is different from Oₛ for all Oₛ in O, Pn is called “foreign to R.”

Notes:

1. As an example for different organisms consider the list of standard organisms in the PatentIn 3.1 software. The list includes organisms such as, homo sapiens (human), mus musculus (mouse), ovis aries (sheep), and gallus gallus (chicken).

2. A standard genome is the genome shared by most representatives of the same organism.

3. A polynucleotide and DNA sequence (see above) are interchangeable concepts.

4. In multicellular organism, such as humans, the standard genome of the organism is not necessarily found in every cell. The genomes found in sampled cells can vary as a result of somatic mutations, viral integration, etc (see definition below of foreign polynucleotide in a specific cell).

5. Assume Pn expresses the polypeptide Pp. If Pn is foreign to R, then Pp is foreign to R.

6. When the reference organism is evident, instead of the phrase “a polynucleotide foreign to organism R,” the “foreign polynucleotide” phrase might be used.

Exemplary Assays

1. Compare the sequence of Pn with the sequence, or sequences of the published, or self sequenced standard genome of R. If the sequence is not a segment of the standard genome, Pn is foreign to R.

2. Isolate DNA from O (for instance, from a specific cell, or a virus). Try to hybridize Pn to the isolated DNA. If Pn does not hybridize, it is foreign.

Notes:

1. Pn can still be foreign if it hybridizes with DNA from a specific O specimen. Consider, for example, the case of integrated viral genomes. Viral sequences integrated into cellular genomes are foreign. To increase the probability of correct identification, repeat the assay with N>1 specimens of O (for instance, by collecting N cells from different representatives of R). Define the genome of R as all DNA sequences found in all O specimens. Following this definition, integrated sequences, which are only segments of certain O specimens, are identified as foreign. Note that the test is dependent on the N population. For instance, a colony that propagates from a single cell might include a foreign polynucleotide in all daughter cells. Therefore, the N specimens should include genomes (or cells) from different lineages.

2. A polynucleotide can also be identified as potentially foreign if it is found episomally in the nucleus. If the DNA is found in the cytoplasm, it is most likely foreign. Also, a large enough polynucleotide can be identified as foreign if many copies of the polynucleotide can be observed in the nucleus. Finally, if Pn is identical to sequences in genomes of other organisms, such as viruses or bacteria, known to invade R cells, and specifically nuclei of R cells, Pn is likely foreign to R.

Definition 2

Consider an organism R. If a polynucleotide Pn is immunologically foreign to R, Pn is called “foreign to R.”

Notes:

1. In Definition 1, the comparison between O, the genome of the R organism, and Pn is performed logically by the observer. In definition 2, the comparison is performed biologically by the immune system of the organism R.

2. Definition 2 can be generalized to any compound or substance. A compound X is called foreign to organism R, if X is immunologically foreign to R.

Exemplary Assays

1. If the test polynucleotide includes a coding region, incorporate the test polynucleotide in an expressing plasmid and transfer the plasmid into organism R, through, for instance, injection (see DNA-based immunization protocols). An immune response against the expressed polypeptide indicates that the polynucleotide is foreign.

2. Inject the test polynucleotide in R. An immune response against the injected polynucleotide indicates that the test polynucleotide is foreign.

### EXAMPLES

Many viruses, nuclear, such as Epstein-Barr, and cytoplasmic, such as Vaccinia, express proteins which are antigenic and immunogenic in their respective host cells.

Definition 3

Consider an organism R with standard genome O. Consider Oₛ, a segment of O. If a polynucleotide Pn is chemically or physically different than Oₛ for all Oₛ in O, Pn is called “foreign to R.”

Notes:

1. In Definition 3, the observer compares O, the genome of the R organism, with Pn using the molecules chemical or physical characteristics.

Exemplary Assays

In general, many assays in the “Detection of a genetic lesion” section below compare a test polynucleotide and a wild-type polynucleotide. In these assay, let Oₛ be the wild-type polynucleotide and use the assays to identify a foreign polynucleotide. Consider the following examples.

1. Compare the electrophoretic gel mobility of Oₛ and the test polynucleotide. If mobility is different, the polynucleotides are different.

2. Compare the patterns of restriction enzyme cleavage of Oₛ and the test polynucleotide. If the patterns are different, the polynucleotides are different.

3. Compare the patterns of methylation of Oₛ and the test polynucleotide (by, for instance, electrophoretic gel mobility). If the patterns are different, the polynucleotides are different.

Definition 4

Consider an organism R with standard genome O. Let [Pn] denote the copy number of Pn in O. Consider a cell Cellᵢ. Let [Pn]ᵢ denote the copy number of Pn in Cellᵢ. If [Pn]ᵢ>[Pn], Pn is called “foreign to Cellᵢ.”

Note

1. [Pn]ᵢ is the copy number of all Pn in Cellᵢ, from all sources. For instance, [Pn] includes all Pn segments in O, all Pn segments of viral DNA in the cell (if available), all Pn segments of plasmid DNA in the cell (if available), etc.

1. If [Pn]=0, the definition is identical to definition 1 of foreign polynucleotide.

Exemplary Assays

1. Sequence the genome of Cellᵢ. Count the number of time Pn appears in the genome. Compare the result to the number of times Pn appears in the published standard genome. If the number is greater, Pn is foreign to Cellᵢ.

2. Sequence the genome of Cellᵢ and a group of other cells Cellⱼ, . . . Cellⱼ₊ₘ. If [Pn]ᵢ>[Pn]ⱼ= . . . =[Pn]ⱼ₊ₘ, Pn is foreign to Cellᵢ.

(6) Natural to

Definition

Consider an organism R with standard genome O. If a polynucleotide Pn is a fragment of O, Pn is called “natural to R.”

Notes:

1. “Natural to” and “foreign to” are mutually exclusive. A polynucleotide cannot be both foreign and natural to R. If a polynucleotide is natural, it is not foreign to R, and if a polynucleotide is foreign, it is not natural to R.

2. If Pn is a gene natural to R, then, its gene product is also natural to R.

3. The products of a reaction carried out in a cell between gene products natural to the cell, under normal conditions, are natural to the cell. For instance, cellular splicing by factors natural to the cell produce splice products natural to the cell.

Exemplary Assays

1. Compare the sequence of Pn with the sequence, or sequences of the published, or self sequenced standard genome of R. If the sequence is a segment of the standard genome, Pn is natural to R.

2. Isolate DNA from O (for instance, from a specific cell, or a virus). Try to hybridize Pn to the isolated DNA. If Pn hybridizes, it is natural.

Notes:

1. Hybridization with DNA from a specific O specimen of R is not conclusive evidence that Pn is natural to R. Consider, for example, the case of integrated viral genomes. Viral sequences integrated into cellular genomes are foreign. To increase the probability of correct identification, repeat the assay with N>1 specimens of O (for instance, by collecting N cells from different representatives of R). Define the genome of R as all DNA sequences found in all O specimens. Following this definition, integrated sequences that are only segments of certain 0 specimens are identified as foreign. Note that the test is dependent on the N population. For instance, a colony that propagates from a single cell might include a foreign polynucleotide in all daughter cells. Therefore, the N specimens should include genomes (or cells) from different lineages.

(7) Empty Polynucleotide

Definition

Consider the Pn polynucleotide. Consider an organism R with genome O_{R}. Let Pp(Pn), and Pp(O_{R}) denote a gene product (polypeptide) of a Pn or O_{R} gene, respectively. If Pp(Pn)≠Pp(O_{R}) for all Pp(Pn), Pn will be called an “empty polynucleotide” in respect to R.

Notes:

1. A vector is a specific example of a polynucleotide.

2. A vector that includes a non coding polynucleotide natural to R is considered empty in respect to the R. (“natural to” is the opposite of “foreign to.” Note: A natural polynucleotide means, a polynucleotide natural to at least one organism. An artificial polynucleotide means a polynucleotide foreign to all known organisms. A viral enhancer is a natural polynucleotide. A plasmid with a viral enhancer fused to a human gene is artificial.)

3. A vector that includes a coding gene natural to Q, an organism different from R, can still be considered empty in respect to R. For instance, a vector that includes the bacterial chloramphenicol transacetylase (CAT), bacterial neomycin phosphotransferase (neo), or the firefly luciferase (LUC) as reporter genes, but no human coding gene is considered empty in respect to the humans if it does not express a gene natural to humans.

Exemplary Assays

1. Identify all gene products encoded by Pn. Compare to the gene products of O_{R}. If all gene products are different, Pn is considered empty in respect to the R.

### EXAMPLES

pSV2CAT, which expresses the chloramphenicol acethyltransferase (CAT) gene under the control of the SV40 promoter/enhancer, pSV2neo, which expresses the neo gene under the control of the SV40 promoter/enhancer, HSV-neo, which expresses the neomycin-resistance gene under control of the murine Harvey sarcoma virus long terminal repeat (LTR), pZIP-Neo, which expresses the neomycin-resistant gene under control of the Moloney murine leukemia virus long terminal repeat (LTR), are considered empty polynucleotides, or empty vectors, in respect to humans and in respect to the respective virus. See more examples below.

Note: These vectors can be considered as “double” empty, empty in respect to humans, and empty in respect to the respective virus.

(8) Latent Foreign Polynucleotide

Definition

Consider Pn, a polynucleotide foreign to organism R. Pn will be called latent in a Cellᵢ of R if over an extended period of time, either:

1. Pn produces no Pn transcripts.

2. Denote the set of gene products expressed by Pn in Cellᵢ with Cell_{i—}Pp(Pn) and the set of all possible gene products of Pn with All_Pp(Pn), then, Cell_{i—}Pp(Pn)⊂All_Pp(Pn), that is, the set of Pn gene products expressed in Cellᵢ is a subset of all possible Pn gene products.

3. Pn shows limited or no replication.

4. Pn is undetected by the host immune system.

5. Cellᵢ shows no lytic symptoms.

6. R shows no macroscopic symptoms.

Notes:

1. A virus in a host cell is a foreign polynucleotide. According to the definition, a virus is considered latent if, over an extended period of time, it either shows partial expression of its gene products, no viral mRNA, limited or no replication, is undetected by the host immune system, causes no lytic symptoms in the infected cell, or causes no macroscopic symptoms in the host.

2. The above list of characterizations is not exhaustive. The medical literature includes more aspects of latency that can be added to the definition.

Exemplary Assays

1. Introduce, or identify a foreign polynucleotide in a host cell. Assay the polynucleotide replication, or transcription, or mRNA, or gene products over an extended period of time. If the polynucleotide shows limited replication, no transcription, or a limited set of transcripts, the polynucleotide is latent.

2. Introduce, or identify a foreign polynucleotide in a host cell. Assay the cell over an extended period of time, if the cell shows no lytic symptoms, the polynucleotide is latent.

### EXAMPLES

Using PCR, a study (Gonelli 2001⁴) observed persistent presence of viral human herpes virus 7 (HHV-7) DNA in biopsies from 50 patients with chronic gastritis. The study also observed no U14, U17/17, U31, U42 and U89/90, HHV-7 specific transcripts highly expressed during replication. Based on these observations, the study concluded that “gastric tissue represents a site of HHV-7 latent infection and potential reservoir for viral reactivation.” To test the effect of treatment on the establishment of latent herpes simplex virus, type 1 (HSV-1) in sensory neurons, another study (Smith 2001⁵) assays the expression of the latency-associated transcript (LAT), the only region of the viral genome transcribed at high levels during the period of viral latency. A recent review (Young 2000⁶) discusses the limited sets of Epstein-Barr viral (EBV) gene products expressed during the period of viral latency.

(9) Partial Description

Definition

Let Cᵢ be a characteristic of a system. Let the set Ci, i=(1 . . . m) be the set of characteristics providing a complete description of the system. Any subset of Ci, i=(1 . . . m) is called a “partial description” of the system.

Exemplary Assays

1. Chose any set of characteristics describing the system and assay these characteristics.

### EXAMPLES

Assaying blood pressure, blood triglycerides, glucose tolerance, body weight, etc.

(10) Equilibrium

Definition

If a system persists in a state St₀ over time, St₀ is called equilibrium.

Note:

The system related definitions can be modified to accommodate partial descriptions. For example, consider a description of a system which includes only a proper subset of Ci, i=(1 . . . m). If the values measured for the subset of characteristics in St₀ persist over time, the probability that St₀ is an equilibrium is greater than zero. However, since the values are measured only on a subset of Ci, i=(1 . . . m), the probability is less than 1. Overall, an increase in the size of the subset of characteristics increases the probability.

Exemplary Assays

1. Assay the values of the complete (sub) set of the system characteristics. Repeat the assays over time. If the values persist, the system is (probably) in equilibrium.

### EXAMPLES

Regular physicals include standard tests, such as blood count, cholesterol levels, HDL cholesterol, triglycerides, kidney function tests, thyroid function tests, liver function tests, minerals, blood sugar, uric acid, electrolytes, resting electrocardiogram, an exercise treadmill test, vision testing, and audiometry. When the values in these tests remain within a narrow range over time, the medical condition of the subject can be labeled as a probable equilibrium. Other tests performed to identify deviations from equilibrium are mammograms and prostate cancer screenings.

(11) Stable Equilibrium

Definition

Consider an equilibrium E₀. If, after small disturbances, the system always returns to E₀, the equilibrium is called “stable.” If the system moves away from E₀ after small disturbances, the equilibrium is called “unstable.”

Exemplary Assays

1. Take a biological system (e.g., cell, whole organism, etc). Assay a set of characteristics. Verify that the system is in equilibrium, that is, the values of these characteristics persist over time. Apply treatment to the system and assay the set of characteristics again. Repeat assaying over time. If the treatment changed the values of the characteristics, and within a reasonable time the values returned to the original levels, the equilibrium is stable.

(12) Chronic Disease

Definition

Let a healthy biological system be identified with a certain stable equilibrium. A stable equilibrium different from the healthy system equilibrium is called “chronic disease.”

Notes:

1. In chronic disease, in contrast to acute disease, the system does not return to the healthy equilibrium on its own.

Exemplary Assays

1. Take a biological system (e.g., cell, whole organism, etc). Assay a set of characteristics. Compare the results with the values of the same characteristics in healthy controls. If some values deviate from the values of healthy controls, and the values continue to deviate over time, the equilibrium of the system can be characterizes as chronic disease.

### EXAMPLES

High blood pressure, high body weight, hyperglycemia, etc.

(13) Disruption

Definition

Let a healthy biological system be identified with a certain stable equilibrium. Any exogenous event that produces a new stable equilibrium is called “disruption.”

Notes:

1. Using the above definitions it can be said that a disruption is an exogenous event that produces a chronic disease.

2. A disruption is a disturbance with a persisting effect.

Exemplary Assays

1. Take a biological system (e.g., cell, whole organism, etc). Assay a set of characteristics. Compare the results with the values of the same characteristics in healthy controls. Verify that the system is in healthy equilibrium. Apply a chosen treatment to the system. Following treatment, assay the same characteristics again. If some values deviate from the values of healthy controls, continue to assay these characteristics over time. If the values continue to deviate over time, the treatment produced a chronic disease, and, therefore, can be considered a disruption.

### EXAMPLES

Genetic knockout, carcinogens, infection with persistent viruses (e.g., HIV, EBV), etc.

(14) Foreign Polynucleotide-Type Disruption (Cause of Disruption)

Definition

Let Pp be a polypeptide. Assume microcompetition with a foreign polynucleotide Pn directly, or indirectly reduces (or increases) Pp bioactivity. A disruption that directly, or indirectly reduces (or increases) Pp bioactivity is called “foreign polynucleotide-type disruption.”

Notes:

1. The first “indirectly” in the definition means that Pp can be downstream from the gene microcompeting with Pn. The second “indirectly” means that Pp can be downstream from the gene, or polypeptide, directly affected by the exogenous event. According to the definition, if both microcompetition with a foreign polynucleotide and an exogenous event increase, or both decrease bioactivity of Pp, the exogenous event can be considered as a foreign polynucleotide-type disruption.

2. Microcompetition with a foreign polynucleotide is a special case of foreign polynucleotide-type disruption.

3. Treatment is a special case of an exogenous event.

4. A foreign polynucleotide-type disruption can first affect a gene or a polypeptide. For instance, a mutation is an effect on a gene. Excessive protein phosphorylation is an effect on a polypeptide.

Exemplary Assays

1. Take a biological system (e.g., cell, whole organism, etc). Assay a set of characteristics. Compare the results with the values of the same characteristics in healthy controls to verify that the system is in a healthy equilibrium. Modify the copy number of Pn, a polynucleotide of interest (by, for instance, transfection, infection, mutation, etc, see above). Identify a gene with modified expression. Assume the assays show decreased expression of G. Take another specimen of the system in healthy equilibrium and apply a chosen treatment to the healthy specimen. Following treatment, assay G expression. Continue to assay G expression over time. If G expression is persistently decreased, the exogenous event can be considered a foreign polynucleotide-type disruption.

### EXAMPLES

A mutation in the leptin receptor, a mutation in the leptin gene, etc (see more examples below).

(15) Disrupted (Gene, Polypeptide) (Result of Disruption)

Definition

Let Pp be a polypeptide. If a foreign polynucleotide-type disruption modifies (reduces or increases) Pp bioactivity, Pp and the gene encoding Pp are called “disrupted.”

Notes:

1. Pp can be downstream from G, the microcompeted gene.

Exemplary Assays

1. Take a biological system (e.g., cell, whole organism, etc). Modify the copy number of Pn, a polynucleotide of interest, (by, from instance, transfection, infection, mutation, etc, see above). Assay bioactivity of genes and polypeptides in the treated system and controls to identify genes and polypeptides with modified bioactivity relative to controls. These genes and polypeptides are disrupted.

### EXAMPLES

See studies in the section below entitled “Microcompetition with a limiting transcription complex.” See also all GABP regulated genes below.

(16) Disrupted Pathway

Definition

Let the polypeptide Ppₓ be disrupted. A polypeptide Ppᵢ which functions downstream or upstream of Ppₓ, and the gene encoding Ppᵢ, are considered a polypeptide and gene, respectively, in a Ppₓ “disrupted pathway.”

Exemplary Assays

1. Take a biological system (e.g., cell, whole organism, etc). Apply a treatment to the system that modifies Ppᵢ bioactivity. Assay Ppₓ bioactivity. If the bioactivity of Ppₓ changed, Ppᵢ is in a Ppₓ disrupted pathway.

2. Take a biological system (e.g., cell, whole organism, etc). Apply a treatment to the system that modifies Ppₓ bioactivity. Assay Ppᵢ bioactivity. If the bioactivity of Pp changed, Ppᵢ is in a Ppₓ disrupted pathway.

### EXAMPLES

See examples below.

(17) Disruptive Pathway

Definition

Consider a polypeptide Ppₖ and a foreign polynucleotide Pn. If a change in bioactivity of PPₖ increases or decreases Pn copy number, PPₖ and the gene encoding PPₖ are considered a polypeptide and a gene in a Pn “disruptive pathway.”

Notes:

Consider, as an example, microcompetition between a cell and a viral polynucleotide, including the entire viral genome. Ppₖ can be any viral or cellular protein that increase or decreases viral replication.

Exemplary Assays

1. Take a biological system (e.g., cell, whole organism, etc). Apply a treatment to the system that modifies PPₖ bioactivity, for instance, by increasing expression of a foreign or cellular gene encoding Ppₖ. Assay Pn copy number. If the copy number changed, Ppₖ and the gene encoding Ppₖ, are in a Pn disruptive pathway.

### EXAMPLES

Consider a GABP virus. The viral proteins that increase viral replication increase the copy number of viral N-boxes in infected cells. According to the definition, these proteins belong to a disruptive pathway. See specific examples below.

b) p300/cbp Related Elements

(1) p300/cbp

Definition

A member of the p300/cAMP response element (CREB) binding protein (CBP) family of proteins is called p300/cbp.

Notes:

1. For reviews on the p300/cbp family of proteins, see, for instance, Vo 2001⁷, Blobel 2000⁸, Goodman 2000⁹, Hottiger 2000¹⁰, Giordano 1999¹¹, Eckner 1996¹².

2. CREB binding protein (CBP, or CREBBP) is also called RTS, Rubinstein-Taybi syndrome protein, and RSTS.

3. See sequences of p300/cbp genes and p300/cbp proteins in the List of Sequences below.

Exemplary Assays

1. p300/cbp may be identified using antibodies in binding assays, oligonucleotide probes in hybridization assays, transcription factors such as GABP, NF-κB, E1A in binding assays, etc. (see protocols for binding and hybridization assays below).

### EXAMPLES

See examples of below.

(2) p300/cbp Polynucleotide

Definition

Assume the polynucleotide Pn binds the transcription complex C. If C contains p300/cbp, Pn is called “p300/cbp polynucleotide.”

Exemplary Assays

1. Take a cell of interest. Modify the copy number of Pn (by, for instance, transfection, infection, mutation, etc, see also above). Use assays described in the section entitled “Identifying a polypeptide bound to DNA or protein complexes,” or similar assays, to test if the protein-Pn complexes contain p300/cbp.

2. See more assays below.

### EXAMPLES

See below in p300/cbp virus and p300/cbp regulated gene.

(3) p300/cbp Factor

Definition

Assume the transcription factor F binds the complex C. If C contains p300/cbp, F is called “p300/cbp factor.”

Exemplary Assays

1. Use assays describe in the section entitled “Identifying a polypeptide bound to DNA or protein complexes,” or similar assays, to test whether the complexes which contain F also contain p300/cbp.

### EXAMPLES

The following table lists some cellular and viral p300/cbp factors.

| p300/cbp | Gene | | |
|---|---|---|---|
| factor | symbol | Other names | References |
| Cellular | | | |
| AML1 | RUNX1 | acute myeloid leukemia 1 | Kitabayashi |
| | CBFA2 | protein (AML1); core-binding | 1998¹³ |
| | AML1 | factor α2 subunit (CBFα2); | |
| | | oncogene AML-1; Polyoma- | |
| | | virus enhancer binding protein | |
| | | 2αB subunit (PEBP2αB); | |
| | | PEA2αB; SL3-3 enhancer | |
| | | factor 1, αB subunit; | |
| | | SL3/AKV core-binding factor | |
| | | αB subunit; SEF1; runt- | |
| | | related transcription factor 1; | |
| | | RUNX1; CBFA2 | |
| A-Myb | MYBL1 | Myb-related protein A; v-myb | Facchinetti |
| | AMYB | avian myeloblastosis viral | 1997¹⁴ |
| | | oncogene homolog-like 1 | |
| ATF1 | ATF1 | activating transcription factor | Goodman 2000 |
| | TREB36 | 1 (ATF1); TREB36 protein; | (ibid) |
| | | cAMP-dependent transcription | |
| | | factor ATF-1 | |
| ATF2 | ATF2 | Activating transcription factor | Goodman 2000 |
| | CREB2 | 2 (ATF2); cAMP response | (ibid), |
| | CREBP1 | element binding protein 1 | Duyndam |
| | | (CRE-BP1); HB16; cAMP- | 1999¹⁵ |
| | | dependent transcription factor | |
| | | ATF-2; TREB7; CREB2 | |
| ATF4 | ATF4 | activating transcription factor | Goodman 2000 |
| | CREB2 | 4 (ATF4); DNA-binding | (ibid), Yukawa |
| | TAXREB67 | protein TAXREB67; tax- | 1999¹⁶ |
| | | responsive enhancer element | |
| | | B67 (TAXREB67); TXREB; | |
| | | cAMP response element- | |
| | | binding protein 2 (CREB2); | |
| | | cAMP-dependent transcription | |
| | | factor ATF-4; CCAAT/ | |
| | | enhancer binding protein | |
| | | related activating transcription | |
| | | factor (mouse); ApCREB2 | |
| | | (Aplysia) | |
| BRCA1 | BRCA1 | Breast cancer type 1 | Goodman 2000 |
| | PSCP | susceptibility protein | (ibid) |
| | | (BRCA1) | |
| C/EBPβ | CEBPB | CCAAT/enhancer binding | Goodman 2000 |
| | TCF5 | protein β (C/EBPβ); nuclear | (ibid), Mink |
| | | factor NF-IL6 (NFIL6); | 1997¹⁷ |
| | | transcription factor 5; CRP2; | |
| | | LAP; IL6DBP; CEBPB; | |
| | | TCF5 | |
| c-Fos | FOS | proto-oncogene protein c-fos; | Goodman 2000 |
| | G0S7 | cellular oncogene fos; G0/G1 | (ibid), Sato |
| | | switch regulatory protein 7; | 1997 (ibid) |
| | | v-fos FBJ murine osteo- | |
| | | sarcoma viral oncogene | |
| | | homolog; FOS; G0S7 | |
| C2TA | MHC2TA | MHC class II transactivator; | Goodman 2000 |
| | CIITA | MHC2TA; CIITA | (ibid), Sisk |
| | C2TA | | 2000 (ibid) |
| AP1 | JUN | transcription factor AP-1; | Goodman 2000 |
| | | proto-oncogene c-Jun (c-Jun); | (ibid), Hottiger |
| | | p39; v-jun avian sarcoma | 2000 (ibid) |
| | | virus 17 oncogene homolog | |
| c-Myb | MYB | Myb proto-oncogene protein; | Goodman 2000 |
| | | MYB; v-myb avian | (ibid), Hottiger |
| | | myeloblastosis viral oncogene | 2000 (ibid) |
| | | homolog | |
| CREB | CREB1 | cAMP-respone-element- | Hottiger 2000 |
| | | binding protein (CREB) | (ibid) |
| CRX | CRX | cone-rod homeobox (CRX); | Yanagi 2000¹⁸ |
| | CORD2 | CRD; cone rod dystrophy 2 | |
| | CRD | (CORD2) | |
| CID | CI-D | cubitus interruptus dominant | Goodman 2000 |
| | | (CID) | (ibid) |
| DBP | DBP | D-site binding protein (DBP); | Lamprecht |
| | | albumin D box-binding | 1999¹⁹ |
| | | protein; D site of albumin | |
| | | promoter (albumin D-box) | |
| | | binding protein; TAXREB302 | |
| E2F1 | E2F1 | retinoblastoma binding protein | Goodman 2000 |
| | RBBP3 | 3 (RBBP-3); PRB-binding | (ibid), Marzio |
| | | protein E2F-1; PBR3; | 2000²⁰ |
| | | retinoblastoma-associated | |
| | | protein 1 (RBAP-1) | |
| E2F2 | E2F2 | transcription factor E2F2 | Marzio 2000 |
| | | | (ibid) |
| E2F3 | E2F3 | transcription factor E2F3; | Marzio 2000 |
| | KIAA0075 | KIAA0075 | (ibid) |
| Egr1 | EGR1 | early-growth response factor-1 | Silverman |
| | ZNF225 | (Egr1); Krox-24 protein; | 1998²¹ |
| | | ZIF268; nerve growth factor- | |
| | | induced protein A; NGFI-A; | |
| | | transcription factor ETR103; | |
| | | zinc finger protein 225 | |
| | | (ZNF225); AT225; TIS8; | |
| | | G0S30; ZIF-268 | |
| ELK1 | ELK1 | ets-domain protein ELK-1 | Hottiger 2000 |
| | | | (ibid) |
| ERα | ESRI | estrogen receptor α (ERα); | Kim 2001²², |
| | NR3A1 | estrogen receptor 1; estradiol | Wang 2001²³, |
| | ESR | receptor | Speir 2000²⁴, |
| | | | Hottiger 2000 |
| | | | (ibid) |
| ERβ | ESR2 | estrogen receptor β; ESR2; | Kobayashi |
| | NR3A2 | NR3A2; ESTRB | 2000²⁵ |
| | ESTRB | | |
| ER81 | | Ets translocation variant 1 | Papoutso- |
| | | (ETV1) | poulou 2000²⁶ |
| Ets1 | ETS1 | C-ets-1 protein; v-ets avian | Goodman 2000 |
| | | erythroblastosis virus E2 | (ibid), |
| | | oncogene homolog 1; p54 | Jayaraman |
| | | | 1999²⁷ |
| Ets2 | ETS2 | C-ets-2 protein; human | Jayaraman |
| | | erythroblastosis virus | 1999 (ibid) |
| | | oncogene homolog 2; v-ets | |
| | | avian erythroblastosis virus | |
| | | E2 oncogene homolog 2 | |
| GABPα | GABPA | GA binding protein, α subunit | Bannert 1999²⁸ |
| | E4TF1A | (GABPA); GABP-alpha | |
| | | subunit; transcription factor | |
| | | E4TF1-60; nuclear respiratory | |
| | | factor-2 subunit alpha | |
| | | (NRF-2A) | |
| GABPβ1 | GABPB1 | GA binding protein beta-1 | Bannert 1999 |
| | GABPB | chain (GABPB1); GABP- | (ibid) |
| | E4TF1B | beta-1 subunit; transcription | |
| | | factor E4TF1-53; nuclear | |
| | | respiratory factor-2 subunit | |
| | | beta 2 (NRF-2B) | |
| GABPβ2 | GABPB1 | GA binding protein beta-2 | Bannert 1999 |
| | GABPB | chain (GABPB2); GABP- | (ibid) |
| | E4TF1B | beta-2 subunit; transcription | |
| | | factor E4TF1-47 | |
| GATA1 | GATA1 | globin transcription factor 1; | Goodman 2000 |
| | GF1 | GATA-binding protein 1 | (ibid) |
| | ERYF1 | erythroid transcription factor; | |
| | NFE1 | ERYF1; GF1; NF-E1 | |
| Gli3 | GLI3 | zinc finger protein GLI3; | Goodman 2000 |
| | | PAP-A; GCPS; GLI-Kruppel | (ibid) |
| | | family member GLI3 (Greig | |
| | | cephalopolysyndactyly | |
| | | syndrome); Pallister-Hall | |
| | | syndrome (PHS) | |
| GR | NR3C1 | glucocorticoid receptor (GR); | Pfitzner 1998 |
| | GRL | nuclear receptor subfamily 3, | (ibid), Hottiger |
| | GCR | group C, member 1 (NR3C1); | 2000 (ibid) |
| | | GRL | |
| HIF1α | HIF1A | hypoxia-inducible factor-1 α | Goodman 2000 |
| | | (HIF1α); ARNT interacting | (ibid), |
| | | protein; member of PAS | Bhattacharya |
| | | protein 1; MOP1 | 1999²⁹, Kallio |
| | | | 1998³⁰, Ema |
| | | | 1999³¹, |
| | | | Hottiger 2000 |
| | | | (ibid) |
| HNF4α | HNF4A | heaptocyte nulcear factor-1 α; | Goodman 2000 |
| | NR2A1 | HNF-4-α; transcription factor | (ibid), |
| | TCF14 | HNF-4; transcription factor | Soutoglou |
| | HNF4 | 14; MODY; maturity onset | 2000³² |
| | | diabetes of the young 1; | |
| | | MODY1; HNF4A; NR2A1; | |
| | | TCF14; HNF | |
| IRF-3 | IRF3 | interferon regulatory factor-3 | Goodman 2000 |
| | | (IRF-3) | (ibid), |
| | | | Yoneyama |
| | | | 1998³³ |
| JunB | JUNB | transcription factor JunB; | Goodman 2000 |
| | | proto-oncogene JunB | (ibid) |
| Mdm2 | MDM2 | mouse double minute 2; | Goodman 2000 |
| | | human homolog of p53- | (ibid) |
| | | binding protein (Mdm2); | |
| | | ubiquitin-protein ligase E3 | |
| | | Mdm2; EC 6.3.2.-; p53- | |
| | | binding protein Mdm2; | |
| | | oncoprotein Mdm2; double | |
| | | minute 2 protein; Hdm2 | |
| MEF2C | MEF2C | myocyte enhancer factor 2C | Sartorelli |
| | | (MEF2C); myocyte-specific | 1997 (ibid) |
| | | enhancer factor 2C; MADS | |
| | | box transcription enhancer | |
| | | factor 2 polypeptide C | |
| Mi | MITF | microphthalmia-associated | Goodman 2000 |
| | | transcription factor | (ibid), Sato |
| | | | 1997³⁴ |
| MyoD | MYOD1M | myoblast determination | Yuan 1996 |
| | YF3 | protein 1 (MyoD); myogenic | Ref, Sartorelli |
| | | factor MYF-3; myogenic | 1997³⁵ |
| | | factor 3; PUM | |
| NF-AT1 | NFAT1 | nuclear factor of activated T | Garcia- |
| | NFATC2 | cells, cytoplasmic 2; T cell | Rodriguez |
| | NFATP | transcription factor NFAT1; | 1998³⁶, Sisk |
| | | NFAT pre-existing subunit; | 2000³⁷ |
| | | NF-ATp | |
| NF-YB | NFYB | NF-Y protein chain B | Li 1998³⁸, |
| | HAP3 | (NF-YB); nuclear | Faniello 1999³⁹ |
| | | transcription factor Y subunit | |
| | | beta; α-CP1, CP1; CCAAT- | |
| | | binding transcription factor | |
| | | subunit A (CBF-A); CAAT- | |
| | | box DNA binding protein | |
| | | subunit B | |
| NF-YA | NFYA | NF-Y protein chain A | Li 1998 (ibid) |
| | HAP2 | (NF-YA); CCAAT-binding | |
| | | transcription factor subunit B | |
| | | (CBF-B); CAAT-box DNA | |
| | | binding protein subunit A; | |
| | | nuclear transcription factor | |
| | | Y α | |
| RelA | RELA | NF-κB RelA, transcription | Hottiger 1998 |
| | NFKB3 | factor p65; nuclear factor | (ibid), |
| | | NF-kappa-B, p65 subunit; v- | Gerritsen |
| | | rel avian reticuloendotheliosis | 1997⁴⁰, Speir |
| | | viral oncogene homolog A; | 2000 (ibid), |
| | | nuclear factor of kappa light | Hottiger 2000 |
| | | polypeptide gene enhancer in | (ibid) |
| | | B-cells 3 (p65) | |
| P/CAF | P/CAF | p300/cbp-associated factor | Goodman 2000 |
| | | | (ibid) |
| p/CIP | TRAM-1 | p300/cbp interacting protein | Goodman 2000 |
| | NCOA3 | (p/CIP); thyroid hormone | (ibid) |
| | AIB1 | receptor activator molecule; | |
| | | DJ1049g16.2; nuclear | |
| | | receptor coactivator 3 (thyroid | |
| | | hormone receptor activator | |
| | | molecule TRAM-1; receptor- | |
| | | associated coactivator RAC3; | |
| | | amplified in breast cancer | |
| | | AIB1; ACTR | |
| PPARγ | PPARG | peroxisome proliferator | Iannone |
| | NR1C3 | activated receptor γ (PPARG); | 2001⁴¹, |
| | | PPAR-gamma; PPARG1; | Kodera 2000⁴² |
| | | PPARG2 | |
| MRG1 | CITED2 | Cbp/p300-interacting trans- | Bhattacharya |
| | MRG1 | activator 2; MSG-related | 1999 (ibid), |
| | | protein 1; melanocyte-specific | Han 2001⁴³ |
| | | gene 1; MRG1 protein | |
| p45 | NFE2 | nuclear factor, erythroid- | Goodman 2000 |
| NF-E2 | | derived 2 45 kDa subunit; | (ibid) |
| | | NF-E2 45 kDa subunit (p45 | |
| | | NF-E2); leucine zipper protein | |
| | | NF-E2 | |
| p53 | TP53 | cellular tumor antigen p53; | Goodman 2000 |
| | P53 | tumor suppressor p53;, | (ibid), |
| | | phosphoprotein p53; Li- | Avantaggiati |
| | | Fraumeni syndrome | 1997⁴⁴ Van |
| | | | Order 1999⁴⁵, |
| | | | Hottiger 2000 |
| | | | (ibid) |
| p73 | TP73 | tumor protein p73; p53-like | Goodman 2000 |
| | P73 | transcription factor; p53- | (ibid) |
| | | related protein | |
| Pit-1 | POU1F1 | pituitary-specific positive | Goodman 2000 |
| | PIT1 | transcription factor 1; PIT-1; | (ibid) |
| | GHF1 | growth hormone factor 1, | |
| | | GHF-1; POU domain, class 1, | |
| | | transcription factor 1 | |
| RSK1 | RPS6KA1 | 90-kDA ribosomal S6 kinase, | Goodman 2000 |
| | RSK1 | ribosomal protein S6 kinase | (ibid), Hottiger |
| | | alpha 1; EC 2.7.1.-; S6K- | 2000 (ibid) |
| | | alpha 1; 90 kDa ribosomal | |
| | | protein S6 kinase 1; p90- | |
| | | RSK1;, ribosomal S6 kinase | |
| | | 1; RSK-1; pp90RSK1; HU-1 | |
| RSK3 | RPS6KA2 | Ribosomal protein S6 kinase | Hottiger 2000 |
| | RSK3 | alpha 2; EC 2.7.1.-; S6K- | (ibid) |
| | | alpha 2; 90 kDa ribosomal | |
| | | protein S6 kinase 2;, p90-RSK | |
| | | 2; ribosomal S6 kinase 3; | |
| | | RSK-3; pp90RSK3; HU-2 | |
| RSK2 | RPS6KA3 | ribosomal protein S6 kinase | Hottiger 2000 |
| | RSK2 | alpha 3; EC 2.7.1.-; S6K- | (ibid) |
| | ISPK1 | alpha 3; 90 kDa ribosomal | |
| | | protein S6 kinase 3; p90-RSK | |
| | | 3; ribosomal S6 kinase 2; | |
| | | RSK-2; pp90RSK2; Insulin- | |
| | | stimulated protein kinase 1; | |
| | | ISPK-1; HU-2;, HU-3 | |
| RARγ | RARG | retinoic acid receptor γ | Hottiger 2000 |
| | NR1B3 | (RARγ); retinoic acid receptor | (ibid), Yang |
| | | gamma-1, RAR-gamma-1; | 2001⁴⁶ |
| | | RARC; retinoic acid receptor | |
| | | gamma-2; RAR-gamma-2 | |
| RNA | DDX9 | ATP-dependent RNA helicase | Goodman 2000 |
| helicase | NDH2 | A; nuclear DNA helicase II | (ibid) |
| A | | (NDH II); DEAD-box protein | |
| | | 9; leukophysin (LKP) | |
| RXRα | RXRA | retinoic acid receptor RXR-α | Goodman 2000 |
| | NR2B1 | | (ibid), Yang |
| | | | 2001 (ibid) |
| ELK4 | ELK4 | ETS-domain protein ELK-4; | Goodman 2000 |
| | SAP1 | serum response factor | (ibid), Hottiger |
| | | accessory protein 1 (SAP-1); | 2000 (ibid) |
| | | SRF accessory protein 1 | |
| SF-1 | NR5A1 | steroidogenic factor 1 (STF-1, | Goodman 2000 |
| | FTZF1 | SF-1); steroid hormone | (ibid) |
| | AD4BP | receptor AD4BP; Fushi tarazu | |
| | SF1 | factor (Drosophila) homolog | |
| | | 1; FTZ1; ELP; NR5A1 | |
| | | (nuclear receptor subfamily 5, | |
| | | group A, member 1) | |
| Smad3 | MADH3 | mothers against decapenta- | Goodman 2000 |
| | SMAD3 | plegic (Drosophila) homolog | (ibid), |
| | MAD3 | 3 (SMAD 3); mothers against | Janknecht |
| | | DPP homolog 3; Mad3; | 1998⁴⁷, Feng |
| | | hMAD-3; mMad3; JV15-2; | 1998⁴⁸, |
| | | hSMAD3 | Pouponnot |
| | | | 1998 (ibid) |
| Smad4 | MADH4 | mothers against decapenta- | de |
| | SMAD4 | plegic (Drosophila) homolog | Caestecker⁴⁹, |
| | DPC4 | 4 (SMAD 4); mothers against | Pouponnot |
| | | DPP homolog 4; deletion | 1998 (ibid) |
| | | target in pancreatic carcinoma | |
| | | 4, hSMAD4 | |
| Smad1 | MADH1 | mothers against decapenta- | Pearson |
| | SMAD1 | plegic (Drosophila) homolog | 1999⁵⁰, |
| | MADR1 | 1 (SMAD 1); mothers against | Pouponnot |
| | BSP1 | DPP homolog 1; Mad-related | 1998⁵¹ |
| | | protein 1; transforming | |
| | | growth factor-beta signaling | |
| | | protein-1; BSP-1; hSMAD1; | |
| | | JV4-1 | |
| Smad2 | MADH2 | mothers against decapenta- | Pouponnot |
| | SMAD2 | plegic (Drosophila) homolog | 1998 (ibid) |
| | MADR2 | 2 (SMAD 2); mothers against | |
| | | DPP homolog 2; Mad-related | |
| | | protein 2; hMAD-2; JV18-1; | |
| | | hSMAD2 | |
| SRC-1 | SRC1 | steroid receptor coactivtor-1 | Goodman 2000 |
| | NCOA1 | (SRC-1); F-SRC-1; nuclear | (ibid), Hottiger |
| | | receptor coactivator 1 | 2000 (ibid) |
| | | (NCoA-1); SRC1 | |
| SREBP1 | SREBF1 | sterol regulatory element | Goodman 2000 |
| | SREBP1 | binding protein-1 (SREBP-1); | (ibid), Oliner |
| | | sterol regulatory element- | 1996⁵² |
| | | binding transcription factor 1 | |
| SREBP2 | SREBF2 | sterol regulatory element | Goodman 2000 |
| | SREBP2 | binding protein-2 (SREBP-2); | (ibid), Oliner |
| | | sterol regulatory element- | 1996 (ibid) |
| | | binding transcription factor 2 | |
| Stat-1 | STAT 1 | signal transducer and activator | Goodman 2000 |
| | | or transcription-1 α/β; | (ibid), Paulson |
| | | transcription factor ISGF-3 | 1999⁵³, |
| | | components p91/p84; signal | Hottiger 1998 |
| | | transducer and activator of | (ibid), Gingras |
| | | transcription 1, 91 kD | 1999 (ibid), |
| | | (STAT91) | Zhang 1996⁵⁴ |
| Stat-2 | STAT2 | signal transducer and activator | Goodman 2000 |
| | | or transcription-2 (STAT2);; | (ibid), Paulson |
| | | signal transducer and | 1999 (ibid), |
| | | activator of transcription 2, | Hottiger 1998 |
| | | 113 kD (STAT113); p113 | (ibid), Gringras |
| | | | 1999 (ibid), |
| | | | Bhattacharya |
| | | | 1996⁵⁵, |
| | | | Hottiger 2000 |
| | | | (ibid) |
| Stat-3 | STAT3 | signal transducer and activator | Paulson 1999 |
| | APRF | or transcription-3; acute- | (ibid), Hottiger |
| | | phase response factor | 1998 (ibid) |
| Stat-4 | STAT4 | signal transducer and activator | Paulson 1999 |
| | | or transcription-4 | (ibid) |
| Stat-5 | STAT5 | signal transducer and activator | Paulson 1999 |
| | STAT5A | or transcription-5A | (ibid) check, |
| | STAT5B | (STAT5A); MGF; signal | Gingras 1999 |
| | | transducer and activator or | (ibid), Pfitzner |
| | | transcription-5B (STAT5B); | 1998⁵⁶ |
| | | STAT5 | |
| Stat-6 | STAT6 | signal transducer and activator | Paulson 1999 |
| | | or transcription-6 (STAT6); | (ibid) check, |
| | | IL-4 Stat; D12S1644 | Gingras 1999⁵⁷ |
| TAL1 | TAL1 | T-cell acute lymphocytic | Goodman 2000 |
| | SCL | leukemia-1 protein; TAL-1 | (ibid) |
| | TCL5 | protein; STEM cell protein; | |
| | | T-cell leukemia/lymphoma-5 | |
| | | protein | |
| TBP | TBP | TATA box binding protein | Goodman 2000 |
| | TFIID | (TBP); transcription initiation | (ibid) |
| | TF2D | factor TFIID; TATA-box | |
| | | factor; TATA sequence- | |
| | | binding protein; SCA17; | |
| | | GTF2D1; HGNC:15735; | |
| | | GTF2D | |
| TFIIB | TFIIB | transcription factor IIB | Goodman 2000 |
| | TF2B | (TFIIB, TF2B); transcription | (ibid), Hottiger |
| | GTF2B | initiation factor IIB; general | 2000 (ibid) |
| | | transcription factor IIB | |
| | | (GTFIIB, GTF2B) | |
| THRA | THRA | thyroid hormone receptor α | Hottiger 2000 |
| | NR1A1 | (THRA); c-erbA-alpha; | (ibid) |
| | THRA1 | c-erbA-1; EAR-7; EAR7; | |
| | ERBA1 | AR7; avian erythroblastic | |
| | | leukemia viral (v-erb-a) | |
| | | oncogene homolog; ERBA; | |
| | | THRA1; THRA2; THRA3; | |
| | | EAR-7.1/EAR-7.2 | |
| THRB | THRB | thyroid hormone receptor α1 | Hottiger 2000 |
| | NR1A2 | (THRB); thyroid hormone | (ibid) |
| | THR1 | receptor, beta; avian | |
| | ERBA2 | erythroblastic leukemia viral | |
| | | (v-erb-a) oncogene homolog | |
| | | 2; THRB1; THRB2; ERBA2; | |
| | | NR1A2; thyroid hormone | |
| | | receptor β2 (THRB) | |
| Twist | TWIST | Twist related protein; H-twist; | Goodman 2000 |
| | | acrocephalosyndactyly 3 | (ibid), |
| | | (Saethre-Chotzen syndrome); | Hamamori |
| | | twist (Drosophila) homolog; | 1999⁵⁸ |
| | | acrocephalosyndactyly 3 | |
| | | (ACS3) | |
| YY1 | YY1 | Ying Yang 1 (YY1); | Goodman 2000 |
| | | transcriptional repressor | (ibid) |
| | | protein YY1; delta | |
| | | transcription factor; NF-E1; | |
| | | UCRBP; CF1; Yin Yang 1; | |
| | | DELTA; YY1 transcription | |
| | | factor | |
| Viral | | | |
| E1A | | | Goodman 2000 |
| | | | (ibid), Hottiger |
| | | | 2000 (ibid) |
| EBNA2 | | EBV | Goodman 2000 |
| | | | (ibid) |
| Py LT | | polyomavirus large T antigen | Goodman 2000 |
| | | | (ibid) |
| SV40 LT | | simian virus 40 large T | Goodman 2000 |
| | | antigen, TAg | (ibid), Hottiger |
| | | | 2000 (ibid) |
| HPV E2 | | human papillomavirus E2 | Goodman 2000 |
| | | | (ibid) |
| HPV E6 | | human papillomavirus E6 | Goodman 2000 |
| | | | (ibid), Hottiger |
| | | | 2000 (ibid) |
| Tat | | HIV-1 | Goodman 2000 |
| | | | (ibid), Hottiger |
| | | | 2000 (ibid) |
| Tax | | Human T-cell leukemia virus | Goodman 2000 |
| | | type 1 | (ibid), Hottiger |
| | | | 2000 (ibid) |
| Bacterial | | | |
| JMY | | H pylori | Goodman 2000 |
| | | (cag) | (ibid) |

The two major lists are from reviews by Goodman and Smolik (2000, ibid) and Hottiger and Nabel (2000, ibid).

Mutations in some of these p300 factors are currently associated with chronic diseases, for instance, HNF4A with MODY, ESR1 with breast cancer and bronchial asthma, GR with cortisol resistance, etc. Consider the following definition.

(4) p300/cbp Regulated (Gene, Polypeptide)

Definition

Assume the gene G is transactivated, or suppressed by the transcription complex C. If C contains p300/cbp, the gene G, and the polypeptide encoded by G, are called “p300/cbp regulated.”

Exemplary Assays

1. Co-transfect a cell with the gene promoter fused to a reporter gene, such as CAT or LUC, and a vector expressing p300/cbp. Assay reporter gene expression in the p300/cbp transfected cell and in control cells transfected with the fused gene promoter along with an “empty” plasmid. If reporter gene expression is higher or lower in the p300/cbp transfected cell, the gene is p300/cbp regulated.

2. Select a cell which expresses the gene of interest and transfect it with a vector expressing p300/cbp. Assay endogenous gene expression in the p300/cbp transfected cell and in control cells transfected with an “empty” plasmid. If gene expression is higher or lower in the p300/cbp transfected cell, the gene is p300/cbp regulated.

Note:

Preferably, verify that co-transfection did not induce a change in cellular microcompetition, a mutation in the gene promoter, or a change in methylation of gene promoter.

3. Transfect a cell with the gene promoter fused to a reporter gene, such as CAT or LUC. Contact the cell with an antibody against p300/cbp (or with a protein such as E1A). Assay gene expression in the antibody treated cell and in the untreated controls. If reporter gene expression is higher or lower in the antibody treated cell, the gene is p300/cbp regulated.

4. Select a cell which expresses a gene of interest. Contact the cell with an antibody against p300/cbp (or with a protein such as E1A). Assay gene expression in both the treated cell and in the untreated controls. If gene expression is higher or lower in the antibody treated cell, the gene is p300/cbp regulated.

5. Perform chromatin assembly of the gene promoter, for instance, with chromatin assembly extract from Drosophila embryos. Add a transcription factor during the chromatin assembly reactions. After the chromatin assembly reaction is complete add the p300/cbp proteins. Allow time for the interaction of the proteins with the chromatin template. Perform in vitro transcription reaction. Measure the concentration of the RNA products, by for instance, primer extension analysis. Compare to the RNA products before the addition of the p300/cbp proteins. If the addition of p300/cbp increased the concentration of the RNA products, the gene is p300/cbp regulated.

6. See more assays below.

### EXAMPLES

Direct evidence shows transactivation of certain promoters by p300/cbp (Manning 2001⁵⁹, Kraus 1999⁶⁰, Kraus 1998⁶¹).

Indirect evidence is available in studies with p300/cbp factors. Consider, for example, the p300/cbp factor GABP. GABP binds promoters and enhancers of many cellular genes including β₂ leukocyte integrin (CD18) (Rosmarin 1998⁶²), interleukin 16 (IL-16) (Bannert 1999, ibid), interleukin 2 (IL-2) (Avots 1997⁶³), interleukin 2 receptor β-chain (IL-2Rβ) (Lin 1993⁶⁴), IL-2 receptor γ-chain (IL-2 γc) (Markiewicz 1996⁶⁵), human secretory interleukin-1 receptor antagonist (secretory IL-lra) (Smith 1998⁶⁶), retinoblastoma (Rb) (Sowa 1997⁶⁷), human thrombopoietin (TPO) (Kamura 1997⁶⁸), aldose reductase (Wang 1993⁶⁹), neutrophil elastase (NE) (Nuchprayoon 1999⁷⁰, Nuchprayoon 1997⁷¹), folate binding protein (FBP) (Sadasivan 1994⁷²), cytochrome c oxidase subunit Vb (COXVb) (Basu 1993⁷³, Sucharov 1995⁷⁴), cytochrome c oxidase subunit IV (Carter 1994⁷⁵, Carter 1992⁷⁶), mitochondrial transcription factor A (mtTFA) (Virbasius 1994⁷⁷), β subunit of the FoFI ATP synthase (ATPsynβ) (Villena 1998⁷⁸), prolactin (prl) (Ouyang 1996⁷⁹) and the oxytocin receptor (OTR) (Hoare 1999⁸⁰) among others. For some of these genes, for instance, CD18, COXVb, COXIV, GABP binds to the promoter while for others, for example IL-2 and ATPsynβ, GABP binds an enhancer. More examples see below.

Another p300/cbp factor is NF-Y (see above). Mantovani 1998⁸¹ provides a list of genes which include a NF-Y binding site (Mantovani 1998, ibid, Table 1). For the listed genes, the table indicates whether the referenced studies report the presence of a proven binding site for a transcription factor close to the NF-Y binding site, whether cross-competition data with bonafide NF-Y binding sites are available, whether EMSA supershift experiments with anti NF-Y antibodies were performed, and whether the studies performed in vitro or in vivo transactivation studies with NF-Y. Some of the genes listed in the paper are MCH II, Ii, Mig, GP91 Phox, CD10, RAG-1, IL4, Thy-1, globin α, ζ, γ^{D} γ^{P}, Coll α2 (I) α1 (I), osteopontin, BSP, apoA-1, aldolase B, TAT, γ-GT, SDH, fibronectin, arg lyase, factor VIII, factor X, MSP, ALDH, LPL, ExoKII, FAS, TSP-1, FGF-4, α1-chim, Tr Hydr, NaKATPsea-3, PDFGβ, FerH, MHC IA2 B8, Cw2Ld and B7, MDR1, CYP1A1, c-JUN, Grp78, Hsp70, ADH2, GPAT, FPP, HMG, HSS, SREBP2, GHR, CP2, β-actin, TK, TopoIIα, I, III, III, IV, cdc25, cdc2, cyc1A, cyc1B1, E2F1, PLK, RRR2, H is H₂B, H is H3.

(5) p300/cbp Factor Kinase (p300/cbp Factor Phosphatase)

Definition

Assume F is a p300/cbp factor. If a molecule L stimulates phosphorylation or dephosphorylation of F, L is called “p300/cbp factor kinase” or “p300/cbp factor phosphatase,” respectively.

Exemplary Assays

1. Contact a system (for instance, organism, cell, cell lysate, chemical mixture) with a test molecule L. Use assays described in the section entitled “Assaying protein phosphorylation,” or similar assays, to uncover a change in phosphorylation of the p300/cbp factor of interest. An increase in phosphorylation indicates that L is a p300/cbp factor kinase, and a decrease indicates that L is a p300/cbp factor phosphatase.

EXAMPLE

Ras, Raf, MEK1, MEK 2, MEK4, ERK, JNK, three classes of ERK inactivators: type 1/2 serine/threonine phosphatases, such as PP2A, tyrosine-specific phosphatases (also called protein-tyrosine phosphatase, denoted PTP), such as PTP1B, and dual specificity phosphatases, such as MKP-1 which affect phosphorylation of a number of transcription factors, for instance, GABP, NF-κB. See also below.

(6) p300/cbp Agent

Definition

Assume the polynucleotide Pn binds the transcription complex C. Assume C contains p300/cbp. If a molecule L stimulates or suppresses binding of C to Pn, L is called “p300/cbp agent.” Specifically, such an agent can stimulate or suppress binding of p300/cbp to a p300/cbp factor, binding of p300/cbp to DNA, or binding of a p300/cbp factor to DNA.

Exemplary Assays

1. Contact a system (for instance, whole organism, cell, cell lysate, chemical mixture) with a test molecule L. Use assays described in the section entitled “Assaying binding to DNA,” or similar assays, to uncover a change in binding of the C to DNA. Specifically, assay for binding between p300/cbp and DNA, or p300/cbp and a p300/cbp factor, or p300/cbp factor and DNA.

### EXAMPLES

Examples of p300/cbp agents include sodium butyrate (SB), trichostatin A (TSA) trapoxin (for SB, TSA and trapoxin see in Espinos 1999⁸²), phorbol ester (phorbol 12-myristate 13-acetate, PMA, TPA), thapsigargin (for PMA and thapsigargin see Shiraishi 2000⁸³, for PMA see Herrera 1998⁸⁴, Stadheim 1998⁸⁵), retinoic acid (RA, vitamin A) (Yen 1999⁸⁶), interferon-γ (IFNγ) (Liu 1994⁸⁷, Nishiya 1997⁸⁸), heregulin (HRG, new differentiation factor, NDF, neuregulin, NRG) (Lessor 1998⁸⁹, Marte 1995⁹⁰, Sepp-Lorenzino 1996⁹¹, Fiddes 1998⁹²), zinc (Zn) (Park 1999⁹³, Kiss 1997⁹⁴), copper (Cu) (Wu 1999⁹⁵, Samet 1998⁹⁶, both studies also show phosphorylation of ERK1/2 by Zn), estron, estradiol (Migliaccio 1996⁹⁷, Ruzycky 1996⁹⁸, Nuedling 1999⁹⁹), interleukin 1β (IL-1β) (Laporte 1999¹⁰⁰, Larsen 1998¹⁰¹), interleukin 6 (IL-6) (Daeipour 1993¹⁰²), tumor necrosis factor α (TNFα) (Leonard 1999¹⁰³), transforming growth factor β (TGFβ) (Hartsough 1995¹⁰⁴, Yonekura 1999¹⁰⁵, oxytocin (OT) (Strakova 1998¹⁰⁶, Copland 1999¹⁰⁷, Hoare 1999, ibid). All studies show phosphorylation of ERK1/2 by these agents.

See more agents below.

Other examples include agents that modify oxidative stress, such as, diethyl maleate (DEM), a glutathione (GSH)-depleting agent, and N-acetylcysteine (NAC), an antioxidant and a precursor of GSH synthesis. See more agents below.

(7) Foreign p300/cbp Polynucleotide

Definition

Assume Pn is a polynucleotide foreign to organism R. If Pn is a p300/cbp polynucleotide, Pn is called “p300/cbp polynucleotide foreign to R.”

Exemplary Assays

Combine assays in the p300/cbp polynucleotide and foreign polynucleotide sections above.

### EXAMPLES

See examples in “p300/cbp virus” below.

(8) p300/cbp Virus

Definition

Assume Pn is a p300/cbp polynucleotide. If Pn is a segment of the genome of a virus V, V is called a “p300/cbp virus.”

Exemplary Assays

1. Verify that Pn is a p300/cbp polynucleotide (see assays above). Compare the sequence of Pn with the sequence of the published V genome. If the sequence is a segment of the V genome, Pn is a p300/cbp virus. If the V genome is not published, its sequence can be determined empirically.

2. Verify that Pn is a p300/cbp polynucleotide (see assays above) by hybridizing Pn to the V genome. If Pn hybridizes, Pn is a p300/cbp virus.

### EXAMPLES

Direct evidence shows transactivation of certain viruses by p300/cbp. See, for instance, Subramanian 2002¹⁰⁸ on Epstein-Barr virus, Banas 2001¹⁰⁹, Deng 2000¹¹⁰ on HIV-1, Cho 2001¹¹¹ on SV40 and polyomavirus, Wong 1994¹¹², on adenovirus type 5. See also Hottiger 2000 (ibid), a review on viral replication and p300/cbp.

Indirect evidence is available in studies with p300/cbp factors. Consider, for instance, the p300/cbp factor GABP. Since GABP binds p300/cbp (see above), a complex on DNA which includes GABP, also includes p300/cbp. The DNA motif (A/C)GGA(A/T)(G/A), termed the N-box, is the core binding sequence for GABP. The N-box is the core binding sequence of many viral enhancers including the polyomavirus enhancer area 3 (PEA3) (Asano 1990¹¹³), adenovirus E1A enhancer (Higashino 1993¹¹⁴), Rous Sarcoma Virus (RSV) enhancer (Laimins 1984¹¹⁵), Herpes Simplex Virus 1 (HSV-1) (in the promoter of the immediate early gene ICP4) (LaMarco 1989¹¹⁶, Douville 1995¹¹⁷), Cytomegalovirus (CMV) (IE-1 enhancer/promoter region) (Boshart 1985¹¹⁸), Moloney Murine Leukemia Virus (Mo-MuLV) enhancer (Gunther 1994¹¹⁹), Human Immunodeficiency Virus (HIV) (the two NF-κB binding motifs in the HIV LTR) (Flory 1996¹²⁰), Epstein-Barr virus (EBV) (20 copies of the N-box in the +7421/+8042 oriP/enhancer) (Rawlins 1985¹²¹) and Human T-cell lymphotropic virus (HTLV) (8 N-boxes in the enhancer (Mauclere 1995¹²²) and one N-box in the LTR (Komfeld 1987¹²³)). Moreover, some viral enhancers, for example SV40, lack a precise N-box, but still bind the GABP transcription factor (Bannert 1999, ibid).

Ample evidence exists which supports the binding of GABP to the N-boxes in these viral enhancers. For instance, Flory, et al., (1996, ibid) show binding of GABP to the HIV LTR, Douville, et al., (1995, ibid) show binding of GABP to the promoter of ICP4 of HSV-1, Bruder, et al., 1991¹²⁴ and Bruder, et al., 1989¹²⁵ show binding of GABP to the adenovirus E1A enhancer element I, Ostapchuk, et al., 1986¹²⁶ show binding of GABP (called EF-1A in this paper) to the polyomavirus enhancer and Gunther, et al., (1994, ibid) show binding of GABP to Mo-MuLV.

Other studies demonstrate competition between these viral enhancers and enhancers of other viruses. Scholer and Gruss, 1984¹²⁷ show competition between the Moloney Sarcoma Virus (MSV) enhancer and SV40 enhancer and also competition between the RSV enhancer and the BK virus enhancer.

Another p300/cbp factor is NF-Y (see above). Mantovani 1998 (ibid) provides a list of viruses which include a NF-Y binding site (Table 1). The list includes HBV S, MSV LTR, RSV LTR, ad EIIL II, Ad MK, CMV gpUL4, HSV IE IOk, VZV 0RF62, MVM P4.

More Exemplary Assays for Identification of a Polynucleotide Pn as a p300/cbp Polynucleotide:

1. Take a cell of interest. Modify the copy number of Pn in the cell (by, for instance, transfection, infection, mutation, etc, see also above). Assay binding of all p300/cbp factors to Pn. If a p300/cbp factor binds Pn, Pn is a p300/cbp polynucleotide.

2. Assay binding of a p300/cbp factor to endogenous DNA or to exogenous DNA following introduction to the cell of interest. Modify the copy number of Pn in the cell. Assay binding of the p300/cbp factor again. If binding changed, Pn is a p300/cbp polynucleotide.

3. Identify a binding site on Pn for p300/cbp or a p300/cbp factor by computerized sequence analysis.

4. Take a cell of interest. Transfect the cell with a vector that expresses a reporter gene under the control of a promoter of a p300/cbp regulated gene. Modify the copy number of Pn in the cell (by, for instance, transfection, infection, mutation, etc, see also above). Assay expression of the reporter gene and compare to cells with unmodified copy number of Pn. If expression in the Pn modified cell is different than controls, Pn is a p300/cbp polynucleotide.

5. Take a cell of interest that expresses an endogenous p300/cbp regulated gene. Modify the copy number of Pn in the cell (by, for instance, transfection, infection, mutation, etc, see also above). Assay expression of the p300/cbp regulated gene and compare to cells with an unmodified copy number of Pn (for instance, in cells transfected with an empty plasmid). If expression in the Pn transfected cell is different than controls, Pn is a p300/cbp polynucleotide.

6. Take a cell of interest. Infect the cell with a p300/cbp virus. Modify the copy number of Pn in the cell (by, for instance, transfection, infection, mutation, etc, see also above). Assay viral replication and compare to cells with unmodified copy number of Pn (for instance, in cells infected with a non p300/cbp virus). If viral replication is different, Pn is a p300/cbp polynucleotide.

7. Compare the sequence of Pn to the genome of a p300/cbp virus using a sequence alignment algorithm such as BLAST. If a segment of the Pn sequence is identical (or homologous) to a segment in viral genome, Pn is a p300/cbp polynucleotide. A polynucleotide of at least 18 nucleotides should be sufficient to ensure specificity and validate alignment.

8. Try to hybridize Pn to the genome of a p300/cbp virus. If Pn hybridizes to the viral genome, Pn is a p300/cbp polynucleotide. Hybridization conditions should be sufficiently stringent to permit specific, but not promiscuous, hybridization. Such conditions are well known in the art.

c) GABP Related Elements

(1) GABP

Definition

A member of the GA binding protein (GABP) family of proteins is called GABP.

Notes:

1. GA binding protein (GABP) is also called Nuclear Respiratory Factor 2 (NRF-2)¹²⁸, E4 Transcription factor 1 (E4TF1)¹²⁹, and Enhancer Factor 1A (EF-1A)¹³⁰.

2. The literature lists five subunits of GABP: GABPα, GABPβ1, GABPβ2 (together called GABPβ), GABPγ1 and GABPγ2 (together called GABPγ). GABPα is an ets-related DNA-binding protein which binds the DNA motif (A/C)GGA(A/T)(G/A), termed the N-box. GABPα forms a heterocomplex with GABPβ which stimulates transcription efficiently both in vitro and in vivo. GABPα also forms a heterocomplex with GABPγ, but the heterodimer does not stimulate transcription. The degree of transactivation by GABP appears to correlate with the relative intracellular concentrations of GABPβ and GABPγ. An increase in GABPβ relative to GABPγ increases transcription, while an increase of GABPγ relative to GABPβ decreases transcription. The degree of transactivation by GABP is, therefore, a function of the ratio between GABPβ and GABPγ. Control of this ratio within the cell regulates transcription of genes with binding sites for GABP (Suzuki 1998¹³¹).

3. See sequences of GABP genes and GABP proteins in the List of Sequences below.

Exemplary Assays

1. GABP may be identified using antibodies in binding assays, oligonucleotide probes in hybridization assays, etc. (see protocols for binding and hybridization assays below).

### EXAMPLES

See examples below.

(2) GABP Polynucleotide

Definition

Assume the polynucleotide Pn binds the transcription complex C. If C contains GABP, Pn is called a “GABP polynucleotide.”

Exemplary Assays

1. Take a cell of interest. Modify the copy number of Pn (by, for instance, transfection, infection, mutation, etc, see also above). Use assays described in the section entitled “Detailed description of standard elements,” or similar assays, to test if the protein-Pn complexes contain GABP.

2. See more assays below.

### EXAMPLES

See below in GABP virus and GABP regulated gene.

(3) GABP Regulated (Gene, Polypeptide)

Definition

Assume the gene G is transactivated, or suppressed by the transcription complex C. If C contains GABP, the gene G, and the polypeptide encoded by G, are called “GABP regulated.”

Exemplary Assays

1. Co-transfect a cell with the gene promoter of interest fused to a reporter gene, such as CAT or LUC, and a vector expressing GABP. Assay reporter gene expression in the GABP transfected cell and in control cells transfected with the fused gene promoter along with an “empty” plasmid, that is, with a plasmid identical to the plasmid expressing GABP but without the GABP coding region. If the reporter gene expression is higher or lower in the GABP transfected cell compared to the “empty” plasmid transfected cell, the gene is GABP regulated.

2. Select a cell which endogenously expresses the gene of interest and transfect it with a vector expressing GABP. Assay the gene expression in the GABP transfected cell and in control cells transfected with an “empty” plasmid (see above). If gene expression is higher or lower in the GABP transfected cell compared to the “empty” plasmid transfected cell, the gene is GABP regulated.

Note:

Preferably, verify that co-transfection did not induce a change in cellular microcompetition, a mutation in the gene promoter, or a change in methylation of the gene promoter.

3. Transfect a cell with the gene promoter of interest fused to a reporter gene, such as CAT or LUC. Contact the cell with an antibody against GABP. Assay gene expression in the antibody treated cell and untreated controls. If the reporter gene expression is higher or lower in the antibody treated cell compared to the untreated controls, the gene is GABP regulated.

4. Select a cell which expresses a gene of interest. Contact the cell with an antibody against GABP. Assay gene expression in both the treated cell and untreated controls. If gene expression is higher or lower in the antibody treated cell compared to the untreated controls, the gene is GABP regulated.

5. See more assays below.

### EXAMPLES

GABP binds promoters and enhancers of many cellular genes including (see above). More examples see below.

(4) GABP Kinase (GABP Phosphatase)

Definition

If a molecule L stimulates phosphorylation or dephosphorylation of GABP, L is called “GABP kinase” or “GABP phosphatase,” respectively.

Exemplary Assays

1. Contact a system (for instance, organism, cell, cell lysate, chemical mixture) with a test molecule L. Use assays described in the section entitled “Detailed description of standard elements,” or similar assays, to uncover a change in phosphorylation of GABP. An increase in phosphorylation indicates that L is a GABP kinase, a decrease indicates that L is a GABP phosphatase.

EXAMPLE

Ras, Raf, MEK1, MEK 2, MEK4, ERK, JNK, three classes of ERK inactivators: type 1/2 serine/threonine phosphatases, such as PP2A, tyrosine-specific phosphatases (also called protein-tyrosine phosphatase, denoted PTP), such as PTP1B, and dual specificity phosphatases, such as MKP-1 which affect phosphorylation GABP. See also below.

(5) GABP Agent

Definition

Assume the polynucleotide Pn binds the transcription complex C. Assume C contains GABP. If a molecule L stimulates or suppresses binding of C to Pn, L is called “GABP agent.” Specifically, such agent can stimulate or suppress binding of GABP family members to each other, binding of GABP to other transcription factors, or binding of GABP to DNA.

Exemplary Assays

1. Contact a system (for instance, whole organism, cell, cell lysate, chemical mixture) with a test molecule L. Use assays described in the section entitled “Detailed description of standard elements,” or similar assays, to uncover a change in binding of the complex C to DNA. Specifically, assay binding of GABP family members to each other, binding of GABP to other transcription factors, or binding of GABP to DNA.

### EXAMPLES

Examples of GABP agents include sodium butyrate (SB), trichostatin A (TSA), trapoxin (for SB, TSA and trapoxin see in Espinos 1999, ibid), phorbol ester (phorbol 12-myristate 13-acetate, PMA, TPA), thapsigargin (for PMA and thapsigargin see Shiraishi 2000, ibid, for PMA see Herrera 1998, ibid, Stadheim 1998, ibid), retinoic acid (RA, vitamin A) (Yen 1999, ibid), interferon-γ (IFNγ) (Liu 1994, ibid, Nishiya 1997, ibid), heregulin (HRG, new differentiation factor, NDF, neuregulin, NRG) (Lessor 1998, ibid, Marte 1995, ibid, Sepp-Lorenzino 1996, ibid, Fiddes 1998, ibid), zinc (Zn) (Park 1999, ibid Kiss 1997, ibid), copper (Cu) (Wu 1999, ibid, Samet 1998, ibid, both studies also show phosphorylation of ERK1/2 by Zn), estron, estradiol (Migliaccio 1996, ibid, Ruzycky 1996, ibid, Nuedling 1999, ibid), interleukin 1β (IL-1β) (Laporte 1999, ibid, Larsen 1998, ibid), interleukin 6 (IL-6) (Daeipour 1993, ibid), tumor necrosis factor α (TNFα) (Leonard 1999, ibid), transforming growth factor β (TGFβ) (Hartsough 1995, ibid, Yonekura 1999, ibid, oxytocin (OT) (Strakova 1998, ibid, Copland 1999, ibid, Hoare 1999, ibid). All studies show phosphorylation of ERK1/2 by these agents. See more agents below.

Other examples include agents which modify oxidative stress, such as, diethyl maleate (DEM), a glutathione (GSH)-depleting agent, and N-acetylcysteine (NAC), an antioxidant and a precursor of GSH synthesis. See details and more agents below.

(6) Foreign GABP Polynucleotide

Definition

Assume Pn is a polynucleotide foreign to organism R. If Pn is a GABP polynucleotide, Pn is called “GABP polynucleotide foreign to R.”

Exemplary Assays

Combine assays in the GABP polynucleotide and foreign polynucleotide sections above.

### EXAMPLES

See examples in “GABP virus” below.

(7) GABP Virus

Definition

Assume Pn is a GABP polynucleotide. If Pn is a segment of the genome of a virus V, V is called a “GABP virus.”

Exemplary Assays

1. Verify that Pn is a GABP polynucleotide (see assays above). Compare the sequence of Pn with the sequence of the published V genome. If the sequence is a segment of the V genome, Pn is a GABP virus. If the V genome is not published, determine the sequence empirically and compare.

2. Verify that Pn is a GABP polynucleotide (see assays above) by hybridizing Pn to the V genome (see exemplary hybridization assays in the section entitled “Detailed description of standard elements”). If Pn hybridizes, Pn is a GABP virus.

3. Verify that Pn is a GABP polynucleotide by identifying in Pn the DNA motif (A/C)GGA(A/T)(G/A), termed the N-box. Preferably, identify two N-boxes separated by multiples of 0.5 helical turns (HT), up to 3.0 HT (there are 10 base pair per HT) in Pn (see more details below).

### EXAMPLES

See above. See also below.

More Exemplary Assays for Identification of a Polynucleotide Pn as a GABP Polynucleotide:

1. Take a cell of interest. Assay binding of GABP to endogenous Pn, or exogenous Pn following introduction of Pn to the cell of interest. If a GABP binds Pn, Pn is a GABP polynucleotide.

2. Identify a polynucleotide Pn1 that binds GABP. Assay binding of a GABP to endogenous Pn1, or exogenous Pn1 following introduction of Pn1 to a cell of interest.

Modify the copy number of a second polynucleotide, Pn2, in the cell. Assay binding of GABP to Pn1 again. If binding to Pn1 changed, Pn2 is a GABP polynucleotide.

3. Identify a binding site on Pn for GABP by computerized sequence analysis.

4. Take a cell of interest. Transfect the cell with a vector that expresses a reporter gene under the control of a promoter of a GABP regulated gene. Modify the copy number of Pn in the cell (by, for instance, transfection, infection, mutation, etc, see also above). Assay expression of the reporter gene and compare to cells with unmodified copy number of Pn. If expression in the Pn modified cell is different than controls, Pn is a GABP polynucleotide.

5. Take a cell of interest which expresses an endogenous GABP regulated gene. Modify the copy number of Pn in the cell (by, for instance, transfection, infection, mutation, etc, see also above). Assay expression of the GABP regulated gene and compare to cells with unmodified copy number of Pn. If expression in the Pn modified cell is different than controls, Pn is a GABP polynucleotide.

6. Take a cell of interest. Infect the cell with a GABP virus. Modify the copy number of Pn in the cell (by, for instance, transfection, infection, mutation, etc, see also above). Assay viral replication and compare to cells with unmodified copy number of Pn (for instance, in cells infected with a non GABP virus). If viral replication is different, Pn is a GABP polynucleotide.

7. Compare the sequence of Pn to the genome of a GABP virus using a sequence alignment algorithm such as BLAST. If a segment of the Pn sequence is identical (or homologous) to a segment in viral genome, Pn is a GABP polynucleotide. A polynucleotide of at least 18 nucleotides should be sufficient to ensure specificity and validate alignment.

8. Try to hybridize Pn to the genome of a GABP virus. If Pn hybridizes to the viral genome, Pn is a GABP polynucleotide. Hybridization conditions should be sufficiently stringent to permit specific, but not promiscuous hybridization. Such conditions are well known in the art.

d) Agents Related Elements

(1) Modulator

Definition

Consider a polynucleotide Pn. An agent, or treatment (called agent for short), is called “modulator” if the agent modifies microcompetition with Pn, modifies at least one effect of microcompetition with Pn, or modifies at least one effect of another foreign polynucleotide-type disruption.

Notes:

1. A treatment, such as irradiation, can also be a modulator. In principle, according to the definition, any foreign polynucleotide-type disruption is a modulator.

Exemplary Assays

1. Assay the effect of an agent on Pn copy number.

Specifically, take a biological system (e.g. cell, whole organism, etc). Modify the copy number of Pn (by, for instance, transfection, infection, mutation, etc, see above). Call this cell the Pn cell. Assay the Pn copy number in the Pn cell (see above). Contact the biological system with an agent of interest. Assay again the Pn copy number. If the Pn copy number is higher or lower compared to the copy number in Pn cells not contacted with the agent, the agent is a modulator.

2. Assay the effect of an agent on binding of p300/cbp to Pn, directly or in a complex.

Specifically, take a biological system (e.g. cell, whole organism, etc). Modify the copy number of Pn (by, for instance, transfection, infection, mutation, etc, see above). Call this cell the Pn cell. Assay binding of p300/cbp to Pn (see above). Contact the biological system with an agent of interest. Assay again the binding of p300/cbp to Pn. If the binding is higher or lower compared to binding in Pn cells not contacted with the agent, the agent is a modulator.

3. Assay the effect of an agent on binding of GABP to Pn.

Specifically, take a biological system (e.g. cell, whole organism, etc). Modify the copy number of Pn (by, for instance, transfection, infection, mutation, etc, see above). Call this cell the Pn cell. Assay binding of GABP to Pn (see above). Contact the biological system with an agent of interest. Assay again the binding of GABP to Pn. If binding is higher or lower compared to binding in Pn cells not contacted with the agent, the agent is a modulator.

4. Assay the effect of an agent on binding of p300/cbp to GABP.

Specifically, take a biological system (e.g. cell, whole organism, etc). Modify the copy number of Pn (by, for instance, transfection, infection, mutation, etc, see above). Call this cell the Pn cell. Assay binding of p300/cbp to GABP (see above). Contact the biological system with an agent of interest. Assay again the binding of p300/cbp to GABP. If binding is higher or lower compared to binding in Pn cells not contacted with the agent, the agent is a modulator.

5. Assay the effect of an agent on expression of a disrupted gene and/or polypeptide.

Specifically, take a biological system (e.g. cell, whole organism, etc). Modify the copy number of Pn (by, for instance, transfection, infection, mutation, etc, see above). Call this cell the Pn cell. Identify a disrupted gene and/or polypeptide (see assays above). Contact the biological system with an agent of interest. Assay the bioactivity of the disrupted gene and/or polypeptide. If the bioactivity of the disrupted gene and/or polypeptide is higher or lower compared to the bioactivity in Pn cells not contacted with the agent, the agent is a modulator.

### EXAMPLES

See below in constructive/disruptive.

(2) Constructive/Disruptive

Definition

A modulator, which attenuates or accentuates microcompetition with a foreign polynucleotide, attenuates or accentuates at least one effect of microcompetition with a foreign polynucleotide, or attenuates or accentuates at least one effect of another foreign polynucleotide-type disruption, is called “constructive” or “disruptive,” respectively.

Notes:

1. A modulator can be both constructive and disruptive.

2. Consider a gene suppressed by microcompetition with a foreign polynucleotide. Consider such a gene in a cell without a foreign polynucleotide. Now consider a mutation which reduces the gene bioactivity. An agent which stimulates expression of such mutated gene will also be called constructive. If, on the other hand, the mutation stimulates the gene bioactivity, an agent which suppresses its bioactivity will also be called constructive.

3. A constructive agent can be an agonist, if it stimulates expression of a gene suppressed by microcompetition with a foreign polynucleotide, or if is stimulates bioactivity of a polypeptide encoded by such a gene. A constructive agent can also be an antagonist if it inhibits expression of a gene stimulated by microcompetition with a foreign polynucleotide, or inhibits the bioactivity of a polypeptide encoded by such a gene.

4. A foreign polynucleotide-type disruption can be constructive.

Exemplary Assays

1. See assays in Modulator section above. In these assay if either;

(a) Pn copy number in the Pn cell contacted with the agent is higher relative to Pn cells not contacted by the agent;

(b) binding of p300/cbp to Pn in the Pn cell contacted with the agent is higher compared to binding in Pn cells not contacted with the agent;

(c) binding of GABP to Pn in the Pn cell contacted with the agent is higher compared to binding in Pn cells not contacted with the agent;

(d) binding of p300/cbp to GABP in the Pn cell contacted with the agent is higher or lower compared to binding in Pn cells not contacted with the agent;

(e) bioactivity of the disrupted gene and/or polypeptide in the Pn cell contacted with the agent is higher (for genes and/or polypeptides with suppressed bioactivity) compared to the bioactivity in Pn cells not contacted with the agent;

the agent is constructive.

If the effect is in the opposite direction, the agent is disruptive.

### EXAMPLES

Antiviral drugs, sodium butyrate, garlic, etc. See more examples in Treatment section below.

2. Detailed description of standard elements

a) General comments

The elements of the present invention may include, as their own elements, standard methods in molecular biology, microbiology, cell biology, transgenic biology, recombinant DNA, immunology, cell culture, pharmacology, and toxicology, well known in the art. The following sections provide details for some standard methods. Complete descriptions are available in the literature. For instance, see the “Current Protocols” series published by John Wiley & Sons. The following list provides a sample of books in the series: Current Protocols in Cell Biology, edited by: Juan S. Bonifacino, Mary Dasso, Jennifer Lippincott-Schwartz, Joe B Harford, and Kenneth M Yamada; Current Protocols in Human Genetics, edited by: Nicholas C Dracopoli, Jonathan L Haines, Bruce R Korf, Cynthia C Morton, Christine E Seidman, JG Seidman, Douglas R Smith; Current Protocols in Immunology, edited by: John E Coligan, Ada M Kruisbeek, David H Margulies, Ethan M Shevach, and Warren Strober; Current Protocols in Molecular Biology, edited by: Frederick M Ausubel, Roger Brent, Robert E Kingston, David D Moore, J G Seidman, John A Smith, and Kevin Struhl; Current Protocols in Nucleic Acid Chemistry, edited by: Serge L Beaucage, Donald E Bergstrom, Gary D Glick, Roger A Jones; Current Protocols in Pharmacology, edited by: SJ Enna, Michael Williams, John W Ferkany, Terry Kenakin, Roger D Porsolt, James P Sullivan; Current Protocols in Protein Science, edited by: John E Coligan, Ben M Dunn, Hidde L Ploegh, David W Speicher, Paul T Wingfield; Current Protocols in Toxicology, edited by: Mahin Maines (Editor-in-Chief), Lucio G Costa, Donald J Reed, Shigeru Sassa, I Glenn Sipes. The following lists includes more books with standard methods. Basic DNA and RNA Protocols (Methods in Molecular Biology, Vol 58), edited by Adrian J Harwood, Humana Press, 1994; DNA-Protein Interactions: Principles and Protocols (Methods in Molecular Biology, Volume 148), edited by Tom Moss, Humana Press, 2001; Transcription Factor Protocols (Methods in Molecular Biology), edited by Martin J Tymms, Humana Press, 2000; Gene Transcription: A Practical Approach, edited by B D Hames, and S J Higgins, IRL Press at Oxford University Press, 1993; Gene Transcription, DNA Binding Proteins: Essential Techniques, edited by Kevin Docherty, Jossey Bass, 1997; Gene Probes Principles and Protocols (Methods in Molecular Biology, 179), edited by Marilena Aquino de Muro and Ralph Rapley, Humana Press, 2001; Gene Isolation and Mapping Protocols (Methods in Molecular Biology Vol 68), edited by Jackie Boultwood and Jacqueline Boultwood, Humana Press, 1997; Gene Targeting Protocols (Methods in Molecular Biology, Vol 133), edited by Eric B Kmiec and Dieter C Gruenert, Humana Press 2000; Epitope Mapping Protocols (Methods in Molecular Biology, Vol 66), edited by Glenn E Morris, Humana Press, 1996; Protein Targeting Protocols (Methods in Molecular Biology, Vol 88), edited by Roger A Clegg, Humana Press, 1998; Monoclonal Antibody Protocols (Methods in Molecular Biology, 45), edited by William C Davis, Humana Press, 1995; Immunochemical Protocols (Methods in Molecular Biology Vol 80), edited by John D Pound, Humana Press, 1998; Immunoassay Methods and Protocols (Methods in Molecular Biology), edited by Andrey L Ghindilis, Andrey R Pavlov and Plamen B Atanassov, Humana Press, 2002; In situ Hybridization Protocols (Methods in Molecular Biology, 123), edited by Ian A Darby, Humana Presse, 2000; Bioluminescence Methods & Protocols, edited by Robert A Larossa, Humana Press, 1998; Affinity Chromatography: Methods and Protocols (Methods in Molecular Biology), etided by Pascal Bailon, George K Ehrlich, Wen-Jian Fung, wo Berthold and Wolfgang Berthold, Humana Press, 2000; Protocols for Oligonucleotide Conjugates: Synthesis and Analytical Techniques (Methods in Molecular Biology, Vol 26), edited by Sudhir Agrawal, Humana Press, 1993; RNA Isolation and Characterization Protocols (Methods in Molecular Biology, No 86), edited by Ralph Rapley and David L Manning, Humana Press, 1998; Protocols for Oligonucleotides and Analogs: Synthesis and Properties (Methods in Molecular Biology, 20), edited by Sudhir Agrawal, Humana Press, 1993; Basic Cell Culture Protocols (Methods in Molecular Biology, 75), edited by Jeffrey W Pollard and John M Walker, Humana Press, 1997; Quantitative PCR Protocols (Methods in Molecular Medicine, 26), edited by Bemd Kochanowski and Udo Reischl, Humana Press, 1999; In situ PCR Techniques, edited by Omar Bagasra and John Hansen, John Wiley & Sons, 1997; PCR Cloning Protocols: From Molecular Cloning to Genetic Engineering (Methods in Molecular Biology, No 67), edited by Bruce A White, Humana Press, 1996; PRINS and In situ PCR Protocols (Methods in Molecular Biology, 71), edited by John R Gosden, Humana Press, 1996; PCR Protocols: Current Methods and Applications (Methods in Molecular Biology, 15), edited by Bruce A White, Humana Press 1993; Transmembrane Signaling Protocols (Methods in Molecular Biology, Vol 84), edited by Dafna Bar-Sagi, Humana Press, 1998; Chemokine Protocols (Methods in Molecular Biology, 138), edited by Amanda E I Proudfoot, Timothy N C Wells and Chris Power, Humana Press, 2000; Baculovirus Expression Protocols (Methods in Molecular Biology, Vol 39), edited by Christopher D Richardson, Humana Press, 1998; Recombinant Gene Expression Protocols (Methods in Molecular Biology, 62), edited by Rocky S Tuan, Humana Press, 1997; Recombinant Protein Protocols: Detection and Isolation (Methods in Molecular Biology, Vol 63), edited by Rocky S Tuan, Humana Press, 1997; DNA Repair Protocols: Eukaryotic Systems (Methods in Molecular Biology, Vol 113), edited by Daryl S Henderson, Humana Press, 1999; DNA Sequencing Protocols, editors Hugh G Griffin and Annette M Griffin, Humana Press, 1993; Protein Sequencing Protocols (Methods in Molecular Biology, No 64), edited by Bryan John Smith, Humana Press, 2001; Gene Transfer and Expression Protocols (Methods in Molecular Biology, Vol 7), edited by E J Murray, Humana Press, 1991; Transgenesis Techniques, Principles and Protocols (Methods in Molecular Biology, 180), edited by Alan R Clarke, Humana Press, 2002; Regulatory Protein Modification Techniques and Protocols (Neuromethods, 30), edited by Hugh C Hemmings, Humana Press, 1996; Downstream Processing of Proteins Methods and Protocols (Methods in Biotechnology, 9), edited by Mohamed A Desai, Humana Press, 2000; DNA Vaccines Methods and Protocols (Methods in Molecular Medicine, 29), edited by Douglas B Lowrie and Robert Whalen, Humana Press, 1999; DNA Arrays Methods and Protocols (Methods in Molecular Biology, 170), edited by Jang B Rampal, Humana Press, 2001; Drug-DNA Interaction Protocols, editor Keith Fox, Humana Press, 1997; In vitro Mutagenesis Protocols, edited Michael K. Trower, Humana Press, 1996; In vitro Toxicity Testing Protocols (Methods in Molecular Medicine, 43), edited by Sheila O'Hare and C K Atterwill, Humana Press, 1995; Mutation Detection: A Practical Approach (Practical Approach Series (Paper), No 188), edited by Richard G H Cotton, E Edkins and S Forrect, Irl Press, 1998; Herpes Simplex Virus Protocols (Methods in Molecular Medicine, 10), edited by S Moira Brown and Alasdair R MacLean, Humana Press, 1997; HIV Protocols (Methods in Molecular Medicine, 17), edited by Nelson Michael and Jerome H Kim, Humana Press, 1999; Cytomegalovirus Protocols (Methods in Molecular Medicine, 33), edited by John Sinclair, Humana Press, 1999; Antiviral Methods and Protocols (Methods in Molecular Medicine, 24), edited by Derek Kinchington and Raymond F Schinazi, Humana Press, 1999; Epstein-Barr Virus Protocols (Methods in Molecular Biology Vol 174), edited by Joanna B Wilson and Gerhard H W May, Humana Press, 2001; Adenovirus Methods and Protocols (Methods in Molecular Medicine, Vol 21), edited by William S M Wold, Humana Press, 1999; Molecular Methods for Virus Detection, edited by Danny L Wiedbrauk and Daniel H Farkas, Academic Press, 1995; Diagnostic Virology Protocols (Methods in Molecular Medicine, No 12), edited by John R Stephenson and Alan Warnes, Humana Press, 1998. A more extensive list of books with detailed description of standard methods is available at the Promega web site: http:www.promega.com/catalog/category.asp?catalog%5Fname=Promega %5FProducts &category %5Fname=Books&description %5Ftext=Books&Page=1. The Promega list includes 260 books.

For each element, one or more exemplary protocols are presented. All examples included in the application should be considered as illustrations, and, therefore, should not be construed as limiting the invention in any way.

More details regarding the presented exemplary protocols, and details of other protocols that can be used instead of the presented protocols, are available in the cited references, and in the books listed above. The contents of all references cited in the application, including, but not limited to, abstracts, papers, books, published patent applications, issued patents, available in paper format or electronically, are hereby expressly and entirely incorporated by reference.

The following sections first present protocols for formulation of a drug candidate, then protocols, that as elements of above assays, can be used to test a drug candidate for a desired biological activity during drug discovery, development and clinical trials. The assays can also be used for diagnostic purposes. Finally, the following sections also present protocols for effective use of a drug as treatment.

b) Formulation Protocols

One aspect of the invention pertains to administration of a molecule of interest, equivalent molecules, or homologous molecules, isolated from, or substantially free of contaminating molecules, as treatment of a chronic disease.

(1) Definitions

(a) Molecule of Interest

The terms “molecule of interest” or “agent,” is understood to include small molecules, polypeptides, polynucleotides and antibodies, in a form of a pharmaceutical or nutraceutical.

(b) Equivalent Molecules

The term “equivalent molecules” is understood to include molecules having the same or similar activity as the molecule of interest, including, but not limited to, biological activity, chemical activity, pharmacological activity, and therapeutic activity, in vitro or in vivo.

(c) Homologous Molecules

The term “homologous molecules” is understood to include molecules with the same or similar chemical structure as the molecule of interest.

In one exemplary embodiment, homologous molecules may be synthesized by chemical modification of a molecule of interest, for instance, by adding any of a number of chemical groups, including but not limited to, sugars (i.e. glycosylation), phosphates, acetyls, methyls, and lipids. Such derivatives may be derived by the covalent linkage of these or other groups to sites within a molecule of interest, or in the case of polypeptides, to the N-, or C-termini, or polynucleotides, to the 5′ or 3′ ends.

In one exemplary embodiment, homologous polypeptides or homologous polynucleotides include polypeptides or polynucleotides that differ by one or more amino acid, or nucleotides, respectively, from the polypeptide or polynucleotide of interest. The differences may arise from substitutions, deletions or insertions into the initial sequence, naturally occurring or artificially formulated, in vivo or in vitro. Techniques well known in the art may be applied to introduce mutations, such as point mutations, insertions or deletion, or introduction of premature translational stops, leading to the synthesis of truncated polypeptides. In every case, homologs may show attenuated activities compared to the original molecules, exaggerated activities, or may express a subset or superset of the total activities elicited by the original molecule. In these ways, homologs of constructive or disruptive polypeptides or polynucleotides have biological activities either diminished or expanded compared to the original molecule. In every case, a homolog may, or may not prove more effective in achieving a desired therapeutic effect. Methods for identifying homologous polypeptides or polynucleotides are well known in the art, for instance, molecular hybridization techniques, including, but not limited to, Northern and Southern blot analysis, performed under variable conditions of temperature and salt, can formulate nucleic acid sequences with different levels of stringency. Suitable protocols for identifying homologous polypeptides or polynucleotides are well known in the art (see, for instance, Sambrook 2001¹³² and above listed books of standard protocols). Homologous polypeptides or polynucleotides can also be generated, for instance by a suitable combinatorial approach.

It is well known in the art that the ribonucleotide triplets, termed codons, encoding each amino acid, comprise a set of similar sequences typically differing in their third position. Variations, known as degeneracy, occur naturally, and in practice mean that any given amino acid may be encoded by more than one codon. For instance, the amino acids arginine, serine and leucine can be encoded by 6 codons. As a result, in one exemplary embodiment, homologous DNA and RNA polynucleotides can be produced which encode the same polypeptide of interest.

In another exemplary embodiment, a set of homologous polypeptides may be generated by incorporating a population of synthetic oligodeoxyribonucleotides into expression vectors already carrying additional portions of the polypeptide of interest. The site into which the oligonucleotide-gene fusion is incorporated must include appropriate transcriptional and translational regulatory sequences flanking the inserted oligonucleotides to permit expression in host cells. Once introduced into an appropriate host cell, the resulting collection of gene-oligonucleotide recombinant vectors expresses polypeptide variants of the polypeptide of interest. The expressed polypeptide may be separately purified by cloning the vector bearing host cells, or by employing appropriate bacteriophage vectors, such as gt-11 or its derivatives, and screening plaques with antibodies against the polypeptide of interest, or against an immunological tag included in the recombinants.

(d) Isolated

The terms “isolated from, or substantially free of contaminating molecules” is understood to include a molecule containing less than about 20% contaminating molecules, based on dry weight calculations, preferably, less than about 5% contaminating molecules.

The terms “isolated” or “purified” do not refer to materials in a natural state, or materials separated into elements without further purification. For example, separating a preparation of nucleic acids by gel electrophoresis, by itself, does not constitute purification unless the individual molecular species are subsequently isolated from the gel matrix.

In one exemplary embodiment, a polynucleotide encoding a polypeptide of interest is ligated into a fusion polynucleotide encoding another polypeptide which facilitates purification, for instance, a polypeptide with readily available antibodies, such as VP6 rotavirus capsid protein, a vaccinia virus capsid protein, or the bacterial GST protein. When expressed, the facilitator polypeptide enables purification of the polypeptide of interest and immunological identification of host cells which express it. In the case of GST-fusion proteins, purification may be achieved by use of glutathione-conjugated sepharose beads in affinity chromatographic techniques well known in the art (see, for instance, Ausubel 1998¹³³).

In a related exemplary embodiment, the fusion polypeptide includes a polyamino acid tract, such as the polyhistidine/enterokinase cleavage site, which confers physical properties that inherently enable purification. In this example, purification may be achieved through nickel metal affinity chromatography. Once purified, the polyhistidine tract included to enable purification can be removed by treatment with enterokinase in vitro to release the polypeptide fragment of interest.

For molecules synthesized by an organism, for instance, polypeptides or polynucleotides synthesized by human subjects, in a preferred exemplary embodiment, a purified polynucleotide or polypeptide is free of other molecules synthesized by same organism, accomplished, for example, by expression of a human gene in a non-human host cell.

The following sections present standard protocols for the formulation of certain types of agents.

(2) Small Molecules

One aspect of the invention pertains to administration of a small molecule of interest, equivalent small molecules, or homologous small molecules, isolated from, or substantially free of contaminating molecules, as treatment of a chronic disease.

The following sections present standard protocols for formulation of small molecules.

(a) Production

Small molecules, organic or inorganic, may be synthesized in vitro by any of a number of methods well known in the art. Those small molecules, and others synthesized in vivo, may by purified by, for instance, liquid or thin layer chromatography, high performance liquid chromatography (HPLC), electrophoresis, or some other suitable technique.

(3) Polypeptides

Another aspect of the invention pertains to administration of a polypeptide of interest, equivalent polypeptides, or homologous polypeptides, isolated from, or substantially free of contaminating molecules, as treatment of a chronic disease.

The following sections present standard protocols for the formulation of polypeptides.

(a) Production

(i) In vitro

In one exemplary embodiment, a polypeptide of interest is produced in vitro by introducing into a host cell by any of a number of means well known in the art (see protocols below) a recombinant expression vector carrying a polynucleotide, preferably obtained from vertebrates, especially mammals, encoding a polypeptide of interest, equivalents of such polypeptide, or homologous polypeptides. The recombinant polypeptide is engineered to include a tag to facilitate purification. Such tags include fragments of the GST protein, or polyamino acid tracts either recognized by specific antibodies, or which convey physical properties facilitating purification (see also below). Following culture under suitable conditions, the cells are lysed and the expressed polypeptide purified. Typical culture conditions include appropriate host cells, growth medium, antibiotics, nutrients, and other metabolic byproducts. The expressed polypeptide may be isolated from a host cell lysate, culture medium, or both depending on the expressed polypeptide. Purification may involve any of many techniques well known in the art, including but not limited to, gel filtration, affinity chromatography, gel electrophoresis, ion-exchange chromatography, and others.

Polynucleotides, both mRNA and DNA, can be extracted from prokaryotic or eukaryotic cells, or whole animals, at any developmental stage, for instance, adults, juveniles, or embryos. Polynucleotides may be isolated, or cloned from a genomic library, cDNA library, or freshly isolated nucleic acids, using protocols well known in the art. For instance, total RNA is isolated from cells, and mRNA converted to cDNA using oligo dT primers and viral reverse transcriptase. Alternatively, a polynucleotide of interest may be amplified using PCR. In any case, the initial nucleic acid preparation may include either RNA or DNA and the protocols chosen accordingly. The resulting DNA is inserted into an appropriate vector, for instance, bacterial plasmid, recombinant virus, cosmid, or bacteriophage, using procedures well known in the art.

Nucleotide sequences are considered functionally linked if one sequence regulates expression of the other. To facilitate expression of a polypeptide of interest, the cloning vector should include suitable transcriptional regulatory sequences well known in the art, for instance, promoter, enhancer, polyadenylation site, etc., functionally linked to the polynucleotide expressing the polypeptide of interest. In one exemplary embodiment, an expression vector is constructed to carry a polynucleotide, a naturally occurring sequence, a gene, a fusion of two or more genes, or some other synthetic variant, under control of a regulatory sequence, such that when introduced into a cell expresses a polypeptide of interest.

Both viral and nonviral gene transfer methods may be used to introduce desirable polynucleotides into cells. Viral methods exploit natural mechanisms for viral attachment and entry into target cells. Nonviral methods take advantage of normal mammalian transmembrane transport mechanisms, for example, endocytosis. Exemplary protocols employ packaging of deliverable polynucleotides in liposomes, encasement in synthetic viral envelopes or poly-lysine, and precipitation with calcium phosphate (see also below).

The variety of suitable expression vectors is vast and growing. For example, mammalian expression vectors typically include prokaryotic elements which facilitate propagation in the laboratory, eukaryotic elements which promote and regulate expression in mammalian cells, and genes encoding selectable markers. The list of appropriate vectors includes, but is not limited to, pcDNA/neo, pcDNA/amp, pRSVneo, pZlPneo, and a host of others. Many viral derivatives are also available, for instance, pHEBo, derived from the Epstein-Barr virus, BPV-a derived from the bovine papillomavirus, and the pLRCX system (BD Biosciences Clontech, Inc.). The use of mammalian expression vectors is well known in the art (see, for example, Sambrook 2001, ibid, chapters 15 and 16). Similarly, many vectors are available for expression of recombinant polypeptides in yeast, including, but not limited to, YEP24, YEP5, YEP51, pYES2. The use of expression vectors in yeast is well known in the art.

In addition to mammalian and yeast expression systems, a system of vectors is available which permits expression in insect cells. The system, derived from baculoviruses, includes pAcUW-based vectors (for instance, pAcUW1), pVL-based vectors (for instance, pVL1292 and pVL1393), and pBlueBac-based vectors which carry the gene encoding β-galactosidase to facilitate selection of host cells harboring recombinant vectors.

(ii) In situ

In another exemplary embodiment, a polypeptide of interest is expressed in situ by administering to an animal or human subject by any of a number of means well known in the art (see protocols below) a recombinant expression vector carrying a polynucleotide encoding the polypeptide of interest, equivalent polypeptides, or homologous polypeptides.

In the present invention, such vectors may be used as therapeutic agents to introduce polynucleotides into cells that express constructive or disruptive polypeptides (for exemplary applications see, for instance, Friedmann 1999¹³⁴).

It is critical that the potential effects of microcompetition between the enhancer, or other polynucleotide sequences carried in the delivery vector, and cellular genes be considered and manipulated where needed. As an example consider a case where the polypeptide of interest binds an enhancer carried by the vector, for instance, a delivery vector that expresses GABP under control of a promoter that includes an N-box. In one exemplary embodiment, the vector expresses, in situ, a high enough concentration of the polypeptide of interest such that any binding of the polypeptide to the enhancer sequences within the vector itself is negligible. In other words, the vector expresses enough free polypeptides to produce the desired biological activity in treated cells. In another example, the polypeptide is not a transcription factor, but the delivery vector carries a polynucleotide that microcompetes with cellular genes for a cellular transcription factor, for instance, a vector that expresses Rb and microcompetes with cellular genes for GABP. In an exemplary embodiment, the delivery vector also includes a polynucleotide sequence that expresses the microcompeted transcription factor, or is delivered in conjunction with another vector that expresses the microcompeted transcription factor. In the example, the Rb vector includes a sequence that expresses GABP, or is delivered in conjunction with a vector that expresses GABP.

(4) Polynucleotides

Another aspect of the invention pertains to administration of a polynucleotide as antisense/antigene, ribozyme, triple helix, homologous nucleic acids, peptide nucleic acids, or microcompetitiors, equivalent polynucleotides, or homologous polynucleotides, isolated from, or substantially free of contaminating molecules, as treatment for a chronic disease.

The following sections present standard protocols for the formulation of such polynucleotides. Since antisense/antigene, ribozyme, triple helix, homologous nucleic acids, peptide nucleic acids, and microcompetition agents are nucleic acid based, they share protocols for their synthesis, mechanisms of delivery and potential pitfalls in their use including, but not limited to, susceptibility to extracellular and intracellular nucleases, instability and the potential for nonspecific interactions. In consideration of these common issues, the general methods for the formulation and delivery, as well as caveats regarding the use of nucleic agents, described first, apply similarly to each subsequent agent.

(a) Antisense/Antigene

In the present invention, the terms “antisense” and “antigene” polynucleotides is understood to include naturally or artificially generated polynucleotides capable of in situ binding to RNA or DNA, respectively. Antisense binding to mRNA may modify translation of bound mRNA, while antigene binding to DNA may modify transcription of bound DNA. Antisense/antigene binding may modify binding of a polypeptide of interest to RNA or DNA, for instance binding of an antigene to a foreign N-box may reduce binding of cellular GABP to the foreign N-box resulting in attenuated microcompetition between the foreign polynucleotide and a cellular gene for GABP. Antisense/antigene binding may also modify, i.e., decrease or increase, expression of a polypeptide of interest.

Binding, or hybridization of the antisense/antigene agent, may be achieved by base complementarity, or by interaction with the major groove of the cellular DNA duplex. The techniques and conditions for achieving such interactions are well known in the art. The target of antisense/antigene agents has been thoroughly studied and is well known in the art. For instance, the antisense preferred target is the translational initiation site of a gene of interest, from approximately 10 nucleotides upstream to approximately 10 nucleotides downstream of the translational initiation site. Oligonucleotides targeting the 3′ untranslated mRNA regions are also effective inhibitors of translation. Therefore, oligonucleotides targeting the 5′ or 3′ UTRs of a polynucleotide of interest may be used as antisense agents to inhibit translation. Antisense agents targeting the coding region are less effective inhibitors of translation but may be used when appropriate.

Effective synthetic agents are typically between 20 and 30 nucleotides in length. However, to be effective, a complementary sequence must be sufficiently complementary to bind tightly and uniquely to the polynucleotide of interest. The degree of complementarity is generally understood by those skilled in the art to be measured relative to the length of the antisense/antigene agent. In other words, three bases of mismatch in a 20 base oligonucleotide have a more profoundly detrimental effect than three bases of mismatch in a 100 base oligonucleotide. Inadequate complementarity results in ineffective inhibition, or unwanted binding to sequences other than the polynucleotide of interest. In the latter case, inadvertent effects may include unwanted inhibition of genes other than a gene of interest. Specificity and binding avidity are easily determined empirically by methods known in the art.

Several methods are suitable for the delivery of antisense/antigene agents. In one exemplary embodiment, a recombinant expression plasmid is engineered to express antisense RNA following introduction into host cells. The RNA is complementary to a unique portion of DNA or mRNA sequence of interest. In an alternative embodiment, chemically derivatized synthetic oligonucleotides are used as antisense/antigene agents. Such oligonucleotides may contain modified nucleotides to attain increased stability once exposed to cellular nucleases. Examples of modified nucleotides include, but are not limited to, nucleotides carrying phosphoramidate, phosphorothioate and methylphosphonate groups.

Whichever sequence of the polynucleotide of interest is targeted by antisense/antigene agents, in vitro studies should be undertaken first to determine the effectiveness and specificity of the agent. Control treatments should be included to differentiate between effects specifically elicited by the agent and non-specific biological effects of the treatment. Control polynucleotides should have same length and nucleotide composition as the agent with the base sequence randomized.

Antisense/antigene agents can be oligonucleotides of RNA, DNA, mixtures of both, chemical derivatives of either, and single or double stranded. Nucleotides within the oligonucleotide may carry modifications on the nucleotide base, the sugar or the phosphate backbone. For example, modifications to the nucleotide base involves a number of compounds including, but not limited to, hypoxanthine, xanthine, 2-methyladenine, 2-methylguanine, 7-methylguanine, 5-fluorouracil, 3-methylcytosine, 2-thiocytosine, 2-thiouracil, 5-methylcytosine, 5-methylaminomethyluracil, and a host of others well known in the art. Modifications are generally incorporated to increase stability, e.g. infer resistance to cellular nucleases, stabilize hybridization, or increase solubility of the agent, increased cellular uptake, or some other appropriate action.

In a related exemplary embodiment, adducts of polypeptides, to target the agent to cellular receptors in vivo, or other compounds which facilitate transport into the target cell are included. Additional compounds may be adducted to the antisense/antigene agent to enable crossing of the blood-brain barrier, cleavage of the target sequence upon binding, or to intercalate in the duplex which results from hybridization to stabilize that complex. Any such modification, intended to increase effectiveness of the antisense/antigene agent, is included in the present invention.

Similarly, the antisense/antigene agent may include modifications to the phosphate backbone including, but not limited to, phosphorothioates, phosphordamidate, methylphosphonate, and others. The agent may also contain modified sugars including, but not limited variants of arabinose, xylulose and hexose.

In another exemplary embodiment, the antisense/antigene agent is an alpha anomeric oligonucleotide capable of forming parallel, rather than antiparallel, hybrids with a cellular mRNA of interest.

It is common for antisense agents to be targeted against the coding regions of an RNA of interest to effect translational inhibition. In a preferred embodiment, antisense agents are targeted instead against the transcribed but untranslated region of an RNA transcript. In this case, rather than achieving translational inhibition, it is likely that oligonucleotides hybridized to the target transcript will lead to mRNA degradation through a pathway mediated by RNaseH or similar cellular enzymes.

For optimal efficacy, the antisense/antigene agents must be delivered to cells carrying the polynucleotide of interest in vivo. Several delivery methods are known in the art, including but not limited to, targeting techniques employing polypeptides linked to the antisense/antigene agent which bind to specific cellular receptors. In this instance the agents may be provided systemically. Alternatively the agents may be injected directly into the tissue of interest, or packaged in a virus, including retroviruses, chosen because its host range includes the target cell. In every case, the agent must enter the target cell to be effective.

Antisense/antigene methodologies often face the problem of achieving sufficient intracellular concentration of the agent to effectively compete with cellular transcription and/or translation factors. To overcome this challenge, those skilled in the art introduce recombinant expression vectors carrying the antisense/antigene agent. Once introduced into the target cell, expression of the antisense/antigene agent from the incorporated RNA polymerase II or III promoter results in sufficient intracellular concentrations. Vectors can be chosen to integrate into the host cell chromosomes, thereby becoming stable through multiple rounds of cell division, or vectors may be used which remain unintegrated and therefore are lost when the target cell divides. In either case, the primary goal is attaining levels of transcription that produce sufficient antisense/antigene agents to be effective. The choice of a suitable vector and the development of an effective antisense construct involves techniques standard in the art.

Antisense/antigene expression man be regulated by any promoter known to be active in mammalian, especially human, cells and may be either constitutively active or inducible. Regardless of the promoter chosen, it is important to test for the effect of any enhancer regions intrinsic to those promoters as they may participate in microcompetition with cellular genes. In the case of inducible promoters, the biological effects of the expressed antisense can be discerned from any effect the promoter has on microcompetition by assaying any bioactivity with and without induced gene expression. Suitable promoters, inducible or not, are well known in the art (see, for example, Jones 1998¹³⁵).

Antisense agents may be prepared using any of a number of methods commonly known to those skilled in the art. In on exemplary embodiment, oligonucleotides, up to approximately 50 nucleotides in length, may be synthesized using automated processes employing solid phase, e.g. controlled pore glass (CPG) technology, such as that used on the Applied Biosystems model 394 medium throughput synthesizer, or 5′-phosphate ON (cyanoethyl phosphoramidite) chemistry developed by Clonotech Laboratories, Inc. In each of these procedures, oligonucleotides are synthesized from a single nucleotide using a series of deprotection and ligation steps. The underlying chemistry of the reactions is standard practice and the availability and accessibility of automated synthesizers bring these synthetic technologies within the grasp of anyone skilled in the art.

Despite the ease of synthesis, the selection of effective antisense agents involves the identification of a suitable target for the agent. This process is simplified somewhat by the many software programs available, such as, for example, Premier Primer 5, available from Premier Biosoft International or Primer 3, available online at http://www-genome.wi.mit.edu/cgi-bin/primer/primer3.cgi. Alternatively, a scientist skilled in the art may design antisense agents manually. Relevant aspects of the design process which need attention include selection of the target region to which the antisense agent will bind. Ideally it will be the gene promoter, if the target is DNA, or the translation initiation site if the target is an mRNA. Attention also needs to be paid to the length of the agent, typically at least 20 nucleotides are needed for specificity. Shorter oligonucleotides carry the risk of non-specific binding and therefore may lead to undesired side effects. Also, the agents must be composed of a sequence that will not promote hybridization between the oligonucleotides in the agent during application. Taken together, these considerations are well known and are addressed by standard procedures well known in the art.

Longer antisense agents may be produced within the target cell from recombinant expression vectors. In one exemplary embodiment, the desired antisense-encoding sequences can be incorporated into an appropriate expression vector selected because it contains the regulatory sequences necessary to ensure expression in the target cell type. Selection of the sequence composition of the antisense agent must take into account the same considerations used to design shorter oligonucleotides as described in the previous paragraph including, but not limited to, binding specificity for the target sequence and minimizing interactions between the expressed agents. Techniques for the design and construction of appropriate recombinant expression vectors are well known to those skilled in the art.

Control agents, whether synthetic oligonucleotides or longer antisense agents expressed in vivo by expression vectors, are employed to validate the efficacy and specificity of the therapeutic agents. Each control agent should have the same nucleotide composition and length as the therapeutic agent but the sequence should be random. Employment of this agent will permit the determination of whether any effects observed after treatment with the therapeutic agent are indeed specific. Specificity will reduce the potential for binding to targets other than those desired, thereby reducing associated unwanted side effects.

Purification of Oligonucleotides: The efficacy of synthetic oligonucleotide agents is impacted by their purity. Under typical conditions, approximately 75% of the synthesis products are full length while the remaining 25% of the oligonucleotides are shorter. This proportion of full length to shorter products varies with the length of the desired product. The synthesis of longer oligonucleotides is less efficient, and therefore the synthesis products contain a smaller proportion of full-length products, than that of shorter ones. Unwanted, shorter synthesis products have reduced specificity compared to the full length products and are therefore undesirable in a therapeutic formulation due to their reduced specificity which in turn leads to an increased risk of side effects.

In one exemplary embodiment, full-length oligonucleotides greater than 50 bp in length are purified by virtue of their size. Gel permeation chromatography is used to separate full-length products from the shorter synthetic byproducts. In a complementary exemplary embodiment, full length synthetic oligonucleotides shorter than 50 bp may be purified by liquid chromatography using charged resins such as hydroxyapatite or nucleic acid specific resins such as RPC-5 (which is composed of trioctylmethylamine adsorbed onto hydrophobic plastic particles). This latter technique exploits both hydrophobic and ion exchange methods to achieve high reagent purity and is amenable to use in HPLC.

Regardless of the method of purification used, the desired oligonucleotides are concentrated by precipitation with ice-cold ethanol followed by lyophilization and dissolution in an appropriate carrier for treatment. Carrier selection is another important component of agent formulation. It is essential that the carrier used be first tested for biological activity in the target cell type. This control measure, well known to those skilled in the art, will ensure that any effects observed upon administration of the nucleic acid agent are indeed due to the agent and not the carrier in which it is administered (on purification of oligonucleotides see, for instance, Deshmukh (1999¹³⁶).

Delivery of Oligonucleotides: Methods for effective administration of antisense agents vary with the agent used. In one exemplary embodiment, synthetic oligonucleotides are delivered by simple diffusion into the target cells. Advantages of this delivery method include the ability to administer the agent systemically, for example by intravenous injection. This method, while effective carries several risks, not the least of which is the potential to introduce oligonucleotides into cells other than those of the desired target. Another disadvantage involves the risk of degradation by nucleases in blood and interstitial fluid. This second disadvantage may be partially avoided by modification of the synthetic oligonucleotide in such a way, for example by incorporated modified nucleotides such as those carrying phosphorothioate or methyl phosphonate moieties, which renders them relatively resistant to exonuclease degradation.

In a related embodiment those same agents may be delivered by way of liposome mediated transfection as described by Daftary and Taylor (2001¹³⁷). This method enhances diffusion into the target cell by encasing the antisense agent in a lipophilic liposome. However, this method too has drawbacks. While cellular uptake is enhanced, the ratio of liposome components to DNA must be carefully controlled in order to maximize delivery efficiency. This technique is commonly employed and is well known to those skilled in the art.

In another exemplary embodiment, antisense expressing viral vectors may be used to confer target cell specificity. In some cases, viral delivery agents may be selected which include the target cell type in their respective host range. This delivery method minimizes unwanted side effects that otherwise may arise from delivery of the therapeutic agent to the incorrect cell type. However, this advantage may be negated if the multiplicity of infection is too high and non-specific infection is thereby promoted. This potential problem may be avoided by thoroughly testing any viral deliver agent, using techniques well known in the art, prior to its clinical administration.

(b) Ribozymes

While antisense agents act by either inhibiting transcription or translation of the target gene, or by inducing enzyme-mediated transcript degradation by RNase H or a similar enzyme, ribozymes offer an alternative approach. Ribozymes are RNA molecules which natively bind to and cleave target transcripts. Typical ribozymes bind to and cleave RNA at specific sites, however hammerhead ribozymes cleave target transcripts at sites directed by flanking nucleotide sequences which bind to the target site. The use of hammerhead ribozymes is preferred because the only sequence requirement for their activity is the UG dinucleotide arranged in the 5′-3′ orientation. Hammerhead technologies are well known in the art (see, for example Doherty 2001¹³⁸, or Goodchild 2000¹³⁹). In a preferred embodiment, the sequence targeted by the ribozyme lies near the 5′ end of the transcript. That will result cleavage of the transcript near the translation initiation site thereby blocking translation of a full-length protein.

Ribozymes identified in Tetrahymena thermophila, which employ an eight base pair active site which duplexes with the target RNA molecule, are included in this invention. This invention includes those ribozymes, described and characterized by Cech and coworkers (i.e. IVS or L-191VS RNA), which target eight base-pair sequences in a gene of interest and any others which may be effective in inhibiting expression of a disrupted gene or a gene in a disrupting pathway. For the catalytic sequence of these agents see, for instance, U.S. Pat. No. 5,093,246, incorporated entirely herein by reference. Any ribozyme or hammerhead ribozyme molecules that target RNA sequences expressed by a foreign polynucleotide, disrupted gene or gene in a disrupted pathway, are included in this invention.

Ribozymes, being RNA molecules of specific sequence, may be synthesized with modified nucleotides which enable better targeting to the host cell of interest or which improve stability. As described above for conventional antisense agents, the preferred method of delivery involves introduction into the target cell, a recombinant expression vector encoding the ribosome. Inclusion of an appropriate transcriptional promoter will ensure sufficient expression to cleave and disrupt transcripts of foreign DNA or disrupted genes or genes in a disrupting pathway. The catalytic nature of ribozymes permits their effective use at concentrations below those needed for traditional antisense agents.

Identification of ribozyme cleavage sites within a transcript of interest is accomplished with any of a number of computer algorithms which scan linear oligonucleotide sequences for alignments with a query sequence. The identified sequence, commonly containing the trinucleotide sequences GUC, GUA or GUU, will serve as the nucleus of a longer sequence of approximately 20 nucleotides in length. That longer sequence will be examined, again with appropriate computer algorithms well known in the art, for their potential to form secondary structures which may interfere with the action of targeted ribozyme agents. Alternatively, empirical assays employing ribonucleases may be used to probe the accessibility of identified target sequences.

Ribozymes comprise a unique class of oligonucleotides which bind to specific ribonucleic acid targets and promote their hydrolysis. The design of ribozyme agents is well known to those skilled in the art. In order to prepare effective ribozyme agents, initially a suitable target sequence must be identified which confers specificity to the agent in order to minimize unwanted side effects and maximize efficacy. Once that target is identified the ribozyme agent is synthesized using standard oligonucleotide synthesis procedures such as those exemplified herein. Delivery to the target cell may be accomplished by direct transfection ex vivo or by liposome-mediated transfection.

Ensuring the purity and efficacy of ribozyme agents may be more important than for other nucleic acid agents because their intended effects, namely the hydrolysis of target sequences, are irreversible. In this light extensive preclinical testing is essential to minimize unwanted side effects. These risks are, however, outweighed by the potential effectiveness of ribozyme agents.

(c) Triple Helix

In a related embodiment, synthetic single-stranded deoxyribonucleotides can be chosen which form triple helices according to the Hoogsteen base pairing rules. The rules necessitate long stretches of either purines or pyrimidines on one strand of the DNA duplex. In either case, triplexes are formed, with pyrimidines pairing with purines within the target sequence and vice versa, which inhibit transcription of the target sequence. The effectiveness of a targeted triplex forming oligonucleotide may be enhanced by including a “switchback” motif composed of alternating 5′-3′ and 3′-5′ regions of purines and pyrimidines. This “switchback” reduces the length of the required purine or pyrimidine tract in the target because the oligonucleotide can form duplexes alternatively with each strand of the target sequence.

Triple helix forming agents are oligonucleotides which have been designed to interact with cellular nucleic acids and form triple helices. The resulting structure may be targeted by intracellular degradation pathways or may provide a steric block to nucleic acid replication, transcription or translation depending on the target.

Triplex agent formulation begins with selection of an appropriate target sequence within the cells to be treated. That target may be within the cellular DNA or RNA or within that of an exogenous source such as an infecting virus. Suitable target sequences should contain long stretches of homopyrimidines or homopurines and the most effective targets contain alternative stretches of each. If the target is double stranded DNA, the most effective targets surround and include the transcriptional regulatory regions. Formation of a triplex between the agent and the target will inhibit the binding of RNA polymerase or other requisite transcriptional regulatory factors which otherwise bind the promoter and upstream regulatory regions.

Triplex agents may be synthesized to be more resistant to cellular and extracellular nucleases by the inclusion of modified nucleotides such as those containing phosphorothioate or methyl phosphonate groups. In the event that such modifications interfere with base pairing, additional adducts, such as derivatives of the base intercalating agent acridine, may be incorporated into the therapeutic agent to restore desirable binding properties to the triplex forming oligonucleotide. Alternatively, if the intracellular target is an mRNA, C-5 propyne pyrimidines may be included in the synthetic oligophosphorothioate agent to increase its binding affinity for mRNA and therefore decrease the concentration required for effectiveness.

The affinity of triplex agents for their respective targets may be assessed by electrophoretic gel retardation assays. The formation of triplex structures will retard migration through an electrophoretic gel. Similarly, UV melting experiments can assess the stability of any triplex agent binding to its target. In these assays triplex agents are mixed with their intended target in vitro and the resulting triplexes are heated (with, for example, a Haake cryothermostat) while monitoring their UV absorbance (with, for example, a Kontron-Uvikon 940 spectrophotometer) (on design of triplex forming oligonucleotides see, for instance, Francois (1999¹⁴⁰)).

Triplex forming agents are simply oligonucleotides designed to form triple helices with the target intracellular nucleic acid. Accordingly, their synthesis, purification and delivery parallels the procedures described herein for other oligonucleotide agents. Each of these processes is commonly known to those skilled in the art.

(d) Homologous Recombination Agents

Binding of factors to foreign polynucleotides (either DNA or RNA), or polynucleotides of disrupted genes, or polynucleotides of a gene in a disrupted or disrupting pathway, or expression of a foreign gene, or a disrupted gene, or a gene in a disrupted or disrupting pathway can also be reduced by mutating the DNA, inactivating, or “knocking out” the gene or its promoter using targeted homologous recombination.

In one exemplary embodiment, a polynucleotide of interest flanked by DNA homologous to the polynucleotide interest (encompassing either the coding or regulatory regions of the polynucleotide) can be introduced into cells carrying the same sequence. Homologous recombination mediated by the flanking sequences disrupts expression of the polynucleotide of interest and result in reduced expression. The technique is frequently used by those skilled in the art to engineer transgenic animals that produce offspring with same disruption. However, the same approach may be used in humans by administering the engineered construct into target cells. Regardless of expression vector platform chosen, it is important to recognize and control for any microcompetition effects that may be elicited by transcriptional enhancers carried by the viral vectors (see also above). Control experiments must be carried out which study the biological activity of a non-recombinant viral vector to reveal any effects its intrinsic enhancers have on the target biological activities.

Nucleic acid agents for homologous recombination are designed to interact with specific cellular DNA targets and undergo recombination. The specificity of the therapeutic agent is conferred by the nucleotide sequences at its termini, they must be complementary to adjacent cellular targets and bind them through Watson-Crick base pairing.

Formulation of these agents involves careful selection of the desired cellular target. The nucleotide sequence of that target must be available in public or private sequence databases. The agent itself may be comprised of a synthetic oligonucleotide or a recombinant nucleic acid carried in a suitable vector.

In one exemplary embodiment, a synthetic oligonucleotide may be used for homologous recombination in order to interrupt the coding sequence or regulatory sequences of the target gene. The oligonucleotide is designed to include nucleotides at its termini which are complementary to those of the target sequence and the central regions may contain any sequence that is neither complementary to the target sequence nor carry an in-frame insertion into the target sequence.

In a related embodiment, a longer sequence of nucleic acid may be used. The sequence of interest, which is intended to either interrupt a cellular gene or insert additional coding capacity into it, is flanked by sequences homologous to the cellular target. That entire DNA fragment is then inserted into an appropriate prokaryotic or viral vector for delivery to the target cells. Once inside the cell the agent will bind to and recombine with the target gene.

(e) Peptide Nucleic Acids

In various embodiments, hybridization of the nucleic acid agents described herein may be enhanced by the substitution of amino acids for the deoxyribose of the nucleic acid backbone, thereby creating peptide nucleic acids (see, for example, Hyrup 1996¹⁴¹). This modification leads to a reduction of the overall negative charge on the backbone and therefore reduces the need for counter ions to permit sequence-specific hybridization of two strands of negatively charged polynucleotides. Peptide nucleic acids can be synthesized using techniques well known in the art such as the solid phase protocols described by Hyrup and Nielsen (1996, ibid), and Perry-O'Keefe 1996, included herein in their entirety by reference.

Oligonucleotides so modified can be used in the same therapeutic techniques as unmodified homologs. They can be used as antisense agents designed to interfere with the expression of a foreign polynucleotide, a disrupted gene, or a gene in a disrupted pathway. Similarly, by virtue of their enhanced hybridization qualities, peptide nucleic acids can be used, for example, as primers for the PCR, for S1 nuclease mapping of single stranded regions and for other enzyme-based techniques. Similarly, peptide nucleic acids may be modified by the addition of lipophilic moieties to enhance the cellular uptake of therapeutic oligonucleotide agents. In related embodiments, peptide nucleotide agents may be synthesized as chimeras comprised of peptide nucleic acids and unmodified DNA. This configuration exploits the advantages of a peptide nucleic acid while the DNA portion of the molecule can serve as a substrate for cellular enzymes.

Peptide Nucleic Acid (PNA) is a DNA analog in which the sugar-phosphate backbone contains a pseudopeptide rather than the sugars characteristic of DNA. Like DNA, PNA agents bind complementary nucleic acid strands thereby mimicking the behavior of DNA. This activity is enhanced by the neutral, rather than negatively charged, backbone of PNA, which promotes more tenacious and more specific binding than that of DNA. These are among many favorable properties of PNA and include, in addition, increased stability and exhibit improved hybridization properties compared to their DNA analogs. While the mechanism of PNA action is currently not fully understood, for example PNA-RNA hybrids are not targets for RNase H degradation as are DNA-RNA hybrids, it is likely that they inhibit translation by blocking the binding of RNA polymerase or other critical factors to the target mRNA.

In this light, it is important to select targets that include the translation initiation codon. Other target sites further downstream on the mRNA may be effective at inhibiting translation by interfering with ribosome transit although the role of this activity will need to be determined empirically for each agent developed. In any case the actual mechanism of action, while interesting, is not necessary to ascertain as long as the agent is effective and does not induce undesired side effects.

Homopurines are best targeted by homopyrimidine PNAs with stretches of greater than 8 bp providing suitable targets within double stranded DNA. The synthesis of PNA agents is achieved using automated solid-phase techniques employing Boc-, Fmoc- or Mmt-protected monomers. Alternatively, commercial sources of custom synthetic PNAs, including Applied Biosystems (Foster City, Calif.) may be exploited to minimize in-house expenses and expertise (on design of PNA see, for instance, Nielsen 1999¹⁴³).

(5) Antibodies and Antigens

Another aspect of the invention pertains to the administration of an antibody of interest, equivalent of such antibody, homolog of such antibody, as treatment of a chronic disease.

For example, using standard protocols, one skilled in the art can use immunogens derived from a foreign polynucleotide, foreign polypeptide, disrupted gene, disrupted polypeptide, gene or polypeptide in a disruptive or disrupted pathway, to produce anti-protein, anti-peptide antisera, or monoclonal antibodies (see, for example, Harlow and Lane 1999¹⁴⁴, Sambrook 1989¹⁴⁵).

Animals, which have been injected with an immunogenic agent, can serve as sources of antisera containing polyclonal antibodies. Monoclonal antibodies, if desired, may be prepared by isolating lymphocytes from the immunized animals and fusing them, in vitro with immortal, oncogenically transformed cells. Clonal lines from the resulting somatic cell hybrids, or hybridomas, can be used as sources of monoclonal antibodies specific for the immunogen of interest. Techniques for developing hybridomas and for isolating and characterizing monoclonal antibodies are well known in the art (see for instance, Kohler 1975¹⁴⁶ and Zola 2000¹⁴⁷).

In the context of this invention, “antibody” refers to entire molecules or their fragments, which react specifically with polypeptides or polynucleotides of interest, whether they are monospecific, bispecific or chimeras that recognize more than two antigenic determinants. Those skilled in the art employ well-known methods for producing specific antibodies and for fragmenting them. While several methods are known to produce antibody fragments, pepsin, for example, is used to treat whole antibody molecules to produce F(ab)₂ fragments. These fragments can be further dissociated with chemicals, such as beta mercaptoethanol or dithiothreotol, which reduce intra and intermolecular disulfide bridges resulting in the release of Fab fragments.

Once produced, isolated and characterized, antibodies, or fragments thereof, which bind to antigenic determinants of interest may be used for diagnostic and analytical purposes. For example, they may be used in immunohistochemical assays to assess expression levels of polynucleotides or polypeptides of interest. They may also be employed in other immunoassays, including but not limited to, Western blots, immunoaffinity chromatography, and immunoprecipitation carried out to quantify protein levels in cells or tissues of interest. The assays, individually or together, may also be used by one skilled in the art to measure the concentration a protein of interest before and after therapy to assess therapeutic efficacy.

Similarly, it is common in the art to use specific antibodies to screen libraries of recombinant expression vectors for those expressing a protein or polypeptide of interest. Suitable expression vectors are commonly derived from bacteriophage, including, for example, λgt11 and its derivatives. Identification of expression vectors, from among a library of similar recombinants, can lead to the identification of vectors expressing a polypeptide of interest which may then itself be used in diagnostic or therapeutic assays. In a preferred embodiment, antibodies specific for a particular polypeptide, protein or antigenic determinant carried thereon, will cross-react with homologous counterparts from different species to facilitate antibody characterization and assay development.

Antibodies may serve as effective therapeutic agents for the inactivation of specific cellular proteins or for targeting other therapeutic agents to cells expressing particular surface antigens to which an antibody may bind. Polyclonal antibodies are prepared in a suitable host organism, typically rabbit, goat or horse, by injecting the appropriate purified antigen into the host. Following a regimen of repeated challenges by the desired antigen, using protocols well known to those skilled in the art, serum is drawn from the host and assayed for the presence of antibodies. Once a suitable response is detected, additional serum is removed, perhaps leading to exsanguination of the producing organism, and the desired antibodies are purified.

Monoclonal antibodies may be prepared by any number of techniques well known to those skilled in the art. In one exemplary embodiment, cells expressing the desired target antigen are fused with immortalized cells in vitro. The resulting hybridomas are cultured and clonal lines are derived using standard tissue culture techniques. Each resulting clone is assayed for expression of antibodies against the desired antigen, typically but not necessarily by ELISA.

Antibodies may be purified by a number of chromatographic techniques. In one exemplary embodiment, antibodies may be bound to S. aureus protein A cross-linked to a suitable support resin (e.g. sepharose). The crude antibody preparation is slowly applied to the chromatographic column under conditions that permit antibody-protein A interactions. The resin is then washed with several column volumes of buffer to remove adventitiously bound and trapped proteins, leaving only specifically bound antibodies on the column. Those are eluted by washing the column with 100 mM glycine (pH 3.0) and monitoring protein elution spectrophotometrically.

In an alternative embodiment, antibodies are purified by binding to an affinity column comprised of antigen cross-linked to an appropriate solid support. Bound antibodies may be eluted by any of a number of methods and may include the use of an elution buffer containing glycine at low (e.g. 3.0) pH or 3M potassium thiocyanate and 0.5M NH₄OH. Due to the varied mechanisms involved with antibody-antigen interactions, the actual optimal elution conditions must determined empirically.

The therapeutic efficacy of polyclonal compared to monoclonal antibodies cannot be predicted. Each has strengths and weaknesses. For example, polyclonal antibodies necessarily target multiple antigenic determinants on the target antigen. This feature may increase reactivity but, at the same time, may decrease specificity. On the other hand, monoclonal antibodies are exquisitely specific for a single antigenic determinant on the target antigen. This specificity greatly reduces the risk of unwanted reactivity with other antigens, and the associated side effects, yet carries the risk that the target antigenic determinant may be inaccessible in the cellular environment, either due to the natural folding of the protein or through interactions with other cellular molecules. In every case, the efficacy of any antibody agent must be determined empirically using a variety of techniques well known to those skilled in the art.

Antibody production is necessarily preceded by the isolation and purification of appropriate antigens. Cellular proteins may be purified by any of a number of techniques well known to those skilled in the art. In one exemplary embodiment, cells expressing the desired antigen are lysed in the presence of non-ionic detergents and the resulting lysate is subjected to purification. That lysate is then fractionated by precipitation in the presence of ammonium sulfate. Sequentially higher concentrations of ammonium sulfate are used to derive protein mixtures that differ by their solubility in ammonium sulfate. Each fraction is then assessed for the presence of the desired antigen.

The fraction carrying the protein of interest is subjected to further purification by any of a number of well-known methods. For instance, if an antibody against the protein is available, the protein may be purified by affinity chromatography using a resin of substrate, typically sepharose, dextran or some similar insoluble polymer, to which the antibody is conjugated. The protein mixture containing the desired antigen is exposed to the resin under conditions that promote antibody-antigen interactions. Adventitiously bound proteins are washed from the resin with an excess of binding buffer and the antigens are eluted with buffer containing an ionic detergent such as sodium dodecylsulfate (SDS).

In an alternative embodiment, crude fractions of cellular proteins are further purified using methods well known in the art involving ion exchange or molecular exclusion chromatographic techniques. The purity of antigens isolated by any technique may be assessed by electrophoresis through denaturing polyacrylamide gels followed by visualization by staining.

c) Assay Protocols

One aspect of the invention pertains to assaying the effect of an agent on a molecule of interest, equivalent molecules, or homologous molecules during drug discovery, development, use as treatment, or during diagnosis.

(1) Definitions

(a) Molecule of Interest

The term “molecule of interest” is understood to include, but not limited to, p300/cbp, p300/cbp polynucleotides, p300/cbp factors, p300/cbp regulated genes, p300/cbp regulated polypeptides, p300/cbp factor kinases, p300/cbp factor phosphatases, p300/cbp agents, foreign p300/cbp polynucleotides, p300/cbp viruses, disrupted genes, disrupted polypeptides, genes in disrupted pathways, polypeptides in disrupted pathways, genes in disruptive pathways, polypeptides in disruptive pathways.

Every gene and protein mentioned in this invention is uniquely defined by its sequence as published in public databases. See, for instance, the sequences in the nucleotide and protein sequence databases at NCBI (also known as Entrez, the name of the search and retrieval system), GenBank, the NIH genetic sequence database, DDBJ, the DNA DataBank of Japan, EMBL, the European Molecular Biology Laboratory database (GenBank, DDBJ and EMBL comprise the International Nucleotide Sequence Database Collaboration), SWISS-PROT, the protein knowledgebase, and TrEMBL, the computer-annotated supplement to SWISS-PROT (see also the search and retrieval system Expasy), PROSITE, the database of protein families and domains, and TRANSFAC, the database of transcription factors. By a gene it is meant the coding and non-coding regions, the promoters, enhancers, and the 5′ and 3′ UTRs. Published sequences are considered standard information and are well known in the art. In one exemplary embodiment, sequences for certain genes and proteins of interest in this invention are listed in the following section. For most genes, the list includes the human sequence. However, homologous sequences (see definition below) are available in the above databases for other organisms, such as mouse, rat, etc. The following listed sequences should be regarded as illustrations, and, therefore, should not be construed as limiting the invention in any way.

List of Sequences

Metallothionein IIA (J00271, V00594, X97260, S52379, P02795)

Interferon gamma (AF330164)

Platelet-derived growth factor B chain (PDGFB) (Y14326, XM_{—}009997)

Platelet-derived growth factor alpha polypeptide (PDGFA) (NM_{—}002607)

Neuregulin 1 (NRG1) (NM_{—}013964)

Heregulin-beta1 (M94166)

TNF-alpha (AB048818)

TNF-beta (Lymphotoxin) (D12614)

Oxytocin receptor (OXTR) (NM_{—}000916, X80282 M25650)

Kappa light chain nuclear factor, NFKB (L01459)

Selectin P (NM_{—}003005)

Selectin E (NM_{—}000450)

Integrin, alpha (NM_{—}000885)

Hormone-sensitive lipase (NM_{—}005357)

TGF-beta 1 (A18277)

ICAM-1 (X84737)

GM-CSF (AJ224149)

CD8 antigen (NM_{—}004931)

CD11A antigen, integrin alpha L (XM_{—}008099)

CD11b (NM_{—}000632)

CD11C (NM_{—}000887)

CD28 glycoprotein (AH002636)

CD34 antigen (CD34) (NM_{—}001773)

CD40 (XM_{—}009624)

CD40 ligand (X67878 S50586)

CD44 (NT_{—}024229)

CD54 (NT_{—}011130 NT_{—}004939)

CD58 (XM_{—}001325)

CD62L (NT_{—}004939)

CD69 antigen (BC007037)

CD80 antigen (CD28 antigen ligand 1, B7-1 antigen) (XM_{—}002948)

CD86 antigen (CD28 antigen ligand 2, B7-2 antigen) (XM_{—}002802)

Interleukin 1, beta (IL1B) (NM_{—}000576)

Interleukin 1 receptor antagonist (IL1-RA) (XM_{—}010756 P18510 NM_{—}000577 AJ005835 BC009745 M55646 M63099 X52015 X53296 X64532 X84348 AF043143)

Interleukin 2 (IL2) (AF359939)

Interleukin 2 receptor, beta (IL2R) (XM_{—}009962)

Interleukin 4 (IL4) (AF395008)

Interleukin 5 (IL5) (AF353265)

Interleukin 6 (IL6) (AF048692)

Interleukin 10 (IL10) (XM_{—}001409)

Interleukin 12A (NM_{—}000882)

Interleukin 12B (NM_{—}002187)

Interleukin 13 (IL13) (AF377331)

Interleukin 16 (NM_{—}004513)

Aldose reductase (BC010391)

Neutrophil elastase (AC004799)

Folate binding protein (FBP) (X62753)

Cytochrome c oxidase subunit Vb (Cox Vb) (M19961)

Cytochrome c oxidse subunit IV (Cox IV) (BC008704)

Transcription factor A, mitochondrial (TFAM) (NM_{—}012251)

ATP synthase beta (NM_{—}001686)

Prolactin (PRL) (XM_{—}004269)

Retinoic acid receptor, beta (RARB) (XM_{—}003071)

Choline acetyltransferase (CHAT) (XM_{—}011848)

Cholinergic receptor, nicotinic, beta polypeptide 4 (CHRNB4) (NM_{—}000750)

RAF1 (NM_{—}002880)

Nicotinic acetylcholine receptor (AChR) (X17104)

Acetylcholine receptor delta subunit (X55019 X53091 X53516)

Cholinergic receptor, nicotinic, epsilon polypeptide (XM_{—}008520)

PKC alpha (X52479)

v-Ha-ras (XM_{—}006146)

v-fos FBJ murine osteosarcoma viral oncogene homolog (FOS) (NM_{—}005252)

Cytochrome P450 monoxygenase CYP2J2 (U37143)

Fibronectin (E01162)

Vascular cell adhesion molecule 1 (VCAM-1) (X53051)

PECAM1 (NM_{—}000442)

MCP-1 (Y18933)

AP-2 (X77343)

Apob-100 (M14162)

Actin, beta (ACTB) (XM_{—}004814)

GAPDH (NT_{—}009731)

Cyclin-dependent kinase 4 (CDK4) (NM_{—}000075)

Cyclin-dependent kinase 2 (CDK2) (XM_{—}006726)

Human cyclin D1 (M64349)

Human cyclin D2 (X68452)

Human cyclin A1 (NM 003914)

Skeletal muscle alpha-actin (ACTA1) (AF182035)

Retinoic acid receptor, alpha (BC008727)

Transforming growth factor-beta (TGF-beta) (X02812 J05114)

Beta-1-adrenergic receptor (ADRB1) (AF169007)

Adrenergic, beta-2-, receptor, surface (ADRB2) (NM_{—}000024)

Insulin (BC005255)

Leptin (Lep) (U65742)

Leptin receptor db form (OB-Rdb) (U58863)

Myelin basic protein (MBP) (XM_{—}008797)

RANTES (AF088219)

MIP-1 alpha/RANTES receptor (E13385)

MIP-1 beta (NT 010795)

Chemokine (C-C motif) receptor 5 (CCR5) (NM_{—}000579)

Thioredoxin (TXN) (XM_{—}015718)

Thrombopoietin (XM_{—}002815)

Polyomavirus (NC_{—}001515 NC_{—}001516)

JC virus (J02226 J02227 NC_{—}001699)

SV40 (J02400 J02402-3 J02406-10 J04139 M24874 M24914 M28728 V01380 NC_{—}001669)

BK virus (NC_{—}001538 V01108 J02038 strain dunlop V01109 J02039 strain MM J02038 K00058 V01108 strain dunlop M23122 strain AS)

Lymphotropic polyomavirus (K02562)

Human adenovirus type 2 (NC_{—}001405)

Human adenovirus 5 (NC_{—}001406 M73260 M29978)

Human adenovirus type 5 E1A enhancer (MI3156)

Human adenovirus 17 (NC_{—}002067 AF108105)

Human adenovirus 40 (L19443)

Human herpesvirus 1 (NC_{—}001806 X14112 D00317 D00374 S40593)

Human herpesvirus 2 (NC_{—}001798)

Human herpesvirus 3 (NC_{—}001348)

Human herpesvirus 4 (NC_{—}001345)

Human herpesvirus 5 (NC_{—}001347 X04650 D00328 D00327 X17403 (strain AD169) M17956 M21295 U33331 D63854 K01263 M60321 X03922 M1129 M18921)

Human herpesvirus 6 (NC_{—}001664 X83413 (U1102, variant A) AB021506 (variant B, strain HST))

Human herpesvirus 6B (NC_{—}000898 AF157706 L13162 L14772 L16947 (strain Z29))

Human herpesvirus 7 (NC_{—}001716 U43400 (JI) AF037218 (strain RK))

Epstein-Barr virus (EBV) (V01555 J02070 K01729-30 V01554 X00498-99 X00784 (strain B95-8) L07923 X58140 D10059)

Rous sarcoma virus (NC_{—}001407)

Y73 sarcoma virus (NC_{—}001404)

Human coxsackievirus A (NC_{—}001429)

Coxsackievirus B3 (NC_{—}001473)

Moloney murine leukemia virus (NC_{—}001501 J02255 J02256 J02257 M76668 AF033811)

Human immunodeficiency virus type 1 (AJ006022 NC_{—}001802 K02013 K03455 M38432 AF286239 U86780 AF256211 AF256205 AF256207 AF256206 X04415 K03456)

Human immunodeficiency virus type 2 (NC_{—}001722 J04542 U27200 L14545 D00835 U38293 X05291 M31113 X52223 M15390 J04498 M30502 U22047 L07625 M30895 D00477 X61240 X16109 AF082339)

Human T-cell lymphotropic virus type 1 (AF033817 NC_{—}001436 AF259264 U19949 AF042071 J02029 M33896 AF139170 L03561)

Human T-cell lymphotropic virus type 2 (AF326584 NC_{—}001488 AF326583 AF139382 Y13051 Y14365 AF074965 NC_{—}001877) LCMV (Y16308 M20869 M22138 AF079517AF186080 AJ233196 AJ297484 AJ233200 AJ233161 AH004719 AH004717 AH004715 S75753 S75741 S75739 912860 912868)

TMEV (NC_{—}001366 AF030574 M80890 M80889 M80888 M80887 M80886 M80885 M80884 M80883 M16020 M14703 M20562 M20301 M94868)

Hepatitis B virus (NC_{—}001707 AF330110 AB042283 AB042282 AB050018 AB042284 AB049609 AB049610 AF182803 AB042285 AF182804 AF182805 AF182802 AF384371 AF363961 AF384372)

Collagen type I alpha2 (COL1A2) (M35391 K02568 AF004877 AC002528 M22817 M20904 XM_{—}004658 Z74616 L47668 NM 000089 M22816 M20904 J03464 M18057 X02488 M21671 Y00724 V00503 S89896 M64229 S96821 AB004317 L00613 U79752 S62614 S59218 S59211 S89898 X67667 P08123)

Collagen type I alpha 1 (COL1A1) (XM_{—}037910 AF017178)

Tissue factor (XM_{—}001322 J02931 J02681 NM_{—}001993 M16553 J02846 M27436 AL138758 A19048 P13726 P30931 AAB20755 KFBO3 X53521 KFRB3 P24055 AAA63469 CAA37597 AAF36523 Q9JLU8 M26071 AAA40414 KFMS3 NP_{—}034301 P20352 AAA63400 AAA16966 P42533 NP_{—}037189)

Integrin, beta 2 (CD18) (X64074 X63835 X64075 X63835 X64076 X63835 X64077 X63835 X64078 X63835 X64079 X63835 X64080 X63835 X64081 X63835 X64082 X63835 X64083 X63835 X63924 X63835 X63925 X63835 X63926 X63835 X64073 X63835 AL163300 AP001755 BA000005 BC005861 S81234 Y00057 M19545 M15395 NM_{—}000211 X64071 X63835 X63926 X63835 AH003850 S81231 S81252 S81247 S75381 S75297 M95293 M38701 X54481 M77675 P05107)

Rb1 (L11910 M27845 M27846 M27847 M27848 M27849 M27850 M27851 L35146 M27852 M27853 M27854 M27855 M27856 M27857 M27858 M27859 M27860 L35147 M27862 M27863 M27864 M27865 M27866 X16439 L41890 L41891 L41893 L41894 L41895 L41896 L41897 L41898 L41899 L41997 L41999 L41907 L41914 L41904 L41921 L41996 L41998 L42000 L41911 L41924 L41923 L41920 L41918 L41870 L49209 L49212 L49213 L49218 L49220 L49223 L49230 L49231 L49232 AH006304 AH005289 AH005290 AH005288 M26460 M28736 M15400 M28419 M33647 J02994 NM_{—}000321 AF043224 XM_{—}007211 M19701 J03809 AAA53483)

BRCA1 (U37574 XM_{—}008213 XM_{—}008214 XM_{—}008215 XM_{—}008216 XM_{—}008217 XM_{—}008219 XM_{—}008220 XM_{—}008221 XM_{—}008222 XM_{—}017568XM 017569XM 017570NM_{—}007294NM_{—}007295 NM_{—}007296 NM_{—}007297 NM_{—}007298 NM_{—}007299 NM_{—}007300 NM_{—}007301 NM_{—}007302 NM_{—}007303 NM_{—}007304 NM_{—}007305 NM_{—}007306 U14680 AF005068 U68041 U64805 Y08864 XP_{—}017569 XP_{—}008212)

Fas (X63717 NM_{—}000043 X83493 X89101 Z47993 Z47994 Z47995 Z70519 Z70520 P25445)

p300 (XM_{—}010013 U01877 NM_{—}001429 Q09472 S67605 AL096765)

CREB-binding protein (CBP) (AC004760 NP_{—}004371 AJ251844 U47741 U85962 U89354 U89355 XM_{—}036668 XM_{—}036667 XM_{—}036669 BG710081 S66385 U88570)

ZF_TAZ matrix, p300/cbp protein binding site (PS50134 XM_{—}017011 XM_{—}009709XM_{—}017011 AF078104 M74515 M74511 AF057717)

E4TF1-60 (D13318 X84366)

E4TF1-53 (D13317)

E4TF 1-47 (D 13316)

Human nuclear respiratory factor-2 subunit alpha (U13044)

Human nuclear respiratory factor-2 subunit beta 1 (U13045)

Human nuclear respiratory factor-2 subunit beta 2 (U13046)

Human nuclear respiratory factor-2 subunit gamma 2 (U13048)

GA-binding protein, subunit beta 1 (NM_{—}005254 NM_{—}016654 BC004103 M74516 M74512)

GA-binding protein, subunit beta 2 (NM_{—}002041 NM_{—}016655 M74517 M74513)

GA-binding protein, subunit gamma 1 (U13047)

Ets1 (J04101 X14798NM_{—}005238 M11921 XM_{—}015368 XP_{—}015368)

ERK1 (AJ222708 NM_{—}002745 M84490 BC000205 Z11696 S38872 P27361 Z11694 S38867 Z11695 S38869)

ERK2 (M84489 P28482)

JNK1 beta 2 (U35005)

JNK1 beta 1 (U35004)

JNK2 beta 2 (U35003)

JNK2 beta 1 (U35002)

JNK1 alpha 2 (U34822)

JNK2 alpha 1 (U34821)

JNK3 alpha 1 (U34820)

JNK3 alpha 2 (U34819)

JNK2 (L31951)

JNK1 beta 2 (AAC50611)

MEK1 (L05624NM_{—}002755 Q02750)

MEK kinase 1 (MEKK1) (AF042838)

MEK kinase 3 (MEKK3) (U78876)

Human STAT1 (P42224 NM_{—}007315 AF182311 BC002704 M97936 U18662 U18663 U18664 U18665 U18666 U18667 U18668 U18669 U18670) Human STAT2 (U18671 M97934 S81491 P52630)

Human IL-2 receptor, gamma (NM_{—}000206 D11086 L12183 AC087668 L19546 P31785)

Alpha 2 adrenergic receptor (M18415)

Beta 3 adrenergic receptor (P13945 X72861)

Beta 3 adrenergic receptor X70811)

Beta 3 adrenergic receptor (X70812)

Beta 3 adrenergic receptor (S53291)

CCAAT/enhancer binding protein (C/EBP) (NM_{—}005194)

Cbp/p300-interacting transactivator (BC004240)

AML1 (AF312387 AF025841 AF312386 AY004251) AML (D10570)

AML1 (D43967 D43969 D89788 D89789 D89790 L21756 L34598 M83215 U19601 X79549 X90976 X90978 X90981 AP001721 Q0196)

A-Myb (X66087 S75881 X13294 P10243)

ATF1 (X55544)

ATF2 (P15336 AY029364 M31630 U16028 X15875)

ATF4 (P18848 AL022312 BC008090 BC011994 D90209 M86842)

c-Fos (P01100 AB022276 AF111167 BC004490 K00650 V01512)

AP1 (P05412 AL136985 BC002646 BC006175 BC009874 J04111)

C2TA (P33076 AF410154 U18259 U18288 U31931 X74301)

c-Myb (P10242 AF104863 M13665 M13666 M15024 U22376 X52125 P17676 AL161937 BC005132 BC007538 X52560 P16220 BC010636 M27691 M34356 S72459 X555450

CREB (X60003 O431860

CRX (AF024711)

CID (P19538)

DBP (Q10586 BC011965 D28468 U06936 U48213 U792830

E2F1 (Q01094 AF086380 AL121906 BC005098 M96577 S49592 S74230 U47675 U47677)

E2F2 (Q14209 AL021154 L22846)

E2F3 (000716 AL136303 D38550 Y10479)

Egr1 (P18146 AJ243425 M62829 M80583 X52541)

ELK1 (P19419 AB016193 AB016194 AF000672 AF080615 AF080616 AL009172 M25269)

Ets2 (P15036 AF017257 AL163278 AP001732 J04102 M11922 X55181)

ER81 (P50549 AC004857 U17163 X87175 P03372 AF120105 AF172068 AF172069 AF258449 AF258450 AF258451 AL078582 AL356311 M112674 S80316 U476780

ER alpha (X03635 X624620

ER beta (Q92731 AB006589 AB006590 AF051427 AF051428 AF060555 AF061054 AF061055 AF074598 AF074599 AF124790 AF215937 X99101)

GATA1 (P15976 AF196971 BC009797 M30601 X17254)

Gli3 (P10071 AC005028 AJ250408 M20674 M57609 P04150 AC005601 BC015610 M109010

GR (M69104 M73816 U01351 U80946 X03225 X03348 Q16665 AF050127 AF207601 AF207602 AF2084870

HIF1A (AF304431 BC012527 U22431 U29165 U85044 X72726)

HNF4A (P41235 AL132772 U72967 X76930 X87870 X87871 X87872 Z49825)

JunB (P17275 BC004250 BC009465 BC009466 M29039 U20734 X51345)

MDM2 (Q00987 AF201370 AF385322 AF385323 AF385324 AF385326 AF385327 AJ276888 AJ278975 AJ278976 AJ278977 AJ278978 BC009893 M92424 U33199 U33200 U33201 U33202 U33203 Z12020 NM_{—}006878 NM_{—}006879 NM_{—}006880 NM_{—}006881 NM_{—}006882)

MDMD2 (AF385325)

MEF2C (Q06413 L08895 S57212)

Mi (075030 AB006909 AB009608 AB032357 AB032358 AB032359 AL110195 Z29678)

MyoD (P15172 AF027148 BC000353 X17650 X56677)

RelA (Q04206 BC011603 BC014095 LI 9067 M62399 Z22948 Z22951)

NFAT1 (Q13469 AL035682 U43341 U43342)

NF-YB (P25208 BC005316 BC005317 BC007035 L06145 X59710)

NF-YA (P23511 NM_{—}021705 AK025201 AL031778 M59079 X59711)

P/CAF (Q92831)

p/CIP (Q9Y6Q9 AL0344180 Q9UPG4)

MRG1 (Q99967 AF109161 AF129290 BC004377 U65093)

NFE2 (Q16621 BC005044 L13974 L24122 S77763 P04637 AF052180 AF066082 AF135121 AF136271 AF307851 BC003596 K03199 M13121 M14694 M14695 M22881 M22898 U94788 X01405 X02469 X541560 X60010 X600110

p53 (X60012 X60013 X60014 X60015 X60016 X60017 X60018 X60019 X60020)

p73 (015350 AF077628 AL136528 Y11416)

RSK1 (NM_{—}002953 AL109743 BC014966 L07597 Q15418)

RSK3 (AL022069 AX019387 BC002363 L07598 X85106)

RSK2 (P51812 L07599 U08316)

PIT1 (P28069 D10216 D12892 L18781 X62429 X72215)

RARG (P13631 AJ250835 L12060 M24857 M38258 M57707 P22932)

RXRA (AF052092 BC007925 BC009882 U66306 X52773 Q08211 L13848 U03643 Y10658 P28324 NM_{—}001973 M85164 M85165 Q13285 D842060

SF-1 (D84207 D84208 D842090 D84210 D88155 U76388 Q13485 AF0454470 SMAD4 (BC002379 U44378 Q15797 BC001878 U548260

SMAD1 (U57456 U59423 U59912)

SMAD2 (Q15796 AF027964 BC014840 U59911 U65019 U68018 U78733)

SMAD3 (Q92940 U68019 U76622)

SRC1 (AJ000882 NM_{—}0037430 AJ000881 U19177 U19179 U40396 U59302 U90661)

SREBP1 (P36956 U00968)

SREBP2 (Q12772 U02031 Z99716)

STAT3 (P40763 AJ012463 BC000627 BC014482 L29277)

STAT4 (Q14765)

STAT5A (P42229 L41142 U43185)

STAT5B (P51692 U47686 U48730 P42226 AF067572 AF067573 AF067574 AF067575 BC004973 BC0058230

STAT6 (U16031 U66574)

TAL1 (P17542 AJ131016 AL135960 M29038 M61108 S53245 X51990)

TBP (P20226 AL031259 M34960 M55654 X54993)

TF2B (Q00403 AL445991 S44184)

THRA (P10827 BC000261 BC002728 J03239 M24748 M24899 X55005 X55074 Y00479)

THRB (P10828 M26747 X04707 P37243)

TWIST (Q15672 U80998 X91662 X99268 Y10871)

IRF3 (Q14653 AF112181 AX015330 AX015339 BC009395 U86636 Z56281)

YY1 (P25490 AF047455 M76541 M77698 Z14077)

PPARG (P37231 NM_{—}015869 BC006811 D83233 L40904 U63415 U79012 X90563)

AR (P10275 AF162704 L29496 M20132 M20260 M21748 M23263 M27430 M34233 M35851 M58158 S79366 S79368 M27424 M27425 M27426 M27427 M27428 M27429 M35845 M35846 M35847 M35848 M35849 M35850)

SRD5A1 (P18405 AF052126 AF113128 AL008713 BC006373 BC007033 BC008673 M32313 M68886 M68882 M68883 M68884 M68885 AF073302 AF073304)

(b) Equivalent Molecules

The term “equivalent molecules” is understood to include molecules having the same or similar activity as the molecule of interest, including, but not limited to, biological activity and chemical activity, in vitro or in vivo.

(c) Homologous Molecules

The term “homologous molecules” is understood to include molecules with the same or similar chemical structure as the molecule of interest (see exemplary embodiments above).

The following section presents standard assays, which can be used, in conjunction with the assays in the new elements section, to test the effect of an agent on a molecule of interest.

(d) During

The term “during drug discovery, development, use as treatment, or during diagnosis” is understood to include, but not be limited to, drug screening, rational design, optimization, in laboratory or clinical trials, in vitro or in vivo (see exemplary embodiment below).

(2) Assaying Protein Concentration

(a) UV Absorbance

In one exemplary embodiment, cellular protein concentration is measured by virtue of its absorbance of ultraviolet light at the wavelength of 280 nm (Ausubel 1999¹⁴⁸). To calibrate the reagents used, and to validate the spectrophotometer, a standard curve is established using protein solutions of known concentration. Typically solutions of bovine serum albumin, a commonly available protein, are used to establish the standard curve. Cells are lysed in a detergent-rich buffer to liberate membrane associated and intracellular proteins. Following lysis, insoluble materials are removed by centrifugation. The absorbance of UV light by the supernatant, which contains soluble proteins of unknown concentration, is then measured and compared to the standard curve. Comparison of the data obtained from the cellular extracts with those represented by the standard curve provides an indication of cellular protein concentration.

(b) Bradford Method

In another exemplary embodiment, protein concentration is determined using the Bradford method (Sapan 1999¹⁴⁹, Ausubel 1999, Ibid). A standard curve is constructed using solutions of known protein concentration mixed with coomassie brilliant blue. Following a brief incubation at room temperature, the absorbance of light at 595 nm is measured and a standard curve is constructed. Cells are lysed as described above, the lysate is mixed with coomassie brilliant blue and the absorbance measured in a manner identical to that of the standard curve. Comparison of the values obtained from the cellular extract with those of the solutions of known concentration reveals the concentration of cellular proteins.

(c) Immunoaffinity Chromatography

To measure concentration of a specific cellular protein, for instance, p300, GABP or CBP, additional steps are employed to purify the protein away from other cellular proteins. One exemplary embodiment involves the use of specific antibodies targeted against the protein of interest to remove it from the cellular lysate. Specific antibodies, for instance, anti-p300, anti-GABP or anti-CBP, are chemically bound to a resin and contained within a vertical glass or plastic column. Cell lysate is passed over that resin to permit antibody-antigen interactions, thereby allowing the protein to bind to the immobilized antibodies. Efficient removal of the protein of interest from the cell lysate is accomplished by using an excess of antibody. Protein bound to the column is removed which releases the bound protein. The eluted protein is collected and its concentration determined by an assay for protein concentration such as those exemplified above.

(3) Assaying mRNA Concentration

(a) UV Absorbance

In certain embodiments, RNA concentration is measured by absorption of ultraviolet light at a wavelength of 260 nm (Manchester 1995¹⁵⁰, Davis 1986¹⁵¹, Ausubel 1999, Ibid). RNA is purified from cells by first lysing the cells in a detergent rich buffer. Proteins in the cellular lysate are degraded by incubation overnight at 65° C. with proteinase K. After enzymatic degradation, proteins are extracted from the solution by mixing with phenol/chloroform/isoamyl alcohol followed by extraction with chloroform/isoamyl alcohol. Nucleic acids in the resulting protein deficient solution are precipitated by addition of salt, typically sodium acetate or ammonium acetate, and ethanol. After a brief incubation of the mixture at −20° C., the insoluble nucleic acids are removed by centrifugation, dried, and redissolved in a sterile, RNase free solution of Tris and EDTA. Contaminating DNA is removed from the lysate by treatment with RNase-free DNase I. Degraded DNA is removed by precipitation of the intact RNA with salt and ethanol. The dried, purified RNA is dissolved in Tris-EDTA and quantified by virtue of its absorbance of light at 260 nm. Since the molar extinction coefficient of RNA at 260 nm is well known, the concentration of RNA in the solution can be determined directly.

(b) Northern Blot

The concentration of a particular RNA species can also be determined. In one exemplary embodiment, the amount of mRNA which encodes a protein of interest, for instance, p300, GABP, CBP, within a population of cells is measured by Northern blot analysis (Ausubel 1999, Ibid, Gizard 2001¹⁵²). Total cellular RNA is isolated and separated by electrophoresis through agarose under denaturing conditions, typically in a gel containing formaldehyde. The RNA is then transferred to, and immobilized upon a charged nylon membrane. The membrane is incubated with a solution of detergent and excess of low molecular weight DNA, typically isolated from salmon sperm, to prevent adventitious binding of the gene specific, for instance, p300-, GABP-, CBP-specific, radiolabeled DNA probe to the membrane. Radiolabeled cDNA probes representing the protein, e.g., p300, GABP, CBP, are then hybridized to the membranes and bound probe is visualized by autoradiography.

(c) Reverse Transcriptase—Polymerase Chain Reaction (RT-PCR)

In another exemplary embodiment, the amount of mRNA encoding a protein of interest, for instance, p300, GABP, CBP, expressed by a population of cells is measured by first isolating RNA from cells and preparing cDNA by binding oligo deoxythymidine (dT) to the polyadenylated mRNA within the prepared RNA. Reverse transcriptase is then used to extend the bound oligo dT primers in the presence of all four deoxynucleotides to create DNA copies of the mRNA. The cDNA population is then amplified by the polymerase chain reaction in the presence of oligonucleotide primers specific for the sequence of the gene or RNA of interest and Taq DNA polymerase. The amplification products can be visualized by gel electrophoresis followed by staining with ethidium bromide and exposure to ultraviolet light. Quantification can be achieved by adding a radiolabeled deoxynucleotide to the PCR reaction. Radiolabel incorporated into the amplification products is visualized by autoradiography and quantified by densitometric analysis of the autoradiograph or by direct phosphorimager analysis of the electrophoretic gel.

(d) S1 Nuclease Protection

In a related exemplary embodiment, expression of RNA encoding a protein of interest, for instance, p300, GABP, CBP, can be assessed by hybridizing isolated cellular RNA with a radiolableled synthetic DNA sequence homologous to the 5′ terminus of the RNA of the protein of interest. The synthetic deoxyribonucleotide, less than 40 nucleotides in length, is labeled at it 5′ end with T4 polynucleotide kinase and γ-³²P ATP. Once the oligonucleotide is bound to the RNA, the mixture is incubated in the presence of the single strand-specific nuclease S1. Any unhybridized, and therefore single stranded, molecules of RNA or DNA are degraded, leaving the DNA-RNA hybrids of the protein of interest intact. The undegraded hybrids are removed from the solution by precipitation with ammonium acetate and ethanol and resolved by nondenaturing gel electrophoresis.

Radiolabeled bands on the gel are then visualized by autoradiography. The radiolabel can be quantified by densitometric analysis of the autoradiographs or by phosphorimager analysis of the electrophoretic gels themselves.

(4) Assaying Polynucleotide Copy Number

(a) S1 Nuclease Protection

This same technique can be used to quantify the level of any nucleic acid, naturally expressed or exogenous, within a population of cells. In every case the sequence of the single stranded synthetic oligonucleotide must be designed so that it is complementary to the 5′ terminal sequence of the species to be measured.

(b) Real Time PCR

In another exemplary embodiment, DNA copy number can be measured using real time PCR (Heid 1996¹⁵³). This technique employs oligonucleotides doubly labeled. At the 5′ ends they carry a reporter dye that fluoresces upon excitation by the appropriate wavelength of light. At the 3′ end they carry a quencher dye that suppresses the fluorescence of the first dye. These oligonucleotides are prepared so that their sequence is complementary to the region of interest, which lies between the forward and reverse PCR primers. Once hybridized to the DNA sequence of interest, the close proximity of the quencher dye and the fluorescent dye suppresses the fluorescent emissions of the reporter dye. However, during the process of PCR, Taq polymerase cleaves the reporter dye from the oligonucleotide and releases it. Once removed from the nearby quencher dye, fluorescence is permitted. Free fluorescent dye is quantified with a fluorimeter and is directly related to the number of molecules of interest present prior to PCR.

(5) Detection of Binding

(a) General

In one exemplary embodiment, an assay to identify compounds that bind to a polynucleotide or polypeptide of interest involves binding of a test compound to wells of a microtiter plate by covalent or non-covalent binding. For instance, the assay may anchor a specific test compound to a microtiter plate substrate using a mono or polyclonal immobilized antibody. A solution of the test compound can also be used to coat the solid surface. Then, the nonimmobilized polynucleotide or polypeptide of interest may be added to the surface coated wells. After sufficient time is allowed for the reaction to complete, the residual components are removed by, for instance, washing. Care should be taken not to remove complexes anchored on the solid surface. Anchored complexes may be detected by several methods known in the art. For instance, if the nonimmobilized polynucleotide or polypeptide of interest, or test compound were labeled before the reaction, the label may be used to detect the anchored complexes. If the components were not prelabeled, a label may be added during or after complex formation, for instance, an antibody directed against the nonimmobilized polynucleotide or polypeptide of interest, or test compound, can be added to the surface coated wells.

In a variation of this assay, the polynucleotide or polypeptide of interest is anchored to a solid surface and the nonimmobilized test compound is added to the surface coated wells.

In another variation of this assay, the reactions are performed in a liquid phase, and the complexes are removed from the reaction mixture by immunoaffinity chromatography, or immunoprecipitation, as described herein.

(b) Detection of Binding to DNA

In one exemplary embodiment, DNA fragments carrying a known, or suspected binding domain for a polypeptide of interest, for instance, p300, GABP, etc., are purified by gel electrophoresis and labeled with T4 polynucleotide kinase in the presence of γ³²P-ATP (Bulman et al. 2001). Labeled DNA is then added to a solution containing the polypeptide of interest under conditions, ionic and thermal, which permit formation of DNA-polypeptide complexes. The solution is then maintained for a period of time sufficient for the reaction to complete. Following completion, the mixture is separated by electrophoresis through nondenaturing polyacrylamide in parallel to labeled, but otherwise unreacted test DNA. Following electrophoresis, the labeled DNA is detected by autoradiography or by phosphorimager analysis. Formation of complexes is detected by the shift in electrophoretic mobility (see also below).

The assay detects polypeptide-DNA complexes formed by direct binding of the polypeptide of interest with DNA, or by indirect binding through intermediary polypeptides, as long as the intermediary polypeptides are present in the reaction mixture. Further, the magnitude of the gel shift provides a semi-quantitative measure of the relative concentration of the polypeptide-DNA binding in the assay mixture. As such, changes in concentration can also be detected.

(i) Affinity Chromatography

In one exemplary embodiment, binding of a polypeptide of interest, that is, disrupted polypeptide, or polypeptide in a disrupted or disruptive pathway, such as p300, GABP, CBP, to DNA is measured by first expressing fragments of the polypeptide of interest as GST (glutathione sulfonyl transferase) fusion proteins in E. coli (Gizard 2001, Ibid). The expressed polypeptides are then bound to glutathione coupled sepharose. Radiolabeled DNA fragments, carrying ³²P, representing the polypeptide binding site, are incubated with protein-bead complexes and subsequently washed three times to remove adventitiously bound DNA. Any DNA bound to the immobilized polypeptide of interest is released by boiling in presence of the ionic detergent SDS. Liberated radiolabeled DNA is quantified by liquid scintillation counting, or by direct measurement of Cerenkov radiation.

(ii) Electrophoretic Gel Mobility Shift Assay

In another exemplary embodiment, binding of a polypeptide of interest, or a group of polypeptides to DNA is assessed by electrophoretic gel mobility shift assay (Gizard 2001, Ibid, Ausubel 1999, Ibid, Nuchprayoon 1999¹⁵⁴). Radiolabeled DNA carrying the polypeptide binding site, for instance, the p300 binding site, or N-box, is mixed with the recombinant polypeptide, for instance, p300, GABP, expressed as GST fusion protein. Subsequent resolution by electrophoresis through nondenaturing polyacrylamide gels in parallel with labeled DNA alone, reveals a shift in electrophoretic mobility only if the polypeptide is bound to DNA in the DNA/polypeptide mixtures. If the DNA binding site is unknown, or one is suspected to be carried in a collection of DNA fragments, this assay can be performed to test for, and potentially affirm the presence of such a binding site.

(6) Detection of Binding Interference

A polynucleotide or polypeptide of interest may bind with one or many cellular or extracellular proteins in vivo. Compounds that interfere with, or disrupt the binding may include, but are not limited to, antisense oligonucleotides, antibodies, peptides, and similar molecules.

In one exemplary embodiment, binding interference of a test compound is assessed by adding the compound to a mixture containing a polynucleotide or polypeptide of interest and a binding partner. After enough time is allowed for the reaction to be completed, the complex concentration in the test reaction mixture is compared to a control mixture prepared without the test compound, or with a placebo. A decreased concentration in the test reaction indicates interference. Reactants may be added at different orders regardless of the method used. For example, a test compound may be added to the reaction mixture before adding the polynucleotide or polypeptide of interest and their binding partners, or at the same time. A test compound that can disrupt an already formed complex, for instance, by displacing a complex component, can be added to the reaction mixture after complex formation. The interference assay can be conducted in two ways, in liquid, or in solid phases, as described above.

In another embodiment, a polynucleotide or polypeptide of interest is prepared for immobilization by fusion to glutathione-S-transferase (GST), while maintaining the binding capacity of the fusion protein. Another complex component, a cellular polynucleotide or polypeptide, or extracellular protein, can be purified, and then utilized in developing a monoclonal antibody using methods well known in the art. The GST-polynucleotide fusion protein is coupled to glutathione-agarose beads and exposed to the other complex component in the presence or absence of a test compound. After sufficient time has been allowed for the reaction to complete, unbound components are removed, for instance, by washing, and the labeled monoclonal antibody is added. Bound radiolabeled antibody is then measured to quantify the extent of complex formation. Inhibition of complex formation by a test compound decreases measured radioactivity. As above, a test compound capable of complex disruption can also be added after complex formation.

In one variation of the assay, the fusion protein is mixed with the other complex component in liquid, that is, without solid glutathione-agarose beads.

In another variation of the assay, peptide fragments of the binding domains, instead of full-length complex components are used. Several methods well known in the art can be used to identify and isolate binding domains. For instance, one method entails mutating a gene and screening for a disruption in normal binding of the polypeptide encoded by the gene by co-immunoprecipitation or immunoaffinity. If the polypeptide shows disrupted binding, analysis of the gene sequence can reveal the binding domain, or the region of the polypeptide involved in binding. Another approach partially proteolyzes a labeled polypeptide anchored to a solid surface. Non-bound fragments are removed by washing leaving a labeled polypeptide comprising the binding domain immobilized on the solid surface. The polypeptide fragments bound to the immobilized proteins are than isolated and analyzed by amino acid sequencing, using for instance the Edman degradation procedure (Creighton 1983¹⁵⁵). Another approach expresses specific fragments of a polynucleotide, or gene, and tests the fragments for binding activity.

In another embodiment, an assay uses a complex with one component labeled. However, binding to the complex quenches the signal generated by the label (see, for instance, U.S. Pat. No. 4,109,496). A test compound which disrupts the complex, for instance, by displacing a part of the complex, restores the signal. This assay can be used to identify compounds which either interfere with complex formation, or disrupt an already formed complex.

Specifically, a test compound can interfere with binding between a disrupted gene or polypeptide, or a gene or polypeptide in a disruptive or disrupted pathway, for instance, a microcompeted or mutated gene or polypeptide, and their binding partner. The assay may be especially useful in identifying compounds capable of interfering in binding reactions between foreign polynucleotides and cellular polypeptides without interfering in binding between cellular polynucleotide and cellular polypeptides. The assay is also especially useful in identifying compounds capable of interfering in binding between mutant cellular polynucleotide, or polypeptide, and normal cellular polynucleotide, or polypeptide, without interfering in binding between normal polynucleotide or polypeptides.

(7) Identification of a Polypeptide Bound to DNA or Protein Complex

(a) Immunoprecipitation

In one exemplary embodiment, the identity of a bound polypeptide, for instance, p300, GABP, CBP, is confirmed by reacting antibodies specific to the polypeptide of interest with polypeptides bound to DNA. For example, p300-specific antibodies are mixed with the polypeptide-DNA complexes and incubated overnight at 4° C. Immune complexes are then precipitated by the addition of a secondary antibody directed against the primary p300-specific antibody. Precipitated antibody-antigen complexes are resolved by denaturing gel electrophoresis and the constituent proteins are visualized by staining with coomassie brilliant blue.

In a related exemplary embodiment, the interaction between a polypeptide of interest, for instance, p300, GABP, CBP, and other cellular proteins, such as transcription factors, may be detected by co-immunoprecipitation of the polypeptide of interest with antibodies specific to the polypeptide, for instance, p300-specific antibodies. For example, in the case of p300, cellular protein extracts are incubated with purified p300-GST fusion proteins to enable protein-protein interactions. p300-specific antibodies are then added and the mixture is incubated overnight at 4° C. Immune complexes are precipitated by addition of a secondary antibody directed against the primary p300 antibodies and the precipitates are resolved by electrophoresis on denaturing polyacrylamide gels. Proteins are subsequently detected by staining with coomassie brilliant blue.

(b) Antibody Supershift Assay

In a related exemplary embodiment, DNA-protein complexes are detected by electrophoretic gel mobility shift assay (Gizard 2001, Ibid, Ausubel 1999, Ibid). Radiolabeled DNA carrying the polypeptide binding site, for instance, p300 binding site, or N-box, is mixed with a recombinant polypeptide, for instance, p300, or GABP, expressed as GST fusion protein. Subsequent resolution by electrophoresis through nondenaturing polyacrylamide gels in parallel with labeled DNA alone, reveals a shift in electrophoretic mobility only if the polypeptide is bound to DNA in the DNA/polypeptide mixture. To identify the bound polypeptide, a specific antibody is reacted to the DNA/polypeptide mixture prior to electrophoresis. Bound antibody molecules cause a further change in gel mobility, namely a supershift, and serve to identify the polypeptide bound to DNA.

(8) Identification of a DNA Consensus Binding Site

(a) PCR and DNA Sequencing

In one exemplary embodiment, DNA fragments are prepared containing potential polypeptide binding sites, either wild-type or variants, flanked by DNA fragments of known nucleotide sequence. The fragments are then reacted with the polypeptide-GST fusion proteins immobilized on sepharose beads. After washing to remove adventitiously bound DNA, bound fragments are eluted by heating in presence of a detergent. The eluted fragments are amplified by the polymerase chain reaction (PCR) using primers specific for the flanking DNA sequences. The nucleotide sequence of the amplification products is then determined by any sequencing method known in the art, for instance, the dideoxy chain termination sequencing method of Sanger (Sanger 1977¹⁵⁶), using as sequencing primer one of the two PCR primers. Several sequence variants of the binding site are likely to be identified. Together they can be used to establish a consensus DNA sequence for the polypeptide binding site.

(9) Detection of a Genetic Lesion

Existence of a genetic lesion can be determined by observing one or more of the following irregularities.

1. Deletion of at least one nucleotide from a disrupted gene, or gene in a disrupted pathway.

2. Addition of at least one nucleotide to a disrupted gene, or a gene in a disrupted pathway.

3. Substitution of at least one nucleotide to a disrupted gene, or gene in a disrupted pathway.

4. Irregular modification of a disrupted gene, or gene in a disrupted pathway, such as change in DNA methylation patterns.

5. Gross chromosomal rearrangement of a disrupted gene, or gene in a disrupted pathway, for instance, translocation.

6. Allelic loss of disrupted gene, or gene in a disrupted pathway.

7. Different than wild-type mRNA concentration of a disrupted gene, or gene in a disrupted pathway.

8. Irregular splicing pattern of mRNA transcript of a disrupted gene, or gene in a disrupted pathway.

9. Irregular post-transcriptional modification of an mRNA transcript other than splicing, for instance, editing, capping or polyadenylation, of a disrupted gene or gene in a disrupted pathway.

10. Different than wild-type concentration of a disrupted polypeptide, or polypeptide in a disrupted pathway.

11. Irregular post-translational modification of a disrupted polypeptide, or a polypeptide in a disrupted pathway.

Many assays are known in the art for detection of the above, or other irregularities associated with a genetic lesion. Consider the following exemplary assays. Also consider the exemplary assays discussed in the following reviews on detection of genetic lesions, Kristensen 2001¹⁵⁷, Tawata 2000¹⁵⁸, Pecheniuk 2000¹⁵⁹, Cotton 1993¹⁶⁰, Prosser 1993¹⁶¹, Abrams 1990¹⁶², Forrest 1990¹⁶³.

(a) Sequencing

In one exemplary embodiment, a polynucleotide of interest can be sequenced using any sequencing techniques known in the art to reveal a lesion by comparing the test sequence to wild-type control, known mutant sequence, or sequences available in public databases.

An introduction to sequencing is available in Graham 2001¹⁶⁴. Exemplary sequencing protocols are available in Rapley 1996¹⁶⁵. Recent sequencing methods are available in Marziali 2001¹⁶⁶, Dovichi 2001¹⁶⁷, Huang 1999¹⁶⁸, Schmalzing 1999¹⁶⁹, Murray 1996¹⁷⁰, Cohen 1996¹⁷¹; Griffin 1993¹⁷². Automated sequencing methods are available in Watts 2001¹⁷³, MacBeath 2001¹⁷⁴, and Smith 1996¹⁷⁵. For classical sequencing methods see Maxam 1977¹⁷⁶, Sanger 1977 (Ibid).

(b) Restriction Enzyme Cleavage Patterns

In another exemplary embodiment, patterns of restriction enzyme cleavage are analyzed to reveal lesions in a polynucleotide of interest. For example, sample and control DNA are isolated, amplified, if necessary, digested with one or several restriction endonucleases, and the fragments separated by gel electrophoresis. Sequence specific ribozymes are then used to detect specific mutations by development or loss of a ribozyme cleavage site.

(c) Protection from Cleavage Agents

In another exemplary embodiment, cleavage agents, such as certain single-strand specific nucleases, hydroxylamine, osmium tetroxide or piperidine, are used to detect mismatched base pairs in nucleic acid hybrids comprised of either RNA/RNA or RNA/DNA duplexes. Wild-type and test DNA or RNA, with one or the other molecule labeled with radioactivity, are mixed under conditions permitting formation of heteroduplexes between the two species. Following hybridization, the duplexes formed are treated with an agent capable of cleaving single, but not double stranded nucleic acids. Examples include, but are not limited to S1 nuclease, piperidine, hydroxylamine and RNase H, in the case of RNA/DNA heteroduplexes. Since mismatches between wild-type and mutant oligonucleotide result in single stranded regions, mismatch sites are susceptible to digestion. Once cleaved, the nucleic acid fragments are separated according to size by native polyacrylamide gel electrophoresis. Genetic lesion are detected by, for instance, observing different fragment sizes in test relative to wild-type DNA or RNA.

Examples of such assay in practice are available in Saleeba 1992¹⁷⁷, Takahashi 1990¹⁷⁸, Cotton 1988¹⁷⁹, Myers 1985¹⁸⁰, Myers 1985¹⁸¹.

(d) Mismatched Base Pairs Recognition

In another exemplary embodiment, mismatch cleavage reactions are carried out using one or more proteins capable of recognizing mismatched base pairs. The proteins are typically components of the naturally occurring DNA mismatch repair mechanism. In a preferred embodiment, the mutY enzyme derived from E. coli cleaves the adenine at a G/A mismatch (Xu 1996¹⁸²). The enzyme thymidine DNA glycosylase, isolated from the human cell line HeLa, cleaves the thymidine at G/T mismatches (Hsu 1994¹⁸³). In practice, a probe is used comprising the wild-type sequence of interest. The probe is hybridized to DNA, or cDNA corresponding to mRNA of interest. Once duplex formation has reached completion, a DNA mismatch repair enzyme is added to the reaction, and the products of the cleavage are detected by, for instance, separating reactants by denaturing polyacrylamide gel electrophoresis.

(e) Alterations in Electrophoretic Mobility

In another exemplary embodiment, variations in electrophoretic mobility are used to identify genetic lesions, by standard techniques, such as single strand conformation polymorphism (SSCP) (Miterski 2000¹⁸⁴, Jaeckel 1998¹⁸⁵, Cotton 1993, Ibid, Hayashi 1992¹⁸⁶). Dilute preparations of radiolabeled single-stranded DNA fragments of test and control nucleic acids, separately, are denatured by heat and permitted to renature slowly. Upon renaturation, single stranded nucleic acids in the dilute solutions form secondary structures. Each molecule forms internal base paired regions depending on each molecule sequence. Consequently, wild-type and mutant sequences, otherwise identical except for regions of mutation, form different secondary structures. Each preparation is separated in adjacent lanes by electrophoresis through native polyacrylamide gels while preserving the secondary structure formed during renaturation. Alterations in electrophoretic mobility reveal differences between wild-type and mutant oligonucleotides as small as single nucleotide differences. Following electrophoresis the radiolabeled nucleic acids are detected by autoradiography or by phosphorimager analysis. A variation of this assay employs RNA rather than DNA.

In a related exemplary embodiment, wild-type and mutant DNA molecules are separated by electrophoresis through polyacrylamide gels containing a gradient of denaturant. The method, termed “denaturing gradient gel electrophoresis,” (DGGE) (Myers 1985B, Ibid) is commonly used to detect differences between similar oligonucleotides. Prior to analysis, test DNA is often modified by addition of up to 40 base pairs of GC rich DNA through PCR. The relatively stable region, termed “GC clamp,” ensures only partial denaturation. A variation of the assay employs a temperature rather than chemical gradient of denaturant.

(f) Selective Oligonucleotide Hybridization

In another embodiment, selective hybridization involves the use of synthetic oligonucleotide primers prepared to carry a known mutation in a central position. Primers are then mixed with test DNA under conditions permitting hybridization for perfectly matched molecules (Lipshutz 1995¹⁸⁷, Guo 1994¹⁸⁸, Saiki 1989¹⁸⁹). The allele specific oligonucleotide (ASO) hybridization method can be used to test a single mutation per reaction mixture, or many different mutations if the ASO is first immobilized on a suitable membrane. The technique, termed “dot blotting,” permits rapid screening of many mutations when nonimmobilized DNA is first radiolabeled to permit visualization of the immobilized hybrids.

(g) Allele Specific Amplification

Under certain conditions, polymerase extension occurs only if there is a perfect match between primer and the 3′ terminus of the 5′, left-most or upstream region of a sequence of interest. Therefore, in another embodiment, allele specific amplification, a selective PCR amplification based assay, a synthetic oligonucleotide primer is prepared carrying a mutation at the center, or extreme 3′ end of the primer, such that mismatch between primer and test DNA prevents, or reduces efficiency of the polymerase extension during amplification (Efremov 1991¹⁹⁰, Gibbs 1989¹⁹¹). A mutation in the test DNA is detected by a change in amplification product concentration relative to controls, or, in special cases, by the presence or absence of amplification products.

A variation of the assay introduces a novel restriction endonuclease recognition site in the expected mutation region to permit detection by restriction endonuclease cleavage of the amplification products (see also above).

(h) Protein Truncation Test

Another embodiment uses the protein truncation test (PTT). If a mutation introduces a premature translation stop site, PTT offers an effective detection assay Geisler 2001¹⁹², Moore 2000¹⁹³, van der Luijt 1994¹⁹⁴, Roest 1993¹⁹⁵). In this assay, RNA is isolated from sample cells or tissue and converted to cDNA by reverse transcriptase. The sequence of interest is amplified by the PCR, and the products are subjected to another round of amplification with a primer carrying a promoter for RNA polymerase, a sequence for translation initiation. The products of the second round of PCR are subjected to transcription and translation in vitro. Electrophoresis of the expressed polypeptides through sodium dodecyl sulfate (SDS) containing polyacrylamide gels reveals the presence of truncated species arising from the presence of premature translation stop sites. In a variation of this assay, if the sequence of interest is contained within a single exon, DNA rather than cDNA can be used as PCR amplification template.

(i) General Comments

Any tissue or cell type expressing a sequence of interest may be used in the described assays. For instance, bodily fluids, such as blood obtained by venipuncture or saliva, or non-fluid samples, such as hair, or skin, may be used. Samples of fetal polynucleotides collected from maternal blood, amniocytes derived from amniocentesis, or chorionic villi obtained for prenatal testing, can also be used.

Pre-packaged diagnostic kits containing one or more nucleic acid probes, primer set, and antibody reagent may be useful in performing the assays. Such kits are designed to provide an easy to use instrument especially suitable for use in the clinic.

The assays may also be applied in situ directly on the tissue to be tested, fixed or frozen. Typically, such tissue is obtained in biopsies, or surgical procedures. In situ analysis precludes the need for nucleic acid purification.

While the exemplary assays described so far primarily permit the analysis of one nucleic acid sequence of interest, they may be also used to generate a profile of multiple sequences of interest. The profile may be generated, for example, by employing Northern blot analysis, a differential display procedure, or reverse transcriptase-PCR (RT-PCR).

In addition to nucleic acid assays, antibodies directed against a mutated polynucleotide, or polypeptide product of a mutated polynucleotide may be used in various assays (see below).

(10) Assaying Methylation Status of DNA

(a) Sodium Bisulfite Method

In one exemplary embodiment, the methylation status of DNA sequences can be determined by first isolating cellular DNA, and then converting unmethylated cytosines into uracil by treatment with sodium bisulfite, leaving methylated cytosines unchanged. Following treatment, the bisulfite is removed, and the chemically treated DNA is used as a template for PCR. Two parallel PCR reactions are performed for each DNA sample, one using primers specific for the DNA prior to bisulfite treatment, and one using primers for the chemically modified DNA. The amplification products are resolved on native polyacrylamide gels and visualized by staining with ethidium bromide followed by UV illumination. Amplification products detected from the sodium bisulfite treated samples indicate methylation of the original sample.

Specifically, this assay can be used to asses the methylation status of DNA binding sites of a polypeptide of interest, such as GABP, p300, CBP, etc.

(11) Assaying Protein Phosphorylation

(a) Western Blot with Antiphosphotyrosine

In one exemplary embodiment, protein phosphorylation is measured using anti-phosphotyrosine antibodies (for instance, antibodies available from Santa Cruz Biotechnology, catalog numbers sc-508 or sc-7020). Cultured cells are lysed by boiling in detergent-containing buffer. Proteins contained in the cell lysate are separated by electrophoresis through SDS polyacrylamide gels followed by transfer to a nylon membrane by electrophoresis, a process termed electroblotting (Burnett 1981¹⁹⁶). Prior to incubation with antibody, the membrane is incubated with blocking buffer containing the nonionic detergent Tween 20 and nonfat dry milk as a source of protein to later block adventitious binding of specific antibodies to the nylon membrane. The immobilized proteins are then reacted with anti-phosphotyrosine antibodies and visualized after reaction with a secondary antibody conjugated to horse radish peroxidase. Exposure to hydrogen peroxide in presence of the chromogenic indicator diaminobenzidine produces visible bands where secondary antibodies are bound, thereby enabling their localization.

A variation of this assay can be performed with antibodies directed against phosphothreonine (for instance, those available from Santa Cruz Biotechnology, catalog number sc-5267) or a host of phosphorylated molecules. Sources of available phosphoprotein specific antibodies include, but are not limited to, Santa Cruz Biotechnology of Santa Cruz, Calif., Calbiochem of San Diego, Calif. and Chemicon International, Inc. of Temecula, Calif.

The protein phosphorylation detection assays may be employed before and/or after treatment with an agent of interest to detect changes in phosphorylation status of a polypeptide, or group of polypeptides. Moreover, detection of changes in phosphorylation status of polypeptides of interest may be used to monitor efficacy of a therapeutic treatment or progression of a chronic disease.

(b) Immunoprecipitation

In one complementary embodiment, the relative levels of phosphorylated and nonphosphorylated forms of any particular protein may be measured. The levels of the phosphorylated forms are measured as described above. Nonphosphorylated proteins are measured by first immunoprecipitating all forms of the protein of interest with a specific antibody directed toward that protein. The immune complexes are then analyzed by Western blotting as described. Comparison of the levels of total protein of interest to those of the phosphorylated forms provides some insight into the relative levels of each form of the polypeptide of interest.

(12) Assaying Gene Activation and Suppression

(a) Co-Transfection with Report Gene to Identify Transactivators

In one exemplary embodiment, interactions between regulatory proteins and a DNA sequence of interest can be revealed through co-transfection of two recombinant vectors. The first vector carries a full length cDNA for the regulatory factor driven by a promoter known to be active in the transfected cells. The second recombinant vector carries a reporter gene driven by the DNA sequence of interest. Examples of suitable reporter genes include chloramphenicol acetyltransferase (CAT), luciferase or β-galactosidase (Virts 2001¹⁹⁷). Detection of reporter gene expression by methods known in the art (see examples below) indicates transactivation of the DNA sequence of interest by the regulatory factor.

Transfection of appropriate recombinant vectors can be mediated either with calcium phosphate (Chen 1988¹⁹⁸) or DEAE-dextran (Lopata 1984¹⁹⁹). In one exemplary embodiment, exponentially growing cells are exposed to precipitated DNA. A DNA solution, prepared in 0.25M CaCl₂ is added to an equal volume of HEPES buffered saline and incubated briefly at room temperature. The mixture is then placed over cells and incubated overnight to permit DNA adsorption and absorption into the cells. The next day the cells are washed and cultured in complete growth medium.

In a related exemplary embodiment, calcium chloride precipitation is replaced with DEAE-dextran as a carrier for the DNA to be transfected. Growth medium is made 2.5% with respect to fetal bovine serum (FBS) and 10 μM with respect to chloroquine. The medium is prewarmed, and DNA is added prior to addition of DEAE-dextran. The mixture is then added to exponentially growing cells, and incubated for 4 hours to allow DNA adsorption. The transfection medium is replaced by a 10% solution of DMSO causing the DNA to enter the cells. The cells are incubated for 2-10 hours. The DMSO solution is then replaced by growth medium, and the cells are incubated until assayed for exogenous gene expression.

CAT

Detection of CAT gene expression is achieved by mixing lysates of the cells in which the reporter gene has been co-transfected along with a recombinant vector carrying the putative activating factor with ¹⁴C-labeled chloramphenicol (Gorman 1982²⁰⁰). Acetylated and unacetylated forms of the compound, the latter resulting from enzymatic degradation of the substrate by expressed CAT, are separated by thin layer chromatography and visualized by autoradiography. Measurements of each radiolabeled species are attained by densitometric analysis of the autoradiograph, or by direct phosphorimager analysis of the chromatograph.

Luciferase

Detection of expressed luciferase is achieved by exposure of transfected cell lysates to the luciferase substrate luciferin in presence of ATP, magnesium and molecular oxygen (Luo 2001²⁰¹). The presence of luciferase results in transient release of light detected by luminometer.

β-Galactosidase

Detection of β-galactosidase gene expression is achieved by mixing cell lysates with a chromogenic substrate for the enzyme, such as o-nitrophenyl-β-D-galactopyranoside (ONPG), or a chemiluminescent substrate containing 1,2 dioxetane. Products of the catalytic degradation of the chromogenic substrate are easily visualized, or alternatively, quantified by spectrophotometry, while the products of the chemiluminescent substrate are detected by luminometer. The latter assay is especially sensitive and can detect minute levels, or minute changes in levels of β-galactosidase reporter gene expression.

These assays were applied to demonstrate binding of GABP to the promoter regions of a number of genes including the retinoblastoma gene (Sowa 1997²⁰²), CD18 (Rosmarin 1998, ibid), cytochrome C oxidase Vb (Sucharov 1995²⁰³) and the prolactin gene (Ouyang 1996²⁰⁴).

(b) Co-Transfection with Reporter Gene to Identify Trans-Acting Repressors

These assays can be applied to assess trans-acting factors which potentially repress rather than stimulate reporter gene expression. In this embodiment, putative repression factors are expressed from a recombinant vector in cells which carry a reporter gene driven by a constitutively active promoter which may interact with the repression factor. The assays described above are applied to determine whether expression of the repression factor reduces reporter gene activity.

(13) Assaying Gene Expression Levels

(a) Northern Blot Analyses

In one exemplary embodiment, the relative expression levels of a gene of interest are measured by Northern blot analysis (Ausubel 1999, Ibid). RNA is isolated from untreated cells and cells after treatment with an agent expected to modulate gene expression. The RNA is separated by electrophoresis through a denaturing agarose gel, typically incorporating the denaturant formaldehyde, and transferred to a nylon membrane. Immobilized RNA is hybridized to a radiolabeled DNA probe representing the gene of interest. Bound radiolabel is visualized by autoradiography. Levels of bound radiolabel can be quantified by scanning the resulting autoradiograph with a densitometer and integrating the area under the traces. Alternatively, incorporated radiolabel can be quantified by phosphorimager analysis of the blot itself.

(b) RT-PCR

In a related embodiment, RNA is isolated from similarly treated cells. The RNA is then subjected to reverse transcription (RT) and amplification by the polymerase chain reaction (PCR) in the presence of radiolabeled deoxynucleotides. The amplification products are resolved by gel electrophoresis and visualized by autoradiography. Levels of incorporated radiolabel can be quantified by scanning the resulting autoradiograph with a densitometer and integrating the area under the traces. Alternatively, incorporated radiolabel can be quantified by phosphorimager analysis of the electrophoretic gel.

(14) Assaying Viral Replication

(a) Viral Titer

In one exemplary embodiment, viral replication is measured by titration of infectious particles on cultured host cells. Virus replication is permitted in host cells, with or without chemical treatment, or with or without co-expression of a regulatory gene, for a measured period of time. The cells are lysed by exposure to a hypotonic solution, and the lysates are subjected to a series of dilutions in isotonic buffer. Several concentrations of cell lysate are separately plated onto cultured host cells. The culture cells are incubated until the cytopathic effects (CPE) are evident. The cultured cells are then fixed and stained with a contrast enhancing dye, such as crystal violet, to facilitate identification of viral plaques. Several culture plates are counted, and the number of plaques multiplied by the appropriate dilution factor, representing the dilution from the original cell lysate. The result reveals the viral titer of the original cell lysate.

(b) In situ PCR

In a related exemplary embodiment, a latent, low copy number virus can be detected with the polymerase chain reaction in situ (Staskus 1994²⁰⁵). Cells grown either in suspension culture or on a solid substrate are fixed and permeabilized. PCR reaction components, including synthetic primers complementary to the gene of interest, Taq polymerase, deoxyribonucleotides, are then added to the cells and subjected to thermal cycling typical of PCR. The amplification products, retained in each cell, are detected by in situ hybridization with appropriately labeled DNA probes. An exemplary detection method involves hybridization with radiolabeled probes followed by autoradiography. Similarly, hybridization probes may be nonradioactively labeled by including digoxygenin-1-dUTP into the PCR reaction. Incorporated label is detected either enzymatically or chemically.

(15) Assaying Cell Morphology and Function

(a) Light Microscopy

In one exemplary embodiment, the morphology of cells is ascertained by microscopic examination. Statin trypan blue can distinguish between living and dead cells (Schuurhuis 2001²⁰⁶). Living cells, with intact cellular membranes, exclude trypan blue while dead cells, with leaky, or perforated outer membranes, permit trypan blue to enter the cytoplasm. Following treatment, examination by phase contrast microscopy reveals the proportion of dead vs. living cells. Similarly, cellular morphology can be ascertained by examination with phase contrast microscopy, with or without prior staining, with, for example, crystal violet, to enhance contrast. Such examination reveals morphologies common to known cell types, and concomitantly reveals irregularities present in the cell population under examination.

b) Functional Assessment by Immunocytochemistry

In a related exemplary embodiment, the functional status of a given cell population may be determined by treatment with specific antibodies. Cells are dehydrated and fixed with a series of methanol washes using increasing concentrations of methanol. Once fixed, the cells are exposed to cell-type specific antibodies. Examples of suitable antibodies include, but are not limited to, anti-filaggrin for epidermal cells, anti-CD4 for T cells, thymocytes and monocytes, and anti-macrosialin for macrophages. After incubation with differentiation-specific marker antibodies, fluorescently labeled secondary antibodies specific for the first antibody are added. Bound secondary antibodies are visualized by illumination with light of appropriate wavelength to excite the bound fluorochrome followed by microscopic examination. The use of different antibodies, each conjugated to a different fluorochrome, permits the identification of multiple differentiation-specific antigens simultaneously in the same population of cells.

(16) Assaying Cellular Oxidation Stress

(a) Cellular Indicators

In one exemplary embodiment, oxidation stress within a population of cells can be measured by assaying the activity levels of certain indicators such as lipid hydroperoxides (Weyers 2001²⁰⁷). Cell lysates are prepared and mixed with the substrate 1-napthyldiphenylphosphiine (NDPP). Any resulting oxidized form of the substrate, ONDPP, can be quantified by high performance liquid chromatography (HPLC). ONDPP concentration provides an indirect measure of the oxidation capacity of the cell lysate.

(b) H2DCFDA as Indicator

In another exemplary embodiment, the production of cellular reactive oxygen species can be detected by mixing cell lysates with 2′,7′-dichlorodihydrofleuoescein diacetate (H2DCFDA) (Brubacher 2001²⁰⁸). In the presence of cellular esterases, H2DCFDA is deacetlyated to produce 2′,7′-dichlorodihydrofleuoescein (H2DCF), an oxidant-sensitive indicator. Increased cellular oxidation excites the fluorogenic indicator. Using H2DCF directly can attain increased sensitivity, but caution must be exercised by one skilled in the art to ensure that none of the experimental buffers contain contaminants, such as metals, which may lead to spontaneous fluorescence.

(d) Optimization Protocols

Once a single constructive or disruptive agent (polynucleotide, polypeptide, small molecule, etc.) is identified in the manner described above, variant agents can be formulated that improve upon the original agent.

The expression “variant agents . . . that improve upon the original agent” is understood to include, but not be limited to, agents that increase therapeutic efficacy, increase prophylactic potential, increase, or decrease stability in vivo or in storage, or increase the number, or variety of post-translational modifications in vivo, including, but not limited to, phosphorylation, acetylation and glycosylation, relative to the original agent.

Variant agents are not limited to those produced in the laboratory. They may include naturally occurring variants. For example, variants with increased stability, due to alterations in ubiquitination or modifications of other target sites conferring resistance to proteolytic degradation.

(e) Treatment Protocols

(1) Introduction

According to the present invention, a polypeptide has a constructive effect if it attenuates microcompetition with a foreign polynucleotide or attenuates at least one effect of microcompetition with a foreign polynucleotide, or one effect of another foreign polynucleotide-type disruption. For example, a constructive polypeptide can reduce copy number of the foreign polynucleotide, stimulate expression of a GABP regulated gene, increase bioactivity of a GABP regulated protein, through, for instance, GABP phosphorylation and/or increase bioavailability of a GABP regulated protein, through, for instance, a reduction in copy number of microcompeting foreign polynucleotides which bind GABP. A constructive polypeptide can also, for example, inhibit expression of a microcompetition-suppressed gene, such as, tissue factor, androgen receptor, and/or inhibit replication of a p300/cbp virus (see more examples below).

Agents of the present invention are designed to address and ameliorate symptoms of chronic diseases, specifically, diseases resulting from microcompetition between a foreign polynucleotide and cellular genes. For instance, introduction of an oligonucleotide agent into a cell may disrupt this microcompetition and restore normal regulation and expression of a microcompeted gene. Agents directed against a foreign polynucleotide may reduce binding or cellular transcription factors to the foreign polynucleotide by, for instance, reducing the copy number of the foreign polynucleotide, or its affinity to the transcription factor, resulting in increased microavailability of the factors towards normal levels. Alternatively, binding of the transcription factors to cellular genes can be stimulated. In yet another exemplary embodiment, insufficient, or excessive expression of a cellular gene in a subject can be modified by administration of nucleic acids or polypeptides to the subject that return the concentration of a cellular polypeptide of interest towards normal levels.

The following section describes standard protocols for determining effective dose, and for agent formulation for use. Additional standard protocols and background information are available in books, such as In vitro Toxicity Testing Protocols (Methods in Molecular Medicine, 43), edited by Sheila O'Hare and C K Atterwill, Humana Press, 1995; Current Protocols in Pharmacology, edited by: S J Enna, Michael Williams, John W Ferkany, Terry Kenakin, Roger D Porsolt, James P Sullivan; Current Protocols in Toxicology, edited by: Mahin Maines (Editor-in-Chief), Lucio G Costa, Donald J Reed, Shigeru Sassa, I Glenn Sipes; Remington: The Science and Practice of Pharmacy, edited by Alfonso R Gennaro, 20^{th} edition, Lippincott, Williams & Wilkins Publishers, 2000; Pharmaceutical Dosage Forms and Drug Delivery Systems, by Howard C Ansel, Loyd V Allen, Nicholas G Popovich, 7^{th} edition, Lippincott Williams & Wilkins Publishers, 1999; Pharmaceutical Calculations, by Mitchell J Stoklosa, Howard C Ansel, 10^{th} edition, Lippincott, Williams & Wilkins Publishers, 1996; Applied Biopharmaceutics and Pharmacokinetics, by Leon Shargel, Andrew B C Yu, 4^{th} edition, McGraw-Hill Professional Publishing, 1999; Oral Drug Absorption: Prediction and Assessment (Drugs and the Pharmaceutical Sciences, Vol 106), edited by Jennifer B Dressman, Hans Lennernas, Marcel Dekker, 2000; Goodman & Gilman's The Pharmacological Basis of Therapeutics, edited by Joel G Hardman, Lee E Limbird, 10^{th} oh edition, McGraw-Hill Professional Publishing, 2001. See also above referenced.

(2) Effective Dose

Compounds can be administered to a subject, at a therapeutically effective dose, to treat, ameliorate, or prevent a chronic disease. Careful monitoring of patient status, using either systemic means, standard clinical laboratory assays or assays specifically designed to monitor the bioactivity of a foreign polynucleotide, is necessary to establish the therapeutic dose and monitor its effectiveness.

Prior to patient administration, techniques standard in the art are used with any agent described herein to determine the LD₅₀ and ED₅₀ (lethal dose which kills one half the treated population, and effective dose in one half the population, respectively) either in cultured cells or laboratory animals. The ratio LD₅₀/ED₅₀ represents the therapeutic index which indicates the ratio between toxic and therapeutic effects. Compounds with a relatively large index are preferred. These values are also used to determine the initial therapeutic dose. While unwanted side effects are sometimes unavoidable, they may be minimized by delivery of the therapeutic agent directly to target cells or tissues, thereby avoiding systemic exposure.

Those skilled in the art recognize that animal or cell culture models are imperfect predictors of the efficacy of any treatment in humans. Factors affecting efficacy include route of administration, achievable serum concentration and formulation of the therapeutic agent (i.e. in pill or injectable forms, administered orally or intramuscularly, with accompanying carrier, formulation of an agent adducted with a specific antibody and injected directly into the target tissue, etc.). Regardless of the method of delivery or formulation of the therapeutic agent, it is important to monitor plasma levels using a suitable technique, such as atomic absorption spectroscopy, enzyme linked immunosorbant assay (ELISA), or high performance liquid chromatography (HPLC) among others.

(3) Formulation for use

Those skilled in the art recognize a host of standard formulations for the agents described in this invention. Any suitable formulation may be prepared for delivery of the agent by injection, inhalation, transdermal diffusion or insufflation. In every case, the formulation must be appropriate for the means and route of administration.

Oligonucleotide agents, e.g. antisense oligonucleotides or recombinant expression vectors, may be formulated for localized or systemic administration. Systemic administration may be achieved by injection in a physiologically isotonic buffer including Ringer's or Hank's solution, among others. Alternatively, the agent may be given orally by delivery in a tablet, capsule or liquid syrup. Those skilled in the art recognize pharmaceutical binding agents and carriers, which protect the agent from degradation in the digestive system and facilitate uptake. Similarly, coatings for the tablet or capsule may be used to ease ingestion thereby encouraging patient compliance.

If delivered in liquid suspension, additives may be included which keep the agent suspended, such as sorbitol syrup and the emulsifying agent lecithin, among others, lipophilic additives may be included, such as oily esters, or preservatives may be used to increase shelf life of the agent. Patient compliance may be further enhanced by the addition of flavors, coloring agents or sweeteners. In a related embodiment the agent may be provided in lyophilized form for reconstitution by the patient or his or her caregiver.

The agents described herein may also be delivered via buccal absorption in lozenge form or by inhalation via nasal aerosol. In the latter mode of administration any of several propellants, including, but not limited to, trichlorofluoromethane and carbon dioxide, or delivery methods, including but not limited to a nebulser, can be employed. Similarly, compounds may be included in the formulation, which facilitate transepithelial uptake of the agent. These include, among others, bile salts and detergents. Alternatively, the agents of this invention may be formulated for delivery by rectal suppository or retention enema. Those skilled in the art recognize suitable methods for delivery of controlled doses.

In related embodiments, the agents may be formulated for depot administration, such as by implantation, via regulated pumps, either implanted or worn extracorporally or by intramuscular injection. In these instances the agent may be formulated with hydrophobic materials, such as an emulsification in pharmaceutically permissible oil, bound to ion exchange resins or as a sparingly soluble salt.

In every case, therapeutic agents destined for administration outside of a clinical setting may be packaged in any suitable way that assures patient compliance with regard to dose and frequency of administration.

Administration of the agents included in this invention in a clinical situation may be achieved by a number of means including injection. This method of systemic administration may achieve cell-type specific targeting by using a nucleic acid agent, described herein, modified by addition of a polypeptide which binds to receptors on the target cell. Additional specificity may be derived from the use of recombinant expression vectors which carry cell- or tissue-type specific promoters or other regulatory elements. In contrast to systemic injection more specific delivery may be achieved by means of a catheter, by stereotactic injection, by electorporation or by transdermal electrophoresis. Many suitable delivery techniques are well known in the art.

In an alternative embodiment the therapeutic agent may be administered by infection with a recombinant virus carrying the agent. Similarly cells may be engineered ex vivo which express the agent. Those cells may themselves become the pharmaceutical agent for implantation into the site of interest in the patient.

f) Diagnosis Protocols

Diagnosis may be achieved by a number of methods, well known in the art, using as reagents sequences of a foreign polynucleotide, disrupted gene or polypeptide, or a gene or polypeptide in a disruptive or disrupted pathway, or antibodies directed against such polynucleotides or polypeptides. Those reagents may be used to detect and quantify the copy number, level of expression or persistence of expression products of a foreign polynucleotide, disrupted gene or gene susceptible to microcompetition with a foreign polynucleotide.

Diagnostic methods may employ any suitable technique well known in the art. These include, but are not limited to, commercially available diagnostic kits which are specific for one or more foreign polynucleotides, a specific disrupted gene, a disrupted polypeptide, a gene or polypeptide in a disruptive or disrupted pathway, or an antibody against such polynucleotides or polypeptides. Well known advantages of commercial kits include convenience and reproducibility due to manufacturing standardization, quality control and validation procedures.

(1) Detection and Quantification of Polynucleotides

In one exemplary embodiment, nucleic acids, DNA or RNA, are isolated from a cell or tissue of interest using procedures well known in the art. Once isolated, the presence of a foreign polynucleotide may be ascertained by any of a number of procedures including, but not limited to, Southern blot hybridization, dot blotting and the PCR, among others. Mutations in those polynucleotides may be detected by single strand conformation analysis, allele specific oligonucleotide hybridization and related and complementary techniques. Alternatively nucleic acid hybridization with appropriately labeled probes may be performed in situ on isolated cells or tissues removed from the patient. Suitable techniques are described, for example, Sambrook 2001 (ibid), incorporated herein in its entirety by reference. Control cells and tissues are compared in parallel to validate any positive findings in clinical samples.

If the nucleic acid molecules specific to foreign polynucleotides or disrupted genes, or genes in disrupted or disruptive pathways are in low concentration, preferred diagnostic methods employ some means of amplification. Examples of suitable procedures include the PCR, ligase chain reaction, or any of a number of other suitable methods well known in the art.

In one exemplary embodiment of a diagnostic technique employing nucleic acid hybridization, RNA from the cell of interest is isolated and converted to cDNA (using the enzyme reverse transcriptase of avian or murine origin). Once cDNA is prepared, it is amplified by the PCR, or a similar method, using a sequence specific oligonucleotide primer of 20-30 nucleotides in length. Incorporation of radiolabeled nucleotides during amplification facilitates detection following electrophoresis through native polyacrylamide gels by autoradiography or phosphorimager analysis. If sufficient amplification products are attained, they may be visualized by staining of the electrophoretic gel by ethidium bromide or a similar compound well known in the art.

(2) Detection and Quantification of Polypeptides

Antibodies directed against foreign polypeptides, disrupted polypeptides, or polypeptides in disrupted or disruptive pathways, may also be used for the diagnosis of chronic disease. Diagnostic protocols may be employed to detect variations in the expression levels of polypeptides or RNA transcripts. Similarly, they may be used to detect structural variation including nucleic acid mutations and changes in the sequence of encoded polypeptides. The latter may be detected by changes in electrophoretic mobility, indicative of altered charge, or by changes in immunoreactivity, indicative of alterations in antigenic determinants.

For diagnositic purposes, protein may be isolated from the cells or tissues of interest using any of many techniques well known in the art. Exemplary protocols are described in Molecular Cloning: A Laboratory Manual, 3rd Ed (Third Edition), by Joe Sambrook, Peter MacCallum and David Russell (Cold Spring Harbor Laboratory Press 2001), incorporated herein by reference in its entirety.

In a preferred embodiment, detection of a foreign polypeptide molecule, or a cellular disrupted polypeptide molecule, or a polypeptide in a disruptive or disrupted pathway is achieved with immunological methods, including immunoaffinity chromatography, radial immunoassays, radioimmunoassay, enzyme linked immunsorbant assay, etc. These techniques, quantitative and qualitative, all well known in the art, exploit the interaction between specific antibodies and antigenic determinants on the target molecule. In each assay, polyclonal or monoclonal antibodies, or fragments thereof, may be used as appropriate.

Immunological assays may be employed to analyze histological preparations. In a preferred embodiment, tissue or cells of interest are treated with a fluorescently labeled specific antibody or an unlabeled antibody followed by reaction with a secondary fluorescently labeled antibody. Following incubation for sufficient time and under appropriate conditions for antibody-antigen interaction, the label may be visualized microscopically, in the case of either tissues or cells, or by flow cytometry, in the case of individual cells. These techniques are particularly suitable for antigens expressed on the cell surface. If they are not on the cell surface, the cells or tissue to be analyzed must be treated to become permeable to the diagnostic antibodies. In addition to the detection of antigens on the material studied, the distribution of that antibody will become evident upon microscopic examination. All immunological assays involve the incubation of a biological sample, cells or tissue, with an appropriately specific antibody or antibodies. These and other suitable diagnostic methods are familiar to those skilled in the art.

In an alternative embodiment, immunological techniques may be employed which involve either immobilized antibodies or immobilizing the cells to be analyzed on, for example, synthetic beads or the surface of a plastic dish, typically a microtiter plate (see above).

Immobilization of antibodies or cells to be analyzed is achieved through the use of any of several substrates well known in the art including, but not limited to, glass, dextran, nylon, cellulose, and polypropylene, among others. The actual shape or configuration of the substrate may vary to suite the desired assay. For example, polystyrene may be formed into tissue culture or microtitre plates, dextran may be formed into beads suitable for column chromatography, or polyacrylamide may be coated onto the inner surface of a glass test tube or bottle. These and related carriers and configurations are well known and can be tested for utility by those skilled in the art.

Detection of bound antibodies is achieved by labeling, either directly or indirectly, through the use of a secondary antibody specific for the first. The label may be either a chromophore, which responds to excitation by a specific wavelength of light, thereby producing fluorescence, or it may be an enzyme, which reacts with a chromogenic substrate to produce detectable reaction products. Common florescent labels include fluorescineisothiocyanate (FITC), rhodamine and trans-1-bromo-2,5-bis-(3-hydroxycarbonyl-4-hydroxy)styrylbenzene (BSB), among others. Enzymes commonly conjugated with antibodies include, but are not limited to, alkaline phosphatase, horse radish peroxidase and β-galactosidase. Other alternatives are available and well known in the art.

In a related embodiment, the antibody is labeled with a fluorescent metal, for example ¹⁵²Eu, which can be attached directly to the primary or secondary antibody in an immunoassay. Alternatively, the antibody may be labeled with a chemiluminescent compound, such as luminol, isoluminol or imidazole or a bioluminiscent compount, such as luciferin or aequorin. Subsequent reaction with the appropriate substrate for the labeling compound produces light, which is detectable visually or by fluorimetry.

(3) Imaging of Diseased Tissues

Under suitable circumstances, foreign polypeptides, polypeptides expressed from disrupted genes, or from genes in a disruptive or disrupted pathway, may be detected on the surface of affected cells or tissues. In these instances the level and pattern of expression may be visualized and used to both diagnose disease and to guide and gauge therapy. For example, in atherosclerosis, such disrupted polypeptides may include, but are not limited to CD18 or tissue factor (see more details in examples below).

Under these circumstances, antibodies, monoclonal or polyclonal, which specifically interact with proteins expressed on the cell surface, may be used for the diagnosis of chronic disease and for monitoring treatment efficacy. In this embodiment, an appropriate antibody or antibody fragment is labeled with a radioactive, fluorescent, or other suitable tag prior to reaction with the biomaterial to be assayed. Conditions for reaction and visualization are well known in the art and permit analyses to be carried out in vitro as well as in situ. In a preferred embodiment, antibody fragments are used for in situ or in vitro assays because their smaller size leads to more rapid accumulation in the tissue of interest and more rapid clearing from that tissue following the assay. A number of suitable and appropriate labels may be used for the assays in this invention that are well known in the art.

(g) Clinical Trials

Another aspect of current invention involves monitoring the effect of a compound on a treated subject in a clinical trial. In such a trial, the copy number of a foreign polynucleotide, its affinity to cellular transcription factors, the expression or bioactivity of a disrupted gene or polypeptide, or expression or bioactivity of a gene or polypeptide in a disrupted or disruptive pathway, may be used as an indicator of the compound effect on a disease state.

For example, to study the effect of a test compound in a clinical trial, blood may be collected from a subject before, and at different times following treatment with such a compound. The copy number of a foreign polynucleotide may be assayed in monocytes as described above, or the levels of expression of a disrupted gene, such as tissue factor, may be assayed by, for instance, Northern blot analysis, or RT-PCR, as described in this application, or by measuring the concentration of the protein by one of the methods described above. In this way, the copy number, or expression profile of a gene of interest or its mRNA, may serve a surrogate or direct biomarker of treatment efficacy. Accordingly, the response may be determined prior to, and at various times following compound administration. The effects of any therapeutic agent of this invention may be similarly studied if, prior to the study, a suitable surrogate or direct biomarker of efficacy, which is readily assayable, was identified.

### B. EXAMPLES

The current view holds that, in vivo, viral proteins are the sole mediators of viral effects on the host cell. Such proteins include, for example, the papilomavirus type 16 E6 and E7 oncoproteins, SV40 large T antigen, Epstein-Barr virus BRLF1 protein, and adenovirus E1A. The possibility that presence of viral DNA in the host cell can directly impact cell function, independent of viral protein, is typically ignored. The viral “protein-dependent” view is so ingrained in current research that in many cases, when a “protein-independent” effect on cellular gene expression, or other cell functions, presents itself in the laboratory, the effect is ignored. As a result, the significance of such effect, and specifically, its relation to disease is overlooked. Note that the effect of viral DNA on the cellular genome in cases of viral DNA integration which may result in mutations, deletions or methylation of host cell DNA, cannot be considered “protein-independent” since it is mediated by viral proteins, such as, HIV-1 IN protein, or retrovirus integrase. The following examples illustrate the invention. More examples can be found in patent application PCT/US01/05314, incorporated herein in its entirety by reference.

The present invention starts from the discovery that microcompetition is involved in a variety of human diseases. It is only by looking through the lens of the present invention that a discernable pattern of disease progression and symptomology is understood. From this understanding the inventor was able to develop new assays, screening regiments and treatments.

Once microcompetition was discovered to play a role in human disease, the present inventor looked back at previous work to see if it was possible to find published observations consistent with microcompetition. Having made the original discovery, the inventor has been able to piece together and relate a mosaic of individual studies and information that heretofore seemed entirely unrelated.

The present invention started as a new theory of human disease and testing the hypothesis was also performed in a novel way. Once the theory was developed, a novel mechanism of action and relationship between biochemical agents was proposed, followed by a set of predictions of the effect of modification of one or more of those biochemical agents. However, it was unnecessary to perform thousands of experiments to test the hypothesis, because others had studied the biochemical agents and recorded the effects of modifying those agents. By looking at the results of thousands of studies on dozens of biochemical agents, the set of predicions was tested and supported. Close to 800 papers are referenced in this disclosure, each providing a piece of information that forms the totality of this invention.

Much of this disclosure is similar to a mosaic. In the same way, ceramic plates or colored glass are shattered and rearranged by the mosaic artist to form a new piece of art, the applicant has similarly used pieces of information evidence gleened from work of other researchers to understand the mechanism of human disease in an entirely new way.

The present invention teaches the relationship between microcompetition and human disease. The examples section starts with detailed explanation of microcompetition. It then progresses through the affected pathways and teaches the pieced together evidence supporting the microcompetition model. Based upon this model, a series of new assays, screening regiments and treatments are described (see above). The full citation for each reference is provided at the end of the detailed disclosure and is cited in an abbreviated fashion within the text to make the disclosure more readable.

1. Discovery 1: Microcompetition

(1) Definition

The situation where DNA sequences compete for the same transcription complex will be called microcompetition. If we assume first that the cellular availability of at least one of the proteins constructing the transcription complex is limited, second that the complex binds DNA of two genes and third that binding stimulates the transcription of one of these genes, then microcompetition for the transcription complex reduces binding of the complex to the gene resulting in reduced transcription (see also above).

(2) Molecular Effect

The following studies demonstrate the effect of microcompetition on the expression of various cellular genes.

(a) Human Metallothionein-II_{A} (hMT-II_{A})

CV-1 cells were cotransfected with a constant amount of plasmid containing the hMT-II_{A} promoter (−286 nt relative to the start of transcription to +75 nt) fused to the bacterial gene coding chloramphenicol acetyltransferase (hMT-II_{A}-CAT) and increasing amounts of plasmid containing the viral SV40 early promoter and enhancer fused to the bacterial gene coding for aminoglycoside resistance (pSV2Neo). FIG. 1 illustrates the results of microcompetition between the two plasmids in terms of the relative CAT activity (relative CAT activity ═CAT activity in the presence of pSV2Neo/CAT activity in the absence of pSV2Neo).

A 2.4-fold molar excess of the plasmid containing the viral enhancer reduced 90% of CAT activity. No microcompetition was observed with the viral plasmid after deletion of the SV40 enhancer.

The efficient inhibition of hMT-II_{A} promoter activity by the SV40 enhancer suggests that the enhancer has a high affinity for a limiting transcription complex that also binds the hMT-II_{A} promoter. Moreover, although both the hMT-II_{A} promoter and the SV40 enhancer bind the Sp1 transcription factor, further studies ruled out the idea that the two plasmids compete for Sp1 or factors, which bind the TATA box (Scholer 1986²⁰⁹).

(b) Platelet Derived Growth Factor-B (PDGF-B)

JEG-3 choriocarcinoma cells were transiently cotransfected with a constant amount of PDGF-B promoter/enhancer-driven CAT reporter gene (PDGF-B-CAT) and increasing amounts of a plasmid containing either the human cytomegalovirus promoter/enhancer fused to the β-galactosidase (βgal) reporter gene (CMV-βgal) or the viral SV40 early promoter and enhancer elements fused to βgal (SV40-βgal).

FIG. 2 presents the results of microcompetition between these plasmids in terms of relative CAT activity.

Both CMV-βgal and SV40-βgal repressed the activity of PDGF-B-CAT in a concentration-dependent manner. Mutational studies of the SV40 promoter/enhancer element showed that the sequence in SV40-βgal, which competes with PDGF-B is located within the SV40 enhancer region (Adam 1996²¹⁰). However, neither a specific DNA box nor responsible transcription factors were identified.

(c) Collagen Type I α2 Chain (COL1A2)

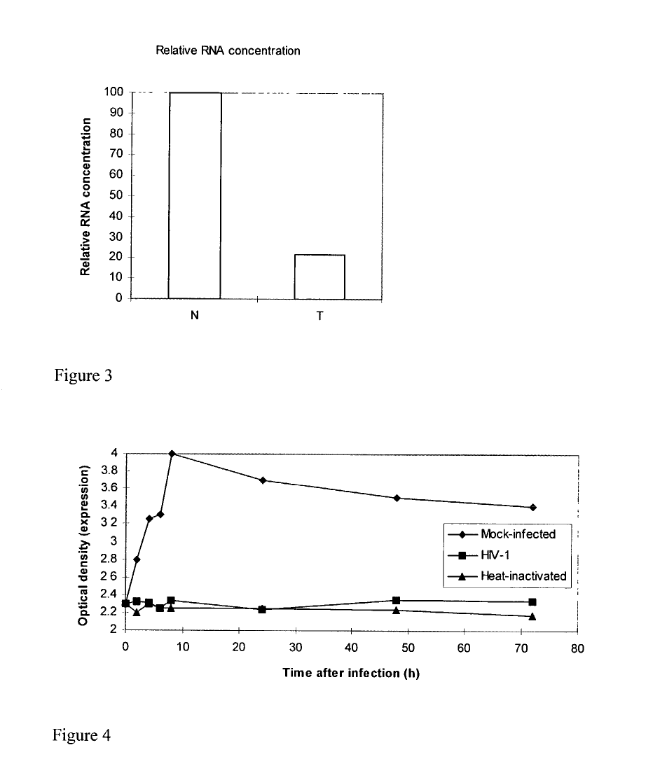

Skin fibroblasts were infected with temperature sensitive Rous Sarcoma Virus (ts-RSV). The amount of COL1A2 RNA was measured in cells grown at temperatures permissive (T) or nonpermissive (N) for transformation. FIG. 3 presents the effect of microcompetition between the virus and the cellular gene on the concentration of RNA encoded by that gene.

In skin fibroblasts the amount of COL1A2 RNA was decreased 5-fold. A similar experiment showed a reduction of 3.3-fold in the amount of COL1A1 RNA (Allebach 1985²¹¹).

A clone of SV40 transformed WI-38 human lung fibroblasts. The mRNA of the α2(I) chain was absent in the SV40 transformed WI-38 fibroblasts, whereas the mRNA of the α1(I) chain was detected on the same blot. The study eliminated a few possible reasons for the reduced expression of the α2(I) chain in the infected cells. The chromosomes, which normally carry the α2(I) and α1(I) genes, appeared to be perfectly normal. Restriction mapping of the α2(I) gene in the transformed cells did not show any gross insertion of the viral genome within the gene or its promoter. Methylation analysis of the promoter and 3′ regions of the gene did not reveal any detectable hypermethylation (Parker 1989²¹²).

Normal cells synthesize the standard form of collagen type I consisting of two α1(I) chains and one α2(I) chain. Tumors caused by the polyomavirus, on the other hand, mainly synthesize a α1(I) trimer (Moro 1977²¹³). A high concentration of trimer was also found in SV40 transformed WI-38 human lung fibroblasts (Parker 1992²¹⁴). Microcompetition mainly decreases the expression of the α2(I) chain (see Allebach 1985 and Parker 1989 above). Consequently, the relative shortage of the α2(I) chain in infected cells stimulates formation of the α1(I) trimers.

(d) CD18 (β₂ Leukocyte Integrin)

Human monocytes were infected with human immunodeficiency virus type 1 (HIV-1). The surface expression of CD18, CD11a, CD11b, CD11c, CD58, CD62L, CD54, and CD44 was measured in HIV-1 infected cells and mock-infected cells. The extent and kinetics of CD11a, CD11b, CD11c CD58 and CD62L expression were similar in HIV-1 infected cells and mock-infected cells. CD18, CD54, and CD44 showed a significant decrease in expression in the HIV-1 infected cells. When monocytes were treated with a heat-inactivated HIV-1 virus, the expression of CD54 and CD44 was similar to the expression in mock-infected cells, however, the expression of CD18 was reduced. Consider the results in FIG. 4.

According to Le Naour, et al., (1997²¹⁵) “treatment with heat-inactivated virus shown that regulation of CD18 expression is dependent on early HIV-related regulatory mechanisms whereas regulation of CD44 and CD54 requires viral events taking place after retrotranscription of viral RNA.”

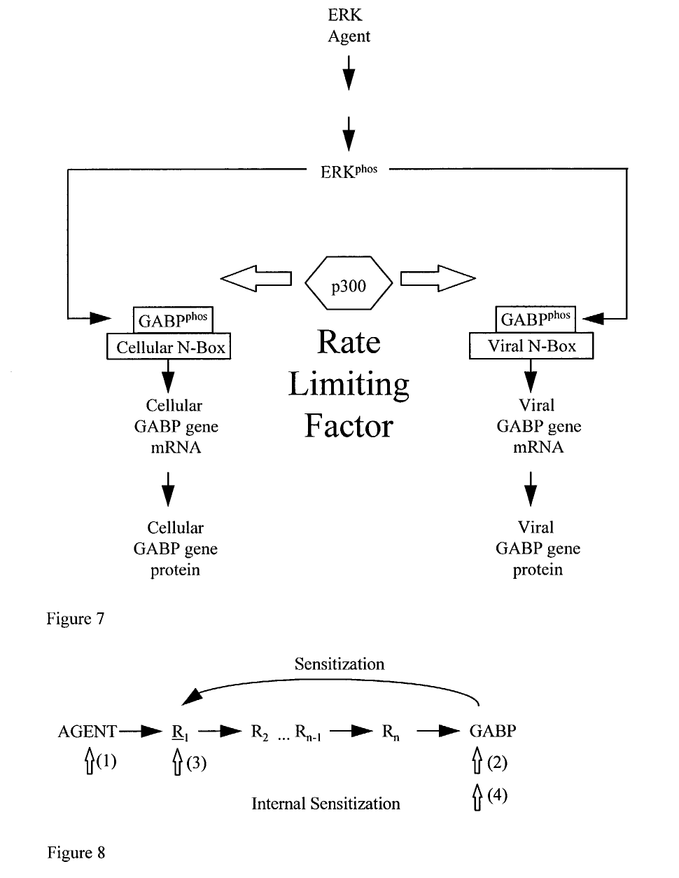

Adult T-cell leukemia (ATL) is etiologically associated with the human T-cell leukemia virus type 1 (HTLV-1). The mRNA of CD18 was measured in three human T-cell acute-lymphoblastic-leukemia cell lines, MOLT-4, Jurkat and CEM negative for HTLV-1, four T-cell lines, MT-2, TCL-Kan, C91/PL and C8166, which were established by transformation with HTLV-1, one T-cell line, TOM-1, derived from an HTLV-1 carrier and positive for HTLV-1, and four cell lines, MT-1, TL-Om1, H582 and HuT102, which are ATL derived T-cell lines positive for HTLV-1. Overall, non ATL derived, HTLV-1 negative cell lines showed high levels of CD18 mRNA. The non ATL derived, HTLV-1 positive cell lines showed moderate levels of CD18 mRNA. The ATL derived, HTLV-1 positive cell lines showed low levels of CD18 mRNA (Ibid, FIG. 7, Tanaka 1995²¹⁶).

Southern-blotting analysis did not reveal any gross structural changes in the CD18 gene. To test CD18 promoter activity in the ATL derived, HTLV-1 positive cell lines, TL-Om1, H582 and HuT102 were transfected with a CD18 promoter-driven CAT reporter gene. The same construct was transfected into the non ATL derived, HTLV-1 negative Jurkat cells. The results showed high CAT expression in the Jurkat cells and low CAT expression in the 3 ATL derived, HTLV-1 positive cell lines. Tanaka, et al., (1995, ibid) conclude that “the down regulation of the CD18 gene in these ATL cell lines was due to lack of transcription factor(s) necessary for CD18 gene expression.” The paper does not identify the transcription factor; neither does it provide an explanation for the reduced availability of the unknown factor(s).

The Epstein-Barr virus (EBV) selectively infects human B cells causing infectious mononucleosis (IM). Lymphoblastoid cell lines (LCLs) were derived from EBV-infected B cells obtained from normal individuals, IM patients, or by in vitro EBV transformation of normal B cells. LCLs grow as large cell clusters. In contrast, Burkitt lymphoma (BL) cells grow mostly as single cells or loose clusters. The CD18 surface expression was measured in 10 LCLs and 10 BL cell lines. Approximately one-third of the cell population in each LCL was CD18-negative. In comparison, the majority of the malignant cells in each BL cell were CD-18 negative (Patarroyo 1988²¹⁷).

In all these studies, competition between viral and cellular DNA for limiting regulatory factors reduces transcription of the CD18 gene.

(3) GABP Transcription Complex

(a) GABP

See introduction to GABP, the N-box, and examples of cellular GABP regulated genes above.

(b) p300/cbp

The coactivator p300 is a 2,414-amino acid protein initially identified as a binding target of the E1A oncoprotein. cbp is a 2,441-amino acid protein initially identified as a transcriptional activator bound to phosphorylated cAMP response element (CREB) binding protein (hence, cbp). p300 and cbp share 91% sequence identity and are functionally equivalent. Both p300 and cbp are members of a family of proteins collectively referred to as p300/cbp (see more detail above).

(c) Cellular Availability of p300 is Limited

Although p300/cbp are widely expressed, their cellular availability is limited. Several studies demonstrated inhibited activation of certain transcription factors resulting from competitive binding of p300/cbp to other cellular or viral proteins. For example, competitive binding of p300, or CBP, to the glucocorticoid receptor (GR), or retinoic acid receptor (RAR), inhibited activation of a promoter dependent on the AP-1 transcription factor (Kamei 1996, ibid). Competitive binding of cbp to STAT1α inhibited activation of a promoter dependent on both the AP-1 and ets transcription factors (Horvai 1997²¹⁸). Competitive binding of p300 to STAT2 inhibited activation of a promoter dependent on the NF-κB RelA transcription factor (Hottiger 1998, ibid). Other studies also demonstrated limited availability of p300/cbp, see, for instance, Pise-Masison 2001²¹⁹, Banas 2001, ibid, Wang 2001²²⁰, Ernst 2001²²¹, Yuan 2001²²², Ghosh 2001²²³, Li 2000²²⁴, Nagarajan 2000²²⁵, Speir 2000, ibid, Chen 2000²²⁶, and Werner 2000²²⁷.

(d) GABP Binds p300

GABP binds the p300 (Bannert 1999, ibid). GABPα binds directly to the C-terminal of p300 and much more weakly to the N-terminal. GABPβ does not bind directly to p300.

(e) Cellular Availability of GABP·p300 is Limited

Since cellular availability of p300 is limited, cellular availability of the GABP·p300 transcription complex is also limited.

(f) GABP Viruses

Many viruses bind GABP (see examples above). A virus, which binds the GABP complex, is called a GABP virus (see above).

(4) Microcompetition for GABP·p300

Since GABP·p300 is limiting, microcompetition for GABP·p300 between a GABP virus and a cellular GABP regulated gene reduces cellular availability of GABP·p300 to the cellular gene. Under such conditions, if the complex stimulates the gene transcription, the gene shows reduced transcription. If the complex suppresses the gene transcription, the gene shows increased transcription.

2. Discovery 2: GABP·p300 Binding Regulation

(1) ERK Pathway

Extracellular signals are transmitted to the nucleus in many ways. Often signal transduction occurs through activation of a kinase found in the cytoplasm. Once activated, the kinase translocates to the nucleus where it phosphorylates target transcription factors thereby modifying their capacity to regulate gene expression. For MAP kinase cascades, the signal is propagated through sequential activation of multiple kinases. These kinases amplify small input signals into large changes in output. All MAP kinases are activated by dual phosphorylation on a Thr-Xaa-Tyr motif, after which they function as proline-directed Ser/Thr kinases with minimal target sequence of Ser/Thr-Pro (Hipskind, 1998²²⁸).

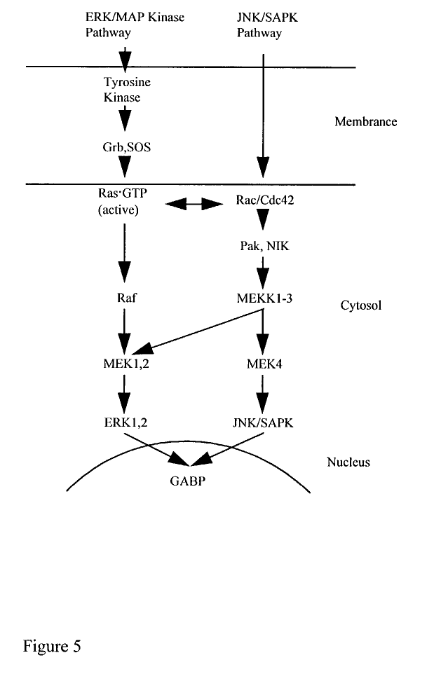

Growth factors, and other extracellular agents that support proliferation, activate the ERK (Extracellular signal-regulated kinase, previously called the MAP kinase) signaling cascade, see FIG. 5.

The kinases that make up the core of this cascade are Raf, which phosphorylates MEK, which in turn phosphorylates ERK. Raf (MAPKKK) is activated by an unclear mechanism usually dependent upon Ras. By interacting with Ras, Raf is relocalized to the membrane, which appears to be an important step for its activation. The Raf family has three known members; c-Raf (or Raf-1), B-Raf and A-Raf, and each of these proteins can function as a MAPKKK depending upon cell type. c-Raf has been generally described as the major activator. Other kinases can also function in this capacity (i.e.-MEKKs 1 and 3 and the possibility remains open for other specific activators of the ERK cascade.

Raf activates the MAPKK MEK (MEK1 and MEK2), a kinase that phosphorylates both Thr and Tyr residues in the activation motif in ERK. There are five members of the ERK family identified to date, p44ERK1, p42ERK2, ERK3, ERK4, and ERK5/BMK1 (for Big MAP Kinase). Activation results in translocation of ERK to the nucleus, where it targets transcription factors and the basal transcription complex.

Dephosphorylation at either Thy or Tyr residue inactivates ERK. There are three classes of ERK inactivators: Type 1/2 serine/threonine phosphatases, such as PP2A, tyrosine-specific phosphatases (also called protein-tyrosine phosphatase, denoted PTP), such as PTP1B, and dual specificity phosphatases, such as MKP-1. For recent reviews of the role of these classes of phosphatases in the regulation MAP kinase activity, see Camps 2000²²⁹, Saxena 2000²³⁰ and Keyse 1998²³¹. Herein the term “ERK phosphatase” denotes any phosphatase that inactivates ERK. The class of all ERK phosphatases is a super class of the above three classes of ERK inactivators.

FIG. 6 illustrates the activation of MAPK by MEK-1, a MAPKK, and deactivation of MAPK by PP2A, a serine/threonine phosphatase, PTP1B, a tyrosine-specific phosphatase, or MKP-1, a dual specificity phosphatase. A diamond represents a kinase, an ellipse, a phosphatase, an arrow, phosphorylation, and a T-headed line, dephosphorylation.

For a discussion of the JNK/SAPK pathway see below.

(2) ERK Agents

A molecule, which stimulates the phosphorylation of ERK, will be called an “ERK agent.” Some ERK agents include sodium butyrate (SB), trichostatin A (TSA), trapoxin, phorbol ester (phorbol 12-myristate 13-acetate, PMA, TPA), retinoic acid (RA, vitamin A), zinc and copper, interferon-γ (IFNγ), new differentiation factor (NDF or heregulin), estron, etradiol (E2), interleukin 1β (IL-1β), interleukin 6 (IL-6), tumor necrosis factor α (TNFα), transforming growth factor β (TGFβ) and oxytocin (OT). Consider the following evidence.

(a) Sodium Butyrate (SB), Trichostatin A (TSA) and Trapoxin

The ERK agents sodium butyrate (SB), trichostatin A (TSA) and trapoxin were tested for their effects on the major promoter (M) of human choline acetyltransferase (ChAT). The human choline acetyltransferase gene was activated by sodium butyrate, trichostatin A, and trapoxin A in transient and stable transfection studies (Espinos 1999, ibid). These agents also stimulated ERK1 and ERK2 phosphorylation. If the MAP kinase cascade is blocked with the MAP kinase kinase (MEK) inhibitor PD98059 or by overexpression of dominant-negative mutants of Ras and ERK2, activation of ChAT promoter by sodium butyrate is suppressed (Espinos 1999, ibid).

Transcriptional activation of cellular and transfected genes by histone deacetylase (HDAC) inhibitors is blocked by H7, an inhibitor of serine/threonine protein kinases. In transient transfections with the human ChAT gene, cells were treated for 1 hour with H7, and then sodium butyrate or trapoxin were added in the continued presence of H7. Under these conditions, H7 inhibited the activation by both trapoxin and sodium butyrate (Espinos 1999, ibid). Similar experiments were performed using the RSV LTR and the SV40 enhancer. Activation of these enhancer regions by sodium butyrate or trapoxin was suppressed by H7. In addition, the MEK inhibitor PD98059 blocked activation of the RSV LTR by sodium butyrate, while activation of the SV40 promoter was similarly depressed about three-fold (Espinos 1999, ibid).

Transcription of the nicotinic acetylcholine receptor (AChR) in adult muscle is restricted to the nuclei located at the neuromuscular junction. The N-box, a promoter element, contributes to this specialized synaptic expression of the AChR δ- and ε-subunits. GABP binds to the N-box in vitro. GABP subunits contain phosphorylation sites which serves as targets for MAP kinases and these kinases also mediate the heregulin-elicited stimulation of transcription of AChR genes in cultured chick myotubes. Phosphorylation studies in chick primary myotubes showed that heregulin stimulated GABPα and GABPβ phosphorylation. Both subunits of GABP are phosphorylated in vivo by MAP kinases and heregulin enhances their phosphorylation (Schaeffer 1998²³²).

(b) Phorbol Ester (Phorbol 12-Myristate 13-Acetate, PMA, TPA), Thapsigargin

The murine macrophage cell line RAW 264.7 was stimulated with thapsigargin, an endomembrane Ca(2+)-ATPase inhibitor, and TPA, the protein kinase C activator. Both thapsigargin (30 nM) and TPA (30 nM) induced phosphorylation of p44/p42 MAP kinase and production of histamine in a time- and concentration-dependent manner. The specific MEK1 inhibitor PD98059 strongly suppressed both the thapsigargin and TPA induced histamine production. Another MEK1 inhibitor, U-0126, also inhibited both the thapsigargin and TPA-induced histamine production in a concentration-dependent manner (Shiraishi 2000, ibid).

TPA induces in vitro differentiation of the pluripotent K562 human leukemia cell line. Treatment of K562 cells with TPA resulted in growth arrest, polyploidy, morphological changes, and increased cell-cell and cell-substrate adhesion. These PMA-induced changes were preceded by a rapid rise in the MEK1 activity that resulted in the sustained ERK2 activation. The MEK1 inhibitor, PD098059, reversed both the growth arrest and the morphological changes induced by TPA treatment. These results demonstrate that the TPA-induced signaling cascade initiated by protein kinase C activation requires activity of MEK/ERK signaling complex in regulating cell cycle arrest (Herrera 1998, ibid).

TPA was used to inhibit apoptosis in HL-60 cells stimulated with the JNK/SAPK activator anisomycin. An increase in ERK activity was associated with the anti-apoptotic effect. The MEK1 inhibitor, PD98059, inhibited TPA-mediated ERK activity and abrogated the anti-apoptotic effects of TPA. Moreover, inhibition of apoptosis was attenuated by pretreatment with PKC inhibitors (Stadheim 1998, ibid).

(c) Retinoic Acid (RA, Vitamin A)

Yen, et al., (1999, ibid) stated “Among the three major mitogen-activated protein kinase (MAPK) cascades—the extracellular signal regulated kinase (ERK) pathway, the c-JUN N-terminal/stress-activated protein kinase (JNK/SAPK) pathway, and the reactivating kinase (p38) pathway—retinoic acid selectively utilizes ERK but not JNK/SAPK or p38 when inducing myeloid differentiation of HL-60 human myeloblastic leukemia cells. Retinoic acid is known to activate ERK2. The present data show that this activation is selective for the MAPK pathway. JNK/SAPK or p38 are not activated by retinoic acid.”

(d) Interferon-γ (IFNγ)

IFNγ activates both ERK and PKC in human peripheral blood monocytes (Liu 1994, ibid). IFNγ also induced ERK activation in rat C6 glioma cells. In C6 glioma cells, transient expression of the dominant-negative form of c-Ha-Ras (Asn-17) abrogated IFNγ-induced ERK1 and ERK2 activation. Furthermore, the MEK1 specific inhibitor, PD98059, blocked this activation. These results indicate that p21ras and MEK1 are required for IFNγ-induced ERK1 and ERK2 activation (Nishiya 1997, ibid).

(e) Heregulin (HRG, or New Differentiation Factor, NDF)

Heregulinβ1 (HRGβ1) induced ERK activation and cell differentiation in AU565 breast carcinoma cells. ERK activation remained elevated for 2 h following high doses of HRG. The MEK specific inhibitor, PD98059, inhibited activation of ERK and completely blocked HRG-induced differentiation reversing cell growth arrest. A transient transfection of a mutant constitutively active MEK1 construct into AU565 cells induced differentiation in the absence of HRG. Treatment with HRG potentiated this response. This study indicates that HRG induces the sustained activation of the MEK/ERK pathway and that this activation is essential for inducing differentiation of AU565 cells (Lessor 1998, ibid).

HRG activated the MAP kinase isoforms p44ERK1 and p42ERK2 and the p70/p85 S6 kinase in AU565, T47D and HC11 cells. HRG stimulation caused growth arrest of the AU565 cells and proliferation of the T47D or HC11 cells. HRG also stimulated tyrosine phosphorylation and in vitro kinase activity of ErbB-2. When TPA, another ERK agent, activated PKC HRG was no longer able to activate ErbB-2 in T47D cells, blocking cell proliferation. Activation of ErbB-2 by point mutation or monoclonal antibodies also stimulated MAPK and p70/p85 S6 kinase pathways. The same monoclonal antibodies also induced AU565 cell differentiation (Marte 1995, ibid).

HRGβ2 stimulation of MDA MB-453 cells resulted in tyrosine phosphorylation of p185c-erbB2 and p180erbB4 receptors in a time- and dose-dependent fashion. Activation of ERK (>30-fold over untreated controls) was observed upon receptor(s) activation, as was the induction of the immediate early gene c-fos (>200-fold) (Sepp-Lorenzino 1996, ibid). In another study, HRGβ2, the ligand for erbB3 and erbB4 caused ERK activation and mitogensis of growth arrested T-47D human breast cancer cells. The MEK1 specific inhibitor, PD98059, completely blocked HRG-induced entry into S-phase (Fiddes 1998, ibid).

(f) Zinc (Zn) and Copper (Cu)

Egr1, an immediate early transcription factor, is induced after brain insults by an unknown mechanism. Short exposure to zinc led to sustained ERK activation (Park 1999, ibid). The MEK1 inhibitor, PD098059, inhibited ERK1/2 activation, Egr1 induction, and neuronal death by zinc. That study concluded that zinc activates ERK1/2 (Park 1999, ibid). In another study, zinc enhanced ERK activity in serum-starved Swiss 3T3 cells treated with insulin and phosphocholine (Kiss 1997, ibid).

The human bronchial epithelial cell line BEAS was exposed to noncytotoxic levels of metals including Cu and Zn. Kinase activity assays and Western blots (with phospho-specific MEK1 antibody) showed that MEK1 is activated by Cu or Zn treatment. Additional Western blots using phospho-specific ERK1/2 antibody showed that PD98059, the selective MEK1 inhibitor, blocked the metal induced phosphorylation of ERK1/2 (Wu 1999, ibid). Activity assays of another study showed a dramatic activation of ERK, JNK and p38 in BEAS cells exposed to Zn, while Cu exposure led to a relatively small activation of ERK (Samet 1998, ibid).

(g) Estron, Estradiol

Treatment of human mammary cancer MCF-7 cells with estradiol stimulates rapid and transient activation of ERK1/2. Estradiol activates the tyrosine kinase/p21ras/ERK pathway in MCF-7 cells (Migliaccio 1996, ibid).

Uterine smooth muscle from rats pretreated with estradiol-17β alone or with estradiol-17β and progesterone were tested for ERK expression and activity by immunoblotting with ERK1/2 antibodies and phosphorylation assays. Estrogen and progesterone both enhanced ERK activity (Ruzycky 1996, ibid).

In another study, immunoblot analyses and phosphorylation assays showed that estradiol-17β (E2) stimulated ERK1/2 in rat cardiomyocytes. Specifically, the activation of ERK1/2 was rapid and transient, while a rapid but sustained increase of JNK phosphorylation was observed (Nuedling 1999, ibid).

(h) Interleukin 1β (IL-1β)

Treatment with IL-1β in cultured human airway smooth muscle cells increased levels of phosphorylated ERK (p42 and p44) 8.3- and 13-fold, respectively. Pretreatment of the cells with the MEK1 inhibitor PD98059 decreased ERK phosphorylation (Laporte 1999, ibid).

IL-1β treatment of HepG2 cells activated three ERK cascades, p46/54(JNK), p38, and ERK1/2. There was maximal induction of 20-, 25-, and 3-fold, respectively, in these three cascades (Kumar 1998²³³). In another study, Western blotting and kinase assays showed that IL-1β activates ERK1/2 and p38 in islets and rat insulinoma cells (Larsen 1998, ibid).

(i) Interleukin 6 (IL-6)

The cytokine IL-6 utilizes its 80-kDa ligand-binding and 130-kDa signal-transducing subunits to trigger cellular responses. Treatment of the human B cell line, AF-10, with rIL-6 activated ERK. Activation of ERK in AF-10 cells occurred at the same time as the appearance of 42- and 44-kDa tyrosine phosphoproteins (p42 and p44) (Daeipour 1993, ibid). When AF-10 cells were induced with rIL-6 in the presence of the tyrosine kinase inhibitors, genistein and geldanomycin, ERK activation decreased. These results indicate that IL-6 activates ERK1/2.

(j) Tumor Necrosis Factor α (TNFα)

TNFα stimulates IL-6 production in renal cells in culture. Human primary mesangial cells (HMCs) and human proximal tubular (HPT) cells were treated for 24 hours with TNFα in the presence and absence of the specific p38 and ERK1/2 inhibitors SB203580 and PD98059, respectively, either alone or in combination. TNFα normally activates p38 and ERK1/2. The inhibitors SB203580 and PD98059 inhibited basal and TNFα-stimulated IL-6 production in both cell types (Leonard 1999, ibid).

(k) Transforming Growth Factor β (TGFβ)

TFGβ inhibits many epithelial cell types. Both TFGβ1 and TFGβ2 trigger rapid activation of p44MAPK in two proliferating epithelial cell lines, IEC4-1 and CCL64. Results for a third TFGβ resistant cell line, IEC4-6 showed no activation of p44MAPK after TFGβ stimulation. Resting cultures of IEC4-1 cells treated with TFGβ2 led to no significant change in either DNA synthesis or p44MAPK activity. However, addition of the growth-stimulatory combination of factors (epidermal growth factor, insulin, and transferrin (EIT)) to quiescent and proliferating IEC4-1 cells stimulated DNA synthesis and led to activation of p44MAPK. The specificity for the cellular effects of growth factors may not actually occur at the level of MAPK activation, but instead at downstream events including phosphorylation of transcriptional complexes and gene activation (Hartsough 1995, ibid).

TFGβ1 also stimulates articular chondrocyte cell growth and the formation of the extracellular matrix. In vitro kinase assays showed a rapid activation of ERK induced by TFGβ1 (Yonekura 1999, ibid). The stimulation peaked at 5 min, and dropped back to basal levels within 240 min after TFGβ1 stimulation. After 240 minutes of stimulation, the c-jun N-terminal kinase activity increased only about 2.5-fold, while there was no significant change in p38MAPK activity. PD98059 decreased TFGβ1 induced Elk1 phosphorylation in a dose-dependent manner (Yonekura 1999, ibid).

(l) Oxytocin (OT)

Oxytocin (OT) treatment triggers the rapid phosphorylation of ERK2 in Chinese hamster ovary (CHO) cells (Strakova 1998, ibid). The MEK1 specific inhibitor, PD98059, significantly reduced OT-stimulated prostaglandin (PGE) synthesis (Strakova 1998, ibid). Oxytocin receptors (OTRs) are found in a number of human breast tumors and tumor cells. In a study of breast cancer cells (Hs578T cells), OT stimulated ERK2 phosphorylation and PGE2 synthesis in Hs578T cells (Copland 1999, ibid).

The rat oxytocin receptor was transfected into Chinese hamster ovary cells. Oxytocin stimulated ERK2 phosphorylation and PGE synthesis through protein kinase C activity (Hoare 1999, ibid). Deletion of 51 amino acid residues from the carboxyl terminus of the oxytocin receptor resulted in decreased affinity for oxytocin. Cells expressing the truncated receptor showed no oxytocin-stimulated ERK2 phosphorylation or PGE synthesis (Hoare 1999, ibid).

(3) Phosphorylation of GABP

ERK phosphorylates GABPα and GABPβ but phosphorylation does not change the binding of GABPto DNA (Flory 1996, ibid, Avots 1997, ibid, Hoffmeyer 1998²³⁴, Tomaras 1999²³⁵).

Phosphorylation is known to increase binding or stabilize the complex of p300 and other transcription factors, such as NF-κB unit p65 and Bbf (Zhong 1998²³⁶, Bevilacqua 1997²³⁷). The following sections present evidence consistent with the discovery that ERK phosphorylation of GABP leads to increased binding of p300 to GABP to stabilize the GABP·p300 complex.

(a) ERK Phosphorylation Increases N-Box DNase-I Hypersensitivity

Histone acetylation occurs post-translationally, and reversibly, on the ε-NH₃+ groups of lysine residues embedded in the N-terminal tails of core histones. Histone acetyltransferases (HATs) transfer the acetyl moiety from acetyl coenzyme A to the ε-NH₃+ groups of internal lysine residues. Introduction of the acetyl group to lysine neutralizes the positive charge, increases hydrophobicity and leads to unfolding of chromatin (Kuo 1998²³⁸). Histone hyperacetylation correlates with sensitivity to digestion by deoxyribonuclease I (DNase-I) (Hebbes 1994²³⁹). Moreover, binding of a transcription complex with HAT activity to DNA enhances DNase-I hypersensitivity around the DNA binding site. p300 has HAT enzymatic activity so that binding the GABP·p300 complex enhances DNase-I hypersensitivity around the N-box.

Porcine peripheral blood mononuclear cells (PBMC) were stimulated with the ERK agent TPA. The treatment consistently enhanced DNase-I hypersensitivity of the third intron enhancer of the TNFα gene (Kuhnert 1992²⁴⁰). The major transcription factor that binds the enhancer site in the third intron of TNFα gene is GABP (Tomaras 1999, ibid). TPA treatment phosphorylated ERK, which in turn phosphorylated GABP. Phosphorylation of GABP increased binding of p300. It is therefore likely that the HAT activity of p300 acetylated the histones and enhanced DNase-I hypersensitivity of the third intron enhancer.

(b) ERK Phosphorylation Synergizes with p300 Stimulation

Human neuroepithelioma CHP126 cells were transfected with a construct containing the promoter of human choline acetyltransferase (ChAT) gene fused to the luciferase reporter gene (ChAT-luciferase). The cells were stimulated with the ERK agent trapoxin which increased luciferase expression 8-fold. In a second experiment the cells were transfected with an expression vector carrying full-length p300. p300 expression increased luciferase expression 5- to 10-fold. In a third experiment the cells were transfected with p300 and stimulated with trapoxin. The combined treatment increased luciferase expression 94-fold (Espinos 1999, ibid). Trapoxin phosphorylated ERK, which in turn phosphorylated GABP. The combinded effect of GABP phosphorylation and p300 transfection on transcription was more than additive.

The greater than additive increase in transcription demonstrates that two stimulators act in the same pathway, or in pathways that merge, to increase trascription from a single promoter. If the stimulators were acting independently, the largest possible level of transcription from the two together would be the sum of the two pathways, with each stimulator increasing transcription as if the other were not present (Herschlag 1993²⁴¹). A compeling interpretation of the “more than additive” results above is that phosphorylation of GABP increased binding of p300.

(c) Inhibition of ERK Phosphorylation Blocks p300 Stimulation

H7 is an inhibitor of serine/threonine protein kinases. ERK, a serine/threonine protein kinase is therefore inhibited by H7. Activation of the ChAT promoter by either the ERK agent trapoxin or the ERK agent sodium butyrate was inhibited by 40 [M of H7. Activation of the ChAT promoter by p300 was also inhibited by H7 in a dose-dependent manner. H7 also suppressed the synergistic activation of the ChAT promoter triggered by trapoxin and p300 (Espinos 1999, ibid). Inhibition of GABP phosphorylation decreased binding of p300, which reduced trascription.

(d) Inhibition of p300 Binding Blocks Stimulation by ERK Phosphorylation

GABP binds p300 in between amino acids 1572 and 2370 (Bannert 1999, ibid) while the adenovirus E1A protein binds p300 between amino acids 1572 and 1818 (Eckner 1994²⁴²). E1A and GABP, therefore, share an overlapping binding site on p300. By displacing GABP from p300, E1A reduces the effectiveness of GABP phosphorylation. Activation of the SV40 minimal promoter and the ChAT promoter by the ERK agent sodium butyrate and by p300 was suppressed by adenovirus E1A protein (Espinos 1999, ibid).

ERK phosphorylation of GABP increases transcription. Raf-1, a kinase involved in the ERK pathway, works with GABP to stimulate HIV-1 promoter activity (Flory 1996, ibid). These results support the idea that Raf-1 activates GABPα-and GABPβ-mediated gene expression. Further tests showed that GABP is phosphorylated in vivo by Raf-1 kinase activators (e.g. serum and TPA) and constitutive versions of Raf-1 kinase. The basal phosphorylation level of GABPα and GABPβ increased 2- to 4-fold after stimulation with serum and TPA (Flory 1996, ibid). To identify kinases of GABPα and β, bacterially expressed GABPα and β proteins were tested as substrates in in vitro kinase assays. Raf-1 did not phosphorylate GABP subunits in vitro, but phosphorylation of both GABPα and GABPβ was detected in the reaction mixture containing MEK1, ERK2, GABPα, and GABPβ. ERK1 yielded similar results. A kinase-inactive ERK1 did not phosphorylate GABPα and β (Flory 1996, ibid). These results suggest that ERK1 directly phosphorylates both GABPPα and GABPPβ.

A DNA segment in the upstream region of the human IL-2 gene contains a transcription enhancer (−502 to −413). Wich binds the transcription factor GABPα and GABPβ at −462 nt to −446 nt (designated ERE-B) and −440 to −424 nt (designated ERE-A) (Avots 1997, ibid) respectively.

GABP is a target of the MAP signal transduction pathway in T cells. c-Raf enhances IL-2 induction through GABP factors. Co-transfection of a CAT reporter gene controlled by the distal enhancer with GABPα and β expression vectors into cells showed an increase in CAT activity. Mutation of one or both ERE motifs abrogated the induction, underscoring the important functional role of GABP binding for induction of the distal enhancer. These data indicate that the c-Raf mediated increase of IL-2 induction is, at least partially, mediated by the GABP factors binding to the two ERE motifs (Avots 1997, ibid). According to Avots, et al., there appears to be an important role for the MAP pathway in induction of GABP factors binding to and controlling the distal IL-2 ERE enhancer motifs in T cells (Avots 1997, ibid).

(4) ERK Agents and Microcompetition

The relationship between ERK signaling and microcompetition is summarized in FIG. 7.

Microcompetition between a GABP virus and cellular DNA reduces the availability of GABP to cellular genes. Let [N-boxy] denote the cellular concentration of viral N-boxes. Let [GABP_{c}] and [GABPᵥ] denote the concentration of GABP bound to cellular genes and viral DNA, respectively. [GABPᵥ] is a function of [N-boxy]. For every [N-boxy]>0, microcompetition reduces [GABP_{c}]. An ERK agent phosphorylates GABP and stimulates p300 binding. If [N-boxy] is fixed, the ERK agent stimulates the transcription of GABP stimulated genes and suppresses the transcription of GABP inhibited genes.

Fixed [N-boxᵥ] seems to hold in cases of latent infection. In such cases, ERK phosphorylation of GABPᵥ stimulates the formation of N-boxᵥ·GABPᵥ·p300 complexes. However, there is no increase in viral replication, which might have further reduced the availability of p300 to cellular genes and diminished or even canceled the ERK effect.

(5) JNK/SAPK Pathway

(a) Phosphorylation of GABP

Another signaling pathway, which phosphorylates GABP, is JNK/SAPK (see a figure of pathway in ERK pathway section above). Consider the following study.

To study the effects of JNK/SAPK on GABP, in vivo, HEK-293, human embryonic kidney cells were transfected with GABPα and GABPβ expression vectors alone, or in combination with SAPKβ expression vector and metabolically labeled with [³²P]orthophosphate. The cells were treated with anisomycin to strongly activate SAPK without affecting ERK activity. The results showed increased phosphorylation of both GABPα and GABPβ. The phosphorylation was further increased with SAPKβ overexpression (Hoffmeyer 1998, ibid, FIG. 5A and B). The study next tested the ability of these kinases to phosphorylate GABP in vitro, using ERK as a positive control. In vivo activated and immunopurified GST-tagged SAPKβ, but not Flag-tagged p38, phosphorylated both subunits of GABP (Ibid, FIG. 6B). Bacterially expressed, purified, and preactivated GST-SAPKαI also phosphorylated both GABP subunits in vitro like GST-c-Jun (Ibid, FIG. 6C). Both activated SEK and 3pK did not phosphorylate GABP. Next, the study tested another JNK/SAPK isozyme, JNK1/SAPKγ. In addition to ERK, untreated or TPA/ionomycin-stimulated A3.01 cells (a human T lymphoma cell line) phosphorylated both GABPα and GABPβ in vitro (Ibid, FIG. 6A). Based on these results, Hoffmeyer, et al., concluded that “the ability of three different isoforms of JNK/SAPK (SAPKα, SAPKβ, and JNK1) to phosphorylate GABP in vitro, in combination with the in vivo phosphorylation of GABP upon SAPK activation by anisomycin, suggests that GABP is targeted by JNK/SAPK-activating pathways.”

3. Discovery 3: N-box·GABP Binding Regulation

(1) Redox Regulation of GABP N-Box Binding

Oxidative stress decreases binding of GABP to the N-box, reduces transcription of GABP stimulated genes and increases transcription of GABP suppressed genes. Consider the following study.

Mouse 3T3 cells were treated for 2 h with diethyl maleate (DEM), a glutathione (GSH)-depleting agent, in the presence or absence of N-acetylcysteine (NAC), an antioxidant and a precursor of GSH synthesis. Following treatment, the cells were harvested, and nuclear extracts were prepared in the absence of a reducing agent. GABP DNA binding activity was measured by EMSA analysis using oligonucleotide probes containing a single N-box (AGGAAG) or two tandem N-boxes (AGGAAGAGGAAG). Treatment of 3T3 cells with DEM resulted in a dramatic decrease in formation of the GABP heterodimer (GABPαGABPβ), (Martin 1996²⁴³, FIG. 2A, lane 2) and heterotetramer (GABPα₂GABPβ₂), (Ibid, FIG. 2A, lane 6) complexes on the single and double N-box. Inhibition of GABP DNA binding activity by DEM treatment was prevented by simultaneous addition of NAC (Ibid, FIG. 2A, lanes 4 and 8). The reduction of GABP DNA binding activity was not due to loss of GABP protein since the amount of GABPα and GABPβ1 was unaffected by DEM or NAC treatment. Treatment of nuclear extracts prepared from DEM-treated 3T3 cells with dithiothreitol (DTT), an antioxidant restored GABP binding activity. Treatment of 3T3 nuclear extracts with 5 mM GSSG nearly abolished GABP DNA binding. Based on these observations Martin et al., concluded that GABP DNA binding activity is inhibited by oxidative stress, i.e. GSH depletion. The study also measured the effect of DEM treatment on expression of transiently transfected luciferase reporter constructs containing a TATA box with either upstream double N-box or C/EBP binding site (Ibid, FIG. 4). DEM treatment had no effect on luciferase expression from C/EBP-TA-Luc after 6 or 8 h treatment (Ibid, FIG. 4). However, DEM treatment of cells transfected with double N-box-TATA-Luc, resulted in a 28% decrease in luciferase expression after 6 h and a 62% decrease after 8 h (Ibid, FIG. 4). Based on these results, Martin et al., concluded that glutathione depletion inhibits GABP DNA binding activity resulting in reduced expression of GABP-regulated genes.

These results demonstrate that oxidative stress decreases GABP binding to the N-box which in turn decreases transcription of a GABP stimulated gene and increases transcription of a GABP repressed gene.

(2) Microcompetition as “Excess Oxidative Stress”

Microcompetition for GABP also decreases binding of GABP to the N-box. Take a GABP regulated gene sensitive to oxidative stress through GABP only¹. The effect of microcompetition on the transcription of this gene is similar to the effect of oxidative stress. In other words, for this gene, microcompetition can be viewed as “excess oxidative stress.” ¹ Oxidative stress also modifies binding of other transcription factors, such as AP1, and NF-κB.

4. Discovery 4: Molecular Effects of Microcompetition

(1) Signaling

Let a GABP kinase be any enzyme that phosphorylates GABP. Since GABP is a new concept, we sometimes revert to ERK instead of GABP kinase. However, in such cases, unless specified, ERK actually means GABP kinase.

(a) Sensitization by GABP

The statement “A stimulates B” means that A stimulates the expression of B either directly or indirectly. Let “AGENT” be a GABP kinase agent which activates the transcription factor GABP. Let GABP stimulate the expression of a protein P. Let [AGENT]₁ and [AGENT]₂ be two concentrations of AGENT with corresponding concentrations [P]₁ and [P]₂. The intensity of signal [AGENT]₁ relative to [AGENT]₂ is equal to [AGENT]₁/[AGENT]₂=[P]₁/[P]₂. The intensity of an ERK signal is measured by its effect on transcription of the protein P.

Let AGENT be a GABP kinase agent, which activates the transcription factor GABP. Let (AGENT, GABP) denote the signaling pathway that leads from AGENT to GABP. Every protein R, such that R is an element of the signalling cascade (AGENT, GABP) will be called an “ERK receptor for AGENT.” In other words, AGENT activates the R protein, which in turn activates GABP. For example, the leptin long receptor is an ERK receptor for leptin, and metallothionein is an ERK receptor for zinc.

Let AGENT be a GABP kinase agent. If there is a protein R in the signalling cascade (AGENT, GABP), such that AGENT stimulates the expression of R, the (AGENT, GABP) pathway will be called “sensitized” and R will be called the “sensitized receptor,” denoted R. Sensitization increases the intensity of a given signal by increasing the number of receptors available to be activated by a given amount of GABP kinase agent.

Let R be a sensitized receptor in (AGENT, GABP). If the expression of R is stimulated by GABP, R will be called an “internally sensitized receptor.” Consider FIG. 8.

An increase in AGENT stimulates the phosphorylation of GABP (step 1 and 2 in the figure). The phosphorylated GABP stimulates the transcription of R₁, the sensitized receptor (step 3). The new R₁ receptors increase the sensitivity of the pathway to a change in the concentration of the GABP kinase agent, that is, increase the probablity of binding between the GABP kinase agent and R₁. The increased binding further increases the number of phosphorylated GABP molecules (step 4) in a positive feedback mechanism.

In the pathway (OT, OTR, GABP), the receptor OTR is stimulated by GABP (Hoare 1999, ibid). In (zinc or copper, hMT-II_{A}, GABP), hMT-II_{A} is a receptor stimulated by GABP (see discussion above). In the pathway (LPS, CD18, GABP), CD18 is a receptor stimulated by GABP (Rosmarin 1998, ibid). In the pathway, (IL-2, IL-2Rβ, γc, GABP), IL-2Rβ and γc are two receptors stimulated by GABP (Lin 1993, ibid, Markiewicz 1996, ibid).

According to the definition of an ERK receptor, GABP is also an ERK receptor. In addition, some GABP kinase agents increase the expression of GABP turning GABP from an ERK receptor into a sensitized receptor. Consider the following examples.

GABPβ and γ are similar proteins that differ only by homodimerization section in the C-terminal region. Antibodies that are not specific to the C-terminus bind both proteins. Such antibodies are not sensitive enough to identify a relative change in their expression. However, since GABPβ and GABPγ are almost always bound to GABPα, and since GABPβ is an activator and GABPγ is a suppressor (Suzuki 1998, ibid), an increase in GABPα with an increase in gene expression indicates an increase in the GABPβ concentration relative to γ.

IFNγ

Evidence suggests that interferon-γ (IFNγ) regulates GABP DNA binding by increasing the amount of the GABP proteins present in bone marrow-derived macrophages (BMDM) nuclei. IFNγ treatment of BMDM leads to induction of the binding activity (Tomaras 1999, ibid). Since the GABPβ and GABPγ are almost always bound to GABPα, (Suzuki 1998, ibid), an increase in P most likely corresponds to an increase in GABPα.

The increase in DNA binding activity correlates with an increase in immunodetectable GABPα (Tomaras 1999, ibid). The essential sites for activity of GABP within the third intron of TNFα map to a highly conserved tandem repeat of ets-transcription factor binding sites. Mutations in the ets site within the intron inhibited this activity. A dominant-negative ets plasmid also completely negated this cooperativity. It was determined that a GGAA sequence repeat is a transcriptionally active site, which interacts with an ets transcription factor. Specifically, GABP binds to this region. GABP binding activity is increased by treatment with IFNγ in BMDM (Tomaras 1999, ibid).

Heregulin

Heregulin increases GABPα expression specifically (Schaffer 1998, ibid). Western blot analysis of heregulin treated and non-treated cells showed that heregulin treatment leads to a 2-fold increase in the protein level of GABPα, while the GABPβ protein level was unaffected (Schaffer 1998, ibid).

PMA

Bottinger, et al., 1994²⁴⁴ defined the minimal defined promoter for CD18 (p2 integrin) expression in myeloid and lymphoid cells by generating 5′ and 3′ deletion constructs of a segment ranging 785 bp upstream and 19 bp downstream of a major transcription start site. The region extending from nucleotides −302 to +19 supported cell-restricted and phorbol ester-inducible expression. Two adjacent promoter regions, from nt −81 to −68 (box A) and −55 to −41 (box B), were revealed by DNase-I footprinting of this region. DNA-binding proteins that interact with box A and box B were identified through electrophoretic mobility shift assays. Using box A as a probe yielded a major complex, designated BA-1, which increased in intensity after phorbol ester-induced differentiation of the cells. The complex was also detected using the radiolabeled box B element. The complex is homologous to GABP. Antiserum specific to GABPα or GABPβ abrogated binding of BA-1, while antisera to other ets-transcription factors had no effect (Bottinger 1994) thereby demonstrating the specificity of this interaction.

Expression of CD18 corresponds to the DNase-I protection profiles observed in vitro, suggesting that the complexes that bind to the protected elements mediate tissue specific expression of the CD18 gene. In T cells, the BA-1 complex forms over the box A and box B elements and is apparently responsible for the DNase-I protection profiles seen. Despite the formation of the same complex in the HeLa CD18 negative cell line, there is no observed DNase-I protection (Bottinger 1994, ibid).

In T cells the expression of GABPα and GABPβ increase. Since GABPαβ is an activator, Bottinger observed increased expression of CD18 and DNase-I protection on the CD18 promoter. In HeLa cells GABPα and GABPγ increase. Since GABPαγ is a suppressor, Bottinger observes no expression of CD18 and little DNase-I protection on the CD18 promoter.

(b) Resistance

(i) Hypothesis

(a) Resistance

Traditionally, there are two definitions of resistance, cellular level resistance and patient level resistance.

Cellular level resistance: Let L denote a ligand and O a cell. Let L produce the effect Y in O. The cell O will be called “L resistant” if a given concentration of L produces a smaller Y effect in O relative to control.

Patient level resistance: Let L denote a ligand. A patient will be called “L resistant” if the patient shows elevated levels of L relative to controls. Patient level resistance is sometimes called hyper-L-emia. Example: Insulin resistance as observed in late onset (type II) diabetes and hyperinsulinemia.

(b) Control

Let AGENT be a GABP kinase agent and let C be a protein. If the expression of AGENT depends on the expression of C, C will be called a “control” for AGENT. If an increase in C represses the expression of AGENT, or increases its degradation, C will be called a “negative control” and the effect on AGENT termed “feedback inhibition.”

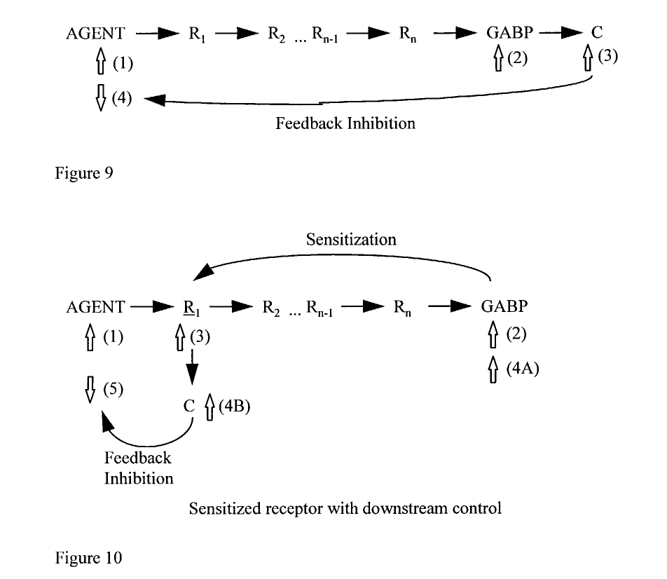

Let AGENT be a GABP kinase agent with the (AGENT, GABP) pathway. If GABP stimulates C, C will be called a “GABP stimulated” control. Consider FIG. 9.

AGENT phosphorylates GABP (step 1 and 2). GABP increases the transcription of C (step 3). C decreases the expression of the GABP kinase agent (step 4).

(c) Microcompetition Causes Resistance

Cellular Level Resistance

Let AGENT be a GABP kinase agent with the (AGENT, GABP) pathway. Let AGENT produce the effect Y in the cell O. Let the Y effect be dependent on transcription of a GABP regulated gene X in O. Under microcompetition in O, a given concentration of AGENT produces a smaller concentration of X and a smaller Y effect.

Patient Level Resistance

Let AGENT be a GABP kinase agent with the (AGENT, GABP) pathway. Let C be a negative control for AGENT which is also GABP stimulated. Microcompetition for GABP elevates the concentration of AGENT. As a GABP kinase agent, AGENT phosphorylates the pool of GABP molecules. Phosphorylation of GABP increases C, which in turn represses AGENT. However, microcompetition reduces the size of the GABP pool, or the amount of GABP available to stimulate C. Therefore, microcompetition diminishes the increase in the control C, which lessens the repression effect on A. In the above figure, the size of the arrow in step 2 would be smaller, hence the size of the arrow in step 3 would be smaller as would be that of the arrow in step 4.

Note that the control C in the above figure is down stream from GABP. What if the control is positioned between the GABP kinase agent and GABP? Would microcompetition cause patient level resistance in such a pathway?

Let R be an internally sensitized receptor in (AGENT, GABP) with C as a negative control for AGENT. If R stimulates C(C is downstream from R), microcompetition for GABP elevates the concentration of AGENT. This is illustrated by FIG. 10.

AGENT phosphorylaes GABP (step 1 and 2). GABP increases the transcription of R₁ (step 3). R₁ increases the effect on GABP (step 4A) and increases the expression of the control C (step 4B), which then decreases the expression of the GABP kinase agent (step 5). Microcompetition decreases the size of the arrows in step 2, 3, 4A, 4B and 5.

If the control is down stream from the sensitized receptor, microcompetition causes patient level resistance.

Consider the following two pathways (OT, OTR, GABP), (zinc or copper, hMT-II_{A}, GABP) as examples. In these pathways, the sensitized receptor directly binds the GABP kinase agent. Therefore, the control must be down stream from the sensitized receptor, and the pathways must show patient level resistance under microcompetition. This conclusion can be reached independent of any information about the control. The pathway (LPS, CD18, GABP) is similar. Elicitation of a bioequivalent reaction requires a higher concentration of LPS in a cell infected by a GABP virus compared to a non infected cell. The pathway (IL-2, IL-2Rβ, γc, GABP) is different (see below).

Let the set {(AGENTᵢ, GABP, Cᵢ)} include all pathways with a GABP kinase agent AGENTᵢ and control Cᵢ downstream from GABP. For all AGENTᵢ, microcompetition for GABP reduces the expression of Cᵢ, which, in steady state, increases the concentration of AGENTᵢ. Using the resistance terminology, it can be said that microcompetition for GABP causes cells infected with a GABP virus to show AGENTᵢ patient level resistance.

(2) Oxidative Stress

Microcompetition intensifies the effects of oxidative stress (see chapter on atherosclerosis).

(3) Transcription

(a) Retinoblastoma Susceptibility Gene (Rb)

(i) GABP is an Activator of Rb

Notations:

Rb represents the retinoblastoma susceptibility gene

pRb represents the retinoblastoma susceptibility protein

The Rb promoter includes a N-box at (−198,−193). Several experiments were performed in which plasmids were produced. pXRP1 included the normal (−686,−4) segment of the Rb promoter. pXRP3 included the same segment with a mutated N-box and RBF-1×4 included 4 copies of the Rb N-box as promoter. All promoters controled expression of the luciferase (luc) reporter gene. Cotransfection of hGABPα and hGABPβ1 expression plasmids with pXRP1 into SL2 Drosophila cells showed a 10-fold increase in reporter gene activity. Cotransfection with RBF-1×4 showed a 13-fold increase. Cotranfection with pXRP3, the mutated N-box, showed no increase (Sowa 1997²⁴⁵). Based on these observations, and other results, Sowa, et al., concluded that hGABP has a strong transactivating effect on the Rb gene promoter, suggesting that hGABP is the main transactivator for the core promoter element of the Rb gene.

(ii) Rb is a Microcompetition-Repressed Gene

GABP viruses microcompete with the Rb promoter for GABP. Therefore, viral infection of cells decreases Rb expression. Moreover, the higher the concentration of viral DNA, the greater the decrease in Rb expression.

(b) Breast Cancer Type 1 Gene (BRCA1)

(i) GABP is an Activator of BRCA1

The BRCA1 promoter includes three N-boxes at (−200,−178). Plasmids with point mutations in the central N-box, alone or in combination with mutations in the other N-boxes were transfected in MCF-7, a human breast cell line. The mutated plasmids showed a 3-fold reduction in promoter activity (Atlas 2000²⁴⁶, FIG. 2). Nuclear extracts from MCF-7 formed a specific complex with the N-boxes region. Through crosslinking, supershift assays and binding to recombinant GABPαβ (Atlas 2000, ibid, FIGS. 4, 5), GABPαβ was identified as the main transcription factor interacting with the N-boxes. An artificial promoter containing the multimerized N-boxes region was transactivated by cotransfection with GABPα and GABPβ1 in both MCF-7 and T47D, another human breast cell line (Atlas 2000, ibid, FIG. 6). These observations indicate that BRCA1 is a GABP stimulated gene.

(ii) BRCA1 is a Microcompetition-Repressed Gene

GABP viruses microcompete with the BRCA1 promoter for GABP. Therefore, viral infection of cells will decrease BRCA1 expression. Moreover, higher concentrations of viral DNA, lead to greater decreases in BRCA1 expression.

(c) Fas Gene (Fas, APO-1, CD95)

(i) GABP is an Activator of Fas

The Fas promoter includes two N-boxes at (−857,−852) and (−833,−828). Jurkat cells, a T cell line, were transiently transfected with a luciferase reporter gene driven by different lengths of the Fas promoter. The cells were stimulated for 10 h with anti-CD3 mAb, PMA and PMA/ionomycin. Deletion of the two N-boxes reduced activation by 50-75% (Li 1999²⁴⁷, FIG. 1). Mutation of the N-boxes also reduced stimulated luciferase activity (Ibid, FIG. 7). Cell stimulation resulted in formation of specific complexes on the N-boxes region. Mutation of the N-boxes reduced formation of these complexes (Li 1999, ibid, FIG. 4). Antibodies against GABPα and β inhibited formation of these complexes (Li 1999, ibid, FIG. 6A). Two or four copies of the Fas/GABP site (−863,−820) were inserted into a reporter plasmid carrying the pGL3/promoter. Anti-CD3 mAb, PMA and PMA/ionomycin stimulated luciferase activity 8-20 fold in Jurkat transfected cells (Li 1999, ibid, FIG. 9). Mutation of the N-boxes significantly reduced induction of luciferase activity in response to stimulation. These observations indicate that Fas is a GABP stimulated gene.

(ii) Fas is a Microcompetition-Repressed Gene

GABP viruses microcompete with the Fas promoter for GABP. Therefore, viral infection of cells decreases Fas expression. Moreover, the higher the concentration of viral DNA, the greater the decrease in Fas expression.

(d) Tissue Factor (TF) Gene

(i) Transcription

(a) ETS Related Factor(s) Repress TF Transcription

(i) ETS Related Factor(s) Bind (−363 to −343) and (−191 to −172)

A study used DNase I footprinting to map the sites of protein-DNA interaction on the (−383 to +8) fragment of the TF promoter. That study used nuclear extracts prepared from uninduced and lipopolysaccharide-induced THP-1 monocytic cells. Six regions were identified. Region number 7 (−363 to −343) and region number 2 (−191 to −172) contain an N-box. THP-1 extracts formed two complexes on a consensus N-box. Both complexes were competed with excess unlabeled N-box and 200-fold excess of a (−363 to −343) probe. The (−191 to −172) probe, although not as effective as the (−363 to −343) probe, showed approximately 30% reduction in N-box complex formation (Donovan-Peluso 1994²⁴⁸, FIG. 9).

Another study used the (−231 to −145) fragment of the TF promoter as probe. Nuclear extracts prepared from uninduced and lipopolysaccharide-induced THP-1 monocytic cells formed two complexes on the (−231 to −145) probe. To characterize the proteins that interact with the DNA sequence, the study used the sc-112× antibody from Santa Cruz Biotechnology. According to the manufacturer's literature, the antibody has broad cross-reactivity with members of the ETS family. Incubation of the antibody with the nuclear extracts abrogated the formation of the upper complex on the (−231 to −145) probe (Groupp 1996²⁴⁹, FIG. 5).

(ii) (−191 to −172) also Binds NF-κB

Monocytic THP-1 cells were stimulated with LPS for various times up to 24 h. TF mRNA increased by 30 min and reached a peak at 1 h. Levels dropped considerably by 2 h returning, eventually, to preinduction levels (Hall 1999²⁵⁰, FIG. 1). The same study conducted EMSA studies using the (−213 to −172) fragment of the TF promoter. The results showed that two complexes, indicated as III and IV, appear at 30 min, with binding reaching a peak at 1-2 h. At 4 h and later, the complexes are no longer detected. A 100-fold molar excess of a (−213 to −172) probe, or a NF-κB consensus oligonucleotide, compete with complexes III and IV (Ibid, FIG. 2B). An antibody against p65, and to a lesser extent, anti-c-Rel, supershifted complex III. These data demonstrate a transient binding of two NF-κB complexes to the (−213 to −172) fragment between 30 min and 2 h. However, the affinity of complexes for the NF-κB site was much lower than the affinity of the complexes on the adjacent proximal AP1 site.

This study also provides evidence indicating that LPS induces proteolysis of IκB and translocation of p65 and c-Rel from the cytoplasm to the nucleus. Western blot analyses showed that very little p65 was present in the nucleus in unstimulated cells. After 10 min of LPS induction, nuclear p65 begins to appear and peak at 1 h, declining again by 2 h. A concomitant decrease in cytoplasmic p65 corresponds to the observed increase in nuclear p65 (Hall 1999, ibid, FIG. 4).

(iii) The (−363 to −343) Factor(s) Repress TF Transcription

Holzmuller, et al., (1999²⁵¹) call the (−363 to −343) fragment of the TF promoter the Py-box. Deletion of the 5′-half of the Py-box increased expression of a luciferase reporter gene (Ibid, FIG. 3A and B). The relative increase was similar for LPS induced or nontreated cells and was independent of the existence of NF-κB site (Holzmuller 1999, ibid, FIG. 3C). Mutation of the N-box part of the Py-box resulted in complete loss of binding activity to the Py-box.

(b) Competition Between ETS Related Factor(s) and NF-κB for (−191 to −172)

Donovan-Peluso, et al., (1994, ibid, see above) showed that the (−191 to −172) probe was less effective in competing with the consensus N-box compared to the (−363 to −343) probe. According to the authors, the data suggest that there might be competition for binding to the (−191 to −172) fragment by NF-κB and ETS related factors. In such a case, NF-κB binding to a (−191 to −172) probe reduces the concentration of the probe available to for ETS binding. This competition can explain the reduced ability of (−191 to −172) to compete for ETS binding relative to (−363 to −343). Moreover, the NF-κB site and the N-box in the (−191 to −172) fragment overlap. The presence of overlapping sites also suggests competition where occupancy by either factor might preclude binding by the other.

(i) Microcompetition Stimulates TF Transcription

Microcompetition between a GABP virus and the TF promoter decreases the availability of the ETS related complexes in the nucleus.

NF-κB binding to (−191 to −172) increases transcription. Competition between NF-κB and ETS related factors for (−191 to −172) suggests that the decrease in availability of the ETS related factors in the nucleus increases the binding of NF-κB to the (−191 to −172) fragment and increases TF expression.

Binding of ETS related factor(s) to the (−363 to −343) fragment represses transcription. The repression is similar in extracts from untreated, or LPS- or TNF-α-induced cells. Moreover, the repression is independent of NF-κB binding. This observation suggests that the ETS related factor(s) suppress transcription in quiescent cells and maintain the rates in activated cells at a moderate level (Holzmuller 1999, ibid). The decrease in availability of the ETS related factor(s) in the nucleus reduces the (−363 to −343) repression and increases TF expression.

The GABP virus microcompetes with the TF promoter for the ETS related factor(s), therefore, viral infection of monocytes/macrophages increases TF expression. Moreover, the higher the concentration of viral DNA, the greater the increase in TF expression.

(c) GABP Viruses Increase TF Expression

(i) Transfection

A few studies measured the expression of TF relative to an internal control. Those studies used two controls, CMVβgal (Moll 1995²⁵², Nathwani 1994²⁵³) and pRSVCAT (Mackman 1990²⁵⁴). Although the studies used different transfection protocols; Moll, et al., (1995) used psoralen- and UV-inactivated biotinylated andenovirus and streptavidine-poly-L-lysine as vectors for DNA delivery, Nathwani, et al., (1994) used electoporation and Mackman, et al., (1990) used DEAT-dextran, they all report an increase in TF expression relative to a promoterless plasmid. According to Moll, et al., (1995), the cells “are being already partially activated following the transfection procedure.” The level of activation was similar in unstimulated and LPS stimulated cells. The internal controls include promoters of GABP viruses. The control promoter microcompetes with the TF promoter for ETS related factor(s). The reduced availability of ETS related factor(s) increases the transcription of the reporter gene fused to the TF promoter.

(ii) Infection

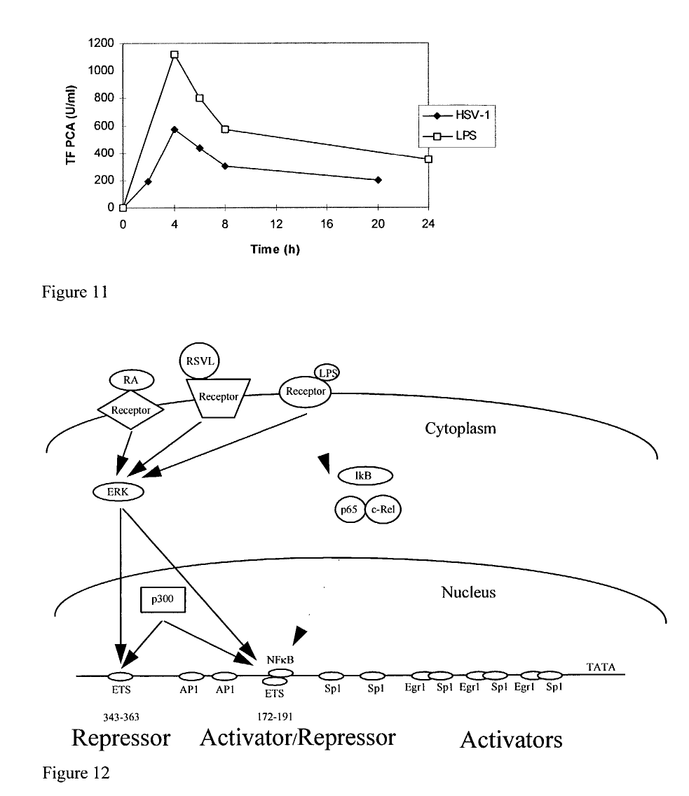

Confluent monolayers of human umbilical vein endothelial cells (HUVEC) were exposed to 0.1 μg/ml LPS for 4 hours and HSV-1. At appropriate time intervals, TF procoagulant activity (PCA) was assessed by clotting assays. FIG. 11 presents the results.

Maximal TF PCA activity was observable 4 hours after infection and was still detectable 20 hours post infection. Both the HSV infection and LPS exposure show a similar activity profile over time. However, the maximal activity induced by HSV is about a ½ of LPS. Further studies with specific blocking antibodies to human TF support the notion that the PCA is indeed due to TF.

HUVEC were also infected with HSV-1 inactivated by either ultra-violet-irradiation or heat. The cellular TF PCA was measured in lysates of control, LPS stimulated (0.1 mg/ml for 4 hours), or infected cells. Virally infected cells were maintained in culture for up to 48 hours and visually inspected for cytopathic effects as evidence for lytic infection. Obvious morphologic changes were evident in cells infected with competent virus after 18 to 24 hours. In comparison, no signs of infection were visible in cells infected with heat or UV-treated virus even after 48 hours. The TF PCA of the different treatments measured 4 hours post infection is summarized in the following table.

| | TF PCA |
|---|---|
| | (U/ml) |
| Control | 74 |
| LPS | 1753 |
| HSV-1 | 773 |
| Heated HSV-1 | 691 |
| (80° C. × 30 min) | |
| UV irradiated HSV-1 | 384 |

Virus inactivated by UV or heat is still capable of inducing TF activity (Key 1993²⁵⁵).

This study measures the effect of infection with an inactivated GABP virus on TF transcription. The reduced TF transcription is consistent with microcompetition between the viral DNA and the TF promoter for the ETS related factor(s) despite the fact that the infecting viruses were not viable.

(d) The effect of ERK Agents on TF Transcription

Many papers report the effects of c-Fos/c-Jun, c-Rel/p65, Sp1 and Egr-1 binding on TF transcription. LPS and PMA are ERK agents and, therefore, phosphorylate the ETS related factors. However, LPS and PMA also stimulate the binding of NF-κB and Egr-1, respectively, to the TF promoter. In FIG. 12, the effect of LPS on NF-κB is presented by dotted lines, and on ERK by solid lines. As such, LPS and PMA are not useful in isolating the effect of ETS phosphorylation on TF transcription. The next section presents two ERK agents, all-trans retinoic acid (ATRA) and resveratrol, which have no effect on NF-κB, Ap1 and Sp1. As ERK agents, ATRA and resveratrol phosphorylate the ETS related factor(s), stimulate the binding of p300, and, therefore, should repress TF transcription.

(i) All-Trans Retinoic Acid (ATRA)

Monocytes were incubated for 30 minutes with various doses of ATRA before LPS stimulation. ATRA inhibited LPS induction of TF expression in a dose-dependent manner (Oeth 1998²⁵⁶, FIG. 1A). The LPS induction of TF activity was also inhibited by ATRA in THP-1 monocytic cells (Ibid, FIG. 2A). Specifically ATRA reduced the basal levels of TF mRNA in unstimulated cells and abolished the LPS induction of TF mRNA (Ibid, FIG. 3A). However, ATRA did not affect DNA binding of the c-Fos/c-Jun, c-Rel/p65 or Sp1 transcription factors to the AP1, NF-κB and Sp1 sites.

(ii) Resveratrol (RSVL)

Confluent monolayers of human umbilical vein endothelial cells (HUVEC) were treated with resveratrol (100 μmol/L) for 2 hours. Following resveratrol treatment, the cells were stimulated for 6 hours with LPS, TNFα, IL-1β, or PMA. The results showed that resveratrol markedly suppressed LPS-, TNFα-, IL-1β-, and PMA-induced TF activity (Pendurthi 1999²⁵⁷, FIG. 1A). The inhibition varied from 60% to more than 90%. HUVEC monolayers were also treated with different concentrations of resveratrol (0 to 200 μmol/L) for 2 hours. Following resveratrol treatment, the cells were stimulated with TNFα, IL-1β, or PMA. The data showed that resveratrol inhibited the induction of TF expression in a dose-dependent manner. To test the effect of resveratrol in monocytes, mononuclear cell fractions were treated with various concentrations of resveratrol (0 to 100 μmol/L) for 2 hours and then stimulated with LPS (100 ng/mL) for 5 hours. The results showed that resveratrol inhibited LPS-induced TF expression in monocytes in a dose-dependent manner (Ibid, FIG. 2). To test the effect of resveratrol on TF mRNA, HUVEC monolayers were treated with various concentrations of resveratrol (0, 5, 20, 100, and 200 μmol/L) for 2 hours, and then stimulated with LPS, TNFα, IL-1β, or PMA for 2 hours. Resveratrol treatment reduced TF transcription in a dose-dependent manner. However, the reduced transcription was not due to diminished binding of c-Fos/c-Jun or c-Rel/p65 to the TF promoter. Resveratrol did not significantly change the binding of c-Fos/c-Jun to the AP-1 sites. Resveratrol treatment had no significant effect on binding activity to the AP-1 site in either unstimulated or LPS-, TNFα-, IL-1β-, or PMA-stimulated endothelial cells (Ibid, FIG. 7). Resveratrol also did not significantly change the binding of NF-κB to the TF promoter. Unstimulated cells showed little binding of NF-κB, whereas LPS, TNFα, IL-1β, or PMA induced formation of a prominent DNA-protein complex on the NF-κB site. Preincubation of cells with resveratrol (100 μmol/L), for 2 hours, had no effect on formation of the NF-κB DNA-protein complex (Ibid, FIG. 8).

Both ATRA and resveratrol are ERK agents and, therefore, phosphorylate the ETS related factor(s). In general, phosphorylation of ETS related factor(s) stimulates binding of p300. The ETS·p300 complex, when bound to the TF promoter, represses TF transcription. The repression is independent of NF-κB, Ap1 or Sp1.

(ii) Deactivation (“Encryption”) as a Function of Membrane Concentration

(a) TF Surface Dimers are Inactive

According to Bach, et al., (1997²⁵⁸), surface TF exists in two forms, monomers and dimers. Both monomers and dimers bind FVIIa. However, only monomers are active. Self-association of TF monomers prevents access to an essential macromolecular substrate-binding site. The concept of inactive (cryptic) dimers is consistent with the crystal structures of the extracellular domain of TF. The structure suggest that TF dimerization does not block FVIIa binding but covers the macromolecular substrate binding site on the opposite face of TF.

Bach, et al., (1997) provide ample evidence consistent with this model. Consider the following experiments. HL-60 cells were exposed to 10⁻⁶ mol/L PMA for various times. The intact cells were assayed for TF procoagulant activity (PCA) either before or following a brief exposure to 10 μmol/L ionomycin. In comparison to PMA treatment alone, a combined ionomycin and PMA testament resulted in a dramatic increase in expression of TF PCA (Ibid, FIG. 1). The rapid appearance of the activity suggests that de novo protein synthesis was not involved (Ibid, FIG. 2). The calcium influx activated the latent TF PCA. Also, the inhibition by calmidzaolium (CMZ) implicates calmodulin (CaM) as an essential link in the process (Ibid, FIGS. 3, 4). Moreover, FVIIa bound to TF on untreated cells as well as ionophore-treated cells (Ibid, FIG. 5, experiment 1 and 2). Thus, restricted formation of TF-FVIIa does not account for inactive (cryptic) TF PCA. The TF-FVIIa complex readily bound the pseudosubstrate tissue factor pathway inhibitor-activated factor X (TFPI-FXa) on ionophore-treated cells, but was resistant to TFPI-FXA inhibition on untreated cells. Similar inhibition on ionophore-treated cells was demonstrated with XK1, another pseudosubstrate of TF-FVIIa. These results suggest that calcium influx exposes a TFPI-FXa/XK1 binding site on TF. Lastly, HL-60 cells were treated with DTSSP, a monobifunctional amino-reactive protein shown to cross-link cell surface TF. Following the treatment, TF was immunopurified and visualized by Western blotting. The products of DTSSP cross-linking were TF dimers (Ibid, FIG. 7, lane 1, 2). When the cells were treated with ionomycin before cross-linking, almost no cross-linking was observed (Ibid, FIG. 7, lane 3). The decreased cross-linking suggests that TF does not self-associate on ionophore treated cells. Both the TF cross-linking and the encrypted TF PCA were preserved by treating the cells with CMZ before the addition of ionophore (Ibid, FIG. 7, lane 4).

(b) Increase in Surface Concentration Induces Dimers, Reduces Activity

Nemerson, et al., (1998²⁵⁹) link the surface concentration of TF with its rate of catalytic activity. To establish such a link, Nemerson and Giesen incorporated a recombinant TF (TF₁₋₂₄₃), which contained the transmembrane, but not the cytoplasmic domain, into appropriate phospholipid vesicles and measured their catalytic activity (k_{cat}). The results showed that the k_{cat}, or catalytic rate constant, which reflects the catalytic activity of each TF-FVIIa molecule, fell monotonically as a function of TF surface density. Moreover, following exposure of vesicles with high surface-density of TF (about 50 molecules of TF on the surface of a 100 nm vesicle) to a cross-linking reagent, Nemerson and Giesen were able to detect dimers and higher n-mers. Nemerson and Giesen suggested that these results are consistent with a model where clustered TF molecules have lower maximal catalytic activity compared to dispersed molecules.

To test the significance of the cytoplasmic domain in activation, Wolberg, et al., (2000²⁶⁰) transfected cells with either full length TF, or TF lacking its cytoplasmic domain. The results showed that TF activation by a calcium ionophore was independent of the cytoplasmic domain.

(c) TF Self Regulation Through Dimers

Schecter, et al., (1997²⁶¹) show the effect of agonist stimulation on TF surface concentration and activity over time. TF mRNA was barely detectable in quiescent aortic smooth muscle cells (SMC) (Ibid, FIG. 1). FCS induced a marked rise in TF mRNA levels, beginning at ˜1 h and persisting for ˜8 h. Accumulation of TF mRNA in response to PDGF BB and α-thrombin was similar to that seen with 10% FCS (Ibid, FIG. 1). To test the effect of the rise in TF mRNA on protein synthesis over time, quiescent SMC were treated with growth agonist and examined by immunostaining every hour for the first 4 h, and every 2 h for additional 20 h. Untreated quiescent SMC showed minimal TF antigen. Cells stimulated with 10% FCS, PDGF AA, or BB, or thrombin receptor peptide, produced a pronounced perinuclear staining of TF antigen beginning at 2 h and peaking at 4-6 h. At 4-6 hours, TF antigen was also detected diffusely on the ruffled edges of the plasma membrane. Perinuclear staining persisted for ˜8-10 h after stimulation, and then gradually dissipated. At 16-24 h, a patchy distribution of antigen staining near or on the membrane was noted with diminished prinuclear staining. Schecter, et al., (1997, ibid) measured the intensity of immunofluorescent staining along a line, which traverses the nucleus and connects opposite sides of the cell membrane, and displayed the results graphically. At 4 h, the graph shows a bimodal distribution with two-peaks, around the nucleus and along the membrane (Ibid, FIG. 5a, insert). At 16 h, the graph shows a much smaller peak around the nucleus and a much larger peak along the membrane (Ibid, FIG. 5b, insert).

Schecter, et al., (1997, ibid) also measured the effect of PDGF simulation on TF activity. PDGF induced an approximately fivefold increase in surface TF activity (Ibid, FIG. 7) 4-6 h after treatment, with a return to baseline by 20 h.

The temporal events reported in this study show that the initial increase in TF membrane staining (4 h post stimulation) is associated with an increase in TF activity, while the subsequent increase in membrane staining (16 h post stimulation) is associated with a decrease in TF activity. The patches of TF staining on the cell surface are most prominent at a time (10-12 h after agonist stimulation) when surface TF activity is minimal. The study finds this relationship intriguing and proposes that the patches may represent inactive TF multimers.

P-selectin (CD62P, GMP140, LECCAM-3, PADGEM) is expressed in megakaryocytes and endothelial cells. In endothelial cells P-selectin is stored in specialized granules known as Weibel-Palade (WP) bodies. After activation with inflammatory mediators, such as histamine, thrombin, or complement proteins, WP bodies fuse with the plasma membrane, resulting in increased P-selectin expression on the endothelial apical surface. One function of P-selectin is to mediate leukocyte adherence to activated endothelium.

(iii) Transcription

(a) GABP is a Repressor of β-Selectin

Two conserved N-boxes were identified in the mouse and human P-selectin genes. The mouse distal N-box is positioned at (−327,−322) and the proximal at (−104,−99). The human distal N-box is positioned at (−314,−309) and the proximal at (−103,−108). A labeled probe encoding the murine proximal N-box formed two DNA-protein complexes with nuclear extracts from BAEC (Pan 1998²⁶², FIG. 6B), bEnd.3, HEL and CHRF288 cells. Complex formation varied with different batches of nuclear extracts, characteristic of GABP binding. Competition with a HSV-1 Immediate Early (IE) N-box probe, which binds GABP, prevented complex formation with BAEC nuclear extracts (Ibid, FIG. 6D). Based on these observations, Pan, et al., concluded that the proximal N-box most likely binds the ubiquitously expressed GABP.

Mutation of the AGGAAG proximal N-box to AGCTAAG eliminated DNA-protein complex formation (Pan 1998, FIG. 6C). BAEC transfected with a reporter gene directed by the murine P-selectin promoter with the mutated N-box showed 2-10-fold increased expression compared to the wild-type promoter (Ibid, FIG. 6F). The increased transcription indicates that binding of the Ets related factor to the proximal N-box represses the P-selectin gene. Deletion of the distal N-box had no effect on reporter gene expression. The increased transcription of the mutated gene indicates that GABP is a repressor of P-selectin.

(b) Microcompetition Stimulates P-Selectin Transcription

GABP viruses microcompete with the P-selectin promoter for GABP. Therefore, viral infection of endothelial cells increases P-selectin expression. Moreover, the higher the concentration of viral DNA, the greater the increase in P-selectin expression.

(e) CD18 Gene

(i) Transcription

(a) GABP is an Activator CD18

CD18 (β₂ integrin) is a leukocyte-specific adhesion molecule. GABP binds three N-boxes in the CD18 promoter and transactivates the gene (Rosmarin 1995²⁶³, Rosmarin 1998, ibid).

(b) Microcompetition Represses CD18 Transcription

Latent infection by a GABP virus results in microcompetition between viral DNA and CD18 promoter, which decreases the expression of CD18 (Le Naour 1997, ibid, Tanaka 1995, ibid, Patarroyo 1988, ibid, see above). Moreover, the higher the concentration of viral DNA, the greater the decrease in CD18 expression.

(f) CD49d (α₄ Integrin) Gene

CD49d (α₄ integrin) is expressed in B cells, thymocytes, monocytes/macrophages, granulocytes and dendritic cells. α₄ binds PI integrin to form α₄β₁ (CD49d/CD29, VLA-4). α₄β₁ binds vascular cell adhesion molecule-1 (VCAM-1), which appears on the surface of activated endotheilal cells, and fibronectin (Fn), a major component of the extra-cellular matrix (ECM).

(i) Transcription

(a) GABP is an Activator of α₄ Integrin

Rosen, et al., (1994²⁶⁴) show that GABP binds the (−51,−46) N-box in the α₄ promoter. The binding of GABP activated transcription of the α₄ integrin gene in Jurkat cells, a T-cell line.

(b) Microcompetition Represses α₄ Transcription

Rosen, et al., (1994) show that microcompetition with an Ets binding site from the Moloney sarcoma virus long terminal repeat inhibited binding of GABP to the α₄ integrin promoter. GABP viruses microcompete with the α₄ promoter for GABP. Therefore, viral infection of macrophages decreases α₄ expression. Moreover, the higher the concentration of viral DNA, the greater the decrease in α₄ expression.

(g) Hormone Sensitive Lipase (HSL) Gene

Hormone sensitive lipase (HSL, Lipe, EC 3.1.1.3) is an intracellular neutral lipase highly expressed in adipose tissue. HSL is the rate-limiting enzyme in triacylglycerol and diacylglycerol hydrolysis. HSL also mediates cholesterol esters hydrolysis generating free cholesterol in steroidogenic tissues and macrophages.

(i) HSL is a Microcompetition-Suppressed Gene

(a) N-Box

The region −780 bp 5′ of exon B to the start of exon 1 was suggested to include potential regulatory sites of the human HSL gene in adipocytes (Talmud 1998²⁶⁵, Grober 1997²⁶⁶). This region includes 15 N-boxes. Moreover, three pairs are located within short distances of each other. The distance between the pair at (+268,+272), (+279,+285) is 5 bp or 1.0 helical turn (HT), at (+936,+942), (+964,+970) is 22 bp or 2.5 HT, and at (+1,253,+1259), (+1270,+1276) is 11 bp or 1.5 HT.

Of the dozens of known ETS factors, only GABP, as a tetrameric complex, binds two N-boxes. Typically, the N-boxes are separated by multiples of 0.5 helical turns (HT). There are 10 bp per HT. Consider the following table (based on Yu 1997²⁶⁷, FIG. 1).

| | Distance between | |
|---|---|---|
| Gene | N-boxes* | |
| Murine Laminin B2 | 26 | bp |
| | 3.0 | HT |
| Human type IV collagenase | 11 | bp |
| | 1.5 | HT |
| Human CD4 | 12 | bp |
| | 1.5 | HT |
| Murine CD4 | 12 | bp |
| | 1.5 | HT |
| Murine COX Vb | 27 | bp |
| | 3.0 | |
| Murine COX IV | 15 | bp |
| | 2.0 | HT |
| Ad2-ML | 6 | bp |
| | 1.0 | HT |

*Distance measure in bp (base pair) or HT (helical turns).

The 1.0, 2.5 and 1.5 helical turns separating the HSL N-boxes pairs is consistent with characteristic GABP heterotetramer binding.

It is interesting to note that the HSL testis-specific promoter also includes two N-boxes separated by 11 bp or 1.5 helical turns (Blaise 1999²⁶⁸). Many “TATA-less” promoters bind GABP to an N-box in their initiator element. Specifically, HSL is a TATA-less gene. Three N-boxes on the HSL gene, (+35,+41) in exon B and (+964,+970), (+1110,+1116) in intron B are conserved in the mouse HSL gene (see sequence U69543 in Talmud 1998, ibid).

(b) Transfection

The Swiss mouse embryo 3T3-L1 fibroblasts can differentiate into adipocyte-like cells. The undifferentiated cells contain a very low level of HSL activity. While differentiated adipocyte-like cells show a 19-fold increase in HSL activity (Kawamura 1981²⁶⁹).

3T3-L1 preadipocytes were induced to differentiate by incubation with insulin (10 μg/ml), dexamethasone (10 nM), and iBuMeXan (0.5 mM) for 8 consecutive days following cell confluency. HSL mRNA was measured in undifferentiated confluent controls and differentiated 3T3-Li cells transfected with the ZIPNeo vector. Although differentiated 3T3-LI cells usually show significant HSL activity, the 3T3-Li differentiated cells transfected with ZIPNeo showed decreased HSL mRNA (Gordeladze 1997²⁷⁰, FIG. 11 left). ZIPNeo carries the Moloney murine leukemia virus LTR which binds GABP. Microcompetition between the viral LTR and the HSL promoter leads to reduced expression of the HSL gene.

The following section presents the clinical effect of microcompetition.

5. Discovery 5: Clinical Effects of Microcompetition

(a) Cancer

(1) Effect of Microcompetition on Cell Proliferation and Differentiation

The current paradigm holds that, in vivo, viral proteins are the mediators of host cell manipulation. Consider, as examples, the extensive research published on the SV40 large T antigen, Epstein-Barr virus BRLF1 protein, papilomavirus type 16 E6 or E7 oncoproteins or adenovirus E1A. The possiblity of host cell manipulation independent of viral protein is ignored.² This paradigm is so ingrained that even when protein-independent manipulation presents itself in the lab, the investigators disregard its significance. Consider the following studies as examples, each uses two types of plasmids. One plasmid includes a gene of interest, cellular Rb or viral T antigen. The other plasmid includes the neomycin-resistance (Neo) gene only under the control of a viral promoter. This plasmid is regarded as “empty,” and is, therefore, used as control. All three studies report results showing a significant effect of the “empty” plasmid on cell cycle progression, increased proliferation and reduced differentiation. However, none of these studies includes any reference to these results. The results are completely ignored. ² A possible exception migh be integration of viral DNA into cellular genome. Such integration may result in mutations, deletions or methylation of in host cell DNA. However, even this manipulation of cellular function is mediated, in many cases, by viral proteins. Consider, as examples, the HIV-1 IN protein or the retrovirus integrase which mediate viral integration.

(a) Microcompetition Stimulates Proliferation

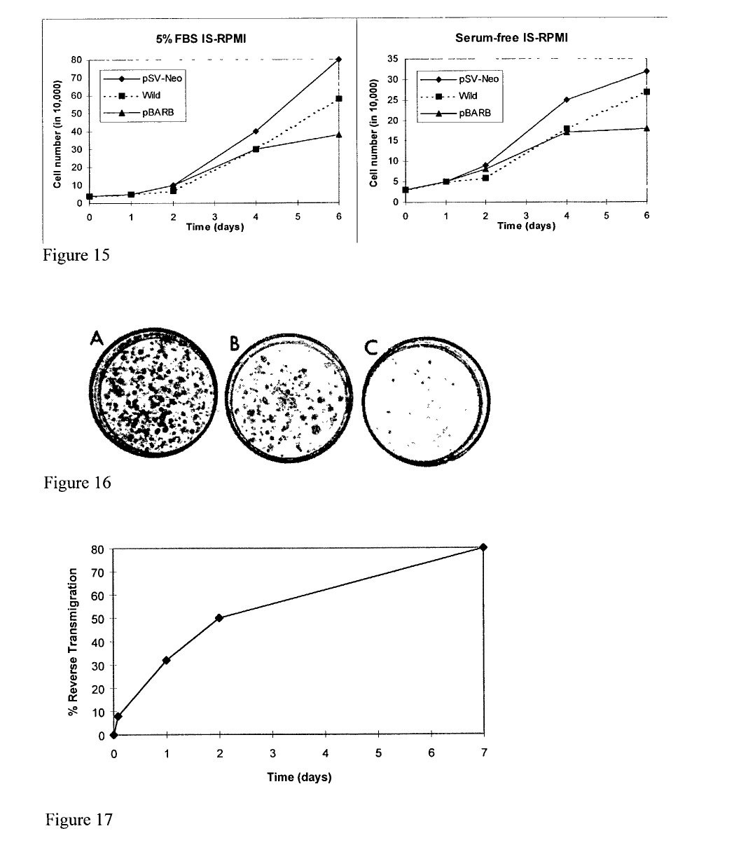

HuH-7 human hepatoma cells were transfected with pBARB, a plasmid in which the β-actin promoter regulates the expression of the Rb gene and the simian virus (SV40) promoter regulates the expression of the neomycin-resistance (neo) gene. The cells were also transfected with the pSV-neo plasmid, which only includes the SV40 promoter on the neo gene. Since pSV-neo does not include the β-actin promoter and the Rb gene, it was regarded as “empty” and was used as control. The cells were incubated in the chemically defined medium IS-RPMI with 5% FBS or serum free IS-RPMI. The number of viable cells were counted at the indicated times. The results are summarized in FIG. 15 (Awazu 1998²⁷¹, FIG. 2A). Wild means non-transfected cells. The SD is about the size of the triangular and circular symbols.

Rb transfection resulted in reduced cell proliferation at day 6 relative to non-transfected “wild” type HuH-7 cells. Transfection of the “empty” vector resulted in increased proliferation. The “empty” vector includes the SV40 promoter that binds GABP. Microcompetition between the viral promoter and cellular genes leads to increased proliferation (for the identity of the cellular genes, see below).

(b) Microcompetition Inhibits Differentiation

HSV-neo is a plasmid that expresses the neomycin-resistance gene under the control of murine Harvey sarcoma virus long terminal repeat (LTR) (Armelin 1984²⁷²). pZIPNeo expresses the neomycin-resistant gene under the control of the Moloney murine leukemia virus long terminal repeat (Cepko 1984²⁷³). PVUO carries an intact early region of the SV40 genome, which expresses the SV40 large tumor antigen and SV40 small tumor antigen (Higgins 1996²⁷⁴). The murine 3T3-L1 preadipocytes were transfected with PVUO. The cells were also transfected with HSV-neo and pZIPNeo as “empty” controls. Following transfection, the cells were cultured under differrentiation inducing conditions. Glycerophosphate dehydrogenase (GPD) activity was measured as a marker of differentiation. The results are presented in the following table (Higgins 1996, ibid, Table 1, first four lines).

| | | GPD activity |
|---|---|---|
| Vector | Cell line | (U/mg of protein) |
| None | L1 | 2,063 1,599 |
| HSV-neo | L1-HNeo | 1,519 1,133 |
| ZIPNeo | L1-ZNeo | 1,155 1,123 |
| PVU0 | L1-PVU0 | 47, 25 |

Transfection of PVUO and expression of the large and small T antigens resulted in a statistically significant decrease in GPD activity. However, transfection of the “empty” vectors, HSV-neo and ZIPNeo, although less than PVUO, also reduced GPD activity. In a t-test, assuming unequal variances, the p-value for the difference between the HSV-neo vector and no vector is 0.118, and the p-value for the difference between ZIPNeo and no vector is 0.103. Given that the sample includes only two observations, a p-value around 10% for vectors carring two different LTRs indicates a trend. Both the murine Harvey sarcoma virus LTR and the Moloney murine leukemia virus LTR bind GABP. Microcompetition between the viral LTR and the 3T3-LI preadipocyte GABP regulated genes regulating cell cycle leads to the reduced differentiation, indicated by the reduced GPD activity.

The wild-type early region of SV40 was inserted into the “empty” pZIPNeo plasmid (same plasmid as in Higgins 1996, ibid, see above). The new plasmid is called the “wild-type” (WT) and expresses the SV40 large T antigen. 3T3-F442A preadipocytes were transfected with either WT or pZIPNeo. Accumulation of triglyceride, assayed by oil red staining, was used as a marker of differentiation. Seven days postconfluence, the number of staining of cells was recorded. Consider FIG. 16. Darker staining indicates increased differentiation. The symbol (A) marks untreated F442A cells, (B), cells transfected with ZIPNeo, and (C), cells transfected with WT (Cherington 1988²⁷⁵, FIG. 4A, B and C).

Transfection with WT, the vector expressing SV40 large T antigen, reduced differentiation, see triglyceride staining in (C) and (A). However, transfection with the “empty” vector, although less than WT, also reduced differentiation, see triglyceride staining in (B) relative to (A) and (C).

pZIPNeo utilizes the Moloney murine leukemia virus long terminal (LTR), a region which binds GABP. Microcompetition between the viral LTR and the cellular genes regulating cell cycle progression leads to reduced differentiation, indicated by reduced accumulation of triglyceride.

(2) Pathogenesis

(a) Rb

(i) Hypophosphorylated Form of pRb and Cell Cycle

The cell cycle starts with a growth period (G1). Prior to a time in late G1, called R-point, the cell “decides” whether to divide or exit the cell cycle. An exit results in growth arrest, differentiation, senescence or apoptosis. A decision to divide leads to a series of orderly processes starting with DNA synthsis (S), a second growth period (G2), mitosis and cell division (M), and a return to G1. As cells progress through the cell cycle, pRb undergoes a series of phosphorylation events. In G0 and early G1, pRb is primarily unphosphorylated. As cells approach the G1/S boundary, pRb becomes phosphorylated by cyclin D/CDK4 and cyclin D/CDK6 kinases, as seen by a higher-molecular-weight species of pRb. Further phosphorylation by cyclin E/CDK2 kinase occurs in late G1. Phosphorylation is progressive and continuous throughout the S phase and into G2/M. Phosphopeptide analysis demonstrated that pRb is phosphorylated on more than a dozen distinct serine or threonine residues throughout the cell cycle (Sellers 1997²⁷⁶).

Let un-pRb denote the unphosphorylated form of pRb, hypo-pRb, the hypo or under phosphorylated form of pRb and hyper-pRb, the hyperphosphorylated form of pRb. Un/hypo-pRb denotes the set of all pRb either un or hypophosphorylated.

Accumulation of un/hypo-pRb leads to G1 arrest. This hypothesis is supported by many observations. For instance, E2F is a transcription factor associated with cell proliferation. Un/hypo-, but not hyper-pRb, binds and inactivates E2F. The cellular introduction of viral oncogenes such as HPV16 E7, adenovirus E1A, and simian virus 40 (SV40) large T antigen result in cell proliferation. These viral oncogenes bind un/hypo-, but not hyper-pRb and disable its suppressive capacity. The human osteogenic sarcoma cell line SAOS-2 lacks full length nuclear pRb protein. Transfection of the Rb gene in these cells result in G0/G1 growth arrest. Co-transfection of cyclin D2, E or A resulted in pRb phosphorylation and a release from G0/G1 arrest (Dou 1998²⁷⁷)

(ii) Rb Transcription Increases in Arrest and Differentiation

The following studies show increased Rb transcription in arrested or differentiated cells.

(a) mRNA Measurements

Murine erythroleukemia (MEL) cells are virus-transformed erythroid precursor cells which can be induced to differentiate by a variety of chemicals. MEL cells were induced to differentiate with dimethyl sulfoxide (DMSO) or hexamethylene bisacetamide (HMBA). Expression of globin was used as a marker of differentiation. The cells showed a 11- and 7-fold increase in Rb mRNA following DMSO and HMBA treatment, respectively, with maximum expression on day three of induction (Coppola 1990²⁷⁸, FIG. 1). This increase preceded the accumulation of globin mRNA, the marker of differentiation. The peak in Rb mRNA occurred simultaneously with growth arrest and terminal differentiation. Another cell line, S2 myoblasts derived from C3H10T1/2 mouse embryonic by 5-azacytidine treatment, was induced to differentiate by depletion of mitogens from the medium. Expression of α-actin, a muscle specific gene, was used as a marker of differentiation. Seven to twelve hours following feeding with 2% horse serum (low mitogen conditions), the cells showed an increase in pRb mRNA. The increase continued over the next 48 hours (Ibid, FIG. 2). The study estimates a 10-fold Rb mRNA induction, an increase which was accompanied by an increase in α-actin expression. In a B cell line, A20 and a pre-B cell line 300-18, the Rb gene is expressed at very low levels compared to actin. In three plasmacytoma lines, representing very late stages of B cell differentiation, Rb mRNA was 8-fold higher. These results are consistent with those of MEL and S2 cells. All cell lines showed an increase in steady-state Rb mRNA in late stages of differentiation, which is maintained in dividing cells. Based on these observations, Coppola, et al., concluded that in all three lineages (erythroid, muscle, and B-cell) differentiation is associated with increased Rb mRNA.

An enriched epithelial cell population from 20-day fetal rat lungs was immortalized with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of 21 h. At the non-permissive temperature (40° C.), doubling time increased to more than 80 h (Levine 1998²⁷⁹, FIG. 4a). 20-3 cells, incubated at the permissive temperature (33° C.) show almost no Rb mRNA while at the non-permissive temperature (40° C.) the cells show a more than 100-fold increase in Rb mRNA (Ibid, FIG. 6b). The increase is significant at 24 h after temperature shift-up and peaks at 48-72 h (Ibid, FIG. 7a). Terminally differentiated and growth arrested alveolar type 1 cells are first observed at day 20-21 of gestation. Prior to this time the lung shows active growth and cell proliferation. Total RNA was isolated from 17-andday fetal lungs and assayed for Rb mRNA. The results show a 2.5-fold increase in Rb mRNA during this period relative to control gene EFTu.

P19 embryonal carcinoma cells were induced to differentiate into neuroectoderm with retinoic acid (RA). Undifferentiated cells show very low levels of Rb mRNA and protein. Twenty-four hours following RA exposure, the cells showed a marked increase in Rb expression with mRNA levels increasing 15-fold by 4-6 days (Slack 1993²⁸⁰, FIG. 2). RAC65 is a mutant clone of P19 cells that fails to differentiate. The cells contain a truncated RARα receptor. Following RA exposure, the cells showed no increase in Rb mRNA (Ibid, FIG. 3). P19 cells transfected with RB-CAT, a reporter gene driven by the Rb promoter, expressed CAT with kinetics similar to the Rb gene (Ibid, FIGS. 5b, 6). The post-mitotic neurons developed in RA-treated cultures contained only the hypophosphorylated form of pRb (Ibid, FIGS. 7, 8). Based on these observations, Slack, et al., concluded that the increased Rb expression associated with cell differentiation appears to result from enhanced transcription.

DS19/Sc9 is a MEL cell line which when treated with in G1, prolonged the next G1 (Richon 1992²⁸¹, FIG. 2A). The cells which emereged from the prolonged G1, progressed through cell cycle for at least another two to five generations (cycle time of 10 to 12 h), and permanently arrested in G1/G0 expressing characteristic of terminal erythroid differentiation. Over 90% of the DS19/Sc9 cells became irreversibly committed to differentiate by 48 h of culture with HMBA. Protein extracts prepared from asynchronous cultures induced with HMBA demonstrated a 2-to 3-fold increase in total amount of pRb. There was no change in proportions of hypo- or hyper-pRb (Ibid, FIG. 4A). An increase in the level of total pRb was detected as early as 24 h after onset of culture with HMBA, and pRb increased as the number of cells recruited to terminal differentiation increased through 100 h of cultured (Ibid, FIG. 4A). HMBA-induced an increase in pRb in all phases of the cell cycle while no change in pRb protein level was detected in DS19/Sc9 cultured without HMBA. The increase in pRb in cells cultured with HMBA was accompanied by an increase in the level of Rb mRNA. A 3.6-fold increase in Rb transcription was observed with no change in mRNA stability. DS19/VCR-C is a vincristine-resistant variant of the parental DS19/Sc9 with an accelerated rate of differentiation. HMBA treatment of DS19/VCR-C showed a more prolonged G1 arrest and a higher percentage of cell committed to terminal differentiation compared to DS19/Sc9. During G₁ arrest, DS19VCR-C also showed more hypo-pRb compared to DS19/Sc9. In HMBA-induced MEL cells, every cell division increased the absolute amount of pRb protein, whereas the degree of phosphorylation continues to fluctuate through cell cycle progression. This increase was accompanied by an increase in mRNA resulting from an increased rate of transcription. Based on these observations, Richon, et al., proposes the following model. An inducer increases Rb transcription resulting in higher hypo- and total-pRb concentration. The increase in hypo-pRb prolongs G1 however, the initial increase in hypo-pRb is most likely not sufficient for permanent G1 arrest. Therefore, cells reenter the cell cycle for a few more generations. While cells continue to divide, the increased rate of transcription results in hypo-pRb accumulation. When a critical hypo-pRb concentration is reached, the cells irreversibly commit to terminal differentiation. This model describes the determination of the commitment to differentiate as a stochastic process with progressive increases in the probability of G1/G0 arrest and differentiation established through successive cell divisions.

Many studies report a relationship between Rb phosphorylation, cell cycle arrest and differentiation. These studies use the different gel mobility of hyper-pRb relative to un/hypo-pRb to show protein phosphorylation or dephosphorylation. Since these studies are interested in the transition between the two states, they do not report changes in total concentration of each form of pRb. Specifically, they do not quantify protein levels with densitometry. However, in some cases, visual inspection of the blots can provide valuable information. Consider the following study. Actively growing LS174T colon cancer cells, which constitutively express pRb, were induced to differentiate with sodium butyrate. Three days following exposure, a lower molecular weight, or unphosphorylated pRb molecule became visible. After the fourth day of treatment, when significant growth inhibition was observed, the unphosphorylated species were predominant (Schwartz 1998²⁸², FIG. 5). A careful inspection of the blots in FIG. 5 suggests that the concentration of hypo-pRb at day 4 (lane 6) is higher than the initial concentration of hyper-pRb (lane 1 and 2). Even if we assume that dephosphorylation of hyper-pRb produces a hypo-pRb species associated with growth arrest (and not protein degradation), the differences in total concentration at day 0 and day 4 indicate a potential need for increased transcription (an increase in mRNA stability, or rate of translation is also possible).

Summary: The transcription of the Rb gene increases with growth arrest and differentiation.

(iii) Microcompetition Increases Probability of Developing Cancer

Rb is a GABP stimulated gene. Microcompetition decreases Rb transcription, which in turn increases the probability of developing cancer.

(b) BRCA1

(i) BRCA1 and Cell Proliferation

Transcriptional or translational inactivation of the BRCA1 gene increases cell proliferation.

Normal mammary ephithelial cells and MCF-7 breast cancer cells were treated with unmodified 18 base deoxyribonucleotides complementary to the BRCA1 translational initition site. The anti-BRCA1 oligonucleotides decreased BRCA1 mRNA by 70-90% compared to control oligonucleotides (Thompson 1995²⁸³, FIG. 6) and the anti-BRCA1 treated cells showed accelerated proliferation rate (Ibid, FIGS. 4a,c).

NIH3T3 cells were transfected with a vector expressing BRCA1 antisense RNA resulting in reduced expression of endogenous BRCA1 protein. The transfected cells, unlike parental and sense transfectants, showed accelerated growth rate, anchorage independent growth and tumorigenicity in nude mice (Rao 1996²⁸⁴, FIG. 4).

Retroviral transfer of wild-type BRCA1 gene to breast and ovarian cancer cell lines inhibited growth in vitro. Transfection of wild-type BRCA1 also inhibited development of MCF-7 tumors in nude mice. Peritoneal treatement with retroviral vector expressing wild-type BRCA1 inhibited tumor growth and increased survival among mice with established MCF-7 tumors (Hold 1996²⁸⁵). A phase I clinical study employing gene transfer of BRCA1 into 12 patients with extensive metastatic cancer showed stable disease for 4-16 weeks in eight patients, tumor reduction in three patients and radiographic shrinkage of measurable disease in one patient (Tait 1997²⁸⁶) Reduced expression of BRCA1 resulted in increased cell proliferation while increased expression of BRCA1 resulted in reduced tumor development.

(ii) BRCA1 in Cancer

(a) Germline Mutations

The majority of familial breast cancer and ovarian cancer cases result from germline mutations in the BRCA1 gene.

(b) Sporadic Breast Cancer

Many studies showed decreased BRCA1 transcription in sporadic breast tumors (Russell 2000²⁸⁷, Rio 1999²⁸⁸, Rice 1998²⁸⁹, Magdinier 1998²⁹⁰, Ozcelik 1998²⁹¹, Thompson 1995, ibid). The decrease intensifies with tumor progression yet the cause of the decreased transcription is unknown. Two possible causes, somatic mutations and promoter methylation, do not seem to provide an explanation. Somatic mutations of the BRCA1 gene are rare in sporadic breast and ovarian tumors (Russell 2000, ibid, R101999, ibid, Futreal 1994²⁹², Merajver 1995²⁹³), and methylation of the BRCA1 promoter was demonstrated in only a small percentage of sporadic breast cancer samples (Catteau 1999²⁹⁴, Magdinier 1998, ibid, Rice 1998, ibid, Dobrovic 1997²⁹⁵). The majority of breast and ovarian tumors show neither somatic mutations nor promoter methylation.

(iii) Microcompetition Increases Probability of Developing Cancer

BRCA1 is a GABP stimulated gene. Microcompetition decreases BRCA1 transcription, which increases the probability of developing breast and ovarian cancer.

(c) Fas

(i) Fas and Cancer

Cell population density is determined by balancing between cell growth and cell death. Programmed cell death, or apoptosis, is the final step in a series of morphological and biochemical events. Fas antigen is a 48-kDA cell surface receptor homologous to the tumor necrosis factor (TNF) family of transmembrane proteins. Fas binding by the Fas ligand, or by antibodies, triggers rapid cell apoptosis.

Fas induced apoptosis was initially identified in the immune system. Ligation of Fas induced apoptosis in activated T cells, B cells, and natural killer cells. In addition, Fas was identified in many epithelial cells. Although the role of Fas in non-lymphoid tissues is not completely understood, maintenance of normal cell turnover and removal of potentially oncogenic cells have been suggested. Consider, as example, the epithelial layer of colonic mucosa. These cells show a rapid rate of cell turnover and high expression of Fas. It is conceivable that the high rate of colonocyte removal is Fas induced.

(a) Germline Mutations

Germline mutations in Fas gene are associated with spontaneous development of plasmacytoid tumors in lpr mice (Davidson 1998²⁹⁶) and neoplasms in two autoimmune lymphoproliferative syndrome (ALPS) patients (Drappa 1996²⁹⁷).

(b) Sporadic Cancers

Many studies showed progressive reduction in Fas expression in many cancers. Consider, Keane, et al, (1996²⁹⁸) results in breast carcinomas, Gratas, et al., (1998²⁹⁹) results in esophageal carcinomas, Strand, et al., (1996³⁰⁰) results in hepatocellular carcinomas, Moller, et al., (1994³⁰¹) results in colon carcinomas and Leithauser, et al., (1993³⁰²) results in lung carcinomas. The reduced Fas expression results from reduced transcription of the Fas gene. Consider the observations in Das, et al., (2000³⁰³) showing reduced Fas transcription in ovarian, cervical and endometrial carcinoma tissues and four ovarian and three cervical carcinoma cell lines. Also consider the results in Butler, et al., (1998³⁰⁴) demonstrating reduced Fas transcription in colon tumors, and in Keane, et al., (1996, ibid) showing reduced Fas mRNA levels in six out of seven breast cancer cell lines. As in the case of the BRCA1 gene, the cause of decreased transcription is unknown. The same two possible causes, somatic mutations and promoter methylation, also fail to exmplain the observed reduction in Fas transcription. Allelic loss or somatic mutations of the Fas gene are rare (Bertoni 2000³⁰⁵, Lee 1999A³⁰⁶, Lee 1999B³⁰⁷, Shin 1999³⁰⁸, Butler 1998, ibid), and no methylation was found in the Fas promoter (Butler 2000³⁰⁹). The majority of carcinomas show no somatic mutations or promoter methylation in the Fas gene.

(ii) Microcompetition Increased Probability of Developing Cancer

Fas is a GABP stimulated gene. Microcompetition decreases Fas transcription leading to an increased probability of developing cancer.

(3) Signaling

(a) ERK Agents Inhibit Proliferation, Stimulate Differentiation

ERK agents phosphorylate GABP, increase Rb, BRAC I and Fas transcription and induce cell cycle arrest and differentiation.

(i) Constitutive Active MAP Kinase Kinase 1 (MEK1)

AU565 breast carcinoma cells were transiently transfected with a constitutively active MEK1 mutant or a control vector. Expression of the consitiutively active MEK1 resulted in a significant increase in ERK activity as determined by the use of an antibody against phosphorylated ERK (Lessor 1998, ibid, FIG. 6A, B). Oil Red O staining was used as a measure of cell differentiation. 53.6% of cells trasfected with the consititutively activated MEK1 vector were Oil Red 0 positive. In contrast, only 20.8% of the cells transfected with the control vector were positive. Based on these observations, Lessor, et al., concluded that constitute activation of the MEK/ERK pathway in AU565 cells is sufficient to mediate differentiation.

(ii) Heregulinβ1 (HRGβ1)

AU565 breast carcinoma cells were treated with 10 ng/ml HRGβ1 for 7 days. The treatment increased ERK activity 4-fold after 10 min. The initial increase dropped to control levels by 15 min. Following the drop, a second sustained increase in activity was observed for 105 min (Lessor 1998, ibid, FIG. 1). HRGβ1 treatment decreased cell number by 56% as compared to non-treated controls (Ibid, FIG. 4). Addition of 0-10 μM PD98059, a specific MEK inhibitor (see above) resulted in a dose-dependent reversal of HRGβ1-induced cell growth arrest (Ibid, FIG. 4). Pretreatment with PD98059 also inhibited HRGβ1-induced differentiation in a dose-dependent manner (Ibid, FIG. 5), with 10 μM PD98059 completely blocking the HRGβ1-induced differentiation. Based on these observations Lessor, et al., concluded that sustained³ activation of the MEK/ERK pathway is both essential and sufficient for HRGβ1-induced differentiation of AU565 cells.

(iii) Phorbol Ester (TPA)

ML-1, human myeloblastic leukemic cells, were treated with 0.3 ng/ml TPA. As a result, ERK2 activity increased with a 6- and 4-fold induction at 1 and 3 h, respectively. Thereafter, the activity decreased to below basal levels (He 1999³¹⁰, FIG. 1A). The time-dependent ERK2 activation was further illustrated by a shift to a slower-migrating form of ERK2, representing the phosphorylated ERK2 (Ibid, FIG. 1B). ML-1 cells treated with 0.3 ng/ml TPA for 3 days, followed by and additional 3 days in culture after removal of TPA, ceased to proliferate and displayed morphological features typical of monocytes/macrophages (Ibid, FIG. 6c). Exposure to PD98059, the MEK inhibitor, led to a 2- and 10-fold reduction in TPA-activated ERK2 activity at 1 and 3 h, respectively (Ibid, FIG. 3). Cells treated simultaneously with 10 μM PD98059 and 0.3 ng/ml TPA continued to proliferate and exhibited morphology of undifferentiated cells (Ibid, FIG. 6A, D). Based on these observations, He, et al., concluded that activation of the MEK/ERK signaling pathway is necessary for TPA-induced mononuclear cell differentiation.

(iv) Transforming Growth Factor-β1 (TGFβ1)

An enriched epithelial cell population from 20-day fetal rat lungs was immortalized with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of 21 h. At the non-permissive temperature (40° C.) doubling time increased to more than 80 h (Levine 1998, ibid, FIG. 4a). The labeling index is a function of [³H]thymidine incorporation in DNA, and therefore correlates with cell replication. Treatment of 20-3 cells with 5 ng/ml TFGβ1 for 72 h decreased the labeling index to 80% at the permissive temperature (33° C.) and to less than 5% at the non-permissive temperature (40° C.) (Ibid, FIG. 5c). Treated cells cultured at the non-permissive temperature for 72 h and then shifted to the permissive temperature for additional 24 h showed an index below 10%. The low labeling index reveals that extensive terminal growth arrest occurred during the non-permissive temperature period. Treatment with the ERK agent TFGβ1 resulted in reduced replication of the epithelial cells in both permissive and non-permissive temperatures. ³ Exposure to low doses of HRGβ1 (0.01 ng/ml) induced a 7-fold transient 5 min peak in ERK activation which dropped to control levels by 90 min. This dose showed no sustained activation (Ibid, FIG. 1). The 0.01 ng/ml HRGβ1 treatement results in cell proliferation.

(4) Carcinogens

(a) Oxidative Stress Increases the Probability of Developing Cancer

Oxidative stress decreases binding of GABP to the N-box, reduces transcription of GABP stimulated genes, and increases transcription of GABP suppressed genes (see microcompetition chapter above). Microcompetition for GABP also decreases binding of GABP to the N-box, which increases the probability of developing cancer (see above). Therefore, oxidative stress also increases the probability of developing cancer. Moreover, oxidative stress increases replication of some GABP viruses; see, for instance, the stimulating effect of oxidative stress on cytomegalovirus (CMV) (Vossen 1997³¹¹, Scholz 1996³¹²), Epstein-Barr virus (EBV) (Ranjan 1998³¹³, Nakamura 1999³¹⁴), and HIV (Allard 1998A³¹⁵, Allard 1998B³¹⁶). If the cell harbors such a GABP virus, the probability of developing cancer as a result of oxidative stress is even higher.

(b) Carcinogens Induce Oxidative Stress

Many carcinogens, genetic and epigenetic, induce oxidative stress, see, for instance, nicotine (Helen 2000³¹⁷, Yildiz 1999³¹⁸, Yildiz 1998³¹⁹) and asbestos (Afaq 2000³²⁰, Abidi 1999³²¹, Liu 2000³²², Marczynski 2000A³²³, Marczynski 2000B³²⁴, Fisher 2000³²⁵, Brown 2000³²⁶). By increasing oxidative stress, these carcinogens reduce GABP binding, decrease expression of Rb, fas and BRCA1, and increase the probability of developing cancer. The effect of these carcinogens on GABP binding might be the main reason for their carcinogenic capacity.

(5) Viruses in Cancer

Many studies report detection of viral genomes in human tumors. The following table summerizes some of these reports.

| Virus | Cancer |
|---|---|
| Epstein-Bar virus (EBV) | Burkitt's lymphoma (BL) |
| | Nasopharyngeal carcinoma (NPC) |
| | Hodgkin's disease |
| | Some T-cell lyphomas |
| | Polymorphic B cell lymphomas |
| | B-cell lymphoproliferation in |
| | immunosuppressed individuals |
| | Breast cancer |
| SV40 | Brain tumors |
| | Osteosacromas |
| | Mesotheliomas |
| HIV | Breast cancer |
| Human T cell lymphotrophic | Adult T-cell leukemia |
| virus-I (HTLV-I) | |
| Human papilloma virus (HPV) | Anogenital cancers |
| | Skin cancers |
| | Oral cancers |
| Hepatitis B virus (HBV) | Hepatocellular carcinoma |
| Hepatitis C virus (HCV) | Hepatocellular carcinoma |
| Human herpes virus 8 | Kaposi's sarcoma, |
| (HHV8, KSHV) | Body cavity lymphoma |

See also recent reviews on human tumor viruses, Butel 2000³²⁷, zur Hausen 1999³²⁸, Hoppe-Seyler 1999³²⁹. On EBV and breast cancer see Bonnet 1999³³⁰, Labrecque 1995³³¹, and the editorial by Magrath and Bhatia 1999³³². On HIV and breast cancer see Rakowicz-Szulczynska 1998³³³.

EBV, SV40, HIV and HTLV-1 are GABP viruses. Microcompetition between a GABP virus and cellular genes causes cancer. An interesting aspect of microcompetition is its ability to explain how viral infection can cause cancer independent of proto-oncogene expression or viral integration into host DNA.

(b) Atherosclerosis

(1) Motility

(a) Introduction

(b) ECM-Cell and Cell-Cell Adhesion

The extracellular matrix (ECM) is comprised of several proteins, including collagens, fibronectin, laminins and proteoglycans assembled into a network structure. Cells bind to ECM proteins through transmembrane-surface receptors. The receptors include integrins, cadherins, immunoglobulins, selectins and proteoglycans. The cadherins and selectins are mostly involved in cell-cell adhesion. The integrins and proteoglycans are mostly involved in cell-ECM binding. Cell-adhesion molecules connect external ligands and the cytoskeleton and participate in signal-transduction.

(c) Motility

A cell is said to show motility if it changes position over time. A change of position of the entire cell is called migration. A change in position of any part of the cell periphery is called projection. The two processes share common features, such as polarization, cytoskeletal reorganization and formation of new cell-ECM adhesion points.

(d) Morphology

The first phase in cell migration is polarization. During polarization the cell creates clear “front-back” asymmetry in which actin and cell-surface receptors accumulate at the leading edge of the cell. The second phase of migration is protrusion of the plasma membrane from the front of the cell in the form of fine, tubular structures called filapodia, or a broad, flat membrane sheet called lamellipodium. The third phase is establishing new ECM-cell points of contact. This binding prevents retraction of the newly extended membrane and provides “grip” for the tractional force required for cell movement. The two final stages of cell migration are flux of intracellular organelles into the newly extended sections of the cell, and retraction of, or breaking off, the trailing edge. The result of this process is directional movement of the cell body (Sanserson 1999³³⁴)

(e) Direction

A simple characterization of direction of movement is a change in distance relative to a reference point in space. Let circulating blood define such a reference point. Movement of cells out, or away from circulation, will be called forward motility. Diapedesis of monocytes to enter the intima (also called migration, emigration or transmigration) is an example of forward motility. Movement of macrophages deeper into the intima is another example of forward motility. Movement of cells toward, or into circulation, will be called backward motility. Reverse transendothelial migration is an example of backward motility.

(2) P-Selectin-, β₂ Integrin-, α₄-Integrin-Propelled Forward Motility

The first section discusses the relationship between p-selectin, β₂ integrin and α₄-integrin and motility without reference to direction. The direction issue is covered in the second section.

(a) Motility

(i) Transendothelial Migration

Leukocyte migration from blood into tissue starts with crossing the endothelium. This phase is called transedothelial migration, transmigration or emigration. Transmigration involves multiple steps, including rolling of leukocytes along the endothelium, firm adhesion of leukocytes to endothelium called margination, and movement of leukocytes through endothelial intercellular junctions. In this process P-selectin mediates rolling of leukocytes on the endothelium (Dore 1993³³⁵). An increase in endothelial surface expression of P-selectin increases leukocyte rolling and transmigration.

Many studies demonstrated the role of the surface receptors CD18 (CD11a/CD18, CD11b/CD18, CD11c/CD18) and VLA-4 (α₄β₁, CD49d/CD29) in this process of transedothelial migration (Shang 1998A³³⁶, Shang 1998B³³⁷, Meerschaert 1995³³⁸, Meerschaert 1994³³⁹, Chuluyan 1993³⁴⁰, Kavanaugh 1991³⁴¹). The two studies by Shang, et al., (1998A, 1998B) also showed that these molecules participate in forward motility through a barrier of human synovial fibroblasts (HSF).

(ii) Intimal Motility

CD18 and α₄ also participate in motility inside the intima. Consider the following studies.

To test the effect of α₄ expression on cell motility, α₄ was expressed in a Chinese hamster ovary (CHO) cell line deficient in α₅β₁ integrin (CHO B2). The parental α₅ deficient CHO B2 cells were unable to adhere, spread or migrate on a surface coated with 10 μg/ml mouse cellular fibronectin. Expression of α₄β₁ integrin in the CHO B2 cells enabled the cells to adhere, spread and migrate on the fibronectin-coated surface (Wu 1995³⁴²).

To test the effect of CD18 on cell motility, neutrophils were stimulated with 0.5×10⁻⁸ M fMLP. The stimulation increased random motility through a three-dimensional collagen type I gel (0.1 to 1.0 mg/mL). In a 0.4-mg/mL collagen gel, antibodies against CD18 (anti-CD18) decreased motility of stimulated neutrophils by 70% (Saltzman 1999³⁴³). Based on these observations Saltzman, et al., concluded that under conditions of high hydration, or when fiber density is relatively low, neutrophil migration through collagen gels is CD18-dependent.

To test the effect of CD18 on cell motility, another study stimulated neutrophils with 10⁻⁸ M FMLP for 10 min. On unstimulated cells, CD18 was randomly distributed on the nonvillous planar cell body. Stimulation of the round, smooth neutrophils induced a front-tail polarity, i.e., a ruffled frontal pole and contracted rear pole with a distinct tail knob at the posterior pole. Moreover, immunogold-labeling and backscattered electron images detected a 4-fold increase in CD18 surface membrane concentration compared to unstimulated cells. The immonogold-labled CD18 accumulated mainly on ruffled plasma membrane at the frontal pole of polar neutrophils. The contracted rear end showed few colloidal gold particles (Fernandez-Segura 1996³⁴⁴). Based on these observations, Fernandez-Segura, et al., concluded that CD18 may participate in the locomotion of neutrophils.

A third study stimulated rat mesentery with platelet-activating factor (PAF; 10⁻⁷ M). After 30-40 min of the chemotactic stimulation, numerous polymorphonuclear leukocytes (PMNs), predominantly neutrophils and monocytes/macrophages, were observed migrating further into the extravascular tissue. Immunofluorescence flow cytometry revealed a 3-fold increase in CD18 expression on extravasated PMNs compared with blood PMNs. Intravital time-lapse videomicroscopy was used to analyze migration velocity of activated PMNs. Median migration velocity in response to PAF stimulation was 15.5±4.5 μm/min (mean±SD). Treatment with two different antibodies against CD18 significantly reduced migration velocity by 17% (mAb CL26) and 22% (mAb WT.3) (Werr 1998³⁴⁵). Based on these in vivo observations Werr, et al., concluded that CD18 participates in extravascular PMN locomotion.

Since the extracellular matrix (ECM) contains fibronection and collagen, the observations of Wu (1995, ibid) and Saltzman (1999, ibid) above are consistent with intimal α₄ integrin- and CD18-propelled leukocyte motility. Moreover, the morphological changes reported by Fernandez-Segura (1996) and the extravascular CD18-propelled leukocyte motility reported by Werr (1998) support such a mechanism.

(b) Direction

The first segment of leukocyte forward motility, transedothelial transmigration, is α₄ integrin- and CD18-propelled. From the basal side of the endothelium, leukocytes continue their forward motility into the intima until they reach a certain depth. Werr, et al., (1998) showed that forward motility in the extravascular space is CD18-propelled. Since the intima is sandwiched between the endothelium and the extravascular space, forward motility in the intimal segment is, most likely, CD18-propelled.

(See more on direction control, or “cell turning,” below)

(3) TF-Propelled Backward Motility

As above, the first section discussed the relation between TF and motility without reference to direction. The direction is covered in the second section.

(a) Motility

TF expression induces cell spreading. Consider the following studies.

The human breast cancer cell line MCF-7 constitutively expresses TF on the cell surface. aMCF-7 is a subline of MCF-7. Muller, et al., (1999, ibid) show that adhesion of aMCF-7 cells to surfaces coated with FVIIa or inactivated FVIIa (DEGR-FVIIa) was significantly accelerated during the first 2 h after seeding compared to surfaces coated with BSA. In addition, the number of cells adhering to anti-TF IgG was significantly higher than the number of cells adhering to anti-FVII or a control IgG (Ibid, FIG. 6A). Accelerated adhesion and spreading of cells on surfaces coated with anti-TF mAb VIC7 was blocked by recombinant TF variants (sTF₁₋₂₁₉, sTF₉₇₋₂₁₉) covering the epitope of anti-TF mAb VIC7 (residues 181-214). No effect was seen with sTF₁₋₁₂₂. However, if anti-TF 111D8 (epitope area 1-25) was used for coating, sTF₁₋₁₂₂ blocked accelerated adhesion and spreading of cells. To conclude, the Muller, et al., results demonstrate that in vitro-cultured cells, that constitutively express TF on the cell surface, adhere and spread on surfaces coated with both catalytically active and inactive immobilized ligands for TF. Ott, et al, (1998³⁴⁶) showed that J82 bladder carcinoma cells that constitutively express high levels of TF adhere and spread on surfaces coated with monoclonal antibodies specific for the extracellular domain of TF. The spontaneously transformed endothelial cell line ECV304 or human HUVEC-C endothelial cells also adhered and spread on TF ligand when stimulated with TNFα to induce TF expression.

In malignant and nonmalignant spreading epithelial cells, TF is localized at the cell surface in close proximity to, or in association with, both actin and actin-binding proteins in lamellipodes and microspikes, at ruffled membrane areas and at leading edges. Cellular TF expressions, at highly dynamic membrane areas, suggest an association between TF and elements of the cytoskeleton (Muller 1999³⁴⁷). Cunningham, et al., (1992³⁴⁸) showed that cells deficient in actin binding protein 280 (ABP-280) have impaired cell motility. Transfection of ABP-280 into these cells restored translocational motility. Ott, et al., (1998, ibid) identified ABP-280 as a ligand for the TF cytoplasmic domain and showed that ligation of the TF extracellular domain by either FVIIa or anti-TF resulted in ligation of the TF cytoplasmic domain by ABP-280, reorganization of the subcortical actin network, and expression of specific adhesion contacts different from integrin mediated focal adhesions.

(b) Direction

(i) Reverse Transendothelial Migration

Randolph, et al., (1998³⁴⁹) used an in vitro model consisting of HUVEC grown on reconstituted bovine type I collagen. The reverse transmigration assays used freshly isolated or precultured peripheral blood monoculear cells (PBMC) incubated with endothelium for 1 or 2 hours to allow accumulation of monocytes in the subendothelial collagen. Following initial incubation, the nonmigrated cells were removed by rinsing the cultures. At given intervals a few cultures were processed to enable counting of the cells underneath the endothelium. The remaining cultures were rinsed to remove cells that may have accumulated in the apical compartment by reverse transmigration, and incubation was continued. Let percent reverse transmigration represent the percentage decrease in the number of cells beneath the endothelium relative to the number of subendothelial cells at 2 hours. FIG. 17 shows the percent reverse transmigration as a function of time.

The results showed that mononuclear phagocytes (MP) that enter the subendothelial collagen later exit the cultures by retransversing the endothelium with a t1/2 of 48 hours. The endothelial monolayer remained intact throughout the experiments.

(ii) Role of Tissue Factor in Reverse Transendothelial Migration

Two MoAbs against TF, VIC7 and HTF-K108, strongly inhibited reverse transmigration for at least 48 hours (Ibid, FIG. 2A). In comparison, 55 other isotype-matched MoAbs tested had little or no effect, specifically, anti-factor VIIa, —IVE4 or —IIH2 did not inhibit reverse transmigration (Ibid, FIG. 2C). A direct comparison of the effect of VIC7 relative to IB4, a MoAb against β2 integrin, revealed 78±15% inhibition of reverse transendothelial migration by VIC7 relative to no inhibition by IB4 in the same three experiments (Ibid, FIG. 2B). None of the MoAbs affected the total number of live cells in the cultures.

(iii) TF Amino Acids 181-214 Essential for Reverse Transmigration

Studies of epitope mapping showed that the epitope for VIC7 included recognition of at least some amino acids between residues 181-214. Soluble TF inhibited reverse transmigration by 69±2% in eight independent experiments (Ibid, FIG. 4). Only fragments containing amino acid residues carboxyl to residue 202 blocked reverse transmigration effectively (Ibid, FIG. 4). This result agrees well with the location of the epitope for VIC7.

(iv) TF and Endothelium Adhesion

Experiments were conducted to explore the existence of a ligand to TF on the endothelium. Unstimulated HUVEC were added to wells coated with TF or control proteins in the presence or absence of anti-TF MoAb. After 2 hours incubation, endothelial cell adhesion to TF fragments containing amino acid residues 202-219 was greater than their binding to control surfaces or to TF fragments lacking these residues (Ibid, FIG. 8A). Spreading of HUVEC during the first 2 hours was observed on surfaces coated with TF fragments carrying residues 97-219 or 1-219. Surfaces coated with a TF fragment spanning amino acids 1-122 showed much less spreading. These results show that endothelial cells express binding sites for TF, and that the TF residues 202-219 participate in this adhesion.

(v) Reverse Transmigration and TF Self Association

LPS stimulation increases cell surface TF activity through increased concentration of cell surface TF molecules and increased conversion of TF dimers to monomers. Monocytes and HUVEC were stimulated with LPS. VIC7 recognized a single band of 47 kD in the LPS-stimulated cells, but not in the unstimulated cell extracts (Ibid, FIG. 3). In unstimulated cells TF is self-associated, most likely in the 181-219 region, and, therefore, unavailable for VIC7 binding. LPS stimulation converts the dimers to monomers and exposes the VIC7 binding site. The same region participates in binding to endothelial cells. Since VIC7 inhibits reverse transmigration by competitive binding to the 181-219 region, self-association also inhibits reverse transmigration.

(4) Cell Turning

Let CD18, α₄ integrin and TF be called propulsion genes. Since leukocyte forward motility is α₄ integrin- and CD18-propelled, and backward motility is TF-propelled, a signaling system should exist that coordinates expression of the proplusion genes. This system should determine the direction of cell motility. The following sections describe such a system.

(a) Two Propulsion Systems

Forward and backward motility are propelled through mostly different molecules.

Antibodies against many molecules participating in forward motility do not inhibit reverse transmigration. Randolph, et al., (1998, ibid) tested a variety of MoAbs against a list of molecules known to mediate binding between leukocytes and endothelium during apical-to-basal transmigration. Even though MoAbs were shown to access subendothelial antigens, neutralizing MoAbs to E-selectin, vascular cell adhesion molecule-1 (VCAM-1), and platelet/endothelial cell adhesion molecule-1 (PECAM-1) showed no effect on reverse transmigration. Ott, et al., (1998, ibid) showed that a RGD peptide known to block several matrix-binding integrins does not abolish spreading on coagulation protease factor VIIa (Ibid, FIG. 2A).

On the other hand, antibodies against TF, which participates in backward motility, do not inhibit forward motility. Resting monocytes do not express TF, however LPS stimulates their expression of TF. Randolph, et al., (1998, ibid) showed that the TF MoAb VIC7 inhibits adhesion of LPS-stimulated, but not resting, monocytes to unstimulated or TNF-activated HUVEC by 35±7%. However, VIC7 did not inhibit migration of LPS-stimulated monocytes already bound to the apical side of the endothelium. Since circulating monocytes do not express TF, it is reasonable to conclude that TF does not participate in adhesion to the endothelium during forward motility (TF adhesion to the apical side of the endothelium is probably important in backward motility, see below). Since TF also does not participate in the subsequent steps in apical-to-basal transendothelial migration, TF has no role in forward motility.

Ott, et al., (1998, ibid) also noted that J82 cells spreading on TF ligand have a different morphology compared to cells adherent to fibronectin through integrins (Ibid, FIGS. 2A and 2B), thereby suggesting a qualitative difference in the two adhesive events.

(b) Signaling

(i) Extracellular Effects on Forward Motility

Extracellular signal-regulated kinase (ERK) agents are extracellular molecules, which transmit a signal resulting in the phosphorylation of ERK. See chapter on ERK for examples. ERK agents stimulate GABP·p300 binding. In leukocytes, this binding stimulates transcription of CD18 and α₄, which, in turn, stimulates forward motility. Moreover, the stimulated binding of GABP·p300 represses TF, and therefore, represses backward motility.

A molecules is regarded a chemoattractant if it stimulates leukocytes forward motility. Considering chemoattraction in the framework of propulsion yields an interesting insight. In leukocytes, chemoattraction is the result of ERK phosphorylation. In other words, if a molecule leads to the phosphorylation of ERK, it should show chemoattraction. fMLP is an example for such a molecule. FMLP is a syntactic compound found in bacterial products. Several studies demonstrated that fMLP binding to its receptor results in phosphorylation of ERK1 and ERK2 (Chang 1999³⁵⁰ in rat neutrophils, Yagisawa 1999³⁵¹ in human monocytes, Coffer 1998³⁵² in human neutrophils). As an ERK agent, fMLP should demonstrate chemoaftraction. As expected, Yamada, et al., (1992³⁵³) showed that fMLP is a chemoattractant for blood mononuclear cells.

Mildly oxidized LDL (also termed “minimally modified” LDL, and therefore denoted mmLDL) and oxidized LDL (oxLDL) are also ERK agents. Consider the following studies.

Rat vascular smooth muscle cells (VSMC) were exposed to 25 μg/ml of Cu⁺²-oxidized LDL (oxLDL). The results showed a rapid stimulation of both ERK1 and ERK2 with peak activity at 5 min and a return to near baseline by 60 min (Kusuhara 1997³⁵⁴, FIG. 1). 25 μg/mL of minimally oxidized LDL (mmLDL) caused a smaller increase in ERK activity with a similar time course (Kusuhara et al., call this type of LDL “native LDL.” However, they propose that this type of LDL is actually minimally oxidized. Therefore, we call it mmLDL). The increase in ERK activity relative to 200 mmol/L PMA treatment was 54.3% for oxLDL and 35.2% for mmLDL. Both oxLDL and mmLDL stimulated ERK activity in a concentration-dependent manner (Ibid, FIG. 3). Human monocytes showed minimal ERK stimulation by either oxLDL or mmLDL (Ibid, FIG. 7A). In contrast, human monocyte-derived macrophages cultured for 7 days showed significant ERK activity in response to oxLDL (Ibid, FIG. 7B) but no response to mmLDL (Ibid, FIG. 7B). Bovine aortic endothelial cells showed no response to either oxLDL or mmLDL (Ibid, FIG. 7C). Based on these observations Kusuhara, et al., concluded ERK activation is cell type dependent, degree of oxidation dependent, LDL receptor dependent and that the rapidity of the ERK response to LDL indicates that ERK activation is LDL internalization independent.

Deigner, et al., (1996³⁵⁵) reported similar effects of mmLDL and oxLDL on ERKin U-937 macrophage-like cells, Balagopalakrishna, et al., (1997³⁵⁶) in aortic smooth muscle cell, Kamanna, et al., (1999³⁵⁷) and Bassa, et al., (1998³⁵⁸) in mesangial cells.

Both mmLDL and oxLDL are ERK agents, and therefore, chemoattractants. Quinn, et al., (1987³⁵⁹) demonstrated that oxLDL is a chemoattractant when bound to macrophages in the subendothelial space. However, in contrast to stimulated macrophages, circulating monocytes are not chemoattracted by oxLDL binding. To chemoattract monocytes, oxLDL uses an indirect approach. Subendothelial oxLDL stimulates endothelial cells to produce monocytes chemoattractant (chemotactic) protein-1 (MCP-1, also called RANTES), which is an ERK agent. MCP-1 is released into circulation and binds monocytes. Monocyte bound MCP-1 stimulates CD18 and α₄ integrin, resulting in adhesion to endothelium and transmigration.

Another special example is baterial LPS, a known chemoattractant which is an ERK agent. LPS is a direct chemoattractant when bound to its receptor (before internalization), and an indirect chemoattractant through stimulation of MCP-1 which is a strong ERK agent.

(ii) Intracellular Effects on Forward and Backward Motility

(a) Redox Regulation of GABP N-Box Binding

Oxidative stress decreases the binding of GABP to the N-box, reduces transcription of GABP stimulated genes and increases transcription of GABP suppressed genes. Consider the following study.

Mouse 3T3 cells were treated for 2 h with diethyl maleate (DEM), a glutathione (GSH)-depleting agent, in the presence or absence of N-acetylcysteine (NAC), an antioxidant and a precursor of GSH synthesis. Following treatment, the cells were harvested, and nuclear extracts were prepared in the absence of a reducing agent. GABP DNA binding activity was measured by EMSA analysis using oligonucleotide probes containing a single N-box (AGGAAG) or two tandem N-boxes (AGGAAGAGGAAG). Treatment of 3T3 cells with DEM resulted in a dramatic decrease in the formation of GABP heterodimer (GABPαGABPβ), (Martin 1996, ibid, FIG. 2A, lane 2) and heterotetramer (GABPα₂GABPβ₂), (Iβιδ, Φιγ. 2A, lane 6) complexes on the single and double N-box. Inhibition of GABP DNA binding activity by DEM treatment was prevented by simultaneous addition of NAC (Ibid, FIG. 2A, lanes 4 and 8). The reduction of GABP DNA binding activity was not due to loss of GABP protein since the amount of GABPα and GABPβ1 was unaffected by DEM or NAC treatment. Dithiothreitol (DTT) is an antioxidant. DTT treatment of nuclear extracts prepared from DEM-treated 3T3 cells restored GABP binding activity. Treatment of 3T3 nuclear extracts with 5 mM GSSG nearly abolished GABP DNA binding. Based on these observations Martin et al., concluded that GABP DNA binding activity is inhibited by oxidative stress, i.e. GSH depletion. The study also measured the effect of DEM treatment on the expression of transiently transfected luciferase reporter constructs containing a TATA box with either an upstream double N-box or C/EBP binding site (Ibid, FIG. 4). DEM treatment had no effect on luciferase expression from C/EBP-TA-Luc after 6 or 8 h treatment (Ibid, FIG. 4). However, DEM treatment of cells transfected with double N-box-TATA-Luc, resulted in a 28% decrease in luciferase expression after 6 h and a 62% decrease after 8 h (Ibid, FIG. 4). Based on these results, Martin et al., concluded that glutathione depletion inhibits GABP DNA binding activity resulting in reduced expression of GABP-regulated genes.

Oxidative stress decreases GABP binding to the N-box, which in turn decreases transcription of a GABP stimulated gene and increases transcription of a GABP repressed gene.

Microcompetition for GABP also decreases binding of GABP to the N-box. Take a GABP regulated gene sensitive to oxidative stress through GABP only⁴. The effect of microcompetition on the transcription of this gene is similar to the effect of oxidative stress. In other words, for this gene, microcompetition can be viewed as leading to “excess oxidative stress.” 4 Oxidative stress also modifies binding of other transcription factors, such as AP1, and NF-κB.

(b) Redox Regulation of Propulsion Genes

Oxidative stress reduces the binding of GABPα to the N-box. Assume the propulsion genes, TF, CD18 and α4 integrin, are responsive to oxidative stress exclusively through GABP. GABP stimulates CD18 and α₄ integrin transcription. Reduced binding of GABPα to DNA decreased CD18 and α₄ integrin transcription resulting in diminished forward motility. On the other hand, GABP represses TF transcription, oxidative stress increases TF transcription, stimulating backward motility.

(i) TF

oxLDL Effect on TF Transcription

oxLDL increases TF transcription. Consider the following studies.

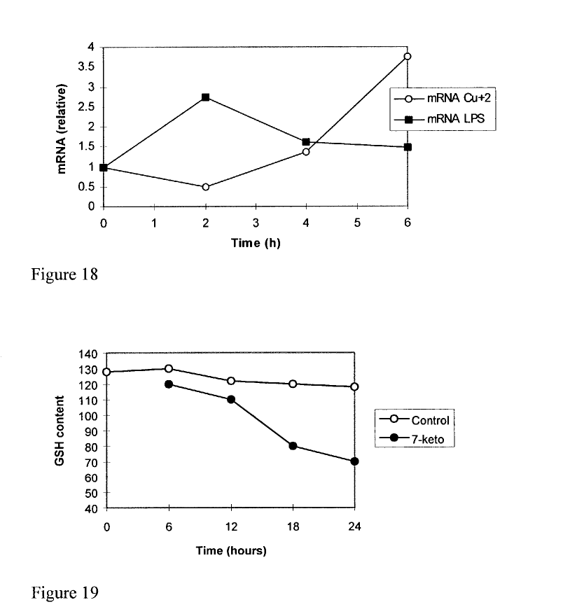

Exposure of human monocytic THP-1 cells for 10 hours to concentrationd of up to 20 μmol/L Cu⁺² had no effect on procoagulant activity. However, in the presence of 1 μmol/L 8-hydroxyquinoline, Cu⁺² produced a dose dependent expression of procoagulant activity (Crutchley 1995³⁶⁰, Table 1). The effect of Cu⁺² was replicated with the copper transporting protein ceruloplasmin. Cu⁺² is known to produce lipid peroxidation and free radical generation. Therefore, the study tested the possibility that the procoagulant activity resulted from oxidative stress. Several lipophilic antioxidants, including probucol (20 μmol/L), vitamin E (50 μmol/L), BHT (50 [mol/L), and a 21-aminosteroid antioxidant U74389G (20 μmol/L), inhibited the Cu+2 induced procoagulant activity (Ibid, FIG. 4). The increased procoagulant activity was due to TF. Cu⁺² induced intracellular oxidative stress, which increased TF transcription. The kinetics of the induction of Cu⁺² was compared to LPS. Exposure to LPS or Cu⁺² resulted in an increase TF mRNA levels. Relative to basal levels, LPS increased mRNA 2.5-fold after 2 hours of exposure, declining to basal levels by 6 hours. In contrast, at 2 hours, Cu⁺² reduced mRNA levels to 50% followed by a 3.5-fold increase at 6 hours (see FIG. 18). The Cu+2 and LPS induced TF expression also differed in the response to antioxidants. While all four antioxidants inhibited Cu+2 induced TF expression, only vitamin E inhibited LPS induced expression.

The LPS effect on TF transcription is mostly mediated through the NF-κB site. Crutchley, et al., (1995, ibid) results indicate that oxidative stress increased TF transcription through a different site. This conclusion is also supported by the negative effect of oxLDL on NF-κB binding to its site as demonstrated in human T-lymphocytes (Caspar 1999³⁶¹), Raw 264.7, a mouse macrophage cell line (Matsumura 1999³⁶²), peritoneal macrophages (Hamilton 1998³⁶³), macrophages (Schackelford 1995³⁶⁴), and human monocyte derived macrophage (Ohlsson 1996³⁶⁵). The results of these studies are consistent with reduced binding of GABP to the N-box in the (−363 to −343) region of the TF gene (see above).

Another study tested the effect of oxLDL on TF transcription. The binding of advanced glycation end products (AGE) with their receptor (RAGE) result in intracellular oxidative stress indicated by reduced glutathione (GSH) levels (Yan 1994³⁶⁶). Monocytes incubated with AGE-albumin (AGE-alb) for 24 hours showed an increase in TF mRNA expression (Khechai 1997³⁶⁷, FIG. 1B). Presence of the translational inhibitor cycloheximide completely suppressed the AGE-alb induced TF mRNA accumulation (Ibid, FIG. 1B). The antioxidant N-Acetylcysteine (NAC) increases the levels GSH and NAC is easily transported into the cell. Incubation of cells with AGE-alb in the presence of 30 mmol/L NAC resulted in a concentration dependent inhibition of TF activity (Ibid, FIG. 2A) and TF antigen expression. Moreover, TF mRNA expression was almost completely suppressed (Ibid, FIG. 2C). Based on these results Khechai, et al., concluded that oxidative stress is responsible for TF gene expression.

Crutchley, et al., (1995, ibid) showed that although reduced oxidative stress decreases TF mRNA, the LPS induced increase in TF mRNA is insensitive to certain antioxidant. Brisseau, et al., (1995³⁶⁸) showed a similar insensitivity of the LPS induced increase in TF mRNA to the antioxidant NAC. Since Khechai, et al., (1997) reported that NAC increases TF mRNA, the combined results of Brisseau, et al., (1995) and Khechai, et al., (1997) are also consistent with reduced GABP binding to the N-box in the (−363 to −343) region resulting from oxidative stress.

See also Ichikawa, et al., (1998³⁶⁹) which reported simliar results in human macrophage-like U937 cells treated with the oxidant AGE and the antioxidants catalase and probucol.

oxLDL Effect on TF Antigen Localization

The induced TF is localized to regions important in cell motility. Consider the following studies.

Endotoxin treatment of human glioblastoma cells (U87MG) resulted in preferential localization of TF antigen in membrane ruffles and peripheral pseudopods. Most prominent TF staining was observed along thin cytoplasmic extensions at the periphery of the cells. Moreover, membrane blebs, associated with cell migration, were also heavily stained (Carson 1993³⁷⁰). Endotoxin treatment of macrophages also resulted in a high concentration of TF antigen in membrane ruffles and microvilli relative to smooth areas of the plasma membrane or endocytosis pits (Lewis 1995³⁷¹, FIG. 2). The membrane ruffles and microvilli contained a delicate, three dimensional network of short fibrin fibers and fibrin protofibrils decorated in a linear fashion with anti-fibrin(ogen) antibodies. oxLDL treatment of macrophages resulted in similar preferential localization of TF antigen in membrane ruffles and microvilli.

Although the two studies use different terms, “cytoplasmic extensions” and “blebbed” (Carson 1993), vs “microvilli” and “membrane ruffles” (Lewis 1995, ibid), the terms, most likely, describe the same phenomenon.

oxLDL Effect on TF Activity

oxLDL increases TF activity. Consider the following study.

Lewis, et al., (1995, ibid) demonstrate the effect of oxLDL treatment on TF activity. In culture, monocytes, and monocyte-derived macrophages expressed little or no procoagulant activity. Endotoxin treatment induced TF activity, peaking at 4 to 6 hours and decreasing over the following 18 hours (Ibid, FIG. 1). Cells exposed to minimally oxidized LDL (oxLDL) showed similar TF activation. The endotoxin and oxLDL treatments resulted in 115- and 58-fold increase in TF activity, respectively (Ibid, Table 1).

oxLDL Effect in Non-Monocytic Cells

oxLDL also increases TF mRNA in smooth muscle cells (SMC) and endothelial cells.

Consider the following two studies.

Quiescent rat SMC contained low levels of TF mRNA. Treatment of SMC with LDL or oxLDL significantly increased TF mRNA (Cui 1999³⁷², FIG. 1). Densitometric analysis showed that oxLDL increases TF mRNA 38% more than does LDL. The accumulation of TF mRNA induced by LDL or oxLDL was transient. Maximum level of TF mRNA was observed 1.5-2 hours following LDL or oxLDL stimulation (Ibid, FIG. 2), declining significantly over the following 5 hours. TF mRNA response to stimulation in human aortic SMC was similar. Nuclear run-on assays and mRNA stability experiments indicated that the increase in TF mRNA resulted mainly from increased transcription.

Another study exposed human endothelial cells to minimally oxidized LDL (oxLDL) or endotoxin for varying times. Northern blot analysis of total RNA showed a sharp increase in TF mRNA at 1 hour, a peak at 2 to 3 hours, and a decline to basal levels at 6 to 8 hours after treatment. The half-life of TF mRNA in oxLDL and endotoxin exposed endothelial cells was approximately 45 and 40 minutes, respectively. The rate of TF mRNA degradation was similar at 1 and 4 hours post treatment. Nuclear runoff assays showed a significant increase in TF transcription rate following exposure of the cells to oxLDL or LPS (Fei 1993³⁷³).

In monocytes/macrophages, oxLDL treatment reduces the binding of NF-κB to its site (see above). Since NF-kB stimulates TF transcription, the decreased binding diminishes the positive oxLDL effect on TF transcription mediated through the GABP site. In endothelial cells (Li 2000³⁷⁴) and smooth muscle cells (Maziere 1996³⁷⁵), oxLDL treatment increases the binding of NF-κB. This increase adds to the positive GABP mediated effect.

(ii) CD18

Oxidative stress reduces CD18 transcription. Consider the following study.

ICAM-1 is a ligand for CD18. Human polymorphonuclear leukocytes (PMN) were exposed to hypoxic condition. As a result, the adhesion of PMN to recombinant ICAM-1, but not BSA coated surfaces, increased (Montoya 1997³⁷⁶, table 1). Anti-CD18 mAb abolished the increased adhesion (Ibid, FIG. 1). The antioxidant pyrrolidine dithiocarbamate (PDTC) reduced PMN intracellular oxidative stress (Ibid, FIG. 2). PDTC treatment of PMN increased PMN adhesion to tumor necrosis factor-α (TNFα) stimulated HUVEC monolayers (Ibid, FIG. 4). Pyrrolidine, which lacks antioxidant activity, failed to increase adhesion. Anti-CD18 abrogated the PDTC enhanced adhesion (Ibid, FIG. 5). Under flow conditions, a significant number of PMN were rolling at low velocities on the apical surface of the HUVEC monolayer. PDTC treatment reduced rolling distance and rolling velocities (Ibid, FIG. 10), increasing the number of stably adhered PMN. These observations indicate that reduced oxidative stress stimulates CD18 expression.

Hypoxia results in reduced oxidative stress, and therefore, stimulates GABP binding (Martin 1996, ibid). Increased GABP binding stimulates CD18 transcription (Rosmarin 1998, ibid), and therefore, CD18 adhesion. The observations in Montoya (1997, above) are consistent with such a mechanism.

(c) Special Oxidative Stress Inducers

(i) Oxidized LDL

Oxidative stress inducers of special importance (see below) are mmLDL and oxLDL.

Oxidized LDL Depletes GSH

mmLDL and oxLDL deplete intracellular GSH, and therefore induce oxidative stress. Consider the following studies.

GSH content was determined in cultured human endothelial cells after 24 h incubation with native LDL or oxLDL at 30, 40 or 50 μg of protein/ml. The results showed that at 30 μg/mg, GSH content slightly but significantly increased (10%). In contrast, at 40 and 50 μg/ml, GSH content decreased by 15 and 32%, respectively (only significant at 50 μg/ml, P<0.05) (Therond 2000³⁷⁷, FIG. 2B). Moreover, the results also showed that all oxLDL lipid fractions induced depletion of intracellular GSH (Ibid, FIG. 3B).

Another study tested the effect of a specific oxLDL fraction on intracellular GSH. Human promyelocytic leukemia cells U937 were treated with 7-ketocholesterol. U937 cells were used since they respond to oxysterols in concentrations similar to those observed in endothelial and smooth muscle cells, and since U937 are frequently used to model the response of macrophages to oxysterols in humans. The GSH content was measured by flow cytometry with monochlorobimane. The results are summarized in FIG. 19 (Lizard 1998³⁷⁸, FIG. 5A).

At all time points, GSH content in the 7-ketocholesterol treated cells was lower compared to controls (P<0.05).

Oxidized LDL Cell Loading Reduces CD18 Expression

According to Gray and Shankar (1995³⁷⁹) “AthMØ (Atherosclerotic Macrophages) showed a substantial reduction in CD11b and CD18 cell surface expression. NMØ (Normal rabbit peripheral blood Monocytes), on the other hand, had strong surface expression of both CD11b and CD18 . . . . In comparison to NMØ that have been in cell culture for a short time, cell surface expression of the CD11b/CD18 integrin on AthMØ is strongly down-regulated . . . . Furthermore, these immunohistochemical studies provided evidence that the loss of CD11b/CD18 integrins is a function of the extent of lipid loading and perhaps the stage of the foam cell formation . . . . It is our observation from looking at these cytologic preparations, that when stained for adhesion molecules, the smaller more normal appearing cells with very little lipid in them actually have the majority of staining, whereas the larger, more lipid laden cells have absolutely no staining in them.”

Oxidized LDL Cell Loading Reduces Forward Motility

Mouse pertioneal macrophages were loaded with lipids by precincubation with acetylated LDL (acLDL) for various periods (100 μg/ml). The macrophages turned foam cells were used to fill the upper wells of a modified Boyden chamber. The lower wells contained Zymosan A activated mouse serum (ZAMS). Zymosan A is a cell-wall extract of Saccharomyces cerevisiae. ZAMS is a chemoattractant for macrophages. After 3 h, the membrane in the Boyden chamber was removed and the cells, which did not migrate to the lower surface, were wiped off. The migrated cells were fixed and counted. The results showed decreased macrophage migration with increased preincubation time with acLDL. Since, preincubation time correlated positively with lipid content, higher lipid content resulted in reduced migration (Trach 1996³⁸⁰, FIGS. 4a,b). (Similar results are reported in Pataki, et al., (1992³⁸¹), an earlier study with H. Robenek as principle investigator.) Quinn, et al., (1985³⁸²) also reports reduced motility of resident macrophages with modified LDL as chemoattractant.

Bacterial particles are macrophage chemoattractants (for LPS, see above, for fMLP, see Yamada, et al., (1992, ibid)). However, it seems likely that macrophage loading with one type of toxic substance (oxLDL, bacterial particles) reduces chemoattractance of the other. The results in the above studies are consistent with such a concept. In these studies, the zamosan chemoattractance was reduced with the increase in cell loading of modified LDL.

(ii) Bacterial Particles

Bacterial particles, such as LPS or fMLP (a syntatic particle that represents bacterial products), are another important type of oxidative stress inducers (see below).

The products of the respiratory burst have low molecular weight, and therefore, diffuse out of the phagolysosome into cytoplasm and nucleus. The resulting oxidative stress effects TF transcription through the N-box and not the NF-κB site (see above). On the other hand, the bacterial particles, such as LPS, also increase TF transcritpion through the NF-κB site. These two effect act synergistic. Such a synergy is probably needed for quick removal of the relatively highly toxic bacterial particles (compared to oxLDL toxicity) by faster clearance of bacterially loaded macrophages from infected tissues.

(c) Net Propulsion

Consider a tissue resident molecule, which is both an oxidant and an ERK agent. As an ERK agent the molecule chemoattracts circulating or resident leukocytes by increasing their expression of CD18 and α₄ integrin, inducing forward motility. The leukocyte migrate toward the molecule and phagocytize it. Once internalized, the molecule induces oxidative stress, i.e., depletes GSH, which, in turn, reduces binding of GABP to the N-boxes on TF, CD18 and α₄ integrin, resulting in increased expression of TF and reduced expression of CD18 and α₄ integrin. These changes reduce forward propulsion and increase backward propulsion, until backward propulsion is greater. Since net force is the vector sum of all forces acting upon an object, the new net propulsion turns the leukocyte back toward circulation. The final step of this process is reentry into circulation.

(5) Atherosclerosis-Fibrous Cap Atheroma Formation

The first major class of atherosclerotic lesions is the fibrous cap atheroma. The fibrous cap is a distinct layer of connective tissue completely covering a lipid core. The fibrous cap consists of smooth muscle cells in a collagen-proteoglycan matrix with a variable number of macrophages and lymphocytes (Virmani 2000³⁸³). The following sections describe the mechanism of fibrous cap atheroma formation.

(a) LDL Pollution

Plasma LDLs passively cross the endothelium (see below) by diffusion through the plasma membrane. Higher concentration of plasma LDL result in increased influx of LDL. Unlike other tissues, the intima lacks lymphatic vessels. Therefore, to reach the nearest lymphatic vessels, located in the medial layer, the LDL should pass through the intima. However, this passage is partly blocked by an elastic layer situated between the intima and the media (Pentikainen 2000³⁸⁴). According to Nordestgaard, et al., (1990³⁸⁵) “less than 15% of the LDL cholesteryl ester that entered the arterial intima penetrated beyond the internal elastic lamina.” A fraction of the influxed LDL is passively effluxed through the endothelium. Another fraction is hydrolyzed. The remaining intimal LDLs bind the extracelluar matrix (ECM). The ECM is composed of a tight negatively charged proteoglycan network. Certain sequences in the LDL apoB-100 contain clusters of the positively charged amino acids lysine and arginine. These sequences, called heparin-binding domains, interact with the negatively charged sulphate groups of the glycosaminoglycan chains of the proteoglycans (Boren 1998³⁸⁶, Pentikainen 2000, ibid). Subendothelial agents modify (oxidize) the matrix bound LDL.

Passive Influx

Nordestgaard 1992³⁸⁷ reports a linear correlation between plasma concentration of cholesterol in LDL, IDL, VLDL and arterial influx. Moreover, in cholesterol-fed rabbits, pigs and humans, arterial influx of lipoproteins depended on lipoprotein particle size. Other studies report that arterial influx of LDL in normal rabbits did not depend on endothelial LDL receptors. According to Nordestgaard, et al., these results indicate that the transfer of lipoprotein across endothelial cells and into the intima is a “nonspecific molecular sieving mechanism.” Schwenke (1997³⁸⁸) measured the intima-media permeability to LDL in different arterial regions in normal rabbits on a cholesterol-free chow diet. The results showed that the aortic arch is 2.5-fold more permeable to LDL compared to descending thoracic aorta (Ibid, Table 2). The concentration of undegraded LDL in the aortic arch was almost twice as great compared to the descending thoracic aorta (Ibid, Table 3). In cholesterol-fed rabbits, as a result of hypercholesterolemia, the mass transport of LDL cholesterol into all arterial regions was greatly increased. However, hypercholesterolemia did not influence intima-media permeability of any arterial region (Ibid, Table 2). Kao, et al., (1994³⁸⁹), Kao, et al., (1995³⁹⁰) showed that open junctions with gap widths of 30-450 nm between adjacent endothelial cells were only observed in the branched regions of the aortic arch, and not in the unbranched regions of the thoracic aorta. Moreover, LDL labeled with colloidal gold were present within most of these open junctions, while no gold particles were found in the normal intercellular channels (i.e., 25 nm and less) of both regions. These results are consistent with a nonspecific molecular sieving mechanism.

Passive Efflux

Rabbits of the St Thomas's Hospital strain show elevated plasma levels of VLDL, IDL, and LDL. In both lesioned and nonlesioned aortic arches of these rabbits, the logarithms of the fractional loss of VLDL, IDL, LDL, HDL, were inversely and linearly correlated with the diameter of these macromolecules (Nordestgaard 1995³⁹¹). This observation suggests that, similar to influx, the efflux of LDL through the endothelium can also be described as a “nonspecific molecular sieving mechanism.”

(b) LDL Clearance

(i) Model

Modified LDL is chemotactic to circulating monocytes (see above). As a result, endothelial cells increase the surface expression of P-selectin and circulating monocytes increase CD18 and 4 integrin expression (other surface molecules also change their expression). The increased expression of forward propulsion genes increases adhesion of circulating monocytes to the endothelium (margination) and emigration (see forward motility above). Once in the intima, monocytes differentiate into macrophages and start to accumulate modified LDL therby turning into foam cells. The intracellular oxidative stress induced by the modified LDL particles decreases CD18 and a₄ integrin transcription and stimulates TF transcription. The decreased CD18 and α₄ integrin expression reduces forward propulsion. The transient increase in TF activity on the surface of foam cells induces backward propulsion. When backward propulsion surpasses forward propulsion the cell turns back. When the foam cells reach the endothelium, they first bind the basal surface and then the apical surface of the endothelium. When TF adhesion activity returns to its basal level, the apical bound foam cells are released into circulation.

(ii) Observations

(a) Enhanced Forward Motility

There is extensive research showing more adhesion and emigration of monoctyes in atherosclerosis.

(b) Enhanced Backward Motility

(i) Foam Cell Clearance

The results of the following studies are consistent with clearance of foam cells.

Twenty-two Yorkshire pigs were fed a high fat diet. The animals were killed 12, 15 and 30 weeks after diet initiation, and tissue samples were examined by light and electron microscopy. At 15 weeks, lesions were visible as raised ridges even at low magnification (Gerrity 1981³⁹², FIG. 1). Large numbers of monocytes were adherent to the endothelium over lesions, generally in groups (Ibid, FIG. 5), unlike the diffused adhesion observed at prelesion areas. Foam cells overlaid lesions at all three stages, although more frequently at 12 and 15 weeks. The foam cells had numerous flaplike lamellipodia and globular substructure (Ibid, FIG. 6). Some foam cells were fixed while passing through the endothelium, trapped in endothelial junctions alone (Ibid, FIG. 8) or in pairs (Ibid, FIG. 9). In all cases, the attenuated endothelial cells were pushed luminally (Ibid, FIG. 14). The lumenal portion of the trapped foam cells had an irregular shape, with numerous cytoplasmic flaps (lamellipodia and veil structures), empty vacuoles and reduced lipid content compared to the intimal part of the cell (Ibid, FIGS. 8 and 9). Foam cells were also infrequently found in buffy coat preparations from arterial blood samples (Ibid, FIG. 7), and rarely in venous blood. According to Gerrity, these findings are consistent with backward migration of foam cells and suggest that such a migration indicates the existence of a foam cell mediated lipid clearance system.

Another study fed 10 male pigtail monkeys an atherogenic diet and 4 monkeys a control diet. Twelve days after diet initiation, and at monthly intervals up to 13 months, animals were killed and tissue samples were examined by light and electron microscopy. The endothelial surface of the aorta in control animals was covered with a smooth, structurally intact endothelium (Faggiotto 1984-I³⁹³, FIG. 4A). Occasionally, the surface showed small focal areas protruding into the lumen (Ibid, FIG. 4B). Cross sectional examination of the protrusions revealed foam cells underlying the intact endothelium (Ibid, FIG. 3A). During the first 3 months, the endothelium remained intact. However, on larger protrusions the endothelium was extremely thin and highly deformed. At 3 months, the arterial surface contained focal sites of endothelial separation with a foam cell filling the gap (Ibid, FIG. 10A). The luminal section of the foam cell showed numerous lamellipodia. In addition, thin sections of endothelial cells bridged over the exposed foam cell, deforming the surface of the foam cell (Ibid, FIG. 10B). Moreover, rare occasional foam cells were observed in blood smears of some controls. During the first 3 months, when the endothelium was intact, the number of circulating foam cells increased (Faggiotto 1984-II³⁹⁴, FIG. 10). Based on these observations Faggiotto, et al., concluded that foam cells egress from the artery wall into the blood stream, confirming the conclusions of Gerrity (1981).

A third study feed 36 male New Zealand White rabbits a cholesterol-enriched diet and 37 rabbits a control diet. Both groups were exposed to electrical stimulation (ES) known to induce arteriosclerotic lesions. The stimulation program lasted 1, 2, 3, 7, 14, or 28 days. At these intervals, tissue samples were collected, processed, and examined by transmission electron microscopy (TEM). After 1 day of ES, intimal macrophages of hypercholesterolemic rabbits showed loading of lipids (Kling 1993³⁹⁵, FIG. 3b). These cells were often responsible for markedly stretching the overlying endothelial cells. After 2 days, foam cells were fixed while passing through endothelial junctions (Ibid, FIG. 8a). Neighboring endothelial cells were often pushed luminally, indicating outward movement of the macrophage (Ibid, FIG. 8a). The outward movement of the cells was also supported by the finding that the intimal portion of the foam cells transmigrating the endothelium was intact, while the lumina portion was often ruptured and associated with platelets.(Ibid, FIGS. 8b,c). Under the prolonged influence of the atherogenic diet, emerging foam cells became more frequent. In all cases, the emerging foam cells migrated through endothelial junctions without damaging the endothelium. Based on these observations, Kling, et al., concluded that “similar to observations of Gerrity and Faggiotto, et al., we have electro microscopic evidence that the macrophages, loaded with lipid droplets, were capable of migrating back from the intima into the blood stream . . . thus ferrying lipid out of the vessel wall.”

(ii) Increased TF Expression on Foam Cell

The following studies show increased TF expression on foam cells.

Seven White Carneau pigeons were fed an atherogenic diet and three animals received a control diet. The diet regimen lasted 8-10 months and was shown to be sufficient to induce lesions in the thoracic aorta. The concentrations of tissue factor (TF) antigen in circulating monocytes, cultured macrophages, and macrophages from atherosclerotic lesions were ultrastructurally analyzed using immunogold labeling. The plasma cholesterol of the cholesterol-fed animals was elevated compared to controls. Upon dissection, all cholesterol-fed animals revealed fatty streaks and atherosclerotic plaque at the celiac bifurcation of the thoracic aorta. Monocytes isolated from normocholesterolemic and hypercholesterolemic animals had approximately 1 immunogold particle per 2 μm of plasma membrane (Landers 1994³⁹⁶, FIG. 2). The low level of TF antigen in the plasma membrane is consistent with the lack of TF procoagulant activity in freshly isolated monocytes or monocyte-derived macrophages maintained in culture. Monocytes newly adherent to lesion surface also showed low levels of TF antigen (0.3 particles/μm of plasma membrane). In contrast, the lumenally exposed surface of foam cells projecting into the arterial lumen from subendothelial intima showed high levels of TF antigen (7.3 particles/μm of plasma membrane). The distribution of TF concentrations on the surface of macrophages was bimodal. Circulating and newly adherent macrophages had low levels of TF antigen. Projecting foam cells had high level of TF antigen. (The immunogold labeling of endothelial cells either underlying the adherent macrophages or flanking intimal foam cells protruding into the lumen was minimal.) According to Landers, et al., these observations are consistent with the egressing foam cells reported by Gerrity. Another unpublished observation reported in Landers, et al., (1994) is the association between short-term lesion regression and the transient increase in clot formation on lesions.

Faggiotto 1984-I (ibid) showed the existence of foam cells in peripheral blood smears from hyperlipidemic monkeys. Most of these cells showed no adherence to plastic cell culture dishes, however TF induces such adherence. Since egressing foam cells show high concentrations of TF antigen, either TF is removed from the cell surface while in circulation or, more likely, TF adhesion activity is reduced by encryption (see below).

Lander, et al., (1994, ibid) and Faggiotto, et al., (1984, ibid) observations are consistent with the following model. Modified LDL increases TF transcription. The initial increase in TF concentration on the surface of foam cells results in backward motility. The cells pass through gap junctions by first binding the basal and then the apical side of the endothelium. Concurrently, the concentration of surface TF continues to increase. The additional surface TF deactivates many surface TF molecules through the formation of TF dimers (encryption). The encrypted foam cells are consequently released from the endothelium surface and join circulation.

(c) Atherogenesis

(i) Model

Let Trapped_{FC}, Egress_{FC} and Total_{FC} denote the number foam cells trapped in the intima, the number of foam cells in the process of egressing from the subendothelial space and the total number of intimal foam cells, respectively. Trapped_{FC}+Egress_{FC}=Total_{FC}. Denote the fraction of foam cells trapped in the intima with %_{Trapped}. Assume that inefficiencies in foam cell backward motility, denoted I, increase %_{Trapped}, which is the percentage of trapped foam cells. Also assume that %_{Trapped} is independent of Total_{FC}, the total number of intimal foam cells.

Trapped_{FC}=%_{Trapped}(I)×Total_{FC}. (1)

Let Rateₗₑₛᵢₒₙₛ denote the rate of athersclerotic lesion formation.

Rateₗₑₛᵢₒₙₛ=f(Trapped_{FC})=f(%_{Trapped}(I)×Total_{FC}). (2)

The following derivatives summarize the relationship between changes in Total_{FC} or I and Rateₗₑₛᵢₒₙₛ.

$$\begin{matrix}\frac{\partial R a t {e}_{l e s i o n s}}{\partial T o t a {l}_{F C}} = \frac{\partial R a t {e}_{l e s i o n s}}{\partial T r a pp e {d}_{F C}} \cdot {\%}_{T r app e d} & ( 3 ) \\ \frac{\partial R a t {e}_{l e s i o n s}}{\partial I} = \frac{\partial R a t {e}_{l e s i o n s}}{\partial T r a pp e {d}_{F C}} \cdot T o t a {l}_{F C} \cdot \frac{\partial {\%}_{T r app e d}}{\partial I} & ( 4 )\end{matrix}$$

Consider equation 3.

$$\frac{\partial R a t {e}_{l e s i o n s}}{\partial T r a pp e {d}_{F C}} > 0.$$

%_{Trapped} is fixed. Therefore,

$$\frac{\partial {\%}_{T r app e d}}{\partial T o t a {l}_{F C}} > 0 ,$$

an increase in total number of intimal foam cells increases the rate of lesions formation. An increase in LDL pollution increases the entry of monocytes, which increases the total number of intimal foam cells thereby resulting in increased rate of lesion formation.

Consider equation 4.

$$\frac{\partial R a t {e}_{l e s i o n s}}{\partial T r a pp e {d}_{F C}} > 0. T o t a {l}_{F C} > 0. \frac{\partial {\%}_{T r app e d}}{\partial I} > 0.$$

Therefore,

$$\frac{\partial R a t {e}_{l e s i o n s}}{\partial I} > 0 ,$$

an increase in backward motility inefficiencies increases the rate of lesion formation.

(ii) Observations

There are numerous observations consistent with such a model of atherogenesis. Most of these observations relate to the effect of the total number of intimal foam cells on rate of lesion formation (equation 3). For instance, diet or genetically induced hypercholesterolemia increase plasma concentrations of LDL, resulting in increased LDL pollution. The increased oxLDL bound to the ECM chemoattracts monocytes. As expected in equation 3, the increase in Total_{FC} results in an increased rate of lesion formation. Another example is LDL pollution of the edges of blood vessel bifurcations resulting from low shear stress (Malek 1999³⁹⁷). As expected, these areas show a higher propensity to develop atherosclerotic lesions.

The opposite direction also holds. A reduction in LDL pollution reduces the rate of atherosclerotic formation. For instance, studies showed that in an animal, several months of a lipid-reduced diet resulted in a decreased number of foam cells and regression of fatty streaks (Trach 1996, ibid, Pataki 1992, ibid, Wissler 1990³⁹⁸, Dudrick 1987³⁹⁹, Tucker 1971⁴⁰⁰). Other studies showed that a genetic deficiency in ICAM-1, P-selectin or E-selectin (Collins 2000⁴⁰¹), a genetic double deficiency in P-selectin and E-selectin (Dong 1998⁴⁰²) or treatment with monoclonal antibodies against VAL4 or ICAM-1 (Patel 1997⁴⁰³) reduced monocyte recruitment resulting in a diminished rate of atherosclerotic lesions formation. More studies showed that a mutation in all basic amino acids in the proteoglycan-binding region of apoB-100, which prevents binding of the heparin proteoglycans in ECM, resulted in only mild atherosclerosis despite strong hypercholesterolemia (Pentikainen 2000, ibid). The diminished concentration of ECM bound oxLDL attracted fewer monocytes resulting in reduced Total_{FC}.

For a review of the different theories of atherosclerosis, see Stary, et al., (1994⁴⁰⁴).

(iii) Microcompetition

(a) Endothelial Layer

(i) Microcompetition Increased Monocyte Recruitment

Latent infection of endothelial cells increases P-selectin expression thereby inducing increased transmigration of monocytes. According to equation (3) above, the increased number of foam cells increases the rate of lesion formation.

(b) Subendothelial Space

(i) Subendothelial Environment Intensifies Microcompetition

The subendothelial environment transactivates latent viral infection in monocytes turned macrophages. Consider the following studies. Cytomegalovirus (CMV) is a GABP virus. Circulating monocytes are nonpermissive for CMV replication. They show no expression of viral gene products even when cells harbor a viral genome (Taylor-Wiedeman 1994⁴⁰⁵). In monocytes the virus is in a latent state. Viral replication is dependent on expression of viral immediate-early (IE) gene products controlled by the major immediate-early promoter (MIEP). HL-60, promyelocytic leukemia cells that can differentiate into macrophages, were transfected with MIEP-CAT, a reporter-plasmid construct controlled by the CMV MIEP. Coculture of MIEP-CAT-transfected cells with endothelial cells (ECs) increased MIEP-CAT activity 1.7 fold over baseline activity in noncocultured HL-60 cells (Guetta 1997⁴⁶, FIG. 1A). Coculture of MIEP-CAT-transfected cells with smooth muscle cells (SMCs) increased MIEP-CAT activity 4.5-fold over baseline (Ibid, FIG. 1B). Treatment with 50 to 200 μg/mL oxLDL activated MIEP in a concentration dependent manner (Ibid, FIG. 2.). A 2.0-fold increase was the largest observed effect of oxLDL (Ibid, FIG. 1C). Coculture with ECs plus oxLDL led to a 7. 1-fold increase over baseline, larger than the two separate effects. Based on these results Guetta, et al., concluded that exposure of monocytes turned macrophages to ECs, SMCs, and oxLDL in the subendothelial space favors transactivation of latent CMV.

Moreover, when cerulenin, an inhibitor of fatty acid biosynthesis, was added to mouse fibroblasts infected with Moloney murine leukemia virus (MMuLV), virus production was drastically reduced (Ikuta 1986B⁴⁰⁷, Katoh 1986⁴⁰⁸). Cerulenin also inhibited Rous sarcoma virus (RSV) production in chick embryo fibroblasts (Goldfine 1978⁴⁰⁹).

Following entry to the subendothelial space, monocytes differentiate into macrophages. Monocyte differentiation transactivated the human CMV IE gene (Taylor-Wiedeman 1994, ibid), and, in some cases, produced productive HCMV infection (Ibanez 1991⁴¹⁰, Lathey 1991⁴¹¹). Similarly, differentiation of THP-1 premonocytes (Weinshenker 1988⁴¹²) and T2 teratocarcinoma cells (Gonczol 1984⁴¹³) also produced HCMV replication.

Subendothelial monocyte-derived macrophages are exposed to ECs, SMCs and oxLDL. If a macrophage harbors a GABP viral genome, the subendothelial environment stimulates viral replication and the increase in viral DNA intensifies microcompetition.

(ii) Superficial Stop

Increased viral replication in the subendothelial space intensifies microcompetition leading to reduced expression of CD18 and α₄ integrin, which stops the macrophage at a reduced intimal depth. The oxLDL deep in the intima is not cleared and remains ECM bound. While trapped foam cells form fatty streaks, the ECM bound oxLDLs form the lipid core of the atherosclerotic plaque. The following observations are consistent with such a mechanism.

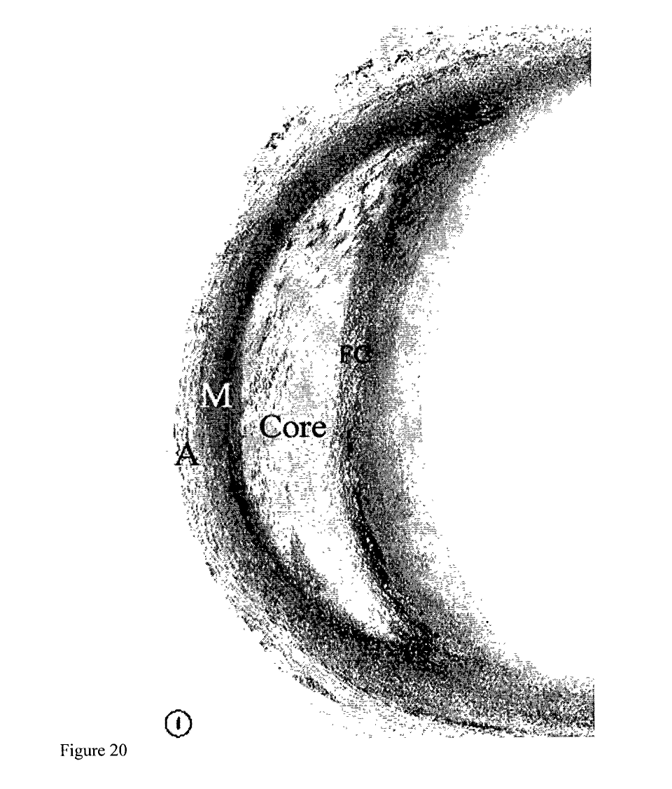

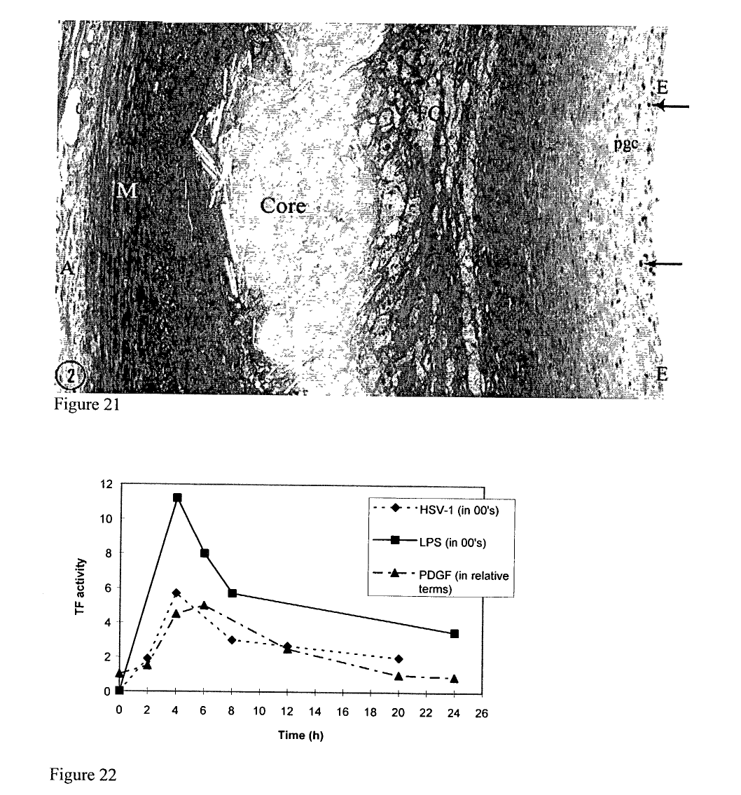

The core of an atherosclerotic plaque actually forms concurrently with fatty streaks. The core has a tendency to extend from a position initially deep in the intima toward the lumen of the artery with increasing age. The lipid in the core region seems to originate directly from plasma lipoproteins and not from foam cell necrosis. Foam cells are usually seen in superficial intima in the region between the core and the endothelial surface (Guyton 1995⁴¹⁴). Consider the following two photomicrographs, FIGS. 20 and 21, as examples (Stary 1995⁴¹⁵, FIG. 1 and FIG. 2).

FIG. 20 is a photomicrograph of atheroma (type IV lesion) in proximal left anterior descending coronary artery from a 23-year old man who died of a homicide. Extracellular lipids form a confluent core in the musculoelastic layer of eccentric adaptive thickening. The region between the core and the endothelial surface contains macrophages and foam cells (FC). There is no increase in smooth muscle cells or collagenous fibers. “A” indicates adventitia, “M,” media. Fixation was performed by pressure-perfusion with glutaraldehyde and maraglas embedding. The sections are one-micron thick. Magnification is about 55×.

FIG. 21 is a photomicrograph of thick part of atheroma (type IV lesion) in proximal left anterior descending coronary artery from a 19-year-old man who committed suicide. Core of extracellular lipid includes the formation of cholesterol crystals. Foam cells (FC) overlie core on the aspect toward lumen. Macrophages that are not foam cells (arrows) occupy the proteoglycan layer (pgc) adjacent to endothelium (E) at lesion surface. “A” indicates adventitia, “M,” media. Fixation was performed by pressure-perfusion with glutaraldehyde and maraglas embedding. The sections are one-micron thick. Magnification is about 220×.

(iii) Reduced Backward Motility

The studies by Randolph, et al., (1996⁴¹⁶) and Randolph, et al., (1998, ibid) (see above) have a similar experimental setting. However, Randolph, et al., (1996, ibid) tested the effect of mAb against ICAM-1 and mAb against CD18 on reverse transmigration. The results showed that Fab fragments of mAb against ICAM-1 (R6.5) completely blocked egression of mononuclear phagocytes (MP) from IL-1-treated HUVEC/amnion cultures for a total of 5 h (Ibid, FIG. 9A). When incubation of MP-HUVEC cocultures (IL-1-pretreated HUVEC) was extended to 12 h, anti-ICAM-1 Fab fragments inhibited reverse transmigration of monocytes by 53% (Ibid, FIG. 9b). Anti CD18 Fab fragments (TS1/18) suppressed reverse transmigration by an average of 71% at 5 h of incubation (Ibid, FIG. 9a). Based on these observations Randolph, et al., concluded that one role of CD18 and ICAM-1 in reverse transmigation is to accelerate initial kinetics.

These results indicate the existence of an intial delay in the activation of TF propelled backward motility. This delay might be necessary to allow other cell changes required for TF propelled motility such as cell skeleton modifications. During this delay other molecules, such as CD18, propel backward motility.

Many studies measured the effect of certain agents on TF activity over the first few hours following treatment. For instance, Key, et al., (1993, ibid) infected HUVEC with herpes simplex virus-1 (HSV-1) or exposed the cells to LPS and measured TF PCA activity. Schecter, et al., (1997, ibid) measured the effect of platelet-derived growth factor (PDGF) stimulation on TF activity on surface of human aortic smooth muscle cell (SMC). The results reported in these studies are presented in FIG. 22. HSV-1 and LPS lines represent PCA activity in U/ml (Key 1993, ibid, FIG. 1). PDGF line represents TF activity relative to untreated cells (Schecter 1997, ibid, FIG. 7)

Lewis, et al., (1995, ibid) reported stimulated monocytes, and monocyte-derived macrophages with oxLDL or LPS (see above) and measured TF activity. The results showed that both agents had similar effects.

Combining the observations in Randolph, et al., (1996, ibid) with these observations suggests that TF driven backward motility starts around the time when TF activity is maximized. Moreover, TF propelled reverse transmigration occurs while TF activity is declining. We call this observation a “soft landing.” We propose that a soft landing might reduce the probability of an undesired coagulation reaction on the surface of egressed foam cells or might increase the probability of foam cell release from the apical surface of endothelium.

In general, ₐTF denotes TF activity and _{c}TF denotes TF surface concentration on cell surface. Let ₐTFₛₜₒₚ denote TF activity that cannot support reverse transmigration. If ₐTFₛₜₒₚ is reached before a foam cell has reached the apical surface of the endothelium, the cell is trapped. Let Δ_{c}TF_{oxLDL}, Δ_{c}TF_{V} denote an increase is TF membrane concentration resulting from stimulation with oxLDL and from microcompetition with a GABP virus, respectively. Let ₐTF_{basal} denote basal TF activity prior to stimulation.

Consider a control cell, denoted “cc,” and a cell harboring a GABP viral genome, denoted “vc.” Microcompetition between the TF promoter and the GABP virus stimulates TF transcription (see the section on TF gene, above). Let t=0 mark the time of monocyte completed differentiation into a macrophage following entry into subendothelial space. For every t>0, microcompetition results in Δ_{c}TF_{V}(t)>0.

In both cells, for every t>0, _{c}TF(t)=_{c}TF_{basal}+Δ_{c}TF(t). However, for viral cell _{c}ΔTF(t)=Δ_{c}TF_{oxLDL}(t)+Δ_{c}TF_{V}(t) (we assume an additive effect for the oxLDL and virus combination). Since Δ_{c}TF_{V}(t)>0 for viral cells, at any time t, TF concentration on surface of a viral cell is greater than TF concentration on surface of control cell.

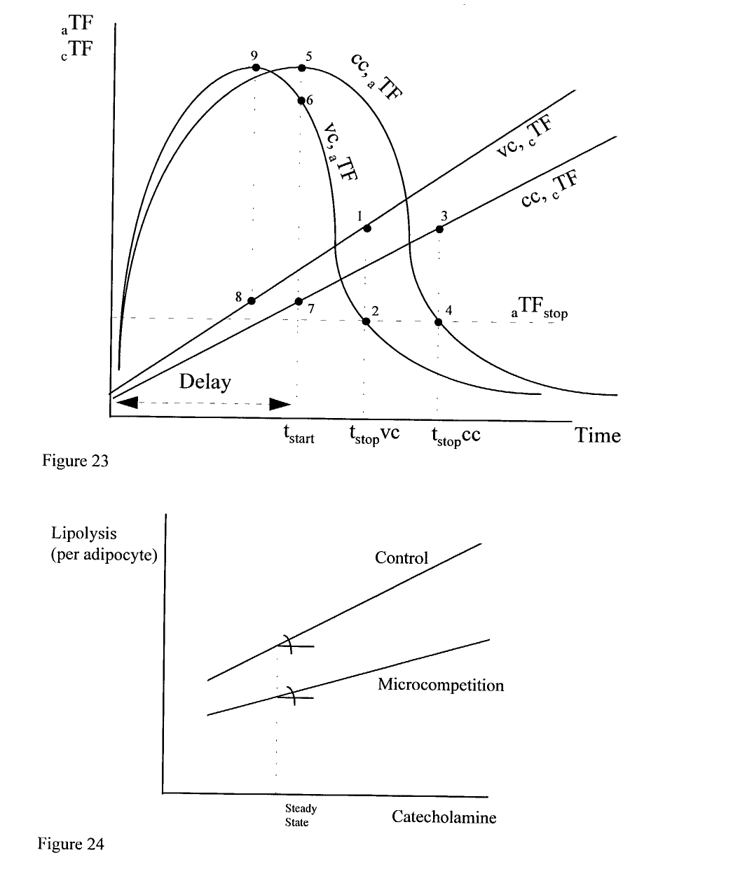

Consider FIG. 23.

“cc, _{c}TF” and “vc, _{c}TF” lines represent the increase in TF surface concentration as a function of time for a control cell and viral harboring cell, respectively. The “cc, ₐTF” and “vc, ₐTF” curves represent the change in TF activity as a function of time for these cells. The vertical distance between “vc, _{c}TF” and “cc, _{c}TF” represents the effect of microcompetition on the surface concentration of TF. The increase in surface TF concentration shifts the “vc, ₐTF” curve to the left. As a rule, in both cells the same TF surface concentration generates the same TF activity. For instance, points 7 and 8 represent the same surface concentration and therefore produce the same activity, represented by points 5 and 9, the points of maximum activity. Points 1 and 3 also represent the same surface concentration. These points produce activity 2 and 4, the activity associated with cells at rest, or “stopped” cells.

For every delay≧0, tₛₜₒₚcc-tₛₜₐᵣₜ>tₛₜₒₚvc-tₛₜₐᵣₜ (see figure). The time during which the viral cell is actually moving towards circulation is shorter compared to control. Assume the probability of reaching the endothelial apical surface increases with movement time. Since the viral cell movement time is shorter, its probability of being trapped is higher.

Another observation relates to cell velocity. Assume the delay is the same for both cells, i.e. cc vc. The shift of the “vc, _{c}TF” curve results in lower TF activity on the viral cell for every t of actual movement (every t>tₛₜₐᵣₜ in the figure). Assume that cell velocity depends on TF activity. Then, at any time, the viral cell is slower than the control cell. The reduced velocity also increases the probability of being trapped.

Microcompetition between a GABP virus and TF increases the probability of being trapped in the subendothelial space. Denote the number of viral N-boxes with V_{Nbox}. Higher V_{NboX} increases the inefficiencies in foam cell backward motility, denoted I in the above clearance model.

Modify equation (2).

$${Rate}_{lesions} = f ( {\%}_{Trapped} ( I ( {V}_{Nbox} ) ) \times {Total}_{FC} \qquad ( 5 )$$

The following derivative represents the effect of V_{Nbox} on Rateₗₑₛᵢₒₙₛ the rate of lesion formation.

$$\frac{\partial R a t {e}_{l e s i o n s}}{\partial {V}_{Nbox}} = \frac{\partial R a t {e}_{l e s i o n s}}{\partial T r a pp e {d}_{F C}} \cdot {Total}_{FC} \cdot \frac{\partial {\%}_{T r app e d}}{\partial I} \cdot \frac{\partial I}{\partial {V}_{Nbox}} \qquad ( 6 )$$

Consider equation (6).

$$\frac{\partial R a t {e}_{l e s i o n s}}{\partial T r a pp e {d}_{F C}} > 0 , T o t a {l}_{F C} > 0 , \frac{\partial {\%}_{T r app e d}}{\partial I} > 0$$

(see above).

$$\frac{\partial {\%}_{T r app e d}}{\partial {V}_{Nbox}} > 0.$$

Therefore,

$$\frac{\partial R a t {e}_{l e s i o n s}}{\partial {V}_{Nbox}} > 0.$$

Microcompetition increases the rate of lesion formation. Moreover, the larger the number of viral N-boxes in the infected cells, the higher the rate of lesion formation.

In addition, CD18 is also a GABP stimulated gene (see above). Therefore, microcompetition between the GABP virus and CD18 gene results in reduced expression of the cellular gene. According to Randolph, et al., (1996, ibid), the role of CD18 is to accelerate the initial kinetics of reverse transmigration (see above). A decrease in CD18 expression might further reduce foam cell velocity, increasing the probability of being trapped in the subendothelial space. Microcompetition therfore has a double impact on reverse transmigration.

(6) Atherosclerosis-Intimal Thickening

A second major class of atherosclerotic lesions is pathological intimal thickening. Intimal thickening consists mainly of smooth muscle cells in a proteoglycan-rich matrix. Pathological intimal thickening should be considered as a class independent of fibrous cap atheroma since the majority of lesion erosion occurs over areas of intimal thickening with minimal or no evidence of a lipid core (Virmani 2000, ibid). Smooth muscle cell (SMC) proliferation, which results in neointima formation and intimal thickening, accounts for a significant rate of restenosis after percutaneous transluminal coronary angioplasty, a widespread treatment for coronary artery disease. The following sections identify the cause of SMC proliferation, neointima formation and intimal thickening in atherosclerosis.

(a) Microcompetition Reduces Rb Transcription in SMC

SMCs are permissive to HCMV (Zhou 1996⁴¹⁷) and HSV (Benditt 1983⁴¹⁸). Rb is a GABP stimulated gene. Microcompetition with viral DNA decreases Rb transcription in SMCs (see the section on cancer).

(b) Reduced Rb Expression in Atherosclerotic Plaque

Rb mRNA is reduced in atherosclerotic plaque. Consider the following study.

Rabbits were fed a high cholesterol diet for six months. The results showed that the atherosclerotic plaques, covering 91% of the intimal aortic surface of aorta thoracalis, contained less Rb mRNA (P<0.05) compared to normal aortic arteries (Wang 1996⁴¹⁹). Based on this result, Wang, et al., suggested that “the abnormal expression of . . . Rb antioncogene may play an important role in arterial SMC proliferation and pathogenesis of atherosclerosis.”

(c) Increased pRb Expression Reduces Neointima Formation

Rb is important in SMC arrest and differentiation. Increased Rb transcription (Claudio 1999⁴²⁰, Schwartz 1999⁴²¹, Smith 1997⁴²²), or reduced pRb phosphorylation (Gallo 1999⁴²³), decreased SMC proliferation and neointima formation. Since microcompetition reduces Rb transcription, an infection with a GABP virus results in SMC proliferation, neointima formation and pathological intimal thickening.

(7) Thrombosis

Plaque rupture may lead to in situ formation of a thrombus. The rupture exposes the TF excessively expressed on surface of foam cells. The exposed TF triggers the coagulation event.

(8) Viruses in Atherosclerosis

The idea of infection as a risk factor for atherosclerosis and related cardiovascular diseases is more than 100 years old. However, it was not until the 1970s that experimental data was published supporting the role of viruses in atherosclerosis. The mounting evidence linking infectious agents and atherosclerosis prompted the scientific community to organize the International Symposium of Infection and Atherosclerosis, held in Annecy, France, Dec. 6-9, 1998. The main objective of the symposium was to evaluate the role of infection in the induction/promotion of atherosclerosis on the basis of evidence from recent data on pathogenesis, epidemiologic and experimental studies, to define prevention strategies and promote further research. Consider the following studies presented at the symposium. The studies were published in a special issue of the American Heart Journal (see American Heart Journal, November 1999).

Chiu presented a study that found positive immunostainings for C pneumoniae (63.6%), cytomegalovirus (CMV) (42%), herpes simplex virus-1 (HSV-1) (9%), P gingivalis (42%), and S sanguis (12%) in carotid plaques. The study found 1 to 4 organisms in the same specimen (30%, 24%, 21%, and 6%, respectively) and the microorganisms were immunolocalized mostly in macrophages (Chiu 1999⁴²⁴).

In a critical review of the epidemiologic evidence, Nieto suggested that “most epidemiologic studies to date (Nieto 1999⁴²⁵, Table I and II) have used serum antibodies as surrogate indicators of chromic viral infection. However, there is evidence suggesting that serum antibodies may not be a valid or reliable indicator of chromic or latent infections by certain viruses. In a athological study of patients undergoing vascular surgery for atherosclerosis serology, for example, for the presence of serum cytomegalovirus antibodies was not related to the presence of cytomegalovirus DNA in atheroma specimens.” However, according to Nieto, four studies, Adam, et al., (1987⁴²⁶), Li, et al., (1996⁴²⁷), Liuzzo, et al., (1997⁴²⁸) and Blum, et al., (1998⁴²⁹) showed strong positive associations between CMV infection and clinical atherosclerosis. A strong association was also found in a 1974 survey of the participants in the Atherosclerosis Risk in Communities (ARIC) study between levels of cytomegalovirus antibodies and the presence of subclinical atherosclerosis, namely carotid intimal-medial thickness measured by B-mode ultrasound (Nieto 1999, ibid).

Nieto also reported results of a prospective study of clinical incident coronary heart disease (CHD). The study was a nested case-control study from the Cardiovascular Health Study (CHS) conducted in an elderly cohort. Preliminary results from this study found no association between cytomegalovirus antibodies at baseline and incident CHD over a 5 year period. However, HSV-1 was strongly associated with incident CHD, particularly among smokers (odds ratio [OR] 4.2). It should be noted that a more recent prospective study of CMV, HSV-1 in CHD found that participants in the Atherosclerosis Risk in Communities Study (ARIC) study with highest CMV antibody levels at base line (approximately in the upper 20%) showed increased relative risk (RR, 1.76, 95% confidence interval, 1.00-3.11) of CHD incidence over a 5 year period, adjusted for age, sex and race. After adjustment for additional covariates of hypertension, diabetes, years of education, cigarette smoking, low-density lipoprotein and high-density lipoprotein cholesterol levels, and fibrinogen level, the RR increased slightly. (The study found no association between CHD and the highest HSV-1 antibody levels (adjusted RR, 0.77; 95% confidence interval, 0.36-1.62) (Sorlie 2000⁴³⁰)).

Nieto (1999, ibid) also mentioned some recent studies, which documented increased risk of restenosis after angioplasty in patients with serologic evidence of cytomegalovirus infection. For instance, Nieto reported a study by Zhou and colleagues, which included 75 consecutive patients undergoing directional coronary atherectomy for symptomatic coronary artery disease. Six months after atherectomy, the cytomegalovirus-seropositive patients showed significantly greater reduction in luminal diameter and significantly higher rate of restenosis compared to controls (43% vs 8% OR 8.7). These results were independent of known cardiovascular disease (CVD) risk factors.

Finally, Nieto mentioned that cytomegalovirus infection has been associated with another form of atherosclerotic disease: accelerated atherosclerosis in the coronaries after heart transplantation. In the first study showing this association, cytomegalovirus serology after transplantation seemed to be one of the most significant predictors of graft atherosclerosis and survival in general. This difference was independent of serologic status before transplantation and the presence of symptomatic infection. Similar results have been replicated in subsequent studies.

Based on these studies Nieto concludes that “despite its limitations, the epidemiologic evidence reviewed above is consistent with a broad range of experimental and laboratory evidence linking viral (and other) infections and atherosclerosis disease.”

In a review of animal studies, Fabricant, et al., (1999⁴³¹) described their experiments with Marek's disease herpesvirus (MDV). The initial experiment used 4 groups of specific pathogen-free (SPF) white leghorn chickens, β-line cockerels of the same hatch, genetically selected for susceptibility to MDV infection. Groups 1 and 2 were inoculated intratracheally at 2 days of age with 100 plaque-forming units of clone-purified, cell free, CU-2 strain of low-virulence MDV. Groups 3 and 4 were controls. For the first 15 weeks, all birds in the 4 groups were fed the same commercial low cholesterol diet (LCD). Beginning with the 16th and ending with the 30th week, MDV-infected group 2 and uninfected group 4 were placed on a high cholesterol diet (HCD). The other two groups remained on LCD. Atherosclerotic lesions visible at gross inspection were only observed in MDV-infected birds of groups 1 (LCD) and 2 (HCD). These arterial lesions were found in coronary arteries, aortas, and major arterial branches. In some instances, the marked atherosclerotic changes involved entire segments of the major arteries practically occluding the arterial lumen. Other arterial lesions visible at gross inspection were observed as discrete plaques of 1 to 2 mm. These arterial lesions were not found in any of the uninfected birds of group 3 (LCD) or the uninfected hypercholestrolemic birds of group 4. Many proliferative arterial lesions with intimal and medial foam cells, cholesterol clefts, and extracellular lipid and calcium deposits had marked resemblance to chronic human atherosclerotic lesions. Moreover, immunization against MDV prevented the MDV-induced atherosclerotic lesions.

The main conclusion of the symposium was that “although studies are accumulating that indicate a possible relation between infection and atherosclerosis, none of them has yet provided definite evidence of a causal relationship . . . . Moreover, the demonstration of a causative role of infectious agents in atherosclerosis would have an enormous impact on public health” (Dodet 1999⁴³²) (A similar view is expressed in a review published recently, see Fong 2000⁴³³).

What is “definitive evidence?” What evidence will convince Dodet, and others, that viruses are not merely associated with atherosclerosis but actually cause the disease?

The research on viruses in cancer provides an answer. According to zur Hausen (1999⁴³⁴) “The mere presence of viral DNA within a human tumor represents a hint but clearly not proof for an aetiological relationship. The same accounts for seroepidemiological studies revealing elevated antibody titres against the respective infection.” What constitutes a proof is evidence that meets the following four criteria, especially the fourth one. According to zur Hausen “the fourth point could be taken as the most stringent criterion to pinpoint a causal role of an infection.”

TABLE 1
zur Hausen's criteria for defining
a causal role for an infection in cancer

| | |
|---|---|
| 1. | Epidemiological plausibility and evidence that a virus |
| | infection represents a risk factor for the development |
| | of a specific tumor. |
| 2. | Regular presence and persistence of the nucleic acid of |
| | the respective agent in cells of the specific tumor. |
| 3. | Stimulation of cell proliferation upon tranfection of the |
| | respective genome or parts therefrom in corresponding |
| | tissue culture cells. |
| 4. | Demonstration that the induction of proliferation and |
| | the malignant phenotype of specific tumor cells depends |
| | on functions exerted by the persisting nucleic acid of |
| | the respective agent. |

The fourth point requires an understanding of the “mechanisms of virus mediated cell transformation.” Crawford (1986⁴³⁵) and Butel (2000, ibid) also emphasize the significance of understanding the mechanism in attributing a causal role to infection. According to Crawford: “one alternative approach to understanding the role of the papillomaviruses in cervical carcinoma is to identify the mechanisms by which this group of viruses may induce the malignant transformation of normal cells.” According to Butel: “molecular studies detected viral markers in tumors, but the mechanism of HBV involvement in liver carcinogenesis remains the subject of investigation today.” When the other kind of evidence is in place, understanding the mechanism turns a mere association into a causal relation.

The discovery of microcompetition and its effect on macrophage propulsion and SMC replication provides the mechanism that produces atherosclerosis. This discovery supplies the missing “definitive evidence” for a causal relationship between viruses and atherosclerosis.

(c) Metastasis

(1) Increased TF Expression Promotes Metastasis

The expression of TF is increased in various metastatic tumors such as non-small-cell lung cancers (Sawada 1999⁴³⁶), colorectal cancer (Shigemori 1998⁴³⁷), melanoma (Meuller 1992⁴³⁸), prostate cancer (Adamson 1993⁴³⁹), colorectal carcinoma cell lines and metastatic sublines to the liver (Kataoka 1997⁴⁴⁰), breast cancer (Sturm 1992⁴⁴¹), and in a variety of cancer cell lines (Hu 1994⁴⁴²). Moreover, TF expression directly correlates with tumor aggressiveness (see above studies and following reviews, Ruf 2000⁴⁴³, Schwartz 1998⁴⁴⁴).

In an intervention study which generated two matched sets of cloned human melanoma lines, one expressing a high level and the other a low level of normal human TF molecule, by retroviral-mediated transfections of a nonmetastatic parental line. The tumor cells were injected into the tail vein of severe combined immunodeficiency (SCID) mice. The results showed that metastatic tumors in 86% of the mice injected with the high-TF lines and in 5% of the mice injected with the low-TF lines (Bromberg 1995⁴⁴⁵). Based on these results, Bromberg, et al., concluded that “high TF level promotes metastasis of human melanoma in the SCID mouse model.”

(2) Microcompetition Increases TF Transcription, and Therefore, Metastasis

TF is a GABP suppressed gene. Microcompetition increases TF transcription (see above). Therefore, an infection with a GABP virus promotes metastasis.

(d) Osteoarthritis

(1) Mutation Studies

(a) Collagen Type I α₂ Chain (COL1A2)

(i) COL1A2 is a Microcompetition-Repressed Gene

See above (the study with viral plasmid).

Moreover, the COL1A2 is ERK responsive. ERK stimulates COL1A2 transcription. One study examined the influence of hypergravity on collagen synthesis in human osteoblast-like cells (hOB), as well as the involvement of the MAP kinase signaling cascade. They found that hypergravity led to significantly increased phosphorylation of ERK 1/2. When the MAPK kinase pathway was inhibited by PD98059, hypergravity-induced stimulation of both collagen synthesis as well as COL1A2 mRNA expression decreased by about 50% (Gebken 1999⁴⁴⁶).

(b) COL1A2 Deficiency

(i) COL1A2 Causes EDS

A latent infection by a GABP virus results in microcompetition between viral DNA and the COL1A2 gene, which decreases the expression of the cellular gene (see above). A heterozygous mutation of the COL1A2 gene causes the Ehlers-Danlos syndrome type-VII. EDS patients suffer from COL1A2 protein deficiency. Therefore, research on EDS type-VII can be used to gain insights on the effects of a GABP viral infection on animal and human health.

(a) EDS is Associated with Hypermobility of Certain Joints

The COL1A2 deficieny in EDS type-VII causes hypermobility ofjoints (Byers 1997⁴⁴⁷, Giunta 1999⁴⁴⁸). A hypermobilejoint is defined as ajoint whose range of movement exceeds the norm for that individual, taking into consideration age, sex, and ethnic background. The primary cause of hypermobility is ligamentous laxity, which is determined by each person's fibrous protein genes (Grahame 1999⁴⁴⁹).

A high concentration of collagen type I, 55-65% of dry weight, is found in the matrix components of interarticular fibrocartilages (menisci) tissues. Meniscus tissues are found in the temporomandibular, temoclavicular, acromiocalvicular, wrist and knee joints. High concentration of collagen type I is also found in connecting fibrocartilages, such as vertebrae discs. As a result of COL1A2 deficiency, these joints show a higher degree of hypermobility compared to other joints. We call the temporomandibular, ternoclavicular, acromiocalvicular, wrist, knee and lumber joints the “Vulnerable Joints.”

(b) Hypermobility in Obesity

A latent infection by a GABP virus results in microcompetition between viral DNA and the COL1A2 gene which decreases the expression of COL1A2. A COL1A2 deficiency causes hypermobility in vulnerable joints, specifically, in the lumbar joints. A infection also results in decreased expression of the hMT-II_{A} gene and obesity (see above). Therefore, obese people should show hypermobility in their lumbar joints.

A modified Schober test was used to examine lumbar mobility. To perform the test, the subjects were first asked to stand erect. While erect, three marks were placed on the subject's skin overlaying the lumbosacral spine. The first mark was placed at the lumbosacral junction, the second mark was placed 5 cm below the first, and the third mark was placed 10 cm above the junction. The subject was then asked to bend forward as far as possible, as though to touch the toes. The new distance between the second and third mark was measured. Lumbar mobility is defined as the difference between this measurement and the initial distance of 15 cm. The study group included 2,350 men and 670 women between the ages of 21 and 67 years.

Obesity (defined as weight/height) markedly affected the flexibility measurements. For every increase in obesity by one standard deviation, an increase of 0.4 cm was measured in the modified Schober measurement. The results showed that younger subjects are more mobile in their lumbar joints. Female subjects in their 20's showed an increase of 0.42 cm in the modified Schober measurement compared to female in their 60's. Man showed a 1.04 cm increase over the same age difference. The increased flexibility demonstrated by the most obese subjects (top 16%, or 1 SD of weight/height subjects) is equal to the increase in flexibility associated with 40 year age difference in female (0.4 cm compared to 0.42 cm), and is almost half the increase associated with that age difference in men (0.4 cm compared to 1.04 cm) (Batti'e 1987⁴⁵⁰).

(c) Hypermobility Causes Osteoarthritis

A study with EDS patients found that 16 out of 22 over the age of 40 have osteoarthritis of one or more joints (referenced in Grahame 1989⁴⁵¹). In the general population, evidence is more circumstantial. However, the Leeds groups produced evidence of a likely association between joint laxity and osteoarthritis (OA). The study compared 50 women with symptomatic OA to age matched controls. The study found a direct correlation between developing OA and the degree of hypermobility (Scoott 1979⁴⁵²).

The association between hypermobility and osteoarthritis was studied in specific joints. Sharma, et al., (1999⁴⁵³) report that laxity is greater in the uninvolved knees of OA patients compared to knees of older controls. The authors concluded that at least some of the increased laxity of OA may predate the disease. Jonsson, et al., (1996⁴⁵⁴) compared 50 female patients with clinical thumb base (first carpometacarpal joint) OA to age matched controls. The results showed that hypermobility features were much more prevalent in the 50 patients compared to controls. The authors also report another study with 100 patients (including both males and females) that found a direct correlation between hypermobility and clinical severity of thumb base OA. They concluded that a causal relationship existes between articular hypermobility and thumb base OA.

(d) Osteoarthritis in Obesity

Microcompetition causes hypermobility, which causes osteoarthritis in vulnerable joints. Microcompetition also causes obesity. Therefore, obese people should show osteoarthritis in vulnerable joints.

A study compared the OA disease traits in different joints of female twins aged 48-70. The results showed that, in twins, an increase in the body weight increased the likelihood of developing osteoarthritis in the knee in both the tibiofemoral joint (TFJ) and patellofemoral joint (PFJ) and in the hand in the first carpometacarpal joint (CMC I). Specifically, after adjustment for other potential risk factors, for every 1 kg increase in body weight a twin had a 14% increased risk of developing TFJ osteophytes, a 32% increased risk of developing PFJ osteophytes, and a 10% increased risk of developing CMC osteophytes compared to their co-twin. Moreover, the weight difference was also observed in asymptomatic woman, which indicates that weight gain predates OA and, therefore, is not a result of OA (Cicuttini 1996⁴⁵⁵).

Note that this twin study demostrates an association betweeen obesity and OA independent of genetic factors, and is, therefore, inconsistent with the genetic mutation explantion of obesity (see above).

A longitudinal study began in 1962 with baseline examinations of clinical, biochemical, and radiologic characteristics. In 1985 follow-up examination characterized osteoarthritis in 1,276 participants, 588 males and 688 females, ages 50-74. Baseline obesity was measured by an index or relative weight. The results showed that the likelihood of developing osteoarthritis of the hand over the 23-year period increased with an increase in the index measuring baseline relative weight. Higher baseline relative weight was also associated with greater subsequent severity of the disease. Moreover, during the 23-year period, most subjects gained weight. However, after adjustment for baseline weight, the increase in body weight was not associated with either the likelihood of developing osteoarthritis of the hand or the severity of the disease, which indicates that OA is not a result of weight gain (Carman 1994⁴⁵⁶).

In obesity some joints seem to be susceptible to osteoarthritis while other are protected. The knees and the thumb base, for intance, are often damaged while the hips are disease free. Since both are weight-bearing joints, the difference in susceptibility to osteoarthritis indicates a cause other than mechanical wear-and-tear. The pattern of OA in obesity also does not correspond to a general metabolic cause for the disease. A metabolically induced deterioration of cartilage should result in small differences in the severity of OA between joints, unlike the differences observed in joints of obese people. van Sasse, et al., call the pattern of OA in obesity “strange,” and claims that “whatever the final explanation for the etiology of OA, we believe that it will have to take into account the strange pattern of the association between OA and obesity” (van Saase 1988⁴⁵⁷).

These studies suggest three insights. First, obesity is associated with osteoarthritis in only specific joints—van Saase's “strange” list of susceptible joints. Second, obesity and osteoarthritis do not a result from of each other. Third, the association between obesity and osteoarthritis is independent of genetic factors. Obesity and OA resulting from microcompetition between viral and cellular DNA is consistent with all three insights. First, van Saase's “strange” list of susceptible joints coincides with the list of vulnerable joints. Second, both obesity and OA result from microcompetition and not from each other. Last, microcompetition results from a viral infection and not from a genetic mutation.

(c) Collagen Type I α₂ Chain (COLIA2), Obesity and Obstructive Sleep Apnea (OSA)

Obesity is associated with hypermobility of vulnerable joints. The temporomandibular joint belongs to the list of vulnerable joints. Therefore, in obesity the temporomandibular joint is hypermobile.

The mandible and tongue protrusion of obese patients was compared to controls. The subject was asked to protrude the mandible or tongue as far forward as possible (MAX), and 50% was measured as the midpoint between maximum protrusion and the position were the tongue tip rests between the incisors (50%). The difference between resting position R and MAX and between R and 50% is denoted R-MAX and R-50%, respectively. The results showed that obese subjects differed from controls in the degree of change in cross-sectional area (CSA) in the oropharynx. The 50% mandibular protrusion (R-50%) and the maximum tongue protrusion (R-MAX) produced greater relative increases in oropharyngeal cross-sectional area in obese subjects compared to controls (Ferguson 1997⁴⁵⁸). Increased oropharyngeal cross-sectional area indicates an increased capacity for mandibular protrusion. Such increased capacity indicates hypermobility of the temporomandibular joint.

During sleep, the tonic activity of the masseter decreases. In a supine position the mandible drops and the mouth opens. A hypermobile temporomandibular joint lets the mandibular drop further and the mouth open wider than a normal joint.

A study compared the time spent with mandibular opening in OSA patients and healthy controls. In controls, 88.9% of total sleep time was spent with narrow mandibular opening (less than 5 mm). In contrast, in OSA patients, 69.3% of the total sleep time was spent with wide mandibular opening (more than 5 mm). Moreover, in healthy adults, there was no difference in mandibular posture between the supine and lateral recumbent positions, while in OSA patients, sleep stage affects the mandibular opening during sleep in the supine position only (Miyamoto 1999⁴⁵⁹).

The abnormal low position of the hypermobile mandibular causes the upper airway disturbances during sleep. Therefore, hypermobility of the temporomandibular joint causes OSA.

Without reference to hypermobility of the temporomandibular joint, Miyamoto, et al., (1999) proposes a similar description of the events leading to apnoeic episodes.

Microcompetition causes obesity. Microcompetition also causes hypermobility of the temporomandibular joint, which causes OSA. Therefore, obesity is associated with OSA (note that the OSA patients in Ferguson, et al., (1997, ibid) and Miyamoto, et al., (1999) studies above are obese).

(e) Obesity

(1) Background

(a) The Obesity Epidemic

“The prevalence of obesity (defined as body mass index≧30 kg/m²) increased from 12.0% in 1991 to 17.9% in 1998. A steady increase was observed in all states; in both sexes; across age groups, races, education levels; and occurred regardless of smoking status” (Mokdad 1999⁴⁶⁰).

(b) Three Proposed Causes for the Epidemic

As proposed throughout the scientific community, the three “classical” causes of the obesity epidemic are increased energy intake, reduced energy expenditure, and genetic mutation.

(i) Increased Energy Intake (“too much Food”)

Many large-scale studies refute the idea that increased energy intake is the cause of obesity. The USDA Natlonwide Food Consumption Survey 1977-1988 collected data from over 10,000 individuals. The analysis found that the average fat intake in the United States decreased from 41% to 37% of calorie intake between 1977 and 1988 and the average total energy intake decreased, by 3% in women and by 6% in men. “The reductions in average fat and energy intake were associated with a progressive increase in the prevalence of obesity in the US adult population” (Weinsier 1998⁴⁶¹).

An even larger study reported similar results based on pooled data from NHANES II and III, USDA Natlonwide Food Consumption Survey, Behavioral Risk Factor Survey System, and Calorie Control Council Report (Heini 1997⁴⁶²). “In the adult US population the prevalence of overweight rose from 25.4% from 1976 to 1980 to 33.3% from 1988 to 1991, a 31% increase. During the same period, average fat intake, adjusted for total calories, dropped from 41.0% to 36.6%, an 11% decrease. Average total daily calorie intake also tended to decrease, from 1,854 kcal to 1,785 kcal (−4%). Men and women had similar trends. Concurrently, there was a dramatic rise in the percentage of the US population consuming low-calorie products, from 19% of the population in 1978 to 76% in 1991” (Ibid). The authors conclude that “reduced fat and calorie intake and frequent use of low-calorie food products have been associated with a paradoxical increase in the prevalence of obesity” (Ibid). Similar surveys conducted in Great Britain corroborate these studies.

(ii) Reduced Energy Expenditure (“too Liftle Exercise”)

Many have turned their attention to reduced physical activity as an alternative explanation for the obesity epidemic. “The only available explanation for the paradoxical increase in body weight with a decrease in fat and energy intake is that physical activity declined” (Ibid). The data disprove this explanation as well.

In recent years several population surveys have shown unchanging levels of physical activity among Americans. For example, in the Behavioral Risk Factor Survey which included 30,000 to 80,000 individuals annually, the prevalence of obesity increased from 12% to 17.9% between 1991 and 1998 but physical inactivity did not change substantially (Ibid).

(iii) Genetic Mutation

“The fact that the increased rates of obesity have been observed within the last two decades has been viewed as evidence that genetic factors cannot be held responsible. Indeed, systematic changes of the population-based frequencies of specific alleles predisposing to obesity cannot possibly have occurred within this short time span.” (Hebebrand 2000⁴⁶³) A significant change in the human gene pool requires many generations. A genetic mutation explanation for the increase in obesity implies that the human gene pool has changed over a single generation. “Although research advances have highlighted the importance of molecular genetic factors in determining individual susceptibility to obesity, the landmark discoveries of leptin, uncoupling proteins and neuropeptides involved in body weight regulation, cannot explain the obesity epidemic” (Hill 1998⁴⁶⁴). “Genes related to obesity are clearly not responsible for the epidemic of obesity because the gene pool in the United States did not change significantly between 1980 and 1994” (Koplan 1999⁴⁶⁵).

(2) Knockout Studies

(a) Human Metallothionein-II_{A} (hMT-II_{A})

(i) hMT-II_{A} is a Microcompetition-Suppressed Gene

A latent infection by a GABP virus results in microcompetition between the viral DNA and the hMT-II_{A} gene which decreases the expression of the cellular gene (see above). A disruption of the metallothionein gene in transgenic mice also reduces the expression of the cellular gene. Therefore, research with MT-null mice can produce insights on the effects of a GABP viral infection on animal and human health.

(ii) MT-I and MT-II Null Mice are Obese

Mice with disrupted MT-I and MT-II genes are apparently phenotypically normal. The disruption shows no adverse effect on the ability to reproduce and rear offspring. However, after weaning, MT-null mice consume more food and gain more weight at a more rapid rate than control mice. The majority of the adult male mice in the MT-null colony show moderate obesity (Beattie 1998⁴⁶⁶).

(b) Integrin (β₂ Leukocyte, CD18)

Notations and Terminology:

β₂=CD18

α_{L}=CD11a (L for Leukocytes) expressed in all leukocytes

α_{M}=CD11b (M for Monocytes/Macrophage) expressed in monocytes/macrophages, granulocytes, natural killer cells, a sub population of T cells

LFA-1=Lymphocyte-Function-associated Antigens 1

MAC-1=Macrophage 1

CR3=Complement Receptor type 3

α_{L}β₂=CD11a/CD18=LFA-1 (LFA-1 binds ICAM-1 and ICAM-2)

α_{M}β₂=CD11b/CD18=MAC-1=CR3=Mo-1 (MAC-1 binds ICAM-1, C3b, fibrinogen and factor X)

(i) CD18 is a Microcompetition-Suppressed Gene

CD18 is a leukocyte-specific adhesion molecule. GABP binds three N-boxes in the CD18 promoter and transactivates the gene (Rosmarin 1995, ibid, Rosmarin 1998, ibid). Since CD18 is a GABP stimulated gene, latent infection by a GABP virus results in microcompetition between the viral DNA and the CD18 promoter thereby decreasing the expression of CD18 (Le Naour 1997, ibid, Tanaka 1995, ibid, Patarroyo 1988, ibid, see above). Moreover, the higher the concentration of viral DNA, the greater the decrease in CD18 expression.

(ii) ICAM-1 or MAC-1 Null Mice are Obese

CD18 participates in forming the CD11a/CD18 molecule that binds ICAM-1. ICAM-1 null mice (ICAM-1−/−) gain more weight than control mice after 16 weeks of age, and eventually became obese despite no obvious increase in food intake. Under a high fat diet, ICAM-1−/− mice show an increased susceptibility to obesity. CD18 also participates in forming the CD11b/CD18 molecule that binds MAC-1. MAC-1 null mice (MAC-1−/−) are also susceptible to diet-induced obesity and exhibited a strong similarity in weight gain with sex-matched ICAM-1−/− mice (Dong 1997⁴⁶⁷).

(3) Pathogenesis

(a) Hormone Sensitive Lipase (HSL) Gene

(i) HSL is a Microcompetition-Suppressed Gene

See above.

(ii) Reduced HSL mRNA in Obesity

HSL mRNA, protein expression, and enzyme activity were measured in abdominal subcutaneous adipocytes from 34 obese drug-free and otherwise healthy males and females and 14 non-obese control subjects. The results showed reduced HSL mRNA, protein expression and enzyme activity (Large 1999⁴⁶⁸, Table 3). The findings were age and gender independent. Based on these results Large, et al., conclude that “a decreased synthesis of the HSL protein at the transcriptional level is a likely factor behind the findings of decreased HSL expression in adipocytes from obese subjects . . . . Decreased HSL expression may at least in part explain the well-documented resistance to the lipolytic effect of catecholamines in obesity.”

In line with these results, a subsequent study by the same laboratory showed a 73% reduction in HSL protein levels in obesity (Elizalde 2000⁴⁶⁹, FIG. 4C and Table 1).

(iii) Catecholamines Resistance in Obesity

(a) HSL regulation

Catecholamines bind β₁, β₂- and β₃-adrenergic receptors (β₁AR, β₂AR and β₃AR, respectively) and α₂ adrenergic receptors (α₂AR).

(i) Transcription

Activation of β₂AR (Maudsley 2000⁴⁷⁰, Pierce 2000⁴⁷¹, Elorza 2000⁴⁷², Luttrell 1999⁴⁷³, Daaka 1998⁴⁷⁴) or β₃AR (Cao 2000⁴⁷⁵, Gerhardt 1999⁴⁷⁶, Soeder 1999⁴⁷⁷) activates ERK which phosphorylates GABP which in turn binds p300, resulting in increased HSL transcription.

(ii) Post-Translation

Activation of β₁AR, β₂AR, β₃AR activates a cAMP dependent protein kinase A. The protein kinase phosphorylates HSL, resulting in increased hydrolytic activity against triacylglycersol and cholesteryl ester substrates. Insulin deactivates HSL via protein phosphatases or inhibition of protein kinase.

(b) Reduced Response to Stimulation

(i) Hypothesis

Microcompetition reduces HSL expression. Since HSL is rate limiting in triacylglycerol and diacylglycerol hydrolysis, microcompetition reduces steady state lipolysis. Moreover, as ERK agents, β₂AR and β₃AR agonists, specifically catecholamines, stimulate HSL transcription. Microcompetition also lessens the increase in HSL transcription, resulting in impaired stimulated lipolysis. Consider FIG. 24.

At steady state, microcompetition reduces lipolysis per adipocyte. Microcompetition also reduces the slope of the lipolysis line. That is, with increased stimulation, the relative lipolysis deficiency (the vertical difference between the two lines) increases.

A number of in vivo and in vitro studies demonstrated the reduced ability of catecholamines to stimulate lipid mobilization from subcutaneous adipose tissue.

(ii) In vitro Studies

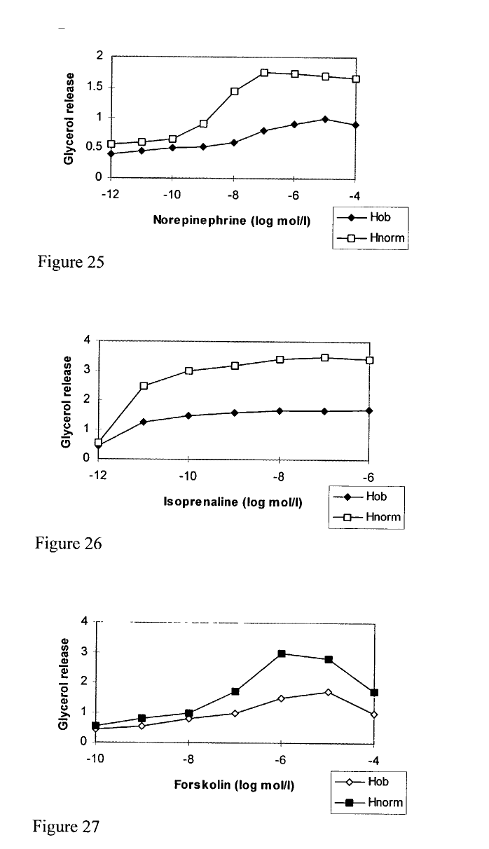

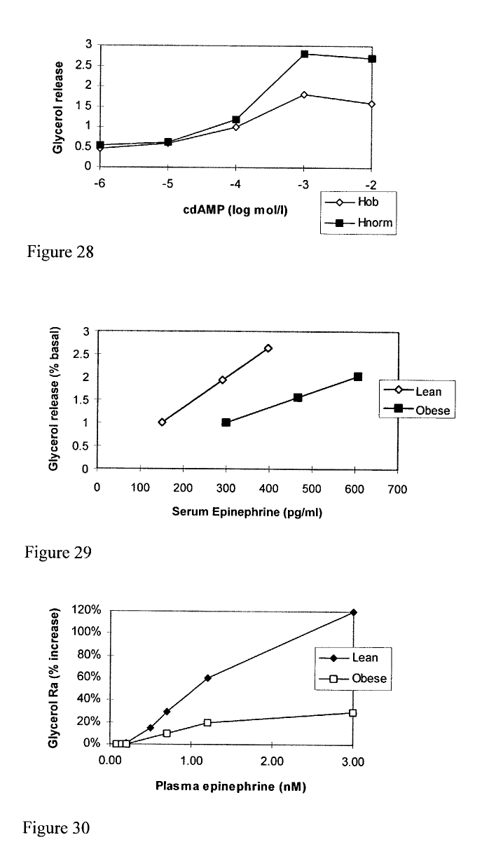

Hellstrom, et al., (1996⁴⁷⁸) treated abdominal subcutaneous adipocytes from 13 non-obese subjects with at least one first-degree relative with body mass index of 27 kg/m² or more (Hob) and 14 controls (Hnorm) with norepinephrine, a major endogenous lipolytic agent, isoprenaline, a non-selective beta-adrenoceptor agonist, forskolin, a direct activator of adenylyl cyclase, and dibutyryl cyclic AMP, an activator of protein kinase and thereby HSL. FIGS. 25, 26, 27 and 28 represent the effect of these treatments on the glycerol release (pmol·cell·2 h⁻¹) from adipocytes.

The average rate of lipolysis induced by all four treatments was reduced by about 50% (p from 0.001 to 0.01) in subjects with a family trait of obesity compared to controls. Isoprenaline (Shimizu 1997⁴⁷⁹), dibutyryl cAMP (Shimizu 1997) and forskolin (Yarwood 1996⁴⁸⁰) activated ERK in adipocytes. Isoprenaline also activated ERK in CHO/K1 cells expressing the human β₃AR (Gerhardt 1999⁴⁸¹). As ERK agents the agonists phosphorylate GABP. Microcompetition in obese adipocytes reduces the maximum number of GABP molecules available for HSL promoter binding, hence, the observed resistance for these agonists stimulation. Moreover, as expected, an increase in the agonist concentration increased the relative lipolysis deficiency.

Hellstrom, et al, (1996) also measured the HSL maximum activity and HSL mRNA at steady state. The maximum activity was reduced 50% in Hob (p<0.05). mRNA (amol HSL/μg total nucleic acids) was reduced by 20% (p>0.05, not significant). The study did not measure HSL mRNA after stimulation.

The following studies use the concept of maximum adipocyte lipolysis capacity in response to stimulation by various agonists by comparing glycerol release in adipocytes from obese male and female to controls. In all studies the adipocyte incubation in the presence of the agonist lasted 2 h.

Large, et al., (1999, ibid) treated abdominal subcutaneous adipocytes from 34 obese drug-free and otherwise healthy males or females and 14 non-obese controls, with isoprenaline, a non-selective β-adrenergic receptor agonist, or dibutyryl cAMP, a phosphodiesterase resistant cAMP analogue. The results showed reduced maximum values for isoprenaline- and dibutyryl cAMP induced glycerol release by 40-50% in the obese group, when expressed per g lipid.

Hellstrom, et al., (2000⁴⁸²) treated abdominal subcutaneous adipocytes from 60 obese and 67 non obese subjects, age 19-60 y, with isoprenaline, dibutyryl cyclic AMP, and forskolin, an activator of adenylyl cyclase. The results showed reduced maximum values for isoprenaline-, dibutyryl cAMP-, and forskolin induced glycerol release by 50% in the obese group. Moreover, 42 of the 67 lean subjects had at least one obese member among first-degree relatives, but not all family members, and not both parents. The non-obese subject with the family trait for obesity showed a similar reduction in maximum glycerol release compared to lean subjects without the family trait.

(iii) In vivo Studies

Consider Bougneres 1997⁴⁸³. To study the effect of epinephrine on lipolysis in obesity, epinephrine was infused stepwise at fixed doses of 0.75 and then 1.50 μg/min to 9 obese children (160+/−5% ideal body weight) aged 12.1+/−0.1 yr during the dynamic phase of fat deposition, and in 6 age-matched non-obese children. As an in vivo lipolysis index, the study used glycerol flux. In the basal state, obese children had a 30% lower rate of glycerol release per unit fat mass than lean children. FIG. 29 represents the measured relationship between epinephrine infusion and glycerol release.

Consider Horowitz (2000⁴⁸⁴). Lipolytic sensitivity to epinephrine was measured in 8 lean [body mass index (BMI): 21±1 kg/m²] and 10 upper body obese (UBO) women (BMI: 38±1 kg/m²; waist circumference>100 cm). All subjects underwent a four-stage epinephrine infusion (0.00125, 0.005, 0.0125, and 0.025 microgram·kg fat-free mass⁻·min⁻¹) plus pancreatic hormonal clamp. Glycerol rates of appearance (Rₐ) in plasma were determined by stable isotope tracer methodology. FIG. 30 represents the measured percent change in glycerol release as a function of plasma epinephrine concentration.

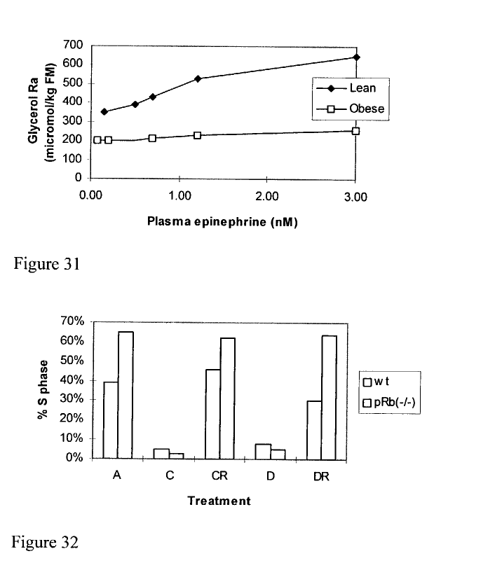

FIG. 31 represents the same results in terms of total glycerol release per fat mass (FM).

Both the Bougneres (1997) and Horowitz (2000) results are consistent with microcompetition as the underlying cause of catecholamine resistance in obesity.

(iv) Adipocyte Hypertrophy in Obesity

HSL is a GABP regulated gene. Microcompetition reduces HSL expression, which results in adipocyte hypertrophy. Consider the following study.

HSL knockout mice were generated by homologous recombination in embryonic stem cells. Cholesterol ester hydrolase (NCEH) activities were completely absent from both brown adipose tissue (BAT) and white adipose tissue (WAT) in mice homozygous for the mutant HSL allele (HSL−/−). The cytoplasmic area of BAT adipocytes was increased 5-fold in HSL−/− mice (Osuga 2000⁴⁸⁵, FIG. 3a). The median cytoplasmic area in WAT was enlarged 2-fold (Ibid, FIG. 3b). The HSL knockout mice showed adipocyte hypertrophy.

Obesity is characterized by adipocyte hypertrophy. Osuga (2000) results are consistent with microcompetition as the underlying cause of adipocyte hypertrophy in obesity.

It is interesting that body weight of the HSL−/− mice was not different, at least until 24 weeks of age, from wild type. The reason was probably lack of adipocyte hyperplasia in HSL−/− mice. Consider the following section.

(b) Retinoblastoma Susceptible Gene (Rb)

(i) Rb is a Microcompetition-Suppressed Gene

See above.

(ii) Adipocyte Hyperplasia in Obesity

Rb-null (pRb−/−) preadipocytes show a higher proliferation rate compared to wild type. A study measured the percentage of pRb−/− 3T3 cells in S phase following five different treatments, cells grown in DMEM (asynchronous cells, marked A), cells grown to confluence in DMEM containing 10% calf serum and then maintained for 6 days in the same mixture (marked C), confluent cells split into subconfluent conditions (marked CR), confluent cells treated for 6 days with an adipocyte differentiating mixture (marked D), and differentiated cells split into subconfluent conditions (market DR). The results are summarized in FIG. 32 (Classon 2000⁴⁸⁶, FIG. 3A).

Asynchronous pRb(−/−) cells show a tendency for excessive cell replication. Moreover, pRb(−/−) differentiated cells show a higher probability for cell cycle re-entry. It should be emphasized that although pRb seems to affect the establishment of a permanent exit from cell cycle, pRb is not absolutely required since expression of C/EBPα and PPARγ bypasses the requirement for pRb and causes pRb(−/−) cells to differentiate into adipocytes (Classon 2000, FIG. 1B).

Transcription of the Rb gene increases with growth arrest and differentiation (see above). The relationship between pRb concentration and adipocyte differentiation was tested in a study that compared proliferative and differentiated brown (primary) and white (3T3-F442A) adipocytes in culture. The differentiation stage of the cells was determined following detection of lipid accumulation and expression of the specific differentiation markers aP2 and UCP-1. The results showed almost undetectable pRb levels in proliferative undifferentiated cells. On the other hand, pRb was clearly detected in nuclei of differentiated primary brown adipocytes (Puigserver 1998⁴⁸⁷, FIG. 2A) with lipid accumulation in their cytoplasm and UCP-1 expression (Ibid, FIG. 3) and in 3T3-F442A cells with lipid accumulation and aP2 expression. Moreover, Puigserver, et al., note that “the pRb levels measured by immunoblotting clearly increased during differentiation of 3T3 F442A cells (Ibid, FIG. 2B)” and that “there was an apparent positive correlation between pRb expression and lipid accumulation, since nuclei from cells with more lipid droplets in their cytoplasm were more strongly immunostained for pRb than those of cells with less lipid droplets (Ibid, FIG. 2A).”

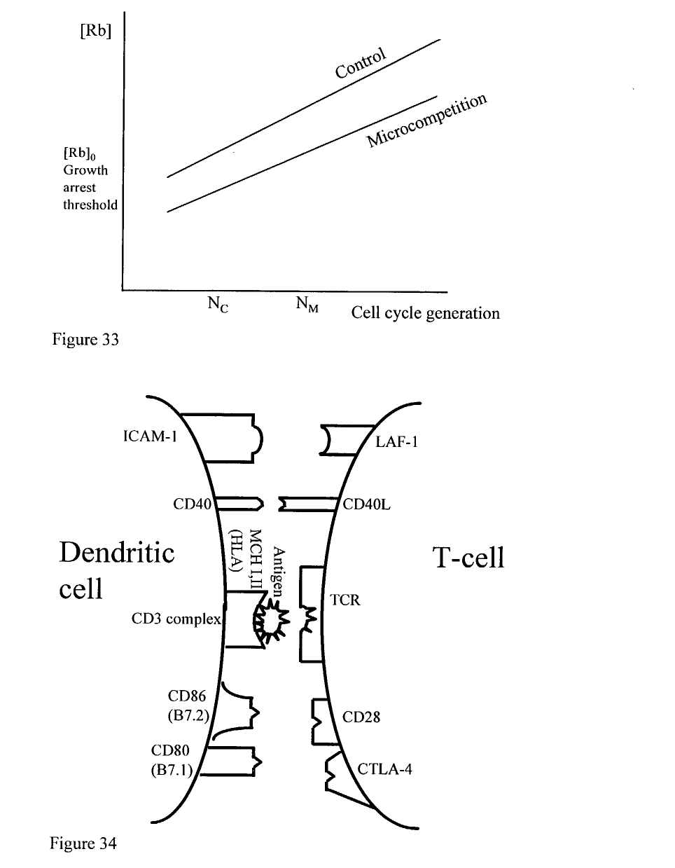

Richon, et al., (1992, ibid) proposed the following model for the relationship between Rb and growth arrest and differentiation (see also above). An inducer increases Rb transcription resulting in higher hypo- and total-pRb concentration. The increase in hypo-pRb prolongs G1. However, the initial increase in hypo-pRb is most likely not sufficient for permanent G1 arrest. Therefore, cells reenter cell cycle for a few more generations. While cells continue to divide, the increased rate of transcription results in hypo-pRb accumulation. When a critical hypo-pRb concentration, or threshold, is reached, the cells irreversibly commit to terminal differentiation. This model describes the determination of the commitment to differentiate as a stochastic process with progressive increases in the probability of G1/G0 arrest and differentiation established through successive cell divisions. Such a model would predict an increase in the number of cell cycle generations required for producing the threshold Rb concentration, under conditions of suppressed Rb transcription. Consider FIG. 33.

Microcompetition reduces Rb transcription. Therefore, the number of generations required to reach the required Rb concentration ([Rb]₀) under microcompetition (N_{M}) is greater than the number in controls (N_{C}). In obesity, therefore, one should observe excessive replication in vitro (Roncari 1986⁴⁸⁸, Roncari 1981⁴⁸⁹) and hyperplasia in vivo.

Returning to the non-obese HSL−/− mice (Osuga 2000, see above). Both HSL and Rb are microcompetition-suppressed genes. Therefore, both genes show reduced expression in obesity, resulting in adipocyte hypertrophy and hyperplasia. Since Rb transcription is most likely independent of HSL expression, pRb in HSL−/− mice is not under expressed and adipocytes in HSL−/− mice are not hyperplastic.

(4) Studies in Signaling

(a) Resistant ERK Agents in Obesity

The following are ERK agents showing cellular level or patient level resistance in obesity (for definition of cellular and patient level resistance and its relationship to microcompetition, see above).

(i) Oxytocin

The oxytocin receptor (OTR) is a GABP regulated gene (see above). Stock, et al., (1989⁴⁹⁰) tested whether plasma level of oxytocin is elevated in obese subjects, and if so, whether it is affected by weight reduction following gastric banding. Plasma levels of oxytocin were 4-fold higher in the obese subjects than in the control subjects. After the operation, oxytocin levels dropped dramatically, but were still markedly higher than control.

Moreover, obese pregnant women need more oxytocin stimulation of labor. Johnson, et al., found that, compared to a control group matched for age and parity, there was a significantly increased need for oxytocin stimulation of labor in obese patients weighing at least 113.6 kg (250 pounds) during pregnancy (Johnson 1987⁴⁹¹).

(ii) Zinc and Copper

Serum zinc, copper and magnesium levels were measured in healthy and obese children using atomic absorption spectrophotometry. Serum zinc and copper levels of obese children (mean value 102.40±2.78 micrograms/dL mean value 132.34±1.79 micrograms/dL, respectively) were markedly higher than control (mean value 80.49±2.98 micrograms/dL, and mean value 107.58±1.62 micrograms/dL, respectively). Serum copper concentrations were also significantly higher in obese children compared to healthy controls (Yakinci 1997⁴⁹²).

Serum zinc and copper levels were also determined in 140 diabetic patients and 162 healthy controls. A sub group of patients were classified as overweight (greater than 15% relative body weight). Obese patients showed a statistically singnificant increase in zinc levels while the copper level positively correlated with the zinc level (D'Ocon 1987⁴⁹³).

Taneja, et al., (1996⁴⁹⁴) measured the concentration of zinc in hair of obese men and women. The results showed a positive linear correlation between body weight, or body weight/height ratio, and hair zinc concentration. The correlation was stronger in men.

The following hormones and cytokines, which are all GABP kinase agents, also show resistance in obesity.

(iii) Insulin

Patients with non-insulin-dependent diabetes mellitus (NIDDM) and/or obesity generally suffer from insulin resistance (IR). Interestingly, most NIDDM patients are obese. Ludvik, et al., studied the effect of obesity and NIDDM on insulin resistance. Both lean NIDDM subjects and obese normal subjects were significantly insulin resistant compared to lean normal subjects (Ludvik 1995⁴⁹⁵).

Another study observed kinetic defects in insulin action in insulin resistant nondiabetic obese subjects. Insulin-stimulated glucose disposal was slower to activate and more rapidly deactivated in obese than in normal subjects. Oral glucose tolerance tests (OGTTs) were done in five controls and five obese subjects. While each of the control subjects had normal glucose tolerance, only two obese subjects tested normal for glucose tolerance. The remaining three obese subjects had impaired glucose tolerance. During the OGTT, both glucose and insulin levels were significantly higher in the obese subjects than the controls (Prager 1987⁴⁹⁶).

(iv) Leptin

The level of leptin in plasma increases with body weight (body mass index, BMI kg/m²). Plasma leptin levels are higher in females compared to males (Tasaka 1997⁴⁹⁷).

The ob/ob mouse has a mutated ob gene. The deficiency of leptin in the ob/ob mouse produces severe obesity. Contrary to the ob/ob mouse (and the db/db mouse with the mutated leptin receptor), in most obese humans the leptin and leptin receptors genes are normal. Moreover, except for some rare cases, the level of leptin in obese humans is elevated rather than reduced (Bjorbaek 1999⁴⁹⁸).

(v) Estrone, Estradiol

Urinary excretion of estrone (E1), estradiol (E2) and estriol (E3) was measured in obese post-menopausal women before and 6-12 months following participation in a weight loss program. Prior to the weight loss program, there was a significant correlation between estrone, weight and the Quetelet-index of obesity and between estriol and the Quetelet-index (de Waard 1982⁴⁹⁹).

Serum levels of sex hormones were studied in healthy, white postmenopausal women (mean age 58 years). Extraction, column chomatography, and radioimmunassay were used in combination to measure the serum concentrations of estrone, estradiol, testosterone, and androstenedione. Obesity was a major predictor of estrone and estradiol levels. Obese women had estrone levels 40% higher than nonobese women (Cauley 1989⁵⁰⁰).

In a subsequent study, Cauley, et al., (1994⁵⁰¹) compared sex steroid hormone levels between white and black women 65 years of age or older. The researchers used the same techniques to measure the serum levels of estrone, androstenedione, and testosterone as in the 1989 study. The results showed that black women had significantly higher serum estrone concentrations and markedly lower androstenedione levels compared to white women. There was a corresponding difference in the degree of obesity between the two groups.

(vi) Interleukin 1β (IL-1β)

Human coronary artery specimens from patients suffering from either coronary atherosclerosis or cardiomyopathy were studied for levels of IL-1β (Galea 1996⁵⁰²). The presence of IL-1β correlated with disease severity. The study discovered that IL-1β protein is elevated in the adventitial vessel walls of atherosclerotic coronary arteries compared to coronary arteries from nonischemic cardiomyopathic hearts.

Serum IL-1β levels were also determined in patients with ischaemic heart disease. The results showed that the mean serum IL-1β concentrations were higher in patients with ischaemic heart disease, in particular in those with minimal coronary artery disease and angina (Hasdai 1996⁵⁰³).

(vii) Interleukin 6 (IL-6)

Type II diabetes mellitus (non-insulin-dependent diabetes mellitus, NIDDM) is assoicated with increased blood concentrations of markers of the acute-phase response, including interleukin-6. The combination of hypertriglyceridaemia, low serum HDL-cholesterol concentrations, hypertension, obesity and accelerated atherosclerosis, termed metabolic syndrome X, is often associated with NIDDM. To investigate this association, two groups of Caucasian NIDDM patients were studied. The first group, with any 4 or 5 features of syndrome X, was compared with the second group, with 0 or 1 feature of syndrome X. The groups were matched for age, sex, diabetes duration, glycaemic control, and diabetes treatment. Age and sex matched healthy non-diabetic subjects were controls. The results showed a marked increase in serum IL-6 between the three groups. The lowest levels were found in non-diabetic subjects, intermediate levels in NIDDM patients with 0 or 1 feature of syndrome X and the highest levels in NIDDM patients with a 4 or 5 features (Pickup 1997⁵⁰⁴, Pickup 1998⁵⁰⁵).

(viii) Tumor Necrosis Factor α (TNFα)

Sixty five patients were tested for TNFα levels. The majority of the patients had android obesity, elevated leptin, insulin resistant, coronarographically confirmed microvascular angina pectoris or IHD. Most of the patients suffered from a myocardial infarction with one or more significant stenoses on the epicardial coronary arteries. Fifty percent of the patients had elevated TNFα, and 28% elevated IL-6 (Hrnciar 1999⁵⁰⁶).

(b) Non Resistant ERK Agents in Obesity

Some GABP kinase agents show no resistance. Consider the following cases.

(i) Interleukin 2β (IL-2β)

IL-2β is an ERK agent with the receptors, interleukin 2 receptor β chain (IL-2Rβ) and IL-2 receptor γ-chain (γc). Both receptors are stimulated by GABP (Markiewicz 1996, ibid, Lin 1993, ibid). Microcompetition for GABP reduces transcription of the receptors. Since any control in this pathway has to be downstream from the receptors, microcompeition for GABP diminshes expression of the control. The reduced expression of the control reduces its repressive effect on IL-2β, which elevates the concentration of IL-2β. However, IL-2β itself is a GABP stimulated gene (Avots 1997, ibid). Therefore, microcompetition also reduces transcription of IL-2β. The combined effect of diminished repression on transcription and diminished transactivation of transcription can result in a decline, increase, or no change in the concentration of IL-2β in obesity.

(ii) GM-CSF

Granulocyte-macrophage colony stimulating factor (GM-CSF) is an ERK agent. One study showed that GM-CSF (20 ng/ml) significantly inhibited neutrophil apoptosis. The inhibition of apoptosis was significantly attenuated by PD98059, an MEK1 specific inhibitor (Klein 2000⁵⁰⁷). Another study showed that bone marrow-derived macrophages proliferate in response to GM-CSF. The MEK1 specific inhibitor, PD98059, blocked the GM-CSF stimulated cell proliferation. Moreover, this study showed the time-course of ERK activation by GM-CSF, where maximal activation occurred 5 min after stimulation (Valledor 2000⁵⁰⁸).

As a GABP kinase agent one would expect to observe resistance in obesity and obesity related disease. However, the GM-CSF gene is transactivated by etsl (Thomas 1997⁵⁰⁹). Therefore, microcompetition for ets 1 can result in either a decline, increase or no change in GM-CSF concentration in obesity and obesity related diseases.

(5) Studies with Viruses

Until recently, the relationship between viral infection and human obesity has been completely ignored.

(a) Human Adenovirus 36 (Ad-36)

A recent study inoculated chickens and mice with human adenovirus Ad-36. Weight matched groups were inoculated with tissue culture media as non-infected controls. Ad-36 inoculated and uninfected control groups were housed in separate rooms under biosafety level 2 or better containment. The chicken study was repeated three times. The first chicken experiment included an additional weight matched group of chickens that was inoculated with CELO (chick embryo lethal orphan virus), an avian adenovirus. Food intake and body weight were measured weekly. At the time of sacrifice blood was drawn and visceral fat was separated and weighed. Total body fat was determined by chemical extraction of carcass fat. In experiment 1, the results showed that the visceral fat of the Ad-36 chickens was 100% greater that controls (Dhurandhar 2000⁵¹⁰, Table 1), in experiment 2, visceral fat was 128% greater than controls (Ibid, Table 3), in experiment 3, visceral fat was 74% greater than control (Ibid, Table 4). In all three experiments there was no difference in food intake or body weight between Ad-36 chickens and controls. Chickens inoculated with CELO virus showed no change in visceral fat. The Ad-36 mice visceral fat was 67% greater than controls and mean body weight was 9% greater. There was no difference in food intake. Sections of the brain and hypothalamus of Ad-36 inoculated animals showed no overt histopathological changes. Ad-36 DNA could be detected in adipose tissue, but not skeletal muscles of randomly selected animals for as long as 16 weeks after Ad-36 inoculation. Based on these results Dhurandhar concluded that “the role of viral disease in the etiology of human obesity must be considered.”

(b) HIV

Recently, several studies documented a new syndrome associated with HIV infection termed “lipodystophy,” or “fat redistribution syndrome” (FRS). The symptoms typical of FRS, such as peripheral lipodystrophy, central adiposity, hyperlipidemia and insulin resistance (for a recent review see Behrens 2000⁵¹¹), are similar to syndrome X symptoms (Engelson 1999⁵¹²) (Syndrome X is also known as “insulin resistance” or plain “obesity.”) The cause of FRS is unknown. The temporal association between the recognition of FRS and the application of protease inhibitor therapy has led several investigators to conclude that FRS is a result of protease inhibitor therapy. However, since FRS was also identified in HIV-infected patients who were not taking protease inhibitors, other researchers concluded that FRS might be a characteristic of the HIV infection, only unmasked by prolonged survival associated with protease inhibitors treatment.

HIV is a GABP virus. HIV infection results in microcompetition between virus and the host, which leads to obesity. (Moreover, recent studies report that HIV infection is assoicated with a greater risk of developing atherosclerosis and diabetes mellitus. Atherosclerosis and diabetes mellitus are another two diseases caused by microcompetition.)

(6) Other Foreign Polynucleotide-Type Disruptions and Obesity

(a) Hypothesis: Genetic Mutation, Injury, Diet or a Weak ERK Signal

A genetic mutation, injury or diet can result in a deficiency in an ERK agent or ERK receptor. Such deficiency produces a weak ERK signal. A weak ERK signal disrupts the GABP pathway, and therefore, induces clinical symptoms similar to the symptoms resulting by microcompetition between cellular genes and a foreign polynucleotide for GABP.

(b) EXAMPLES

(i) Leptin

Homozygous mutations in genes encoding leptin or the leptin receptor lead to early-onset obesity and hyperphagia (Clement 1998⁵¹³). For instance, mutation in the ob (leptin) gene is associated with obesity in the ob/ob mouse.

Obesity in the db/db mouse is associated with mutations in the db (leptin receptor) gene. An alternatively spliced transcript of the leptin receptor encodes a form with a long intracellular domain. The db/db mouse produces this alternatively spliced transcript with a 106-nucleotide insertion that prematurely terminates the intracellular domain. Moreover, the db/db mouse also exhibits a point mutation (G→T) in the same gene. The long intracellular domain form of the receptor participates in signal transduction and the inability to produce the long form in db/db mice contributes to their extreme obese phenotype (Chen 1996⁵¹⁴).

Obesity in the Zucker fatty (fa/fa) rats is associated with mutations in the fa gene which encodes a leptin recpetor. The fa mutation is a missense mutation (269 gln→pro) in the extracellular domain of the leptin receptor. This mutation causes a decrease in cell-surface expression, a decrease in leptin binding affinity, defective signaling to the JAK-STAT pathway and reduced ability to activate transcription of the egrl promoter (de Silva 1998⁵¹⁵). Yamashita, et al., found that by binding to the long form of its receptor, leptin increased the tyrosine phosphorylation of STAT3 and ERK in Chinese hamster ovary (CHO) cells. In CHO cells with a fa mutated receptor, the leptin induced phosphorylation of both STAT3 and ERK was lower (Yamashita 1998⁵¹⁶).

ERK Complements

Let A and B be two ERK agents. Assume that A is not an ERK receptor for B. Administration of B can alleviate the symptoms associated with a deficiency in A or an ERK receptor for A.

If A is not an ERK receptor for B, B will be called an “ERK Complement” for A. Notice that the relationship is asymmetric. If B is downstream from A, B is an ERK complement for A, while A is not an ERK complement for B.

IL-1β as ERK Complement for Leptin

A low dose injection of human recombinant IL-1β to gentically obese ob/ob and db/db mice normalized glucose blood levels for several hours (del Rey 1989⁵¹⁷). In another study, chronic intracerebroventricular (ICV) microinjection of IL-1β to obese (fa/fa) Zucker rats caused a 66.1% decrease in nighttime food intake (Ilyin 1996⁵¹⁸).

Luheshi, et al., (1999⁵¹⁹) showed that IL-1β is an ERK receptor for leptin. However, IL-1β can still be as ERK complement for leptin if leptin is not a receptor for IL-1β (asymmetry of the complement condition).

TNFα as ERK Complement for Leptin

ICV microinjection of TNFα (50, 100 and 500 ng/rat) to obese (fa/fa) Zucker rats in triplicate decreased short-term feeding (4 hours) by 17%, 20%, and 20%, nighttime feeding (12 hours) by 13%, 14% and 13% and total daily food intake by 11%, 12% and 11%, respectively (Plata Salaman 1997⁵²⁰).

LPS as ERK Complement for Leptin

Administration of LPS (0.1, 1, 10, 100 μg) to db/db mice induced a significant decrease in food intake (25%, 40%, 60%, 85%, respectively, in the first 24 hours post injection). The effect on ob/ob mice was similar (Faggioni 1997⁵²¹).

(ii) Insulin

A mutation in the insulin receptor substrate-1 (IRS-1) is a risk factor for coronary artery disease (CAD). Insulin resistance is correlated with a higher risk of atherosclerosis. Insulin receptor substrate-1 (IRS-1) is a key component of tissue insulin sensitivity. A mutation (G972R) of the IRS-1 gene, which reduces IRS-1 function and has been connected to decreased sensitivity to insulin, was studied to see if it had any role in predisposing individuals to coronary artery disease (CAD). In this study, CAD patients had a much higher incidence of the mutation than the control group (18.9% versus 6.8%, respectively). The relative risk of CAD associated with the mutation increased in the obese patients and patients with a cluster of abnormalities of insulin resistance syndrome. These results indicate that the G972R mutation in the IRS-1 gene is a strong independent predictor of CAD. In addition, this mutation significantly enhanced the risk of CAD in both obese patients and in patients with clinical features of the insulin resistance syndrome (Baroni 1999⁵²²).

(iii) Transforming Growth Factor-β (TGFβ)

Mutations in the TGFβ receptor type II gene are associated with various cancers. Several human gastric cancer cell lines were studied for genetic abnormalities in the TGFβ type II receptor gene. Deletion of the type II receptor gene in two of eight cell lines, and amplification of the gene in another two lines, was detected in Southern blots. Other abnormalities in the gastric cancer cells resistant to the growth inhibitory effect of TGFβ included expression of either truncated or undetectable TGFβ type II receptor mRNAs. The one cell line not resistant to the growth inhibitory effect of TGFβ showed no abnormalities in type II receptor gene (Park 1994⁵²³). Mutation of the TGFβ receptor type II gene is characteristic of colon cancers with microsatellite instability or replication errors (RER+). Specific mutations in a polyadenine repeat of the TGFβ type II receptor gene are common in both RER+ colon cancers and RER+ gastric cancers (Myeroff 1995⁵²⁴).

Mutations in the TGFβ receptor type II gene are also associated with atherosclerosis. High fidelity PCR and restriction analysis was adapted to analyze deletions in an A10 microsatellite within the TGFβ receptor type II gene. DNA from human atherosclerotic lesions, and cells grown from lesions, showed acquired 1 and 2 bp deletions in TGFβ receptor type II gene. The mutations could be identified within specific patches of the lesion, while surrounding tissue, or unaffected arteries, exhibited the wild-type genotype. This deletion causes loss of receptor function, and thus, resistance to the antiproliferative and apoptotic effects of TGFβ1 (McCaffrey 1997⁵²⁵).

A deficiency in the TGFβ receptor type II gene causes osteoarthritis. An overexpressed TGFβ cytoplasmically truncated type II receptor competes with the cellular receptors for complex formation, thereby acting as a dominant-negative mutant receptor. Transgenic mice expressing the dominant-negative mutant receptor in skeletal tissue developed progressive skeletal degeneration. The pathology strongly resembled human osteoarthritis. This controled expriment in mice shows that a weak TGFβ signal leads to the development of degenerative joint disease similar to osteoarthritis in humans (Serra 1997⁵²⁶).

(iv) Estrone and Estradiol

The ovaries in polycystic ovary syndrome (PCOS) produce less estradiol in response to follicle-stimulating hormone (Caruso 1993⁵²⁷). PCOS is associated with high blood pressure, hyperinsulimia, insulin resistance and obesity.

Ovariectomy reduces the concentration of estradiol, sometimes to undetectable levels (Wronski 1987⁵²⁸). Ovariectomy is also associated with obesity.

(v) Zinc and Copper

Singh, et al., (1998⁵²⁹) surveyed 3,575 subjects, aged 25 to 64 years. The results showed that the prevalence of coronary artery disease (CAD), diabetes and glucose intolerance is associated with lower intake of dietary zinc. In addition, hypertension, hypertriglyceridemia and low high-density lipoprotein cholesterol levels increased as zinc intake decreased.

(vi) Metallothionein-Null

Metallothionein is a receptor of the ERK agent zinc. After weaning, MT-null mice consumed more food and gained more weight at a more rapid rate than control mice. The majority of the adult male mice in the MT-null colony showed moderate obesity (Beattie 1998, ibid).

(vii) CD18-null

Chinese hamster ovary (CHO) fibroblast cell lines were engineered to express the CD11a/CD18 or CD11b/CD18 antigens. These cell lines were induced with LPS. Otherwise LPS-nonresponsive fibroblasts became responsive to LPS upon heterologous expression of CD11a/CD18 and CD11b/CD18 (Flaherty 1997⁵³⁰). CD11c/CD18 also activated cells after binding to LPS (Ingalls 1995⁵³¹). In another study, both wild type CD11b/CD18 and mutant CD11b/CD18 lacking the cytoplasmic domains still transmitted a signal in response to LPS (Ingalls 1997⁵³²). Although full length CD11b/CD18 is needed for productive phagocytic signals, LPS activation does not require the cytoplasmic domains. Perhaps CD11b/CD18 activates cells by presenting LPS to a downstream signal transducer (Ingalls 1997). These studies indicate that CD11a/CD18 and CD11b/CD18 are receptors of the ERK agent LPS.

CD11a/CD18 binds the intercellular adhesion molecule-1 (ICAM-1). ICAM-1 null mice (ICAM-1−/−) gained more weight than control mice after 16 weeks of age, and eventually became obese despite no obvious increase in food intake. ICAM-1−/− mice also showed an increase susceptibility to develope obesity under a high fat diet.

CD11b/CD18 binds macrophage 1 (MAC-1). MAC-1 null mice (MAC-1−/−) were also susceptible to diet-induced obesity, and exhibited a strong similarity in weight gain with sex-matched ICAM-1-1-mice (Dong 1997, ibid).

f) Stroke

(1) Introduction

Stroke (cerebrovascular accident, CVA) is cardiovascular disease resulting from disrupted blood flow to the brain due to occlusion of a blood vessel (ischemic stroke) or rupture of a blood vessel (hemorrhagic stroke). Interruption in blood flow deprives the brain of oxygen and nutrients, resulting in cell injury in affected vascular area of the brain. Cell injury leads to impaired or lost function of body parts controlled by the injured cells. Such impairment is usually manifested as paralysis, speech and sensory problems, memory and reasoning deficits, coma, and possibly death.

Two types of ischemic strokes, cerebral thrombosis and cerebral embolism, are most common accounting for about 70-80 percent of all strokes. Cerebral thrombosis, the most common type of stroke, occurs when a blood clot (thrombus) forms blocking blood flow in an artery supplying blood the brain. Cerebral embolism occurs when a wandering clot (an embolus) or another particle forms in a blood vessel away from the brain, usually in the heart. The bloodstream carries the clot until it lodges in an artery supplying blood to the brain blocking the flow of blood.

(2) Microcompetition and Stroke

Microcompetition causes atherosclerosis. Like coronary artery occlusion, atherosclerosis in arteries leading blood to the brain (such as carotid artery) or in the brain may result in arterial occlusion through plaque formation or plaque rupture and in situ formation of a thrombus (see chapter on atherosclerosis above). Lammie (1999⁵³³) reports observations supporting similar pathogenesis in coronary artery disease (CAD) and stroke. In general, numerous studies report the association between atherosclerosis and stroke (see, for instance, Chambless 2000⁵³⁴, O'Leary 1999⁵³⁵).

In addition, microcompetition increases TF expression on circulating monocytes. Monocytes originate from CD34+ progenitor cells (Hart 1997⁵³⁶, FIG. 3). CD34+ cells are permissive for a GABP viral infection. For instance, Zhuravskaya, et al., (1997⁵³⁷) demonstrated that human cytomegalovirus (HCMV), a GABP virus, persisted in infected bone marrow (BM) CD34+ cells (see also, Maciejewski and St Jeor 1999⁵³⁸, Sindre 1996⁵³⁹). The infection of CD34+ with a GABP virus increases TF expression on circulating monocytes. Such excessive TF expression in stroke patients was documented in a few studies (see, for instance, Kappelmayer 1998⁵⁴⁰). The excessive TF expression increases the probability of coagulation and formation of an embolus.

(g) Autoimmune Disease

(1) Conceptual Building Blocks

(a) T-Cell Deletion vs. Retention and Th1 vs. Th2 Differentiation

Dendritic cells (DC) and macrophages are professional antigen presenting cells (professional APC). For simplicity, the text uses the symbol DC to represent both types of professional APC.

DC bind T cells. FIG. 34 illustrates some of the molecules on the surface of DC and T cells participating in this binding.

Strength of DC and T-cell binding, denoted [DC·T], is a positive function of B7 concentration on surface of DC, denoted [B7], a negative function of CTLA4Ig concentration on surface of T-cell, denoted [CTLA4Ig], and a positive function of concentration of the major histocompatibilty complex (MHC) bound to antigen on DC, denoted [Ag]. The following formula presents these relationships.

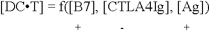

A (+) sign under [B7] means a positive relationship, that is, an increase in B7 surface concentration increases the strength of DC and T-cell binding. A (−) sign under a variable indicates a negative relationship.

We assume a greater than zero rate of substitution between [B7] and [Ag], that is, increase in [B7] can compensate, to a certain degree, for decrease in [Ag], and vice versa. [DC·T] determines CD8+ retension vs. deletion and Th1 vs. Th2 differentiation.

(i) Increase in [DC·T] Increases the Probability of Peripheral CD8+ Retension vs. Deletion

Low [DC·T] leads to peripheral CD8+ proliferation and deletion. The deletion is specific for the antigen presented on MHC. High [DC·T] results in peripheral CD8+ proliferation and retention. T-cells do not differentiate between self or foreign antigen. They respond only to [DC·T].

Define antigen specific peripheral tolerance as deletion of T-cells specific for this antigen. Using this term, it can be said that low [DC·T] induces tolerance.

(ii) Increase in [DC·T] Increases the Probability of Th1 vs. Th2 Differentiation

T helper lymphocytes can be divided into two subsets of effector cells based on their function and the cytokines they produce. The Th1 subset of CD4+ T cells secretes cytokines usually associated with inflammation, such as interleukin 2 (IL-2), interleukin 12 (IL-12), interferon γ (IFNγ) and Tumor necrosis factor β (TNFβ), and induces cell-mediated immune responses. The Th2 subset produces cytokines such as interleukin 4 (IL-4), interleukin 5 (IL-5), interleukin 6 (IL-6), interleukin 10 (IL-10), and interleukin 13 (IL-13, which help B cells to proliferate and differentiate and is associated with humoral-type immune responses (see recent review Constant 1997⁵⁴¹).

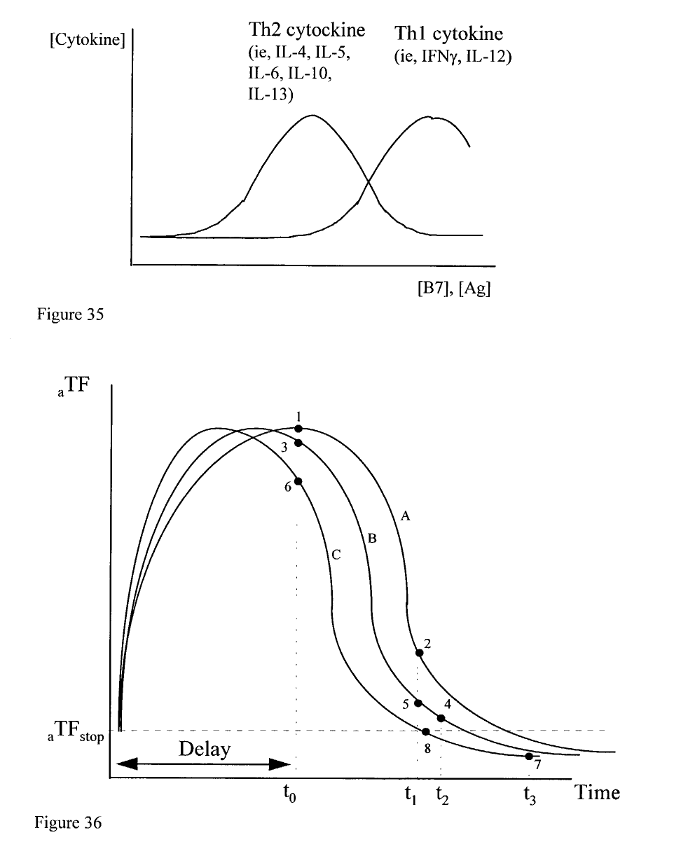

In relevant physiological conditions, low [DC·T] induces CD4+ differentiation into Th2 while high [DC·T] induces Th1 differentiation. [B7] and [Ag] increase [DCT] (see formula above). Therefore, increase in either [B7] or [Ag], increases the probability of Th1 vs. Th2 differentiation. This concenpt is represented in FIG. 35.

The results in Rogers and Croft (1999⁵⁴²) support such a relationship. Naive CD4 cells were stimulated with varying doses of moth cytochrome c (MCC) presented on splenic APC and cultured for 4 or 12 days. An equivalent number of surviving T cells was restimulated with a single dose of Ag and assayed for secretion of Th1 and Th2 cytokines. The results showed that the length of differentiation period (4 or 12 days) affects the cytokine profile induced by varying doses of native peptide (Rogers and Croft 1999, ibid). Overall, after 12 days of differentiation, lower doses of high affinity peptides produced T-cells mostly secreting Th2 cytokines. In contrast, higher doses of high affinity peptides resulted in more T-cells secreting Th1 cytokines. Roger and Croft summarized these, and other results, in a figure (Ibid, FIG. 7) almost identical to the figure above. (The figure for T-cells after 4 days in culture is different. However, since autoimmune disease is a chronic condition, extended exposure to APC seem to be a better description of the CD4+ T-cells in vivo environment).

(b) Increase in Probability of Antigen Internalization Increases [Ag] and [B7]

An antigen is a molecule that induces an internalization response in DC (phagocytosis, cell engulfinent, etc). Cell debris, apoptotic cells, foreign proteins, etc. are antigens, that is, activate an internalization response by DC.

An increase in the concentration of internalized antigens stimulates antigen processing and presentation on DC surface, or [Ag]. The increase in the concentration of internalized antigens also increases [B7], or costimulation (see, for instance, Rovere 2000⁵⁴³ and Rovere 1998⁵⁴⁴ for observation consistent with this concept).

Consider a stationary DC. Increase in antigen concentration in the DC environment increases the DC probability of antigen internalization. Consider a DC migrating through an environment with fixed antigen concentration. Slower DC migration increases the DC probability of antigen internalization. Therefore, both increase in antigen concentration in the cell environment, and decrease in the cell migration speed, increase [Ag] and [B7].

Assume an increase in concentration of internalized antigens decreases cell migration speed. The decrease in migration speed amplifies a small increases in antigen concentration in the DC environment into a large increases in [Ag] and [B7]. Such amplification increases the sensitivity of DC to its environment.

(c) Chemokines Carry a Homing Signal for T-Cells and Circulating Professional APCs

A source DC releases chemokines. The chemokines direct activated T-cell and more DC to the source. The steering of T-cells and new DC is most effective when the source DC is stationary (otherwise, T-cells and new DC need to chase a moving target). Some of the chemokines secreted by DC are RANTES (regulated upon activation, normal T cell expressed and secreted), MIP-1α, MIP-1β (macrophage-inflammatory protein-1α and 1β). CCR5 is a receptor for these chemokines variably expressed on monocytes, activated T cells, natural killer cells, and dendritic cells.

(d) Cytotoxic T Lymphocytes (C TL)

Assume a stationery source DC releasing chemokines. Antigen specific CTL enter the tissue near the stationary DC and bind and destroy all target cells, that is, cells which present the specific antigen on their MHC. The target cells include the stationary DC and all tissue cells which present the antigen.

(2) Model

Damaged tissue is defined as tissue showing abnormal morphology. Tolerance, activation and autoimmune disease are defined as immune dynamics which result in no tissue damage, reversible, or self correcting tissue damage, and irreversible tissue damage, respectively. Note that these definitions are different from acute vs. chronic immune activation.

The following sections present a model that describes the conditions inducing tolerance, activation and autoimmune disease.

(a) Tolerance

Tolerance is defined as immune dynamics that result in no tissue damage. Consider the following dynamics.

Terminology: In the atherosclerosis chapter foam cell migration back to circulation was called backward motility. Since backward motility essentially means out of tissue migration, this text uses the same term to describe DC migration from tissue to lymph vessel.

DC continuously enter tissues. In tissue, the cells collect, process and present antigens on MHC. Internalized antigens induce oxidative stress, which decreases binding of GABP to the tissue factor (TF) promoter, resulting in increased TF expression (see effect of GABP on TF expression above in atherosclerosis chapter). TF propels DC backward motility, which is migration out of tissue and into a lymph vessel. Since backward motility takes a relatively short time, the DC entering the lymph vessel show only a small increase in [B7]. Moreover, under normal conditions, the concentration of antigens in the DC migration path is low. As a result, the DC entering the lymph vessel also show low [Ag]. In the draining lymph node, DC bind naive T-cells expressing T-cell receptors (TCR), which match the presented antigens. Since [B7] and [Ag] are low, [DC·T] is low (see formula above). As a result, the bound T-cells proliferate and die.

(b) Immune Activation

Activation is defined as immune dynamics that result in reversible tissue damage.

(i) The “Slow DC” Model

Consider a tissue with excessive local production of an antigen. For simplicity, let the antigen originate from a single cell, called the origin. Antigen concentration near the origin is not uniform. Some regions contain “normal,” or low, concentrations, other contain moderate concentrations, yet other contain high antigen concentrations. Consider three dendritic cells DC_{A}, DC_{B} and DC_{C}. DC_{A}, DC_{B} and DC_{C} migrate through the regions of “normal,” moderate, and high antigen concentrations, respectively. Higher antigen concentration results in higher rate of antigen internalization, faster increase in cellular free radicals and faster increase in TF expression (oxidative stress reduces the binding of GABP to the TF gene and increases its transcription, see above). Consider FIG. 36.

TF activity, marked ₐTF, is a function of surface TF concentration. A faster increase in TF concentration moves the ₐTF graph to the left (the atherosclerosis chapter discusses the shape of the ₐTF curve and the relationship between ₐTF and TF surface concentration). Assume the speed of DC migration in tissue can be represented as a linear function of ₐTF. Then, the distance traveled by a DC is equal to the integral of its ₐTF function from to, the time the cell starts backward motility, to the time the DC leaves the tissue and enters a lymphatic vessel. Consider DC_{A}. DC_{A} starts to migrate at time to and at time t₁ (point 2) reaches the lymphatic vessel. The area under curve A from point 1 to point 2 is equal to the distance traveled by DC_{A}. Mark this area with

$${\int }_{t0}^{t1} A ⅆ x .$$

Consider DC_{B}. Curve B represents a faster increase in TF concentration on the DC. To reach the lymphatic vessel, DC_{B} must travel the same distance as DC_{A}. However, DC_{B} needs a longer time to travel this distance, or t₂>t₁. At time t1, the distance traveled by DC_{B} is represented by the area under curve B defined by points 3 and 5, or

$${\int }_{t0}^{t1} B ⅆ x .$$

This area is only part of the area under curve A defined by points 1 and 2, in symbols,

$${\int }_{t0}^{t1} B ⅆ x < {\int }_{t0}^{t1} A ⅆ x .$$

To increase the area, or distance travels by DC_{B}, t₂ must be greater than t1. See the area defined by points 3 and 4 in the figure. Does every DC reach the lymphatic vessel? To answer this question, assume that every ₐTF greater than ₐTFₛₜₒₚ propels migration. DC_{B} spends a longer time migrating, however, the cell reaches the lymphatic vessel. In comparison, to successfully reach the lymphatic vessel, DC_{C} must spend an even longer time on the (same) road. In the figure, this time is marked by t₃. This extra time is prevents the cell from reaching the lymphatic vessel. According to the figure, to reach the lymphatic vessel, DC_{C} depends on TF activities that no longer propel migration. All ₐTF between points 8 and 7 are bellow ₐTFₛₜₒₚ. DC_{C} ends up trapped in tissue. Moreover, the higher the concentration of antigen in the DC environment, the less is the distance traveled by the cell, and the nearer the cell's final resting site relative to the origin.

(ii) The “Two-Peak” System

Consider insulin producing β cells as an example for tissue in the “slow DC” model above. Assume β cells are induced to increase their production of antigens, resulting in an increase in the concentration of antigens in the DC migratory path. Such an increase might result from injury, infection, transgene expression, etc (see examples below). Since, in most cases, antigen production involves apoptosis, we call this initial event “trigger apoptosis.” For simplicity, let trigger apoptosis is self limiting. The curve illustrating the number of apoptotic β cells over time is bell shaped (see following figure). If we assume that every β cell produces the same concentration of antigens, this curve can also represent the antigen concentrations in the DC environment.

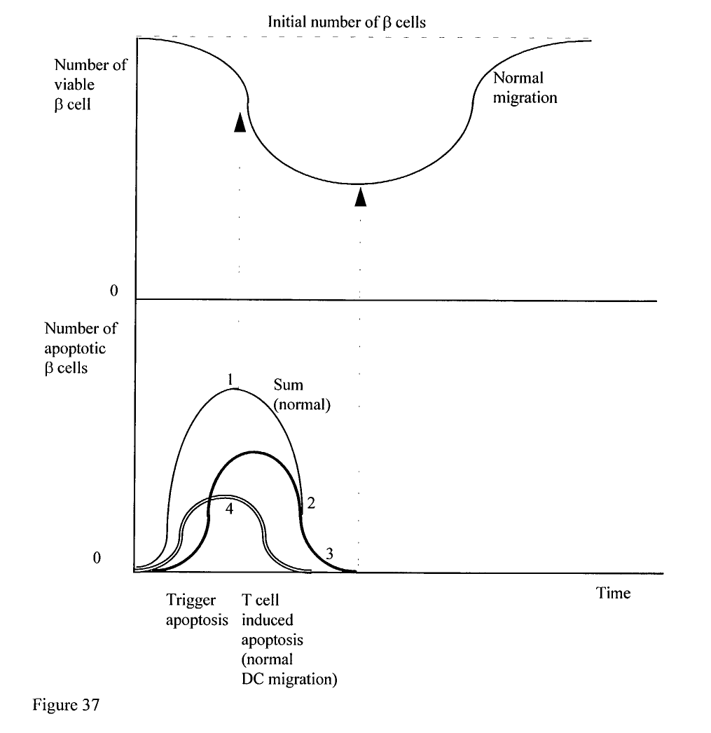

DC continuously migrate through the pancreas. As a result of the excessive production of autoantigen, some DC internalize more antigens and begin to slow down, which further increases their antigen internalization. A few slower migrating DC reach the lymph vessel (DC_{B} above), and than the draining lymph node where they present higher [Ag] and [B7] to T-cells inducing proliferation and retention. Other slower migrating DC end up trapped in the tissue (DC_{C} above). These cells release chemokines that direct activated T-cells to the site of excessive antigen production. The chemokines also direct more DC to the same site, which amplifies the initial reaction. Infiltrating T-cell bind trapped DC and β cells inducing a second wave of apoptosis. The T-cell induced apoptosis decreases the number of trapped DC, the production of DC chemokines, the infiltration of T-cells and new DC, returning immune dynamics to tolerance. Since the T-cell induce apoptosis is self-limiting, it is represented in FIG. 37 with a bell shape curve.

Overall, the number of viable β cells is equal the initial number of β cells minus the total number of apoptotic cells (initial number of β cells—trigger apoptosis—T-cell induced apoptosis). In FIG. 37, the sum of apoptotic cells is represented by the curve 0, 1, 2, 3 and the corresponding “number of viable β cells” curve is illustrated in the top half of the figure. Note that the peak of the “sum curve” corresponds to the turn in the S shape of the “number of viable β cells” curve, and the end of the “sum curve” corresponds to the minimum point on the “number of viable β cells” curve (see dotted arrows). The right hand side of the “number of viable β cells” curve illustrates β cell neogenesis. Note that the final number of viable β cell is equal the initial number, and therefore, at termination tissue damage is reversed.

(iii) The “Two-Peak” Dynamics

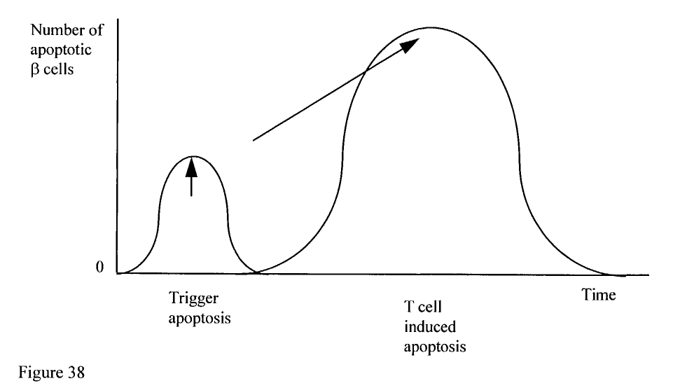

Assume an increase in trigger apoptosis. How does the two-peak system respond to such a change? Consider FIG. 38.

The increase in trigger apoptosis produces more antigens. DC internalize more antigens. The excess oxidative stress increases TF surface expression. DC migration to the lymph node is slower, and therefore, T-cell activation is delayed. However, when DC eventually reach the lymph node, they present higher [Ag] and [B7], and, therefore, activate more T-cells (higher probability for activation and retention rather than activation and deletion). Moreover, more DC are trapped in the tissue. These cells produce more chemokines and chemoattract more T-cell which infiltrate the tissue producing higher rate of apoptosis. Overall, the increase in trigger apoptosis shifts the second peak right and up.

(c) Autoimmune Disease

(i) The “Excessively Slow DC” Model

Autoimmune disease is defined as immune dynamics that produce irreversible tissue damage, or abnormal tissue morphology.

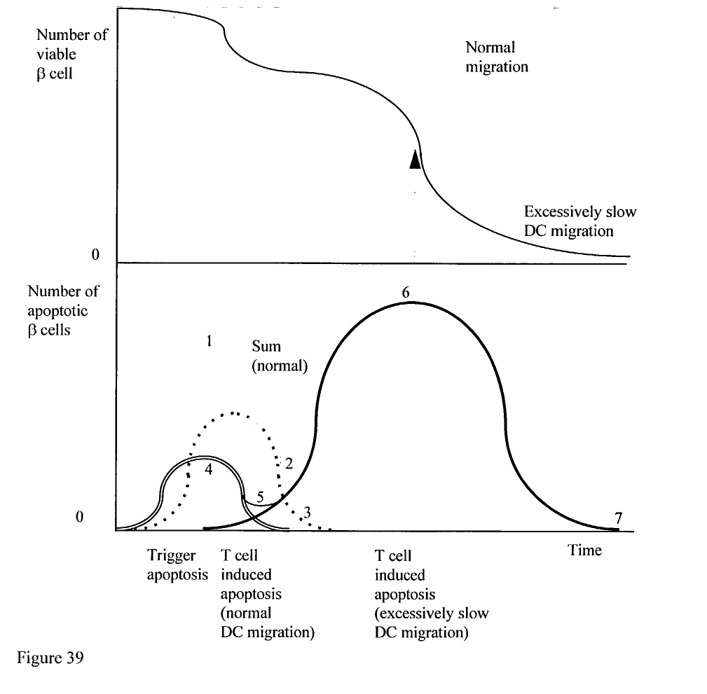

Consider a situation where an exogenous disruption (local of systematic) slows DC backward motility. These DC will be called “excessively slow.” Since TF propels backward motility, a disruption which decreases or increases TF surface concentration (both directions have the similar effects because of TF encryption, see the atherosclerosis chapter above) can produce excessively slow DC. Consider FIG. 39.

The disruption shifts the second peak to the right. As with increased trigger apoptosis, DC migration to the lymph node is slower, which result in a shift of the second peak right and up. The sum of β cell apoptosis in this case is represented by the two-peak curve (0, 4, 5, 6, 7). The question is what is the shape of the corresponding “number of viable 1 cells” curve? Excessive β cell apoptosis induces excessive tissue damage. If tissue regeneration capacity is limited, there exist a level of β cell apoptosis which result in permanent reduction in the number of viable β cells. Note that in the figure above, the corresponding “number of viable β cells” curve shows complete destruction of β cells. Under limited regeneration capacity, such damage is irreversible, and, therefore, describes autoimmune disease.

(3) Predictions and Evidence

The studies described in the following section use different interventions. In terms of the two-peak model, these interventions decrease or increase trigger apoptosis, excessively slow DC backward motility, etc. The following sections compare the predicted effects of such interventions with the actual reported responses.

(a) Animal Models

The expression of cellular molecule, M, in a tissue cell C (C is not a DC) is called “excessively high” if the normal process of antigen production in C causes autoimmune disease.

Some transgenic animals are designed to express a foreign gene (see examples below). Since cells show variable transgene expression, it likely that some cells show high transgene expression (and others low expression). Since excessively high transgene expression produces an autoimmune disease, we call the cells with high transgene expression “immune susceptible cells.” The situation of autoimmune disease in transgenic animals without further intervention is sometimes called “spontaneous” (see examples below). Using this term, it can be said that, in transgenic animals, the immune susceptible cells show a high probability for spontaneous destruction.

(i) Tolerance Dymanics

A recent review summarizes many observations relating to issues of ignorance and tolerance (Heath 1998⁵⁴⁵). Based on these observations Heath, et al., concluded that “taken together, there is compelling evidence that in order to maintain self-tolerance a specialized APC is capable of capturing tissue antigens, transporting them to the lymphoid compartment, i.e., the draining lymph nodes, and presenting them to both naive CD4+ and CD8+ T cells . . . . This APC appears to be capable of processing exogenous antigens into class I and class II pathways . . . . The above data argue for the existence of a “professional” APC that constitutively induces tolerance to antigens expressed in extralymphoid tissues . . . . In studies using transgenic mice expressing different levels of OVA in the pancreas, we have recently found that antigen concentration is critical in determining whether such antigens are cross-presented in the draining lymph nodes. The level of antigen expression appears to determine whether an antigen induces cross-tolerance or is ignored by naive T cells . . . . It is interesting to note that deletion of both CD4+ and CD8+ T cells is preceded by a period of proliferation, suggesting that the APC responsible for tolerance induction must be capable of activating T cells into proliferative cycles. Moreover, the APC is a cell capable of trafficking from peripheral tissues to draining lymph node. Even more importantly for CD8+ T cell tolerance, this APC must be capable of capturing exogenous antigens and cross-presenting them in class I pathway. Various cells types have been shown to have the capacity to cross present exogenous antigens in vitro, including myeloid-derived DCs, macrophages, and B cells.”

Unlike the factors regulating the balance between tolerance and ignorance, the factors determining the choice between tolerance and priming are not well understood. According to Heath, et al., what determines the choice between tolerance and priming “is probably one of the outstanding questions at the moment.” According to Sallusto and Lanzavechia (1999⁵⁴⁶) in another recent review: “finding the factors that regulate the balance between tolerance and response is now considered the holy grail of immunology.”

(ii) Two-Peaks

(a) O'Brien 1996

An intervention induces trigger apoptosis in insulin producing β cells. According to the two-peak model, if the trigger apoptosis is substantial, such intervention should produce two-peak apoptosis and substantial decrease in the number of viable β cells.

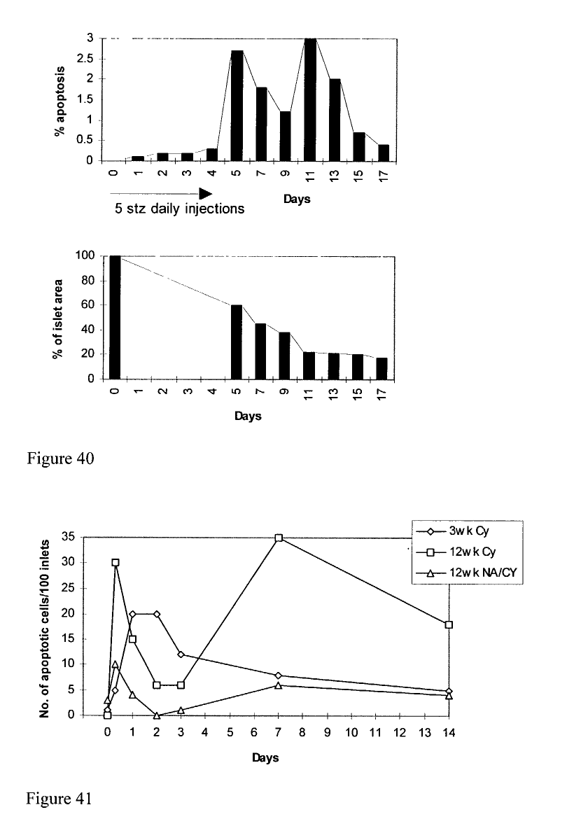

Five to six week old male C57B1/6 mice were injected low-dose (40 mg/kg body weight) streptozotocin (stz) per day for five consecutive days. Two-peaks in the incidence of β cell apoptosis occurred. The first peak at day 5, which corresponded to an increase in blood glucose concentration, and the second at day 11, when lymphocytic islet infiltration (insulitis) was maximal (O'Brien 1996⁵⁴⁷, FIGS. 3 and 4. See FIG. 40).

Insulitis did not begin until day 9, by which time treated animals had developed overt diabetes. β-cell apoptosis preceded the appearance of T-cells in the islets and continued throughout the period of insulitis. This study supports the two-peak model were the first peak is trigger apoptosis and the second is T-cell induced apoptosis.

(b) O'Brien 2000

An intervention increases oxidative stress in β cells and dendritic cells. Pancreatic islets are especially susceptible to oxidative stress. A study showed that low gene expression of the antioxidant enzymes superoxide dimutase (SOD), catalase, and glutathione peroxidase in pancreatic islets compared with various other mouse tissues (Lenzen 1996⁵⁴⁸). Moreover, induction of cellular stress by high glucose, high oxygen, and heat shock treatment did not affect antioxidant enzyme expression in rat pancreatic islets or in RINm5F insulin-producing cells (Tiedge 1997⁵⁴⁹). Based on these results Tiedge, et al., concluded that “insulin-producing cells cannot adapt the low antioxidant enzyme activity levels to typical situations of cellular stress by an upregulation of gene expression.” The oxidative stress inducing intervention should, therfore, result in trigger apoptosis. According to the two-peak model, if the trigger apoptosis is substantial, such intervention produces two-peak apoptosis and substantial decrease in the number of viable β cells.

In mice, the first 3 postnatal weeks are characterized by marked changes in the activities of enzymes that protect against oxidative stress (glutathione peroxidase/reductase, catalase and superoxide dismutase), relative to older mice (Herman 1990⁵⁵⁰). It should be noted that Herman, et al., measured the expression of these enzymes in liver, lung and kindney tissues. However, let assume that DC in 3 week old mice are also protected against oxidative stress, and that β cell show a much lower level of protection (reasonble assumption in light of Tiedge, 1997 above). In such a case, according to the two-peak model, oxidative stress in 3-week-old mice should induce trigger apoptosis with a smaller shift to the right of the second peak, relative to older mice. Moreover, if the trigger apoptosis is also smaller in 3-week mice relative to older mice, it is possible that the sum of β cell apoptosis will show a single peak.

Finally, older mice treated with antioxidant and then oxidant should show attenuated two-peaks.

Consdier the results in the following study. A study administered a single intraperitoneal injection of cyclophosphamide (CY, 150 mg/kg body weight) to 3 and 12 week old male non-obese diabetic (NOD/Lt) mice. The study also administered, to another group of 12 week old mice, a single intraperitoneal injection of nicotinamide (NA, 500 mg/kg body weight) followed 15 minutes later by a single CY injection. The effect of these treatments on β cell apoptosis is presented in FIG. 41 (O'Brien 2000⁵⁵¹, FIG. 3).

The total number of apoptotic β cells were observed within the islets of Langerhans in haematoxylin and eosin-stained sections of the pancreata in all three groups harvested from 8 h until 14 days following treatment. However, the shape of the three curves representing the sum of β cell apoptosis is different. The 3 week mice under CY treatment show a single peak, the 12 week mice under CY show a two-peak curve, and the 12 week mice under NA/CY show attenuated two-peaks.

Since CY injection induces oxidative stress and NA is an antioxidant, these results support the predictions of the two-peak model.

(c) Hotta 1998

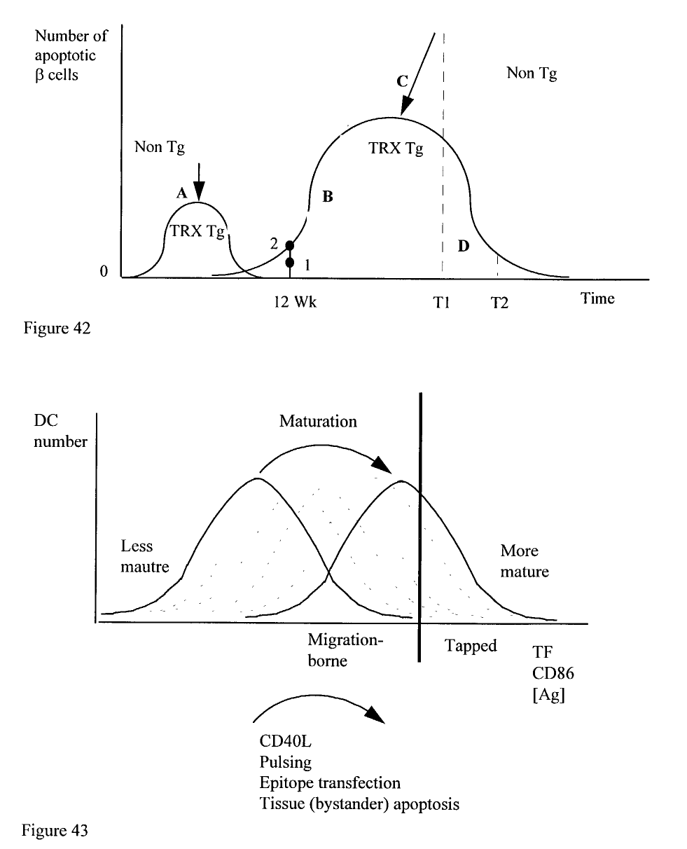

An intervention produced transgenic NOD mice (Tg) that overexpress thioredoxin (TRX), a redox-active protein, in β cells. The increased protection against oxidative stress reduces trigger apoptosis. According to the two-peak model, reduced trigger apoptosis, shifts the second peak left and down. Consider FIG. 42.

Morever, for simplicity, let assume that overt diabetes associates with destruction of a certain, fix number of β cells (in reality, it is actually a range and not fixed number). This number is reprsented by the sum of the areas (integrals) under the two-peaks. In the figure, the added area is restricted by dashed lines marked T1 and T2. Consider areas A, B, C and D. To represent the same number of apoptotic cells, A+C should be equal to B+D. A smaller area B results in larger area D, or delay in onset of diabetes. Let the distance between points 1 and 2 indicate the size of area B. A small distance indicates a small area B, and therefore, predicts a substaintial delay in onset of diabetes.

Consider the results of the following study. The average insulitis score of 12-wk-old female NOD transgenic mice and their female TRX negative littermates were 1.63±0.32 and 1.57±0.26 (mean SEM), respectively (Hotta 1998⁵⁵²). Although, the difference is statistically insignificant, the TRX Tg score is a little higher than the Non Tg score, as predicted by the model. Moreover, the small difference indicates a small area B, and therefore, a delay in onset of diabetes. As predicted, the first observed onset of diabetes was delayed from week 14 in Non Tg to week 23 in TRX Tg. Moreover, TRX Tg mice showed a markedly reduced cumulative incidence of diabetes at week 32 compared to Non Tg (Ibid, FIG. 4).

Similar observations are reported in Kubish 1997⁵⁵³

Numerous other studies showed reduced insulitis and delayed diabetes in NOD mice following treatment with antioxidants, such as, nicotinamide (vitamin B3) (Kim 1997⁵⁵⁴, Reddy 1990⁵⁵⁵), vitamin E (Beales 1994), lipoic acid (Faust 1994⁵⁵⁶), U78518F (Rabinovitch 1993⁵⁵⁷).

Cycolosporin reduces TF expression, therefore, reduces DC trapping and diabetes in NOD (Mori 1986⁵⁵⁸) and BB Wistar rats (Laupacis 1983⁵⁵⁹).

(iii) Autoimmune Disease

According to the “slow DC” model of autoimmune disease, an intervention that induces high expression of autoantigen on DC, and too little or too much expression of tissue factor (TF), produces tissue damage.

Presentation of high autoantigen concentration can result from transfection, immunization with autoantigen, increased apoptosis, etc. Insufficient TF surface concentration can result from, for instance, inhibition of TF transcription. Excessive TF expression can result from excessive antigen endocytosis (through oxidative stress), microcompetition, CD40L treatment, LPS treatment, etc. Consider the following studies.

(a) Studies with Lymphocytic Choriomeningitis Virus (LCMV)

(i) LCMV Characteristics

Let assume that LCMV is a GABP virus. This assumption is consistent with the following evidence. The glycoprotein (GP) protomer of the lymphocytic choriomeningitis virus (LCMV) has two N-boxes at positions (−44,−38) and (−3,+3). The distance between the two N-boxes is 35 bp. Of the dozens of known ETS factors, only GABP, as a tetrameric complex, binds two N-boxes. Typically, the N-boxes are separated by multiples of 0.5 helical turns (HT) (see discussion and references in the hormone sensitive lipase (HSL) gene above). There are 10 bp per HT. The 35 bp, or 3.5 helical turns separating the N-boxes in the GP promoter are consistent with characteristic GABP heterotetramer binding.

LCMV ARM 53b strain establishes a persistent infection in DC. Consider the following evidence. LCMV strains can be divided into two groups. The first group marked CTL-P+, includes viruses isolated from lymphocytes or macrophages obtained from CD4, perforin, and TNFα ko mice persistently infected for at least 7 months. These viruses failed to generate LCMV-specific CTL responses and caused a persistent infection. The second group marked CTL-P+, includes viruses isolated from CNS of TNFα ko mice. These viruses elicited a potent LCMV-specific CTL response, which cleared the virus within 2 wk and left no evidence of persistent infection. The Amstrong (ARM) 53b strain is a CTL-P+virus (Sevilla 2000⁵⁶⁰, Table I). According to Sevilla, et al., “first, DCs are the primary cell infected in vivo by CTL-P+LCMV variants; second, CTL-P+viruses astoundingly infect >50% of CD11c+(cellular marker for most DC in mouse lymphoid tissue) and DEC-205+ (antigen expressed on DC in lymphoid tissues) cells.”

Expression of a gene under the control of the rat insulin promoter (RIP) in transgenic mice induces a large number of immune susceptible cells. Consider the following evidence. Six percent transgenic mice, expressing the LCMV glycoprotein (GP), or nucleoprotein (NP), under control of the rat insulin promoter (RIP-GP, RIP-NP) in β cells, developed hyperglycemia. The pancreatic tissue of these mice revealed swollen islets with a group glass appearance (Oldstone 1991, FIG. 4A). No other treatment was neccessary to produce an immune reaction.

Other transgenic mice carring the hemagglutinin (HA) of the A/Japan/305/57 strain of influenza virus gene, or interferon-γ (IFNγ) under the control of RIP (RIP-HA and RIP-IFNγ, respectively), developed spontaneous diabetes with lymphocytic infiltration (Roman 1990⁵⁶¹, Sarvetnick 1990⁵⁶²). It is interesting that transgenic mice expressing IFNγ under control of rat glucagon promoter (RGP-IFNγ), which is expressed in a cells, did not develop diabetes. The increase in IFNγ concentration induced no net β cell destruction. The observed β cells apoptosis in transgenic RGP-IFNγ mice was compensated by vigorous regeneration. Specifically, the inlets showed no insulitis (Yamaoka 1999⁵⁶³). According to Yamaoka, et al., “IFNγ alone is insufficient for the complete destruction of β cells in vivo.” In terms of microcompetition, the microcompetition between the mouse's own insulin promoter (MIP) and the foreign rat's insulin promoter (RIP), reduces the expression of insulin, leading, eventually, to β cell destruction and trigger apoptosis. Therefore, RGP, which does not microcompete with MIP, does not produce diabetes.

(ii) Diabetes

RIP-GP transgenic mice show high GP expression in β cells (some mice spontaneously develop diabetes). However, most mice do not develop diabetes. In the resistant mice the expression of GP is not excessively high. In these mice, GP expression is not high enough to spontaneously produce autoimmune disease. According to the two-peak model, although antigen production is high (high trigger apoptosis), it is not sufficiently high to result in permanent β cell destruction and diabetes. Infection with LCMV excessively slows DC shifting the second peak right and up. This shift tips the balance in some resistant mice towards diabetes.

Consider the following studies.

1. Transgenic mice that express the viral glycoprotein (GP) or nucleoprotein (NP) from lymphocytic choriomeningitis virus (LCMV) under control of the rat insulin promoter (RIP-GP, RIP-NP) in pancreatic β cells develop autoimmune diabetes (IDDM) after infection with LCMV ARM 53b (Ohashi 1991⁵⁶⁴, Oldstone 1991⁵⁶⁵).

2. Adoptive transfer of autoreactive CD8+ cytotoxic T-lymphocytes (CTL) that are present in the periphery of RIP-GP or RIP-NP transgenic mice that were active in vitro and in vivo into uninfected transgenic recipients rarely resulted in hyperglycemia nor in insulitis, despite their ability to home to the islets and induce peri-insulitis (von Herrath 1997⁵⁶⁶). The weak trigger apoptosis induces peri-insulitis. However, without LCMV infection not enough DC are trapped in near the β cells to produce massive insulitis and significant T-cell induced apoptosis. In terms of the two-peak model, without LCMV infection, which slows DC, the second peak does not show enough shift to the right and up.

3. The P14 TCR single-transgenic model expresses a LCMV-GP specific T-cell receptor. In P14 transgenic mice tolerance is induced with repeated intravenous administration of the LCMV GP peptide epitope GP33. Peptide administration resulted in upregulation of T-cell activation markers, such as CD69 (Garza 2000⁵⁶⁷, FIG. 1a). In addition, whereas transgenic T-cells from untreated mice were incapable of lysing peptide pulsed target ex vivo, in vivo peptide treatment induced T-cell cytolytic activity (Ibid, FIG. 1b). Finally, peptide administration induced expansion of T-cells followed by deletions (Ibid, FIG. 1C).

Tissue circulating DC internalize the administered GP33 peptide. The DC moderately slow down, increase surface Ag expression and costimulation, and eventually migrate to the lymph node where they present the moderate concentration of surface Ag and costimulation to T-cells, causing activation, proliferation and deletion. Ex vivo treatment with GP33 fail to activate T-cell since activation requires presentation by DC.

Intravenous administration of GP33 to double transgenic mice (RIP-GP/P14) expressing GP on pancreatic β cells and LCMV-GP-specific T-cell receptor on T-cells surprisingly did not induce diabetes (Ibid, FIG. 2a).

In both models, administration of GP33 activates T-cells. However, since DC do not slow enough to be trapped in tissue, no homing signal is produced to chemoattract the activated T-cells to the inlets.

Immunization of the double transgenic mice intravenously with GP33 and FGK45, a rat anti-mouse-CD40 activating antibody, unlike immunization with GP33 and a rat polyclonal antiserum as iso-type-matched control, produced diabetes in all GP33+ banti-CD40 treated mice (Ibid, 2a). In both groups, the induction of T-cell activation markers and cytotoxic activity were identical. However, GP33+ control Ab produced mild pancreatic infiltration, while GP33+ anti-CD40 produced sever insulitis (Ibid, FIGS. 2b, c, d).

CD40 ligation on monocytes/macrophages induces TF cell surface expression. Specifically, treatment of purified monocytes with a with a stimulating anti-CD40 mAb (BL-C4) strongly induced monocyte procoagulant activity (PCA) which was related to TF expression as shown by flow cytometric analysis (Pradier 1996⁵⁶⁸). Exposure of monocytes/macrophages to cell membrane isolated from activated CD4+ T-cells (expressing CD40L), or a human rCD40L, increased TF surface expression and enzymatic activity (Mach 1997⁵⁶⁹, FIG. 2A, and B, Table). Anti-CD40L mAb blocked induction of TF in response to CD40 ligation. A similar effect on TF expression was observed in vascular smooth muscle cells (SMC) (Schonbeck 2000⁵⁷⁰).

CD40 ligation increases monocytes/macrophages and, most likely, dendritic cell, TF expression. TF expression on monocytes/macrophage and dendritic cells propels backward motility (see chapter on atherosclerosis above). A CD40L deficiency, therefore, should reduce dendritic cell migration to draining lymph node. A study analyzed the in vivo DC response to skin contact sensitization in CD40 ligand−/−mice. Immunohistochemistry of skin sections in unsensitized CD40 ligand−/−mice revealed no differences in terms of numbers and morphology of dendritic epidermal Langerhans cells (LC) compared to wild-type C57BL/6 mice. However, following hapten sensitization migration of LC out of skin was dramatically reduced and accumulation of DC in draining lymph nodes substantially diminished in CD40 ligand−/− mice compared to control (Moodycliffe 2000⁵⁷¹, FIGS. 2, 3). These observations are consistent with intact forward motility and deficient dendritic cell backward motility.

The effect of CD40 ligation on TF expression, can explain the results in Garza 2000 above. FGK45, the anti-CD40 agonist, increased TF expression on DC. The increased TF expression slowed down DC migration. As a result some DC arrived to the lymph node with increased surface GP33 concentration and costimulation. Other DC were trapped in the tissue. According to the slow DC model of autoimmune disease, the double transgenic mice treated with GP33 and FGK45 should develop diabetes. Moreover, Garza, et al., report that administration of GP33 and LPS, another inducer of TF expression, as expected, also resulted in diabetes.

(iii) Lupus

The H8 transgenic mice express the LCMV glycoprotein epitope (GP) 33-41 under control of a major histocompatibility complex (MHC) class I promoter. Since MHC class I is most likely expressed every cell, H8 mice express and present the GP33 epitope in every cell, specifically DC. Adoptive transfer of CD8+ T-cells from LCMV T-cell receptor transgenic mice into H8 mice led to efficient induction of peripheral tolerance after a period of transient activation and deletion (Ehl 1998⁵⁷²). In contrast, infection with LCMV, 1-3 days after T-cell adoptive transfer, resulted in disease. The mice showed signs of wasting 6-8 d after infection and 20-40% under specific pathogen-free conditions (up to 100% under non specific pathogen-free conditions) died within 12-15 d after infection. The remaining mice continued to lose weight and all died 3-5 mo after infection. Tissue examination revealed CD8+ T-cell infiltration in various organs, such as spleen, liver, gut, and skin (Ibid, FIG. 3). Infection of control mice did not lead to detectable clinical symptoms.

The spleen, liver, gut and skin show significant rate of tissue renewal indicating a considerable rate of normal cell apoptosis. This normal cell apoptosis loads antigens, including GP33, on surface of surveilling DC. DC internal expression of GP33 also loads antigens on these cells. However, the loadings produces GP33 (and other antigens) surface concentration only sufficient to generate tolerance and not T-cell infiltration. Infection of H8 mice with LCMV slows DC (some to a halt) in all tissues, resulting is increased antigen suface concentration. According to the tow peak model, the increase in antigen surface concentration and DC trapping result in T-cell infiltration in many tissues.

Compare RIP-GP and H8 transgenic mice infected with LCMV in terms of DC surface concentration the GP33 antigen.

| | RIP-GP | H8 |
|---|---|---|
| Pancreas | (Very) high tranfection | DC internal GP33 |
| | apop. + | expression + |
| | LCMV reduced backward | Low tissue renewal + |
| | motility | LCMV reduced backward |
| | | motility |
| Spleen | High tissue renewal + | DC internal GP33 |
| | LCMV reduced backward | expression + |
| | motility | High tissue renewal + |
| | | LCMV reduced backward |
| | | motility |
| Liver | High tissue renewal + | DC internal GP33 |
| | LCMV reduced backward | expression + |
| | motility | High tissue renewal + |
| | | LCMV reduced backward |
| | | motility |
| Gut | High tissue renewal + | DC internal GP33 |
| | LCMV reduced backward | expression + |
| | motility | High tissue renewal + |
| | | LCMV reduced backward |
| | | motility |
| Skin | High tissue renewal + | DC internal GP33 |
| | LCMV reduced backward | expression + |
| | motility | High tissue renewal + |
| | | LCMV reduced backward |
| | | motility |

In spleen, liver, gut and skin, internal expression of GP33 tips the balance from tolerance (or delayed infiltration) in RIP-GP mice, to T-cell infiltration in H8 mice (compare cells in table above for same tissue in both mice models). In pancreas, the lack of DC internal expression of GP33 in RIP-GP mice is probably more than compensated by the increase apoptosis in pancratic β cells induced by transfection with RIP-GP (see above).

The concepts presented in this table also predict that in H8 mice the rate of T-cell infiltration in different tissues is correlated with the rate of tissue renewal.

Another prediction suggested by this table is that any other treatment of H8 mice, which slows DC enough, produces similar results. Ehl, et al., tried a variety of infection and inflammtory stimuli. Specifically, they used 10 μg LPS. LPS increases TF expression on DC (see chapter on athersclerosis above) and therefore, slows DC backward motility. LPS treatement of H8 mice induced activation (Ibid, FIG. 8b).

Sytematic lupus erythematosus (also called disseminated lupus erythematosus, lupus, lupus erythematosus and SLE) is a chronic inflammatory autoimmune disease that affects many organs such as skin, joints, kidney, heart, lung and nervous system. At onset, only one organ system is usually invovled, however, additional organs may be affected later. A typical observation in lupus patients and animal models is spontaneous T-cell activation and organ infiltration.

Consider an infection with a GABP virus that result in sufficiently high viral genome number in circulating DC. Microcompetition between viral and TF N-boxes increases TF surface expression, which reduces DC backward motility. According to the two-peak model, the excessively slowing of DC backward motility induces pathologies similar to the symptoms observed in lupus patients. The organs affected first are those that show temporary or typical high trigger apoptosis (injured organs or organ with high tissue renewal).

Monocyte/macrophage infection with a GABP virus results in atherosclerosis (see chapter on atherosclerosis above). Both DC and macrophages originate from CD34+ progenitor cells (Hart 1997, ibid, FIG. 3). CD34+ cells are permissive for a GABP viral infection. For instance, Zhuravskaya, et al., (1997⁵⁷³) demonstrated that human cytomegalovirus (HCMV), a GABP virus, persisted in infected bone marrow (BM) CD34+ cells (see also, Maciejewski and St Jeor 1999 ibid, Sindre 1996, ibid). According to the proposed models, infection of CD34+ cells, therefore, result in both lupus and atherosclerosis. The observed concurrence of lupus and atherosclerosis is well documented. See for instance some recent reviews on the issue of accelerated atherosclerosis in systemic lupus erythematosus (Ilowite 2000⁵⁷⁴, Urowitz 2000⁵⁷⁵). Such observation are consistent with microcompetition, TF propelled backward motility, and the two-peak model.

Another interesting observation explained by these models is hypercoagulation thrombosis in lupus. The infection of CD34+ with a GABP virus increases TF expression on circulating monocytes. Such excessive TF expression in lupus was documented in a few studies (see, for instance, Dobado-Berrios 1999⁵⁷⁶). The excessive TF expression increases the probability of coagulation. (More on thrombosis in lupus and other diseases see the chapter on stroke.

(iv) Graft Versus Host Disease (GVHD)

DC from H8 mice (H8-DC) constitutively express the GP33 epitope. A single injection of 106H8-DC (high dose) to RIP-GP transgenic mice resulted in no glycemic change or transient increase in blood glucose to intermediate levels (15-20 mM), eventually returning to normal levels within a few days (Ludewig 1998⁵⁷⁷, FIG. 1A). A single injection of 10⁵H8-DC (intermediate dose) did not result in diabetes. However, repetitive H8-DC injections of intermediate doses, i.e., three doses of 105 DC in 6-d intervals (Ibid, FIG. 1C), or four doses of 104 DC in 2-d intervals (Ibid, FIG. 1D), resulted in T-cell infiltration (Ibid, FIG. 3) and diabetes. 50% of the repetitively immunized mice developed diabetes between day 10 and 14, while 40% developed hyperglycemia by days 18-21. Based on these observations Ludewig, et al., concluded that “the duration of antigenic stimulation by professional APCs, i.e., the integral of CTL activity over time, determines the disease outcome in this model of autoimmune diabetes.”

Consider a DC migrating “near” pancreatic β cells at a certain speed. During the time the DC spends “near” the β cells, it has a certain probability, denoted P, to internalize a certain concentration [Ag] of β cell antigens. Now, consider two DC also migrating at this speed. Assuming independent DC migration and internalization, the probability that at least one of them internalizes [Ag] is 2P (the independent assumption does not hold if, for instance, the two DC co-migrate and end up internalizing a portion of [Ag] each). Under the independent assumption, an increase in the number of migrating DC, without change in other conditions, increases the probability of antigen internalization. Consider, as an alternative situation, one DC migrating at half the original speed. Since the time the DC spends near the β cells is twice as long, its probability that the cell internalize [Ag] is 2P, the same as the probability of the two DC migrating at the original speed. Increasing the number of migrating DC and slowing migration of the existing pool of DC produce the same effect. Repetitive immunization with H8-DC is equivalent to slowing DC backward motility. Since the integral of T-cell induced apoptosis over time determines the outcome of autoimmune disease in the case of slow DC migration (see two-peak model above), the same integral is important in the case of repetitive DC immunization.

Graft-versus-host disease (GVHD) is a complication following allogeneic bone marrow (BM) transplantation (BMT). A typical observation in GVHD patients is spontaneous T-cell activation and organ infiltration. Approximately, 50% of patients undergoing allogeneic BMT with related HLA-matched donor develop GVHD.

A study measured the percentage of DC present in blood mononuclear cells (MNC) in patients following allogeneic and autologous stem cell transplantation and healthy controls. The mean number of DC as a percentage of MNC was 0.58%, 0.40% and 0.42%, for patients following allogeneic transplantation showing no GVH symptoms, patients following autologous transplantation, and healthy controls, respectively (P=0.06 for the difference between allogeneic and autologous patients) (Feamley 1999⁵⁷⁸, FIGS. 3, 6). These results indicate that allogeneic stem cell transplantation increases DC number. The higher DC number increases the probability of antigen internalization. In tissues with high normal apoptosis (rapidly renewing tissues), such an increase might result in T-cell infiltration and tissue apoptosis.

(v) Vaccination with DC

Let the expression of TF, CD86 and level of antigen presentation on DC (denoted [Ag]) be correlated. Treatment with CD40L, pulsing, apoptosis of tissue of bystander cells, transfection with a gene expressing an epitope increase TF, CD86 and [Ag]. This increase is called maturation. Let assume that the distribution of number of DC expressing TF, CD86 and [Ag] is normal. Consider FIG. 43.

Maturation in the figure is represented by a shift of the DC distribution to higher TF, CD86 and [Ag] values. According to the TF propelled backward motility model there exists a certain level of TF expression that traps DC. This level is marked with a thick line in the figure. A cell with lower TF concentration is migration-borne (capable of migrating). A cell with higher TF concentration is traped.

Consider vaccination with two kinds of cells, less mature and more mature, denoted with solid lines in the figure. This model provides the following predictions. Vaccination with the less mature cells induces no trapping. All cells migrate out of tissue. In contrast, vaccination with more mature cells induces cell trapping. Some cells migrate out of tissue, represented by the area under the DC distribution left of the thick line), while the rest are trapped (the area right of the thick line).

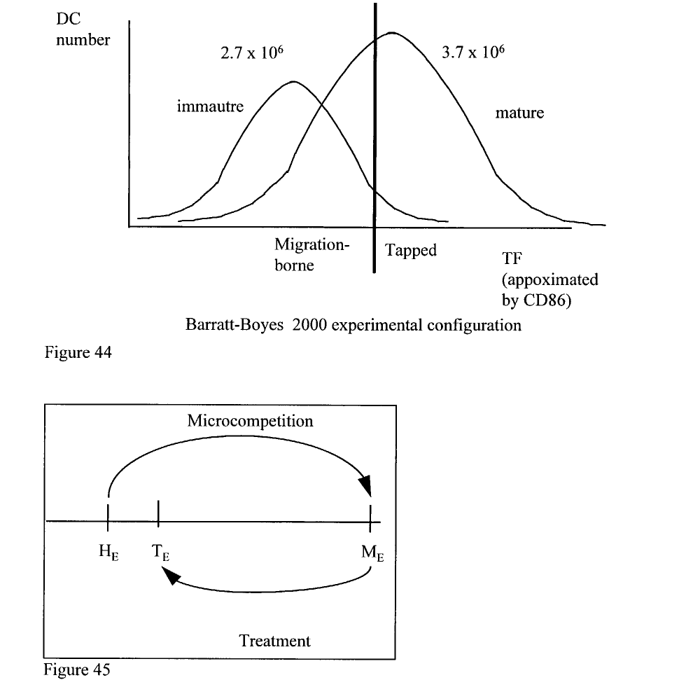

Consider the following study. DC from CD14+ peripheral blood monocytes of rhesus macaques were cultured for 4 days in GM-CSF and IL-4. The cells show no expression of CD83, the mature DC marker, moderate expression of the costimulatory molecules CD80, CD86, and CD40, and high levels of MHC class I and class II (Barratt-Boyes 2000⁵⁷⁹, FIG. 1). These cells were designated immature DC. Other cells were cultured for additional 2 days (total of 7 days) with added CD40L, a known inducer of rapid maturation. The addition of CD40L induced uniform expression of CD83, and high expression of CD80, CD86, and CD40 (Ibid, FIG. 1). These cells were designated mature DC. To determine the relative efficiency of immature and mature DC migration, the site of injection was inspected 36 h after injection of cells. Injection of 2.7×10⁶ immature DC resulted in minor localized acute inflammatory response. No fluorescently labeled cells could be identified at that time. In contrast, injection of 3.7×10⁶ mature DC resulted in a severe acute inflammatory infiltrate at the site of injection in two out of three animals. A large number of fluorescently labeled DC was detected in the dermis at 35 h in these animals (Ibid, FIG. 8). The experimental configuration in this study is presented in FIG. 44.

Many more mature DC are trapped following injection with mature rather than immature cells. Compare the areas right of the thick line under the mature and immature curves. According to the study the size of the area representing the trapped DC following injection of immature cells should be zero. However, according to the two-peak model, to produce T-cell infiltration, some DC should be trapped. This inconsistency can be resolved if we assume that the infiltration T-cells cleared most of the few trapped cells before the 36 hours inspection.

This study reports another important observation. Following injection of immature and mature DC, a portion of the injected cells (0.07-0.12%) reached the lymph node (Ibid, FIG. 7) producing an immune reaction at the injection site. In terms of the figure above, in both cases the area under the curves, left of the thick line, is not empty. Both injections included migration-borne DC. Similar observations are reported in Hermans 2000⁵⁸⁰. However, not all injected DC migrate to the lymph node. Some enter circulation. These DC can end up in any tissue. According to the discussion above, if enough injected DC enter circulation over an extended period of time, they might produce an immune reaction in tissues with abnormally high epitope expression or rapidly renewing tissues. Consider the following studies.

SM-LacZ transgenic mice widely express the β-galactosidase (β-gal) antigen in cardiomyocytes of the right ventricle and in arterial smooth muscle cells. Repetitive treatment of SM-LacZ mice with DC presenting β-gal peptide resulted in vascular immunopathology with strong lymphocytic infiltration in small and medium-sized arteries and in the right ventricle (Ludewig 2000⁵⁸¹). Immunization of SM-LacZ mice with DC pulsed with an irrelevant peptide produced a mild liver infiltration and no anti-β-gal CTL activity. Immunization of nontransgenic mice with DC presenting the β-gal peptide also produced a mild liver infiltration and no anti-β-gal CTL activity. Naive SM-LacZ mice showed no specific CTL reactivity (Ibid, FIG. 2B). Similar observations of autoimmune disease induce by DC immunization is reported in Roskrow (1999⁵⁸²).

(b) Studies with Theiler's Murine Encephalomyelitis Virus (TMEV)

(i) TMEV Characteristics

TME viruses are members of the genus Cardiovirus in the family Picornaviridae. These viruses can be divided into two groups based on their neurovirulence characteristics following intracerebral (i.e.) inoculation of mice. Highly virulent strains, such as GDVII virus, cause rapidly fatal encephalitis. The less virulent strains, such as BeAn and DA show at least a 10-fold reduction in the mean 50% lethal dose (LD₅₀) compared to the virulent strains. Moreover, they can establish a persistent infection in the central nervous system (CNS).

Let assume that all three TMEV strains, GDVII, BeAn and DA are GABP viruses. This assumption is consistent with the following evidence. The 5′ UTR of all three strains includes 9 N-boxes. Moreover, the 5′ UTR of all three strains includes a pair of N-boxes (positions (−129,−123) and (121,−115), or positions (935,941) and (943,949) when numbered according to the BeAn sequence). It is interesting that the pair in GDVII is different than the pair in BeAn and DA. In GDVII the pair of N-boxes (underlined) is CTTCCGCTCGGAAG while the pair in BeAn and DA is CTTCCTCTCGGAAG. The GDVII pair is symmetrical while the pair in BeAn and DA is not. The asymmetry in BeAn and DA might result in reduced affinity to GABP, and therefore, reduced rate of transcription initiation. This interpretation is consistent with the following evidence.

In a series of experiments, Lipton and co-workers attempted to identify the DNA sequence responsible to the difference in these strains virulence. In these studies they constructed recombinant TMEVs by exchanging corresponding genomic regions between GDVII and BeAn. One such recombinant virus is Chi 5L, in which the (933,1142) BeAn sequence replaces the original GDVII sequence. Inoculation of Chi 5L into mice by the i.e. route showed attenuated neurovirulence. The LD₅₀ value for Chi 5L was ≧7.5×10⁵ in comparison to 10 for GDVII (Lipton 1998⁵⁸³, table 1). Replacing the original GDVII pair of N-boxes with the BeAn pair resulted in reduced virulence.

(ii) Demyelination (Multiple Sclerosis)

As with many other viruses, TMEV infection spreads from cell to cell. However, the identity of infected cells and order of viral cell-to-cell spread determines the clinical outcome. Consider an infection with a BeAn and DA virus. The firsT-cells infected in the nervous system are neurons. The infection results in cell apoptosis. The cell debris is internalized by surveilling macrophages, slowing the cells backward motilitiy, trapping a few cells which induces T-cell infiltration. These events are characteristic of the acute phase, which terminates when the neuronal infection is cleared, inflammation in gray matter subsides, and neuron apoptosis returns to normal levels. However, during the acute phase the virus spreads from neurons to some infiltrating macrophages, establishing a persistent infection. The infection increases surface TF expression, slows backward motility of some macrophages and traps others in the white matter. Since infection is not lytic, trapped macrophage continue to internalize schwann cell/olgiodendrocyte debris or apoptotic cells produced in normal cell turnover, or as a result of myelin damage. The internalized myelin is processed and presented on cell surface. The loaded macrophage releases cytokines providing a homing signal to T-cells and new infiltrating macrophages. Both trapped macrophages and Schwann cells/oligodendrocytes present myelin on their surface bound to MHC. The infiltrating T-cells bind the presented myelin on trapped macrophage and Schwann cells/oligodendrocytes and destroy them. The result such destruction is demyelination. The observations in the following studies support such a sequence of events.

Tsunoda, et al., (1997⁵⁸⁴) show that the firsT-cells infected in the nervous system are neurons and that the initial limited inflammation in the gray matter subsides concurrently with the decline in neuronal apoptosis. Similar observations are reported by Ha-Lee, et al., (1995⁵⁸⁵).

According to Lipton, et al., (1995⁵⁸⁶) virus antigen(s) was first detected in the white matter on day 14 post inoculation. On days 14 and 22, virus antigen(s) was occasionally seen within long stretches of axons extending from the gray matter into anterior white matter (Ibid, FIG. 2A). MOMA-2-positive cells (MOMA-2 is a monoclonal antibody to macrophages), some of which contained virus antigen(s), were observed in close proximity to infected axons (Ibid, FIG. 2A). This observation suggests that TMEV leaves the gray matter by axonal spread, is released from the axoplasm as motor neurons, and then secondarily infects macrophages in the white matter. The fact that motor neurons are the principle virus target in the acute gray matter phase of infection and the predominantly anterolateral location of virus antigen-positive cells in the white mater on days 14, 22, and 29 support this conclusion. Increasing umber of virus antigen-positive, MOMA-2-positive cells appeared in the thoracic cord white matter between days 14 and 49 and then remained at this level of infection until day 73. However, only a small fraction of MOMA-2-positive cells contained virus antigen(s) during this period (Ibid, FIG. 2B). The early infiltration and apparent spread of these cells from anterior to posterior in the spinal cord, with a tendency for virus antigen-positive cells to be found at the periphery of advancing edges of lesions (Ibid, FIG. 3), also supports this conclusion. Based on these observation Lipton, et al., concluded that at least some of the MOMA-2-positive cells have a hematogenous origin, and that infection occurs upon entry of these cells into the CNS.

Miller, et al., (1997⁵⁸⁷) reports the temporal appearance of T-cell response to viral and known encephalitogenic myelin epitopes in TMEV-infected SJL/J mice. Clinical signs being approximately 30 days after infection and display chronic progression with 100% of the animals affected by 40-50 days postinfection. Ultraviolet light (UV)-inactivated TMEV produced a T-cell proliferation in spleen of infected mice both at day 33 postinfection, concomitant with onset of clinical signs, and at day 87. In contrast, at 33 postinfection, the major encephalitogenic epitope on myelin proteolipid protein (PLP139-151 and PLP178-191) and myelin basic protein (MBP84-104) did not produce T-cell proliferation in spleen, cervical or pooled peripheral lymph nodes. However, a response to PLP139-151 was observed in all lymphoid compartments at day 87 postinfection. Similar temporal observations are associated with the appearance of CD4+ Th1-mediated delayed-type hypersensitivity (DTH) responses. The immunodominant TMEV VP270-86 epitope produced DTH at all times tested. In contrast, the PLP139-151 epitope first produced DTH only at day 52, persisting through day 81 postinfection (Ibid, FIG. 1C). Assessment of DTH to a larger panel of encephalitogenic myelin epitope during late chronic disease (164 days postinfection), showed persistence of peripheral T-cell reactivity to both VP270-86 and PLP178-151 and appearance of responses to multiple, less immunodominant myelin epitopes including PLP56-70, PLP178-191, and the immunodominant myelin oligodendrocyte glycoprotein epitope (MGO92-106) (Ibid, FIG. 1d). The study calls these observations “epitope spreading” and defines it as the process whereby epitopes distinct from and non-cross-reactive with an inducing epitope become major targets of an ongoing immune response. The longer macrophages are trapped in white matter, the higher the concentration of presented epitopes on cell surface. Since “rare” epitopes require longer macrophage residence time to accumulate at high enough concentrations, the reported epitope spreading indicates abnormally long macrophage residence time, or abnormally high macrophage trapping.

(b) Human Studies

Numerous studies report similar observations in all autoimmune diseases. Consider T-cell infiltration as an example. For the sake of brevity, in every disease we report observations that relate to different aspects of the above models.

(i) Diabetes

1. According to the “excessively slow” DC model, tissue cell destruction follows T-cell infiltration. T-cell infiltration, or insulitis, was extensively reported in pre-diabetic and recent-onset diabetic patients, see, for instance, Signore (1999⁵⁸⁸, a review), Foulis (1991⁵⁸⁹), Foulis (1984⁵⁹⁰).

2. Coxsackie B4 virus infect pancreatic β-cells inducing limited β cell death (Roivainen 2000⁵⁹¹). The limited β-cell destruction does not result in diabetes. However, according to the two-peak model, the “trigger apoptosis” result in T-cell infiltration. According to the excessively slow DC model, in individual harboring a GABP virus the T-cell induced apoptosis might result in diabetes. Consistent with this prediction, some recent studies found a strong association between Coxsackie B4 virus infection and onset of insulin-dependent diabetes mellitus in humans (Andreoletti 1998⁵⁹², Anderoletti 1997⁵⁹³, Frisk 1997⁵⁹⁴, Clements 1995⁵⁹⁵). If Coxsackie B4 is a GABP virus, and can infect DC, the cellular events resulting from a Coxsackie B4 viral infection resemble the events of a TMEV infection (see above).

(ii) Multiple Sclerosis (MS)

1. According to the “excessively slow” DC model trapped DC show high expression of B7, specifically B7.2 (also called CD86). Therefore, plaque from MS patients, and specifically trapped macrophages, should show high expression of B7. Consider the following studies.

Infiltrating macrophages in brain sections from MS patients showed significant B7 immunoreactivity, in contrast to normal brains, which showed no B7 immunoreactivity (De Simone 1995⁵⁹⁶). Another study found B7.1 staining in plaque from MS patients localized predominantly to lymphocytes in perivenular inflammatory cuffs, and B7-2 staining predominantly on macrophages in inflammatory infarcts (Windhagen 1995⁵⁹⁷).

2. According to the “excessively slow” DC model trapped DC express chemokine, such as MIP-1α, MIP-1β and RANTES. Therefore, plaque from MS patients, and specifically trapped macrophage, should show high expression of these chemokines. Consider the following studies.

A study measured expression of the CC chemokines MIP-1α, MIP-1β, and RANTES in brain tissue from MS patients using reverse transcriptase-polymerase chain reaction techniques. Both MIP-1β and RANTES were significantly elevated in brain tissue of MS patients. In addition, MIP-1α was also increased, although not significantly. Immunohistochemistry revealed that MIP-1α and MIP-1β immunoreactivity was predominantly found in perivascular and parenchymal macrophages containing myelin degradation products (Boven 2000⁵⁹⁸).

(iii) Psoriasis (Ps) and Atopic Dermatitis (AD)

1. The effectiveness of the immune system deteriorates with age (see reviews Khanna 1999⁵⁹⁹, Ginaldi 1999⁶⁰⁰), which might explain the increased incidence of infectious diseases in the aged. Consider an individual harboring a persistent infection of a GABP virus in DC (for instance, cytomegalovirus). At every age, the balance between two forces, the virus drive to replicate, and the capacity of the immune system to control or clear the infection, determines the viral genome copy number present in infected cells. A decline in immune system effectiveness, therefore, increases viral genome copy number. Consistent with that conclusion, Liedtke, et al., (1993⁶⁰¹) showed an increase in the prevalence of herpes simplex virus 1 (HSV-1) neuronal latency with age.

Increase in viral genome copy number intensifies microcompetition, which slows DC and result in higher [Ag] and [B7] on surface of DC reaching the draining lymph node. The increase in [Ag] and [B7] increases [DC·T], which increases the probability of Th1 vs. Th2 differentiation. This argument predicts a decline in Th2 and increase in Th1 autoimmune diseases with age. Consider the following evidence.

Atopic dermatitis (AD), is a Th2 disease, while psoriasis (Ps) is a Th1 disease. A study systematically examined patients attending a dermatology clinic for the presence of AD and/or Ps. Nine hundred and eighty-three patients were studied—224 with AD, 428 with Ps, 45 with both AD and Ps, and 286 controls. The results showed that 16.7% of the AD patients had also Ps, and 9.5% of Ps patients had AD. In consecutive occurrences, Ps generally followed AD (Beer 1992⁶⁰²). Out of the 45 patients with both AD and Ps, 26 patients had onset of AD first and Ps later in life (average age=10 and 26, respectively), 9 subjects (all children) had simultaneous onset of AD and Ps, and 1 patient had first onset of Ps at the age of 16, followed by AD+Ps at the age of 18 and return to Ps.

2. Increase in CTLA4Ig decreases [DC·T] (see formula above). As a result, T-cell induced apoptosis decreases, which decreases inflammation (DC infiltration, T-cell infiltration, etc). Consider the following studies.

Patients with psoriasis vulgaris received four intravenous infusions of the soluble chimeric protein CTLA4Ig (BMS-188667) in a 26-wk, phase I, open label dose escalation study. Clinical improvement was associated with reduced cellular activation of lesional T-cells and DC. Concurrent reductions in B7.1 (CD80), B7.2 (CD86) were detected on lesional DC, which also decreased in number within lesional biopsies. Skin explant experiments suggested that these alterations in activated or mature DCs were not the result of direct toxicity of CTLA4Ig for DC (Abrams 2000⁶⁰³). Based on these observations, Abrams, et al., concluded that “this study highlights the critical and proximal role of T-cell activation through the B7-CD28/CD152 costimulatory pathway in maintaining the pathology of psoriasis, including the newly recognized accumulation of mature DCs in the epidermis.”

3. According to the “excessively slow” DC model trapped DC show high expression of B7, specifically B7.2 (also called CD86). Therefore, lesions from AD and Ps patients, and specifically trapped DC, should show high expression of B7. Moreover, since DC increase B7 expression while migrating out of tissue, in case of Langerhans cells, while migrating from epidermis to dermis and than lymph vessel, B7 expression on Langerhans cells in dermis should be higher than cells in epidermis. Consider the following studies.

A study measured the expression of co-stimulatory molecules in AD and Ps patients. B7.2 and B7.1 were detected on dendritic-shaped cells not only in the epidermis but also in the dermis in the inflammatory lesions of atopic dermatitis (n=12). B7.2 was expressed in all cases (100%), while B7.1 was expressed in only five cases (42%). These molecules were not detected in normal control subjects (n=8) (Ohki 1997⁶⁴). Neither B7.1 nor B7.2 was detected on keratinocytes. Stronger expression of B7.2 over B7.1 was also observed in psoriasis vulgaris (n=11). The expression rate of these molecules on Langerhans cells increased in the dermis.

4. A persistent infection of DC with a GABP virus increases the probability of developing an autoimmune disease. Moreover, an increase in viral load should exacerbate the disease. Consider the following studies.

To detect active infection a study compared the antigen expression of cytomegalovirus (CMV), a GABP virus, in peripheral blood mononuclear cells (PBMC) from psoriatic patients (n=30) with healthy volunteers (n=65). The results showed higher CMV antigenaemia in psoriasis (43%) compared with healthy laboratory staff (12%, P<0.01) and blood donors (6%, P<0.001) (Asadullah 1999⁶⁰⁵).

Another study reports the development of psoriasis vulgaris in Four patients suffering from immune deficiency related to HTLV III, a GABP virus. The psoriasis was extensive, exsudative, and almost refractory to therapeutical approaches. The bulk of dermal infiltrating mononuclear cells were CD8+ T lymphocytes (Steigleder 1986⁶⁰⁶).

HIV is a GABP virus. According to a recent review (Mallon 2000), “psoriasis occurs with at least undiminished frequency in HIV-infected individuals.” Moreover, according to the paper, “It is paradoxical that, while drugs that target T lymphocytes are effective in psoriasis, the condition should be exacerbated by HIV infection.” See also the review by Montazeri, et al., (1996⁶⁰⁷). Another study reported clinical improvement of HIV-associated psoriasis in parallel with a reduction in HIV viral load induced by effective antiretroviral therapy (Fischer 1999⁶⁰⁸).

(4) Other Autoimmune Diseases

There many more autoimmune diseases not discussed above. Some are asthma, rheumatoid arthritis and thyroiditis. As predicted by the excessively slow DC and the two-peak models, studies with patients and animal models of these diseases report observations similar to the ones already mentioned. For instance, studies in animal models of asthma showed that DC collect antigens in the airways, upregulate [Ag] and [B7], migrate to the thoracic lymph nodes where they present the antigens to T cells (Vermaelen 2000⁶⁰⁹). Other studies showed that DC are essential for development of chronic eosinophilic airway inflammation in response to inhaled antigen in sensitized mice (Lambrecht 2000A 610, Lambrecht 2000B⁶¹¹, Lambrecht 1998⁶¹²). More studies showed the significant role of B7 in allergic asthma (Mathur 1999⁶¹³, Haczku 1999⁶¹⁴, Padrid 1998⁶¹⁵, Keane-Myers 1998⁶¹⁶). Similar observations were reported in rheumatoid arthritis (see, for instance, Balsa 1996⁶¹⁷, Liu 1996⁶¹⁸), and thyroditis (see, for instance, Wantanabe 1999⁶¹⁹, Tandon 1994⁶²⁰).

6. Discovery 6: Other Disruptions of GABP Pathway

(1) Drug Induced Molecular Disruptions

Microcompetition disrupts the GABP pathway. Some drugs also disrupt this pathway. As a result these drugs induce “side effects” similar to the clinical symptoms characteristic of microcompetition. Some of these side effects are weight gain, insulin resistance, and hypertension. The following sections propose the mechanism underlying these side effects.

(a) Cytochrome P450

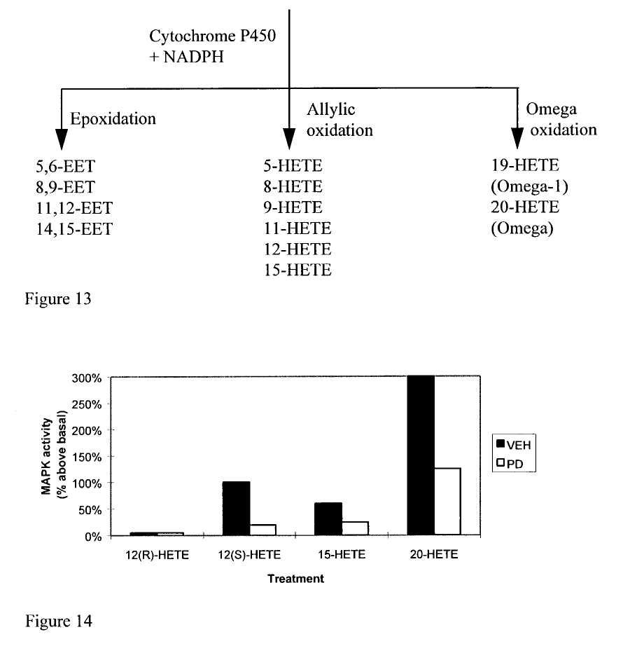

Three distinct pathways of arachidonic acid (AA) oxidation have been described. The enzyme systems involved are regiospecific and stereospecific. Of the three pathways, the products of the cyclooxygenase and lipoxygenase pathways have been extensively researched. Research on the products of the “third pathway”, the cytochrome P450-dependent monooxygenases, is less extensive. The “third pathway”, mediated by CYP enzymes, uses NADPH and molecular oxygen in a 1:1 stoichiometry. Three types of oxidative reactions are known to occur. Olefin epoxidation (epoxgenases) produces 4 sets of regio-isomers, the epoxyeicosatrienoic acids (EETS), specifically, the (5,6-), (8,9-), (11,12-) and 14,15-EETs. Allylic oxidation produces hydroxyeicosatetraenoic acids (HETEs), specifically, (5-), (8-), (9-), (11-), (12-) and 15-HETEs. Omega oxidation produces the 19- and 20-HETEs. These sets are sumerized in FIG. 13.

(b) Arachidonic Acid Metabolites Activate ERK

Rabbit VSMCs were treated with the vehicle dimethyl sulfoxide (DMSO) alone or 20 [M PD98059 (PD) for 4 h and then exposed to 0.25 μM 12(R)-, 12(S)-, 15, or 20-hydroxyeicosatetraenoic acid (HETE) for 10 min. FIG. 14 presents MAP kinase activity in these cells (Muthalif 1998⁶²¹, FIG. 3A).

The study also showed that 20-HETE specifically activated ERK1 and ERK2 (Ibid, FIG. 3D). Similar activation of MAPK by 12-, and 15-HETE are reported in Wen 1996⁶²² and Rao 1994⁶²³. Another study tested the effect of 14,15-epoxyeicosatrienoic acid (EET) on ERK activation. LLCPKc14, an established proximal tubule epithelial cell line derived from pig kidney, were treated with 14,15-EET (20 μm) for 15 min, then tyrosine phosphorylated proteins in cell lysates were immunoprecipitated with anti-phosphotyrosine antibodies and immunoblots probed with an antibody which recognizes ERK1 and ERK2. The results showed that 14,15-EET stimulated ERK1 and ERK2 phosphorylation (Chen 1999¹²⁴, FIG. 2D).

To summerize, 12(S)-, 15, or 20-HETE and 14,15-EET activate ERK. In other words, these arachidonic acid metabolites are ERK agents.

(c) 12(S)-, 15, or 20-HETE and 14,15-EET CYP Specific Enzymes

The following table lists a few cytochrome P450 enzymes that produce ERK agents metabolites. We call these enzymes CYP-ERKs. When the study is tissue specific, the tissue type is mentioned in the reference column.

| Enzyme | ERK agent product | Reference* |
|---|---|---|
| CYP1A2 | 14, 15-EET | Ritkind 1995 (human liver) |
| CYP2B4 | 14(R), 15(S)-EET | Zeldin 1995 (lung) |
| CYP2C8 | 14, 15-EET | Rifkind 1995 (human liver) |
| CYP2C9 | 15(R)-HETE | Bylund 1998, |
| | 12-HETE | Rifkind 1995 (human liver) |
| CYP2C19 | 14, 15-EET | Bylund 1998, Keeney 1998 |
| | | (14S 15R, skin keratinocytes) |
| | 12R-HETE | Keeney 1998 (skin keratinocytes) |
| | 15R-HETE | Keeney 1998 (skin keratinocytes) |
| CYP2C23 | 14, 15-EET | Imaoka 1993 (rat kidney) |
| CYP2C29 | 14, 15-EET | Luo 1998 |
| CYP2C39 | 14, 15-EET | Luo 1998 |
| CYP2C37 | 12-HETE | Luo 1998 |

*Bylund 1998⁶²⁵, Imaoka 1993⁶²⁶, Zeldin 1995⁶²⁷, Rifkind 1995⁶²⁸, Luo 1998⁶²⁹, Keeney 1998⁶³⁰

(d) Drug Inhibition of CYP-ERK and Microcompetition-Like Diseases

Microcompetition reduces the expression of GABP stimulated genes and increases the expression of GABP suppressed genes. Inhibition of an ERK agent produces the same effect. Consider a drug that only inhibits CYP-ERK. That is, the drug has no other chemical reactions, such as inhibition of another enzyme. Call such a drug an “empty” drug. An empty drug should produce the same clinical profile as microcompetition.

The following table lists drugs, which inhibit CYP-ERKs and their microcompetition-like side effects (mostly weight gain, some insulin resistance and atherosclerosis).

| | | |
|---|---|---|
| | | Microcompetition- |
| Drug | Cytochrome P450 (CYP type) | like symptoms |

Cytochrome P450 inhibitors

| | | |
|---|---|---|
| Phenytoin | Kidd 1999⁶³¹ (CYP2C9) | Egger 1981⁶³⁴ |
| | Ring 1996⁶³² (CYP2C9) | |
| | Miners 1998⁶³³ (CYP2C9) | |
| Glipizide | Kidd 1999 (ibid) (CYP2C9) | Campbell 1994⁶³⁵ |
| Carbamazepin | Petersen 1995⁶³⁶ (CYP2C9) | Hogan 2000⁶³⁸ |
| | Meyer 1996⁶³⁷ | Mattson 1992⁶³⁹ |
| | (through drug interaction) | |
| Valproic | Sadeque 1997⁶⁴⁰ (check) | Bruni 1979⁶⁴¹ |
| Acid, sodium | (CYP2C9) | Egger 1981 (ibid) |
| valproate | | Zaccara 1987⁶⁴² |
| | | Mattson 1992 (ibid) |
| | | Sharpe 1995⁶⁴³ |
| Losartan | Song 2000⁶⁴⁴ (CYP2C9) | Camargo 1991⁶⁴⁶ |
| | Meadowcroft 1999⁶⁴⁵ (CYP2C9) | |
| | Miners 1998 (ibid) (CYP2C9) | |
| Simvastatin | Transon 1996⁶⁴⁷ (CYP2C9) | Matthews 1993^{648,I} |
| Olanzapine | Ring 1996 (ibid) (CYP2C9) | Osser 1999⁶⁴⁹ |
| | | Koran 2000⁶⁵⁰ |
| Clozapine | Ring 1996 (ibid) (CYP2C9) | Osser 1999 (ibid) |
| | Fang 1998⁶⁵¹ (CYP2C9) | |
| | Prior 1999⁶⁵² (CYP1A2, | |
| | CYP2C19) | |
| Fluvoxamine | Olesen 2000⁶⁵³ (CYP1A2, | Harvey 2000^{655,II} |
| Fluoxetine | CYP2C19) | Sansone 2000⁶⁵⁶ |
| (Prozac) | Miners 1998 (ibid) (CYP2C9) | Michelson 1999^{657,II} |
| | Schmider 1997^{654 (CYP2C9)} | Darga 1991^{658,II} |
| Tolbutamide | Ring 1996 (ibid) (CYP2C9) | Wissler 1975^{660,III} |
| | Miners 1998 (ibid) (CYP2C9) | Ballagi-Pordany |
| | Lasker 1998⁶⁵⁹ (CYP2C9, | 1991^{661,III} |
| | CYP2C19) | |
| Anastrozole | Grimm 1997⁶⁶² (CYP1A2, | Wiseman 1998⁶⁶³ |
| | CYP2C9) | Lonning 1998⁶⁶⁴ |
| | | Buzdar 1998⁶⁶⁵ |
| | | Jonat 1997⁶⁶⁶ |
| | | Buzdar 1997⁶⁶⁷ |
| | | Hannaford 1997⁶⁶⁸ |
| | | Buzdar 1997⁶⁶⁹ |
| | | Buzdar 1996⁶⁷⁰ |
| | | Jonat 1996⁶⁷¹ |
| Nelfinavir (PI) | Khaliq 2000⁶⁷² (CYP2C19) | VI |
| | Lillibridge 1998⁶⁷³ (CYP2C19, | |
| | CYP1A2)^{,V} | |
| Ritonavir (PI) | Muirhead 2000⁶⁷⁴ (CYP2C9) | VI |
| | Kumar 1999⁶⁷⁵ (CYP2C9, | |
| | CYP2C19) | |
| | Kumar 1996⁶⁷⁶ (CYP2C9) | |
| | Eagling 1997⁶⁷⁷ (CYP2C9) | |
| Amprenavir | Fung 2000⁶⁷⁸ (CYP2C9) | VI |
| (PI) | | |
| Saquinavir | Eagling 1997 (ibid) (CYP2C9) | VI |
| (PI) | | |

Cytochrome P450 inducers

| | | |
|---|---|---|
| Nifedipine | Fisslthaler 2000⁶⁷⁹ (CYP2C9) | Krakoff 1993⁶⁸⁰ |
| | | Maccario⁶⁸¹ |
| | | Andronico |
| | | 1991^{682,IV} |

^{I}Increase in BMI was associated with smaller decrease in common femoral arterial stiffness.
^{II}Fluoxetine produces a transient weigh loss leading to gain in body weight in the long term.
^{III}Tolbutamide induced atherosclerosis.
^{IV}Nifedipine reduced insulin resistance.
^{V}Inhibition occurs at supratherapeutic concentrations.
^{VI}Replacing, or not including a protease inhibitor in therapy was associated with attenuated fat distribution abnormalities and insulin resistance (Barreiro 2000⁶⁸³, Mulligan 2000⁶⁸⁴, Gervasoni 1999⁶⁸⁵, Carr 2000⁶⁸⁶, Martinez 2000⁶⁸⁷, see also review, Passalaris 2000⁶⁸⁸).

Drugs are not “empty.” Drugs have other chemical reactions aside from inhibition of CYP-ERK. Take a microcompetition induced clinical symptom, such as weight gain. There are three possible events. The other chemical reactions might increase, decrease or not change body weight. Take the combined effect of CYP-ERK inhibition and the other chemical reactions. The Ho hypothesis assumes a uniform (random) distribution of these events, that is, the probability of every such event is ⅓ so that the probability that a CYP-ERK inhibitor causes weight gain is ⅓. The probability that each of two CYP-ERK different inhibitors cause weight gain is (⅓)*(⅓). In the table above there are 16 drugs, 15 CYP-ERK inhibitors and 1 CYP-ERK inducer. The probability that the 15 inhibitors increase weight and the 1 inducer reduces weight, under the H₀ assumption, is (⅓)¹⁶ or <0.0001.

(2) Mutation, Injury, and Diet Induced Molecular Disruptions

See section on obesity.

7. Discovery 7: Treatment

A healthy system is in stable equilibrium. Microcompetition establishes a new, stable equilibrium, which reflects the modified availability of transcription resources. Assume that the two equilibria are points in a measure space, that is, a space with a unit and direction. In fact, almost all molecular and clinical measurements define such a space. Assume that any point in this space indicates a disease, and that the severity of the disease increases with the distance from the healthy system equilibrium. In this space, the distance between the microcompetition equilibrium and the healthy system equilibrium is small. The small distance between equilibria results in slow progression of the microcompetition diseases. Atherosclerosis or cancer, for instance, may take years to become clinically evident. Consider FIG. 45.

Denote difference between equilibria with A, and denote difference between the microcompetition equilibrium (M_{E}) and the healthy system equilibrium (H_{E}) with Δ(M_{E}−H_{E}). Most successful treatments create a new equilibrium (T_{E}) somewhere between M_{E} and H_{E}. The small distance between the microcompetition equilibrium and the healthy system equilibrium poses a challenge in measuring the effectiveness of such treatments. Since T_{E} is between M_{E} and H_{E}, the distance between T_{E} and M_{E} is even smaller than the distance between H_{E} and M_{E}, Δ(T_{E}−H_{E})<Δ(M_{E}−H_{E}). We assumed that the rate of disease progression/regression, of the microcompetition diseases is a function of the distance between equilibria. Hence, the difference in rate of disease progression between the rate of progression after treatment and during microcompetition is even smaller. Since the clinical changes induced by the move from point H_{E} to M_{E} are usually difficult to measure, the clinical changes induced by the move from point M_{E} to T_{E} are also difficult to measure (most likely even more difficult).

To address this issue, the following sections report results of studies which meet two conditions. One, since treatment effectiveness is reflection of the distance between two states of system equilibrium, only in vivo studies are included. Second, since the effect of treatment is slow to occur, only results of clinical and animal studies conducted over extended periods of time, at least a few weeks, are included. In some cases, the included studies report results which were obtained after years of treatment.

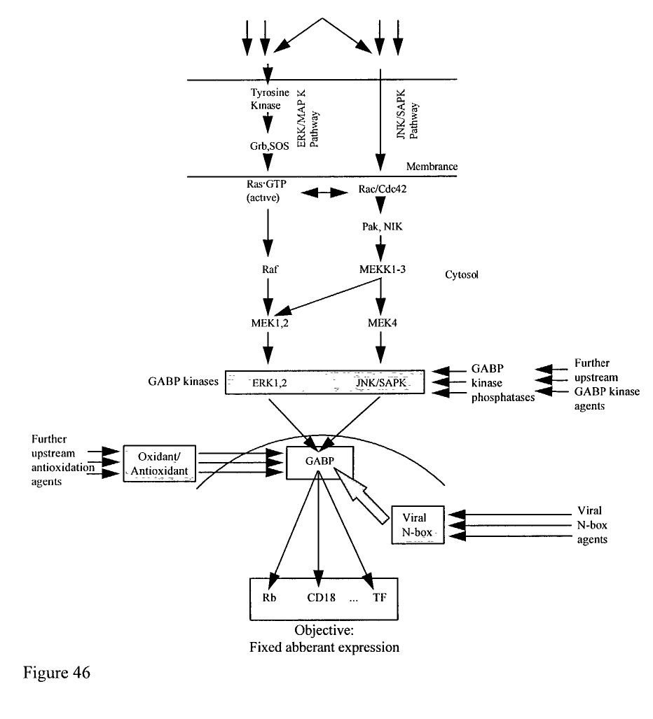

The studies are divided into three sections. The first section includes studies with GABP kinase agents. These agents stimulate the phosphorylation of a GABP kinase, such as ERK or JNK. The second section inludes studies with antioxidation agents. These agents reduce oxidation stress in infected cells. The third section includes studies with viral N-box agents. These agents reduce the concentration of viral DNA in the host. Consider FIG. 46. The targets of these treatments are marked with filled boxes. Microcompetition between viral N-box and ceullar genes for GABP is marked with a thick arrow.

(1) GABP Kinase Agents

A GABP kinase agent stimulates the phosphorylation of a GABP kinase, such as ERK or JNK. The increase in the GABP kinase phosphorylation increases transcription of GABP stimulated genes and decreases transcription of GABP suppressed genes (see above). Since, microcompetition has the opposite effect on these classes of genes, a GABP kinase agent leads to slower progression of the microcompetition diseases.

(a) Dietary Fiber

(i) Effect on Sodium Butyrate

Dietary fiber leads to production of sodium butyrate, a short chain fatty acid (SCFA), during anaerobic fermentation in the colon.

(ii) Effect on ERK

Sodium butyrate is an ERK agent (see above). As a result, sodium butyrate phosphorylates GABP, which, in turn, potentiates binding of p300.

(iii) Effect on Microcompeted Genes

(a) Metallothionein

Microcompetition with a GABP virus decreases expression of metallothionein (see above). Treatment with sodium butyrate activated the metallothionein (MT) gene in certain carcinoma cell lines. Consider the following studies.

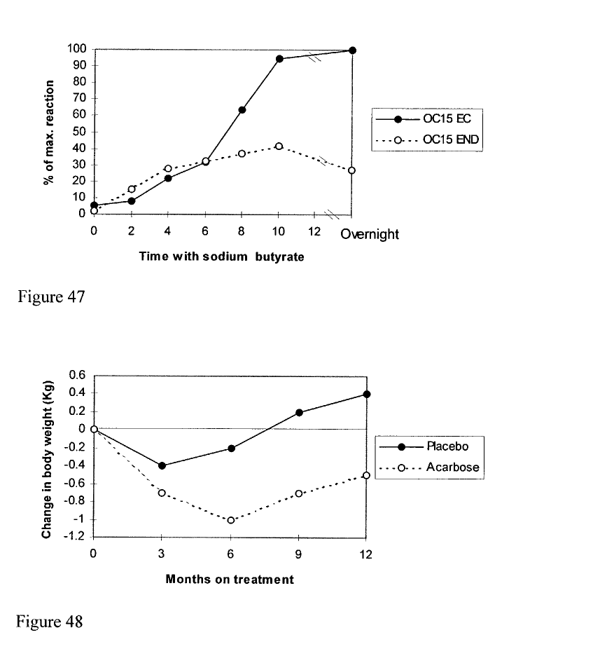

Different embryonal carcinoma cell lines show different basal levels of MT mRNA. For instance, the F9 cell line shows intermediate basal levels of MT expression, while PC 13, a similar cell line, shows very high levels. Since OC15S1 stem cells usually have very low basal levels, these cell were chosen for testing the effect of sodium butyrate on MT mRNA. OC15 embryonal carcinoma (OC15 EC) cells differentiate during 4 days in culture in the presence of retinoic acid (OC15 END). OC15 EC and OC15 END cells were treated with sodium butyrate and the MT mRNA levels were analyzed by Northern blots and quantified by densitometry. FIG. 47 presents the results (Andrews 1987⁶⁸⁹, FIG. 1).

The results show that sodium butyrate increases MT mRNA in both undifferentiated OC15 EC and differentiated OC15 END cells. F9 EC cells, although having higher MT basal mRNA levels, responded similarly to sodium butyrate treatment. It should be noted that the effect of sodium butyrate was specific since sodium propionate and sodium acetate, the other two products of bacterial fermentation in the colon, had no effect on MT mRNA levels.

Another study used ROS 17/2.8, a cloned rat osteosarcoma cell line. In this study, sodium butyrate induced MT synthesis in a dose-dependent manner (Thomas 1991⁶⁹⁰).

A third study used rat primary, non-transformed hepatocytes. Sodium butyrate treatment of these cells produced a 2-4-fold increase in MT mRNA (Liu 1992⁶⁹¹, FIG. 6).

It is interesting that in the non-transformed cells sodium butyrate increased MT mRNA 2-4 fold, while in some carcinoma cell lines the increase was 20 fold (see, for instance, the increase in MT mRNA in OC15 embryonal carcinoma cells above). A compelling explanation is that the relatively low basal MT mRNA in OC15 cells result from microcompetition with viral DNA present in these cells. In such a case, sodium butyrate should show a larger effect in OC15 relative to the non-transformed cells.

(iv) Effect on Clinical Symptoms

(a) Obesity, Insulin Resistance, Hypertension

The Coronary Artery Risk Development in Young Adults (CARDIA) Study, a multicenter population-based cohort study, tested the change in cardiovascular disease (CVD) risk factors over a 10-year period (1985-1986 to 1995-1996) in Birmingham, Ala.; Chicago, Ill.; Minneapolis, Minn.; and Oakland, Calif. A total of 2,909 healthy black and white adults, age 18 to 30 years at enrollment, were included in the study. The results showed that dietary fiber consumption was inversely associated with body weight in both blacks and whites. At all levels of fat intake, subjects consuming the most fiber gained less weight than those consuming the least fiber. Moreover, fiber consumption was also inversely associated with fasting insulin levels and systolic and diastolic blood pressure in both black and white subjects. (Ludwig 1999⁶⁹²).

Fifty-two overweight patients, mean body mass index (BMI)=29.3, participated in a 6 month, randomized, double blind, placebo controlled, parallel group design, study. The treatments included an energy restricted diet plus dietary fiber supplement of 7 g/day, or the diet plus placebo. The results showed that the fiber treated patients lost significantly more weight relative to the placebo treated patients (5.5±0.7 kg, vs. 3.0±0.5 kg, P=0.005). Hunger feelings, measured using visual analogue scales (VAS), were significantly reduced in the fiber-treated group, whereas a significant increase was seen in the placebo group (P<0.02) (Rigaud 1990⁶⁹³).

In another study, ninety-seven mildly obese females participated in 52 week, randomized, double-blind, placebo-controlled trial, study. The treatment consisted of a restricted diet providing 1,200 kcal/day and a dietary fiber supplement of 7 g/day for 11 weeks, (part I), followed by a diet providing 1,600 kcal/day and a dietary fiber supplement of 6 g/day for 16 weeks (part II). Finally placebo was withdrawn and all remaining compliant subjects were given a dietary fiber supplement of 6 g/day and an ad libium diet for the rest of the period (part III). Initial body weights were comparable in the fiber group and placebo group. The results showed that during part I, weight reduction in the fiber supplemented group was significantly higher compared to the placebo group (4.9 kg and 3.3 kg, respectively, P=0.05). Accumulated weight reduction during part II remained significantly higher in the fiber-supplemented group compared to the placebo group (3.8 kg and 2.8 kg, respectively, P<0.05). (Total weight loss in the fiber group after 52 weeks was 6.7 kg). The probability of adherence to the treatment regimen was significantly higher in the fiber group from week 13 and onwards (P<0.01). Initial blood pressures were comparable. A significant reduction of systolic blood pressure was observed in both groups. However, a significant reduction of diastolic blood pressure was observed in the fiber group only (P<0.05) (Ryttig 1989⁶⁹⁴).

These studies show that dietary fiber consumption induces weight loss, reduces insulin resistance and attenuates hypertension.

(b) Atherosclerosis

Soybean hull is a rich source of dietary fiber. Therefore, a diet enriched with soybean hull should attenuate atherosclerosis. Consider the following study.

Twenty five monkeys were divided into 5 groups, each subjected to a different diet. The T1 group received the basal diet; T2, the basal diet plus palm oil; T3, the basal diet plus palm oil and soybean hull; T4, the basal diet plus cholesterol, and T5, the basal diet plus cholesterol and soybean hull. The diets were given for a period of 8 months with water provided ad lib. At the end of the experiment thorax surgery was performed on the animals under general anesthesia. The aorta was removed for histopathological observation and stained with hematoxylin and eosine. Histopathological observation of the aorta showed that adding soybean hull to the basal diet+palm oil diet reduced formation of atherosclerotic lesions from 46.67 of the T1 group to 31.25% in the T3 group. Adding soybean hull to the basal diet+cholesterol reduced formation of lesion from 86.25 to 53.38% (Piliang 1996⁶⁹⁵). Based on these observations, Piliang, et al., concluded that “the soybean hull given in the diet has the ability to prevent the development of atherosclerosis in the aorta of the experimental animals.”

(c) Cancer

Consumption of dietary fiber is associated with reduced risk of several types of cancer (Kim 2000⁶⁹⁶, Madar 1999⁶⁹⁷, Camire 1999⁶⁹⁸, Mohandas 1999⁶⁹⁹, Heaton 1999⁷⁰⁰, Cummings 1999⁷⁰¹, Ravin 1999⁷⁰², Reddy 1999A⁷⁰³, Reddy 1999B⁷⁰⁴, Earnest 1999⁷⁰⁵, Kritchevsky 1999⁷⁰⁶, Cohen 1999⁷⁰⁷).

(b) Acarbose

Acarbose is a α-glucosidase inhibitor, a new class of drugs used in the treatment of diabetes mellitus. α-glucosidases are enzymes released from the brush border of the small intestine. The enzymes hydrolyze di- and oligosaccharides, derived from diet and luminal digestion of starch by pancreatic amylase, into monosaccharides. Since only monosaccharides are transported across intestinal cell membranes, α-glucosidase inhibition reduces carbohydrate absorption.

(i) Effect on Sodium Butyrate

Acarbose inhibits starch digestion in the human small intestine, and therefore, increases the amount of starch available for microbial fermentation to acetate, propionate, and butyrate in the colon. A study examined fermentations by fecal suspensions obtained from subjects who participated in an acarbose-placebo crossover trial. The results showed that the concentrations of acetate, propionate, and butyrate were 57, 13, and 30% of the total final concentrations, respectively, for acarbose treated subjects and 57, 20, and 23% for untreated subjects (Wolin 1999⁷⁰⁸, Table 1, the statistical significance for the difference between acarbose and placebo was P<0.002 for propionate, and P<0.02 for butyrate). Based on these results, Wolin, et al., concluded that “our results show that acarbose treatment results in decreases in the activities of colonic bacteria . . . that form propionate and an increase in the activity of bacteria that produce butyrate.”

To determine the effects of acarbose on colonic fermentation, another study gave subjects 50-200 mg acarbose, or placebo (cornstarch), three times per day, with meals in a double-blind crossover study. Fecal concentrations of starch and starch-fermenting bacteria were measured and fecal fermentation products were determined after incubation of fecal suspensions with and without added substrate for 6 and 24 h. Substrate additions were cornstarch, cornstarch plus acarbose and potato starch. Dietary starch consumption was similar during acarbose and placebo treatment periods. The results showed that butyrate in feces, measured either as concentration or percentage of total short-chain fatty acids, was significantly greater with acarbose treatment compared to placebo, while propionate was significantly smaller (Wolin 1999, ibid, Table 1. P<0.0001). Moreover, butyrate production was significantly greater in fermentations in samples collected during acarbose treatment, whereas production of acetate and propionate was significantly less. Based on their results, Wolin, et al., concluded that “acarbose effectively augmented colonic butyrate production by several mechanisms; it reduced starch absorption, expanded concentrations of starch-fermenting and butyrate-producing bacteria and inhibited starch use by acetate- and propionate-producing bacteria.”

(ii) Effect on Clinical Symptoms

(a) Obesity

Acarbose or placebo was administered to non-insulin dependent diabetes (NIDDM) patients for 1 year in a randomized, double blind, placebo controlled, parallel design study. The effect of acarbose treatment on change in body weight is summarized in FIG. 48 (Wolever 1997⁷⁰⁹, FIG. 1).

After one year, the 130 subjects treated with acarbose each experienced an average weight loss of 0.46±0.28 kg. In contrast, the 149 subject treated with placebo each experienced a 0.33±0.25 kg weight gain (P=0.027). Interestingly, acarbose had no effect on energy intakes, nutrient intakes, or dietary patterns.

(c) Vanadate

An ERK phosphatase is an enzyme that inactivates ERK by dephosphorylation of either Thy, Tyr, or both residues (see above). The class of all ERK phosphatases includes, for instance, PP2A, a type 1/2 serine/threonine phosphatase, PTP1B, a protein tyrosine phosphatase, and MKP-1, a dual specificity phosphatase. Inhibition of an ERK phosphatase stimulates ERK phosphorylation. The increase in ERK phosphorylation increases transcription of GABP stimulated genes and decreases transcription of GABP suppressed genes (see above). Since, microcompetition has the opposite effect on these classes of genes, inhibition of an ERK phosphatase leads to slower progression of the microcompetition diseases. Consider vanadate as an example.

(i) Effect on PTP

Vanadate (VO₄⁻³) and vanadate derivatives are general protein tyrosine phosphatase (PTP) inhibitors. Specifically, vanadate, and pervanadate (a general term for the variety of complexes formed between vanadate and hydrogen peroxide) were shown to inhibit the protein-tyrosine phosphatase PTP1B (Huyer 1997⁷¹⁰).

(ii) Effect on ERK

PTPs dephosphorylate and deactivate ERK (see above). As general PTP inhibitors, vanadate and vanadate derivatives are expected to activate ERK, an observation reported in several studies (Wang 2000⁷¹¹, Zhao 1996⁷¹², Pandey 1995⁷¹³, D'Onofrio 1994⁷¹⁴).

(iii) Effect on GABP Regulated Genes

(a) F-type PFK-2/FBPase-2 is GABP Stimulated Gene

The bifunctional enzyme 6-phosphofructo-2-kinase (EC 2.7.1.105, PFK-2)/fructose-2,6-bisphosphatase (EC 3.1.3.46 FBPase-2) catalyzes the synthesis and degradation of fructose-2,6-bisphosphate. The rat PFK-2/FBPase-2 gene (gene A) codes for the fetal (F), muscle (M), and liver (L) mRNAs. Each of these mRNAs originates from a different promoter in the gene. The F-type promoter includes an enhancer in the (−1809-1615) region with three N-boxes at (−1747-1742), (−1716-1710) and (−1693-1688) (Darville 1992⁷¹⁵, FIG. 4). The enhancer stimulated transcription, especially in FTO2B hepatoma cells (Ibid, Table 1). DNase I protection experiments using the enhancer and extracts from FTO2B cell, from C2C12 myoblasts or myocytes, or from liver, but not from muscle, showed one specific footprint corresponding to the middle N-box (Ibid, FIG. 5). Gel retardation assays with extracts from FTO2B and HTC cells, L6 myoblasts and myocytes, and liver, but not muscle, showed a major complex (Ibid, FIG. 6A). When this enhancer fragment was methylated at single purines using dimethylsulfate and subsequently incubated with FTO2B extracts, three contact points were detected within the N-box (Ibid, FIG. 4). The three points of methylation interference coincide with contact points identified by the same technique in the two N-boxes of the adenovirus E1A core enhancer which binds GABP. A subsequent study (Dupriez 1993⁷¹⁶) showed that changing the GG, essential for ets DNA binding, to CC in both distal and proximal N-boxes decreased promoter activity by 15-20%. Changing GG to CC in the middle N-box decreased promoter activity by 75%. The study also showed that anti-GABPα and anti-GABPβ antibodies inhibited formation of complexes on the middle N-box by FTO2B proteins (Ibid, FIG. 4, lane 5 and 6). Transfection with recombinant GABPα and GABPβ produced shifts that comigrated with these complexes and were inhibited by anti-GABPα antibodies (Ibid, FIG. 4, lane 12-16). These observations suggest that the F-type PFK-2/FBPase-2 is a GABP stimulated gene.

A GABP virus microcompetes with the F-type PFK-2/FBPase-2 enhancer for GABP. Therefore, viral infection of cells decreases F-type PFK-2/FBPase-2 expression. Moreover, higher concentration of viral DNA results in greater decrease in F-type PFK-2/FBPase-2 expression.

(b) Vanadate Stimulates F-Type PFK-2/FBPase-2 transcription

ERK activation is expected to stimulate transcription of GABP stimulated genes. The rat F-type PFK-2/FBPase-2 gene is a GABP stimulated gene. Therefore, vanadate should stimulate transcription of F-type PFK-2/FBPase-2. Consider the following studies.

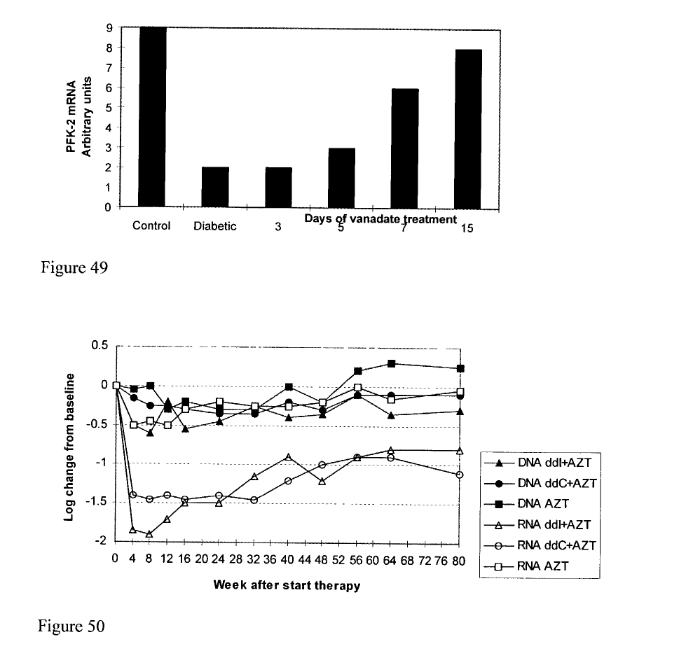

The effect of sodium orthovanadate by oral administration on liver PFK-2/FBPase-2 mRNA content was measured in rats with streptozotocin (STZ)-induced diabetes. The mRNA content was measured after 3, 5, 7 and 15 days of treatment. The results are presented in FIG. 49 (Miralpeix 1992⁷¹⁷, FIG. 3).

Vanadate treatment of diabetic animals produced a progressive increase in liver PFK-2/FBPase-2 mRNA content, reaching a nearly normal level after 15 days. Inoue (1994⁷¹⁸) reports similar results.

The F-type PFK-2/FBPase-2 is usually not expressed in liver cells. However, the F-type mRNA levels increase in proliferating cells. Dupriez, et al., (1993, ibid) measured tissue expression of the gene. F-type PFK-2/FBPase-2 mRNA was present in hepatoma, fibroblast, and myoblasts cell lines. The mRNA was found in fetal liver and muscle, the two fetal tissues examined. In adult tissues the mRNA was found in the lung and thymus. In the other adult tissues tested the mRNA was present at much lower concentrations or was undetectable. The highest concentration was in preterm placenta, with a decrease at term. The concentration decreased upon differentiation of L6 myoblasts into myocytes (Ibid, FIG. 2) and in Rat-1 fibroblasts made quiescent by lowering serum concentration in culture from 10 to 0.1%. Moreover, F-type mRNA concentration increased in FTO2B cells upon dexamethasone treatment. Based on these observations, Dupreiz, et al., concluded that the “expression of the F-type mRNA appears to correlate with cell proliferation.”

Usually, liver tissue shows limited cell proliferation. However, in the Miralpeix 1992 study (see above), vanadate was administred to male Sprague-Dawley rats one week after the animals were treated with a single intravenous injection of streptozotocin (STZ). As it turns out, STZ injection to Sprague-Dawley rats induces high levels of hepatocyte proliferation. Consider the following study.

Hepatocyte proliferation was measured in Sprague-Dawley rats made diabetic by iv injection of STZ. The results showed a 12% increase in the ratio of liver weight to body weight in diabetic rats 8 days after injection compare to normal rats, and a 44% increase at 30 days (Herrman 1999⁷¹⁹). The results also showed an increase in hepatocyte mitosis to 300% of normal at 8 days, a return to normal at 30 days, and a decrease to 25% of normal at 90 days (Ibid, FIG. 1). Based on these results Herrman, et al., concluded that “hepatomegaly observed in streptozotocin-induced experimental diabetes may be due primarily to early hyperplasia.”

The Miralpeix 1992 study used a “1.4 kilobase rat liver PFK-2/FBPase-2 cDNA probe which corresponds to the mRNA for liver PFK-2/FBPase-2 devoid of the 5′ end coding for amino acids 1-90.” This probe does not distinguish between F-type and L-type PFK-2/FBPase-2 mRNA. Therefore, the reported increase in PFK-2/FBPase-2 mRNA is, most likely, a result of the increase in F-type PFK-2/FBPase-2 mRNA in hepatocytes induced to proliferate by a streptozotocin injection.

(iv) Effect on Clinical Symptoms

(a) Obesity

Five week-old Zucker rats, an animal model of obesity and insulin resistance, were divided into three groups of 6 rats: lean (Fa/fa) control, obese (fa/fa) control and obese (fa/fa)-vanadate treated. The rats in the treated group received sodium orthovanadate through drinking water for four months. Obese rats had significantly higher body weight compared to lean controls. However, body weight of vanadate-treated obese decreased 43% to levels comparable to lean controls (Pugazhenthi 1995⁷²⁰, Table 1).

McNeill and Orvig (1996⁷²¹) report similar results. Wistar rats were divided into two groups, control (8 animals) and treated (11 animals). Treated animals recieved between 0.3 and 0.5 mmol/kg of bis(maltolato)oxovanadium/day in drinking water over a 77 day period. Beginning at day 56 the treated animals showed reduced weight gain compared 722 to controls (Ibid, FIG. 1, group 2 vs. group 1). (See also Dai 1994, and Bhanot 1994⁷²³.)

(b) Cancer

Cruz, et al., (1995)⁷²⁴ tested the antineoplastic effect of orthovanadate on a subcutaneous MDAY-D2 tumor mouse model. Ten week old DBA/2j female mice were injected sucutaneously in the posterior lateral side with 4×10⁵ cells in 100 μl of PBS. On day 5, the mice were divided into two groups. One group received subcutaneous injections of 100 μl of PBS and another group received 100 μl of PBS containing 500 μg of orthovanadate daily. The orthovanadate was administrated subcutaneously on the opposite, tumor-free, posterior lateral side. On day 14, the mice were sacrificed, weighed and tumors were resected and weighed. The results showed decreased tumor growth in treated mice compared to controls (Ibid, FIG. 6). In control mice, the tumor weights varied from 0.86-1.74 g, whereas in orthovanadate treated mice, four mice showed no detectable tumors and 11 mice showed tumors varying from 0.08-0.47 g. Orthovanadate treatment reduced tumor growth by more than 85%, sometimes completely inhibiting tumor formation.

Another study tested the chemoprotective effect of vanadium against chemically induced hepatocarcinogenesis in rats. Initiation was performed by a single intraperitoneal injection of diethylnitrosamine (DENA; 200 mg kg⁻¹) followed by promotion with phenobarbital (0.05%) in diet. Vanadium (0.5 ppm) was provided ad libitum throughout the experiment in drinking water. The results showed that after 20 weeks vanadium reduced the incidence (P<0.01), total number and multiplicity (P<0.001), and altered the size distribution of visible persistent nodules (PNs) as compared with DENA controls (Bishayee and Chatterjee 1995⁷²⁵). Mean nodular volume (P<0.05) and nodular volume as a percent of liver volume (P<0.01) were also attenuated. Vanadium also caused a large decrease in number (P<0.001) and surface area (P<0.01) of gamma-glutamyltranspeptidase (GGT)-positive hepatocyte foci and in labeling index (P<0.001) of focal cells, coupled with increased (P<0.01) remodeling. The activity of GGT, measured quantitatively, was found to be significantly less in PNs (P<0.001) and non-nodular surrounding parenchyma (P<0.01) of vanadium-supplemented rats. Histopathological analysis of liver sections showed well-maintained hepatocellular architecture compared to DENA control. Based on these results, Bishayee and Chatterjee (1995) concluded that “our results, thus, strongly suggest that vanadium may have a unique anti-tumor potential.”

See also Liasko 1998⁷²⁶.

(c) Diabetes

Numerous in vivo studies demonstrated reduced blood glucose in insulin deficient diabetic animals, and improved glucose homeostasis in obese, insulin-resistant diabetic animals, following treatment with vanadate. In human studies, insulin sensitivity improved in NIDDM patients and in some IDDM patient after treatment with vanadate (see recent reviews Goldfine 1995⁷²⁷, Brichard 1995⁷²⁸).

As an example consider the study by Pugazhenthi, et al., (1995, see above). This study also tested the effect of vanadate on diabetes. The obese Zucker rats showed elevated plasma levels of glucose and insulin. Vanadate treatment decreased plasma glucose and insulin levels by 36% and 80%, respectively (Ibid, Table 1).

(d) PTP1B Knockout

(i) Effect on PTP and ERK

Gene knockout is a special case of intervention. The result of a PTP1B gene knockout is PTP1B enzyme deficiency. Vanadate inhibits PTP1B (Huyer 1997, ibid). Therefore, both PTP1B gene knockout and adminstration of vanadate result in reduced activity of the PTP1B enzyme. Considering the discussion above, the PTP1B gene knochout effect on clinical symtoms should be similar to the effects of vanadate treatment.

(ii) Effect on Clinical Symptoms

(a) Obesity

A targeting vector was designed to delete a segment of the mouse homolog of the PTP1B gene. This segment included exon 5 and the tyrosine phosphatase active site in exon 6. The deleted segments were replaced with the neomycin resistance gene. Two separate embryonic stem cell clones that had undergone homologous recombination and possessed a single integration event were microinjected Balb/c blastocyts. Chimeric males were mated with wild-type Balb/c females, and heterozygotes from this cross were mated to product animals homozygous for the PTP1B mutation (Elchebly 1999⁷²⁹, FIG. 1A). The PTP1B protein was absent in PTP1B null mice (PTP1B(−/−)), and heterozygotes (PTP1B(+/−)) expressed about half the amount of PTP1B relative to wild type mice (Ibid, FIG. 1B). PTP1B null mice grew normally on regular diet, did not show any significant difference in weight gain compared to wild-type mice and lived longer than 1.5 years without any signs of abnormality and were fertile. To study the effect of PTP1B gene knockout on obesity, PTP1B(−/−), PTP1B(+/−) and wild type mice were fed a high-fat diet normally resulting in obesity. As expected, the wild-type mice rapidly gained weight. In contrast, the PTP1B(−/−), PTP1B(+/−) mice were protected from the diet induced weight gain (Ibid, FIG. 5). Based on these results, Elchebly, et al., concluded that PTP1B deficiency results in obesity resistance.

Another study reported results of a PTP1B gene disruption. Klaman, et al., (2000⁷³⁰) generated PTP1B-null mice by targeted disruption of the ATG coding exon (exon 1). The PTP1B-deficient mice showed low adiposity and protection from diet-induced obesity. The decreased adiposity resulted from reduced fat cell mass without a decrease in adipocyte number. Leanness in PTP1B-deficient mice was associated with increased basal metabolic rate and total energy expenditure.

(b) Diabetes

Elchebly, et al., (1999, ibid) also tested the effect of PTP1B gene knockout on diabetes. In the fed state, PTP(−/−) mice given a regular diet showed a 13% reduction and PTP(+/−) a 8% reduction in blood glucose concentration relative to wild type mice (Ibid, FIG. 2A). Fed PTP1B(−/−) mice on regular diet had circulating insulin levels of about half of wild type fed animals (Ibid, FIG. 2B). The enhanced insulin sensitivity of the PTP1B(−/−) mice was also observed in glucose and insulin tolerance tests (Ibid, FIGS. 3A and 3B). The PTP1B(−/−), PTP1B(+/−) and wild type mice were also fed a high-fat diet normally resulting in insulin resistant. As expected, the wild-type mice became insulin resistance. In contrast, on a high-fat diet the PTP1B(−/−) mice showed glucose and insulin concentrations similar to animals on normal diet (Ibid, Table 1). PTP1B(−/−) mice also showed enhanced insulin sensitivity relative to wild type in both glucose and insulin tolerance tests (Ibid, FIGS. 6A, 6B). On high-fat diet, the PTP1B(+/−) mice showed increased fasting concentrations of circulating insulin but similar fasting glucose concentrations relative to animals on normal diet (Ibid, Table 1). Based on these results, Elchebly, et al, concluded that PTP1B deficiency results in enhanced insulin sensitivity. The PTP1B-deficient mice in Klaman, et al., (2000⁷³¹) showed similar enhanced insulin-stimulated whole-body glucose disposal.

As expected, both a PTP1B deficiency and vanadate treatment result in resistance to obesity and enhanced insulin sensitivity. We speculate that PTP1B gene knockout, in a manner similar to vanadate treatment, also induces cancer resistance.

(2) Antioxidants

Microcompetition and oxidative stress both decrease binding of GABP to the N-box. Therefore, microcompetition can be viewed as “excessive oxidative stress.” Some antioxidants reduce intracellular oxidative stress. These antioxidants stimulate the binding of GABP to the N-box thereby attenuating the effect of microcompetition on transcription, resulting in slower progression of the microcompetition diseases.

(a) Garlic

(i) Effect on Oxidative Stress

Garlic is a scavenger of free radicals. A study investigated, using high pressure liquid chromatography, the ability of unheated or heated garlic extract to scavenge hydroxyl radical (.OH) generated by photolysis of H₂O₂ (1.2-10 μmoles/ml) with ultraviolet (UV) light and trapped with salicylic acid (500 mmoles/ml). H₂O₂ produced .OH in a concentration-dependent manner as estimated by the .OH adduct products 2,3-dihydroxybenzoic acid (DHBA) and 2,5-DHBA. Garlic extract (5-100 μl/ml) inhibited (30-100%) 2,3-DHBA and 2,5-DHBA production in a concentration-dependent manner (Prasad 1996⁷³², FIG. 3). Garlic activity was reduced by 10% approximately, when heated to 100 degrees C. for 20, 40 or 60 min. Garlic extract also prevented the .OH-induced formation of malondialdehyde (MDA) in rabbit liver homogenate in a concentration-dependent manner (Ibid, FIG. 10). In the absence of .OH, garlic did not affect MDA levels. Based on these results, Pasas, et al., (1996) concluded that “garlic extract is a powerful scavenger of .OH.”

Another study examined the antioxidant effects of garlic extract in a cellular system using bovine pulmonary artery endothelial cells (PAEC) and murine macrophages (J774). The study used intracellular glutathione (GSH) depletion as an index of oxidative stress. Oxidized LDL (Ox-LDL) caused a depletion of GSH. Pretreatment with aged garlic extract inhibited Ox-LDL induced peroxides in PAEC and suppressed peroxides in macrophages in a dose-dependent manner (Ide 1999⁷³³). In a cell free system, the aged garlic extract was shown to scavenge H₂O₂ similarly. These results together show that aged garlic extract prevents the Ox-LDL-induced depletion of GSH in endothelial cells and macrophages.

(ii) Effect on Clinical Symptoms

(a) Atherosclerosis

Garlic attenuates the formation of atherosclerotic plaque. A study involved the de-endothelialization of the right carotid artery of 24 rabbits by balloon catheterization in order to produce myointimal thickening. After 2 weeks the rabbits were randomly assigned to four groups: Group I received a standard diet (standard); Group II received standard diet supplemented with 800 μl/kg body weight/day of the aged garlic extract “Kyolic” (standard+Kyolic); Group III received a standard diet supplemented with 1% cholesterol (cholesterol-enriched); and Group IV received standard diet supplemented with 1% cholesterol and Kyolic (cholesterol-enriched+Kyolic). After 6 weeks, the cholesterol-enriched diet caused a 6-fold increase in serum cholesterol levels (Group III) compared to standard diet (Group I) (P<0.05) (Efendy 1997⁷³⁴, FIG. 1). At 6 weeks, the cholesterol-enriched diet (Group III) showed fatty streak lesions covering approximately 70±8% of the surface area of the thoracic aorta. The cholesterol-enriched+Kyolic group (Group IV) showed fatty lesions in only 25±3% of the same surfce area (Ibid, FIGS. 2A and 2B), which represents a reduction of about 64%. No lesions were present in Groups I and II. The cholesterol-enriched diet also caused an increase in aortic arch cholesterol (2.1±0.1 mg cholesterol/g tissue), which was significantly reduced by Kyolic (1.7±0.2 mg cholesterol/g tissue) (P<0.05). Kyolic significantly inhibited the development of thickened, lipid-filled lesions in the pre-formed neointimas produced by balloon-catheter injury of the right carotid artery in cholesterol-fed rabbits (intima as percent of artery wall, Group III 42.6±6.5% versus Group IV 23.8±2.3%, P<0.01). Kyolic had little effect in rabbits on a standard diet (Group 1118.4±5.0% versus Group I 16.7±2.0%). In vitro studies showed that Kyolic inhibited smooth muscle proliferation (Ibid, FIG. 5). Based on these results, Efendy, et al., (1997) concluded that “Kyolic treatment reduces fatty streak development, vessel wall cholesterol accumulation and the development of fibro fatty plaques in neointimas of cholesterol-fed rabbits, thus providing protection against the onset of atherosclerosis.”

Jain (1978⁷³⁵), Jain (1976⁷³⁶) and Bordia (1975⁷³⁷) reported similar observations. Jain (1978) and Jain (1976) used rabbits fed a 16 week standard or cholesterol-enriched diet supplemented with or without garlic extract. In both studies the results showed marked atherosclerotic lesions in animals fed a cholesterol-enriched diet relative to standard diet. The animals fed a cholesterol-enriched diet supplemented with garlic extract showed attenuated lesion formation. Jain (1978) also reported reduced aorta cholesterol content in garlic treated animals. Bordia (1975) used rabbits fed for 3 months on similar diets. The results showed that garlic attenuated the formation of atherosclerotic plaque and the increase in lipid content of aorta.

Garlic treatment resulted in other favorable effects associated with attenuated atherosclerosis. A study measured the elastic properties of the aorta using pulse wave velocity (PWV) and pressure-standardized elastic vascular resistance (EVR) techniques. The subjects included healthy adults (n=101; age 50 to 80 years) who were taking 300 mg/d or more of standardized garlic powder for at least 2 years and 101 age- and sex-matched controls. Blood pressure, heart rate, and plasma lipid levels were similar in the two groups. The results showed that PWV (8.3±1.46 versus 9.8±2.45 m/s; P<0.0001) and EVR (0.63±0.21 versus 0.9±0.44 m²·s⁻²·mm Hg⁻¹; P<0.0001) were lower in the garlic group than in the control group (Breithaupt-Grogler 1997⁷³⁸, Table 1, FIG. 1). PWV showed significant positive correlation with age (garlic group, r=0.44; control group, r=0.52, FIG. 3) and systolic blood pressure (SBP) (garlic group, r=0.48; control group, r=0.54, FIG. 4). With any degree of increase in age or SBP, PWV increased less in the garlic group than in the control group (P<0.0001, FIG. 3, FIG. 4). ANCOVA and multiple regression analyses demonstrated that age and SBP were the most important determinants of PWV and that the effect of garlic on PWV was independent of confounding factors. According to Breithaupt-Grogler, et al., (1997), “The data suggested that the elastic properties of the aorta were maintained better in the garlic group that in the control group.” It is interesting that in experimental animals, changes of ratio of intimal (plaque) area to medial area during progression and regression of atherosclerosis correlated with changes in indices of aortic elastic properties. Pregression of atherosclerosis resulted in higher PWV, and vice versa (Farrar 1991⁷³⁹).

See also studies in the special supplement of the British Journal of Clinical Practice (1990, Supplement 69) dedicated to the clinical effects of garlic in ischemic heart disease.

Microcompetition increases the transcription of P-selectin in endothelial cells, increases the transcription of tissue factor (TF) and decreases the transcription of β₂ integrin and α₄ integrin in macrophages and decreases the transcription of retinoblastoma susceptibility gene (Rb) in smooth muscle cells (SMC). Garlic reduces oxidative stress in endothelial cells, macrophages and SMCs. The reduced oxidative stress stimulates the binding of GABP to these genes, decreasing the transcription of TF and P-selectin and increasing the transcription of β₂ integrin, α₄ integrin and Rb. A change in transcription of these genes attenuates the formation of atherosclerotic plaque and thickening of the aortic intima.

(b) Cancer

The anticancer properties of garlic were recognized thousands of years ago. The ancient Egyptians used garlic externally for treatment of tumors. Hippocrates and physicians in ancient India are also reported to have used garlic externally for cancer treatment. Recent studies confirmed these properties. See, for instance, the section “Garlic, Onions and Cancer,” in the recent review by Ali, et al., (2000⁷⁴⁰), the meta-analysis of the epidemiologic literature on garlic consumption and the risk of stomach and colon cancer (Fleischauer 2000⁷⁴¹), and specific animals studies demonstrating garlic suppression of chemically induced tumors (Singh 1998⁷⁴², Singh 1996⁷⁴³).

(3) Viral N-Box Agents

A viral N-box agent reduces the number of active viral N-boxes in the host cell. The reduction can be accomplished by an overall reduction in the copy number of viral genomes present, or by inhibition of viral N-boxes (for instance by antisense), etc. The reduced number of active viral N-boxes eases microcompetition and consequently slows progression of the microcompetition diseases.

(a) Direct Antiviral Agents

(i) Ganciclovir

(a) Effect on viral DNA elongation

Ganciclovir (Cytovene, DHPG) is a guanosine analogue. The prodrug is phosphorylated by thymidine kinase to the active triphosphate form after uptake into the infected cell. The triphosphate form inhibits viral DNA polymerase by competing with cellular deoxyguanosine triphosphate for incorporation into viral DNA causing chain termination. Ganciclovir is effective against herpes simplex virus 1 and 2 (HSV-1, HSV-2), cytomegalovirus (CMV), Epstein-Barr virus (EBV) and varicella-zoster virus (Spector 1999⁷⁴⁴).

Aciclovir (acyclovir) and its oral form valacyclovir, and penciclovir and it oral form famciclovir are guanosine analogues similar to ganciclovir. These drugs are also effective against HSV-1, HSV-2 and CMV. See, for instance, a recent meta-analysis of 30 aciclovir clinical trials in HSV infections (Leflore 2000⁷⁴⁵), a review on aciclovir recommended treatments in HSV infections (Kesson 1998⁷⁴⁶), reviews on valaciclovir effectiveness in HSV and CMV infections (Ormord 2000⁷⁴⁷, Bell 1999⁷⁴⁸) and a review of famciclovir and penciclovir (Sacks 1999⁷⁴⁹).

(b) Effect on Latent Viral DNA Load

The load of viral DNA during latent infection is directly correlated with the extent of viral replication during the preceding productive infection (Reddehase 1994⁷⁵⁰, Collins 1993⁷⁵¹). Therefore, reduction of viral replication should reduce the load of viral DNA during a subsequent latent infection. Consider the following studies. Bone marrow transplantation (BMT) was performed as a syngeneic BMT with female BALB/c (H-2^{d}) mice used at the age of 8 weeks as both bone marrow donors and recipients. Two hours after BMT, the mice were infected subcutaneously in the left hind footpad with murine CMV. The mice were than divided into four groups. Three groups received therapy with increasing doses of CD8 T cells. The fourth groups served as controls. The results showed that increasing doses of CD8 T cells significantly reduced the extent and duration of virus replication in vital organs, such as lungs and adrenal glands (Steffens 1998⁷⁵², FIG. 2). Moreover, 12 months after BMT, the viral DNA load was measured. The results showed that the amount of DNA was smaller in the groups given CD8 T cell therapy. The viral DNA load in the lungs of mice given no immunotherapy was 5,000 viral genomes per 10⁶ lung cells. The load following treatment with 10⁵ and 10⁶ CD8 T cells was 3,000 and 1,000 per 10⁶ lung cells, respectively. Since there were no infectious virus present, the study shows that attenuated viral replication during the acute phase of infection reduces the load of viral DNA during the subsequent latent phase of infection.

The study also measured the recurrence of viral infection following therapy. Five latently infected mice with no therapy and five mice treated with 10⁷ CD8 T cells were subjected to immunoablative 7-ray treatment of 6.5 Gy. Recurrence of viral infectivity was measured 14 days later in separate lobes of the lungs. The group receiving no therapy showed a high latent DNA load and recurrence of infectivity in all five mice in all five lobes of the lungs (with some variance). In contrast, the group receiving CD8T cells showed low viral load and recurrence of infectivity in only two mice and only in a single lobe in each mouse (Steffens 1998, FIG. 7). These results show that a reduction in viral replication reduces latent viral DNA load and the probability viral disease.

Thackary and Field, in a series of studies, also tested the effect of preemptive therapy against viral infection. However, instead of CD8 T cells, the studies administered famciclovir (FCV), valaciclovir (VACV), or human immunoglobulin (IgG) to mice infected via the ear pinna or the left side of the neck with either HSV-1 or HSV-2 (Thackray 2000A⁷⁵³, Thackray 2000B⁷⁵⁴, Thackray 2000C⁷⁵⁵, Field 2000⁷⁵⁶, Thackray 1998⁷⁵⁷). The results showed that 9-10 days of FCV treatment early in infection was effective in limiting the establishment of viral latency several months after treatment. Based on their results, Field and Thackary conclude that “Thus, the implication of our results is that even intensive antiviral therapy starting within a few hour of exposure is unlikely to compeletly abrogate latency. However, our results also show a significant reduction in the number of foci that are established and imply that there may also be a quantitative reduction in the latent genomes.” (Field 2000, ibid).

Another study compared the effect of aciclovir (ACV) and immunoglobulin (IgG) preemptive therapy on mice infected via scarified corneas with HSV-1. Both therapies were administered for 7 days commencing on the first day post infection. The results showed that ACV treatment resulted in a reduced copy number of latent HSV-1 genome on day 44 post infection relative to IgG (LeBlanc 1999⁷⁵⁸, FIG. 5). Since no untreated mice survived the infection, the study could not compare ACV treatment to no treatment. However, if we assume that IgG treatment either reduced or did not change the copy number of latent viral genomes, we can conclude that the ACV preemptive treatment resulted in a reduced load of latent viral DNA.

Ganciclovir is similar to aciclovir and penciclovir. Therefore, a reasonable conclusion from these studies is that preemptive treatment with ganciclovir will also reduce the load of viral DNA.

(c) Effect on Clinical Symptoms

(i) Atherosclerosis

Accelerated coronary atherosclerosis can be observed in the donor heart following heart transplantation (TxCAD). Transplanting a heart from a CMV seropositive donor to a seronegative recipient increases the probability of a primary infection in the recipient (Bowden 1991⁷⁵⁹ Chou 1988⁷⁶⁰, Chou 1987⁷⁶¹, Chou 1986⁷⁶², Grundy 1988⁷⁶³, Grundy 1987⁷⁶⁴, Grundy 1986⁷⁶⁵). The Thackary and LeBlanc studies demonstrated that administration of aciclovir or penciclovir prophylaxis early in primary infection reduces the load of the subsequent latent viral DNA in the infected animals (see above). Since microcompetition between viral and cellular DNA results in atherosclerosis, prophylactic administration of ganciclovir, a drug similar to aciclovir and penciclovir, early after heart transplantation, should reduce atherosclerosis. Consider the following study.

One hundred and forty-nine consecutive patients (131 men and 18 women, aged 48±13 years) randomly received either ganciclovir or placebo. The study drug was commenced on the first postoperative day and was administered for 28 days. In 22% of patients drug administration was delayed by up to 6 days due to acute-care problems. Immunosuppression consisted of muromonab-CD3 (OKT-3) prophylaxis and maintenance with cyclosporine, prednisone, and azathioprine. Coronary angiography was performed annually after heart transplantation. Mean follow-up time was 4.7±1.3 years. TxCAD was defined as the presence of any angiographic disease irrespective of severity because of the recognized underestimation of TxCAD by angiography. The actuarial incidence of TxCAD was determined from these annual agiograms and from autopsy data. CMV infection was determined in recipient and donor. The results showed that actuarial incidence of TxCAD at follow-up was 43±8% in patients treated with ganciclovir compared with 60±11% in placebo group (P<0.1). Moreover, the protective effect of ganciclovir was even more evident when the population of CMV seronegative recipients was considered exclusively. Of the 14 CMV seronegative recipients randomized to prophylactic ganciclovir, 4 (28%), developed TxCAD compared with 9 (69%) of the seronegative patients randomized to placebo (Valantine 1999⁷⁶⁶). The effect of ganciclovir is less evident in the population as a whole since among seropositive recipients there was no difference between ganciclovir and placebo. TxCAD developed in 22 (47%) of 48 patients randomized to ganciclovir compared with 21 (47%) of 46 in the placebo group. Base on these results, Valantine, et al., concluded that “prophylactic treatment with ganciclovir initiated immediately after heart transplantation reduces the incidence of TxCAD.”

It is interesting to note that in a multivariate analysis, the study found that the variable “CMV illness” was not an independent predictor of TxCAD when “lack of ganciclovir” and “donor age” were included in the analysis. We suspect that high correlation (multicollinearity) between “lack of ganciclovir” and “CMV illness” produced this result. Such a correlation was demonstrated in numerous studies. See, for instance, table 5 in Sia (2000⁷⁶⁷), which lists 10 clinical studies showing that early administration of ganciclovir prophylaxis in solid-organ transplantation resulted in reduced CMV disease compared to no treatment, administration of placebo, treatment with immunoglobulin or treatment with acyclovir. From this correlation we deduce that Valantine (1999) also measured reduced CMV disease (the study is mute on this statistic). The key parameter that determines the overall and organ-specific risks of CMV disease is the copy number of latent viral genomes in various tissues (Reddehase 1994, ibid). Therefore, the reduced CMV disease indicates a reduction in the copy number of latent viral genome, which, again, explains the reduction in observed atherosclerosis.

(ii) Zidovudine (AZT), Didanosine (ddI), Zalcitabine (ddC)

(a) Effect on Viral DNA Elongation

Didanosine (2′,3′-dideoxyinosine, ddI) is a synthetic purine nucleoside analogue used against HIV infection. After passive diffusion into the cell, the drug undergoes phosphorylation by cellular (rather than viral, see above) enzymes to dideoxyadenosine-5′-triphosphate (ddATP), the active moiety. ddATP competes with the natural substrate for HIV-1 reverse transcriptase (deoxyadenosine 5′-triphosphate) and cellular DNA polymerase. Because ddATP lacks the 3′-hydroxyl group present in the naturally occurring nucleoside, incorporation into viral DNA leads to termination of DNA chain elongation and inhibition of viral DNA growth (see a recent review of ddI in Perry 1999⁷⁶⁸).

Zidovudine (retrovir, ZDV, AZT) and zalcitabine (ddC) are nucleosides similar to ddI.

(b) Effect on Latent Viral DNA Load

A study measured the change in HIV-1 DNA and RNA load relative to baseline in 42 antiretroviral naive HIV-1 infected persons treated with either AZT monotherapy, a combination of AZT+ddC or a combination of AZT+ddI over a period of 80 weeks. FIG. 50 presents the results (Breisten 1998⁷⁶⁹, FIG. 1A).

At week 80, AZT treatment alone was associated with an increase, ddC+AZT with a small decrease and ddI+AZT with a larger decrease in viral DNA. To compare the results statistically, the mean log change from baseline over all time points was compared between ddI+AZT and ddC+AZT. The mean change was −0.3375 and −0.20458 for ddI+AZT and ddC+AZT, respectively (P=0.02). It is interesting that, although not significant statistically (P=0.29), rank order of the ddI+AZT and ddC+AZT effect on RNA is reversed, that is, the mean effect of ddC+AZT on viral RNA was larger than ddI+AZT. Since the combination therapy of AZT and ddC is additive (Magnani 1997⁷⁷⁰), the ddC monotherapy effect on viral DNA was calculated as the ddC+AZT effect minus the AZT monotherapy effect. The calculated effect of ddC monotherapy on viral DNA was compared to the effect of AZT monotherapy. The mean log change from baseline over all time points was −0.15458 and −0.05 for ddC and AZT, respectively (P=0.09). The statistical analysis suggests that the ranking of ddI>ddC>AZT in terms of their effect on viral DNA, is significant. Moreover, the results suggest that at later time points, AZT tend to be associated with increased levels of viral DNA.

This statistical analysis is different from the analysis reported by Bruisten, et al (1986). To test whether an “early” response occurred, Bruisten, et al., averaged the values of weeks 4, 8, and 12 and for a “late” response the values of weeks 32, 40, and 48. The test showed that only the ddI+AZT treatment decreased the HIV-1 viral DNA “early” and “late.” The P value of “early” compared to baseline is 0.002, the p value of “late” compare to baseline is 0.052. The same values for ddC+AZT are 0.191 and 0.08. These values also indicate that ddI is more effective than ddC in reducing viral DNA.

Another study (Pauza 1994⁷⁷¹) measured the total viral DNA by polymerase chain reaction (PCR) assays for viral LTR sequences in 51 HIV infected patients. This assay detects linear, circular, and integrated HIV-1 DNA and also includes preintegration complexes that completed the first translocation step. Twenty patients were treated with AZT, 4 patients with ddI and 7 patients with ddC. After Southern blotting and hybridization, fragments were excised from the membrane and bound radioactivity was determined by scintillation counting. The measured LTR DNA levels were expressed on a scale of 1 to 5 (1 is lowest). Negative samples were labeled zero. The average ranking of viral DNA load for patients treated with ddI, ddC and AZT, was 2.25, 2.71 and 2.74, respectively. The difference between ddC and AZT is small. However, the average CD4 μl count for ddC and AZT treated patients was 82 and 191.55, respectively (p<0.03 for the difference). Hence, the viral DNA load of the AZT group is most likely biased downward. Overall, this ranking of treatment effectiveness measured in terms of reduced viral DNA load is identical to the ranking in Breisten 1998 above.

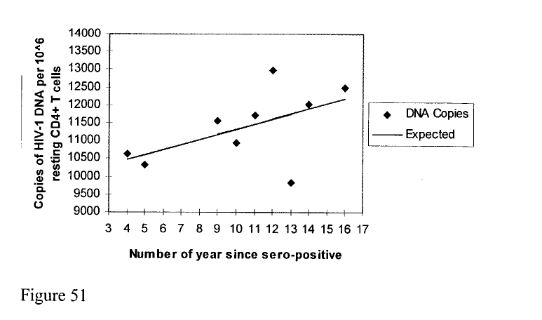

A third study (Chun 1997⁷⁷²) measured total HIV-1 DNA in 9 patients. Eight patients were on triple therapy including two nucleosides and one protease inhibitor. One patient received two nucleosides and two protease inhibitors. Six patients had undetectable plasma HIV RNA. The other three patients had 814, 2,800 and 6,518 copies/ml. The study also reports the year of seroconversion. A regression analysis with viral DNA level as dependent variable and number of years since seroconversion as independent variable produces the results shown in FIG. 51:

Viral DNA load=9,909+142×Years since seroconversion

The viral DNA load is measured in copies of HIV-1 DNA per 10⁶ resting CD4+ T cells. The p values for the intercept and coefficient are 1.31 E-05 and 0.131481, respectively. Since the sample size is small, the p value for the coefficient is considered as borderline significant, which means that even with triple and quadruple therapies, and in patients with mostly undetectable plasma HIV RNA, viral DNA load increases with an increase in the number of years since seroconversion.

The difference between the expected and the observed number of viral DNA copies was calculated for each patient. The therapy of two patients included ddI and the average difference for these patients was −828 copies. The therapy of five patients included AZT and the average difference for these patients was +317 copies. These results suggest that ddI is associated with a decrease and AZT with an increase in the number of viral DNA copies in this group of patients.

Under different conditions, with monotherapy, triple and quadruple therapy with a protease inhibitor and with detectable and undetectable RNA, the results are consistent. ddI is associated with a larger reduction in viral DNA load compared to ddC, and AZT is associated with an increase in viral DNA load.

(c) Effect on Clinical Symptoms

(i) Obesity

A study observed 306 six HIV-infected women between December 1997 and February 1998 (Gervasoni 1999, ibid). The women were treated with two or more antiretroviral drugs. One hundred and sixty two patients were treated with two nucleosides (double therapy) and 144 with three or more drugs including at least one protease inhibitor (PI) (triple therapy). Fat redistribution (FR) was confirmed by means of a physical examination and dual-energy X-ray absorptiometry (DEXA). FR was observed in 32 women (10.5%) (12 on double therapy, 20 on triple therapy). The body changes were reported to gradually emerge over a period of 12-72 weeks. A statistical analysis showed that a combination treatment which included ddI was significantly associated with the absence of FR(P=0.019). A combination treatment which included ddC was also significantly associated with the absence of FR(P=0.049). The p values indicate that a ddI-including combination was more effective than a ddC-including therapy in preventing FR. Contrary to ddI and ddC, a combination therapy which included AZT was associated with a low risk of developing FR(OR 0.3).

The association between ddI-, ddC- and AZT-including therapeutic combinations with fat redistribution is consistent with their effect on reducing or increasing viral DNA load.

Another interesting observation in this study was that the longer median total duration of antiretroviral drug treatment in women with FR compared to those without FR (1,187 versus 395 days). Only one of the 32 women with FR received antiretroviral drug therapy for less than 1,000 days. The risk of FR for women under antiretroviral drug therapy for more than 1,000 days was 10 times greater than in those who received shorter drug therapy (OR 10.8, P=0.0207).

A statistical analysis of the results in Chun 1997 (see above) showed that viral DNA load increases with an increase in the number of years since seroconversion. Since the duration of antiretroviral drug treatment most often increases with the number of years since seroconversion, longer duration correlates with higher viral DNA load. Higher viral DNA load results in more intense microcompetition, and therefore, fat redistribution.

(iii) Garlic

(a) Effect on Viral Infectivity

Garlic has antiviral activity. See for instance Guo, et al., (1993⁷⁷³) and Weber, et al., (1992⁷⁷⁴).

(b) Effect on Clinical Symptoms

See abve.

(b) Immune Stimulating Agents

The balance between two forces, the virus drive to replicate, and the capacity of the immune system to control or clear the infection, determines the copy number of viral genome present in infected cells. A stable equilibrium between these two forces determines the copy number in persistent and latent infections. A major determinant of the immune system capacity to clear or control and infection is the efficiency of the Th1 response. An increase in this efficiency reduces the viral copy number.

(i) Infection with Non-GABP Viruses

Data obtained in animals indicate that neonatal immune responses are biased toward Th2. Consider the effects of a productive infection with a GABP virus during early life. The extent of viral replication during productive infection determines the load of viral DNA during the subsequent latent infection (see discussion above). The lower the Th1 efficiency during the productive infection, the higher the copy number of viral genome in the subsequent latent period. Infection with some viruses, such as measles, hepatitis A, and Mycobacterium tuberculosis induce a strong polarized Th1-type response in early life. These infections reduce GABP virus replication and subsequent genome copy number during latent infection. The reduced copy number attenuates microcompetition, therefore, reducing the probability and severity of microcompetition diseases, such as, atopy, asthma, diabetes, cancer, atherosclerosis, osteoarthritis, obesity, etc. Consider the following studies.

BCG is a freeze-dried preparation made from a living culture of the Calmette-Guerin strain of mycobacterium Bovis. It was first developed as a vaccine against tuberculosis in 1921 but also has been used as an immunotherapeutic treatment for carcinoma. Vaccination with BCG induces a Th 1-type immune response in human newborn and adults human (Marchant 1999⁷⁷⁵). Moreover, BCG immunization prior to challenge with herpes simplex virus increased survival rate of newborn mice (Starr 1976⁷⁷⁶). To investigate whether the prevalence of atopy is lower in children who have been vaccinated with BCG in infancy than in children who have not been vaccinated, a study measured skin test reactivity to three allergens (Dermatophagoides pteronyssinus, D. farinae and cockroach) in 400 children, aged 3-14 years, in an urban area of Bissau, the capital of Guinea-Bissau in west Africa. The results showed that 57 (21%) of the vaccinated children were atopic (any reaction > or =2 mm), compared with 21 (40%) of the unvaccinated children [odds ratio, after controlling for potential confounding factors, 0.19 (95% CI 0.06-0.59)]. When atopy was defined using the 3-mm criterion, the reduction in atopy associated with BCG was greater the earlier the age at vaccination, and the largest reduction was seen in children vaccinated in the first week of life (Aaby 2000⁷⁷⁷). Based on these results, Aaby, et al., concluded that “BCG vaccination given early in infancy may prevent the development of atopy in African children.”

Results of numerous studies suggest that measles, hepatitis A, and Mycobacterium tuberculosis infection in early life may prevent subsequent development of atopic diseases. In humans, immunomodulation during the first two years of life is most successful in producing long-lasting prevention effects (von Hertzen 2000⁷⁷⁸). See also von Mutisu 2000⁷⁷⁹, von Hertzen 1999⁷⁸. As a result of this observed effect, there are currently attempts to use BCG as a vaccine for asthma (see review Scanga 2000⁷⁸¹).

A study evaluated the protective effect of repeated BCG vaccinations on preventing diabetes in NOD mice. The results showed that 17/32 (53%) of the control group, 8/31 (26%) of the single vaccine-treated (at age 35 days) mice, and 7/23 (30%) of the single vaccine-treated (at age 90 days) mice developed diabetes, and none of the repeated BCG vaccination (at age 35 & 90 days, n=14) animals developed the disease, up to 250 days of age (p<0.05, compared with controls and each of the single-vaccination groups). The repeated BCG vaccination reduced the severity of insulitis at age 120 days as compared with controls and single BCG-vaccination groups (Shehadeh 1997⁷⁸²). On the relation between BCG immunization and type 1 diabetes, see also Qin 1997⁷⁸³, Harada 1990⁷⁸⁴ and a recent review Hiltunen 1999⁷⁸⁵.

Another study showed that an infection of NOD mice with Mycobacterium avium, before the mice show overt diabetes, results in permanent protection of the animals from diabetes. This protective effect was associated with increased numbers of CD4+ T cells and B220+ B cells (Martins 1999⁷⁸⁶). The study also showed that the protection was associated with changes in the expression of Fas (CD95) and FasL by immune cells, and alterations in cytotoxic activity, IFNγ and IL-4 production and activation of T cells of infected animals. Based on these results, Martins and Aguas concluded that the “data indicate that protection of NOD mice from diabetes is a Th1-type response that is mediated by up-regulation of the Fas-FasL pathway and involves an increase in the cytotoxicity of T cells.” See also Bras 1996⁷⁸⁷.

(ii) Breast-Feeding

Breast-feeding increases the efficiency of the Th1 immune response. Consider the following studies.

A study measured the blast transformation and cytokine production by lymphocytes, and T cell changes of 59 formula-fed and 64 breast fed 12-month-old children blast, before and after measles-mumps-rubella vaccination (MMR). The results showed that before vaccination, lymphocytes of breast fed children had lower levels of blast transformation without antigen (p<0.001), with tetanus toxoid (p<0.02) or Candida (p<0.04), and lower IFNγ production (p<0.03). Fourteen days after live viral vaccination, only breast fed children had increased production of IFNγ (p<0.02) and increased percentages of CD56+ (p<0.022) and CD8+ cells (p<0.004) (Pabst 1997⁷⁸⁸). Based on these results, Pabst, et al., concluded that “these findings are consistent with a Th1 type response by breast fed children, not evident in formula-fed children. Feeding mode has an important long-term immunomodulating effect on infants beyond weaning.” See also the review Pabst 1997⁷⁸⁹.

Another study showed immunophenotypic differences between breast-fed and formula-fed infants consistent with accelerated development of immune system in breast-fed infants (Hawkes 1999⁷⁹⁰).

Since breast-feeding increases the efficiency of the Th1 immune response, it should reduce the probability and severity of microcompetition diseases (see above for detail). Consider the following studies.

A study examined the association between breast-feeding and type II diabetes (also called non-insulin-dependent diabetes, or NIDDM) in Pima Indians, a population with a high prevalence of this disorder. Data were available for 720 Pima Indians aged between 10 and 39 years. 325 people who were exclusively bottle fed had significantly higher age-adjusted and sex-adjusted mean relative weights (146%) than 144 people who were exclusively breast fed (140%) or 251 people who had some breast-feeding (139%) (p=0.019). The results showed that people who were exclusively breast fed had significantly lower rates of NIDDM than those who were exclusively bottle fed in all age-groups. The odds ratio for NIDDM in exclusively breast fed people, compared with exclusively bottle fed, was 0.41 (95% CI 0.18-0.93) adjusted for age, sex, birth date, parental diabetes, and birth weight (Pettitt 1997⁷⁹¹). Based on these results Pettitt, et al., concluded that “exclusive breast-feeding for the first 2 months of life is associated with a significantly lower rate of NIDDM in Pima Indians.”

Another study measured the impact of breast-feeding on overweight and obesity in children at school entry was assessed in a cross sectional study in Bavaria in 1997. The school entry health examination enrolled 134,577 children. Data on early feeding were collected in two rural districts (eligible population n=13,345). The analyses were confined to 5 or 6 year old children with German nationality. The study measured overweight (BMI>90th percentile for all German children seen at the 1997 school entry health examination in Bavaria) and obesity (BMI>97th percentile). Information on breast-feeding was available for 9,206 children of whom 56% had been breast-fed for any length of time. The results showed that in non breast-fed children the upper tail of the BMI distribution was enlarged as compared to the breast-fed children whereas the median was almost identical (von Kries 2000⁷⁹²). The prevalence of obesity in children who had never been breast-fed was 4.5% as compared to 2.8% in ever breast-fed children. A clear dose response effect for the duration of breast-feeding on the prevalence of obesity was found: 3.8%, 2.3%, 1.7% and 0.8% for exclusive breast-feeding for up to 2, 3 to 5, 6 to 12 and more than 12 months, respectively. The results for overweight were very similar. The protective effect of beast feeding on overweight and obesity could not be explained by differences in social class or lifestyle. The adjusted odds ratios of breast-feeding for any length of time was 0.71 (95% CI 0.56-0.90) for obesity and 0.77 (95%CI 0.66-0.88) for overweight. This data set did not allow adjustments for maternal weight, an important risk factor for obesity in children. Maternal overweight, however, could not explain the effect of breast-feeding on overweight and obesity in a similar study. The reduction in the risk for overweight and obesity is therefore more likely to be related to the properties of human milk than to factors associated with breast-feeding. See also von Kries 1999⁷⁹³.

### TABLE OF CITED REFERENCES

¹ Molecular target drug discovery for cancer: exploratory grants, Natlaonal Cancer Institute, Feb. 16, 2000, http://grants.nih.gov/grants/guide/pa-files/PAR-00-060.html

² Kamei Y, Xu L, Heinzel T, Torchia J, Kurokawa R, Gloss B, Lin S C, Heyman R A, Rose D W, Glass C K, Rosenfeld M G. A CBP integrator complex mediates transcriptional activation and AP-1 inhibition by nuclear receptors. Cell. May 3, 1996;85(3):403-14.

³ Hottiger M O, Felzien L K, Nabel G J. Modulation of cytokine-induced HIV gene expression by competitive binding of transcription factors to the coactivator p300. EMBO J. Jun. 1, 1998;17(11):3124-34.

⁴ Gonelli A, Boccia S, Boni M, Pozzoli A, Rizzo C, Querzoli P, Cassai E, Di Luca D. Human herpesvirus 7 is latent in gastric mucosa. J Med Virol. 2001 April;63(4):277-83.

⁵ Smith R L, Morroni J, Wilcox C L. Lack of effect of treatment with penciclovir or acyclovir on the establishment of latent HSV-1 in primary sensory neurons in culture. Antiviral Res. 2001 October;52(1):19-24.

⁶ Young L S, Dawson C W, Eliopoulos A G. The expression and function of Epstein-Barr virus encoded latent genes. Mol Pathol. 2000 October;53(5):238-47.

⁷ Vo N, Goodman R H. CREB-binding protein and p300 in transcriptional regulation. J. Biol. Chem. Apr. 27, 2001;276(17):13505-8.

⁸ Blobel G A. CREB-binding protein and p300: molecular integrators of hematopoietic transcription. Blood. Feb. 1, 2000;95(3):745-55.

⁹ Goodman R H, Smolik S. CBP/p300 in cell growth, transformation, and development. Genes Dev. Jul. 1, 2000;14(13):1553-77.

¹⁰ Hottiger M O, Nabel G J. Viral replication and the coactivators p300 and CBP. Trends Microbiol. 2000 December;8(12):560-5.

¹¹ Giordano A, Avantaggiati M L. p300 and CBP: partners for life and death. J Cell Physiol. 1999 November;181(2):218-30.

¹² Eckner R. p300 and CBP as transcriptional regulators and targets of oncogenic events. Biol. Chem. 1996 November;377(11):685-8.

¹³ Kitabayashi I, Yokoyama A, Shimizu K, Ohki M. Interaction and functional cooperation of the leukemia-associated factors AML1 and p300 in myeloid cell differentiation. EMBO J. Jun. 1, 1998;17(11):2994-3004.

¹⁴ Facchinetti V, Loffarelli L, Schreek S, Oelgeschlager M, Luscher B, Introna M, Go. Regulatory domains of the A-Myb transcription factor and its interaction with the CBP/p300 adaptor molecules. Biochem J. Jun. 15, 1997;324 (Pt 3):729-36.

¹⁵ Duyndam M C, van Dam H, Smits P H, Verlaan M, van der Eb A J, Zantema A. The N-terminal transactivation domain of ΔTF2 is a target for the co-operative activation of the c-jun promoter by p300 and 12S E1A. Oncogene. Apr. 8, 1999; 18(14):2311-21.

¹⁶ Yukawa K, Tanaka T, Tsuji S, Akira S. Regulation of transcription factor C/ΔTF by the cAMP signal activation in hippocampal neurons, and molecular interaction of C/ΔTF with signal integrator CBP/p300. Brain Res Mol Brain Res. May 21, 1999;69(1):124-34.

¹⁷ Mink S, Haenig B, Klempnauer K H. Interaction and functional collaboration of p300 and C/EBPbeta. Mol Cell Biol. 1997 November;17(11):6609-17.

¹⁸ Yanagi Y, Masuhiro Y, Mori M, Yanagisawa J, Kato S. p300/CBP acts as a coactivator of the cone-rod homeobox transcription factor. Biochem Biophys Res Commun. Mar. 16, 2000;269(2):410-4.

¹⁹ Lamprecht C, Mueller C R. D-site binding protein transactivation requires the proline- and acid-rich domain and involves the coactivator p300. J. Biol. Chem. Jun. 18, 1999;274(25):17643-8.

²⁰ Marzio G, Wagener C, Gutierrez M I, Cartwright P, Helin K, Giacca M. E2F family members are differentially regulated by reversible acetylation. J Biol Chem Apr. 14, 2000;275(15): 10887-92.

²¹ Silverman E S, Du J, Williams A J, Wadgaonkar R, Drazen J M, Collins T. cAMP-response-element-binding-protein-binding protein (CBP) and p300 are transcriptional co-activators of early growth response factor-1 (Egr-1). Biochem J. Nov. 15, 1998;336 (Pt 1):183-9.

²² Kim M Y, Hsiao S J, Kraus W L. A role for coactivators and histone acetylation in estrogen receptor alpha-mediated transcription initiation. EMBO J. Nov. 1, 2001;20(21):6084-94.

²³ Wang C, Fu M, Angeletti R H, Siconolfi-Baez L, Reutens A T, Albanese C, Lisanti M P, Katzenellenbogen B S, Kato S, Hopp T, Fuqua S A, Lopez G N, Kushner P J, Pestell R G. Direct acetylation of the estrogen receptor alpha hinge region by p300 regulates transactivation and hormone sensitivity. J. Biol. Chem. May 25, 2001;276(21): 18375-83.

²⁴ Speir E, Yu Z X, Takeda K, Ferrans V J, Cannon R O 3rd. Competition for p300 regulates transcription by estrogen receptors and nuclear factor-kappaB in human coronary smooth muscle cells. Circ Res. Nov. 24, 2000;87(11):1006-11.

²⁵ Kobayashi Y, Kitamoto T, Masuhiro Y, Watanabe M, Kase T, Metzger D, Yanagisawa J, Kato S. p300 mediates functional synergism between AF-1 and AF-2 of estrogen receptor alpha and beta by interacting directly with the N-terminal A/B domains. J Biol Chem May 26, 2000;275(21):15645-51.

²⁶ Papoutsopoulou S, Janknecht R. Phosphorylation of ETS transcription factor ER81 in a complex with its coactivators CREB-binding protein and p300. Mol Cell Biol. 2000 October;20(19):7300-10.

²⁷ Jayaraman G, Srinivas R, Duggan C, Ferreira E, Swaminathan S, Somasundaram K, Williams J, Hauser C, Kurkinen M, Dhar R, Weitzman S, Buttice G, Thimmapaya B. p300/cAMP-responsive element-binding protein interactions with ets-1 and ets-2 in the transcriptional activation of the human stromelysin promoter. J. Biol. Chem. Jun. 11, 1999;274(24):17342-52.

²⁸ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.

²⁹ Bhattacharya S, Michels C L, Leung M K, Arany Z P, Kung A L, Livingston D M. Functional role of p35srj, a novel p300/CBP binding protein, during transactivation by HIF-1. Genes Dev. Jan. 1, 1999;13(1):64-75.

³⁰ Kallio P J, Okamoto K, O'Brien S, Carrero P, Makino Y, Tanaka H, Poellinger L. Signal transduction in hypoxic cells: inducible nuclear translocation and recruitment of the CBP/p300 coactivator by the hypoxia-inducible factor-1 alpha. EMBO J. Nov. 16, 1998;17(22):6573-86.

³¹ Ema M, Hirota K, Mimura J, Abe H, Yodoi J, Sogawa K, Poellinger L, Fujii-Kuriyama Y. Molecular mechanisms of transcription activation by HLF and HIF1 alpha in response to hypoxia: their stabilization and redox signal-induced interaction with CBP/p300. EMBO J. Apr. 1, 1999;18(7):1905-14.

³² Soutoglou E, Papafotiou G, Katrakili N, Talianidis I. Transcriptional activation by hepatocyte nuclear factor-1 requires synergism between multiple coactivator proteins. J. Biol. Chem. Apr. 28, 2000;275(17):12515-20.

³³ Yoneyama M, Suhara W, Fukuhara Y, Fukuda M, Nishida E, Fujita T. Direct triggering of the type I interferon system by virus infection: activation of a transcription factor complex containing IRF-3 and CBP/p300. EMBO J. Feb. 16, 1998;17(4):1087-95.

³⁴ Sato S, Roberts K, Gambino G, Cook A, Kouzarides T, Goding C R. CBP/p300 as a co-factor for the Microphthalmia transcription factor. Oncogene. Jun. 26, 1997; 14(25):3083-92.

³⁵ Sartorelli V, Huang J, Hamamori Y, Kedes L. Molecular mechanisms of myogenic coactivation by p300: direct interaction with the activation domain of MyoD and with the MADS box of MEF2C. Mol Cell Biol. 1997 February; 17(2):1010-26.

³⁶ Garcia-Rodriguez C, Rao A. Nuclear factor of activated T cells (NFAT)-dependent transactivation regulated by the coactivators p300/CREB-binding protein (CBP). J Exp Med. Jun. 15, 1998;187(12):2031-6.

³⁷ Sisk T J, Gourley T, Roys S, Chang C H. MHC class II transactivator inhibits IL-4 gene transcription by competing with NF-AT to bind the coactivator CREB binding protein (CBP)/p300. J. Immunol. Sep. 1, 2000;165(5):2511-7.

³⁸ Li Q, Herrler M, Landsberger N, Kaludov N, Ogryzko V V, Nakatani Y, Wolffe A P. Xenopus NF-Y pre-sets chromatin to potentiate p300 and acetylation-responsive transcription from the Xenopus hsp70 promoter in vivo. EMBO J. Nov. 2, 1998;17(21):6300-15.

³⁹ Faniello M C, Bevilacqua M A, Condorelli G, de Crombrugghe B, Maity S N, Avvedimento V E, Cimino F, Costanzo F. The B subunit of the CAAT-binding factor NFY binds the central segment of the Co-activator p300. J. Biol. Chem. Mar. 19, 1999;274(12):7623-6.

⁴⁰ Gerritsen M E, Williams A J, Neish A S, Moore S, Shi Y, Collins T. CREB-binding protein/p300 are transcriptional coactivators of p65. Proc Natl Acad Sci U S A. Apr. 1, 1997;94(7):2927-32.

⁴¹ Iannone M A, Consler T G, Pearce K H, Stimmel J B, Parks D J, Gray J G. Multiplexed molecular interactions of nuclear receptors using fluorescent microspheres. Cytometry Aug. 1, 2001;44(4):326-37.

⁴² Kodera Y, Takeyama K, Murayama A, Suzawa M, Masuhiro Y, Kato S. Ligand type-specific interactions of peroxisome proliferator-activated receptor gamma with transcriptional coactivators. J Biol Chem Oct. 27, 2000;275(43):33201-4.

⁴³ Han B, Liu N, Yang X, Sun H B, Yang Y C. MRG1 expression in fibroblasts is regulated by Sp1/Sp3 and an Ets transcription factor. J. Biol. Chem. Mar. 16, 2001;276(11):7937-42.

⁴⁴ Avantaggiati M L, Ogryzko V, Gardner K, Giordano A, Levine A S, Kelly K. Recruitment of p300/CBP in p53-dependent signal pathways. Cell. Jun. 27, 1997;89(7):1175-84.

⁴⁵ Van Orden K, Giebler H A, Lemasson I, Gonzales M, Nyborg J K. Binding of p53 to the KIX domain of CREB binding protein. A potential link to human T-cell leukemia virus, type I-associated leukemogenesis. J. Biol. Chem. Sep. 10, 1999;274(37):26321-8.

⁴⁶ Yang W, Hong Y H, Shen X Q, Frankowski C, Camp H S, Leff T. Regulation of transcription by AMP-activated protein kinase: phosphorylation of p300 blocks its interaction with nuclear receptors. J Biol Chem Oct. 19, 2001;276(42):38341-4.

⁴⁷ Janknecht R, Wells N J, Hunter T. TGF-beta-stimulated cooperation of smad proteins with the coactivators CBP/p300. Genes Dev. Jul. 15, 1998;12(14):2114-9.

⁴⁸ Feng X H, Zhang Y, Wu R Y, Derynck R. The tumor suppressor Smad4/DPC4 and transcriptional adaptor CBP/p300 are coactivators for smad3 in TGF-beta-induced transcriptional activation. Genes Dev. Jul. 15, 1998;12(14):2153-63.

⁴⁹ de Caestecker M P, Yahata T, Wang D, Parks W T, Huang S, Hill C S, Shioda T, Roberts A B, Lechleider R J. The Smad4 activation domain (SAD) is a proline-rich, p300-dependent transcriptional activation domain. J Biol Chem Jan. 21, 2000;275(3):2115-22.

⁵⁰ Pearson K L, Hunter T, Janknecht R. Activation of Smadl-mediated transcription by p300/CBP. Biochim Biophys Acta Dec. 23, 1999;1489(2-3):354-64.

⁵¹ Pouponnot C, Jayaraman L, Massague J. Physical and functional interaction of SMADs and p300/CBP. J. Biol. Chem. Sep. 4, 1998;273(36):22865-8.

⁵² Oliner J D, Andresen J M, Hansen S K, Zhou S, Tjian R. SREBP transcriptional activity is mediated through an interaction with the CREB-binding protein. Genes Dev Nov. 15, 1996; 10(22):2903-11.

⁵³ Paulson M, Pisharody S, Pan L, Guadagno S, Mui A L, Levy D E. Stat protein transactivation domains recruit p300/CBP through widely divergent sequences. J. Biol. Chem. Sep. 3, 1999;274(36):25343-9.

⁵⁴ Zhang J J, Vinkemeier U, Gu W, Chakravarti D, Horvath C M, Darnell J E Jr. Two contact regions between Stat1 and CBP/p300 in interferon gamma signaling. Proc Natl Acad Sci U S A. Dec. 24, 1996;93(26): 15092-6.

⁵⁵ Bhattacharya S, Eckner R, Grossman S, Oldread E, Arany Z, D'Andrea A, Livingston D M. Cooperation of Stat2 and p300/CBP in signalling induced by interferon-alpha. Nature. Sep. 26, 1996;383(6598):344-7.

⁵⁶ Pfitzner E, Jahne R, Wissler M, Stoecklin E, Groner B. p300/CREB-binding protein enhances the prolactin-mediated transcriptional induction through direct interaction with the transactivation domain of Stat5, but does not participate in the Stat5-mediated suppression of the glucocorticoid response. Mol Endocrinol. 1998 October; 12(10):1582-93.

⁵⁷ Gingras S, Simard J, Groner B, Pfitzner E. p300/CBP is required for transcriptional induction by interleukin-4 and interacts with Stat6. Nucleic Acids Res. Jul. 1, 1999;27(13):2722-9.

⁵⁸ Hamamori Y, Sartorelli V, Ogryzko V, Puri P L, Wu H Y, Wang J Y, Nakatani Y, Kedes L. Regulation of histone acetyltransferases p300 and PCAF by the bHLH protein twist and adenoviral oncoprotein E1A. Cell Feb. 5, 1999;96(3):405-13.

⁵⁹ Manning E T, Ikehara T, Ito T, Kadonaga J T, Kraus W L. p300 forms a stable, template-committed complex with chromatin: role for the bromodomain. Mol Cell Biol. 2001 June;21(12):3876-87.

⁶⁰ Kraus W L, Manning E T, Kadonaga J T. Biochemical analysis of distinct activation functions in p300 that enhance transcription initiation with chromatin templates. Mol Cell Biol. 1999 December;19(12):8123-35.

⁶¹ Kraus W L, Kadonaga J T. p300 and estrogen receptor cooperatively activate transcription via differential enhancement of initiation and reinitiation. Genes Dev. Feb. 1, 1998;12(3):331-42.

⁶² Rosmarin A G, Luo M, Caprio D G, Shang J, Simkevich C P. Sp1 Cooperates with the ets Transcription Factor, GABP, to Activate the CD18 (β₂ Leukocyte Integrin) Promoter. Journal of Biological Chemistry 1998 273(21): 13097-13103.

⁶³ Avots A, Hoffmeyer A, Flory E, Cimanis A, Rapp U R, Serfling E. GABP factors bind to a distal interleukin 2 (IL-2) enhancer and contribute to c-Raf-mediated increase in IL-2 induction. Molecular and Cellular Biology 1997 17(8):4381-4389.

⁶⁴ Lin J X, Bhat N K, John S, Queale W S, Leonard W J. Characterization of the human interleukin-2 receptor beta-chain gene promoter: regulation of promoter activity by ets gene products. Mol Cell Biol. 1993 October;13(10):6201-10.

⁶⁵ Markiewicz S, Bosselut R, Le Deist F, de Villartay J P, Hivroz C, Ghysdael J, Fischer A, de Saint Basile G. Tissue-specific activity of the gammac chain gene promoter depends upon an Ets binding site and is regulated by GA-binding protein. J. Biol. Chem. Jun. 21, 1996;271(25):14849-55.

⁶⁶ Smith M F Jr, Carl V S, Lodie T, Fenton M J. Secretory interleukin-1 receptor antagonist gene expression requires both a PU.1 and a novel composite NF-kappaB/PU.1/GA-binding protein binding site. J. Biol. Chem. Sep. 11, 1998;273(37):24272-9.

⁶⁷ Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. Aug. 1, 1997;57(15):3145-8.

⁶⁸ Kamura T, Handa H, Hamasaki N, Kitajima S. Characterization of the human thrombopoietin gene promoter. A possible role of an Ets transcription factor, E4TF1/GABP. J. Biol. Chem. Apr. 25, 1997;272(17):11361-8.

⁶⁹ Wang K, Bohren K M, Gabbay K H. Characterization of the human aldose reductase gene promoter. J. Biol. Chem. Jul. 25, 1993;268(21):16052-8.

⁷⁰ Nuchprayoon I, Shang J, Simkevich C P, Luo M, Rosmarin A G, Friedman A D. An enhancer located between the neutrophil elastase and proteinase 3 promoters is activated by Sp] and an Ets factor. J. Biol. Chem. Jan. 8, 1999;274(2):1085-91.

⁷¹ Nuchprayoon I, Simkevich C P, Luo M, Friedman A D, Rosmarin A G. GABP cooperates with c-Myb and C/EBP to activate the neutrophil elastase promoter. Blood. Jun. 15, 1997;89(12):4546-54.

⁷² Sadasivan E, Cedeno M M, Rothenberg S P. Characterization of the gene encoding a folate-binding protein expressed in human placenta. Identification of promoter activity in a G-rich SP1 site linked with the tandemly repeated GGAAG motif for the ets encoded GA-binding protein. J. Biol. Chem. Feb. 18, 1994;269(7):4725-35.

⁷³ Basu A, Park K, Atchison M L, Carter R S, Avadhani N G. Identification of a transcriptional initiator element in the cytochrome c oxidase subunit Vb promoter which binds to transcription factors NF-E1 (YY-1, delta) and Sp1. J. Biol. Chem. Feb. 25, 1993;268(6):4188-96.

⁷⁴ Sucharov C, Basu A, Carter R S, Avadhani N G. A novel transcriptional initiator activity of the GABP factor binding ets sequence repeat from the murine cytochrome c oxidase Vb gene. Gene Expr. 1995;5(2):93-111.

⁷⁵ Carter R S, Avadhani N G. Cooperative binding of GA-binding protein transcription factors to duplicated transcription initiation region repeats of the cytochrome c oxidase subunit IV gene. J. Biol. Chem. Feb. 11, 1994;269(6):4381-7.

⁷⁶ Carter R S, Bhat N K, Basu A, Avadhani N G. The basal promoter elements of murine cytochrome c oxidase subunit IV gene consist of tandemly duplicated ets motifs that bind to GABP-related transcription factors. J. Biol. Chem. Nov. 15, 1992;267(32):23418-26.

⁷⁷ Virbasius J V, Scarpulla R C. Activation of the human mitochondrial transcription factor A gene by nuclear respiratory factors: a potential regulatory link between nuclear and mitochondrial gene expression in organelle biogenesis. Proc Natl Acad Sci U S A. Feb. 15, 1994;91(4):1309-13.

⁷⁸ Villena J A, Vinas O, Mampel T, Iglesias R, Giralt M, Villarroya F. Regulation of mitochondrial biogenesis in brown adipose tissue: nuclear respiratory factor-2/GA-binding protein is responsible for the transcriptional regulation of the gene for the mitochondrial ATP synthase beta subunit. Biochem J. Apr. 1, 1998;331 (Pt 1):121-7.

⁷⁹ Ouyang L, Jacob K K, Stanley F M. GABP mediates insulin-increased prolactin gene transcription. J. Biol. Chem. May 3, 1996;271(18): 10425-8.

⁸⁰ Hoare S, Copland J A, Wood T G, Jeng Y J, Izban M G, Soloff M S. Identification of a GABP alpha/beta binding site involved in the induction of oxytocin receptor gene expression in human breast cells, potentiation by c-Fos/c-Jun. Endocrinology. 1999 May;140(5):2268-79.

⁸¹ Mantovani R. A survey of 178 NF-Y binding CCAAT boxes. Nucleic Acids Res. Mar. 1, 1998;26(5):1135-43.

⁸² Espinos E, Le Van Thai A, Pomies C, Weber M J. Cooperation between phosphorylation and acetylation processes in transcriptional control. Mol Cell Biol 1999 May;19(5):3474-84.

⁸³ Shiraishi M, Hirasawa N, Kobayashi Y, Oikawa S, Murakami A, Ohuchi K. Participation of mitogen-activated protein kinase in thapsigargin- and TPA-induced histamine production in murine macrophage RAW 264.7 cells. Br J Pharmacol 2000 February;129(3):515-24.

⁸⁴ Herrera R, Hubbell S, Decker S, Petruzzelli L. A role for the MEK/MAPK pathway in PMA-induced cell cycle arrest: modulation of megakaryocytic differentiation of K562 cells. Exp Cell Res Feb. 1, 1998;238(2):407-14.

⁸⁵ Stadheim T A, Kucera G L. Extracellular signal-regulated kinase (ERK) activity is required for TPA-mediated inhibition of drug-induced apoptosis. Biochem Biophys Res Commun Apr. 7, 1998;245(1):266-71.

⁸⁶ Yen A, Roberson M S, Varvayanis S. Retinoic acid selectively activates the ERK2 but not JNK/SAPK or p38 MAP kinases when inducing myeloid differentiation. In vitro Cell Dev Biol Anim. 1999 October;35(9):527-32.

⁸⁷ Liu M K, Brownsey R W, Reiner N E. F interferon induces rapid and coordinate activation of mitogen-activated protein kinase (extracellular signal-regulated kinase) and calcium-independent protein kinase C in human monocytes. Infect Immun, July 1994, 2722-2731, Vol 62, No. 7.

⁸⁸ Nishiya T, Uehara T, Edamatsu H, Kaziro Y, Itoh H, Nomura Y. Activation of Stat1 and subsequent transcription of inducible nitric oxide synthase gene in C6 glioma cells is independent of interferon-γ-induced MAPK activation that is mediated by p21ras. FEBS Lett May 12, 1997;408(1):33-8.

⁸⁹ Lessor T, Yoo J Y, Davis M, Hamburger A W. Regulation of heregulin beta1-induced differentiation in a human breast carcinoma cell line by the extracellular-regulated kinase (ERK) pathway. J Cell Biochem Sep. 15, 1998;70(4):587-95.

⁹⁰ Marte B M, Graus-Porta D, Jeschke M, Fabbro D, Hynes N E, Taverna D. NDF/heregulin activates MAP kinase and p70/p85 S6 kinase during proliferation or differentiation of mammary epithelial cells. Oncogene Jan. 5, 1995;10(1):167-75.

⁹¹ Sepp-Lorenzino L, Eberhard I, Ma Z, Cho C, Serve H, Liu F, Rosen N, Lupu R. Signal transduction pathways induced by heregulin in MDA-MB-453 breast cancer cells. Oncogene Apr. 18, 1996;12(8):1679-87.

⁹² Fiddes R J, Janes P W, Sivertsen S P, Sutherland R L, Musgrove E A, Daly R J. Inhibition of the MAP kinase cascade blocks heregulin-induced cell cycle progression in T-47D human breast cancer cells. Oncogene May 28, 1998;16(21):2803-13.

⁹³ Park J A, Koh J Y. Induction of an immediate early gene egr-1 by zinc through extracellular signal-regulated kinase activation in cortical culture: its role in zinc-induced neuronal death. J. Neurochem. 1999 August;73(2):450-6.

⁹⁴ Kiss Z, Crilly K S, Tomono M. Bombesin and zinc enhance the synergistic mitogenic effects of insulin and phosphocholine by a MAP kinase-dependent mechanism in Swiss 3T3 cells. FEBS Lett. Sep. 22, 1997;415(1):71-4.

⁹⁵ Wu W, Graves L M, Jaspers 1, Devlin R B, Reed W, Samet J M. Activation of the EGF receptor signaling pathway in human airway epithelial cells exposed to metals. Am J. Physiol. 1999 November;277(5 Pt l):L924-31.

⁹⁶ Samet J M, Graves L M, Quay J, Dailey L A, Devlin R B, Ghio A J, Wu W, Bromberg P A, Reed W. Activation of MAPKs in human bronchial epithelial cells exposed to metals. Am J. Physiol. 1998 September;275(3 Pt l):L551-8.

⁹⁷ Migliaccio A, Di Domenico M, Castoria G, de Falco A, Bontempo P, Nola E, Auricchio F. Tyrosine kinase/p21ras/MAP-kinase pathway activation by estradiol-receptor complex in MCF-7 cells. EMBO J. Mar. 15, 1996;15(6):1292-300.

⁹⁸ Ruzycky A L. Effects of 17 beta-estradiol and progesterone on mitogen-activated protein kinase expression and activity in rat uterine smooth muscle. Eur J. Pharmacol. Apr. 11, 1996;300(3):247-54.

⁹⁹ Nuedling S, Kahlert S, Loebbert K, Meyer R, Vetter H, Grohe C. Differential effects of 17beta-estradiol on mitogen-activated protein kinase pathways in rat cardiomyocytes. FEBS Lett. Jul. 9, 1999;454(3):271-6.

¹⁰⁰ Laporte J D, Moore P E, Abraham J H, Maksym G N, Fabry B, Panettieri R A Jr, Shore S A. Role of ERK MAP kinases in responses of cultured human airway smooth muscle cells to IL-1beta. Am J. Physiol. 1999 November;277(5 Pt l):L943-51.

¹⁰¹ Larsen C M, Wadt K A, Juhl L F, Andersen H U, Karlsen A E, Su M S, Seedorf K, Shapiro L, Dinarello C A, Mandrup-Poulsen T. Interleukin-lbeta-induced rat pancreatic islet nitric oxide synthesis requires both the p38 and extracellular signal-regulated kinase ½ mitogen-activated protein kinases. J. Biol. Chem. Jun. 12, 1998;273(24):15294-300.

¹⁰² Daeipour M, Kumar G, Amaral M C, Nel A E. Recombinant IL-6 activates p42 and p44 mitogen-activated protein kinases in the IL-6 responsive B cell line, AF-10. J. Immunol. Jun. 1, 1993;150(11):4743-53.

¹⁰³ Leonard M, Ryan M P, Watson A J, Schramek H, Healy E. Role of MAP kinase pathways in mediating IL-6 production in human primary mesangial and proximal tubular cells. Kidney Int. 1999 October;56(4): 1366-77.

¹⁰⁴ Hartsough M T, Mulder K M. Transforming growth factor beta activation of p44mapk in proliferating cultures of epithelial cells. J. Biol. Chem. Mar. 31, 1995;270(13):7117-24.

¹⁰⁵ Yonekura A, Osaki M, Hirota Y, Tsukazaki T, Miyazaki Y, Matsumoto T, Ohtsuru A, Namba H, Shindo H, Yamashita S. Transforming growth factor-beta stimulates articular chondrocyte cell growth through p44/42 MAP kinase (ERK) activation. Endocr J. 1999 August;46(4):545-53.

¹⁰⁶ Strakova Z, Copland J A, Lolait S J, Soloff M S. ERK2 mediates oxytocin-stimulated PGE2 synthesis. Am J. Physiol. 1998 April;274(4Pt 1):E634-41.

¹⁰⁷ Copland J A, Jeng Y J, Strakova Z, Ives K L, Hellmich M R, Soloff M S. Demonstration of functional oxytocin receptors in human breast Hs578T cells and their up-regulation through a protein kinase C-dependent pathway. Endocrinology. 1999 May;140(5):2258-67.

¹⁰⁸ Subramanian C, Hasan S, Rowe M, Hottiger M, Orre R, Robertson E S. Epstein-Barr virus nuclear antigen 3C and prothymosin alpha interact with the p300 transcriptional coactivator at the CH1 and CH3/HAT domains and cooperate in regulation of transcription and histone acetylation. J. Virol. 2002 May;76(10):4699-708.

¹⁰⁹ Banas B, Eberle J, Banas B, Schlondorff D, Luckow B. Modulation of HIV-1 enhancer activity and virus production by cAMP. FEBS Lett. Dec. 7, 2001;509(2):207-12.

¹¹⁰ Deng L, de la Fuente C, Fu P, Wang L, Donnelly R, Wade J D, Lambert P, Li H, Lee C G, Kashanchi F. Acetylation of HIV-1 Tat by CBP/P300 increases transcription of integrated HIV-1 genome and enhances binding to core histones. Virology. Nov. 25, 2000;277(2):278-95.

¹¹¹ Cho S, Tian Y, Benjamin T L. Binding of p300/CBP co-activators by polyoma large T antigen. J. Biol. Chem. Sep. 7, 2001;276(36):33533-9.

¹¹² Wong H K, Ziff E B. Complementary functions of E1 conserved region I cooperate with conserved region 3 to activate adenovirus serotype 5 early promoters. J. Virol. 1994 August;68(8):4910-20.

¹¹³ Asano M, Murakami Y, Furukawa K, Yamaguchi-Iwai Y, Stake M, Ito Y. A Polayomavirus Enhancers Binding Protein, PEBP5, Responsive to 12-O-Tetradecanoylphorbol-13-Acetate but Distinct From AP-1. Journal of Virology 1990 64(12):5927-5938.

¹¹⁴ Higashino F, Yoshida K, Fujinaga Y, Kamio K, Fujinaga K. Isolation fo a cDNA Encoding the Adenovirus E1A Enhancer Binding Protein: A New Human Member of the ets Oncogene Family. Nucleic Acids Research 1993 21(3):547-553.

¹¹⁵ Laimins L A, Tsichlis P, Khoury G. Multiple Enhancer Domains in the 3′ Terminus of the Prague Strain of Rous Sarcoma Virus. Nucleic Acids Research 1984 12(16):6427-6442.

¹¹⁶ LaMarco K L, McKnight S L. Purification of a set of cellular polypeptides that bind to the purine-rich cis-regulatory element of herpes simplex virus immediate early genes. Genes Dev 1989 3(9): 1372-83.

¹¹⁷ Douville P, Hagmann M, Georgiev O, Schaffner W. Positive and negative regulation at the herpes simplex virus ICP4 and ICPO TAATGARAT motifs. Virology. Feb. 20, 1995;207(1): 107-16.

¹¹⁸ Boshart M, Weber F, Jahn G, Dorsch-Hasler K, Fleckenstein B, Schaffner W. A very strong enhancer is located upstream of an immediate early gene of human cytomegalovirus. Cell 1985 June;41(2):521-30.

¹¹⁹ Gunther C V, Graves B J. Identification of ETS domain proteins in murine T lymphocytes that interact with the Moloney murine leukemia virus enhancer. Mol Cell Biol 1994 14(11): 7569-80

¹²⁰ Flory E, Hoffmeyer A, Smola U, Rapp U R, Bruder J T. Raf-1 kinase targets GA-binding protein in transcriptional regulation of the human immunodeficiency virus type I promoter. J Virol 1996 April;70(4):2260-8.

¹²¹ Rawlins D R, Milman G, Hayward S D, Hayward G S. Sequence-specific DNA binding of the Epstein-Barr virus nuclear antigen (EBNA-1) to clustered sites in the plasmid maintenance region. Cell 1985 October;42(3):859-68.

¹²² Mauclere P, Mahieux R, Garcia-Calleja J M, SalIa R, Tekaia F, Millan J, De The G, Gessain A. A new HTLV-II subtype A isolate in an HIV-1 infected prostitute from Cameroon, Central Africa. AIDS Res Hum Retroviruses. 1995 August;11(8):989-93.

¹²³ Kornfeld H, Riedel N, Viglianti G A, Hirsch V, Mullins J I. Cloning of HTLV-4 and its relation to simian and human immunodeficiency viruses. Nature 1987 326(6113);610-613.

¹²⁴ Bruder J T, Hearing P. Cooperative binding of EF-IA to the E1A enhancer region mediates synergistic effects on E1A transcription during adenovirus infection. J. Virol. 1991 September;65(9):5084-7.

¹²⁵ Bruder J T, Hearing P. Nuclear factor EF-IA binds to the adenovirus E1A core enhancer element and to other transcriptional control regions. Mol Cell Biol. 1989 November;9(11):5143-53.

¹²⁶ Ostapchuk P, Diffley J F, Bruder J T, Stillman B, Levine A J, Hearing P. Interaction of a nuclear factor with the polyomavirus enhancer region. Proc Natl Acad Sci U S A. 1986 November;83(22):8550-4.

¹²⁷ Scholer H R, Gruss P. Specific interaction between enhancer-containing molecules and cellular components. Cell. 1984 February;36(2):403-11.

¹²⁸ Nuclear Respiratory Factor 2 should not be confused with NF-E2 Related Factor 2 which is also abbriviated NRF2 or NRF-2.

¹²⁹ Watanabe H, Imai T, Sharp P A, Handa H. Identification of two transcription factors that bind to specific elements in the promoter of the adenovirus early-region 4. Mol Cell Biol 1988 8(3): 1290-300. The transcription factor binds to the promoter of the adenovirus early-region 4 (E4). Hence the name E4 transcription factor 1.

¹³⁰ Enhancer Factor 1A should not be confused with Elongation Factor 1A which is also abbriviated EF-1A.

¹³¹ Suzuki F, Goto M, Sawa C, Ito S, Watanabe H, Sawada J, Handa H. Functional interactions of transcription factor human GA-binding protein subunits. J. Biol. Chem. Nov. 6, 1998;273(45):29302-8.

¹³² Sambrok J, MacCallum P, Russell D, eds. Molecular Cloning: A Laboratory Manual, 3rd Edition. Cold Spring Harbor Laboratory Press., 2001.

¹³³ Ausubel, et al., ets. Current Protocols in Molecular Biology. NY: John Wiley & Sons. 1998.

¹³⁴ Friedmann T, ed. The Development of Human Gene Therapy, Cold Spring Harbor Press, 1999.

¹³⁵ Jones P, Ramji D, Gacesa P, eds. Vectors: Expression Systems: Essential Techniques. (John Wiley and Sons, 1998

¹³⁶ Deshmukh, R. R., Cole, D. L. and Sanghvi, Y. S. Purification of Antisense Oligonucleotides in Methods in Enzymology, ed. M. Ian Phillips, v313, 1999, Academic Press, pp203-226.

¹³⁷ Daftary, G. S. and Taylor, H. S. Efficient liposome-mediated gene transfection and expression in the intact human uterus. Hum Gene Ther 12, 2121-2127 2001. Yale University School of Medicine, New Haven, Conn. 06520-8063, USA.

¹³⁸ Doherty, E. A and Doudna. Ribozyme structures and mechanisms. J. A. Ann. Rev. Biophys. Biomol Struct. 30, 457-475, 2001.

¹³⁹ Goodchild, J. Hammerhead ribozymes: biochemical and chemical considerations. Curr. Opin. Mol. Ther 2, 272-281, 2000. Review.

¹⁴⁰ Francois, J. -C., Lacoste, J., Lacroix, L. and J.-L. Mergny. Design of Antisense and Triplex-Forming Oligonucleotides. In Methods in Enzymology, ed. M Ian Phillips, v313, 1999, Academic Press, pp74-95

¹⁴¹ Hyrup, B and Nielsen, P. E. Peptide nucleic acids (PNA): synthesis, properties and potential applications. Bioorg. Med. Chem. 4: 5-23, 1996

¹⁴² Perry-O'Keefe, H., Yao, X. W, Coull, J. M., Fuchs, M. and Egholm, M. Peptide nucleic acid pre-gel hybridization: an alternative to Southern hybridization. Proc. Natl. Acad. Sci. USA 93: 14670-14675, 1996.

¹⁴³ Nielsen, P. E. (1999) Antisense Properties of Peptide Nucleic Acid. In Methods in Enzymology, ed. M. Ian Phillips, v313. 1999, Academic Press Academic Press, pp156-164.

¹⁴⁴ Harlow and Lane, eds. Using Antibodies: A Laboratory Manual. Cold Spring Harbor Press, 1999.

¹⁴⁵ Sambrook J, Fritsch E F, and Maniatis T. Molecular Cloning, Cold Spring Harbor Press, 1989.

¹⁴⁶ Kohler G, Milstein C. Continuous cultures of fused cells secreting antibody of predefined specificity. Nature 256: 495-497, 1975.

¹⁴⁷ Zola H. Monoclonal Antibodies: Preparation and Use of Monoclonal Antibodies and Engineered Antibody Derivatives (Basics: From Background to Bench), 2000.

¹⁴⁸ Ausubel F M, Brent R, Kingston R E, Moore D D, Seidman J G, Smith J A, Struhl K. Short protocols in molecular biology (4th ed.), John Wiley and Sons, Inc., 1999.

¹⁴⁹ Sapan C V, Lundblad R L, Price N C. Colorimetric protein assay techniques. Biotechnol Appl Biochem 29, 99-108, 1999.

¹⁵⁰ Manchester K L. Value of A260/A280 ratios for measurement of purity of nucleic acids. Biotechniques 19, 208-210, 1995.

¹⁵¹ Davis L G, Dibner M D, Battey J F. Basic methods in molecular biology, Elsevier Science Publishing Co., Inc. 1986.

¹⁵² Gizard F, Lavallee B, DeWitte F, Hum D W. A novel zinc finger protein TreP-132 interacts with CBP/p300 to regulate human p450scc gene expression. J. Biol. Chem. May 2001, in press.

¹⁵³ Heid C A, Stevens J, Livak K J, Williams P M. Real time quantitative PCR. Genome Res. 6, 986-994, 1996.

¹⁵⁴ Nuchprayoon I, Shang J, Simkevich C P, Luo M, Rosmarin A G, Friedman A D. An enhancer located between the neutrophil elastase and proteinase 3 promoters is activated by Sp] and an Ets Factor. J. Biol. Chem. 274, 1085-1091, 1999.

¹⁵⁵ Creighton, 1983, Proteins: Structures and Molecular Principles, W. H. Freeman & Co., NY, pp. 34-49

¹⁵⁶ Sanger F, Nicklen S, Coulson A R. DNA sequencing with chain-terminating inhibitors. Proc. Natl. Acad. Sci. USA 74, 5463-5467, 1977.

¹⁵⁷ Kristensen V N, Kelefiotis D, Kristensen T, Borresen-Dale A L. High-throughput methods for detection of genetic variation. Biotechniques 2001 February;30(2):318-22, 324,326 passim.

¹⁵⁸ Tawata M, Aida K, Onaya T. Screening for genetic mutations. A review. Comb Chem High Throughput Screen. 2000 February;3(1):1-9. Review.

¹⁵⁹ Pecheniuk N M, Walsh T P, Marsh N A. DNA technology for the detection of common genetic variants that predispose to thrombophilia. Blood Coagul Fibrinolysis 2000 December;11(8):683-700.

¹⁶⁰ Cotton R G. Current methods of mutation detection. Mutat Res. 1993 January;285(1):125-44. Review.

¹⁶¹ Prosser J. Detecting single-base mutations. Trends Biotechnol. 1993 June;11(6):238-46. Review.

¹⁶² Abrams E S, Murdaugh S E, Lerman L S. Comprehensive detection of single base changes in human genomic DNA using denaturing gradient gel electrophoresis and a GC clamp. Genomics. 1990 August;7(4):463-75.

¹⁶³ Forrest S, Cotton R G. Methods of detection of single base substitutions in clinical genetic practice. Mol Biol Med. 1990 October;7(5):451-9. Review.

¹⁶⁴ Graham C A, Hill A J. Introduction to DNA sequencing. Methods Mol. Biol. 2001;167:1-12. Review.

¹⁶⁵ Rapley R. eds. PCR sequencing protocols. Humana Press, Totowa, N.J., 1996.

¹⁶⁶ Marziali A, Akeson M. New DNA sequencing methods. Annu Rev Biomed Eng 2001;3:195-223.

¹⁶⁷ Dovichi N J, Zhang J. DNA sequencing by capillary array electrophoresis. Methods Mol. Biol. 2001;167:225-39. Review.

168 Huang G M. High-throughput DNA sequencing: a genomic data manufacturing process. DNA Seq. 1999;10(3): 149-53. Review.

¹⁶⁹ Schmalzing D, Koutny L, Salas-Solano O, Adourian A, Matsudaira P, Ehrlich D. Recent developments in DNA sequencing by capillary and microdevice electrophoresis. Electrophoresis. 1999 October;20(15-16):3066-77. Review.

170 Murray K K. DNA sequencing by mass spectrometry. J Mass Spectrom. 1996 November;31(11): 1203-15.

¹⁷¹ Cohen A S, Smisek D L, Wang B H. Emerging technologies for sequencing antisense oligonucleotides: capillary electrophoresis and mass spectrometry. Adv Chromatogr. 1996;36: 127-62. Review.

172 Griffin H G, Griffin A M. DNA sequencing. Recent innovations and future trends. Appl Biochem Biotechnol. 1993 January-February;38(1-2):147-59. Review.

173 Watts D, MacBeath J R. Automated fluorescent DNA sequencing on the ABI PRISM 310 Genetic Analyzer. Methods Mol. Biol. 2001;167:153-70. Review.

174 MacBeath J R, Harvey S S, Oldroyd N J. Automated fluorescent DNA sequencing on the ABI PRISM 377. Methods Mol. Biol. 2001;167:119-52. Review.

175 Smith L M, Brumley R L Jr, Buxton E C, Giddings M, Marchbanks M, Tong X. High-speed automated DNA sequencing in ultrathin slab gels. Methods Enzymol. 1996;271:219-37. Review.

176 Maxam A M, Gilbert W. A new method for sequencing DNA. Proc Natl Acad Sci U S A. 1977 February;74(2):560-4.

¹⁷⁷ Saleeba J A, Cotton R G. Chemical cleavage of mismatch to detect mutations. Methods Enzymol. 1993;217:286-95.

¹⁷⁸ Takahashi N, Hiyama K, Kodaira M, Satoh C. An improved method for the detection of genetic variations in DNA with denaturing gradient gel electrophoresis. Mutat Res. 1990 April;234(2):61-70.

¹⁷⁹ Cotton R G, Rodrigues N R, Campbell R D. Reactivity of cytosine and thymine in single-base-pair mismatches with hydroxylamine and osmium tetroxide and its application to the study of mutations. Proc Natl Acad Sci U S A. 1988 June;85(12):4397-401.

¹⁸⁰ Myers R M, Larin Z, Maniatis T. Detection of single base substitutions by ribonuclease cleavage at mismatches in RNA:DNA duplexes. Science. Dec. 13, 1985;230(4731):1242-6.

¹⁸¹ Myers R M, Lumelsky N, Lerman L S, Maniatis T. Detection of single base substitutions in total genomic DNA. Nature. Feb. 7-13, 1985;313(6002):495-8.

¹⁸² Xu J F, Yang Q P, Chen J Y, van Baalen M R, Hsu I C. Determining the site and nature of DNA mutations with the cloned MutY mismatch repair enzyme. Carcinogenesis. 1996 February;17(2):321-6.

¹⁸³ Hsu I C, Yang Q, Kahng M W, Xu J F. Detection of DNA point mutations with DNA mismatch repair enzymes. Carcinogenesis. 1994 August; 15(8):1657-62.

¹⁸⁴ Miterski B, Kruger R, Wintermeyer P, Epplen J T. PCR/SSCP detects reliably and efficiently DNA sequence variations in large scale screening projects. Comb Chem High Throughput Screen 2000 June;3(3):211-8.

¹⁸⁵ Jaeckel S, Epplen J T, Kauth M, Miterski B, Tschentscher F, Epplen C. Polymerase chain reaction-single strand conformation polymorphism or how to detect reliably and efficiently each sequence variation in many samples and many genes. Electrophoresis. 1998 December;19(18):3055-61. Review.

186 Hayashi K. PCR-SSCP: a method for detection of mutations. Genet Anal Tech Appl. 1992 June;9(3):73-9. Review.

187 Lipshutz R J, Morris D, Chee M, Hubbell E, Kozal M J, Shah N, Shen N, Yang R, Fodor S P. Using oligonucleotide probe arrays to access genetic diversity. Biotechniques 1995 September;19(3):442-7.

¹⁸⁸ Guo Z, Guilfoyle R A, Thiel A J, Wang R, Smith L M. Direct fluorescence analysis of genetic polymorphisms by hybridization with oligonucleotide arrays on glass supports. Nucleic Acids Res Dec. 11, 1994;22(24):5456-65.

¹⁸⁹ Saiki R K, Walsh P S, Levenson C H, Erlich H A. Genetic analysis of amplified DNA with immobilized sequence-specific oligonucleotide probes. Proc Natl Acad Sci U S A. 1989 August;86(16):6230-4.

¹⁹⁰ Efremov D G, Dimovski A J, Jankovic L, Efremov G D. Mutant oligonucleotide extension amplification: a nonlabeling polymerase-chain-reaction-based assay for the detection of point mutations. Acta Haematol 1991;85(2):66-70.

¹⁹¹ Gibbs R A, Nguyen P N, Caskey C T. Detection of single DNA base differences by competitive oligonucleotide priming. Nucleic Acids Res. Apr. 11, 1989;17(7):2437-48.

¹⁹² Geisler J P, Hatterman-Zogg M A, Rathe J A, Lallas T A, Kirby P, Buller R E. Ovarian cancer BRCA1 mutation detection: Protein truncation test (PTT) outperforms single strand conformation polymorphism analysis (SSCP). Hum Mutat. 2001 October;18(4):337-44.

¹⁹³ Moore W, Bogdarina 1, Patel U A, Perry M, Crane-Robinson C. Mutation detection in the breast cancer gene BRCA1 using the protein truncation test. Mol Biotechnol. 2000 February; 14(2):89-97.

¹⁹⁴ van der Luijt R, Khan P M, Vasen H, van Leeuwen C, Tops C, Roest P, den Dunnen J, Fodde R. Rapid detection of translation-terminating mutations at the adenomatous polyposis coli (APC) gene by direct protein truncation test. Genomics. Mar. 1, 1994;20(1):1-4.

¹⁹⁵ Roest P A, Roberts R G, Sugino S, van Ommen G J, den Dunnen J T. Protein truncation test (PTT) for rapid detection of translation-terminating mutations. Hum Mol Genet. 1993 October;2(10): 1719-21.

¹⁹⁶ Burnett W N. Western blotting electrophoretic transfer of proteins from SDS-polyacrylamide to unmodified nitrocellulose and autoradiographic detection with antibody and radioiodinated protein A. Ann Biochem, 112:195-203 1981.

¹⁹⁷ Virts E L, Raschke W C. The Role of Intron Sequences in High Level Expression from CD45 cDNA Constructs. J Biol Chem, 276, 19913-19920, 2001.

¹⁹⁸ Chen C, Okayama H. Calcium phosphate-mediated gene transfer: A highly efficient system for stably transforming cells with plasmid DNA. BioTech. 6, 632-638, 1988.

¹⁹⁹ Lopata M A, Cleveland D W, Sollner-Webb B. High-level expression of a chloramphenicol acetyltransferase gene by DEAE-dextran-mediated DNA transfection coupled with a dimethyl sulfoxide or glycerol shock treatment. Nucl. Acids Res. 12, 5707-5717, 1984.

²⁰⁰ Gorman C M, Moffat L F, Howard B H. Recombinant genomes which express chloramphenicol acetyltransferase in mammalian cells. Mol. Cell. Biol. 2, 1044-1051, 1982.

²⁰¹ Luo R Z, Peng H, Xu F, Bao J, Pang Y, Pershad R, Issa J J, Liao W S, Bast R C, Yu Y. Genomic structure and promoter characterization of an imprinted tumor suppressor gene ARHI(1). Biochim Biophys Acta, 1519, 216-222, 2001.

²⁰² Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. 57, 3145-3148, 1997.

²⁰³ Sucharov C, Basu A, Carter R S, Avadhani N G. A novel transcriptional initiator activity of the GABP factor binding ets sequence repeat from the murine cytochrome c oxidase Vb gene. Gene Expr. 5, 93-111, 1995.

²⁰⁴ Ouyang L, Jacob K K, Stanley F M. GABP mediates insulin-increased prolactin gene transcription. J. Biol. Chem. 271, 10425-10428, 1996.

²⁰⁵ Staskus K A, Embretson J E, Retzel E F, Beneke J, Haase A T. PCR in situ: new technologies with single cell resolution for the detection and investigation of viral latency and persistence. http://www.cbc.umn.edu/VirtLibrarv/Staskus/chap-shoot2.fm.html, 1994.

²⁰⁶ Schuurhuis G J, Muijen M M, Oberink J W, de Boer F, Ossenkoppele G J, Broxterman H J. Large populations of non-clonogenic early apoptotic CD34-positive cells are present in frozen-thawed peripheral blood stem cell transplants. Bone Marrow Transplant 27, 487-498, 2001.

²⁰⁷ Weyers A, Gorla N, Ugnia L, Garcia Ovando H, Chesta C. Increase of tissue lipid hydroperoxides as determination of oxidative stress. Biocell 25, 11-5 2001.

²⁰⁸ Brubacher J L, Bols N C. Chemically de-acetylated 2′,7′-dichlorodihydrofluorescein diacetate as a probe of respiratory burst activity in mononuclear phagocytes. J. Immunol. Tethods 25, 81-91, 2001.

²⁰⁹ Scholer H, Haslinger A, Heguy A, Holtgreve H, Karin M. In vivo Competition Between a Metallothionein Regulatory Element and the SV40 Enhancer. Science 1986 232: 76-80.

²¹⁰ Adam G1, Miller S J, Ulleras E, Franklin G C. Cell-type-specific modulation of PDGF-B regulatory elements via viral enhancer competition: a caveat for the use of reference plasmids in transient transfection assays. Gene. Oct. 31, 1996;178(1-2):25-9.

²¹¹ Allebach E S, Boettiger D, Pacifici M, Adams S L. Control of types I and II collagen and fibronectin gene expression in chondrocytes delineated by viral transformation. Mol Cell Biol. 1985 May;5(5):1002-8.

²¹² Parker I M, Smith A A, Gevers W. Absence of α₂(1) Procollagen Synthesis in a Clone of SV40-transformed WI-38 Human Fibroblasts. The Journal of Biological Chemistry, 1989, 264(13): 7147-7152.

²¹³ Moro L, Smith B D. Identification of collagen alpha1(1) trimer and normal type I collagen in a polyoma virus-induced mouse tumor. Arch Biochem Biophys 1977 July;182(1):33-41.

²¹⁴ Parker I M, Smith A A, Mundell K, Collins M, Boast S, Ramirez F. The abolition of collagen gene expression in SV40-transformed fibroblasts is associated with trans-acting factor switching. Nucleic Acids Research, 1992, 20(21): 5825-5830.

²¹⁵ Le Naour R, Lussiez C, Raoul H, Mabondzo A, Dormont D. Expression of cell adhesion molecules at the surface of in vitro human immunodeficiency virus type 1-infected human monocytes: relationships with tumor necrosis factor alpha, interleukin 1 beta, and interleukin 6 syntheses. AIDS Res Hum Retroviruses Jul. 1, 1997;13(10):841-55.

²¹⁶ Tanaka Y, Fukudome K, Hayashi M, Takagi S, Yoshie O. Induction of ICAM-1 and LFA-3 by Taxl of human T-cell leukemia virus type 1 and mechanism of down-regulation of ICAM-1 or LFA-1 in adult-T-cell-leukemia cell lines. Int J Cancer Feb. 8, 1995;60(4):554-61.

²¹⁷ Patarroyo M, Prieto J, Ernberg 1, Gahmberg C G. Absence, or low expression, of leukocyte adhesion molecules CD11 and CD18 on Burkitt lymphoma cells. Int J Cancer Jun. 15, 1988;41(6):901-7.

²¹⁸ Horvai A E, Xu L, Korzus E, Brard G, Kalafus D, Mullen T M, Rose D W, Rosenfeld M G, Glass C K. Nuclear integration of JAK/STAT and Ras/AP-1 signaling by CBP and p300. Proc Natl Acad Sci U S A. Feb. 18, 1997;94(4):1074-9.

²¹⁹ Pise-Masison C A, Mahieux R, Radonovich M, Jiang H, Brady J N. Human T-lymphotropic virus type I Tax protein utilizes distinct pathways for p53 inhibition that are cell type-dependent. J. Biol. Chem. Jan. 5, 2001;276(1):200-5.

²²⁰ Wang C, Fu M, D'Amico M, Albanese C, Zhou J N, Brownlee M, Lisanti M P, Chatterjee V K, Lazar M A, Pestell R G. Inhibition of cellular proliferation through IkappaB kinase-independent and peroxisome proliferator-activated receptor gamma-dependent repression of cyclin D1. Mol Cell Biol. 2001 May;21(9):3057-70.

²²¹ Ernst P, Wang J, Huang M, Goodman R H, Korsmeyer S J. MLL and CREB bind cooperatively to the nuclear coactivator CREB-binding protein. Mol Cell Biol. 2001 April;21(7):2249-58.

²²² Yuan W, Varga J. Transforming growth factor-beta repression of matrix metalloproteinase-1 in dermal fibroblasts involves Smad3. J. Biol. Chem. Oct. 19, 2001;276(42):38502-10.

²²³ Ghosh A K, Yuan W, Mori Y, Chen Sj, Varga J. Antagonistic regulation of type I collagen gene expression by interferon-gamma and transforming growth factor-beta. Integration at the level of p300/CBP transcriptional coactivators. J. Biol. Chem. Apr. 6, 2001;276(14):11041-8.

²²⁴ Li M, Pascual G, Glass C K. Peroxisome proliferator-activated receptor gamma-dependent repression of the inducible nitric oxide synthase gene. Mol Cell Biol. 2000 July;20(13):4699-707.

²²⁵ Nagarajan R P, Chen F, Li W, Vig E, Harrington M A, Nakshatri H, Chen Y. Repression of transforming-growth-factor-beta-mediated transcription by nuclear factor kappaB. Biochem J. Jun. 15, 2000;348 Pt 3:591-6.

²²⁶ Chen Y H, Ramos K S. A CCAAT/enhancer-binding protein site within antioxidant/electrophile response element along with CREB-binding protein participate in the negative regulation of rat GST-Ya gene in vascular smooth muscle cells. J. Biol. Chem. Sep. 1, 2000;275(35):27366-76.

²²⁷ Werner F, Jain M K, Feinberg M W, Sibinga N E, Pellacani A, Wiesel P, Chin M T, Topper J N, Perrella M A, Lee M E. Transforming growth factor-beta 1 inhibition of macrophage activation is mediated via Smad3. J. Biol. Chem. Nov. 24, 2000;275(47):36653-8.

²²⁸ Hipskind R A, Bilbe G. MAP kinase signaling cascades and gene expression in osteoblasts. Front Biosci. Aug. 1, 1998;3:D804-16.

²²⁹ Camps M, Nichols A, Arkinstall S. Dual specificity phosphatases: a gene family for control of MAP kinase function. FASEB J. 2000 January;14(1):6-16.

²³⁰ Saxena M, Mustelin T. Extracellular signals and scores of phosphatases: all roads lead to MAP kinase. Semin Immunol. 2000 August;12(4):387-96.

²³¹ Keyse S M. Protein phosphatases and the regulation of MAP kinase activity. Semin Cell Dev Biol. 1998 April;9(2):143-52.

²³² Schaeffer L, Duclert N, Huchet-Dymanus M, Changeux J P. Implication of a multisubunit Ets-related transcription factor in synaptic expression of the nicotinic acetylcholine receptor. EMBO J. Jun. 1, 1998;17(1l):3078-90.

²³³ Kumar A, Middleton A, Chambers T C, Mehta K D. Differential roles of extracellular signal-regulated kinase-½ and p38(MAPK) in interleukin-1 beta- and tumor necrosis factor-alpha-induced low density lipoprotein receptor expression in HepG2 cells. J. Biol. Chem. Jun. 19, 1998;273(25):15742-8.

²³⁴ Hoffmeyer A, Avots A, Flory E, Weber C K, Serfling E, Rapp U R. The GABP-responsive element of the interleukin-2 enhancer is regulated by JNK/SAPK-activating pathways in T lymphocytes. J. Biol. Chem. Apr. 24, 1998;273(17):10112-9.

²³⁵ Tomaras G D, Foster D A, Burrer C M, Taffet S M. ETS transcription factors regulate an enhancer activity in the third intron of TNF-alpha. J Leukoc Biol 1999 July;66(1):183-93.

²³⁶ Zhong H, Voll R E, Ghosh S. Phosphorylation of NF-kappa B p65 by PKA stimulates transcriptional activity by promoting a novel bivalent interaction with the coactivator CBP/p300. Mol Cell. 1998 April;1(5):661-71.

²³⁷ Bevilacqua M A, Faniello M C, Cimino F, Costanzo F. Okadaic acid stimulates H ferritin transcription in HeLa cells by increasing the interaction between the p300 CO-activator molecule and the transcription factor Bbf. Biochem Biophys Res Commun. Nov. 7, 1997;240(1):179-82.

²³⁸ Kuo M H, Allis C D. Roles of histone acetyltransferases and deacetylases in gene regulation. BioEassays 1998 20:615-626.

²³⁹ Hebbes T R, Clayton A L, Thorne A W, Crane-Robinson C. Core histone hyperacetylation co-maps with generalized DNase I sensitivity in the chicken beta-globin chromosomal domain. EMBO J. Apr. 15, 1994; 13(8): 1823-30.

²⁴⁰ Kuhnert P, Peterhans E, Pauli U. Chromatin structure and DNase I hypersensitivity in the transcriptionally active and inactive porcine tumor necrosis factor gene locus. Nucleic Acids Res. Apr. 25, 1992;20(8): 1943-8.

²⁴¹ Herschlag D., Johnson F. B., 1993. Synergism in transcriptional activation: a kinetic view. Genes and Development, 7:173-179.

²⁴² Eckner R, Ewen M E, Newsome D, Gerdes M, DeCaprio J A, Lawrence J B, Livingston D M. Molecular cloning and functional analysis of the adenovirus E1A-associated 300-kD protein (p300) reveals a protein with properties of a transcriptional adaptor. Genes Dev. Apr. 15, 1994;8(8):869-84.

²⁴³ Martin M E, Chinenov Y, Yu M, Schmidt T K, Yang X Y. Redox regulation of GA-binding protein-alpha DNA binding activity. J. Biol. Chem. Oct. 11, 1996;271(41):25617-23.

²⁴⁴ Bottinger E P, Shelley C S, Farokhzad O C, Arnaout M A. The human beta 2 integrin CD18 promoter consists of two inverted Ets cis elements. Mol Cell Biol. 1994 April;14(4):2604-15.

²⁴⁵ Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. Aug. 1, 1997;57(15):3145-8.

²⁴⁶ Atlas E, Stramwasser M, Whiskin K, Mueller C R. GA-binding protein alpha/beta is a critical regulator of the BRCA1 promoter. Oncogene Apr. 6, 2000;19(15):1933-40.

²⁴⁷ Li X R, Chong A S, Wu J, Roebuck K A, Kumar A, Parrillo J E, Rapp U R, Kimberly R P, Williams J W, Xu X. Transcriptional regulation of Fas gene expression by GA-binding protein and AP-1 in T cell antigen receptor.CD3 complex-stimulated T cells. J. Biol. Chem. Dec. 3, 1999;274(49):35203-10.

²⁴⁸ Donovan-Peluso M, George L D, Hassett A C. Lipopolysaccharide induction of tissue factor expression in THP-1 monocytic cells. Protein-DNA interactions with the promoter. J. Biol. Chem. Jan. 14, 1994;269(2): 1361-9.

²⁴⁹ Groupp E R, Donovan-Peluso M. Lipopolysaccharide induction of THP-1 cells activates binding of c-Jun, Ets, and Egr-1 to the tissue factor promoter. J. Biol. Chem. May 24, 1996;271(21):12423-30.

²⁵⁰ Hall A J, Vos H L, Bertina R M. Lipopolysaccharide induction of tissue factor in THP-1 cells involves Jun protein phosphorylation and nuclear factor kappaB nuclear translocation. J. Biol. Chem. Jan. 1, 1999;274(1):376-83.

²⁵¹ Holzmuller H, Moll T, Hofer-Warbinek R, Mechtcheriakova D, Binder B R, Hofer E. A transcriptional repressor of the tissue factor gene in endothelial cells. Arterioscler Thromb Vasc Biol. 1999 July;19(7):1804-11.

²⁵² Moll T, Czyz M, Holzmuller H, Hofer-Warbinek R, Wagner E, Winkler H, Bach F H, Hofer E. Regulation of the tissue factor promoter in endothelial cells. Binding of NF kappa B-, AP-1-, and Sp 1-like transcription factors. J. Biol. Chem. Feb. 24, 1995;270(8):3849-57.

²⁵³ Nathwani A C, Gale K M, Pemberton K D, Crossman D C, Tuddenham E G, McVey J H. Efficient gene transfer into human umbilical vein endothelial cells allows functional analysis of the human tissue factor gene promoter. Br J Haematol. 1994 September;88(1):122-8.

²⁵⁴ Mackman N, Fowler B J, Edgington T S, Morrissey J H. Functional analysis of the human tissue factor promoter and induction by serum. Proc Natl Acad Sci U S A. 1990 March;87(6):2254-8.

²⁵⁵ Key N S, Bach R R, Vercellotti G M, Moldow C F. Herpes simplex virus type I does not require productive infection to induce tissue factor in human umbilical vein endothelial cells. Lab Invest. 1993 June;68(6):645-51.

²⁵⁶ Oeth P, Yao J, Fan S T, Mackman N. Retinoic acid selectively inhibits lipopolysaccharide induction of tissue factor gene expression in human monocytes. Blood. Apr. 15, 1998;91(8):2857-65.

²⁵⁷ Pendurthi U R, Williams J T, Rao L V. Resveratrol, a polyphenolic compound found in wine, inhibits tissue factor expression in vascular cells: A possible mechanism for the cardiovascular benefits associated with moderate consumption of wine. Arterioscler Thromb Vasc Biol. 1999 February; 19(2):419-26.

²⁵⁸ Bach R R, Moldow C F. Mechanism of tissue factor activation on HL-60 cells. Blood. May 1, 1997;89(9):3270-6.

²⁵⁹ Nemerson Y, Giesen P L. Some thoughts about localization and expression of tissue factor. Blood Coagul Fibrinolysis. 1998 March;9 Suppl l:S45-7.

²⁶⁰ Wolberg A S, Kon R H, Monroe D M, Ezban M, Roberts H R, Hoffman M. Deencryption of Cellular Tissue Factor Is Independent of Its Cytoplasmic Domain. Biochem Biophys Res Commun. Jun. 7, 2000;272(2):332-336.

²⁶¹ Schecter A D, Giesen P L, Taby O, Rosenfield C L, Rossikhina M, Fyfe B S, Kohtz D S, Fallon J T, Nemerson Y, Taubman M B. Tissue factor expression in human arterial smooth muscle cells. TF is present in three cellular pools after growth factor stimulation. J Clin Invest. Nov. 1, 1997;100(9):2276-85.

²⁶² Pan J, Xia L, McEver R P. Comparison of promoters for the murine and human P-selectin genes suggests species-specific and conserved mechanisms for transcriptional regulation in endothelial cells. J. Biol. Chem. Apr. 17, 1998;273(16):10058-67.

²⁶³ Rosmarin A G, Caprio D G, Kirsch D G, Handa H, Simkevich C P. GABP and P U. I compete for binding, yet cooperate to increase CD18 (beta 2 leukocyte integrin) transcription. J Biol Chem Oct. 6, 1995;270(40):23627-33.

²⁶⁴ Rosen G D, Barks J L, lademarco M F, Fisher R J, Dean D C. An intricate arrangement of binding sites for the Ets family of transcription factors regulates activity of the alpha 4 integrin gene promoter. J. Biol. Chem. Jun. 3, 1994;269(22):15652-60.

²⁶⁵ Talmud P J, Palmen J, Walker M. Identification of genetic variation in the human hormone-sensitive lipase gene and 5′ sequences: homology of 5′ sequences with mouse promoter and identification of potential regulatory elements. Biochem Biophys Res Commun. Nov. 27, 1998;252(3):661-8.

²⁶⁶ Grober J, Laurell H, Blaise R, Fabry B, Schaak S, Holm C, Langin D. Characterization of the promoter of human adipocyte hormone-sensitive lipase. Biochem J. Dec. 1, 1997;328 (Pt 2):453-61.

²⁶⁷ Y U M, Yang X Y, Schmidt T, Chinenov Y, Wang R, Martin M E. GA-binding protein-dependent transcription initiator elements. Effect of helical spacing between polyomavirus enhancer a factor 3(PEA3)/Ets-binding sites on initiator activity. J. Biol. Chem. Nov. 14, 1997;272(46):29060-7.

²⁶⁸ Blaise R, Grober J, Rouet P, Tavernier G, Daegelen D, Langin D. Testis expression of hormone-sensitive lipase is conferred by a specific promoter that contains four regions binding testicular nuclear proteins. J. Biol. Chem. Apr. 2, 1999;274(14):9327-34.

²⁶⁹ Kawamura M, Jensen D F, Wancewicz E V, Joy L L, Khoo J C, Steinberg D. Hormone-sensitive lipase in differentiated 3T3-LI cells and its activation by cyclic AMP-dependent protein kinase. Proc Natl Acad Sci U S A. 1981 February;78(2):732-6.

²⁷⁰ Gordeladze J O, Hovik K E, Merendino J J, Hermouet S, Gutkind S, Accili D. Effect of activating and inactivating mutations of Gs- and G12-alpha protein subunits on growth and differentiation of 3T3-LI preadipocytes. J Cell Biochem. 1997 February;64(2):242-57.

²⁷¹ Awazu S, Nakata K, Hida D, Sakamoto T, Nagata K, Ishii N, Kanematsu T. Stable transfection of retinoblastoma gene promotes contact inhibition of cell growth and hepatocyte nuclear factor-1-mediated transcription in human hepatoma cells. Biochem Biophys Res Commun. Nov. 9, 1998;252(1):269-73.

²⁷² Armelin H A, Armelin M C, Kelly K, Stewart T, Leder P, Cochran B H, Stiles C D. Functional role for c-myc in mitogenic response to platelet-derived growth factor. Nature. Aug. 23-29, 1984;310(5979):655-60.

²⁷³ Cepko C L, Roberts B E, Mulligan R C. Construction and applications of a highly transmissible murine retrovirus shuttle vector. Cell. 1984 July;37(3):1053-62.

²⁷⁴ Higgins C, Chatterjee S, Cherington V. The block of adipocyte differentiation by a C-terminally truncated, but not by full-length, simian virus 40 large tumor antigen is dependent on an intact retinoblastoma susceptibility protein family binding domain. J. Virol. 1996 February;70(2):745-52.

²⁷⁵ Cherington V, Brown M, Paucha E, St Louis J, Spiegelman B M, Roberts T M. Separation of simian virus 40 large-T-antigen-transforming and origin-binding functions from the ability to block differentiation. Mol Cell Biol. 1988 March;8(3):1380-4.

²⁷⁶ Sellers W R, Kaelin W G Jr. Role of the retinoblastoma protein in the pathogenesis of human cancer. J Clin Oncol 1997 November; 15(11):3301-12.

²⁷⁷ Dou Q P, An B. R B and apoptotic cell death. Front Biosci. Apr. 6, 1998;3:d419-30.

²⁷⁸ Coppola J A, Lewis B A, Cole M D. Increased retinoblastoma gene expression is associated with late stages of differentiation in many different cell types. Oncogene 1990 November;5(11):1731-3.

²⁷⁹ Levine R A, Hopman T, Guo L, Chang M J, Johnson N. Induction of retinoblastoma gene expression during terminal growth arrest of a conditionally immortalized fetal rat lung epithelial cell line and during fetal lung maturation. Exp Cell Res. Mar. 15, 1998;239(2):264-76.

²⁸⁰ Slack R S, Hamel P A, Bladon T S, Gill R M, McBurney M W. Regulated expression of the retinoblastoma gene in differentiating embryonal carcinoma cells. Oncogene. 1993 June;8(6):1585-91.

²⁸¹ Richon V M, Rifkind R A, Marks P A. Expression and phosphorylation of the retinoblastoma protein during induced differentiation of murine erythroleukemia cells. Cell Growth Differ. 1992 July;3(7):413-20.

²⁸² Schwartz B, Avivi-Green C, Polak-Charcon S. Sodium butyrate induces retinoblastoma protein dephosphorylation, p 16 expression and growth arrest of colon cancer cells. Mol Cell Biochem. 1998 November; 188(1-2):21-30.

²⁸³ Thompson M E, Jensen R A, Oberniller P S, Page D L, Holt J T. Decreased expression of BRCA1 accelerates growth and is often present during sporadic breast cancer progression. Nat Genet 1995 April;9(4):444-50.

²⁸⁴ Rao V N, Shao N, Ahmad M, Reddy E S. Antisense RNA to the putative tumor suppressor gene BRCA1 transforms mouse fibroblasts. Oncogene. Feb. 1, 1996;12(3):523-8.

²⁸⁵ Holt J T, Thompson M E, Szabo C, Robinson-Benion C, Arteaga C L, King M C, Jensen R A. Growth retardation and tumour inhibition by BRCA1. Nat Genet. 1996 March; 12(3):298-302.

²⁸⁶ Tait D L, Obermiller P S, Redlin-Frazier S, Jensen R A, Welcsh P, Dann J, King M C, Johnson D H, Holt J T. A phase I trial of retroviral BRCA1sv gene therapy in ovarian cancer. Clin Cancer Res. 1997 November;3(11):1959-68.

²⁸⁷ Russell P A, Pharoah P D, De Foy K, Ramus S J, Symmonds I, Wilson A, Scott 1, Ponder B A, Gayther S A. Frequent loss of BRCA1 mRNA and protein expression in sporadic ovarian cancers. Int J Cancer. 2000 August;87(3):317-321.

²⁸⁸ R10 P G, Maurizis J C, Peffault de Latour M, Bignon Y J, Bernard-Gallon D J. Quantification of BRCA1 protein in sporadic breast carcinoma with or without loss of heterozygosity of the BRCA1 gene. Int J Cancer Mar. 15, 1999;80(6):823-6.

²⁸⁹ Rice J C, Massey-Brown K S, Futscher B W. Aberrant methylation of the BRCA1 CpG island promoter is associated with decreased BRCA1 mRNA in sporadic breast cancer cells. Oncogene. Oct. 8, 1998;17(14):1807-12.

²⁹⁰ Magdinier F, Ribieras S, Lenoir G M, Frappart L, Dante R. Down-regulation of BRCA1 in human sporadic breast cancer; analysis of DNA methylation patterns of the putative promoter region. Oncogene. Dec. 17, 1998;17(24):3169-76.

²⁹¹ Ozcelik H, To M D, Couture J, Bull S B, Andrulis I L. Preferential allelic expression can lead to reduced expression of BRCA1 in sporadic breast cancers. Int J Cancer. Jul. 3, 1998;77(1):1-6.

²⁹² Futreal P A, Liu Q, Shattuck-Eidens D, Cochran C, Harshman K, Tavtigian S, Bennett L M, Haugen-Strano A, Swensen J, Miki Y, et al. BRCA1 mutations in primary breast and ovarian carcinomas. Science Oct. 7, 1994;266(5182):120-2.

²⁹³ Merajver S D, Pham T M, Caduff R F, Chen M, Poy E L, Cooney K A, Weber B L, Collins F S, Johnston C, Frank T S. Somatic mutations in the BRCA1 gene in sporadic ovarian tumours. Nat Genet. 1995 April;9(4):439-43.

²⁹⁴ Catteau A, Harris W H, Xu C F, Solomon E. Methylation of the BRCA1 promoter region in sporadic breast and ovarian cancer: correlation with disease characteristics. Oncogene. Mar. 18, 1999; 18(11):1957-65.

²⁹⁵ Dobrovic A, Simpfendorfer D. Methylation of the BRCA1 gene in sporadic breast cancer. Cancer Res. Aug. 15, 1997;57(16):3347-50.

²⁹⁶ Davidson W F, Giese T, Fredrickson T N. Spontaneous development of plasmacytoid tumors in mice with defective Fas-Fas ligand interactions. J Exp Med. 1998 June;187(11):1825-38.

²⁹⁷ Drappa J, Vaishnaw A K, Sullivan K E, Chu J L, Elkon K B. Fas gene mutations in the Canale-Smith syndrome, an inherited lymphoproliferative disorder associated with autoimmunity. N Engl J. Med. Nov. 28, 1996;335(22): 1643-9.

²⁹⁸ Keane M M, Ettenberg S A, Lowrey G A, Russell E K, Lipkowitz S. Fas expression and function in normal and malignant breast cell lines. Cancer Res. Oct. 15, 1996;56(20):4791-8.

²⁹⁹ Gratas C, Tohma Y, Bamas C, Taniere P, Hainaut P, Ohgaki H. Up-regulation of Fas (APO-1/CD95) ligand and down-regulation of Fas expression in human esophageal cancer. Cancer Res. May 15, 1998;58(10):2057-62.

³⁰⁰ Strand S, Hofmann W J, Hug H, Muller M, Otto G, Strand D, Mariani S M, Stremmel W, Krammer P H, Galle P R. Lymphocyte apoptosis induced by CD95 (APO-1/Fas) ligand-expressing tumor cells—a mechanism of immune evasion? Nat Med. 1996 December;2(12):1361-6.

³⁰¹ Moller P, Koretz K, Leithauser F, Bruderlein S, Henne C, Quentmeier A, Krammer P H. Expression of APO-1 (CD95), a member of the NGF/TNF receptor superfamily, in normal and neoplastic colon epithelium. Int J Cancer. May 1, 1994;57(3):371-7.

³⁰² Leithauser F, Dhein J, Mechtersheimer G, Koretz K, Bruderlein S, Henne C, Schmidt A, Debatin K M, Krammer P H, Moller P. Constitutive and induced expression of APO-], a new member of the nerve growth factor/tumor necrosis factor receptor superfamily, in normal and neoplastic cells. Lab Invest. 1993 October;69(4):415-29.

³⁰³ Das H, Koizumi T, Sugimoto T, Chakraborty S, Ichimura T, Hasegawa K, Nishimura R. Quantitation of Fas and Fas ligand gene expression in human ovarian, cervical and endometrial carcinomas using real-time quantitative RT-PCR. Br J Cancer. 2000 May;82(10):1682-8.

³⁰⁴ Butler L M, Hewett P J, Butler W J, Cowled P A. Down-regulation of Fas gene expression in colon cancer is not a result of allelic loss or gene rearrangement. Br J Cancer. 1998 May;77(9):1454-9.

³⁰⁵ Bertoni F, Conconi A, Carobbio S, Realini C, Codegoni A M, Zucca E, Cavalli F. Analysis of Fas/CD95 gene somatic mutations in ovarian cancer cell lines. Int J Cancer. May 1, 2000;86(3):450.

³⁰⁶ Lee S H, Shin M S, Park W S, Kim S Y, Kim H S, Han J Y, Park G S, Dong S M, Pi J H, Kim C S, Kim S H, Lee J Y, Yoo N J. Alterations of Fas (Apo-1/CD95) gene in non-small cell lung cancer. Oncogene. Jun. 24, 1999; 18(25):3754-60.

³⁰⁷ Lee S H, Shin M S, Park W S, Kim S Y, Dong S M, Pi J H, Lee H K, Kim H S, Jang J J, Kim C S, Kim S H, Lee J Y, Yoo N J. Alterations of Fas (APO-1/CD95) gene in transitional cell carcinomas of urinary bladder. Cancer Res Jul. 1, 1999;59(13):3068-72.

³⁰⁸ Shin M S, Park W S, Kim S Y, Kim H S, Kang S J, Song K Y, Park J Y, Dong S M, Pi J H, Oh R R, Lee J Y, Yoo N J, Lee S H. Alterations of Fas (Apo-1/CD95) gene in cutaneous malignant melanoma. Am J Pathol 1999 June; 154(6):1785-91.

³⁰⁹ Butler L M, Dobrovic A, Bianco T, Cowled P A. Promoter region methylation does not account for the frequent loss of expression of the Fas gene in colorectal carcinoma. Br J Cancer. 2000 January;82(1):131-5.

³¹⁰ He H, Wang X, Gorospe M, Holbrook N J, Trush M A. Phorbol ester-induced mononuclear cell differentiation is blocked by the mitogen-activated protein kinase kinase (MEK) inhibitor PD98059. Cell Growth Differ 1999 May;10(5):307-15.

³¹¹ Vossen R C, Persoons M C, Slobbe-van Drunen M E, Bruggeman C A, van Dam-Mieras M C. Intracellular thiol redox status affects rat cytomegalovirus infection of vascular cells. Virus Res. 1997 May;48(2):173-83.

³¹² Scholz M, Cinatl J, Gross V, Vogel J U, Blaheta R A, Freisleben H J, Markus B H, Doerr H W. Impact of oxidative stress on human cytomegalovirus replication and on cytokine-mediated stimulation of endothelial cells. Transplantation. Jun. 27, 1996;61(12):1763-70.

³¹³ Ranjan D, Siquijor A, Johnston T D, Wu G, Nagabhuskahn M. The effect of curcumin on human B-cell immortalization by Epstein-Barr virus. Am Surg. 1998 January;64(1):47-51; discussion 51-2.

³¹⁴ Nakamura Y, Kawamoto N, Ohto Y, Torikai K, Murakami A, Ohigashi H. A diacetylenic spiroketal enol ether epoxide, AL-1, from Artemisia lactiflora inhibits 12-O-tetradecanoylphorbol-13-acetate-induced tumor promotion possibly by suppression of oxidative stress. Cancer Lett. Jun. 1, 1999;140(1-2):37-45.

³¹⁵ Allard J P, Aghdassi E, Chau J, Salit 1, Walmsley S. Oxidative stress and plasma antioxidant micronutrients in humans with HIV infection. Am J Clin Nutr 1998 January;67(1):143-7.

³¹⁶ Allard J P, Aghdassi E, Chau J, Tam C, Kovacs C M, Salit I E, Walmsley S L. Effects of vitamin E and C supplementation on oxidative stress and viral load in HIV-infected subjects. AIDS. Sep. 10, 1998;12(13):1653-9.

³¹⁷ Helen A, Krishnakumar K, Vijayammal P L, Augusti K T. Antioxidant effect of onion oil (Allium cepa. Linn) on the damages induced by nicotine in rats as compared to alpha-tocopherol. Toxicol Lett. Jul. 27, 2000;116(1-2):61-8.

³¹⁸ Yildiz D, Liu Y S, Ercal N, Armstrong D W. Comparison of pure nicotine- and smokeless tobacco extract-induced toxicities and oxidative stress. Arch Environ Contam Toxicol. 1999 November;37(4):434-9.

³¹⁹ Yildiz D, Ercal N, Armstrong D W. Nicotine enantiomers and oxidative stress. Toxicology Sep. 15, 1998;130(2-3):155-65.

³²⁰ Afaq F, Abidi P, Rahman Q. N-acetyl L-cysteine attenuates oxidant-mediated toxicity induced by chrysotile fibers. Toxicol Lett. Sep. 30, 2000;117(1-2):53-60.

³²¹ Abidi P, Afaq F, Arif J M, Lohani M, Rahman Q. Chrysotile-mediated imbalance in the glutathione redox system in the development of pulmonary injury. Toxicol Lett May 20, 1999; 106(1):31-9.

³²² Liu W, Ernst J D, Courtney Broaddus V. Phagocytosis of crocidolite asbestos induces oxidative stress, DNA damage, and apoptosis in mesothelial cells. Am J Respir Cell Mol. Biol. 2000 September;23(3):371-8.

³²³ Marczynski B, Kraus T, Rozynek P, Raithel H J, Baur X. Association between 8-hydroxy-2′-deoxyguanosine levels in DNA of workers highly exposed to asbestos and their clinical data, occupational and non-occupational confounding factors, and cancer. Mutat Res. Jul. 10, 2000;468(2):203-12.

³²⁴ Marczynski B, Rozynek P, Kraus T, Schlosser S, Raithel H J, Baur X. Levels of 8-hydroxy-2′-deoxyguanosine in DNA of white blood cells from workers highly exposed to asbestos in Germany. Mutat Res. Jul. 10, 2000;468(2):195-202.

³²⁵ Fisher C E, Rossi A G, Shaw J, Beswick P H, Donaldson K. Release of TNFalpha in response to SiC fibres: differential effects in rodent and human primary macrophages, and in macrophage-like cell lines. Toxicol In vitro. 2000 February; 14(1):25-31.

³²⁶ Brown D M, Beswick P H, Bell K S, Donaldson K. Depletion of glutathione and ascorbate in lung lining fluid by respirable fibres. Ann Occup Hyg. 2000 March;44(2):101-8.

³²⁷ Butel J S. Viral carcinogenesis: revelation of molecular mechanisms and etiology of human disease. Carcinogenesis. 2000 March;21(3):405-26.

³²⁸ zur Hausen H. Viruses in human cancers. Eur J Cancer. 1999 August;35(8):1174-81.

³²⁹ Hoppe-Seyler F, Butz K. Human tumor viruses. Anticancer Res. 1999 November-December;19(6A):4747-58.

³³⁰ Bonnet M, Guinebretiere J M, Kremmer E, Grunewald V, Benhamou E, Contesso G, Joab I. Detection of Epstein-Barr virus in invasive breast cancers. J Natl Cancer Inst. Aug. 18, 1999;91(16):1376-81.

³³¹ Labrecque L G, Barnes D M, Fentiman I S, Griffin B E. Epstein-Barr virus in epithelial cell tumors: a breast cancer study. Cancer Res. Jan. 1, 1995;55(1):39-45.

³³² Magrath I, Bhatia K. Breast cancer: a new Epstein-Barr virus-associated disease? J Natl Cancer Inst. Aug. 18, 1999;91(16):1349-50.

³³³ Rakowicz-Szulczynska E M, Jackson B, Szulczynska A M, Smith M. Human immunodeficiency virus type 1-like DNA sequences and immunoreactive viral particles with unique association with breast cancer. Clin Diagn Lab Immunol. 1998 September;5(5):645-53.

³³⁴ Sanserson C M, Smith G L. Cell motility and cell morphology: how some viruses take control. May 4, 1999, http://www-ermm.cbcu.cam.ac.uk/99000629 h.htm

³³⁵ Dore M, Korthuis R J, Granger D N, Entman M L, Smith C W. P-selectin mediates spontaneous leukocyte rolling in vivo. Blood Aug. 15, 1993;82(4):1308-16.

³³⁶ Shang X Z, Issekutz A C. Contribution of CD11a/CD18, CD11b/CD18, ICAM-1 (CD54) and -2 (CD102) to human monocyte migration through endothelium and connective tissue fibroblast barriers. Eur J. Immunol. 1998 June;28(6):1970-9.

³³⁷ Shang X Z, Lang B J, Issekutz A C. Adhesion molecule mechanisms mediating monocyte migration through synovial fibroblast and endothelium barriers: role for CD11/CD18, very late antigen-4 (CD49d/CD29), very late antigen-5 (CD49e/CD29), and vascular cell adhesion molecule-1 (CD106). J. Immunol. Jan. 1, 1998;160(1):467-74.

³³⁸ Meerschaert J, Furie M B. The adhesion molecules used by monocytes for migration across endothelium include CD11a/CD18, CD11b/CD18, and VLA-4 on monocytes and ICAM-1, VCAM-1, and other ligands on endothelium. J. Immunol. Apr. 15, 1995;154(8):4099-112.

³³⁹ Meerschaert J, Furie M B. Monocytes use either CD11/CD18 or VLA-4 to migrate across human endothelium in vitro. J. Immunol. Feb. 15, 1994;152(4):1915-26.

³⁴⁰ Chuluyan H E, Issekutz A C. VLA-4 integrin can mediate CD11/CD18-independent transendothelial migration of human monocytes. J Clin Invest. 1993 December;92(6):2768-77.

³⁴¹ Kavanaugh A F, Lightfoot E, Lipsky P E, Oppenheimer-Marks N. Role of CD11/CD18 in adhesion and transendothelial migration of T cells. Analysis utilizing CD18-deficient T cell clones. J. Immunol. Jun. 15, 1991;146(12):4149-56.

³⁴² Wu C, Fields A J, Kapteijn B A, McDonald J A. The role of alpha 4 beta 1 integrin in cell motility and fibronectin matrix assembly. J. Cell Sci. 1995 February;108 (Pt 2):821-9.

³⁴³ Saltzman W M, Livingston T L, Parkhurst M R. Antibodies to CD18 influence neutrophil migration through extracellular matrix. J Leukoc Biol. 1999 March;65(3):356-63.

³⁴⁴ Fernandez-Segura E, Garcia J M, Campos A. Topographic distribution of CD18 integrin on human neutrophils as related to shape changes and movement induced by chemotactic peptide and phorbol esters. Cell Immunol. Jul. 10, 1996;171(1):120-5.

³⁴⁵ Werr J, Xie X, Hedqvist P, Ruoslahti E, Lindbom L. beta1 integrins are critically involved in neutrophil locomotion in extravascular tissue In vivo. J Exp Med. Jun. 15, 1998;187(12):2091-6.

³⁴⁶ Ott 1, Fischer E G, Miyagi Y, Mueller B M, Ruf W. A role for tissue factor in cell adhesion and migration mediated by interaction with actin-binding protein 280. J Cell Biol. Mar. 9, 1998;140(5):1241-53.

³⁴⁷ Muller M, Albrecht S, Golfert F, Hofer A, Funk R H, Magdolen V, Flossel C, Luther T. Localization of tissue factor in actin-filament-rich membrane areas of epithelial cells. Exp Cell Res. Apr. 10, 1999;248(1): 136-47.

³⁴⁸ Cunningham C C, Gorlin J B, Kwiatkowski D J, Hartwig J H, Janmey P A, Byers H R, Stossel T P. Actin-binding protein requirement for cortical stability and efficient locomotion. Science. Jan. 17, 1992;255(5042):325-7.

³⁴⁹ Randolph G J, Luther T, Albrecht S, Magdolen V, Muller W A. Role of tissue factor in adhesion of mononuclear phagocytes to and trafficking through endothelium in vitro. Blood. Dec. 1, 1998;92(11):4167-77.

³⁵⁰ Chang L C, Wang J P. Examination of the signal transduction pathways leading to activation of extracellular signal-regulated kinase by formyl-methionyl-leucyl-phenylalanine in rat neutrophils. FEBS Lett. Jul. 2, 1999;454(1-2):165-8.

³⁵¹ Yagisawa M, Saeki K, Okuma E, Kitamura T, Kitagawa S, Hirai H, Yazaki Y, Takaku F, Yuo A. Signal transduction pathways in normal human monocytes stimulated by cytokines and mediators: comparative study with normal human neutrophils or transformed cells and the putative roles in functionality and cell biology. Exp Hematol. 1999 June;27(6):1063-76.

³⁵² Coffer P J, Geijsen N, M'rabet L, Schweizer R C, Maikoe T, Raaijmakers J A, Lammers J W, Koenderman L. Comparison of the roles of mitogen-activated protein kinase kinase and phosphatidylinositol 3-kinase signal transduction in neutrophil effector function. Biochem J Jan. 1, 1998;329 (Pt 1): 121-30.

³⁵³ Yamada T, Fan J, Shimokama T, Tokunaga O, Watanabe T. Induction of fatty streak-like lesions in vitro using a culture model system simulating arterial intima. Am J Pathol. 1992 December;141(6):1435-44.

³⁵⁴ Kusuhara M, Chait A, Cader A, Berk B C. Oxidized LDL stimulates mitogen-activated protein kinases in smooth muscle cells and macrophages. Arterioscler Thromb Vasc Biol. 1997 January; 17(1): 141-8.

³⁵⁵ Deigner H P, Claus R. Stimulation of mitogen activated protein kinase by LDL and oxLDL in human U-937 macrophage-like cells. FEBS Lett. May 6, 1996;385(3):149-53.

³⁵⁶ Balagopalakrishna C, Bhunia A K, Rifkind J M, Chatterjee S. Minimally modified low density lipoproteins induce aortic smooth muscle cell proliferation via the activation of mitogen activated protein kinase. Mol Cell Biochem. 1997 May;170(1-2):85-9.

³⁵⁷ Kamanna V S, Bassa B V, Vaziri N D, Roh D D. Atherogenic lipoproteins and tyrosine kinase mitogenic signaling in mesangial cells. Kidney Int Suppl 1999 July;71:S70-5.

³⁵⁸ Bassa B V, Roh D D, Kirschenbaum M A, Kamanna VSAtherogenic lipoproteins stimulate mesangial cell p42 mitogen-activated protein kinase. J Am Soc Nephrol. 1998 March;9(3):488-96.

³⁵⁹ Quinn M T, Parthasarathy S, Fong L G, Steinberg D. Oxidatively modified low density lipoproteins: a potential role in recruitment and retention of monocyte/macrophages during atherogenesis. Proc Natl Acad Sci U S A. 1987 May;84(9):2995-8.

³⁶⁰ Crutchley D J, Que B G. Copper-induced tissue factor expression in human monocytic THP-1 cells and its inhibition by antioxidants. Circulation. Jul. 15, 1995;92(2):238-43.

³⁶¹ Caspar-Bauguil S, Tkaczuk J, Haure M J, Durand M, Alcouffe J, Thomsen M, Salvayre R, Benoist H. Mildly oxidized low-density lipoproteins decrease early production of interleukin 2 and nuclear factor kappaB binding to DNA in activated T-lymphocytes. Biochem J. Jan. 15, 1999;337 (Pt 2):269-74.

³⁶² Matsumura T, Sakai M, Matsuda K, Furukawa N, Kaneko K, Shichiri M. C is-acting DNA elements of mouse granulocyte/macrophage colony-stimulating factor gene responsive to oxidized low density lipoprotein. J. Biol. Chem. Dec. 31, 1999;274(53):37665-72.

³⁶³ Hamilton T A, Major J A, Armstrong D, Tebo J M. Oxidized LDL modulates activation of NFkappaB in mononuclear phagocytes by altering the degradation if IkappaBs. J Leukoc Biol. 1998 November;64(5):667-74.

³⁶⁴ Schackelford R E, Misra U K, Florine-Casteel K, Thai S F, Pizzo S V, Adams D O. Oxidized low density lipoprotein suppresses activation of NF kappa B in macrophages via a pertussis toxin-sensitive signaling mechanism. J. Biol. Chem. Feb. 24, 1995;270(8):3475-8.

³⁶⁵ Ohlsson B G, Englund M C, Karlsson A L, Knutsen E, Erixon C, Skribeck H, Liu Y, Bondjers G, Wiklund O. Oxidized low density lipoprotein inhibits lipopolysaccharide-induced binding of nuclear factor-kappaB to DNA and the subsequent expression of tumor necrosis factor-alpha and interleukin-lbeta in macrophages. J Clin Invest Jul. 1, 1996;98(1):78-89.

³⁶⁶ Yan S D, Schmidt A M, Anderson G M, Zhang J, Brett J, Zou Y S, Pinsky D, Stern D. Enhanced cellular oxidant stress by the interaction of advanced glycation end products with their receptors/binding proteins. J. Biol. Chem. Apr. 1, 1994;269(13):9889-97.

³⁶⁷ Khechai F, Ollivier V, Bridey F, Amar M, Hakim J, de Prost D. Effect of advanced glycation end product-modified albumin on tissue factor expression by monocytes. Role of oxidant stress and protein tyrosine kinase activation. Arterioscler Thromb Vasc Biol. 1997 November;17(11):2885-90.

³⁶⁸ Brisseau G F, Dackiw A P, Cheung P Y, Christie N, Rotstein O D. Posttranscriptional regulation of macrophage tissue factor expression by antioxidants. Blood Feb. 15, 1995;85(4):1025-35.

³⁶⁹ Ichikawa K, Yoshinari M, Iwase M, Wakisaka M, Doi Y, Ino K, Yamamoto M, Fujishima M. Advanced glycosylation end products induced tissue factor expression in human monocyte-like U937 cells and increased tissue factor expression in monocytes from diabetic patients. Atherosclerosis. 1998 February; 136(2):281-7.

³⁷⁰ Carson S D, Pirruccello S J. Immunofluorescent studies of tissue factor on U87MG cells: evidence for non-uniform distribution. Blood Coagul Fibrinolysis. 1993 December;4(6):911-20.

³⁷¹ Lewis J C, Bennett-Cain A L, DeMars C S, Doellgast G J, Grant K W, Jones N L, Gupta M. Procoagulant activity after exposure of monocyte-derived macrophages to minimally oxidized low density lipoprotein. Co-localization of tissue factor antigen and nascent fibrin fibers at the cell surface. Am J Pathol. 1995 October; 147(4): 1029-40.

¹⁷² Cui M Z, Penn M S, Chisolm G M. Natlve and oxidized low density lipoprotein induction of tissue factor gene expression in smooth muscle cells is mediated by both Egr-1 and Sp1. J. Biol. Chem. Nov. 12, 1999;274(46):32795-802.

³⁷³ Fei H, Berliner J A, Parhami F, Drake T A. Regulation of endothelial cell tissue factor expression by minimally oxidized LDL and lipopolysaccharide. Arterioscler Thromb. 1993 November; 13(11):1711-7.

³⁷⁴ Li D, Mehta J L. Upregulation of endothelial receptor for oxidized LDL (LOX-1) by oxidized LDL and implications in apoptosis of human coronary artery endothelial cells: evidence from use of antisense LOX-1 mRNA and chemical inhibitors. Arterioscler Thromb Vasc Biol. 2000 April;20(4): 1116-22.

³⁷⁵ Maziere C, Auclair M, Djavaheri-Mergny M, Packer L, Maziere J C. Oxidized low density lipoprotein induces activation of the transcription factor NF kappa B in fibroblasts, endothelial and smooth muscle cells. Biochem Mol Biol Int. 1996 August;39(6):1201-7.

³⁷⁶ Montoya M C, Luscinskas F W, del Pozo M A, Aragones J, de Landazuri M O. Reduced intracellular oxidative metabolism promotes firm adhesion of human polymorphonuclear leukocytes to vascular endothelium under flow conditions. Eur J. Immunol. 1997 August;27(8):1942-51.

³⁷⁷ Therond P, Abella A, Laurent D, Couturier M, Chalas J, Legrand A, Lindenbaum A. In vitro study of the cytotoxicity of isolated oxidized lipid low-density lipoproteins fractions in human endothelial cells: relationship with the glutathione status and cell morphology. Free Radic Biol Med. Feb. 15, 2000;28(4):585-96.

³⁷⁸ Lizard G, Gueldry S, Sordet O, Monier S, Athias A, Miguet C, Bessede G, Lemaire S, Solary E, Gambert P. Glutathione is implied in the control of 7-ketocholesterol-induced apoptosis, which is associated with radical oxygen species production. FASEB J. 1998 December;12(15):1651-63.

³⁷⁹ Gray J L, Shankar R. Down regulation of CD11b and CD18 expression in atherosclerotic lesion-derived macrophages. Am Surg 1995 August;61(8):674-9; discussion 679-80.

³⁸⁰ Trach C C, Wulfroth P M, Severs N J, Robenek H. Influence of native and modified lipoproteins on migration of mouse peritoneal macrophages and the effect of the antioxidants vitamin E and Probucol. Eur J Cell Biol. 1996 October;71(2):199-205.

³⁸¹ Pataki M, Lusztig G, Robenek H. Endocytosis of oxidized LDL and reversibility of migration inhibition in macrophage-derived foam cells in vitro. A mechanism for atherosclerosis regression? Arterioscler Thromb. 1992 August;12(8):936-44.

³⁸² Quinn M T, Parthasarathy S, Steinberg D. Endothelial cell-derived chemotactic activity for mouse peritoneal macrophages and the effects of modified forms of low density lipoprotein. Proc Natl Acad Sci U S A. 1985 September;82(17):5949-53.

³⁸³ Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol. 2000 May;20(5):1262-75.

³⁸⁴ Pentikainen M O, Oorni K, Ala-Korpela M, Kovanen P T. Modified LDL—trigger of atherosclerosis and inflammation in the arterial intima. J Intern Med. 2000 March;247(3):359-70.

³⁸⁵ Nordestgaard B G, Hjelms E, Stender S, Kjeldsen K. Different efflux pathways for high and low density lipoproteins from porcine aortic intima. Arteriosclerosis. 1990 May-June;10(3):477-85.

³⁸⁶ Boren J, Olin K, Lee I, Chait A, Wight T N, Innerarity T L. Identification of the principal proteoglycan-binding site in LDL. A single-point mutation in apo-BI00 severely affects proteoglycan interaction without affecting LDL receptor binding. J Clin Invest. Jun. 15, 1998;101(12):2658-64.

³⁸⁷ Nordestgaard B G, Tybjaerg-Hansen A, Lewis B. Influx in vivo of low density, intermediate density, and very low density lipoproteins into aortic intimas of genetically hyperlipidemic rabbits. Roles of plasma concentrations, extent of aortic lesion, and lipoprotein particle size as determinants. Arterioscler Thromb. 1992 January;12(1):6-18.

³⁸⁸ Schwenke D C. Comparison of aorta and pulmonary artery: II. LDL transport and metabolism correlate with susceptibility to atherosclerosis. Circ Res. 1997 September;81(3):346-54.

³⁸⁹ Kao C H, Chen J K, Yang V C. Ultrastructure and permeability of endothelial cells in branched regions of rat arteries. Atherosclerosis. 1994 January;105(1):97-114.

³⁹⁰ Kao C H, Chen J K, Kuo J S, Yang V C. Visualization of the transport pathways of low density lipoproteins across the endothelial cells in the branched regions of rat arteries. Atherosclerosis. 1995 July;116(1):27-41.

³⁹¹ Nordestgaard B G, Wootton R, Lewis B. Selective retention of VLDL, IDL, and LDL in the arterial intima of genetically hyperlipidemic rabbits in vivo. Molecular size as a determinant of fractional loss from the intima-inner media. Arterioscler Thromb Vasc Biol 1995 April;15(4):534-42.

³⁹² Gerrity R G. The role of the monocyte in atherogenesis: II. Migration of foam cells from atherosclerotic lesions. Am J Pathol. 1981 May;103(2):191-200.

³⁹³ Faggiotto A, Ross R, Harker L. Studies of hypercholesterolemia in the nonhuman primate. I. Changes that lead to fatty streak formation. Arteriosclerosis. 1984 July-August;4(4):323-40.

³⁹⁴ Faggiotto A, Ross R. Studies of hypercholesterolemia in the nonhuman primate. II. Fatty streak conversion to fibrous plaque. Arteriosclerosis. 1984 July-August;4(4):341-56.

³⁹⁵ Kling D, Holzschuh T, Betz E. Recruitment and dynamics of leukocytes in the formation of arterial intimal thickening—a comparative study with normo- and hypercholesterolemic rabbits. Atherosclerosis. 1993 June;101 (1):79-96.

³⁹⁶ Landers S C, Gupta M, Lewis J C. Ultrastructural localization of tissue factor on monocyte-derived macrophages and macrophage foam cells associated with atherosclerotic lesions. Virchows Arch. 1994;425(1):49-54.

³⁹⁷ Malek A M, Alper S L, Izumo S. Hemodynamic shear stress and its role in atherosclerosis. JAMA. Dec. 1, 1999;282(21):2035-42.

³⁹⁸ Wissler R W, Vesselinovitch D. Can atherosclerotic plaques regress? Anatomic and biochemical evidence from nonhuman animal models. Am J Cardiol. Mar. 20, 1990;65(12):33F-40F.

³⁹⁹ Dudrick S J. Regression of atherosclerosis by the intravenous infusion of specific biochemical nutrient substrates in animals and humans. Ann Surg. 1987 September;206(3):296-315.

⁴⁰⁰ Tucker C F, Catsulis C, Strong J P, Eggen D A. Regression of early cholesterol-induced aortic lesions in rhesus monkeys. Am J Pathol. 1971 December;65(3):493-514.

⁴⁰¹ Collins R G, Velji R, Guevara N V, Hicks M J, Chan L, Beaudet A L. β-Selectin or intercellular adhesion molecule (ICAM)-1 deficiency substantially protects against atherosclerosis in apolipoprotein E-deficient mice. J Exp Med. Jan. 3, 2000; 191(1): 189-94.

⁴⁰² Dong Z M, Chapman S M, Brown A A, Frenette P S, Hynes R O, Wagner D D. The combined role of P- and E-selectins in atherosclerosis. J Clin Invest. Jul. 1, 1998;102(1):145-52.

⁴⁰³ Patel S S, Thiagarajan R, Willerson J T, Yeh E T. Inhibition of alpha4 integrin and ICAM-1 markedly attenuate macrophage homing to atherosclerotic plaques in ApoE-deficient mice. Circulation. Jan. 6-13, 1998;97(1):75-81.

⁴⁰⁴ Stary H C, Chandler A B, Glagov S, Guyton J R, Insull Jr W, Rosenfeld M E, Schaffer S A, Schwartz C J, Wagner W D, Wissler R W. A Definition of Initial, Fatty Streak, and Intermediate Lesions of Atherosclerosis. A Report From the Committee on Vascular Lesions of the Council on Arteriosclerosis, American Heart Association. AHA Medical/Scientific Statement. 1994. http://www.americanheart.org/Scientific/statements/1994/059401.html

⁴⁰⁵ Taylor-Wiedeman J, Sissons P, Sinclair J. Induction of endogenous human cytomegalovirus gene expression after differentiation of onocytes from healthy carriers. J. Virol. 1994 March;68(3): 1597-604.

⁴⁰⁶ Guetta E, Guetta V, Shibutani T, Epstein S E. Monocytes harboring cytomegalovirus: interactions with endothelial cells, smooth muscle cells, and oxidized low-density lipoprotein. Possible mechanisms for activating virus delivered by monocytes to sites of vascular injury. Circ Res 1997 July;81(1):8-16.

⁴⁰⁷ Ikuta K, Luftig R B. Inhibition of cleavage of Moloney murine leukemia virus gag and env coded precursor polyproteins by cerulenin. Virology. Oct. 15, 1986;154(1):195-206.

⁴⁰⁸ Katoh 1, Yoshinaka Y, Luftig R B. The effect of cerulenin on Moloney murine leukemia virus morphogenesis. Virus Res. 1986 August;5(2-3):265-76.

⁴⁰⁹ Goldfine H, Harley J B, Wyke J A. Effects of inhibitors of lipid synthesis on the replication of Rous sarcoma virus. A specific effect of cerulenin on the processing of major non-glycosylated viral structural proteins. Biochim Biophys Acta Sep. 22, 1978;512(2):229-40.

⁴¹⁰ Ibanez C E, Schrier R, Ghazal P, Wiley C, Nelson J A. Human cytomegalovirus productively infects primary differentiated macrophages. J. Virol. 1991 December;65(12):6581-8.

⁴¹¹ Lathey J L, Spector S A. Unrestricted replication of human cytomegalovirus in hydrocortisone-treated macrophages. J. Virol. 1991 November;65(11):6371-5.

⁴¹² Weinshenker B G, Wilton S, Rice G P. Phorbol ester-induced differentiation permits productive human cytomegalovirus infection in a monocytic cell line. J. Immunol. Mar. 1, 1988; 140(5): 1625-31.

⁴¹³ Gonczol E, Andrews P W, Plotkin S A. Cytomegalovirus replicates in differentiated but not in undifferentiated human embryonal carcinoma cells. Science. Apr. 13, 1984;224(4645):159-61.

⁴¹⁴ Guyton J R. The role of lipoproteins in atherogenesis. Adv Exp Med Biol. 1995;369:29-38.

⁴¹⁵ Stary H C, Chandler A B, Dinsmore R E, Fuster V, Glagov S, Insull W Jr, Rosenfeld M E, Schwartz C J, Wagner W D, Wissler R W. A Definition of Advanced Types of Atherosclerotic Lesions and a Histological Classification of Atherosclerosis: A Report From the Committee on Vascular Lesions of the Council on Arteriosclerosis, American Heart Association. AHA Medical/Scientific Statement. 1995. http://www.americanheart.org/Scientific/statements/1995/17950005.html

⁴¹⁶ Randolph G J, Furie M B. Mononuclear phagocytes egress from an in vitro model of the vascular wall by migrating across endothelium in the basal to apical direction: role of intercellular adhesion molecule 1 and the CD11/CD18 integrins. J Exp Med. Feb. 1, 1996;183(2):451-62.

⁴¹⁷ Zhou Y F, Guetta E, Yu Z X, Finkel T, Epstein S E. Human cytomegalovirus increases modified low density lipoprotein uptake and scavenger receptor mRNA expression in vascular smooth muscle cells. J Clin Invest Nov. 1, 1996;98(9):2129-38.

⁴¹⁸ Benditt E P, Barrett T, McDougall J K. Viruses in the etiology of atherosclerosis. Proc Natl Acad Sci U S A 1983 October;80(20):6386-9.

⁴¹⁹ Wang H, Liu B, Fu M, Zeng C. [Transcriptional expression of oncogenes and Rb antioncogene in experimental atherosclerotic lesions]. Hua Hsi I Ko Ta Hsueh Hsueh Pao. 1996 June;27(2):117-21. Chinese.

⁴²⁰ Claudio P P, Fratta L, Farina F, Howard C M, Stassi G, Numata S, Pacilio C, Davis A, Lavitrano M, Volpe M, Wilson J M, Trimarco B, Giordano A, Condorelli G. Adenoviral RB2/p130 gene transfer inhibits smooth muscle cell proliferation and prevents restenosis after angioplasty. Circ Res. Nov. 26, 1999;85(11): 1032-9.

⁴²¹ Schwartz L B, Moawad J, Svensson E C, Tufts R L, Meyerson S L, Baunoch D, Leiden J M. Adenoviral-mediated gene transfer of a constitutively active form of the retinoblastoma gene product attenuates neointimal thickening in experimental vein grafts. J Vasc Surg. 1999 May;29(5):874-81; discussion 882-3.

⁴²² Smith R C, Wills K N, Antelman D, Perlman H, Truong L N, Krasinski K, Walsh K. Adenoviral constructs encoding phosphorylation-competent full-length and truncated forms of the human retinoblastoma protein inhibit myocyte proliferation and neointima formation. Circulation. Sep. 16, 1997;96(6): 1899-905.

⁴²³ Gallo R, Padurean A, Jayaraman T, Marx S, Roque M, Adelman S, Chesebro J, Fallon J, Fuster V, Marks A, Badimon J J. Inhibition of intimal thickening after balloon angioplasty in porcine coronary arteries by targeting regulators of the cell cycle. Circulation. Apr. 27, 1999;99(16):2164-70.

⁴²⁴ Chiu B. Multiple infections in carotid atherosclerotic plaques. Am Heart J. 1999 November;138(5 Pt 2):534-536.

⁴²⁵ Nieto F J. Viruses and atherosclerosis: A critical review of the epidemiologic evidence. Am Heart J 1999 November;138(5 Pt 2):453-460.

⁴²⁶ Adam E, Melnick J L, Probtsfield J L, Petrie B L, Burek J, Bailey K R, McCollum C H, DeBakey M E. High levels of cytomegalovirus antibody in patients requiring vascular surgery for atherosclerosis. Lancet. Aug. 8, 1987;2(8554):291-3.

⁴²⁷ Li B, Xu C, Wang Q. The detection of the antibodies of human cytomegalovirus in the sera of patients with coronary heart disease. Chung Hua Nei Ko Tsa Chih. 1996 November;35(11):741-3. (in Chinese).

⁴²⁸ Liuzzo G, Caligiuri G, Grillo R L, et al. Helicobacter pylori and cytomegalovirus infectoins are strongly associated with atherosclerosis, but are not responsible for the instability of angina [abstract]. J Am Coll Cardiol 1997;29(suppl A):217A.

⁴²⁹ Blum A, Giladi M, Weinberg M, Kaplan G, Pasternack H, Laniado S, Miller H. High anti-cytomegalovirus (CMV) IgG antibody titer is associated with coronary artery disease and may predict post-coronary balloon angioplasty restenosis. Am J Cardiol. Apr. 1, 1998;81(7):866-8.

⁴³⁰ Sorlie P D, Nieto F J, Adam E, Folsom A R, Shahar E, Massing M. A prospective study of cytomegalovirus, herpes simplex virus 1, and coronary heart disease: the atherosclerosis risk in communities (ARIC) study. Arch Intern Med. Jul. 10, 2000;160(13):2027-32.

⁴³¹ Fabricant C G, Fabricant J. Atherosclerosis induced by infection with Marek's disease herpesvirus in chickens. Am Heart J 1999 November;138(5 Pt 2):S465-8.

⁴³² Dodet B, Plotkin S A. Infection and atherosclerosis. Am Heart J 1999 November; 138(5 Pt 2):417-418.

⁴³³ Fong I W. Emerging relations between infectious diseases and coronary artery disease and atherosclerosis. CMAJ. Jul. 11, 2000;163(1):49-56.

⁴³⁴ zur Hausen H. Viruses in human cancers. Eur J Cancer. 1999 December;35(14):1878-85.

⁴³⁵ Crawford L. Criteria for establishing that a virus is oncogenic. Ciba Found Symp. 1986; 120:104-16.

⁴³⁶ Sawada M, Miyake S, Ohdama S, Matsubara O, Masuda S, Yakumaru K, Yoshizawa Y. Expression of tissue factor in non-small-cell lung cancers and its relationship to metastasis. Br J Cancer. 1999 February;79(3-4):472-7.

⁴³⁷ Shigemori C, Wada H, Matsumoto K, Shiku H, Nakamura S, Suzuki H. Tissue factor expression and metastatic potential of colorectal cancer. Thromb Haemost. 1998 December;80(6):894-8.

⁴³⁸ Mueller B M, Reisfeld R A, Edgington T S, Ruf W. Expression of tissue factor by melanoma cells promotes efficient hematogenous metastasis. Proc Natl Acad Sci U S A. Dec. 15, 1992;89(24):11832-6.

⁴³⁹ Adamson A S, Francis J L, Witherow R O, Snell M E. Urinary tissue factor levels in prostatic carcinoma: a potential marker of metastatic spread? Br J Urol. 1993 May;71(5):587-92.

⁴⁴⁰ Kataoka H, Uchino H, Asada Y, Hatakeyama K, Nabeshima K, Sumiyoshi A, Koono M. Analysis of tissue factor and tissue factor pathway inhibitor expression in human colorectal carcinoma cell lines and metastatic sublines to the liver. Int J Cancer. Sep. 4, 1997;72(5):878-84.

⁴⁴¹ Sturm U, Luther T, Albrecht S, Flossel C, Grossmann H, Muller M. Immunohistological detection of tissue factor in normal and abnormal human mammary glands using monoclonal antibodies. Virchows Arch A Pathol Anat Histopathol. 1992;421(2):79-86.

⁴⁴² Hu T, Bach R R, Horton R, Konigsberg W H, Todd M B. Procoagulant activity in cancer cells is dependent on tissue factor expression. Oncol Res. 1994;6(7):321-7.

⁴⁴³ Ruf W, Fischer E G, Huang H Y, Miyagi Y, Ott I, Riewald M, Mueller B M. Diverse functions of protease receptor tissue factor in inflammation and metastasis. Immunol Res. 2000;21(2-3):289-92.

⁴⁴⁴ Schwartz J D, Simantov R. Thrombosis and malignancy: pathogenesis and prevention. In vivo. 1998 November-December; 12(6):619-24.

⁴⁴⁵ Bromberg M E, Konigsberg W H, Madison J F, Pawashe A, Garen A. Tissue factor promotes melanoma metastasis by a pathway independent of blood coagulation. Proc Natl Acad Sci U S A. Aug. 29, 1995;92(18):8205-9.

⁴⁴⁶ Gebken J, Luders B, Notbohm H, Klein H H, Brinckmann J, Muller P K, Batge B. Hypergravity stimulates collagen synthesis in human osteoblast-like cells: evidence for the involvement of p44/42 MAP-kinases (ERK1/2). J Biochem (Tokyo). 1999 October;126(4):676-82.

⁴⁴⁷ Byers P H, Duvic M, Atkinson M, Robinow M, Smith L T, Krane S M, Greally M T, Ludman M, Matalon R, Pauker S, Quanbeck D, Schwarze U. Ehlers-Danlos syndrome type VIIA and VIIB result from splice-junction mutations or genomic deletions that involve exon 6 in the COLI AI and COL I A2 genes of type I collagen. Am J Med Genet Oct. 3, 1997;72(1):94-105.

⁴⁴⁸ Giunta C, Superti-Furga A, Spranger S, Cole W G, Steinmann B. Ehlers-Danlos syndrome type VII: clinical features and molecular defects. J Bone Joint Surg Am 1999 February;81(2):225-38.

⁴⁴⁹ Grahame R. Joint hypermobility and genetic collagen disorders: are they related? Arch Dis Child, 1999, 80: 188-191.

⁴⁵⁰ Batti'e M C, Bigos S J, Sheehy A, Wortley M D. Spinal flexibility and individual factos that influence it. Physical Therapy 1987 67(5):653-658.

⁴⁵¹ Grahame R. How often, when and how does joint hypermobility lead to osteoarthritis? Br J Rheumatol 1989 August;28(4):320.

⁴⁵² Scott D, Bird H, Wright V. Joint laxity leading to osteoarthrosis. Rheumatol Rehabil. 1979 August; 18(3): 167-9.

⁴⁵³ Sharma L, Lou C, Felson D T, Dunlop D D, Kirwan-Mellis G, Hayes K W, Weinrach D, Buchanan T S. Laxity in healthy and osteoarthritic knees. Arthritis Rheum 1999 May;42(5):861-70.

⁴⁵⁴ Jonsson H, Valtysdottir S T, Kjartansson O, Brekkan A. Hypermobility associated with osteoarthritis of the thumb base: a clinical and radiological subset of hand osteoarthritis. Ann Rheum Dis 1996 August;55(8):540-3.

⁴⁵⁵ Cicuttini F M., Baker J R., Spector T D. The Association of Obesity with Osteoarthritis of the Hand and Knee in Women: A Twin Study. The Journal of Rheumatology, 1996, 23:7, 1221-1226.

⁴⁵⁶ Carman W J, Sowers M, Hawthorne V M, Weissfeld L A. Obesity as a risk factor for osteoarthritis of the hand and wrist: a prospective study. Am J Epidemiol Jan. 15, 1994; 139(2):119-29.

⁴⁵⁷ Van Saase JLCM, Vandenbroucke M P, van Romunde LKJ, Valkenburg H A. Osteoarthritis and Obesity in the General Population. A Relationship Calling for and Explanation. The Journal of Rheumatology, 1988, 15(7): 1152-1158.

⁴⁵⁸ Ferguson K A, Love L L, Ryan C F. Effect of mandibular and tongue protrusion on upper airway size during wakefulness. Am J Respir Crit Care Med 1997 May;155(5):1748-54.

⁴⁵⁹ Miyamoto K, Ozbek M M, Lowe A A, Sjoholm T T, Love L L, Fleetham J A, Ryan C F. Mandibular posture during sleep in patients with obstructive sleep apnoea. Arch Oral Biol 1999 August;44(8):657-64.

⁴⁶⁰ Mokdad A H, Serdula M K, Dietz W H, Bowman B A, Marks J S, Koplan J P. The spread of the obesity epidemic in the United States, 1991-1998. JAMA. Oct. 27, 1999;282(16):1519-22.

⁴⁶¹ Weinsier R L, Hunter G R, Heini A F, Goran M I, Sell S M. The etiology of obesity: relative contribution of metabolic factors, diet, and physical activity. Am J. Med. 1998 August; 105(2):145-50.

⁴⁶² Heini A F, Weinsier R L. Divergent trends in obesity and fat intake patterns: the American paradox. Am J. Med. 1997 March; 102(3):259-64.

⁴⁶³ Hebebrand J, Wulftange H, Goerg T, Ziegler A, Hinney A, Barth N, Mayer H, Remschmidt H. Epidemic obesity: are genetic factors involved via increased rates of assortative mating? Int J Obes Relat Metab Disord. 2000 March;24(3):345-53.

⁴⁶⁴ Hill J O, Peters J C. Environmental contributions to the obesity epidemic. Science. May 29, 1998;280(5368): 1371-4.

⁴⁶⁵ Koplan J P, Dietz W H. Caloric imbalance and public health policy. JAMA. Oct. 27, 1999;282(16):1579-81.

⁴⁶⁶ Beattie J H, Wood A M, Newman A M, Bremner 1, Choo KHA, Michalska A E, Duncan J S, Trayhurn P. Obesity and hyperleptinemia in metallothionein (-I and -II) null mice. Proc. Natl. Acad. Sci. USA 1998 95(1): 358-363.

⁴⁶⁷ Dong Z M, Gutierrez-Ramos J C, Coxon A, Mayadas T N, Wagner D D. A new class of obesity genes encodes leukocyte adhesion receptors. Proc. Natl. Acad. Sci. USA 1997 (94):7526-7530.

⁴⁶⁸ Large V, Reynisdottir S, Langin D, Fredby K, Klannemark M, Holm C, Arner P. Decreased expression and function of adipocyte hormone-sensitive lipase in subcutaneous fat cells of obese subjects. J Lipid Res. 1999 November;40(11):2059-66.

⁴⁶⁹ Elizalde M, Ryden M, van Harmelen V, Eneroth P, Gyllenhammar H, Holm C, Ramel S, Olund A, Arner P, Andersson K. Expression of nitric oxide synthases in subcutaneous adipose tissue of nonobese and obese humans. J Lipid Res. 2000 August;41(8):1244-51.

⁴⁷⁰ Maudsley S, Pierce K L, Zamah A M, Miller W E, Ahn S, Daaka Y, Lefkowitz R J, Luttrell L M. The beta(2)-adrenergic receptor mediates extracellular signal-regulated kinase activation via assembly of a multi-receptor complex with the epidermal growth factor receptor. J. Biol. Chem. Mar. 31, 2000;275(13):9572-80.

⁴⁷¹ Pierce K L, Maudsley S, Daaka Y, Luttrell L M, Lefkowitz R J. Role of endocytosis in the activation of the extracellular signal-regulated kinase cascade by sequestering and nonsequestering G protein-coupled receptors. Proc Natl Acad Sci U S A. Feb. 15, 2000;97(4):1489-94.

⁴⁷² Elorza A, Sarnago S, Mayor F Jr. Agonist-dependent modulation of G protein-coupled receptor kinase 2 by mitogen-activated protein kinases. Mol Pharmacol. 2000 April;57(4):778-83.

⁴⁷³ Luttrell L M, Ferguson S S, Daaka Y, Miller W E, Maudsley S, Della Rocca G J, Lin F, Kawakatsu H, Owada K, Luttrell D K, Caron M G, Lefkowitz R J. Beta-arrestin-dependent formation of beta2 adrenergic receptor-Src protein kinase complexes. Science. Jan. 29, 1999;283(5402):655-61.

⁴⁷⁴ Daaka Y, Luttrell L M, Ahn S, Della Rocca G J, Ferguson S S, Caron M G, Lefkowitz R J. Essential role for G protein-coupled receptor endocytosis in the activation of mitogen-activated protein kinase. J. Biol. Chem. Jan. 9, 1998;273(2):685-8.

⁴⁷⁵ Cao W, Luttrell L M, Medvedev A V, Pierce K L, Daniel K W, Dixon T M, Lefkowitz R J, Collins S. Direct Binding of Activated c-Src to the Beta3-Adrenergic Receptor is Required for MAP Kinase Activation. J. Biol. Chem. Sep. 29, 2000.

⁴⁷⁶ Gerhardt C C, Gros J, Strosberg A D, Issad T. Stimulation of the extracellular signal-regulated kinase ½ pathway by human beta-3 adrenergic receptor: new pharmacological profile and mechanism of activation. Mol Pharmacol. 1999 February;55(2):255-62.

⁴⁷⁷ Soeder K J, Snedden S K, Cao W, Della Rocca G J, Daniel K W, Luttrell L M, Collins S. The beta3-adrenergic receptor activates mitogen-activated protein kinase in adipocytes through a Gi-dependent mechanism. J. Biol. Chem. Apr. 23, 1999;274(17): 12017-22.

⁴⁷⁸ Hellstrom L, Langin D, Reynisdottir S, Dauzats M, Arner P. Adipocyte lipolysis in normal weight subjects with obesity among first-degree relatives. Diabetologia. 1996 August;39(8):921-8.

⁴⁷⁹ Shimizu Y, Tanishita T, Minokoshi Y, Shimazu T. Activation of mitogen-activated protein kinase by norepinephrine in brown adipocytes from rats. Endocrinology. 1997 January;138(1):248-53.

⁴⁸⁰ Yarwood S J, Kilgour E, Anderson N G. Cyclic AMP stimulates the phosphorylation and activation of p42 and p44 mitogen-activated protein kinases in 3T3-F442A preadipocytes. Biochem Biophys Res Commun. Jul. 25, 1996;224(3):734-9.

⁴⁸¹ Gerhardt C C, Gros J, Strosberg A D, Issad T. Stimulation of the extracellular signal-regulated kinase ½ pathway by human beta-3 adrenergic receptor: new pharmacological profile and mechanism of activation. Mol Pharmacol. 1999 February;55(2):255-62.

⁴⁸² Hellstrom L, Reynisdottir S. Influence of heredity for obesity on adipocyte lipolysis in lean and obese subjects. Int J Obes Relat Metab Disord. 2000 March;24(3):340-4.

⁴⁸³ Bougneres P, Stunff C L, Pecqueur C, Pinglier E, Adnot P, Ricquier D. In vivo resistance of lipolysis to epinephrine. A new feature of childhood onset obesity. J Clin Invest. Jun. 1, 1997;99(11):2568-73.

⁴⁸⁴ Horowitz J F, Klein S. Whole body and abdominal lipolytic sensitivity to epinephrine is suppressed in upper body obese women. Am J Physiol Endocrinol Metab. 2000 June;278(6):E 1144-52.

⁴⁸⁵ Osuga J, Ishibashi S, Oka T, Yagyu H, Tozawa R, Fujimoto A, Shionoiri F, Yahagi N, Kraemer F B, Tsutsumi O, Yamada N. Targeted disruption of hormone-sensitive lipase results in male sterility and adipocyte hypertrophy, but not in obesity. Proc Natl Acad Sci U S A. Jan. 18, 2000;97(2):787-92.

⁴⁸⁶ Classon M, Kennedy B K, Mulloy R, Harlow E. Opposing roles of pRB and p107 in adipocyte differentiation. Proc Natl Acad Sci U S A. Sep. 26, 2000;97(20): 10826-31.

⁴⁸⁷ Puigserver P, Ribot J, Serra F, Gianotti M, Bonet M L, Nadal-Ginard B, Palou A. Involvement of the retinoblastoma protein in brown and white adipocyte cell differentiation: functional and physical association with the adipogenic transcription factor C/EBPalpha. Eur J Cell Biol. 1998 October;77(2): 117-23.

⁴⁸⁸ Roncari D A, Kindler S, Hollenberg C H. Excessive proliferation in culture of reverted adipocytes from massively obese persons. Metabolism. 1986 January;35(1):1-4.

⁴⁸⁹ Roncari D A, Lau D C, Kindler S. Exaggerated replication in culture of adipocyte precursors from massively obese persons. Metabolism. 1981 May;30(5):425-7.

⁴⁹⁰ Stock S, Granstrom L, Backman L, Matthiesen A S, Uvnas-Moberg K. Elevated plasma levels of oxytocin in obese subjects before and after gastric banding. Int J Obes. 1989; 13(2):213-22.

⁴⁹¹ Johnson S R, Kolberg B H, Varner M W, Railsback L D. Maternal obesity and pregnancy. Surg Gynecol Obstet. 1987 May; 164(5):431-7.

⁴⁹² Yakinci C, Pac A, Kucukbay F Z, Tayfun M, Gul A. Serum zinc, copper, and magnesium levels in obese children. Acta Paediatr Jpn. 1997 June;39(3):339-41.

⁴⁹³ D'Ocon C, Alonso de Armino V, Frasquet I. Levels of Zn and Cu in the serum of a diabetic population. Rev Esp Fisiol. 1987 September;43(3):335-8. Spanish.

⁴⁹⁴ Taneja S K, Mahajan M, Arya P. Excess bioavailability of zinc may cause obesity in humans. Experientia. Jan. 16, 1996;52(1):31-3.

⁴⁹⁵ Ludvik B, Nolan J J, Baloga J, Sacks D, Olefsky J. Effect of obesity on insulin resistance in normal subjects and patients with NIDDM. Diabetes. 1995 September;44(9):1121-5.

⁴⁴⁶ Prager R, Wallace P, Olefsky J M. Hyperinsulinemia does not compensate for peripheral insulin resistance in obesity. Diabetes. 1987 March;36(3):327-34.

⁴⁹⁷ Tasaka Y, Yanagisawa K, Iwamoto Y. Human plasma leptin in obese subjects and diabetics. Endocr J. 1997 October;44(5):671-6.

⁴⁹⁸ Bjorbaek C, El-Haschimi K, Frantz J D, Flier J S. The role of SOCS-3 in leptin signaling and leptin resistance. J. Biol. Chem. Oct. 15, 1999;274(42):30059-65.

⁴⁹⁹ de Waard F, Poortman J, de Pedro-Alvarez Ferrero M, Baanders-van Halewijn E A. Weight reduction and oestrogen excretion in obese post-menopausal women. Maturitas. 1982 August;4(2):155-62.

⁵⁰⁰ Cauley J A, Gutai J P, Kuller L H, LeDonne D, Powell J G. The epidemiology of serum sex hormones in pₒstmenopausal women. Am J Epidemiol. 1989 June; 129(6): 1120-31.

⁵⁰¹ Cauley J A, Gutai J P, Kuller L H, Scott J, Nevitt M C. Black-white differences in serum sex hormones and bone mineral density. Am J Epidemiol. May 15, 1994;139(10):1035-46.

⁵⁰² Galea J, Armstrong J, Gadsdon P, Holden H, Francis S E, Holt C M. Interleukin-1 beta in coronary arteries of patients with ischemic heart disease. Arterioscier Thromb Vasc Biol. 1996 August; 16(8): 1000-6.

⁵⁰³ Hasdai D, Scheinowitz M, Leibovitz E, Sclarovsky S, Eldar M, Barak V. Increased serum concentrations of interleukin-1 beta in patients with coronary artery disease. Heart. 1996 July;76(1):24-8.

⁵⁰⁴ Pickup J C, Mattock M B, Chusney G D, Burt D. NIDDM as a disease of the innate immune system: association of acute-phase reactants and interleukin-6 with metabolic syndrome X. Diabetologia. 1997 November;40(11):1286-92.

⁵⁰⁵ Pickup J C, Crook M A. Is type II diabetes mellitus a disease of the innate immune system? Diabetologia. 1998 October;41(10): 1241-8.

⁵⁰⁶ Hrnciar J, Gabor D, Hrnciarova M, Okapcova J, Szentivanyi M, Kurray P. [Relation between cytokines (TNF-alpha, IL-1 and 6) and homocysteine in android obesity and the phenomenon of insulin resistance syndromes]. Vnitr Lek. 1999 January;45(1):11-6. Slovak.

⁵⁰⁷ Klein J B, Rane M J, Scherzer J A, Coxon P Y, Kettritz R, Mathiesen J M, Buridi A, McLeish K R. Granulocyte-macrophage colony-stimulating factor delays neutrophil constitutive apoptosis through phosphoinositide 3-kinase and extracellular signal-regulated kinase pathways. J Immunol Apr. 15, 2000; 164(8):4286-91.

⁵⁰⁸ Valledor A F, Comalada M, Xaus J, Celada A. The differential time-course of extracellular-regulated kinase activity correlates with the macrophage response toward proliferation or activation. J Biol Chem Mar. 10, 2000;275(10):7403-9.

⁵⁰⁹ Thomas R S, Tymms M J, McKinlay L H, Shannon M F, Seth A, Kola I. ETS1, NFkappaB and AP1 synergistically transactivate the human GM-CSF promoter. Oncogene. Jun. 12, 1997;14(23):2845-55.

⁵¹⁰ Dhurandhar N V, Israel B A, Kolesar J M, Mayhew G F, Cook M E, Atkinson R L. Increased adiposity in animals due to a human virus. Int J Obes Relat Metab Disord. 2000 August;24(8):989-96.

⁵¹¹ Behrens G M, Stoll M, Schmidt R E. Lipodystrophy syndrome in HIV infection: what is it, what causes it and how can it be managed? Drug Saf. 2000 July;23(1):57-76.

⁵¹² Engelson E S, Kotler D P, Tan Y, Agin D, Wang J, Pierson R N Jr, Heymsfield S B. Fat distribution in HIV-infected patients reporting truncal enlargement quantified by whole-body magnetic resonance imaging. Am J Clin Nutr. 1999 June;69(6):1162-9.

⁵¹³ Clement K, Vaisse C, Lahlou N, Cabrol S, Pelloux V, Cassuto D, Gourmelen M, Dina C, Chambaz J, Lacorte J M, Basdevant A, Bougneres P, Lebouc Y, Froguel P, Guy-Grand B. A mutation in the human leptin receptor gene causes obesity and pituitary dysfunction. Nature. Mar. 26, 1998;392(6674):398-401.

⁵¹⁴ Chen H, Charlat O, Tartaglia L A, Woolf E A, Weng X, Ellis S J, Lakey N D, Culpepper J, Moore K J, Breitbart R E, Duyk G M, Tepper R1, Morgenstern J P. Evidence that the diabetes gene encodes the leptin receptor: identification of a mutation in the leptin receptor gene in db/db mice. Cell Feb. 9, 1996;84(3):491-5.

⁵¹⁵ da Silva B A, Bjorbaek C, Uotani S, Flier J S. Functional properties of leptin receptor isoforms containing the gln-->pro extracellular domain mutation of the fatty rat. Endocrinology. 1998 September; 139(9):3681-90.

⁵¹⁶ Yamashita T, Murakami T, Otani S, Kuwajima M, Shima K. Leptin receptor signal transduction: OBRa and OBRb of fa type. Biochem Biophys Res Commun. May 29, 1998;246(3):752-9.

⁵¹⁷ del Rey A, Besedovsky H. Antidiabetic effects of interleukin 1. Proc Natl Acad Sci U S A 1989 August;86(15):5943-7.

⁵¹⁸ Ilyin S E, Plata-Salaman C R. Molecular regulation of the brain interleukin-1 beta system in obese (fa/fa) and lean (Fa/Fa) Zucker rats. Brain Res Mol Brain Res. Dec. 31, 1996;43(1-2):209-18.

⁵¹⁹ Luheshi G N, Gardner J D, Rushforth D A, Loudon A S, Rothwell N J. Leptin actions on food intake and body temperature are mediated by IL-1. Proc Natl Acad Sci U S A Jun. 8, 1999;96(12):7047-52.

⁵²⁰ Plata-Salaman C R, Vasselli J R, Sonti G. Differential responsiveness of obese (fa/fa) and lean (Fa/Fa) Zucker rats to cytokine-induced anorexia. Obes Res 1997 January;5(1):36-42.

⁵²¹ Faggioni R, Fuller J, Moser A, Feingold K R, Grunfeld C. LPS-induced anorexia in leptin-deficient (ob/ob) and leptin receptor-deficient (db/db) mice. Am J. Physiol. 1997 July;273(I Pt 2):R181-6.

⁵²² Baroni M G, D'Andrea M P, Montali A, Pannitteri G, Barilla F, Campagna F, Mazzei E, Lovari S, Seccareccia F, Campa P P, Ricci G, Pozzilli P, Urbinati G, Arca M. A common mutation of the insulin receptor substrate-1 gene is a risk factor for coronary artery disease. Arterioscler Thromb Vasc Biol. 1999 December; 19(12):2975-80.

⁵²³ Park K, Kim S J, Bang Y J, Park J G, Kim N K, Roberts A B, Sporn M B. Genetic changes in the transforming growth factor beta (TGF-beta) type 1I receptor gene in human gastric cancer cells: correlation with sensitivity to growth inhibition by TGF-beta. Proc Natl Acad Sci U S A. Sep. 13, 1994;91(19):8772-6.

⁵²⁴ Myeroff L L, Parsons R, Kim S J, Hedrick L, Cho K R, Orth K, Mathis M, Kinzier K W, Lutterbaugh J, Park K, et al. A transforming growth factor beta receptor type II gene mutation common in colon and gastric but rare in endometrial cancers with microsatellite instability. Cancer Res. Dec. 1, 1995;55(23):5545-7.

⁵²⁵ McCaffrey T A, Du B, Consigli S, Szabo P, Bray P J, Hartner L, Weksler B B, Sanborn T A, Bergman G, Bush H L Jr. Genomic instability in the type II TGF-beta1 receptor gene in atherosclerotic and restenotic vascular cells. J Clin Invest. Nov. 1, 1997;100(9):2182-8.

⁵²⁶ Serra R, Johnson M, Filvaroff E H, LaBorde J, Sheehan D M, Derynck R, Moses H L. Expression of a truncated, kinase-defective TGF-beta type II receptor in mouse skeletal tissue promotes terminal chondrocyte differentiation and osteoarthritis. J Cell Biol. Oct. 20, 1997;139(2):541-52.

⁵²⁷ Caruso A, Fortini A, Fulghesu A M, Pistilli E, Cucinelli F, Lanzone A, Mancuso S. Ovarian sensitivity to follicle-stimulating hormone during the follicular phase of the human menstrual cycle and in patients with polycystic ovarian syndrome. Fertil Steril. 1993 January;59(I): 115-20.

⁵²⁸ Wronski T J, Schenck P A, Cintron M, Walsh C C. Effect of body weight on osteopenia in ovariectomized rats. Calcif Tissue Int. 1987 March;40(3):155-9.

⁵²⁹ Singh R B, Niaz M A, Rastogi S S, Bajaj S, Gaoli Z, Shoumin Z. Current zinc intake and risk of diabetes and coronary artery disease and factors associated with insulin resistance in rural and urban populations of North India. J Am Coll Nutr. 1998 December;17(6):564-70.

⁵³⁰ Flaherty S F, Golenbock D T, Milham F H, Ingalls R R. CD11/CD18 leukocyte integrins: new signaling receptors for bacterial endotoxin. J Surg Res. 1997 November;73(1):85-9.

⁵³¹ Ingalls R R, Golenbock D T. CD11c/CD18, a transmembrane signaling receptor for lipopolysaccharide. J Exp Med. Apr. 1, 1995; 181(4):1473-9.

⁵³² Ingalls R R, Arnaout M A, Golenbock D T. Outside-in signaling by lipopolysaccharide through a tailless integrin. J. Immunol. Jul. 1, 1997;159(1):433-8.

⁵³³ Lammie G A, Sandercock P A, Dennis M S. Recently occluded intracranial and extracranial carotid arteries. Relevance of the unstable atherosclerotic plaque. Stroke. 1999 July;30(7):1319-25.

⁵³⁴ Chambless L E, Folsom A R, Clegg L X, Sharrett A R, Shahar E, Nieto F J, Rosamond W D, Evans G. Carotid wall thickness is predictive of incident clinical stroke: the Atherosclerosis Risk in Communities (ARIC) study. Am J Epidemiol Mar. 1, 2000;151(5):478-87.

⁵³⁵ O'Leary D H, Polak J F, Kronmal R A, Manolio T A, Burke G L, Wolfson S K. Carotid-artery intima and media thickness as a risk factor for myocardial infarction and stroke in older adults. Cardiovascular Health Study Collaborative Research Group. N EngI J. Med. Jan. 7, 1999;340(1):14-22.

⁵³⁶ Hart D N. Dendritic cells: unique leukocyte populations which control the primary immune response. Blood. Nov. 1, 1997;90(9):3245-87.

⁵³⁷ Zhuravskaya T, Maciejewski J P, Netski D M, Bruening E, Mackintosh F R, St Jeor S. Spread of human cytomegalovirus (HCMV) after infection of human hematopoietic progenitor cells: model of HCMV latency. Blood Sep. 15, 1997;90(6):2482-91.

⁵³⁸ Maciejewski J P, St Jeor S C. Human cytomegalovirus infection of human hematopoietic progenitor cells. Leuk Lymphoma 1999 March;33(1-2):1-13.

⁵³⁹ Sindre H, Tjoonnfjord G E, Rollag H, Ranneberg-Nilsen T, Veiby O P, Beck S, Degre M, Hestdal K. Human cytomegalovirus suppression of and latency in early hematopoietic progenitor cells. Blood Dec. 15, 1996;88(12):4526-33.

⁵⁴⁰ Kappelmayer J, Berecki D, Misz M, Olah L, Fekete 1, Csiba L, Blasko G. Monocytes express tissue factor in young patients with cerebral ischemia. Cerebrovasc Dis. 1998 July-August;8(4):235-9.

⁵⁴¹ Constant S L, Bottomly K. Induction of Th1 and Th2 CD4+ T cell responses: the alternative approaches. Annu Rev Immunol. 1997; 15:297-322.

⁵⁴² Rogers P R, Croft M. Peptide dose, affinity, and time of differentiation can contribute to the Th1/Th2 cytokine balance. J. Immunol. Aug. 1, 1999;163(3):1205-13.

⁵⁴³ Rovere P, Fazzini F, Sabbadini M G, Manfredi A A. Apoptosis and systemic autoimmunity: the dendritic cell connection. Eur J Histochem 2000;44(3):229-36.

⁵⁴⁴ Rovere P, Vallinoto C, Bondanza A, Crosti M C, Rescigno M, Ricciardi-Castagnoli P, Rugarli C, Manfredi A A. Bystander apoptosis triggers dendritic cell maturation and antigen-presenting function. J. Immunol. Nov. 1, 1998; 161(9):4467-71.

⁵⁴⁵ Heath W R, Kurts C, Miller J F, Carbone F R. Cross-tolerance: a pathway for inducing tolerance to peripheral tissue antigens. J Exp Med. May 18, 1998;187(10):1549-53.

⁵⁴⁶ Sallusto F, Lanzavecchia A. Mobilizing dendritic cells for tolerance, priming, and chronic inflammation. J Exp Med. 1999 Feb. 15, 1999;189(4):611-4.

⁵⁴⁷ O'Brien B A, Harmon B V, Cameron D P, Allan D J. Beta-cell apoptosis is responsible for the development of IDDM in the multiple low-dose streptozotocin model. J Pathol. 1996 February; 178(2): 176-81.

⁵⁴⁸ Lenzen S, Drinkgern J, Tiedge M. Low antioxidant enzyme gene expression in pancreatic islets compared with various other mouse tissues. Free Radic Biol Med. 1996;20(3):463-6.

⁵⁴⁹ Tiedge M, Lortz S, Drinkgern J, Lenzen S. Relation between antioxidant enzyme gene expression and antioxidative defense status of insulin-producing cells. Diabetes 1997 November;46(11):1733-42.

⁵⁵⁰ Harman A W, McKenna M, Adamson G M. Postnatal development of enzyme activities associated with protection against oxidative stress in the mouse. Biol Neonate. 1990;57(3-4):187-93.

⁵⁵¹ O'Brien B A, Harmon B V, Cameron D P, Allan D J. Nicotinamide prevents the development of diabetes in the cyclophosphamide-induced NOD mouse model by reducing beta-cell apoptosis. J Pathol. 2000 May;191(1):86-92.

⁵⁵² Hotta M, Tashiro F, Ikegami H, Niwa H, Ogihara T, Yodoi J, Miyazaki J. Pancreatic beta cell-specific expression of thioredoxin, an antioxidative and antiapoptotic protein, prevents autoimmune and streptozotocin-induced diabetes. J Exp Med. Oct. 19, 1998; 188(8): 1445-51.

⁵⁵³ Kubisch H M, Wang J, Bray T M, Phillips J P. Targeted overexpression of Cu/Zn superoxide dismutase protects pancreatic beta-cells against oxidative stress. Diabetes 1997 October;46(10):1563-6.

⁵⁵⁴ Kim J Y, Chi J K, Kim E J, Park S Y, Kim Y W, Lee S K. Inhibition of diabetes in non-obese diabetic mice by nicotinamide treatment for 5 weeks at the early age. J Korean Med Sci. 1997 August;12(4):293-7.

⁵⁵⁵ Reddy S, Bibby N J, Elliott R B. Early nicotinamide treatment in the NOD mouse: effects on diabetes and insulitis suppression and autoantibody levels. Diabetes Res. 1990 October;15(2):95-102.

⁵⁵⁶ Faust A, Burkart V, Ulrich H, Weischer C H, Kolb H. Effect of lipoic acid on cyclophosphamide-induced diabetes and insulitis in non-obese diabetic mice. Int J Immunopharmacol. 1994 January;16(1):61-6.

⁵⁵⁷ Rabinovitch A, Suarez W L, Power R F. Lazaroid antioxidant reduces incidence of diabetes and insulitis in nonobese diabetic mice. J Lab Clin Med. 1993 April;121(4):603-7.

⁵⁵⁸ Mori Y, Suko M, Okudaira H, Matsuba 1, Tsuruoka A, Sasaki A, Yokoyama H, Tanase T, Shida T, Nishimura M, et al. Preventive effects of cyclosporin on diabetes in NOD mice. Diabetologia. 1986 April;29(4):244-7.

⁵⁵⁹ Laupacis A, Stiller C R, Gardell C, Keown P, Dupre J, Wallace A C, Thibert P. Cyclosporin prevents diabetes in B B Wistar rats. Lancet. Jan. 1, 1983;1(8314-5):10-2.

⁵⁶⁰ Sevilla N, Kunz S, Holz A, Lewicki H, Homann D, Yamada H, Campbell K P, de La Torre J C, Oldstone M B. Immunosuppression and resultant viral persistence by specific viral targeting of dendritic cells. J Exp Med. Nov. 6, 2000;192(9):1249-60.

⁵⁶¹ Roman L M, Simons L F, Hammer R E, Sambrook J F, Gething M J. The expression of influenza virus hemagglutinin in the pancreatic beta cells oftransgenic mice results in autoimmune diabetes. Cell. May 4, 1990;61(3):383-96.

⁵⁶² Sarvetnick N, Shizuru J, Liggitt D, Martin L, McIntyre B, Gregory A, Parslow T, Stewart T. Loss of pancreatic islet tolerance induced by beta-cell expression of interferon-gamma. Nature. Aug. 30, 1990;346(6287):844-7.

⁵⁶³ Yamaoka T, Yano M, Idehara C, Yamada T, Tomonari S, Moritani M, li S, Yoshimoto K, Hata J, Itakura M. Apoptosis and remodelling of beta cells by paracrine interferon-gamma without insulitis in transgenic mice. Diabetologia. 1999 May;42(5):566-73.

⁵⁶⁴ Ohashi P S, Oehen S, Buerki K, Pircher H, Ohashi C T, Odermatt B, Malissen B, Zinkernagel R M, Hengartner H. Ablation of “tolerance” and induction of diabetes by virus infection in viral antigen transgenic mice. Cell. Apr. 19, 1991;65(2):305-17.

⁵⁶⁵ Oldstone M B, Nerenberg M, Southern P, Price J, Lewicki H. Virus infection triggers insulin-dependent diabetes mellitus in a transgenic model: role of anti-self (virus) immune response. Cell. Apr. 19, 1991;65(2):319-31.

⁵⁶⁶ von Herrath M, Holz A. Pathological changes in the islet milieu precede infiltration of islets and destruction of beta-cells by autoreactive lymphocytes in a transgenic model of virus-induced IDDM. J Autoimmun. 1997 June; 10(3):231-8.

⁵⁶⁷ Garza K M, Chan S M, Suri R, Nguyen L T, Odermatt B, Schoenberger S P, Ohashi P S. Role of antigen-presenting cells in mediating tolerance and autoimmunity. J Exp Med. Jun. 5, 2000;191(11):2021-7.

⁵⁶⁸ Pradier O, Willems F, Abramowicz D, Schandene L, de Boer M, Thielemans K, Capel P, Goldman M. CD40 engagement induces monocyte procoagulant activity through an interleukin-10 resistant pathway. Eur J. Immunol. 1996 December;26(12):3048-54.

⁵⁶⁹ Mach F, Schonbeck U, Bonnefoy J Y, Pober J S, Libby P. Activation of monocyte/macrophage functions related to acute atheroma complication by ligation of CD40: induction of collagenase, stromelysin, and tissue factor. Circulation. Jul. 15, 1997;96(2):396-9.

⁵⁷⁰ Schonbeck U, Mach F, Sukhova G K, Herman M, Graber P, Kehry M R, Libby P. CD40 ligation induces tissue factor expression in human vascular smooth muscle cells. Am J Pathol. 2000 January; 156(1):7-14.

⁵⁷¹ Moodycliffe A M, Shreedhar V, Ullrich S E, Walterscheid J, Bucana C, Kripke M L, Flores-Romo L. CD40-CD40 ligand interactions in vivo regulate migration of antigen-bearing dendritic cells from the skin to draining lymph nodes. J Exp Med. Jun. 5, 2000;191(11):2011-20.

⁵⁷² Ehl S, Hombach J, Aichele P, Rulicke T, Odermatt B, Hengartner H, Zinkernagel R, Pircher H. Viral and bacterial infections interfere with peripheral tolerance induction and activate CD8+ T cells to cause immunopathology. J Exp Med. Mar. 2, 1998; 187(5):763-74.

⁵⁷³ Zhuravskaya T, Maciejewski J P, Netski D M, Bruening E, Mackintosh F R, St Jeor S. Spread of human cytomegalovirus (HCMV) after infection of human hematopoietic progenitor cells: model of HCMV latency. Blood Sep. 15, 1997;90(6):2482-91.

⁵⁷⁴ Ilowite N T. Premature atherosclerosis in systemic lupus erythematosus. J Rheumatol. 2000 April;27 Suppl 58:15-9.

⁵⁷⁵ Urowitz M, Gladman D, Bruce I. Atherosclerosis and Systemic Lupus Erythematosus. Curr Rheumatol Rep 2000 February;2(1):19-23

⁵⁷⁶ Dobado-Berrios P M, Lopez-Pedrera C, Velasco F, Aguirre M A, Torres A, Cuadrado M J. Increased levels of tissue factor mRNA in mononuclear blood cells of patients with primary antiphospholipid syndrome. Thromb Haemost 1999 December;82(6):1578-82.

⁵⁷⁷ Ludewig B, Odermatt B, Landmann S, Hengartner H, Zinkernagel R M. Dendritic cells induce autoimmune diabetes and maintain disease via de novo formation of local lymphoid tissue. J Exp Med. Oct. 19, 1998;188(8):1493-501.

⁵⁷⁸ Fearnley D B, Whyte L F, Carnoutsos S A, Cook A H, Hart D N. Monitoring human blood dendritic cell numbers in normal individuals and in stem cell transplantation. Blood. Jan. 15, 1999;93(2):728-36.

⁵⁷⁹ Barratt-Boyes S M, Zimmer M I, Harshyne L A, Meyer E M, Watkins S C, Capuano S, Murphey-Corb M, Falo L D, Donnenberg A D. Maturation and trafficking of monocyte-derived dendritic cells in monkeys: implications for dendritic cell-based vaccines. J. Immunol. Mar. 1, 2000;164(5):2487-95.

⁵⁸⁰ Hermans I F, Ritchie D S, Yang J, Roberts J M, Ronchese F. CD8+ T cell-dependent elimination of dendritic cells in vivo limits the induction of antitumor immunity. J. Immunol. Mar. 15, 2000; 164(6):3095-101.

⁵⁸¹ Ludewig B, Ochsenbein A F, Odermatt B, Paulin D, Hengartner H, Zinkernagel R M. Immunotherapy with dendritic cells directed against tumor antigens shared with normal host cells results in severe autoimmune disease. J Exp Med. Mar. 6, 2000;191(5):795-804.

⁵⁸² Roskrow M A, Dilloo D, Suzuki N, Zhong W, Rooney C M, Brenner M K. Autoimmune disease induced by dendritic cell immunization against leukemia. Leuk Res 1999 June;23(6):549-57.

⁵⁸³ Lipton H L, Pritchard A E, Calenoff M A. Attenuation of neurovirulence of Theiler's murine encephalomyelitis virus strain GDVII is not sufficient to establish persistence in the central nervous system. J Gen Virol. 1998 May;79 (Pt 5): 1001-4.

⁵⁸⁴ Tsunoda I, Kurtz C I, Fujinami R S. Apoptosis in acute and chronic central nervous system disease induced by Theiler's murine encephalomyelitis virus. Virology. Feb. 17, 1997;228(2):388-93.

⁵⁸⁵ Ha-Lee Y M, Dillon K, Kosaras B, Sidman R, Revell P, Fujinami R, Chow M. Mode of spread to and within the central nervous system after oral infection of neonatal mice with the DA strain of Theiler's murine encephalomyelitis virus. J. Virol. 1995 November;69(11):7354-61.

⁵⁸⁶ Lipton H L, Twaddle G, Jelachich M L. The predominant virus antigen burden is present in macrophages in Theiler's murine encephalomyelitis virus-induced demyelinating disease. J. Virol. 1995 April;69(4):2525-33.

⁵⁸⁷ Miller S D, Vanderlugt C L, Begolka W S, Pao W, Yauch R L, Neville K L, Katz-Levy Y, Carrizosa A, Kim B S. Persistent infection with Theiler's virus leads to CNS autoimmunity via epitope spreading. Nat Med. 1997 October;3(10):1133-6.

⁵⁸⁸ Signore A, Chianelli M, Parisella M G, Capriotti G, Giacalone P, Di Leve G, Barone R. In vivo imaging of insulitis in autoimmune diabetes. J Endocrinol Invest. 1999 February;22(2):151-8.

⁵⁸⁹ Foulis A K, McGill M, Farquharson M A. Insulitis in type I (insulin-dependent) diabetes mellitus in man—macrophages, lymphocytes, and interferon-gamma containing cells. J Pathol. 1991 October; 165(2):97-103.

⁵⁹⁰ Foulis A K, Stewart J A. The pancreas in recent-onset type I (insulin-dependent) diabetes mellitus: insulin content of islets, insulitis and associated changes in the exocrine acinar tissue. Diabetologia. 1984 June;26(6):456-61.

⁵⁹¹ Roivainen M, Rasilainen S, Ylipaasto P, Nissinen R, Ustinov J, Bouwens L, Eizirik D L, Hovi T, Otonkoski T. Mechanisms of coxsackievirus-induced damage to human pancreatic beta-cells. J Clin Endocrinol Metab 2000 January;85(1):432-40.

⁵⁹² Andreoletti L, Hober D, Hober-Vandenberghe C, Fajardy 1, Belaich S, Lambert V, Vantyghem M C, Lefebvre J, Wattre P. Coxsackie B virus infection and beta cell autoantibodies in newly diagnosed IDDM adult patients. Clin Diagn Virol. 1998 April;9(2-3):125-33.

⁵⁹³ Andreoletti L, Hober D, Hober-Vandenberghe C, Belaich S, Vantyghem M C, Lefebvre J, Wattre P. Detection of coxsackie B virus RNA sequences in whole blood samples from adult patients at the onset of type I diabetes mellitus. J Med Virol. 1997 June;52(2):121-7.

⁵⁹⁴ Frisk G, Diderholm H. Antibody responses to different strains of coxsackie B4 virus in patients with newly diagnosed type I diabetes mellitus or aseptic meningitis. J. Infect. 1997 May;34(3):205-10.

⁵⁹⁵ Clements G B, Galbraith D N, Taylor K W. Coxsackie B virus infection and onset of childhood diabetes. Lancet. Jul. 22, 1995;346(8969):221-3.

⁵⁹⁶ De Simone R, Giampaolo A, Giometto B, Gallo P, Levi G, Peschle C, Aloisi F. The costimulatory molecule B7 is expressed on human microglia in culture and in multiple sclerosis acute lesions. J Neuropathol Exp Neurol. 1995 March;54(2):175-87.

⁵⁹⁷ Windhagen A, Newcombe J, Dangond F, Strand C, Woodroofe M N, Cuzner M L, Hafler D A. Expression of costimulatory molecules B7-1 (CD80), B7-2 (CD86), and interleukin 12 cytokine in multiple sclerosis lesions. J Exp Med. Dec. 1, 1995;182(6):1985-96.

⁵⁹⁸ Boven L A, Montagne L, Nottet H S, De Groot C J. Macrophage inflammatory protein-1 alpha (MIP-lalpha), MIP-1 beta, and RANTES mRNA semiquantification and protein expression in active demyelinating multiple sclerosis (MS) lesions. Clin Exp Immunol. 2000 November;122(2):257-63.

⁵⁹⁹ Khanna K V, Markham R B. A perspective on cellular immunity in the elderly. Clin Infect Dis 1999 April;28(4):710-3.

⁶⁰⁰ Ginaldi L, De Martinis M, D'Ostilio A, Marini L, Loreto M F, Martorelli V, Quaglino D. The immune system in the elderly: II. Specific cellular immunity. Immunol Res 1999;20(2):109-15.

⁶⁰¹ Liedtke W, Opalka B, Zimmermann C W, Lignitz E. Age distribution of latent herpes simplex virus 1 and varicella-zoster virus genome in human nervous tissue. J Neurol Sci. 1993 May; 116(1):6-11.

⁶⁰² Beer W E, Smith A E, Kassab J Y, Smith P H, Rowland Payne C M. Concomitance of psoriasis and atopic dermatitis. Dermatology. 1992;184(4):265-70.

⁶⁰³ Abrams J R, Kelley S L, Hayes E, Kikuchi T, Brown M J, Kang S, Lebwohl M G, Guzzo C A, Jegasothy B V, Linsley P S, Krueger J G. Blockade of T lymphocyte costimulation with cytotoxic T lymphocyte-associated antigen 4-immunoglobulin (CTLA4Ig) reverses the cellular pathology of psoriatic plaques, including the activation of keratinocytes, dendritic cells, and endothelial cells. J Exp Med. Sep. 4, 2000;192(5):681-94.

⁶⁰⁴ Ohki O, Yokozeki H, Katayama 1, Umeda T, Azuma M, Okumura K, Nishioka K. Functional CD86 (B7-2/B70) is predominantly expressed on Langerhans cells in atopic dermatitis. Br J Dermatol 1997 June;136(6):838-45.

⁶⁰⁵ Asadullah K, Prosch S, Audring H, Buttnerova 1, Volk H D, Sterry W, Docke W D. A high prevalence of cytomegalovirus antigenaemia in patients with moderate to severe chronic plaque psoriasis: an association with systemic tumour necrosis factor alpha overexpression. Br J. Dermatol. 1999 July;141(1):94-102.

⁶⁰⁶ Steigleder G K, Rasokat H, Wemmer U. [Psoriasis in HTLV-III-induced immunologic defect—status of cellular immunity and immunohistologic findings]. Z Hautkr Dec. 1, 1986;61(23):1671-8. [Article in German]

⁶⁰⁷ Montazeri A, Kanitakis J, Bazex J. Psoriasis and HIV infection. Int J. Dermatol. 1996 July;35(7):475-9.

⁶⁰⁸ Fischer T, Schworer H, Vente C, Reich K, Ramadori G. Clinical improvement of HIV-associated psoriasis parallels a reduction of HIV viral load induced by effective antiretroviral therapy. AIDS. Apr. 1, 1999;13(5):628-9.

⁶⁰⁹ Vermaelen K Y, Carro-Muino 1, Lambrecht B N, Pauwels R A. Specific Migratory Dendritic Cells Rapidly Transport Antigen from the Airways to the Thoracic Lymph Nodes. J Exp Med Jan. 1, 2001;193(1):51-60.

⁶¹⁰ Lambrecht B N, Pauwels R A, Fazekas De St Groth B. Induction of rapid T cell activation, division, and recirculation by intratracheal injection of dendritic cells in a TCR transgenic model. J. Immunol. Mar. 15, 2000; 164(6):2937-46.

⁶¹¹ Lambrecht B N, De Veerman M, Coyle A J, Gutierrez-Ramos J C, Thielemans K, Pauwels R A. Myeloid dendritic cells induce Th2 responses to inhaled antigen, leading to eosinophilic airway inflammation. J Clin Invest. 2000 August;106(4):551-9.

⁶¹² Lambrecht B N, Salomon B, Klatzmann D, Pauwels R A. Dendritic cells are required for the development of chronic eosinophilic airway inflammation in response to inhaled antigen in sensitized mice. J. Immunol. Apr. 15, 1998;160(8):4090-7.

⁶¹³ Mathur M, Herrmann K, Qin Y, Gulmen F, Li X, Krimins R, Weinstock J, Elliott D, Bluestone J A, Padrid P. CD28 interactions with either CD80 or CD86 are sufficient to induce allergic airway inflammation in mice. Am J Respir Cell Mol. Biol. 1999 October;21(4):498-509.

⁶¹⁴ Haczku A, Takeda K, Redai I, Hamelmann E, Cieslewicz G, Joetham A, Loader J, Lee J J, Irvin C, Gelfand E W. Anti-CD86 (B7.2) treatment abolishes allergic airway hyperresponsiveness in mice. Am J Respir Crit Care Med. 1999 May;159(5 Pt 1):1638-43.

⁶¹⁵ Padrid P A, Mathur M, Li X, Herrmann K, Qin Y, Cattamanchi A, Weinstock J, Elliott D, Sperling A I, Bluestone J A. CTLA4Ig inhibits airway eosinophilia and hyperresponsiveness by regulating the development of Th1/Th2 subsets in a murine model of asthma. Am J Respir Cell Mol. Biol. 1998 April; 18(4):453-62.

⁶¹⁶ Keane-Myers A M, Gause W C, Finkelman F D, Xhou X D, Wills-Karp M. Development of murine allergic asthma is dependent upon B7-2 costimulation. J. Immunol. Jan. 15, 1998;160(2):1036-43.

⁶¹⁷ Balsa A, Dixey J, Sansom D M, Maddison P J, Hall N D. Differential expression of the costimulatory molecules B7.1 (CD80) and B7.2 (CD86) in rheumatoid synovial tissue. Br J Rheumatol 1996 January;35(1):33-7.

⁶¹⁸ Liu M F, Kohsaka H, Sakurai H, Azuma M, Okumura K, Saito I, Miyasaka N. The presence of costimulatory molecules CD86 and CD28 in rheumatoid arthritis synovium. Arthritis Rheum 1996 January;39(1):110-4.

⁶¹⁹ Watanabe H, Inaba M, Adachi Y, Sugiura K, Hisha H, Iguchi T, Ito T, Yasumizu R, Inaba K, Yamashita T, Ikehara S. Experimental autoimmune thyroiditis induced by thyroglobulin-pulsed dendritic cells. Autoimmunity 1999;31 (4):273-82.

⁶²⁰ Tandon N, Metcalfe R A, Barnett D, Weetman A P. Expression of the costimulatory molecule B7/BB1 in autoimmune thyroid disease. Q J. Med. 1994 April;87(4):231-6.

⁶²¹ Muthalif M M, Benter I F, Karzoun N, Fatima S, Harper J, Uddin M R, Malik K U. 20-Hydroxyeicosatetraenoic acid mediates calcium/calmodulin-dependent protein kinase 11-induced mitogen-activated protein kinase activation in vascular smooth muscle cells. Proc Natl Acad Sci U S A. Oct. 13, 1998;95(21):12701-6.

⁶²² Wen Y, Nadler J L, Gonzales N, Scott S, Clauser E, Natarajan R. Mechanisms of ANG II-induced mitogenic responses: role of 12-lipoxygenase and biphasic MAP kinase. Am J. Physiol. 1996 October;271(4 Pt 1):C1212-20.

⁶²³ Rao G N, Baas A S, Glasgow W C, Eling T E, Runge M S, Alexander R W. Activation of mitogen-activated protein kinases by arachidonic acid and its metabolites in vascular smooth muscle cells. J. Biol. Chem. Dec. 23, 1994;269(51):32586-91.

⁶²⁴ Chen J K, Wang D W, Falck J R, Capdevila J, Harris R C. Transfection of an active cytochrome P450 arachidonic acid epoxygenase indicates that 14,15-epoxyeicosatrienoic acid functions as an intracellular second messenger in response to epidermal growth factor. J. Biol. Chem. Feb. 19, 1999;274(8):4764-9.

⁶²⁵ Bylund J, Ericsson J, Oliw E H. Analysis of cytochrome P450 metabolites of arachidonic and linoleic acids by liquid chromatography-mass spectrometry with ion trap M S. Anal Biochem. Dec. 1, 1998;265(1):55-68.

⁶²⁶ Imaoka S, Wedlund P J, Ogawa H, Kimura S, Gonzalez F J, Kim H Y. Identification of CYP2C23 expressed in rat kidney as an arachidonic acid epoxygenase. J Pharmacol Exp Ther. 1993 November;267(2): 1012-6.

⁶²⁷ Zeldin D C, Plitman J D, Kobayashi J, Miller R F, Snapper J R, Falck J R, Szarek J L, Philpot R M, Capdevila J H. The rabbit pulmonary cytochrome P450 arachidonic acid metabolic pathway: characterization and significance. J Clin Invest. 1995 May;95(5):2150-60.

⁶²⁸ Rifkind A B, Lee C, Chang T K, Waxman D J. Arachidonic acid metabolism by human cytochrome P450s 2C8, 2C9, 2E1, and IA2: regioselective oxygenation and evidence for a role for CYP2C enzymes in arachidonic acid epoxygenation in human liver microsomes. Arch Biochem Biophys. Jul. 10, 1995;320(2):380-9.

⁶²⁹ Luo G, Zeldin D C, Blaisdell J A, Hodgson E, Goldstein J A. Cloning and expression of murine CYP2Cs and their ability to metabolize arachidonic acid. Arch Biochem Biophys. Sep. 1, 1998;357(1):45-57.

⁶³⁰ Keeney D S, Skinner C, Travers J B, Capdevila J H, Nanney L B, King L E Jr, Waterman M R. Differentiating keratinocytes express a novel cytochrome P450 enzyme, CYP2B 19, having arachidonate monooxygenase activity. J. Biol. Chem. Nov. 27, 1998;273(48):32071-9.

⁶³¹ Kidd R S, Straughn A B, Meyer M C, Blaisdell J, Goldstein J A, Dalton J T. Pharmacokinetics of chlorpheniramine, phenytoin, glipizide and nifedipine in an individual homozygous for the CYP2C9*3 allele. Pharmacogenetics. 1999 February;9(1):71-80.

⁶³² Ring B J, Binkley S N, Vandenbranden M, Wrighton S A. In vitro interaction of the antipsychotic agent olanzapine with human cytochromes P450 CYP2C9, CYP2C19, CYP2D6 and CYP3A. Br J Clin Pharmacol. 1996 March;41(3):181-6.

⁶³³ Miners J O, Birkett D J. Cytochrome P4502C9: an enzyme of major importance in human drug metabolism. Br J Clin Pharmacol. 1998 June;45(6):525-38. Review.

⁶³⁴ Egger J, Brett E M. Effects of sodium valproate in 100 children with special reference to weight. Br Med J (Clin Res Ed). Aug. 29, 1981;283(6291):577-81.

⁶³⁵ Campbell I W, Menzies D G, Chalmers J, McBain A M, Brown I R. One year comparative trial of metformin and glipizide in type 2 diabetes mellitus. Diabete Metab 1994 July-August;20(4):394-400.

⁶³⁶ Petersen K U. Review article: omeprazole and the cytochrome P450 system. Aliment Pharmacol Ther 1995 February;9(1):1-9.

⁶³⁷ Meyer U A. Interaction of proton pump inhibitors with cytochromes P450: consequences for drug interactions. Yale J Biol Med 1996 May-June;69(3):203-9.

⁶³⁸ Hogan R E, Bertrand M E, Deaton R L, Sommerville K W. Total percentage body weight changes during add-on therapy with tiagabine, carbamazepine and phenytoin. Epilepsy Res. 2000 August;41(1):23-8.

⁶³⁹ Mattson R H, Cramer J A, Collins J F. A comparison of valproate with carbamazepine for the treatment of complex partial seizures and secondarily generalized tonic-clonic seizures in adults. The Department of Veterans Affairs Epilepsy Cooperative Study No. 264 Group. N Engl J. Med. Sep. 10, 1992;327(11):765-71.

⁶⁴⁰ Sadeque AJM, Fisher M B, Korzekwa K R, Gonzalez F J, Rettie A E. Human CYP2C9 and CYP2A6 mediate formation of the hepatotoxin 4-ene-valproic acid. J Pharmacol Exp Ther. 1997 November;283(2):698-703.

⁶⁴¹ Bruni J, Wilder B J. Valproic acid. Review of a new antiepileptic drug. Arch Neurol 1979 July;36(7):393 8.

⁶⁴² Zaccara G, Campostrini R, Paganini M, Messori A, Valenza T, Arnetoli G, Zappoli R. Long-term treatment with sodium valproate: monitoring of venous ammonia concentrations and adverse effects. Ther Drug Monit. 1987;9(1):34-40.

⁶⁴³ Sharpe C, Buchanan N. Juvenile myoclonic epilepsy: diagnosis, management and outcome. Med J Aust. Feb. 6, 1995;162(3):1334.

⁶⁴⁴ Song J C, White C M. Pharmacologic, pharmacokinetic, and therapeutic differences among angiotensin II receptor antagonists. Pharmacotherapy. 2000 February;20(2):130-9. Review

⁶⁴⁵ Meadowcroft A M, Williamson K M, Patterson J H, Hinderliter A L, Pieper J A. The effects of fluvastatin, a CYP2C9 inhibitor, on losartan pharmacokinetics in healthy volunteers. J Clin Pharmacol. 1999 April;39(4):418-24.

⁶⁴⁶ Camargo M J, von Lutterotti N, Pecker M S, James G D, Timmermans P B, Laragh J H. DuP 753 increases survival in spontaneously hypertensive stroke-prone rats fed a high sodium diet. Am J Hypertens. 1991 April;4(4 Pt 2):341 S-345S.

⁶⁴⁷ Transon C, Leemann T, Dayer P. In vitro comparative inhibition profiles of major human drug metabolising cytochrome P450 isozymes (CYP2C9, CYP2D6 and CYP3A4) by HMG-CoA reductase inhibitors. Eur J Clin Pharmacol. 1996;50(3):209-15.

⁶⁴⁸ Matthews P G, Wahlqvist M L, Marks S J, Myers K A, Hodgson J M. Improvement in arterial stiffness during hypolipidaemic therapy is offset by weight gain. Int J Obes Relat Metab Disord. 1993 October; 17(10):579-83.

⁶⁴⁹ Osser D N, Najarian D M, Dufresne R L. Olanzapine increases weight and serum triglyceride levels. J Clin Psychiatry. 1999 November;60(11):767-70.

⁶⁵⁰ Koran L M, Ringold A L, Elliott M A. Olanzapine augmentation for treatment-resistant obsessive-compulsive disorder. J Clin Psychiatry. 2000 July;61(7):514-7.

⁶⁵¹ Fang J, Coutts R T, McKenna K F, Baker G B. Elucidation of individual cytochrome P450 enzymes involved in the metabolism of clozapine. Naunyn Schmiedebergs Arch Pharmacol. 1998 November;358(5):592-9.

⁶⁵² Prior T I, Chue P S, Tibbo P, Baker G B. Drug metabolism and a typical antipsychotics. Eur Neuropsychopharmacol. 1999 June;9(4):301-9. Review.

⁶⁵³ Olesen O V, Linnet K. Fluvoxamine-Clozapine drug interaction: inhibition in vitro of five cytochrome P450 isoforms involved in clozapine metabolism. J Clin Psychopharmacol. 2000 February;20(1):35-42.

⁶⁵⁴ Schmider J, Greenblatt D J, von Moltke L L, Karsov D, Shader R1. Inhibition of CYP2C9 by selective serotonin reuptake inhibitors in vitro: studies of phenytoin p-hydroxylation. Br J Clin Pharmacol. 1997 November;44(5):495-8.

⁶⁵⁵ Harvey B H, Bouwer C D. Neuropharmacology of paradoxic weight gain with selective serotonin reuptake inhibitors. Clin Neuropharmacol. 2000 March-April;23(2):90-7. Review.

⁶⁵⁶ Sansone R A, Wiederman M W, Shrader J A. Naturalistic study of the weight effects of amitriptyline, fluoxetine, and sertraline in an outpatient medical setting. J Clin Psychopharmacol. 2000 April;20(2):272-4.

⁶⁵⁷ Michelson D, Amsterdam J D, Quitkin F M, Reimherr F W, Rosenbaum J F, Zajecka J, Sundell K L, Kim Y, Beasley C M Jr. Changes in weight during a 1-year trial of fluoxetine. Am J Psychiatry. 1999 August; 156(8): 1170-6.

⁶⁵⁸ Darga L L, Carroll-Michals L, Botsford S J, Lucas C P. Fluoxetine's effect on weight loss in obese subjects. Am J Clin Nutr. 1991 August;54(2):321-5.

⁶⁵⁹ Lasker J M, Wester M R, Aramsombatdee E, Raucy J L. Characterization of CYP2C 19 and CYP2C9 from human liver: respective roles in microsomal tolbutamide, S-mephenytoin, and omeprazole hydroxylations. Arch Biochem Biophys. May 1, 1998;353(1):16-28.

⁶⁶⁰ Wissler R W, Borensztajn J, Rubenstein A, Getz G, Vesselinovitch D The effects of tolbutamide on the development of atherosclerosis in rhesus monkeys fed an average American table-prepared diet. Adv Exp Med Biol. 1975;63:379-80.

⁶⁶¹ Ballagi-Pordany G, Koltai M Z, Aranyi Z, Pogatsa G. Direct effect of hypoglycemic sulphonylureas on the cardiovascular system of dogs. Diabetes Res Clin Pract. 1991 January;11(1):47-52.

⁶⁶² Grimm S W, Dyroff M C. Inhibition of human drug metabolizing cytochromes P450 by anastrozole, a potent and selective inhibitor of aromatase. Drug Metab Dispos. 1997 May;25(5):598-602.

⁶⁶³ Wiseman L R, Adkins J C. Anastrozole. A review of its use in the management of postmenopausal women with advanced breast cancer. Drugs Aging. 1998 October;13(4):321-32. Review.

⁶⁶⁴ Lonning P E. Aromatase inhibitors and their future role in post-menopausal women with early breast cancer. Br J Cancer. 1998 September;78 Suppl 4:12-5.

⁶⁶⁵ Buzdar A U. Anastrozole: a new addition to the armamentarium against advanced breast cancer. Am J Clin Oncol. 1998 April;21(2):161-6. Review.

⁶⁶⁶ Jonat W. Clinical overview of anastrozole—a new selective oral aromatase inhibitor. Oncology. 1997;54 Suppl 2:15-8. Review.

⁶⁶⁷ Buzdar A U, Jonat W, Howell A, Plourde P V. ARIMIDEX: a potent and selective aromatase inhibitor for the treatment of advanced breast cancer. J Steroid Biochem Mol. Biol. 1997 April;61(3-6):145-9. Review.

⁶⁶⁸ Hannaford M. Role of new selective aromatase inhibitor in therapy for metastatic breast cancer in postmenopausal women. Nurse Pract. 1997 March;22(3):195-6, 201-2.

⁶⁶⁹ Buzdar A U, Jones S E, Vogel C L, Wolter J, Plourde P, Webster A. A phase III trial comparing anastrozole (1 and 10 milligrams), a potent and selective aromatase inhibitor, with megestrol acetate in postmenopausal women with advanced breast carcinoma. Arimidex Study Group. Cancer. Feb. 15, 1997;79(4):730-9.

⁶⁷⁰ Buzdar A, Jonat W, Howell A, Jones S E, Blomqvist C, Vogel C L, Eiermann W, Wolter J M, Azab M, Webster A, Plourde P V. Anastrozole, a potent and selective aromatase inhibitor, versus megestrol acetate in postmenopausal women with advanced breast cancer: results of overview analysis of two phase III trials. Arimidex Study Group. J Clin Oncol. 1996 July;14(7):2000-11.

⁶⁷¹ Jonat W, Howell A, Blomqvist C, Eiermann W, Winblad G, Tyrrell C, Mauriac L, Roche H, Lundgren S, Hellmund R, Azab M. A randomised trial comparing two doses of the new selective aromatase inhibitor anastrozole (Arimidex) with megestrol acetate in postmenopausal patients with advanced breast cancer. Eur J Cancer. 1996 March;32A(3):404-12.

⁶⁷² Khaliq Y, Gallicano K, Seguin I, Fyke K, Carignan G, Bulman D, Badley A, Cameron D W. Single and multiple dose pharmacokinetics of nelfinavir and CYP2CI9 activity in human immunodeficiency virus-infected patients with chronic liver disease. Br J Clin Pharmacol. 2000 August;50(2):108-15.

⁶⁷³ Lillibridge J H, Liang B H, Kerr B M, Webber S, Quart B, Shetty B V, Lee C A. Characterization of the selectivity and mechanism of human cytochrome P450 inhibition by the human immunodeficiency virus-protease inhibitor nelfinavir mesylate. Drug Metab Dispos. 1998 July;26(7):609-16.

⁶⁷⁴ Muirhead G J, Wulff M B, Fielding A, KIeinermans D, Buss N. Pharmacokinetic interactions between sildenafil and saquinavir/ritonavir. Br J Clin Pharmacol. 2000 August;50(2):99-107.

⁶⁷⁵ Kumar G N, Dykstra J, Roberts E M, Jayanti V K, Hickman D, Uchic J, Yao Y, Surber B, Thomas S, Granneman GRPotent inhibition of the cytochrome P-450 3A-mediated human liver microsomal metabolism of a novel HIV protease inhibitor by ritonavir: A positive drug-drug interaction. Drug Metab Dispos. 1999 August;27(8):902-8.

⁶⁷⁶ Kumar G N, Rodrigues A D, Buko A M, Denissen J F. Cytochrome P450-mediated metabolism of the HIV-1 protease inhibitor ritonavir (ABT-538) in human liver microsomes. J Pharmacol Exp Ther. 1996 April;277(1):423-31.

⁶⁷⁷ Eagling V A, Back D J, Barry M G. Differential inhibition of cytochrome P450 isoforms by the protease inhibitors, ritonavir, saquinavir and indinavir. Br J Clin Pharmacol. 1997 August;44(2): 190-4.

⁶⁷⁸ Fung H B, Kirschenbaum H L, Hameed R. Amprenavir: a new human immunodeficiency virus type I protease inhibitor. Clin Ther. 2000 May;22(5):549-72. Review.

⁶⁷⁹ Fisslthaler B, Hinsch N, Chataigneau T, Popp R, Kiss L, Busse R, Fleming I. Nifedipine increases cytochrome P4502C expression and endothelium-derived hyperpolarizing factor-mediated responses in coronary arteries. Hypertension. 2000 August;36(2):270-5.

⁶⁸⁰ Krakoff LREffectiveness of nifedipine gastrointestinal therapeutic system for treatment of hypertension: results of the MATH Trial. J Cardiovasc Pharmacol. 1993;21 Suppl 2:S14-7.

⁶⁸¹ Maccario M, Oleandri S E, Avogadri E, Rossetto R, Grottoli S, Procopio M, Camanni F, Ghigo E. Effects of 3-month nifedipine treatment on endocrine-metabolic parameters in patients with abdominal obesity and mild hypertension. J Endocrinol Invest. 1998 January;21(1):56-63.

⁶⁸² Andronico G, Piazza G, Mangano M T, Mule G, Carone M B, Cerasola G. Nifedipine vs. enalapril in treatment of hypertensive patients with glucose intolerance. J Cardiovasc Pharmacol. 1991;18 Suppl 10:S52-4.

⁶⁸³ Barreiro P, Soriano V, Blanco F, Casimiro C, de la Cruz J J, Gonzalez-Lahoz J. Risks and benefits of replacing protease inhibitors by nevirapine in HIV-infected subjects under long-term successful triple combination therapy. AIDS. May 5, 2000;14(7):807-12.

⁶⁸⁴ Mulligan K, Grunfeld C, Tai V W, Algren H, Pang M, Chernoff D N, Lo J C, Schambelan M. Hyperlipidemia and insulin resistance are induced by protease inhibitors independent of changes in body composition in patients with HIV infection. J Acquir Immune Defic Syndr. Jan. 1, 2000;23(1):35-43.

⁶⁸⁵ Gervasoni C, Ridolfo A L, Trifiro G, Santambrogio S, Norbiato G, Musicco M, Clerici M, Galli M, Moroni M. Redistribution of body fat in HIV-infected women undergoing combined antiretroviral therapy. AIDS. Mar. 11, 1999;13(4):465-71.

⁶⁸⁶ Carr A, Copper D A. A randomized, multicenter study of protease inhibitor (PI) substitution in aviremic patients with antiretroviral (ARV) lipodystrophy syndrome. 7th Conference on Retroviruses and Opportunistic Infections. 2000 (Abstract).

⁶⁸⁷ Martinez E, Blanco J L, Garcia M A, Buira E, Bianchi L, Conget I, Casamitjana R, Gatell J M. Impact of switching from HIV-1 protease Inhibitors (PI) to efavirenz (EFV) in patients with lipodystrophy. 7th Conference on Retroviruses and Opportunistic Infections. 2000 (Abstract).

⁶⁸⁸ Passalaris J D, Sepkowitz K A, Glesby M J. Coronary artery disease and human immunodeficiency virus infection. Clin Infect Dis. 2000 September;31 (3):787-97.

⁶⁸⁹ Andrews G K, Adamson E D. Butyrate selectively activates the metallothionein gene in teratocarcinoma cells and induces hypersensitivity to metal induction. Nuclein Acids Research. 1987 15(13): 5461-5475.

⁶⁹⁰ Thomas D J, Angle C R, Swanson S A, Caffrey T C. Effect of sodium butyrate on metallothionein induction and cadmium cytotoxicity in ROS 17/2.8 cells. Toxicology Feb. 11, 1991;66(1):35-46.

⁶⁹¹ Liu J, McKim J M Jr, Liu Y P, Klaassen C D. Effects of butyrate homologues on metallothionein induction in rat primary hepatocyte cultures. In vitro Cell Dev Biol 1992 May;28A(5):320-6.

⁶⁹² Ludwig D S, Pereira M A, Kroenke C H, Hilner J E, Van Horn L, Slattery M L, Jacobs D R Jr. Dietary fiber, weight gain, and cardiovascular disease risk factors in young adults. JAMA. Oct. 27, 1999;282(16): 1539-46.

⁶⁹³ Rigaud D, Ryttig K R, Angel L A, Apfelbaum M. Overweight treated with energy restriction and a dietary fibre supplement: a 6-month randomized, double-blind, placebo-controlled trial. Int J Obes. 1990 September;14(9):763-9.

⁶⁹⁴ Ryttig K R, Tellnes G, Haegh L, Boe E, Fagerthun H. A dietary fibre supplement and weight maintenance after weight reduction: a randomized, double-blind, placebo-controlled long-term trial. Int J Obes. 1989;13(2):165-71.

⁶⁹⁵ Piliang W G, Djojosoebagio S, Suprayogi A. Soybean hull and its effect on atherosclerosis in non-human primates (Macaca fascicularis). Biomed Environ Sci 1996 September;9(2-3):137-43.

⁶⁹⁶ Kim Y I. AGA technical review: impact of dietary fiber on colon cancer occurrence. Gastroenterology. 2000 June;118(6):1235-57.

⁶⁹⁷ Madar Z, Stark A. Dietary fiber and colorectal cancer. N Engl J. Med. Jun. 17, 1999;340(24):1925-6; discussion 1926.

⁶⁹⁸ Camire M E. Dietary fiber and colorectal cancer. N Engl J. Med. Jun. 17, 1999;340(24):1926.

⁶⁹⁹ Mohandas K M. Dietary fiber and colorectal cancer. N EngI J. Med. Jun. 17, 1999;340(24):1925; discussion 1926.

⁷⁰⁰ Heaton K W, Lewis S J. Dietary fiber and colorectal cancer. N Engl J. Med. Jun. 17, 1999;340(24):1925; discussion 1926.

⁷⁰¹ Cummings J H, Southgate D A. Dietary fiber and colorectal cancer. N Engl J. Med. Jun. 17, 1999;340(24):1925; discussion 1926.

⁷⁰² Ravin N D. Dietary fiber and colorectal cancer. N Engl J. Med. Jun. 17, 1999;340(24):1924-5; discussion 1926.

⁷⁰³ Reddy B S. Role of dietary fiber in colon cancer: an overview. Am J. Med. Jan. 25, 1999; 106(1 A): 16S-19S; discussion 50S-51S.

⁷⁰⁴ Reddy B S. Prevention of colon carcinogenesis by components of dietary fiber. Anticancer Res. 1999 September-October; 19(5A):3681-3.

⁷⁰⁵ Earnest D L, Einspahr J G, Alberts D S. Protective role of wheat bran fiber: data from marker trials. Am J. Med. Jan. 25, 1999;106(IA):32S-37S.

⁷⁰⁶ Kritchevsky D. Protective role of wheat bran fiber: preclinical data. Am J. Med. Jan. 25, 1999;106(1A):28S-31S.

⁷⁰⁷ Cohen L A. Dietary fiber and breast cancer. Anticancer Res. 1999 September-October; 19(5A):3685-8.

⁷⁰⁸ Wolin M J, Miller T L, Yerry S, Zhang Y, Bank S, Weaver G A. Changes of fermentation pathways of fecal microbial communities associated with a drug treatment that increases dietary starch in the human colon. Appl Environ Microbiol 1999 July;65(7):2807-12.

⁷⁰⁹ Wolever T M, Chiasson J L, Josse R G, Hunt J A, Palmason C, Rodger N W, Ross S A, Ryan E A, Tan M H. Small weight loss on long-term acarbose therapy with no change in dietary pattern or nutrient intake of individuals with non-insulin-dependent diabetes. Int J Obes Relat Metab Disord. 1997 September;21(9):756-63.

⁷¹⁰ Huyer G, Liu S, Kelly J, Moffat J, Payette P, Kennedy B, Tsaprailis G, Gresser M J, Ramachandran C. Mechanism of inhibition of protein-tyrosine phosphatases by vanadate and pervanadate. J Biol Chem Jan. 10, 1997;272(2):843-51.

⁷¹¹ Wang Y Z, Bonner J C. Mechanism of extracellular signal-regulated kinase (ERK)-1 and ERK-2 activation by vanadium pentoxide in rat pulmonary myofibroblasts. Am J Respir Cell Mol. Biol. 2000 May;22(5):590-6.

⁷¹² Zhao Z, Tan Z, Diltz C D, You M, Fischer E H. Activation of mitogen-activated protein (MAP) kinase pathway by pervanadate, a potent inhibitor of tyrosine phosphatases. J. Biol. Chem. Sep. 6, 1996;271(36):22251-5.

⁷¹³ Pandey S K, Chiasson J L, Srivastava A K. Vanadium salts stimulate mitogen-activated protein (MAP) kinases and ribosomal S6 kinases. Mol Cell Biochem. Dec. 6-20, 1995;153(1-2):69-78.

⁷¹⁴ D'Onofrio F, Le M Q, Chiasson J L, Srivastava A K. Activation of mitogen activated protein (MAP) kinases by vanadate is independent of insulin receptor autophosphorylation. FEBS Lett. Mar. 7, 1994;340(3):269-75.

⁷¹⁵ Darville M I, Antoine I V, Rousseau G G. Characterization of an enhancer upstream from the muscle-type promoter of a gene encoding 6-phosphofructo-2-kinase/fructose-2,6-bisphosphatase. Nucleic Acids Res. Jul. 25, 1992;20(14):3575-83.

⁷¹⁶ Dupriez V J, Darville M I, Antoine I V, Gegonne A, Ghysdael J, Rousseau G G. Characterization of a hepatoma mRNA transcribed from a third promoter of a 6-phosphofructo-2-kinase/fructose-2,6-bisphosphatase-encoding gene and controlled by ets oncogene-related products. Proc Natl Acad Sci U S A. Sep. 1, 1993;90(17):8224-8.

⁷¹⁷ Miralpeix M, Carballo E, Bartrons R, Crepin K, Hue L, Rousseau G G. Oral administration of vanadate to diabetic rats restores liver 6-phosphofructo-2-kinase content and mRNA. Diabetologia. 1992 March;35(3):243-8.

⁷¹⁸ Inoue H, Kaku K, Matsutani A, Tao T, Ayame H, Kaneko T. Insulin-like effects of vanadate on rat liver 6-phosphofructo-2-kinase/fructose-2,6-bisphosphatase mRNA and protein inductions in diabetic rats. Endocr J. 1994 February;41(1):75-82.

⁷¹⁹ Herrman C E, Sanders R A, Klaunig J E, Schwarz L R, Watkins J B 3rd. Decreased apoptosis as a mechanism for hepatomegaly in streptozotocin-induced diabetic rats. Toxicol Sci 1999 July;50(1):146-51.

⁷²⁰ Pugazhenthi S, Tanha F, Dahl B, Khandelwal R L. Decrease in protein tyrosine phosphatase activities in vanadate-treated obese Zucker (fa/fa) rat liver. Mol Cell Biochem. Dec. 6-20, 1995;153(1-2):125-9.

⁷²¹ McNeill J H, Orvig C. Bis(maltolato)oxovanadium compositions for the treatment of elevated blood sugar. U.S. Pat. No. 5,527,790, Jun. 18, 1996.

⁷²² Dai S, Thompson K H, McNeill J H. One-year treatment of streptozotocin-induced diabetic rats with vanadyl sulphate. Pharmacol Toxicol 1994 February;74(2):101-9.

⁷²³ Bhanot S, McNeill J H. Vanadyl sulfate lowers plasma insulin and blood pressure in spontaneously hypertensive rats. Hypertension. 1994 November;24(5):625-32.

⁷²⁴ Cruz T F, Morgan A, Min W. In vitro and in vivo antineoplastic effects of orthovanadate. Mol Cell Biochem. Dec. 6-20, 1995; 153(1-2): 161-6.

⁷²⁵ Bishayee A, Chatterjee M. Inhibitory effect of vanadium on rat liver carcinogenesis initiated with diethylnitrosamine and promoted by phenobarbital. BrJ Cancer. 1995 June;71(6):1214-20.

⁷²⁶ Liasko R, Kabanos T A, Karkabounas S, Malamas M, Tasiopoulos A J, Stefanou D, Collery P, Evangelou A. Beneficial effects of a vanadium complex with cysteine, administered at low doses on benzo(alpha)pyrene-induced leiomyosarcomas in Wistar rats. Anticancer Res. 1998 September-October;18(5A):3609-13.

⁷²⁷ Goldfine A B, Simonson D C, Folli F, Patti M E, Kahn C R. In vivo and in vitro studies of vanadate in human and rodent diabetes mellitus. Mol Cell Biochem. Dec. 6-20, 1995;153(1-2):217-31.

⁷²⁸ Brichard S M, Henquin J C. The role of vanadium in the management of diabetes. Trends Pharmacol Sci. 1995 August;16(8):265-70.

⁷²⁹ Elchebly M, Payette P, Michaliszyn E, Cromlish W, Collins S, Loy A L, Normandin D, Cheng A, Himms-Hagen J, Chan C C, Ramachandran C, Gresser M J, Tremblay M L, Kennedy B P. Increased insulin sensitivity and obesity resistance in mice lacking the protein tyrosine phosphatase-1 B gene. Science. Mar. 5, 1999;283(5407): 1544-8.

⁷³⁰ Klaman L D, Boss O, Peroni O D, Kim J K, Martino J L, Zabolotny J M, Moghal N, Lubkin M, Kim Y B, Sharpe A H, Stricker-Krongrad A, Shulman G I, Neel B G, Kahn B B. Increased energy expenditure, decreased adiposity, and tissue-specific insulin sensitivity in protein-tyrosine phosphatase IB-deficient mice. Mol Cell Biol. 2000 August;20(15):5479-89.

⁷³¹ Klaman L D, Boss O, Peroni O D, Kim J K, Martino J L, Zabolotny J M, Moghal N, Lubkin M, Kim Y B, Sharpe A H, Stricker-Krongrad A, Shulman G I, Neel B G, Kahn B B. Increased energy expenditure, decreased adiposity, and tissue-specific insulin sensitivity in protein-tyrosine phosphatase 1B-deficient mice. Mol Cell Biol. 2000 August;20(15):5479-89.

⁷³² Prasad K, Laxdal V A, Yu M, Raney B L. Evaluation of hydroxyl radical-scavenging property of garlic. Mol Cell Biochem. Jan. 12, 1996;154(1):55-63.

⁷³³ Ide N, Lau B H. Aged garlic extract attenuates intracellular oxidative stress. Phytomedicine 1999 May;6(2): 125-31.

⁷³⁴ Efendy J L, Simmons D L, Campbell G R, Campbell J H. The effect of the aged garlic extract, ‘Kyolic’, on the development of experimental atherosclerosis. Atherosclerosis. Jul. 11, 1997;132(1):37-42.

⁷³⁵ Jain R C, Konar DBEffect of garlic oil in experimental cholesterol atherosclerosis. Atherosclerosis. 1978 February;29(2):125-9.

⁷³⁶ Jain R C. Onion and garlic in experimental cholesterol induced atherosclerosis. Indian J Med Res. 1976 October;64(10):1509-15.

⁷³⁷ Bordia A, Arora S K, Kothari L K, Jain K C, Rathore B S, Rathore A S, Dube M K, Bhu N. The protective action of essential oils of onion and garlic in cholesterol-fed rabbits. Atherosclerosis. 1975 July-August;22(1): 103-9.

⁷³⁸ Breithaupt-Grogler K, Ling M, Boudoulas H, Belz G G. Protective effect of chronic garlic intake on elastic properties of aorta in the elderly. Circulation. Oct. 21, 1997;96(8):2649-55.

⁷³⁹ Farrar D J, Bond M G, Riley W A, Sawyer J K. Anatomic correlates of aortic pulse wave velocity and carotid artery elasticity during atherosclerosis progression and regression in monkeys. Circulation. 1991 May;83(5): 1754-63.

⁷⁴⁰ Ali M, Thomson M, Afzal M. Garlic and onions: their effect on eicosanoid metabolism and its clinical relevance. Prostaglandins Leukot Essent Fatty Acids. 2000 February;62(2):55-73.

⁷⁴¹ Fleischauer A T, Poole C, Arab L. Garlic consumption and cancer prevention: meta-analyses of colorectal and stomach cancers. Am J Clin Nutr. 2000 October;72(4):1047-52.

⁷⁴² Singh A, Shukla Y. Antitumour activity of diallyl sulfide on polycyclic aromatic hydrocarbon-induced mouse skin carcinogenesis. Cancer Lett. Sep. 25, 1998;131(2):209-14.

⁷⁴³ Singh S V, Mohan R R, Agarwal R, Benson P J, Hu X, Rudy M A, Xia H, Katoh A, Srivastava S K, Mukhtar H, Gupta V, Zaren H A. Novel anti-carcinogenic activity of an organosulfide from garlic: inhibition of H-RAS oncogene transformed tumor growth in vivo by diallyl disulfide is associated with inhibition of p₂₁^{H-ras} processing. Biochem Biophys Res Commun. Aug. 14, 1996;225(2):660-5.

⁷⁴⁴ Spector S A. Oral ganciclovir. Adv Exp Med Biol 1999;458:121-7.

⁷⁴⁵ Leflore S, Anderson P L, Fletcher C V. A risk-benefit evaluation of aciclovir for the treatment and prophylaxis of herpes simplex virus infections. Drug Saf 2000 August;23(2):131-42.

⁷⁴⁶ Kesson A M. Use of aciclovir in herpes simplex virus infections. J Paediatr Child Health. 1998 February;34(1):9-13.

⁷⁴⁷ Ormrod D, Scott L J, Perry C M. Valaciclovir: a review of its long term utility in the management of genital herpes simplex virus and cytomegalovirus infections. Drugs 2000 April;59(4):839-63.

⁷⁴⁸ Bell A R. Valaciclovir update. Adv Exp Med Biol. 1999;458:149-57.

⁷⁴⁹ Sacks S L, Wilson B. Famciclovir/penciclovir. Adv Exp Med Biol. 1999;458:135-47.

⁷⁵⁰ Reddehase M J, Balthesen M, Rapp M, Jonjic S, Pavic I, Koszinowski U H. The conditions of primary infection define the load of latent viral genome in organs and the risk of recurrent cytomegalovirus disease. J Exp Med Jan. 1, 1994;179(1):185-93.

⁷⁵¹ Collins T, Pomeroy C, Jordan M C. Detection of latent cytomegalovirus DNA in diverse organs of mice. J Infect Dis 1993 September;168(3):725-9.

⁷⁵² Steffens H P, Kurz S, Holtappels R, Reddehase M J. Preemptive CD8 T-cell immunotherapy of acute cytomegalovirus infection prevents lethal disease, limits the burden of latent viral genomes, and reduces the risk of virus recurrence. J. Virol. 1998 March;72(3):1797-804.

⁷⁵³ Thackray A M, Field H J. The effects of antiviral therapy on the distribution of herpes simplex virus type 1 to ganglionic neurons and its consequences during, immediately following and several months after treatment. J Gen Virol 2000 October;81 Pt 10:2385-96.

⁷⁵⁴ Thackray A M, Field H J. Further evidence from a murine infection model that famciclovir interferes with the establishment of HSV-1 latent infections. J Antimicrob Chemother. 2000 June;45(6):825-33.

⁷⁵⁵ Thackray A M, Field H J. Effects of famciclovir and valacyclovir on herpes simplex virus type 1 infection, latency, and reactivation in mice: how dissimilar are study results? J Infect Dis 2000 April;181(4):1517-8.

⁷⁵⁶ Field H J, Thackray A M. Early therapy with valaciclovir or famciclovir reduces but does not abrogate herpes simplex virus neuronal latency. Nucleosides Nucleotides Nucleic Acids. 2000 January-February;19(1-2):461-70.

⁷⁵⁷ Thackray A M, Field H J. Famciclovir and valaciclovir differ in the prevention of herpes simplex virus type I latency in mice: a quantitative study. Antimicrob Agents Chemother 1998 July;42(7): 1555-62.

⁷⁵⁸ LeBlanc R A, Pesnicak L, Godleski M, Straus S E. Treatment of HSV-1 infection with immunoglobulin or acyclovir: comparison of their effects on viral spread, latency, and reactivation. Virology. Sep. 15, 1999;262(1):230-6.

⁷⁵⁹ Bowden R A. Cytomegalovirus infections in transplant patients: methods of prevention of primary cytomegalovirus. Transplant Proc 1991 June;23(3 Suppl 3):136-8, discussion 138.

⁷⁶⁰ Chou S W, Norman D J. The influence of donor factors other than serologic status on transmission of cytomegalovirus to transplant recipients. Transplantation 1988 July;46(1):89-93.

⁷⁶¹ Chou S W. Cytomegalovirus infection and reinfection transmitted by heart transplantation. J Infect Dis. 1987 May; 155(5): 1054-6.

⁷⁶² Chou S W. Acquisition of donor strains of cytomegalovirus by renal-transplant recipients. N Engl J. Med. May 29, 1986;314(22):1418-23.

⁷⁶³ Grundy J E, Lui S F, Super M, Berry N J, Sweny P, Fernando O N, Moorhead J, Griffiths P D. Symptomatic cytomegalovirus infection in seropositive kidney recipients: reinfection with donor virus rather than reactivation of recipient virus. Lancet Jul. 16, 1988;2(8603):132-5.

⁷⁶⁴ Grundy J E, Super M, Lui S, Sweny P, Griffiths P D. The source of cytomegalovirus infection in seropositive renal allograft recipients is frequently the donor kidney. Transplant Proc. 1987 February; 19(1 Pt 3):2126-8.

⁷⁶⁵ Grundy J E, Super M, Lui S F, Griffiths P D. Donor strains of cytomegalovirus in renal-transplant recipients. N EngI J. Med. Nov. 6, 1986;315(19):1229.

⁷⁶⁶ Valantine H A, Gao S Z, Menon S G, Renlund D G, Hunt S A, Oyer P, Stinson E B, Brown B W Jr, Merigan T C, Schroeder J S. Impact of prophylactic immediate posttransplant ganciclovir on development of transplant atherosclerosis: a post hoc analysis of a randomized, placebo-controlled study. Circulation. Jul. 6, 1999;100(1):61-6.

⁷⁶⁷ Sia I G, Patel R. New strategies for prevention and therapy of cytomegalovirus infection and disease in solid-organ transplant recipients. Clin Microbiol Rev. 2000 January;13(1):83-121.

⁷⁶⁸ Perry C M, Noble S. Didanosine: an updated review of its use in HIV infection. Drugs. 1999 December;58(6): 1099-135.

⁷⁶⁹ Bruisten S M, Reiss P, Loeliger A E, van Swieten P, Schuurman R, Boucher C A, Weverling G J, Huisman J G. Cellular proviral HIV type 1 DNA load persists after long-term RT-inhibitor therapy in HIV type 1 infected persons. AIDS Res Hum Retroviruses. Aug. 10, 1998;14(12):1053-8.

⁷⁷⁰ Magnani M, Rossi L, Fraternale A, Casabianca A, Brandi G, Benatti U, De Flora A. Targeting antiviral nucleotide analogues to macrophages. J Leukoc Biol 1997 July;62(1):133-7.

⁷⁷¹ Pauza C D, Trivedi P, McKechnie T S, Richman D D, Graziano F M. 2-LTR circular viral DNA as a marker for human immunodeficiency virus type 1 infection in vivo. Virology. 1994 December;205(2):470-8.

⁷⁷² Chun T W, Stuyver L, Mizell S B, Ehler L A, Mican J A, Baseler M, Lloyd A L, Nowak M A, Fauci A S. Presence of an inducible HIV-1 latent reservoir during highly active antiretroviral therapy. Proc Natl Acad Sci U S A. Nov. 25, 1997;94(24):13193-7.

⁷⁷³ Guo N L, Lu D P, Woods G L, Reed E, Zhou G Z, Zhang L B, Waldman R H. Demonstration of the anti-viral activity of garlic extract against human cytomegalovirus in vitro. Chin Med J (Engl) 1993 February; 106(2):93-6.

⁷⁷⁴ Weber N D, Andersen D O, North J A, Murray B K, Lawson L D, Hughes B G. In vitro virucidal effects of Allium sativum (garlic) extract and compounds. Planta Med. 1992 October;58(5):417-23.

⁷⁷⁵ Marchant A, Goetghebuer T, Ota M O, Wolfe I, Ceesay S J, De Groote D, Corrah T, Bennett S, Wheeler J, Huygen K, Aaby P, McAdam K P, Newport M J. Newborns develop a Th 1-type immune response to Mycobacterium bovis bacillus Calmette-Guerin vaccination. J. Immunol. Aug. 15, 1999;163(4):2249-55.

⁷⁷⁶ Starr S E, Visintine A M, Tomeh M O, Nahmias A J. Effects of immunostimulants on resistance of newborn mice to herpes simplex type 2 infection. Proc Soc Exp Biol Med. 1976 May; 152(1):57-60.

⁷⁷⁷ Aaby P, Shaheen S O, Heyes C B, Goudiaby A, Hall A J, Shiell A W, Jensen H, Marchant A. Early BCG vaccination and reduction in atopy in Guinea-Bissau. Clin Exp Allergy 2000 May;30(5):644-50.

⁷⁷⁸ von Hertzen L C. Puzzling associations between childhood infections and the later occurrence of asthma and atopy. Ann Med 2000 September;32(6):397-400.

⁷⁷⁹ von Mutius E, Pearce N, Beasley R, Cheng S, von Ehrenstein O, Bjorksten B, Weiland S. International patterns of tuberculosis and the prevalence of symptoms of asthma, rhinitis, and eczema. Thorax 2000 June;55(6):449-53.

⁷⁸⁰ von Hertzen L, Klaukka T, Mattila H, Haahtela T. Mycobacterium tuberculosis infection and the subsequent development of asthma and allergic conditions. J Allergy Clin Immunol. 1999 December;104(6):1211-4.

⁷⁸¹ Scanga C B, Le Gros G. Development of an asthma vaccine: research into BCG. Drugs. 2000 June;59(6):1217-21.

⁷⁸² Shehadeh N, Etzioni A, Cahana A, Teninboum G, Gorodetsky B, Barzilai D, Karnieli E. Repeated BCG vaccination is more effective than a single dose in preventing diabetes in non-obese diabetic (NOD) mice. Isr J Med Sci. 1997 November;33(11):711-5.

⁷⁸³ Qin H Y, Singh B. BCG vaccination prevents insulin-dependent diabetes mellitus (IDDM) in NOD mice after disease acceleration with cyclophosphamide. J Autoimmun. 1997 June;10(3):271-8.

⁷⁸⁴ Harada M, Kishimoto Y, Makino S. Prevention of overt diabetes and insulitis in NOD mice by a single BCG vaccination. Diabetes Res Clin Pract. 1990 January;8(2):85-9.

⁷⁸⁵ Hiltunen M, Lonnrot M, Hyoty H. Immunisation and type I diabetes mellitus: is there a link? Drug Saf 1999 March;20(3):207-12.

⁷⁸⁶ Martins T C, Aguas A P. Mechanisms of Mycobacterium avium-induced resistance against insulin-dependent diabetes mellitus (IDDM) in non-obese diabetic (NOD) mice: role of Fas and Th1 cells. Clin Exp Immunol 1999 February;115(2):248-54.

⁷⁸⁷ Bras A, Aguas A P. Diabetes-prone NOD mice are resistant to Mycobacterium avium and the infection prevents autoimmune disease. Immunology. 1996 September;89(1):20-5.

⁸⁸⁸ Pabst H F, Spady D W, Pilarski L M, Carson M M, Beeler J A, Krezolek M P. Differential modulation of the immune response by breast- or formula-feeding of infants. Acta Paediatr. 1997 December;86(12):1291-7.

⁷⁸⁹ Pabst H F. Immunomodulation by breast-feeding. Pediatr Infect Dis J. 1997 October;16(10):991-5.

⁷⁹⁰ Hawkes J S, Neumann M A, Gibson R A. The effect of breast-feeding on lymphocyte subpopulations in healthy term infants at 6 months of age. Pediatr Res 1999 May;45(5 Pt 1):648-51.

⁷⁹¹ Pettitt D J, Forman M R, Hanson R L, Knowler W C, Bennett P H. Breastfeeding and incidence of non-insulin-dependent diabetes mellitus in Pima Indians. Lancet. Jul. 19, 1997;350(9072):166-8.

⁷⁹² von Kries R, Koletzko B, Sauerwald T, von Mutius E. Does breast-feeding protect against childhood obesity? Adv Exp Med Biol. 2000;478:29-39.

⁷⁹³ von Kries R, Koletzko B, Sauerwald T, von Mutius E, Barnert D, Grunert V, von Voss H. Breast-feeding and obesity: cross sectional study. BMJ. Jul. 17, 1999;319(7203):147-50.

We claim:

1. A method for evaluating the effectiveness of a compound for use in modulating the progression of a chronic disease, the method comprising the steps of:
a. selecting a cell;
b. introducing a polynucleotide foreign to said cell into said cell, or identifying a polynucleotide foreign to said cell in said cell;
c. incubating said cell in the presence and absence of said compound;
d. assaying microcompetition for GABP between said foreign polynucleotide and a polynucleotide natural to said cell;
e. selecting a compound that can modify said microcompetition.

2. The method of claim 1, wherein said compound is foreign to said cell.

3. The method of claim 1, wherein said compound is a synthetic compound.

4. The method of claim 1, wherein said introduction of a foreign polynucleotide into a cell is by performing an action selected from a group consisting of: changing the copy number of a polynucleotide in a cell, transfecting a cell with a foreign polynucleotide, infecting a cell with an active or inactive virus, chemically modifying a polynucleotide in the cell, mutating a polynucleotide in the cell, and modifying the binding affinity or avidity of a polynucleotide in the cell.

5. The method of claim 1, wherein said cell is an animal or human cell.

6. The method of claim 1, wherein said foreign polynucleotide is a viral polynucleotide.

7. The method of claim 1, wherein said foreign polynucleotide is the complete, or a fragment of the genome of a GABP virus.

8. The method of claim 1, wherein said foreign polynucleotide is selected from a group consisting of: a viral promoter, and a viral enhancer.

9. The method of claim 1, wherein said foreign polynucleotide is selected from a group consisting of: a promoter of a GABP virus, an enhancer of a GABP virus, and a viral polynucleotide that includes an N-box.

10. The method of claim 1, wherein said assaying microcompetition is assaying the formation of a complex that includes GABP and said foreign polynucleotide.

11. The method of claim 1, wherein said assaying microcompetition is assaying the formation of a complex that includes GABP and a viral polynucleotide.

12. The method of claim 1, wherein said assaying microcompetition is assaying the expression of a gene, or gene fragment, where said gene is under the control of said foreign polynucleotide.

13. The method of claim 1, wherein said assaying microcompetition is assaying the activity of a gene product of a gene, or gene fragment, where said gene is under the control of said foreign polynucleotide.

14. The method of claim 1, wherein said assaying microcompetition is assaying the formation of a complex that includes GABP and said polynucleotide natural to said cell.

15. The method of claim 1, wherein said assaying microcompetition is assaying the expression of a gene, or gene fragment, where said gene is under the control of said polynucleotide natural to said cell.

16. The method of claim 1, wherein said assaying microcompetition is assaying the activity of a gene product of a gene, or gene fragment, where said gene is under the control of said polynucleotide natural to said cell.

17. The method of claim 1, wherein said assaying microcompetition for GABP is assaying the expression of a GABP regulated gene, or fragment of a GABP regulated gene.

18. The method of claim 1, wherein said assaying microcompetition for GABP is assaying the activity of a gene product of a GABP regulated gene, or fragment of a GABP regulated gene.

19. The method of claim 1, wherein said assaying microcompetition is assaying the copy number of said foreign polynucleotide.

20. The method of claim 1, wherein said assaying microcompetition is assaying the copy number of a GABP virus.

21. The method of claim 1, wherein said evaluating the effectiveness of a compound for use in modulating the progression of a chronic disease is evaluating the effectiveness of a compound for use in stimulating or inhibiting the progression of a chronic disease.

22. The method of claim 1, wherein said selecting a compound that can modify microcompetition is selecting a compound that can decrease or increase microcompetition for GABP.

23. The method of claim 1, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.

24. A method for evaluating the effectiveness of a compound for use in modulating the progression of a chronic disease, the method comprising the steps of:
a. selecting a polynucleotide where said polynucleotide is natural to a certain first organism; and where said polynucleotide is empty in respect to said first organism; and where said polynucleotide is foreign to a second organism;
b. introducing a polynucleotide foreign to said cell into said cell, or identifying a polynucleotide foreign to said cell in said cell;
c. incubating said cell in the presence and absence of said compound;
d. assaying microcompetition for GABP between said foreign polynucleotide and a polynucleotide natural to said cell;
e. selecting a compound that can modify said microcompetition.

25. The method of claim 24, wherein said compound is foreign to both said organisms.

26. The method of claim 24, wherein said compound is a synthetic compound.

27. The method of claim 24, wherein said introducing a foreign polynucleotide into a cell is by performing an action selected from a group consisting of: changing the copy number of a polynucleotide in a cell, transfecting a cell with a foreign polynucleotide, infecting a cell with an active or inactive virus, chemically modifying a polynucleotide in the cell, mutating a polynucleotide in the cell, and modifying binding affinity or avidity of a polynucleotide in the cell.

28. The method of claim 24, wherein said cell is an animal or human cell.

29. The method of claim 24, wherein said foreign polynucleotide is a viral polynucleotide.

30. The method of claim 24, wherein said foreign polynucleotide is the complete, or a fragment of the genome of a GABP virus.

31. The method of claim 24, wherein said foreign polynucleotide is selected from a group consisting of: a viral promoter, and a viral enhancer.

32. The method of claim 24, wherein said foreign polynucleotide is selected from a group consisting of: a promoter of a GABP virus, an enhancer of a GABP virus, and a viral polynucleotide that includes an N-box.

33. The method of claim 24, wherein said assaying microcompetition is assaying the formation of a complex that includes GABP and said foreign polynucleotide.

34. The method of claim 24, wherein said assaying microcompetition is assaying the formation of a complex that includes GABP and a viral polynucleotide.

35. The method of claim 24, wherein said assaying microcompetition is assaying the expression of a gene, or gene fragment, where said gene is under the control of said foreign polynucleotide.

36. The method of claim 24, wherein said assaying microcompetition is assaying the activity of a gene product of a gene, or gene fragment, where said gene is under the control of said foreign polynucleotide.

37. The method of claim 24, wherein said assaying microcompetition is assaying the formation of a complex that includes GABP and said polynucleotide natural to said cell.

38. The method of claim 24, wherein said assaying microcompetition is assaying the expression of a gene, or gene fragment, where said gene is under the control of said polynucleotide natural to said cell.

39. The method of claim 24, wherein said assaying microcompetition is assaying the activity of a gene product of a gene, or gene fragment, where said gene is under the control of said polynucleotide natural to said cell.

40. The method of claim 24, wherein said assaying microcompetition for GABP is assaying the expression of a GABP regulated gene, or fragment of a GABP regulated gene.

41. The method of claim 24, wherein said assaying microcompetition for GABP is assaying the activity of a gene product of a GABP regulated gene, or fragment of a GABP regulated gene.

42. The method of claim 24, wherein said assaying microcompetition is assaying the copy number of said foreign polynucleotide.

43. The method of claim 24, wherein said assaying microcompetition is assaying the copy number of a GABP virus.

44. The method of claim 24, wherein said evaluating the effectiveness of a compound for use in modulating the progression of a chronic disease is evaluating the effectiveness of a compound for use in stimulating or inhibiting the progression of a chronic disease.

45. The method of claim 24, wherein said selecting a compound that can modify microcompetition is selecting a compound that can decrease or increase microcompetition for GABP.

46. The method of claim 24, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.

47. A method for evaluating the effectiveness of a compound for use in modulating the progression of a chronic disease, the method comprising of:
1) performing an assay capable of identifying compounds that perform a function selected from the group consisting of:
a) modifying concentration of GABP;
b) modifying phosphorylation of GABP;
c) modifying affinity or avidity between members of the GABP family of proteins;
d) modifying affinity or avidity between GABP and p300/cbp;
e) modifying concentration of p300/cbp.
2) running sample compounds through said assay and selecting a compound that performs one of said functions.

48. The method of claim 47, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.

49. A method for evaluating the effectiveness of a compound for use in modulating the progression of a chronic disease, the method comprising of:
1) performing an assay capable of identifying compounds that perform a function selected from the group consisting of:
a) modifies expression of a GABP regulated gene;
b) modifies activity of a gene product of a GABP regulated gene.
2) running sample compounds through said assay and selecting a compound that performs one of said functions.

50. The method of claim 49, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.

51. A method for evaluating the effectiveness of a compound for use in modulating the progression of a chronic disease, the method comprising of:
1) performing an assay capable of identifying compounds that perform a function selected from the group consisting of:
a) modifying concentration of a GABP kinase;
b) modifying phosphorylation of a GABP kinase;
c) modifying concentration of a GABP phosphatase;
d) modifying phosphorylation of a GABP phosphatase;
e) modifying affinity or avidity between GABP and a GABP kinase;
f) modifying affinity or avidity between GABP and a GABP phosphatase;
g) modifying oxidative effects on GABP.
2) running sample compounds through said assay and selecting a compound that performs one of said functions.

52. The method of claim 51, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.

53. A method for monitoring the efficacy of a compound, or other treatments, in clinical trials for the treatment of a chronic disease in an animal or human subject, the method comprising assaying the effect of said compound on microcompetition for GABP between a polynucleotide natural to said subject and a polynucleotide foreign to said subject.

54. The method of claim 53, wherein said assay is carried out in a chemical mix or a cell.

55. The method of claim 53, wherein said chronic disease is selected from the group consisting of obesity, cancer, atherosclerosis, stroke, osteoarthritis, type II diabetes, type I diabetes, asthma, lupus, multiple sclerosis, and other autoimmune diseases.
